(19) United States
(12) Patent Application Publication
(10) Pub. No.: US 2023/0193390 A1
(43) Pub. Date: Jun. 22, 2023

(54) DETECTING A CHROMOSOME CONFIRMATION AS MARKER FOR FIBROSIS, E.G. SLERODERMA

(71) Applicant: OXFORD BIODYNAMICS PLC, Oxford Oxfordshire (GB)

(72) Inventors: Aroul Selvam Ramadass, Oxford (GB); Ewan Hunter, Oxford (GB); Alexandre Akoulitchev, Oxford (GB)

(73) Assignee: OXFORD BIODYNAMICS PLC, Oxford Oxfordshire (GB)

(21) Appl. No.: 18/000,243

(22) PCT Filed: Jun. 2, 2021
(86) PCT No.: PCT/GB2021/051356

§ 371 Date: Nov. 29, 2022

(30) Foreign Application Priority Data
Jun. 2, 2020 (GB) 2008269.9

(51) Int. Cl. C12Q 1/6883

(52) U.S. Cl. CPC C12Q 1/6883; C12Q 2600/112; C12Q 2600/136

(57) ABSTRACT

A process for analysing chromosome interactions relating to fibrosis.

### CROSS-REFERENCE

This application is a 371 National Stage filing and claims the benefit under 35 U.S.C. § 120 of International Application No. PCT/GB2021/051356, filed 2 Jun. 2021, which claims priority to Great Britain Application No. GB2008269.9, filed 2 Jun. 2020, each of which is incorporated herein by reference in its entirety.

### SEQUENCE LISTING INCORPORATION BY REFERENCE

The application herein incorporates by reference in its entirety the sequence listing material in the ASCII text file named “21.07.02 P104951WO01 Sequence Listing”, created Nov. 9, 2022, and having the size of 504 kilobytes, filed with this application.

### FIELD OF THE INVENTION

The invention relates to disease processes.

### BACKGROUND OF THE INVENTION

Fibrosis, also known as fibrotic scarring, is a pathological wound healing in which connective tissue replaces normal parenchymal tissue to the extent that it goes unchecked, leading to considerable tissue remodelling and the formation of permanent scar tissue. Sclerosis is a type of fibrosis in which there is the stiffening of a tissue or anatomical feature, usually caused by a replacement of the normal organ-specific tissue with connective tissue. The structure may be said to have undergone sclerotic changes or display sclerotic lesions, which refers to the process of sclerosis. Fibrosis is a complex condition where the regulatory and causative aspects cannot be easily elucidated.

The outcome of scleroderma depends on the extent of disease. Those with localised disease generally have a normal life expectancy. In those with systemic disease typical life expectancy is about 11 years from onset. Death is often due to lung, gastrointestinal, or heart complications.

### SUMMARY OF THE INVENTION

The inventors have identified chromosome conformation signatures relevant to the presence of fibrosis or the stage of fibrosis. Accordingly the invention provides a process for detecting a chromosome state which represents a subgroup in a population comprising determining whether a chromosome interaction relating to that chromosome state is present or absent within a defined region of the genome; and
wherein the subgroup relates to the stage of fibrosis and the chromosome interaction either:

(i) corresponds to any one of the chromosome interactions represented by any probe shown in Table 1, 3 or 4; and/or

(ii) is present in a 4,000 base region which comprises or which flanks (i);

and/or
wherein the subgroup relates to the presence of fibrosis and the chromosome interaction either:

(a) corresponds to any one of the chromosome interactions represented by any probe shown in Table 2, 5 or 6; and/or

(b) is present in a 4,000 base region which comprises or which flanks (a).

### BRIEF DESCRIPTION OF THE DRAWINGS

FIG. 1 shows the genetic locations and pathways for the early phenotype chromosome conformations. The top 35 pathways are shown for the genetic locations associated to significant chromosome conformations for the early phenotype.

FIG. 2 shows the genetic locations and pathways for the late phenotype chromosome conformations. The top 35 pathways are shown for the genetic locations associated to significant chromosome conformations for the late phenotype.

FIG. 3 shows an example of how the chromosome interaction typing may be carried out. The figure also shows how a probe (for example as described in the tables) corresponds to the ligated product generated by the method. The method steps are those of the 3C method.

### BRIEF DESCRIPTION OF THE TABLES

Table 1 shows 100 markers relating to early or late fibrosis, with 50 markers corresponding to each.

Table 2 shows 100 markers relating to presence of fibrosis, with 50 associated with disease and 50 associated with absence of disease.

Table 3 shows 100 markers associated with early fibrosis.

Table 4 shows 100 markers associated with late fibrosis.

Table 5 shows 100 markers associated with presence of fibrosis.

Table 6 shows 100 markers associated with the absence of fibrosis.

Table 7 shows SHAPLEY results for markers associated with early fibrosis.

Table 8 shows SHAPLEY results for markers associated with late fibrosis.

Table 9 shows SHAPLEY results for markers associated with presence of fibrosis.

Table 10 shows SHAPLEY results for markers associated with absence of fibrosis.

### DETAILED DESCRIPTION OF THE INVENTION

### Terms Used Herein

The chromosome interactions which are typed may be referred to as ‘markers’, ‘CCS’, ‘chromosome conformation signature’, ‘epigenetic interaction’ or ‘EpiSwitch markers’ herein. Such interactions are recognised in the art as regions of the chromosome coming together in a stable manner and this represents a distinct mode of regulation. A chromosome interaction can also be referred to as a ‘juxtaposition’ of chromosomes, chromosome ‘folding’ or ‘chromatin interaction’. Such interactions can be detected, for example, using the 3C (chromosome conformation capture) method.

The word ‘type’ will be interpreted as per the context, but will usually refer to detection of whether a specific chromosome interaction is present or absent.

Different subgroups are mentioned herein, but essentially the invention allows identification of groups relating to fibrosis, and in particular to early or late fibrosis, or to presence or absence of fibrosis. The invention therefore provides a method of diagnosis (or detecting) the stage of fibrosis or the presence or fibrosis.

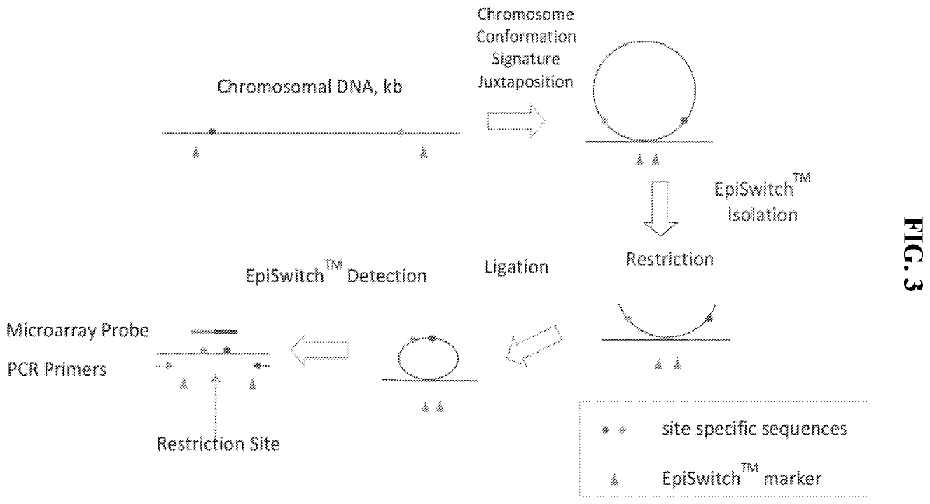

The chromosome interactions which are typed in the method of the invention are defined in Tables 1 to 6. They are defined by means of the probe sequences which detect the ligated product made by an EpiSwitch method (see FIG. 3). They are also defined by the position numbers of the interaction which are included within the probe name and they are also defined by the primer sequences which allow detection of the ligated sequence. The chromosome interaction can be defined by the ‘probe location’ given in the tables with reference to the chromosome number and the ‘Start’ and ‘End’ positions given for the chromosome regions which come together to form the interaction.

The word ‘method’ is used herein to refer to the ‘process’ of the invention, unless the context requires otherwise.

### Aspects of the Invention

The invention relates to determining different aspects of fibrosis, particularly in respect to the presence or stage of fibrosis. This determining is by typing any of the relevant markers disclosed herein, for example in Table 1 or 2, or preferred combinations of markers, or markers in defined specific regions disclosed herein. The typing may be of any of the markers shown in Table 3, 4, 5 or 6 or selections (combinations) of markers from these tables.

Specific number of markers may be chosen from any group of markers which is specifically disclosed herein. Preferred numbers of markers are at least 3, 5, 8, 10, 15 and at least 20. Preferred groups of markers are those shown in each table, or each part of a table (for example Table 1.a1), or all the markers associated with a distinct characteristic of fibrosis.

The invention includes a process of typing a patient to identify whether they have fibrosis and/or the stage of fibrosis. The invention includes diagnosis of an individual for any condition or stage of disease as defined herein (i.e. prognosis), which can be thought of as determining the subgroup they belong to. The fibrosis may be a skin fibrotic condition, which may preferably be scleroderma.

The invention also concerns a panel of epigenetic markers which relates to fibrosis. The panel may have been optimised in some way, for example by GLMNET analysis.

The invention therefore allows personalised therapy to be given to the patient which accurately reflects the patient's needs.

Any therapy, for example drug, which is mentioned herein may be administered to an individual based on the result of the process.

Marker sets are disclosed in the Tables and Figures. In one embodiment at least 10 markers from any disclosed marker set are used in the invention. In another embodiment at least 20% of the markers from any disclosed marker set are used in the invention.

### The Process of the Invention

The process of the invention comprises a typing system for detecting chromosome interactions relevant to fibrosis. This typing may be performed using the EpiSwitch™ system mentioned herein which is based on cross-linking regions of chromosome which have come together in the chromosome interaction, subjecting the chromosomal DNA to cleavage and then ligating the nucleic acids present in the cross-linked entity to derive a ligated nucleic acid with sequence from both the regions which formed the chromosomal interaction. Detection of this ligated nucleic acid allows determination of the presence or absence of a particular chromosome interaction. The ligated nucleic acid therefore acts as a marker for the presence of the chromosome interaction. Preferably the ligated nucleic acid is detected by PCR or a probe based method, including a qPCR method.

Any suitable typing method can be used for detecting the presence or absence of chromosome interactions, for example a method in which the proximity of the chromosomes in the interaction is detected and/or in which a marker that reflects chromosome interaction status is detected.

The chromosomal interactions may be identified using the process described herein in which populations of first and second nucleic acids are used. These nucleic acids can also be generated using EpiSwitch™ technology.

### The Epigenetic Interactions Relevant to the Invention

As used herein, the term ‘epigenetic’ and ‘chromosome’ interactions typically refer to interactions between distal regions of a chromosome, said interactions being dynamic and altering, forming or breaking depending upon the state of the region of the chromosome. That state will typically reflect the presence or stage of fibrosis.

In particular processes of the invention chromosome interactions are typically detected by first generating a ligated nucleic acid that comprises sequence from both regions of the chromosomes that are part of the interactions. In such processes the regions can be cross-linked by any suitable means. In a preferred aspect, the interactions are cross-linked using formaldehyde, but may also be cross-linked by any aldehyde, or D-Biotinoyl-e-aminocaproic acid-N-hydroxysuccinimide ester or Digoxigenin-3-O-methylcarbonyl-e-aminocaproic acid-N-hydroxysuccinimide ester. Para-formaldehyde can cross link DNA chains which are 4 Angstroms apart. Preferably the chromosome interactions are on the same chromosome. Typically the chromosome interactions are 2 to 10 Angstroms apart.

The chromosome interaction may reflect the status of the region of the chromosome, for example, if it is being transcribed or repressed in response to change of the physiological conditions. Chromosome interactions which are specific to subgroups as defined herein have been found to be stable, thus providing a reliable means of measuring the differences between the two subgroups.

In addition, chromosome interactions specific to a fibrosis characteristic will normally occur early in a biological process, for example compared to other epigenetic markers such as methylation or changes to binding of histone proteins. Thus the process of the invention is able to detect early stages of a biological process. This allows early intervention (for example treatment) which may as a consequence be more effective. Chromosome interactions also reflect the current state of the individual and therefore can be used to assess changes to disease status. Furthermore there is little variation in the relevant chromosome interactions between individuals within the same subgroup. Detecting chromosome interactions is highly informative with up to 50 different possible interactions per gene, and so processes of the invention can for example interrogate 500,000 different interactions.

### Preferred Marker Sets

Herein the term ‘marker’ or ‘biomarker’ refers to a specific chromosome interaction which can be detected (typed) in the invention. Specific markers are disclosed herein, any of which may be used in the invention. Further sets of markers may be used, for example in the combinations or numbers disclosed herein. The specific markers disclosed in the tables herein are preferred as well as markers presents in genes and regions mentioned in the tables herein are preferred. These may be typed by any suitable process, for example the PCR or probe based methods disclosed herein, including a qPCR method. The markers are defined herein by location or by probe and/or primer sequences.

### Location and Causes of Epigenetic Interactions

Epigenetic chromosomal interactions may overlap and include the regions of chromosomes shown to encode relevant or undescribed genes, but equally may be in intergenic regions. It should further be noted that the inventors have discovered that epigenetic interactions in all regions are equally important in determining the status of the chromosomal locus. These interactions are not necessarily in the coding region of a particular gene located at the locus and may be in intergenic regions.

The chromosome interactions which are detected in the invention could be caused by changes to the underlying DNA sequence, by environmental factors, DNA methylation, non-coding antisense RNA transcripts, non-mutagenic carcinogens, histone modifications, chromatin remodelling and specific local DNA interactions. The changes which lead to the chromosome interactions may be caused by changes to the underlying nucleic acid sequence, which themselves do not directly affect a gene product or the mode of gene expression. Such changes may be for example, SNPs within and/or outside of the genes, gene fusions and/or deletions of intergenic DNA, microRNA, and non-coding RNA. For example, it is known that roughly 20% of SNPs are in non-coding regions, and therefore the process as described is also informative in non-coding situation. In one aspect the regions of the chromosome which come together to form the interaction are less than 5 kb, 3 kb, 1 kb, 500 base pairs or 200 base pairs apart on the same chromosome.

The chromosome interaction which is detected is preferably within any of the genes in the regions defined by Table 1 or 2. The chromosome interaction which is detected is preferably within any of the genes in the regions defined by Table 3, 4, 5 or 6. However it may also be upstream or downstream of the gene, for example up to 50,000, up to 30,000, up to 20,000, up to 10,000 or up to 5000 bases upstream or downstream from the gene or from the coding sequence.

### Subgroups, Time Points and Personalised Treatment

Typing according to the process of the invention may be carried out at multiple time points, for example to monitor the progression of the disease. This may be at one or more defined time points, for example at at least 1, 2, 5, 8 or 10 different time points. The durations between at least 1, 2, 5 or 8 of the time points may be at least 5, 10, 20, 50, 80 or 100 days. Typically there are 3 time points at least 50 days apart.

As used herein, a “subgroup” preferably refers to a population subgroup (a subgroup in a population), more preferably a subgroup in the population of a particular animal such as a particular eukaryote, or mammal. Most preferably, a “subgroup” refers to a subgroup in the human population. Therefore the most preferred type of individuals for all aspects of the inventions are humans.

The invention includes detecting and treating particular subgroups in a population. The inventors have discovered that chromosome interactions differ between subsets (for example at least two subsets) in the relevant population. Identifying these differences will allow physicians to categorize their patients as a part of one subset of the population. The invention therefore provides physicians with a process of personalizing medicine for the patient based on their epigenetic chromosome interactions.

Such testing may be used to select how to subsequently treat the patient, for example the type of drug and/or its dose and/or its frequency of administration.

### Generating Ligated Nucleic Acids

Certain aspects of the invention utilise ligated nucleic acids, in particular ligated DNA. These comprise sequences from both of the regions that come together in a chromosome interaction and therefore provide information about the interaction. The EpiSwitch™ process described herein uses generation of such ligated nucleic acids to detect chromosome interactions.

Thus a process of the invention may comprise a step of generating ligated nucleic acids (e.g. DNA) by the following steps (including a process comprising these steps):

(i) cross-linking of epigenetic chromosomal interactions present at the chromosomal locus, preferably in vitro;

(ii) optionally isolating the cross-linked DNA from said chromosomal locus;

(iii) subjecting said cross-linked DNA to cutting, for example by restriction digestion with an enzyme that cuts it at least once (in particular an enzyme that cuts at least once within said chromosomal locus);

(iv) ligating said cross-linked cleaved DNA ends (in particular to form DNA loops); and

(v) optionally identifying the presence of said ligated DNA and/or said DNA loops, in particular using techniques such as PCR (polymerase chain reaction), to identify the presence of a specific chromosomal interaction.

These steps may be carried out to detect the chromosome interactions for any aspect mentioned herein. The steps may also be carried out to generate the first and/or second set of nucleic acids mentioned herein.

PCR (polymerase chain reaction) may be used to detect or identify the ligated nucleic acid, for example the size of the PCR product produced may be indicative of the specific chromosome interaction which is present, and may therefore be used to identify the status of the locus. In preferred aspects the primers shown in Table 1 or 2 are used. In other preferred aspects the primers shown in Table 3, 4, 5 or 6 are used.

The skilled person will be aware of numerous restriction enzymes which can be used to cut the DNA within the chromosomal locus of interest. It will be apparent that the particular enzyme used will depend upon the locus studied and the sequence of the DNA located therein. A non-limiting example of a restriction enzyme which can be used to cut the DNA as described in the present invention is Taql.

### EpiSwitch™ Technology

The EpiSwitch™ Technology also relates to the use of microarray EpiSwitch™ marker data in the detection of epigenetic chromosome conformation signatures specific for phenotypes. Aspects such as EpiSwitch™ which utilise ligated nucleic acids in the manner described herein have several advantages. They have a low level of stochastic noise, for example because the nucleic acid sequences from the first set of nucleic acids of the present invention either hybridise or fail to hybridise with the second set of nucleic acids. This provides a binary result permitting a relatively simple way to measure a complex mechanism at the epigenetic level. EpiSwitch™ technology also has fast processing time and low cost. In one aspect the processing time is 3 hours to 6 hours.

### Samples and Sample Treatment

The process of the invention will normally be carried out on a sample. The sample may be obtained at a defined time point, for example at any time point defined herein. The sample will normally contain DNA from the individual. It will normally contain cells. In one aspect a sample is obtained by minimally invasive means, and may for example be a blood sample. DNA may be extracted and cut up with a standard restriction enzyme. This can pre-determine which chromosome conformations are retained and will be detected with the EpiSwitch™ platforms. Due to the synchronisation of chromosome interactions between tissues and blood, including horizontal transfer, a blood sample can be used to detect the chromosome interactions in tissues, such as tissues relevant to disease.

### Properties of Nucleic Acids of the Invention

The invention relates to certain nucleic acids, such as the ligated nucleic acids which are described herein as being used or generated in the process of the invention. These may be the same as, or have any of the properties of, the first and second nucleic acids mentioned herein with reference to a screening method described below. The nucleic acids of the invention typically comprise two portions each comprising sequence from one of the two regions of the chromosome which come together in the chromosome interaction. Typically each portion is at least 8, 10, 15, 20, 30 or 40 nucleotides in length, for example 10 to 40 nucleotides in length. Preferred nucleic acids comprise sequence from any of the genes mentioned in any of the tables. Typically preferred nucleic acids comprise the specific probe sequences mentioned in Table 1, 2, 3, 4, 5 or 6; or fragments and/or homologues of such sequences.

Preferably the nucleic acids are DNA. It is understood that where a specific sequence is provided the invention may use the complementary sequence as required in the particular aspect. Preferably the nucleic acids are DNA. It is understood that where a specific sequence is provided the invention may use the complementary sequence as required in the particular aspect.

The primers shown in Table 1 and 2 may also be used in the invention as mentioned herein. In one aspect primers are used which comprise any of: the sequences shown in Table 1 or 2; or fragments and/or homologues of any sequence shown in Table 1 or 2. The primers shown in Table 3, 4, 5 or 6 may also be used in the invention as mentioned herein. In one aspect primers are used which comprise any of: the sequences shown in Table 3, 4, 5 or 6; or fragments and/or homologues of any sequence shown in Table 3, 4, 5 or 6.

### The Second Set of Nucleic Acids—the ‘Index’ Sequences

The second set of nucleic acid sequences has the function of being a set of index sequences, and is essentially a set of nucleic acid sequences which are suitable for identifying subgroup specific sequence. They can represents the ‘background’ chromosomal interactions and might be selected in some way or be unselected. They are in general a subset of all possible chromosomal interactions.

The second set of nucleic acids may be derived by any suitable process. They can be derived computationally or they may be based on chromosome interaction in individuals. They typically represent a larger population group than the first set of nucleic acids. In one particular aspect, the second set of nucleic acids represents all possible epigenetic chromosomal interactions in a specific set of genes. In another particular aspect, the second set of nucleic acids represents a large proportion of all possible epigenetic chromosomal interactions present in a population described herein. In one particular aspect, the second set of nucleic acids represents at least 50% or at least 80% of epigenetic chromosomal interactions in at least 20, 50, 100 or 500 genes, for example in 20 to 100 or 50 to 500 genes.

The second set of nucleic acids typically represents at least 100 possible epigenetic chromosome interactions which modify, regulate or in any way mediate a phenotype in population. The second set of nucleic acids may represent chromosome interactions that affect a disease state (typically relevant to diagnosis or prognosis) in a species. The second set of nucleic acids typically comprises sequences representing epigenetic interactions both relevant and not relevant to a prognosis subgroup.

In one particular aspect the second set of nucleic acids derive at least partially from naturally occurring sequences in a population, and are typically obtained by in silico processes. Said nucleic acids may further comprise single or multiple mutations in comparison to a corresponding portion of nucleic acids present in the naturally occurring nucleic acids. Mutations include deletions, substitutions and/or additions of one or more nucleotide base pairs. In one particular aspect, the second set of nucleic acids may comprise sequence representing a homologue and/or orthologue with at least 70% sequence identity to the corresponding portion of nucleic acids present in the naturally occurring species. In another particular aspect, at least 80% sequence identity or at least 90% sequence identity to the corresponding portion of nucleic acids present in the naturally occurring species is provided.

### Properties of the Second Set of Nucleic Acids

In one particular aspect, there are at least 100 different nucleic acid sequences in the second set of nucleic acids, preferably at least 1000, 2000 or 5000 different nucleic acids sequences, with up to 100,000, 1,000,000 or 10,000,000 different nucleic acid sequences. A typical number would be 100 to 1,000,000, such as 1,000 to 100,000 different nucleic acids sequences. All or at least 90% or at least 50% or these would correspond to different chromosomal interactions.

In one particular aspect, the second set of nucleic acids represent chromosome interactions in at least 20 different loci or genes, preferably at least 40 different loci or genes, and more preferably at least 100, at least 500, at least 1000 or at least 5000 different loci or genes, such as 100 to 10,000 different loci or genes. The lengths of the second set of nucleic acids are suitable for them to specifically hybridise according to Watson Crick base pairing to the first set of nucleic acids to allow identification of chromosome interactions specific to subgroups. Typically the second set of nucleic acids will comprise two portions corresponding in sequence to the two chromosome regions which come together in the chromosome interaction. The second set of nucleic acids typically comprise nucleic acid sequences which are at least 10, preferably 20, and preferably still 30 bases (nucleotides) in length. In another aspect, the nucleic acid sequences may be at the most 500, preferably at most 100, and preferably still at most 50 base pairs in length. In a preferred aspect, the second set of nucleic acids comprises nucleic acid sequences of between 17 and 25 base pairs. In one aspect at least 100, 80% or 50% of the second set of nucleic acid sequences have lengths as described above. Preferably the different nucleic acids do not have any overlapping sequences, for example at least 100%, 90%, 80% or 50% of the nucleic acids do not have the same sequence over at least 5 contiguous nucleotides.

Given that the second set of nucleic acids acts as an ‘index’ then the same set of second nucleic acids may be used with different sets of first nucleic acids which represent subgroups for different characteristics, i.e. the second set of nucleic acids may represent a ‘universal’ collection of nucleic acids which can be used to identify chromosome interactions relevant to different characteristics.

### The First Set of Nucleic Acids

The first set of nucleic acids are typically from subgroups relevant to fibrosis. The first nucleic acids may have any of the characteristics and properties of the second set of nucleic acids mentioned herein. The first set of nucleic acids is normally derived from samples from the individuals which have undergone treatment and processing as described herein, particularly the EpiSwitch™ cross-linking and cleaving steps. Typically the first set of nucleic acids represents all or at least 80% or 50% of the chromosome interactions present in the samples taken from the individuals.

Typically, the first set of nucleic acids represents a smaller population of chromosome interactions across the loci or genes represented by the second set of nucleic acids in comparison to the chromosome interactions represented by second set of nucleic acids, i.e. the second set of nucleic acids is representing a background or index set of interactions in a defined set of loci or genes.

### Library of Nucleic Acids

Any of the types of nucleic acid populations mentioned herein may be present in the form of a library comprising at least 200, at least 500, at least 1000, at least 5000 or at least 10000 different nucleic acids of that type, such as ‘first’ or ‘second’ nucleic acids. Such a library may be in the form of being bound to an array. The library may comprise some or all of the probes or primer pairs shown in Table 1 or 2. The library may comprise some or all of the probes or primer pairs shown in Table 3, 4, 5 or 6. The library may comprise all of the probe sequence from any of the tables disclosed herein.

### Hybridisation

The invention typically requires a means for allowing wholly or partially complementary nucleic acid sequences from the first set of nucleic acids and the second set of nucleic acids to hybridise. In one aspect all of the first set of nucleic acids is contacted with all of the second set of nucleic acids in a single assay, i.e. in a single hybridisation step. However any suitable assay can be used.

### Labelled Nucleic Acids and Pattern of Hybridisation

The nucleic acids mentioned herein may be labelled, preferably using an independent label such as a fluorophore (fluorescent molecule) or radioactive label which assists detection of successful hybridisation. Certain labels can be detected under UV light. The pattern of hybridisation, for example on an array described herein, represents differences in epigenetic chromosome interactions between the two subgroups, and thus provides a process of comparing epigenetic chromosome interactions and determination of which epigenetic chromosome interactions are specific to a subgroup in the population of the present invention.

The term ‘pattern of hybridisation’ broadly covers the presence and absence of hybridisation between the first and second set of nucleic acids, i.e. which specific nucleic acids from the first set hybridise to which specific nucleic acids from the second set, and so it not limited to any particular assay or technique, or the need to have a surface or array on which a ‘pattern’ can be detected.

The Chromosome Interactions which are Typed

The chromosome interactions which are typed are:

(i) those which specifically defined in any of Tables 1, 2, 3, 4, 5 or 6, for example either by probe sequence or by position numbers on the chromosome, and/or

(ii) those which are present in the genes or regions defined in any of Tables 1, 2, 3, 4 5 or 6, and/or

(iii) those present in a 4,000 base region which comprises or which flanks any specific chromosome interaction defined in any of Tables 1, 2, 3, 4, 5 or 6.

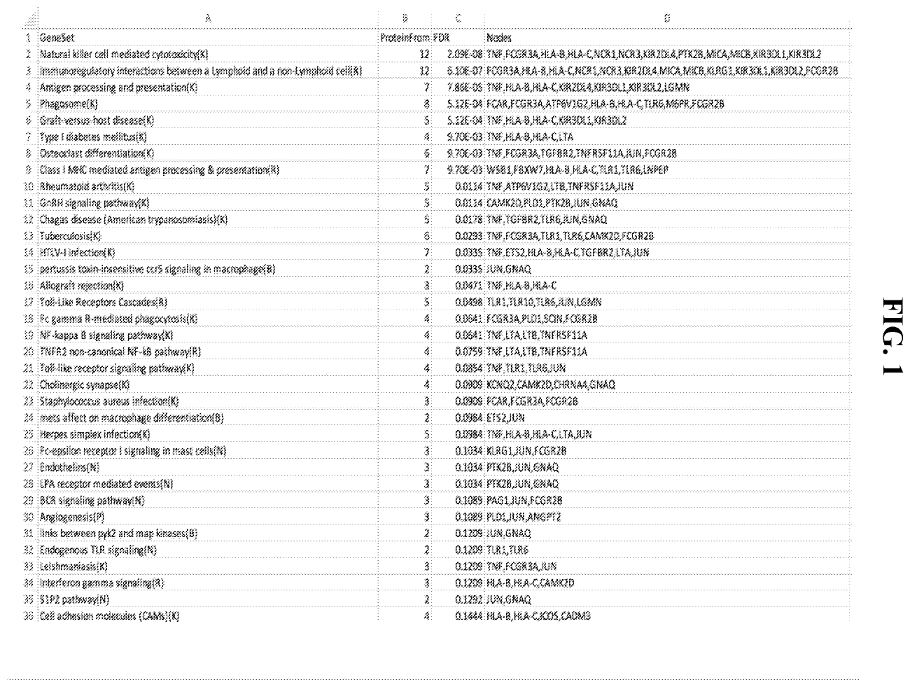

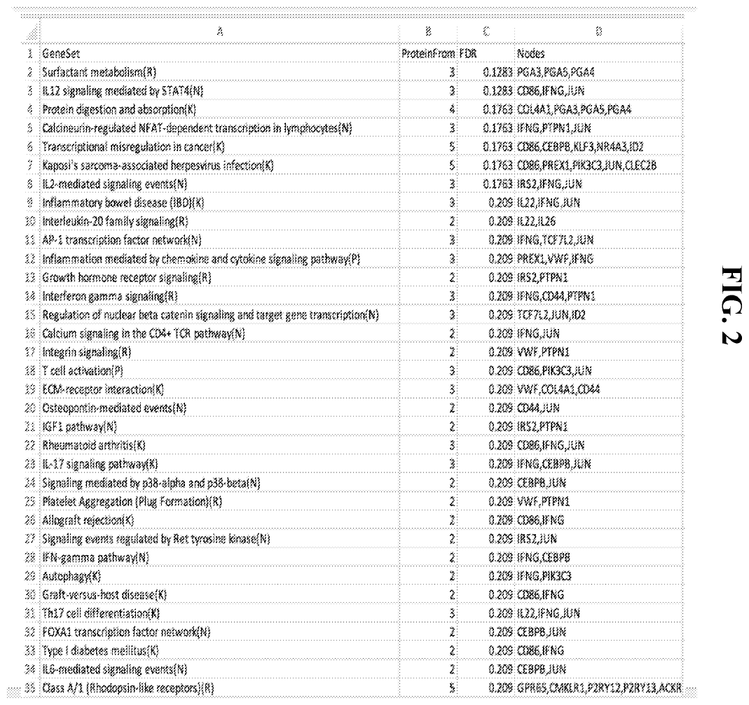

The chromosome interactions which are typed can be those mentioned in the subset shown in FIG. 1 or 2.

Any preferred number of chromosome interactions can be typed as mentioned herein from (i), (ii) or (iii), but typically at least 5, 8, 10, 12, 15, 20, 30 or 40 interactions will be typed.

Selecting a Subgroup with Particular Characteristics

This section provides examples of the number of interactions that can be typed from any one table.

The invention includes a process in which a specific combination of chromosome interactions are typed comprising at least 3, 5, 8, 10 or 20 of the chromosome interactions represented by the probes in Table 1. In one embodiment at least 10 chromosome interactions represented by the probes in Table 1 are typed.

The invention also includes a process in which specific combination of chromosome interactions are typed comprising at least 3, 5, 8, 10 or 20 of the chromosome interactions represented by the probes in Table 2. In one embodiment at least 10 chromosome interactions represented by the probes in Table 2 are typed.

The invention provides a process in which all of the chromosome interactions represented by the probes in Table 1 are typed. In certain embodiments at least 30, 40, 50, 60 or 80 of the chromosome interactions represented by the probes in Table 1 are typed. In particular embodiments at least 10, 20, 30, 50 or 80 chromosome interactions are typed which are present in a 4,000 base region which comprises or which flanks the chromosome interactions represented by the probes in Table 1.

The invention provides a process in which all of the chromosome interactions represented by the probes in Table 2 are typed. In certain embodiments at least 30, 40, 50, 60 or 80 of the chromosome interactions represented by the probes in Table 2 are typed. In particular embodiments at least 10, 20, 30, 50 or 80 chromosome interactions are typed which are present in a 4,000 base region which comprises or which flanks the chromosome interactions represented by the probes in Table 2.

The invention includes a process in which a specific combination of chromosome interactions are typed comprising at least 3, 5, 8, 10 or 20 of the chromosome interactions represented by the probes in Table 3. In one embodiment at least 10 chromosome interactions represented by the probes in Table 3 are typed.

The invention provides a process in which all of the chromosome interactions represented by the probes in Table 3 are typed. In certain embodiments at least 30, 40, 50, 60 or 80 of the chromosome interactions represented by the probes in Table 3 are typed. In particular embodiments at least 10, 20, 30, 50 or 80 chromosome interactions are typed which are present in a 4,000 base region which comprises or which flanks the chromosome interactions represented by the probes in Table 3.

The invention includes a process in which a specific combination of chromosome interactions are typed comprising at least 3, 5, 8, 10 or 20 of the chromosome interactions represented by the probes in Table 4. In one embodiment at least 10 chromosome interactions represented by the probes in Table 4 are typed.

The invention provides a process in which all of the chromosome interactions represented by the probes in Table 4 are typed. In certain embodiments at least 30, 40, 50, 60 or 80 of the chromosome interactions represented by the probes in Table 5 are typed. In particular embodiments at least 10, 20, 30, 50 or 80 chromosome interactions are typed which are present in a 4,000 base region which comprises or which flanks the chromosome interactions represented by the probes in Table 4.

The invention includes a process in which a specific combination of chromosome interactions are typed comprising at least 3, 5, 8, 10 or 20 of the chromosome interactions represented by the probes in Table 5. In one embodiment at least 10 chromosome interactions represented by the probes in Table 5 are typed.

The invention provides a process in which all of the chromosome interactions represented by the probes in Table 5 are typed. In certain embodiments at least 30, 40, 50, 60 or 80 of the chromosome interactions represented by the probes in Table 5 are typed. In particular embodiments at least 10, 20, 30, 50 or 80 chromosome interactions are typed which are present in a 4,000 base region which comprises or which flanks the chromosome interactions represented by the probes in Table 5.

The invention includes a process in which a specific combination of chromosome interactions are typed comprising at least 3, 5, 8, 10 or 20 of the chromosome interactions represented by the probes in Table 6. In one embodiment at least 10 chromosome interactions represented by the probes in Table 6 are typed.

The invention provides a process in which all of the chromosome interactions represented by the probes in Table 6 are typed. In certain embodiments at least 30, 40, 50, 60 or 80 of the chromosome interactions represented by the probes in Table 6 are typed. In particular embodiments at least 10, 20, 30, 50 or 80 chromosome interactions are typed which are present in a 4,000 base region which comprises or which flanks the chromosome interactions represented by the probes in Table 6.

The invention provides a process which comprises detecting the presence or absence of chromosome interactions, typically 5 to 20 or 5 to 500 such interactions, preferably 20 to 300 or 50 to 100 interactions, in order to determine the presence or absence of a characteristic relating to a subgroup. Preferably the chromosome interactions are those in any of the genes mentioned herein or in which chromosome interactions of the invention are present within.

In one aspect all of the interactions of Table 1 are typed. In one aspect all of the interactions of Table 2 are typed. In one aspect all of the interactions of Table 3 are typed. In one aspect all of the interactions of Table 4 are typed. In one aspect all of the interactions of Table 5 are typed. In one aspect all of the interactions of Table 6 are typed. In one aspect all of the interactions of Tables 3 and 4 are typed. In one aspect all of the interactions of Tables 5 and 6 are typed. In one aspect all of the interactions of Tables 3, 4, 5 and 6 are typed.

The detection of certain interactions shows presence of the relevant characteristic, whilst the presence of other interactions shows absence of the relevant characteristic as described in the tables.

Selecting Markers from Different Tables

The chromosome interactions which are typed can be selected from a single table or from more than one of Tables 1, 2, 3, 4, 5 or 6. Typically at least 5, 8, 10, 12, 15, 20, 25, 30, 40, 50, 60, 70, 80 interactions are typed. This number of interactions can be selected from 1, 2, 3, 4, 5 of or all of the Tables 1, 2, 3, 4, 5 or 6. In one aspect at least 5, 8, 10, 12, 15, 20 interactions to be typed are selected from all of Tables 3, 4, 5 and 6. In one aspect at least 3, 5, 8 or 10 interactions are selected from each of Tables 3 and 4. In one aspect at least 3, 5, 8 or 10 interactions are selected from each of Tables 5 and 6. In one aspect at least 10 interactions to be typed are selected from each of Tables 3 and 4. In one aspect at least 10 interactions to be typed are selected from each of Tables 5 and 6.

### Combinations of Markers Selected by SHAPLEY Analysis

A SHAPLEY analysis provides one way of measuring the performance of an individual marker. Tables 7 to 10 show the results of such modelling. In one aspect the markers to be typed can be selected based on these results, example based on any of the parameters which are shown. In a preferred aspect the chromosome interactions which are typed represent the top 10, 20, 30, 40, 50 or 60 markers selected based on such a parameter.

In a preferred aspect the ‘SHAPLEY_diff’ column is used to provide an indication of the power of a marker, and so for example the marker ORF1_chr6_31267448_31269252_31531115_31534994_RF_1 in Table 7 could be seen as the most powerful marker, given that it has the highest value for ‘SHAPLEY_diff’. Tables 7 to 10 list the markers in order of magnitude of ‘SHAPLEY_diff’, going from the most important to the less important as one goes down each table.

In one aspect at least 3 markers may be typed from the 5, 8, 10 or 20 markers shown at the top of Table 7. In one aspect at least 5 markers are typed from the 10 markers shown at the top of Table 7. In one aspect at least 5 markers may be typed from 20, 30, 40 or 50 markers shown at the top of Table 7. In one aspect at least 10 markers may be typed from the 30, 40 or 50 markers shown at the top of Table 7. In one aspect at least the 5, 8 or 10 markers shown at the top of Table 7 may be typed.

In one aspect at least 3 markers may be typed from the 5, 8, 10 or 20 markers shown at the top of Table 8. In one aspect at least 5 markers are typed from the 10 markers shown at the top of Table 8. In one aspect at least 5 markers may be typed from 20, 30, 40 or 50 markers shown at the top of Table 8. In one aspect at least 10 markers may be typed from the 30, 40 or 50 markers shown at the top of Table 8. In one aspect at least the 5, 8 or 10 markers shown at the top of Table 8 may be typed.

In one aspect at least 3 markers may be typed from the 5, 8, 10 or 20 markers shown at the top of Table 9. In one aspect at least 5 markers are typed from the 10 markers shown at the top of Table 9. In one aspect at least 5 markers may be typed from 20, 30, 40 or 50 markers shown at the top of Table 9. In one aspect at least 10 markers may be typed from the 30, 40 or 50 markers shown at the top of Table 9. In one aspect at least the 5, 8 or 10 markers shown at the top of Table 9 may be typed.

In one aspect at least 3 markers may be typed from the 5, 8, 10 or 20 markers shown at the top of Table 10. In one aspect at least 5 markers are typed from the 10 markers shown at the top of Table 10. In one aspect at least 5 markers may be typed from 20, 30, 40 or 50 markers shown at the top of Table 10. In one aspect at least 10 markers may be typed from the 30, 40 or 50 markers shown at the top of Table 10. In one aspect at least the 5, 8 or 10 markers shown at the top of Table 10 may be typed.

The Individual that is Tested

The individual that is tested in the process of the invention may have been selected in some way. The individual may be susceptible to any condition mentioned herein and/or may be in need of any therapy mentioned in. The individual may be receiving any therapy mentioned herein. In particular, the individual may have, or be suspected of having, fibrosis. The individual may have, or be suspected of having, a skin fibrotic condition, which may preferably be scleroderma.

The individual may have, or be suspected of having, a scleroderma condition that results in changes to the skin, blood vessels, muscles, and internal organs. The individual may have any one of the following symptoms: areas of thickened skin, stiffness, feeling tired, and poor blood flow to the fingers or toes with cold exposure. The individual may have, or be suspected of having, CREST syndrome, which may manifest as calcium deposits, Raynaud's syndrome, esophageal problems, thickening of the skin of the fingers and toes, and areas of small dilated blood vessels.

### Types of Chromosome Interaction

In one aspect the locus (including the gene and/or place where the chromosome interaction is detected) may comprise a CTCF binding site. This is any sequence capable of binding transcription repressor CTCF. That sequence may consist of or comprise the sequence CCCTC which may be present in 1, 2 or 3 copies at the locus. The CTCF binding site sequence may comprise the sequence CCGCGNGGNGGCAG (in IUPAC notation). The CTCF binding site may be within at least 100, 500, 1000 or 4000 bases of the chromosome interaction or within any of the chromosome regions shown Table 1 or 2. The CTCF binding site may be within at least 100, 500, 1000 or 4000 bases of the chromosome interaction or within any of the chromosome regions shown Table 3, 4, 5 or 6.

When detection is performed using a probe, typically sequence from both regions of the probe (i.e. from both sites of the chromosome interaction) could be detected. In preferred aspects probes are used in the process which comprise or consist of the same or complementary sequence to a probe shown in any table. In some aspects probes are used which comprise sequence which is homologous to any of the probe sequences shown in the tables.

### Tables Provided Herein

Tables 1 to 6 show specific markers represented by probes and relevant data. The probe sequences show sequence which can be used to detect a ligated product generated from both sites of gene regions that have come together in chromosome interactions, i.e. the probe will comprise sequence which is complementary to sequence in the ligated product. The first two sets of Start-End positions show probe positions, and the second two sets of Start-End positions show the relevant 4 kb region.

Table 1 shows 100 markers relating to early or late fibrosis (scleroderma), with 50 markers corresponding to each. Table 2 shows 100 markers relating to presence of fibrosis (scleroderma), with 50 associated with disease and 50 associated with absence of disease.

Table 3 shows 100 markers associated with early fibrosis (scleroderma).

Table 4 shows 100 markers associated with late fibrosis (scleroderma).

Table 5 shows 100 markers associated with presence of fibrosis (scleroderma).

Table 6 shows 100 markers associated with the absence of fibrosis (scleroderma).

The following information is provided in the probe data table:

RP—Rsum the Rank Product statistics evaluated per each chromosome interaction

FC—Interaction frequency (positive or negative)

Pfp—estimated percentage of false positive predictions (pfp), both considering positive and negative chromosome interactions

Pval—estimated pvalues per each CCSs being positive and negative

Adj.P.value (FDR)—False discovery rate adjusted p.value

Loop Detected—which state the loop is found in

Simple permutation-based estimation is used to determine how likely a given RP value or better is observed in a random experiment. This has the following steps:

1. Generate p permutations of k rank lists of length n.

2. Calculate the rank products of the n CCS in the p permutations.

3. Count (c) how many times the rank products of the CCS in the permutations are smaller or equal to the observed rank product. Set c to this value.

4. Calculate the average expected value for the rank product by: Erp (g)=c/p.

5. Calculate the percentage of false positives as: pfp (g)=Erp (g)/rank (g) where rank (g) is the rank of CCS g in a list of all n CCSs sorted by increasing RP.

The rank product statistic ranks chromosome interactions according to intensities within each microarray and calculates the product of these ranks across multiple microarrays. This technique can identify chromosome interactions that are consistently detected among the most differential chromosome interactions in a number of replicated microarrays. Where the p-value is 0 this indicates that there is very little variation in the Rank Product of the CCS across the samples, this is a good example of the signal to noise and effect size of CCS. Where p value is 0 and pfp is 0 this means that permutated Rank Product doesn't differ from the actual observed Rank Product. These methods are described Breitling R and Herzyk P (2005) Rank-based methods as a non-parametric alternative of the t-test for the analysis of biological microarray data. J Bioinf Comp Biol 3, 1171-1189.

The FC indicates prevalence of marker in each comparison, 2 means twice over average test, 1.5 means 1.5 over the average test, etc., and so FC indicates the weight of a marker to phenotype/group. The FC value can be used to give an indication of how many markers are needed for a highly effective test.

The probes are designed to be 30 bp away from the Taq1 site. In case of PCR, PCR primers are typically designed to detect ligated product but their locations from the Taq1 site vary. Probe locations:

Start 1—30 bases upstream of Taql site on fragment 1

End 1—Taql restriction site on fragment 1

Start 2—Taql restriction site on fragment 2

End 2—30 bases downstream of Taql site on fragment 2

4 kb Sequence Location:

Start 1—4000 bases upstream of Taql site on fragment 1

End 1—Taql restriction site on fragment 1

Start 2—Taql restriction site on fragment 2

End 2—4000 bases downstream of Taql site on fragment 2

SHAPLEY analysis can show the importance of each marker. It related to the marginal contribution of the marker to the Random Forest Model. The Shapley value is a solution concept in cooperative game theory applied to Machine learning to explain the contribution of markers to models Table 7 shows SHAPLEY results for markers associated with early fibrosis.

Table 8 shows SHAPLEY results for markers associated with late fibrosis.

Table 9 shows SHAPLEY results for markers associated with presence of fibrosis.

Table 10 shows SHAPLEY results for markers associated with absence of fibrosis.

In these tables the following information is provided:

HC_SHAPLEY—Health Control Class SHAPLEY value

SSc_SHAPLEY—SSc Class SHAPLEY value

E_SHAPLEY—Early Class SHAPLEY value

L_SHAPLEY—Late Class SHAPLEY value

SHAPLEY_diff—Difference between the two classes SHAPLEY Values

RPs_class1_class2—Rank Product Difference

RPrank_class1_class2—Rank Product Rank

pfp_class1_class2—percentage of false predictions

pval_class1_class2—Rank Product Pvalue

LogFC—Log Abundance Difference

FC—Linear Abundance Difference

### The Approach Taken to Identify Markers and Panels of Markers

The invention described herein relates to chromosome conformation profile and 3D architecture as a regulatory modality in its own right, closely linked to the phenotype. The discovery of biomarkers was based on annotations through pattern recognition and screening on representative cohorts of clinical samples representing the differences in phenotypes. We annotated and screened significant parts of the genome, across coding and non-coding parts and over large sways of non-coding 5′ and 3′ of known genes for identification of statistically disseminating consistent conditional disseminating chromosome conformations, which for example anchor in the non-coding sites within (intronic) or outside of open reading frames.

In selection of the best markers we are driven by statistical data and p values for the marker leads. Selected and validated chromosome conformations within the signature are disseminating stratifying entities in their own right, irrespective of the expression profiles of the genes used in the reference. Further work may be done on relevant regulatory modalities, such as SNPs at the anchoring sites, changes in gene transcription profiles, changes at the level of H3K27ac.

We are taking the question of clinical phenotype differences and their stratification from the basis of fundamental biology and epigenetics controls over phenotype—including for example from the framework of network of regulation. As such, to assist stratification, one can capture changes in the network and it is preferably done through signatures of several biomarkers, for example through following a machine learning algorithm for marker reduction which includes evaluating the optimal number of markers to stratify the testing cohort with minimal noise. This may end with 3-20 markers. Selection of markers for panels may be done by cross-validation statistical performance (and not for example by the functional relevance of the neighbouring genes, used for the reference name).

A panel of markers (with names of adjacent genes) is a product of clustered selection from the screening across significant parts of the genome, in non-biased way analysing statistical disseminating powers over 14,000-60,000 annotated EpiSwitch sites across significant parts of the genome. It should not be perceived as a tailored capture of a chromosome conformation on the gene of know functional value for the question of stratification. The total number of sites for chromosome interaction are 1.2 million, and so the potential number of combinations is 1.2 million to the power 1.2 million. The approach that we have followed nevertheless allows the identifying of the relevant chromosome interactions.

The specific markers that are provided by this application have passed selection, being statistically (significantly) associated with the condition. This is what the data in the relevant table demonstrates. Each marker can be seen as representing an event of biological epigenetic as part of network deregulation that is manifested in the relevant condition. In practical terms it means that these markers are prevalent across groups of patients when compared to controls. On average, as an example, an individual marker may typically be present in 80% of patients tested and in 10% of controls tested.

Simple addition of all markers would not represent the network interrelationships between some of the deregulations. This is where the standard multivariate biomarker analysis GLMNET (R package) is brought in. GLMNET package helps to identify interdependence between some of the markers, that reflect their joint role in achieving deregulations leading to disease phenotype. Modelling and then testing markers with highest GLMNET scores offers not only identify the minimal number of markers that accurately identifies the patient cohort, but also the minimal number that offers the least false positive results in the control group of patients, due to background statistical noise of low prevalence in the control group. Typically a group (combination) of selected markers (such as 3 to 10) offers the best balance between both sensitivity and specificity of detection, emerging in the context of multivariate analysis from individual properties of all the selected statistical significant markers for the condition.

The tables herein show the reference names for the array probes (60-mer) for array analysis that overlaps the juncture between the long range interaction sites, the chromosome number and the start and end of two chromosomal fragments that come into juxtaposition.

### Preferred Aspects for Sample Preparation and Chromosome Interaction Detection

Methods of preparing samples and detecting chromosome conformations are described herein. Optimised (non-conventional) versions of these processes can be used, for example as described in this section.

Typically the sample will contain at least 2×10⁵ cells. The sample may contain up to 5×10⁵ cells. In one aspect, the sample will contain 2×10⁵ to 5.5×10⁵ cells

Crosslinking of epigenetic chromosomal interactions present at the chromosomal locus is described herein. This may be performed before cell lysis takes place. Cell lysis may be performed for 3 to 7 minutes, such as 4 to 6 or about 5 minutes. In some aspects, cell lysis is performed for at least 5 minutes and for less than 10 minutes.

Digesting DNA with a restriction enzyme is described herein. Typically, DNA restriction is performed at about 55° C. to about 70° C., such as for about 65° C., for a period of about 10 to 30 minutes, such as about 20 minutes.

Preferably a frequent cutter restriction enzyme is used which results in fragments of ligated DNA with an average fragment size up to 4000 base pair. Optionally the restriction enzyme results in fragments of ligated DNA have an average fragment size of about 200 to 300 base pairs, such as about 256 base pairs.

In one aspect, the typical fragment size is from 200 base pairs to 4,000 base pairs, such as 400 to 2,000 or 500 to 1,000 base pairs.

In one aspect of the EpiSwitch process a DNA precipitation step is not performed between the DNA restriction digest step and the DNA ligation step.

DNA ligation is described herein. Typically the DNA ligation is performed for 5 to 30 minutes, such as about 10 minutes.

The protein in the sample may be digested enzymatically, for example using a proteinase, optionally Proteinase K. The protein may be enzymatically digested for a period of about 30 minutes to 1 hour, for example for about 45 minutes. In one aspect after digestion of the protein, for example Proteinase K digestion, there is no cross-link reversal or phenol DNA extraction step.

In one aspect PCR detection is capable of detecting a single copy of the ligated nucleic acid, preferably with a binary read-out for presence/absence of the ligated nucleic acid.

FIG. 3 shows a preferred process of detecting chromosome interactions.

### Processes and Uses of the Invention

The process of the invention can be described in different ways. It can be described as a process of making a ligated nucleic acid comprising (i) in vitro cross-linking of chromosome regions which have come together in a chromosome interaction; (ii) subjecting said cross-linked DNA to cutting or restriction digestion cleavage; and (iii) ligating said cross-linked cleaved DNA ends to form a ligated nucleic acid, wherein detection of the ligated nucleic acid may be used to determine the chromosome state at a locus, and wherein preferably:
the locus may be any of the loci or regions mentioned in Table 1, 2, 3, 4, 5 or 6, and/or
wherein the chromosomal interaction may be any of the chromosome interactions mentioned herein or corresponding to any of the probes disclosed in Table 1, 2, 3, 4, 5 or 6, and/or
wherein the ligated product may have or comprise (i) sequence which is the same as or homologous to any of the probe sequences disclosed in Table 1, 2, 3, 4, 5 or 6; or (ii) sequence which is complementary to (ii).

The process of the invention can be described as a process for detecting chromosome states which represent different subgroups in a population comprising determining whether a chromosome interaction is present or absent within a defined epigenetically active region of the genome, wherein preferably:
the subgroup is defined by presence or stage of fibrosis, and/or
the chromosome state may be at any locus or region mentioned in Table 1, 2, 3, 4, 5 or 6; and/or
the chromosome interaction may be any of those mentioned in Table 1, 2, 3, 4, 5 or 6, or corresponding to any of the probes disclosed in those tables.

### Use of the Process of the Invention to Identify New Treatments

Knowledge of chromosome interactions can be used to identify new treatments for conditions. The invention provides processes and uses of chromosome interactions defined herein to identify or design new therapeutic agents, for example relating to therapy of fibrosis or related sub-conditions.

### Homologues

Homologues of polynucleotide/nucleic acid (e.g. DNA) sequences are referred to herein. Such homologues typically have at least 70% homology, preferably at least 80%, at least 85%, at least 90%, at least 95%, at least 97%, at least 98% or at least 99% homology, for example over a region of at least 10, 15, 20, 30, 100 or more contiguous nucleotides, or across the portion of the nucleic acid which is from the region of the chromosome involved in the chromosome interaction. The homology may be calculated on the basis of nucleotide identity (sometimes referred to as “hard homology”).

Therefore, in a particular aspect, homologues of polynucleotide/nucleic acid (e.g. DNA) sequences are referred to herein by reference to percentage sequence identity. Typically such homologues have at least 70% sequence identity, preferably at least 80%, at least 85%, at least 90%, at least 95%, at least 97%, at least 98% or at least 99% sequence identity, for example over a region of at least 10, 15, 20, 30, 100 or more contiguous nucleotides, or across the portion of the nucleic acid which is from the region of the chromosome involved in the chromosome interaction.

For example the UWGCG Package provides the BESTFIT program which can be used to calculate homology and/or % sequence identity (for example used on its default settings) (Devereux et al (1984) Nucleic Acids Research 12, p387-395). The PILEUP and BLAST algorithms can be used to calculate homology and/or % sequence identity and/or line up sequences (such as identifying equivalent or corresponding sequences (typically on their default settings)), for example as described in Altschul S. F. (1993) J Mol Evol 36:290-300; Altschul, S, F et al (1990) J Mol Biol 215:403-10.

Software for performing BLAST analyses is publicly available through the National Center for Biotechnology Information. This algorithm involves first identifying high scoring sequence pair (HSPs) by identifying short words of length W in the query sequence that either match or satisfy some positive-valued threshold score T when aligned with a word of the same length in a database sequence. T is referred to as the neighbourhood word score threshold (Altschul et al, supra). These initial neighbourhood word hits act as seeds for initiating searches to find HSPs containing them. The word hits are extended in both directions along each sequence for as far as the cumulative alignment score can be increased. Extensions for the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W5 T and X determine the sensitivity and speed of the alignment. The BLAST program uses as defaults a word length (W) of 11, the BLOSUM62 scoring matrix (see Henikoff and Henikoff (1992) Proc. Natl. Acad. Sci. USA 89: 10915-10919) alignments (B) of 50, expectation (E) of 10, M=5, N=4, and a comparison of both strands.

The BLAST algorithm performs a statistical analysis of the similarity between two sequences; see e.g., Karlin and Altschul (1993) Proc. Natl. Acad. Sci. USA 90: 5873-5787. One measure of similarity provided by the BLAST algorithm is the smallest sum probability (P(N)), which provides an indication of the probability by which a match between two polynucleotide sequences would occur by chance. For example, a sequence is considered similar to another sequence if the smallest sum probability in comparison of the first sequence to the second sequence is less than about 1, preferably less than about 0.1, more preferably less than about 0.01, and most preferably less than about 0.001.

The homologous sequence typically differs by 1, 2, 3, 4 or more bases, such as less than 10, 15 or 20 bases (which may be substitutions, deletions or insertions of nucleotides). These changes may be measured across any of the regions mentioned above in relation to calculating homology and/or % percentage sequence identity.

Homology of a ‘pair of primers’ can be calculated, for example, by considering the two sequences as a single sequence (as if the two sequences are joined together) for the purpose of then comparing against the another primer pair which again is considered as a single sequence.

### Arrays

The second set of nucleic acids may be bound to an array, and in one aspect there are at least 15,000, 45,000, 100,000 or 250,000 different second nucleic acids bound to the array, which preferably represent at least 300, 900, 2000 or 5000 loci. In one aspect one, or more, or all of the different populations of second nucleic acids are bound to more than one distinct region of the array, in effect repeated on the array allowing for error detection. The array may be based on an Agilent SurePrint G3 Custom CGH microarray platform. Detection of binding of first nucleic acids to the array may be performed by a dual colour system.

### Therapeutic Agents

This section is relevant both to:
therapeutic agents which are given to individuals selected by the process of the invention, and
therapeutic agents which are selected based on the results of the process of the invention.

The invention provides therapeutic agents for use in preventing or treating a disease condition in certain individuals, for example those identified by a process of the invention. This may comprise administering to an individual in need a therapeutically effective amount of the agent. The invention provides use of the agent in the manufacture of a medicament to prevent or treat a condition in certain individuals. The disease or condition may be fibrosis, any type of fibrosis sub-condition or a stage of fibrosis.

The formulation of the agent will depend upon the nature of the agent. The agent will be provided in the form of a pharmaceutical composition containing the agent and a pharmaceutically acceptable carrier or diluent. Suitable carriers and diluents include isotonic saline solutions, for example phosphate-buffered saline. Typical oral dosage compositions include tablets, capsules, liquid solutions and liquid suspensions. The agent may be formulated for parenteral, intravenous, intramuscular, subcutaneous, transdermal or oral administration.

The dose of an agent may be determined according to various parameters, especially according to the substance used; the age, weight and condition of the individual to be treated; the route of administration; and the required regimen. A physician will be able to determine the required route of administration and dosage for any particular agent. A suitable dose may however be from 0.1 to 100 mg/kg body weight such as 1 to 40 mg/kg body weight, for example, to be taken from 1 to 3 times daily.

The therapeutic agent may be any such agent disclosed herein, or may target any ‘target’ disclosed herein, including any protein or gene disclosed herein in any table. It is understood that any agent that is disclosed in a combination should be seen as also disclosed for administration individually.

Therapeutic agents which can be used in the invention include:
inflammation therapy, such as NSAIDs (e.g. ibuprofen) or corticosteroids (e.g. prednisone);
immunosuppressive therapy, such astocilizumab, methotrexate, cyclosporine, antithymocyte globulin, mycophenolate mofetil or cyclophosphamide;
vascular therapy, such as nifedipine, renin, endothelin, prostaglandins and nitric oxide, bosentan, epoprostenol (prostacyclin) or aspirin;
anti-fibrotic agents, such as colchicine, para-aminobenzoic acid (PABA), dimethyl sulfoxide or D-penicillamine.

The therapeutic agent may be selected from the following:
lenabasum
IL4/IL13 combination
immunoglobin, preferably delivered intravenously
ROCK inhibitor
pirfenidone with MMF
oncostatin M
putotaxin inhibitor
brentuximab vedotin
teprotumumab
a TGF beta trap, preferably a TGF beta 1,3 trap.

Astocilizumab is a preferred therapeutic agent.

### Forms of the Substance Mentioned Herein

Any of the substances, such as nucleic acids or therapeutic agents, mentioned herein may be in purified or isolated form. They may be in a form which is different from that found in nature, for example they may be present in combination with other substance with which they do not occur in nature. The nucleic acids (including portions of sequences defined herein) may have sequences which are different to those found in nature, for example having at least 1, 2, 3, 4 or more nucleotide changes in the sequence as described in the section on homology. The nucleic acids may have heterologous sequence at the 5′ or 3′ end. The nucleic acids may be chemically different from those found in nature, for example they may be modified in some way, but preferably are still capable of Watson-Crick base pairing. Where appropriate the nucleic acids will be provided in double stranded or single stranded form. The invention provides all of the specific nucleic acid sequences mentioned herein in single or double stranded form, and thus includes the complementary strand to any sequence which is disclosed.

The invention provides a kit for carrying out any process of the invention, including detection of a chromosomal interaction relating to prognosis. Such a kit can include a specific binding agent capable of detecting the relevant chromosomal interaction, such as agents capable of detecting a ligated nucleic acid generated by processes of the invention. Preferred agents present in the kit include probes capable of hybridising to the ligated nucleic acid or primer pairs, for example as described herein, capable of amplifying the ligated nucleic acid in a PCR reaction. A kit of the invention may comprise means to detect a panel of markers, such as any number of combination of markers disclosed herein.

The invention provides use of a reagent for preparing kit for carrying out the process of the invention. Such a reagent may be any suitable substance mentioned herein, such as the agents which are capable of detection of products of detection processes, including reagents which are any of the probes or primers mentioned herein. The invention provides use of the reagent in the process of the invention. The invention provides use of the reagent in the preparing of a means for carrying out the invention. The invention also provides a process for detecting a characteristic of fibrosis, including any type of fibrosis mentioned here and including detecting the stage of fibrosis or the presence of fibrosis.

The invention provides a method of obtaining diagnostic information relating to fibrosis, such as the stage and/or presence of fibrosis. The invention provides a method of detecting a chromosome state defined by any number or combination of chromosome interactions disclosed herein. The invention provides a method of detecting a specific pattern of chromosome interactions, for example as defined by any number or combination of chromosome interactions disclosed herein.

The invention provides a device that is capable of detecting the relevant chromosome interactions. The device preferably comprises any specific binding agents, probe or primer pair capable of detecting the chromosome interaction, such as any such agent, probe or primer pair described herein.

The invention provides use of detection of chromosome interactions as defined herein (for example by number or specific combination) to detect a characteristic of fibrosis, for example as defined herein. The invention provides use of a reagent (for example a probe, primer, label, device or array) in any method of the invention.

### The Threshold of Detection

The markers which are disclosed herein have been found to be ‘disseminating markers’ capable of determining the relevant subgroup and Tables 1 to 6 show which subgroup each marker is present in.

In practical terms it means that these markers are prevalent across the relevant subgroup when compared to controls (as is shown by the FC value, for example). On average, as an example, an individual marker may typically be present in 80% of the relevant subgroup and in 10% of controls (for the subgroup). When testing an individual the result will be a combination of ‘present’ and ‘absent’ chromosome interactions for each of the markers shown in Tables 1 to 6 allowing determination of the subgroup for the individual. Typically presence/absence of at least 8 markers out of 10 compared to the ‘ideal’ result shown in the table can be used to assign the individual to a subgroup.

### Types of Method

The invention relates to markers which are effective detecting a characteristic relating to fibrosis as described herein. Clearly different numbers of markers can be used as the basis of a test. In one aspect the invention provides a process for detecting a chromosome state which represents a subgroup in a population comprising determining whether a chromosome interaction relating to that chromosome state is present or absent within a defined region of the genome; wherein:

(i) one or more chromosome interactions are typed which are associated with early stage fibrosis from Tables 1 and 3 and if at least 70% or 80% of these are present the individual is classed as have early stage fibrosis, and/or

(ii) one or more chromosome interactions are typed which are associated with early stage fibrosis from Tables 1 and 3 and if at least 70% or 80% of these are absent the individual is classed as not having early stage fibrosis, and/or

(iii) one or more chromosome interactions are typed which are associated with late stage fibrosis from Tables 1 and 4 and if at least 70% or 80% of these are present the individual is classed as have late stage fibrosis, and/or

(iv) one or more chromosome interactions are typed which are associated with late stage fibrosis from Tables 1 and 4 and if at least 70% or 80% of these are absent the individual is classed as not having late stage fibrosis, and/or

(v) one or more chromosome interactions are typed which are associated with presence of fibrosis from Tables 2 and 5 and if at least 70% or 80% of these are present the individual is classed as having fibrosis, and/or

(vi) one or more chromosome interactions are typed which are associated with presence of fibrosis from Tables 2 and 5 and if at least 70% or 80% of these are absent the individual is classed as not having fibrosis, and/or

(vii) one or more chromosome interactions are typed which are associated with absence of fibrosis from Tables 2 and 6 and if at least 70% or 80% of these are present the individual is classed as having absence of fibrosis, and/or

(viii) one or more chromosome interactions are typed which are associated with absence of fibrosis from Tables 2 and 6 and if at least 70% or 80% of these are absent the individual is classed as having fibrosis.

In this aspect any number and combination of markers may be typed, for example as disclosed herein.

### Detection Process

In one aspect quantitative detection of the ligated sequence which is relevant to a chromosome interaction is carried out using a probe which is detectable upon activation during a PCR reaction, wherein said ligated sequence comprises sequences from two chromosome regions that come together in an epigenetic chromosome interaction, wherein said process comprises contacting the ligated sequence with the probe during a PCR reaction, and detecting the extent of activation of the probe, and wherein said probe binds the ligation site. The process typically allows particular interactions to be detected in a MIQE compliant manner using a dual labelled fluorescent hydrolysis probe.

The probe is generally labelled with a detectable label which has an inactive and active state, so that it is only detected when activated. The extent of activation will be related to the extent of template (ligation product) present in the PCR reaction. Detection may be carried out during all or some of the PCR, for example for at least 50% or 80% of the cycles of the PCR.

The probe can comprise a fluorophore covalently attached to one end of the oligonucleotide, and a quencher attached to the other end of the nucleotide, so that the fluorescence of the fluorophore is quenched by the quencher. In one aspect the fluorophore is attached to the 5′end of the oligonucleotide, and the quencher is covalently attached to the 3′ end of the oligonucleotide. Fluorophores that can be used in the process of the invention include FAM, TET, JOE, Yakima Yellow, HEX, Cyanine3, ATTO 550, TAMRA, ROX, Texas Red, Cyanine 3.5, LC610, LC640, ATTO 647N, Cyanine 5, Cyanine 5.5 and ATTO 680. Quenchers that can be used with the appropriate fluorophore include TAM, BHQ1, DAB, Eclip, BHQ2 and BBQ650, optionally wherein said fluorophore is selected from HEX, Texas Red and FAM. Preferred combinations of fluorophore and quencher include FAM with BHQ1 and Texas Red with BHQ2.

Use of the Probe in a qPCR Assay

Hydrolysis probes of the invention are typically temperature gradient optimised with concentration matched negative controls. Preferably single-step PCR reactions are optimized. More preferably a standard curve is calculated. An advantage of using a specific probe that binds across the junction of the ligated sequence is that specificity for the ligated sequence can be achieved without using a nested PCR approach. The processes described herein allow accurate and precise quantification of low copy number targets. The target ligated sequence can be purified, for example gel-purified, prior to temperature gradient optimization. The target ligated sequence can be sequenced. Preferably PCR reactions are performed using about 10 ng, or 5 to 15 ng, or 10 to 20 ng, or 10 to 50 ng, or 10 to 200 ng template DNA. Forward and reverse primers are designed such that one primer binds to the sequence of one of the chromosome regions represented in the ligated DNA sequence, and the other primer binds to other chromosome region represented in the ligated DNA sequence, for example, by being complementary to the sequence.

### Choice of Ligated DNA Target

The invention includes selecting primers and a probe for use in a PCR process as defined herein comprising selecting primers based on their ability to bind and amplify the ligated sequence and selecting the probe sequence based properties of the target sequence to which it will bind, in particular the curvature of the target sequence.

Probes are typically designed/chosen to bind to ligated sequences which are juxtaposed restriction fragments spanning the restriction site. In one aspect of the invention, the predicted curvature of possible ligated sequences relevant to a particular chromosome interaction is calculated, for example using a specific algorithm referenced herein. The curvature can be expressed as degrees per helical turn, e.g. 10.5° per helical turn. Ligated sequences are selected for targeting where the ligated sequence has a curvature propensity peak score of at least 5° per helical turn, typically at least 10°, 15° or 20° per helical turn, for example 5° to 20° per helical turn. Preferably the curvature propensity score per helical turn is calculated for at least 20, 50, 100, 200 or 400 bases, such as for 20 to 400 bases upstream and/or downstream of the ligation site. Thus in one aspect the target sequence in the ligated product has any of these levels of curvature. Target sequences can also be chosen based on lowest thermodynamic structure free energy.

### Particular Aspects

In one aspect only intrachromosomal interactions are typed/detected, and no extrachromosomal interactions (between different chromosomes) are typed/detected.

In particular aspects certain chromosome interactions are not typed, for example any specific interaction mentioned herein (for example as defined by any probe or primer pair mentioned herein). In some aspects chromosome interactions are not typed in any of the genes relevant to chromosome interactions mentioned herein.

The data provided herein shows that the markers are ‘disseminating’ ones able to differentiate cases and non-cases for the relevant disease situation. Therefore when carrying out the invention the skilled person will be able to determine by detection of the interactions which subgroup the individual is in. In one embodiment a threshold value of detection of at least 70% of the tested markers in the form they are associated with the relevant subgroup situation may be used to determine whether the individual is in the relevant subgroup.

In one aspect any single marker disclosed in any table might be typed together any other number or combination of markers disclosed herein (for example from the same table or one or more other tables).

In one aspect 1, 2, 3, 4, or more groups of markers may be typed which are each a number or combination of markers disclosed herein.

In one aspect 200 or less markers are typed, for example 100 or less markers, 50 or less, 25 or less, or 20 or less markers are typed. In one aspect 200 or less of the specific markers shown in the tables are typed, for example 100 or less, 50 or less, 25 or less, or 20 or less such markers are typed.

### Screening Process

The invention provides a process of determining which chromosomal interactions are relevant to a chromosome state corresponding to an prognosis subgroup of the population, comprising contacting a first set of nucleic acids from subgroups with different states of the chromosome with a second set of index nucleic acids, and allowing complementary sequences to hybridise, wherein the nucleic acids in the first and second sets of nucleic acids represent a ligated product comprising sequences from both the chromosome regions that have come together in chromosomal interactions, and wherein the pattern of hybridisation between the first and second set of nucleic acids allows a determination of which chromosomal interactions are specific to an prognosis subgroup. The subgroup may be any of the specific subgroups defined herein, for example with reference to particular conditions or therapies.

### Publications

The contents of all publications mentioned herein are incorporated by reference into the present specification and may be used to further define the features relevant to the invention. The contents of all priority applications are incorporated by reference into the present specification and may be used to define the features relevant to the invention.

### Use of a Classifier

The method of the invention may include analysis of the chromosome interactions identified in the individual, for example using a classifier, which may increase performance, such as sensitivity or specificity. The classifier is typically one that has been ‘trained’ on samples from the population and such training may assist the classifier to detect any subgroup mentioned herein.

### Specific Embodiments

Certain embodiments will be described with reference to numbered paragraphs:

Paragraph 1. A process for detecting a chromosome state which represents a subgroup in a population comprising determining whether a chromosome interaction relating to that chromosome state is present or absent within a defined region of the genome; and
wherein said chromosome interaction has optionally been identified by a process of determining which chromosomal interactions are relevant to a chromosome state corresponding to the subgroup of the population, comprising contacting a first set of nucleic acids from subgroups with different states of the chromosome with a second set of index nucleic acids, and allowing complementary sequences to hybridise, wherein the nucleic acids in the first and second sets of nucleic acids represent a ligated product comprising sequences from both the chromosome regions that have come together in chromosomal interactions, and wherein the pattern of hybridisation between the first and second set of nucleic acids allows a determination of which chromosomal interactions are specific to the subgroup; and
wherein the subgroup relates to the stage of fibrosis and the chromosome interaction either:

(i) is present in any one of the regions or genes listed in Table 1; and/or

(ii) corresponds to any one of the chromosome interactions represented by any probe shown in Table 1; and/or

(iii) is present in a 4,000 base region which comprises or which flanks (i) or (ii);

or
wherein the subgroup relates to the presence of fibrosis and the chromosome interaction either:

(a) is present in any one of the regions or genes listed in Table 2; and/or

(b) corresponds to any one of the chromosome interactions represented by any probe shown in Table 2, and/or

(c) is present in a 4,000 base region which comprises or which flanks (a) or (b).

Paragraph 2. A process according to Paragraph 1 wherein the subgroup relates to the stage of sclerosis or the presence of sclerosis.

Paragraph 3. A process according to Paragraph 1 or 2 wherein a specific combination of chromosome interactions are typed comprising at least 3, 5, 8, 10 or 20 of the chromosome interactions represented by the probes in Table 1.

Paragraph 4. A process according to any one of the preceding Paragraphs wherein a specific combination of chromosome interactions are typed comprising at least 3, 5, 8, 10 or 20 of the chromosome interactions represented by the probes in Table 2.

Paragraph 5. A process according to any one of the preceding Paragraphs which is carried out to detect the whether the fibrosis is early stage or late stage fibrosis.

Paragraph 6. A process according to any one of the preceding Paragraphs which is carried out to determine the stage of fibrosis in an individual and wherein at least 10 chromosome interactions represented by the probes of Table 1 are typed.

Paragraph 7. A process according to any one of Paragraphs 1 to 4 which is carried out to determine the stage of presence of fibrosis in an individual and wherein at least 10 chromosome interactions represented by the probes of Table 2 are typed.

Paragraph 8. A process according to any one of the preceding Paragraphs in which the chromosome interactions are typed:
in a sample from an individual, and/or
by detecting the presence or absence of a DNA loop at the site of the chromosome interactions, and/or
detecting the presence or absence of distal regions of a chromosome being brought together in a chromosome conformation, and/or
by detecting the presence of a ligated nucleic acid which is generated during said typing and whose sequence comprises two regions each corresponding to the regions of the chromosome which come together in the chromosome interaction, wherein detection of the ligated nucleic acid is preferably by:

(i) by a probe that has at least 70% identity to any of the specific probe sequences mentioned in Table 1 or 2, and/or

(ii) by a primer pair which has at least 70% identity to any primer pair in Table 1 or 2.

Paragraph 9. A process according to any one of the preceding Paragraphs, wherein:
the second set of nucleic acids is from a larger group of individuals than the first set of nucleic acids; and/or
the first set of nucleic acids is from at least 8 individuals; and/or
the first set of nucleic acids is from at least 4 individuals from a first subgroup and at least 4 individuals from a second subgroup which is preferably non-overlapping with the first subgroup.

Paragraph 10. A process according to any one of the preceding Paragraphs wherein:
the second set of nucleic acids represents an unselected group; and/or
wherein the second set of nucleic acids is bound to an array at defined locations; and/or
wherein the second set of nucleic acids represents chromosome interactions in least 100 different genes; and/or
wherein the second set of nucleic acids comprises at least 1,000 different nucleic acids representing at least 1,000 different chromosome interactions; and/or
wherein the first set of nucleic acids and the second set of nucleic acids comprise at least 100 nucleic acids with length 10 to 100 nucleotide bases.

Paragraph 11. A process according to any one of the preceding Paragraphs, wherein the first set of nucleic acids is obtainable in a process comprising the steps of:

(i) cross-linking of chromosome regions which have come together in a chromosome interaction;

(ii) subjecting said cross-linked regions to cleavage, optionally by restriction digestion cleavage with an enzyme; and

(iii) ligating said cross-linked cleaved DNA ends to form the first set of nucleic acids (in particular comprising ligated DNA).

Paragraph 12. A process according to any one of the preceding Paragraphs wherein said defined region of the genome:

(i) comprises a single nucleotide polymorphism (SNP); and/or

(ii) expresses a microRNA (miRNA); and/or

(iii) expresses a non-coding RNA (ncRNA); and/or

(iv) expresses a nucleic acid sequence encoding at least 10 contiguous amino acid residues; and/or

(v) expresses a regulating element; and/or

(vii) comprises a CTCF binding site.

Paragraph 13. A process according to any one of the preceding Paragraphs wherein:
the result of the process is provided in a report; and/or
the result of the process is used to select a treatment schedule; and/or
the result of the process is used to select a specific therapy for the individual; and/or
the process is carried out to select an individual for a medical treatment.

Paragraph 14. A process according to any one of Paragraphs 1 to 12 which is carried out to identify or design a therapeutic agent for fibrosis;
wherein preferably said process is used to detect whether a candidate agent is able to cause a change to a chromosome state which is associated with fibrosis;
wherein the chromosomal interaction is represented by any probe in Table 1 or 2; and/or
the chromosomal interaction is present in any region or gene listed in Table 1 or 2;

and wherein optionally:
the chromosomal interaction has been identified by the process of determining which chromosomal interactions are relevant to a subgroup as defined in Paragraph 1 or 2, and/or
the change in chromosomal interaction is monitored using (i) a probe that has at least 70% identity to any of the probe sequences mentioned in Table 1 or 2, and/or (ii) by a primer pair which has at least 70% identity to any primer pair in Table 1 or 2.

Paragraph 15. A process according to Paragraph 14 which comprises selecting a target based on detection of the chromosome interactions, and preferably screening for a modulator of the target to identify a therapeutic agent for fibrosis, wherein said target is optionally a protein.

Paragraph 16. A process according to any one of the preceding claims, wherein the typing or detecting comprises specific detection of the ligated product by quantitative PCR (qPCR) which uses primers capable of amplifying the ligated product and a probe which binds the ligation site during the PCR reaction, wherein said probe comprises sequence which is complementary to sequence from each of the chromosome regions that have come together in the chromosome interaction, wherein preferably said probe comprises:

an oligonucleotide which specifically binds to said ligated product, and/or

a fluorophore covalently attached to the 5′ end of the oligonucleotide, and/or

a quencher covalently attached to the 3′ end of the oligonucleotide, and

optionally

said fluorophore is selected from HEX, Texas Red and FAM; and/or

said probe comprises a nucleic acid sequence of length 10 to 40 nucleotide bases, preferably a length of 20 to 30 nucleotide bases.

Paragraph 17. A therapeutic agent for use in a method of treating fibrosis in an individual that has been identified as being in need of the therapeutic agent by a process according to any one of Paragraphs 1 to 12 and 16.

### Specific Aspects

The EpiSwitch™ platform technology detects epigenetic regulatory signatures of regulatory changes between normal and abnormal conditions at loci. The EpiSwitch™ platform identifies and monitors the fundamental epigenetic level of gene regulation associated with regulatory high order structures of human chromosomes also known as chromosome conformation signatures. Chromosome signatures are a distinct primary step in a cascade of gene deregulation. They are high order biomarkers with a unique set of advantages against biomarker platforms that utilize late epigenetic and gene expression biomarkers, such as DNA methylation and RNA profiling.

### EpiSwitch′ Array Assay

The custom EpiSwitch™ array-screening platforms come in 4 densities of, 15K, 45K, 100K, and 250K unique chromosome conformations, each chimeric fragment is repeated on the arrays 4 times, making the effective densities 60K, 180K, 400K and 1 Million respectively.

### Custom Designed EpiSwitch™ Arrays

The 15K EpiSwitch™ array can screen the whole genome including around 300 loci interrogated with the EpiSwitch™ Biomarker discovery technology. The EpiSwitch™ array is built on the Agilent SurePrint G3 Custom CGH microarray platform; this technology offers 4 densities, 60K, 180K, 400K and 1 Million probes. The density per array is reduced to 15K, 45K, 100K and 250K as each EpiSwitch™ probe is presented as a quadruplicate, thus allowing for statistical evaluation of the reproducibility. The average number of potential EpiSwitch™ markers interrogated per genetic loci is 50, as such the numbers of loci that can be investigated are 300, 900, 2000, and 5000.

### EpiSwitch′ Custom Array Pipeline

The EpiSwitch™ array is a dual colour system with one set of samples, after EpiSwitch™ library generation, labelled in Cy5 and the other of sample (controls) to be compared/analyzed labelled in Cy3. The arrays are scanned using the Agilent SureScan Scanner and the resultant features extracted using the Agilent Feature Extraction software. The data is then processed using the EpiSwitch™ array processing scripts in R. The arrays are processed using standard dual colour packages in Bioconductor in R: Limma*. The normalisation of the arrays is done using the normalisedWithinArrays function in Limma* and this is done to the on chip Agilent positive controls and EpiSwitch™ positive controls. The data is filtered based on the Agilent Flag calls, the Agilent control probes are removed and the technical replicate probes are averaged, in order for them to be analysed using Limma*. The probes are modelled based on their difference between the 2 scenarios being compared and then corrected by using False Discovery Rate. Probes with Coefficient of Variation (CV)<=30% that are <=−1.1 or =>1.1 and pass the p<=0.1 FDR p-value are used for further screening. To reduce the probe set further Multiple Factor Analysis is performed using the FactorMineR package in R.

*Note: LIMMA is Linear Models and Empirical Bayes Processes for Assessing Differential Expression in Microarray Experiments. Limma is an R package for the analysis of gene expression data arising from microarray or RNA-Seq.

The pool of probes is initially selected based on adjusted p-value, FC and CV<30% (arbitrary cut off point) parameters for final picking. Further analyses and the final list are drawn based only on the first two parameters (adj. p-value; FC).

### Statistical Pipeline

EpiSwitch™ screening arrays are processed using the EpiSwitch™ Analytical Package in R in order to select high value EpiSwitch™ markers for translation on to the EpiSwitch™ PCR platform.

### Step 1

Probes are selected based on their corrected p-value (False Discovery Rate, FDR), which is the product of a modified linear regression model. Probes below p-value <=0.1 are selected and then further reduced by their Epigenetic ratio (ER), probes ER have to be <=−1.1 or =>1.1 in order to be selected for further analysis. The last filter is a coefficient of variation (CV), probes have to be below <=0.3.

### Step 2

The top 40 markers from the statistical lists are selected based on their ER for selection as markers for PCR translation. The top 20 markers with the highest negative ER load and the top 20 markers with the highest positive ER load form the list.

### Step 3

The resultant markers from step 1, the statistically significant probes form the bases of enrichment analysis using hypergeometric enrichment (HE). This analysis enables marker reduction from the significant probe list, and along with the markers from step 2 forms the list of probes translated on to the EpiSwitch™ PCR platform.

The statistical probes are processed by HE to determine which genetic locations have an enrichment of statistically significant probes, indicating which genetic locations are hubs of epigenetic difference.

The most significant enriched loci based on a corrected p-value are selected for probe list generation. Genetic locations below p-value of 0.3 or 0.2 are selected. The statistical probes mapping to these genetic locations, with the markers from step 2, form the high value markers for EpiSwitch™ PCR translation.

### Array Design and Processing

### Array Design

1. Genetic loci are processed using the SII software (currently v3.2) to:
   a. Pull out the sequence of the genome at these specific genetic loci (gene sequence with 50 kb upstream and 20 kb downstream)
   b. Define the probability that a sequence within this region is involved in CCs
   c. Cut the sequence using a specific RE
   d. Determine which restriction fragments are likely to interact in a certain orientation
   e. Rank the likelihood of different CCs interacting together.
2. Determine array size and therefore number of probe positions available (x)
3. Pull out x/4 interactions.
4. For each interaction define sequence of 30 bp to restriction site from part 1 and 30 bp to restriction site of part 2. Check those regions that are not repeats, if so exclude and take next interaction down on the list. Join both 30 bp to define probe.
5. Create list of x/4 probes plus defined control probes and replicate 4 times to create list to be created on array
6. Upload list of probes onto Agilent Sure design website for custom CGH array.
7. Use probe group to design Agilent custom CGH array.

### Array Processing

1. Process samples using EpiSwitch™ Standard Operating Procedure (SOP) for template production.
2. Clean up with ethanol precipitation by array processing laboratory.
3. Process samples as per Agilent SureTag complete DNA labelling kit—Agilent Oligonucleotide Array-based CGH for Genomic DNA Analysis Enzymatic labelling for Blood, Cells or Tissues
4. Scan using Agilent C Scanner using Agilent feature extraction software.

EpiSwitch™ biomarker signatures demonstrate high robustness, sensitivity and specificity in the stratification of complex disease phenotypes. This technology takes advantage of the latest breakthroughs in the science of epigenetics, monitoring and evaluation of chromosome conformation signatures as a highly informative class of epigenetic biomarkers. Current research methods deployed in academic environment require from 3 to 7 days for biochemical processing of cellular material in order to detect CCSs. Those procedures have limited sensitivity, and reproducibility; and furthermore, do not have the benefit of the targeted insight provided by the EpiSwitch™ Analytical Package at the design stage.

### EpiSwitch™ Array in Silico Marker Identification

CCS sites across the genome are directly evaluated by the EpiSwitch™ Array on clinical samples from testing cohorts for identification of all relevant stratifying lead biomarkers. The EpiSwitch™ Array platform is used for marker identification due to its high-throughput capacity, and its ability to screen large numbers of loci rapidly. The array used was the Agilent custom-CGH array, which allows markers identified through the in silico software to be interrogated.

### Episwitch™ PCR

Potential markers identified by EpiSwitch™ Array are then validated either by EpiSwitch™ PCR or DNA sequencers (i.e. Roche 454, Nanopore MinION, etc.). The top PCR markers which are statistically significant and display the best reproducibility are selected for further reduction into the final EpiSwitch™ Signature Set, and validated on an independent cohort of samples. EpiSwitch™ PCR can be performed by a trained technician following a standardised operating procedure protocol established. All protocols and manufacture of reagents are performed under ISO 13485 and 9001 accreditation to ensure the quality of the work and the ability to transfer the protocols. EpiSwitch™ PCR and EpiSwitch™ Array biomarker platforms are compatible with analysis of both whole blood and cell lines. The tests are sensitive enough to detect abnormalities in very low copy numbers using small volumes of blood.

### Annotations Used in the Tables

The tables give names to particular sets of probes and primers to be used in aspects of the invention. Alternative primer names for the following PCR marker sets are shown below:

For OBD168-669.671: OBD168-013.015

For OBD168-1729.1731: OBD168-265.267

For OBD168-1841.1843: OBD168-353.355

For OBD168-1361.1363: OBD168-97.99

For OBD168-1529.1531: OBD168-645.647

For OBD168-681.683: OBD168-661.663

For OBD168-921.923: OBD168-717.719

For OBD168-893.895: OBD168-705.707

For OBD168-1273.1275: OBD168-749.751

For OBD168-1629.1631: OBD168-737.739

For OBD168-885.887: OBD168-733.735

For OBD168-1741.1743: OBD168-781.783

The invention is illustrated by the following Examples:

### Chromatin Conformation Signature Analysis in Early Vs Late Fibrosis/Scleroderma Phenotypes

In this work systemic sclerosis is being used as a model disease to investigate the chromatin conformation signature relevant to fibrosis.

Systemic sclerosis (scleroderma, SSc) is a heterogeneous fibrosis disease in which clinical outcomes vary widely. Predicting outcomes on an individual basis remains challenging despite progress made through autoantibody analysis and gene expression profiling. Effective targeted therapies are evolving and accurately predicting outcomes is important to enable patient stratification for therapy.

Scleroderma (systemic sclerosis, SSc) is a severe connective tissue disease in which an autoimmune process at onset is associated with downstream fibrosis of the skin and internal organs, as well as vascular damage. In general, SSc is difficult to treat and patients continue to suffer a disabling, progressive and potentially lethal disease. Comprehensive genetic studies have indicated association of scleroderma susceptibility with genes involved in innate and adaptive immunity. Activated macrophages are seen to infiltrate into the perivascular tissue at an early stage of the disease, expressing markers of the profibrotic M2 phenotype, such as CD206 and secreting growth factors such as TGFβ which drive fibrosis. Macrophages derived from SSc patients display an activation signature characterised by high CD206 and elevated arginase1, highest in patients with severe disease. Soluble CD206 is elevated in plasma and tissue fluid of patients as a biomarker of the M2 activation state. In co-culture, SSc macrophages secrete factors which stimulate fibroblasts to increase collagen I production.

In the present work, through profiling of blood derived macrophages a mixed activation state of macrophages has been identified in this disease. This includes identifying a macrophage activation signature which indicates a combined pro-inflammatory as well as M2-like pro-fibrotic state. Macrophages form patients release inflammatory proteins characteristic of this disease including IL-6, but are also pro-fibrotic, stimulating fibroblasts in co-culture. Using a signature across 3 markers (IFN¥, CD206 and Arg1) an activation signature present in 50% of patients has been identified, which is not seen in healthy control samples. These characteristics persist in tissue culturing implying that the signature results from an epigenetic change which fundamentally alters the macrophage state. Likely initiating factors including Th2 cytokines encountered in the disease microenvironment, tissue stiffness in the disease microenvironment, or stimulation by antibody/antigen complexes carried into the cells systemically.

Chromatin Conformation Signature (CCS) profiling of peripheral blood for systemic epigenetic deregulations was used in this work. The EpiSwitch platform offering high throughput and resolution chromosome conformation (3C) capture detects significant regulatory changes in 3D genome architecture and maps long range interaction between distant genomic locations. This, in turn, reveals the spatial disposition and physical properties of the chromosome, such as chromatin loops and inter-chromosomal connections, which have a role in network organization and genetic epistasis controlling gene expression.

This methodology was applied to patients with SSc to identify CCS associated with different phenotypes and can be used to stratify and identify patients into pathogenic subtypes.

We aimed to determine significant chromatin conformation signatures associated with presence of scleroderma and also detection of early and late phenotypes of scleroderma.

The EpiSwitch—based chromosome conformation capture (3C) method was applied to blood samples from early phenotype, and late phenotype SSc patients. Intact nuclei were isolated from peripheral blood mononuclear cells and subjected to formaldehyde fixation resulting in crosslinking between physically touching segments of the genome via contacts between their DNA bound proteins. For quantification of cross-linking frequencies, the cross-linked DNA was digested and then subjected to ligation. Cross-linking was then reversed and individual ligation products detected and quantified by EpiSwitch custom oligo array annotated across the whole genome to the anchoring sites of 3D genome architecture.

The UK sample cohort consisted of 4 Healthy Controls and 6 Scleroderma patient samples. The 6 Scleroderma patients were split between Early and Late stage disease. EpiSwitch libraries were generated from the received sample cohort (n=8) using the Robotic Delta9.1 with Qiagen column purification protocols. A pooled control was created from the 4 extracted Healthy control samples. These libraries were processed for using on the custom designed Agilent CGH array.

### Results

7 significant CCSs were found over the HLA-C, HLA-B and TNF regions on Chromosome 6 in the early phenotype. The top 10 pathways for genetic locations associated to the CCSs are shown in the table below for the early phenotype.

| | GeneSet |
|---|---|
| 1 | Natural Killer cell mediated cytotoxicity |
| 2 | Immunoregulatory interactions between a |
| | lymphoid cell and a non-lymphoid cell |
| 3 | Antigen Processing & presentation |
| 4 | Phagosome |
| 5 | Graft versus host disease |
| 6 | Type 1 diabetes mellitus |
| 7 | Osteoclast differentiation |
| 8 | Class 1 MHC mediated antigen processing & presentation |
| 9 | Rheumatoid Arthritis |
| 10 | GnRH signalling pathway |

2 significant CCSs were found centred around the IFNG region of chromosome 12 in the late phenotype. The top 10 pathways for genetic locations associated to significant CCSs are shown in the table below for the late phenotype.

| | GeneSet |
|---|---|
| 1 | Surfactant metabolism |
| 2 | IL12 signalling mediated by STAT4 |
| 3 | Protein digestion and absorption |
| 4 | Calcineruin regulated NFAT dependent transcription in lymphocytes |
| 5 | Transcriptional mis-regulation in cancer |
| 6 | Kaposi's sarcoma associated herpes virus infection |
| 7 | IL2 mediated signalling events |
| 8 | Inflammatory bowel disease |
| 9 | Interleukin-20 Family signalling |
| 10 | AP-1 transcription factor network |

Table 1 and Table 2 show the markers that were identified by this work. They represent part of the 3D genomic regulatory control. There were distinct CCSs in the early phenotype compared to the late showing the CCSs change as the disease progresses and varies between phenotypes. The CCSs can be linked to each clinically defined subgroup to be used as a biomarker tool to predict outcome and progression in patients. The present work therefore provides both diagnostic and prognostic markers.

Subsequent work done in the same way identified the markers shown in Tables 3 to 6. The data provided in Tables 1 to 6 show the efficacy of these markers in identifying the subgroups they are associated with.

It must be appreciated that identifying the markers shown in the tables required the testing of many potential markers. A total of 170229 markers were studied in an array. In the first analysis, they were shortlisted to 100 diagnostic (HC VS SSc) and prognostic (late SSc VS early SSc) markers, (170029 dropped out). In the second analysis, a shortlist of top 200 prognostic (unique markers present in early and late SSc) and 200 diagnostic (unique markers present in SSc and HC) markers prepared (169829 dropped out).

For the second set of markers of Tables 3 to 6 additional modelling values has been provided based on a SHAPLEY analysis. The markers were used to build a Random Forest model and the markers contribution to the models was calculated using SHAPLEY. The results are shown in Tables 7 to 10, where the SHAPLEY difference column in each tab is the variable to rank importance, the larger the difference the more important it is in the model.

The top 100 markers for either early v late disease or SSc v pHC (healthy control), generate an average model accuracy of 94%, this is compared to a mean average of 45% when you randomly select 100 markers and do this 100 times. This difference in classifying accuracy clearly demonstrates the power of the statistically selected markers.

In conclusion, the present work has led to the identification of markers which each have a powerful association with a distinct characteristic of fibrosis, and therefore individual markers or small numbers of these markers can be the basis of an effective test for the stage or presence of fibrosis.

TABLE 1.a1

| | probe | gene.index | RP/Rsum | FC: (class1/class2) |
|---|---|---|---|---|
| 1 | ORF1_chr9_117652733_117662476_117782539_117784298_FF_1 | 27640 | 66.27 | 0.1915 |
| 2 | ORF1_chr21_21135567_21136687_21190498_21197349_RR_1 | 17045 | 79.99 | 0.3925 |
| 3 | ORF1_chr12_108190915_108198003_108364371_108366987_FR_1 | 41612 | 75.78 | 0.3598 |
| 4 | ORF1_chr8_138185388_138192430_138316302_138317782_FF_1 | 101290 | 88.9 | 0.3921 |
| 5 | ORF1_chr6_104807984_104813896_104858263_104859297_FR_1 | 123015 | 60.98 | 0.3632 |
| 6 | ORF1_chr7_73838719_73840112_73998797_74003463_FF_1 | 88433 | 52.65 | 0.3224 |
| 7 | ORF1_chr6_1490191_1492799_1612178_1614243_FR_1 | 109136 | 40.25 | 0.2393 |
| 8 | ORF1_chr13_109624423_109630621_109869387_109876529_RF_1 | 116786 | 103.6 | 0.4592 |
| 9 | ORF1_chr3_171788979_171791691_171971435_171976171_RF_1 | 38725 | 162 | 0.2839 |
| 10 | ORF1_chr13_102842718_102847079_102927740_102931657_FR_1 | 65746 | 168.2 | 0.4991 |
| 11 | ORF1_chrX_40971005_40973842_41006985_41010257_RF_1 | 93949 | 159.5 | 0.3908 |
| 12 | ORF1_chr4_26084879_26086604_26290850_26293788_FR_1 | 33330 | 173.8 | 0.476 |
| 13 | ORF1_chr13_109869387_109876529_109901846_109907608_FF_1 | 60814 | 196.5 | 0.5216 |
| 14 | ORF1_chr11_21505657_21511320_21625659_21628201_FR_1 | 69339 | 180.6 | 0.5138 |
| 15 | ORF1_chr13_102842718_102847079_102867709_102871733_FF_1 | 62134 | 157.6 | 0.4919 |
| 16 | ORF1_chr13_102842718_102847079_102952727_102953846_FF_1 | 57139 | 194.4 | 0.5129 |
| 17 | ORF1_chr3_179490429_179499685_179696553_179701788_RF_1 | 32920 | 153.2 | 0.1938 |
| 18 | ORF1_chr13_109869387_109876529_109994965_109996722_FR_1 | 130780 | 191.1 | 0.5202 |
| 19 | ORF1_chr13_42949707_42958335_43086110_43092272_RF_1 | 34008 | 188.2 | 0.1628 |
| 20 | ORF1_chr10_93375413_93378505_93410956_93413925_FR_1 | 97132 | 137.7 | 0.4814 |
| 21 | ORF1_chr1_182254591_182258498_182271295_182272617_RR_1 | 106543 | 144.6 | 0.4902 |
| 22 | ORF1_chr6_104807984_104813896_104859297_104866639_FF_1 | 135912 | 150.5 | 0.4555 |
| 23 | ORF1_chr3_35637910_35639568_35705745_35720206_RF_1 | 28231 | 206.9 | 0.524 |
| 24 | ORF1_chr8_81130626_81134961_81156168_81161109_RF_1 | 33879 | 135.9 | 0.3518 |
| 25 | ORF1_chr11_61243450_61246026_61388261_61391790_FR_1 | 117098 | 242 | 0.5319 |
| 26 | ORF1_chr4_107675768_107680919_107825397_107833109_FF_1 | 63807 | 246.5 | 0.5419 |
| 27 | ORF1_chr1_193456875_193464580_193519461_193528788_RR_1 | 11362 | 262.4 | 0.501 |
| 28 | ORF1_chr11_79134716_79152601_79219225_79225698_FR_1 | 33877 | 277.3 | 0.1144 |
| 29 | ORF1_chr6_15282103_15283199_15362212_15366665_RR_1 | 33469 | 294.6 | 0.2127 |
| 30 | ORF1_chr11_35101113_35109487_35250591_35252942_RR_1 | 70719 | 292.6 | 0.5615 |
| 31 | ORF1_chr11_111144097_111148048_111222819_111226135_FF_1 | 26876 | 289 | 0.5585 |
| 32 | ORF1_chr18_62522952_62526928_62633916_62637110_RR_1 | 73540 | 305.3 | 0.5596 |
| 33 | ORF1_chr18_63796182_63806119_63902371_63912126_RF_1 | 46660 | 312.7 | 0.5488 |
| 34 | ORF1_chr4_59658576_59668116_59701046_59706041_RF_1 | 34142 | 320.9 | 0.4449 |
| 35 | ORF1_chr2_68857299_68862999_68948041_68951799_RF_1 | 26296 | 348.7 | 0.482 |
| 36 | ORF1_chr14_61751727_61758003_61810071_61812335_FR_1 | 139473 | 344.4 | 0.5737 |
| 37 | ORF1_chr21_33072222_33074551_33276086_33283169_RF_1 | 38327 | 398.6 | 0.4507 |
| 38 | ORF1_chr11_111222819_111226135_111261363_111265949_FR_1 | 43619 | 383.4 | 0.5574 |
| 39 | ORF1_chr4_38671979_38673590_38888275_38891480_RF_1 | 36577 | 389.3 | 0.2779 |
| 40 | ORF1_chr3_114010550_114016075_114047949_114051357_RF_1 | 33335 | 405.1 | 0.2532 |
| 41 | ORF1_chr14_61490070_61495640_61751727_61758003_RF_1 | 53496 | 398.1 | 0.5138 |
| 42 | ORF1_chr15_67887204_67889122_68052888_68053975_FR_1 | 38981 | 434.3 | 0.2186 |
| 43 | ORF1_chr14_96740036_96741996_96820569_96823739_RF_1 | 129511 | 447.6 | 0.5956 |
| 44 | ORF1_chr8_116637960_116642220_116837668_116843681_FR_1 | 87182 | 461.1 | 0.3976 |
| 45 | ORF1_chr7_43764036_43765883_43792676_43795674_RR_l | 37245 | 487.1 | 0.3162 |
| 46 | ORF1_chr11_78798294_78806874_79036088_79041510_RF_1 | 40619 | 486.4 | 0.6055 |
| 47 | ORF1_chr12_103575776_103578331_103663674_103664970_FR_1 | 32922 | 505.5 | 0.2152 |
| 48 | ORF1_chr6_13375878_13378557_13523152_13526281_FF_1 | 33742 | 556.4 | 0.2342 |
| 49 | ORF1_chr11_21505657_21511320_21751405_21757053_FR_1 | 131989 | 552.9 | 0.4888 |
| 50 | ORF1_chr5_54531220_54532270_54669234_54674643_FF_1 | 22667 | 574.6 | 0.4734 |

TABLE 1.a2

| | | | | Probe sequence |
|---|---|---|---|---|
| | pfp | P.value | CCSs | 60 mer |
| 1 | 0.06903 | 0.000001957 | Late | ACAAAACTGATGATGCCTTGCAACAAACTCGATAGAAGAGAAAAACAAGCTCAGAGCAAC |
| 2 | 0.0699 | 0.000002972 | Late | CCAACGGCATATAAAACTGAGAAGAGCTTCGACTCTGAGTCTTCTTCTTCAGTGTTCTGG |
| 3 | 0.07439 | 0.000002635 | Late | TCCTTTCCTTCAAGTCTTTAATCAAACCTCGATGCAGAACCTGAATGGACAACTTCTGGA |
| 4 | 0.0757 | 0.000003755 | Late | TGTAAGACTATCAAATTAGCCAAAATCTTCGAAAGCAGAAAATAAAAAGATAGATTTGGT |
| 5 | 0.07646 | 0.000001625 | Late | ACAAAAATTAGAAAAGTAACTGATAACCTCGACTACGAAAAATAAGTAAGAGTAGCACTT |
| 6 | 0.08262 | 0.000001171 | Late | ACTGAGGGCACCGACTATCAGCACCGCGTCGAAAATCATAATCTGGGGCCCTCTCTCCTC |
| 7 | 0.09039 | 6.404E−07 | Late | ATATCCAATCAGAGTGGAAATATCAGAGTCGAAACTAAAAAAAAAAAATCCAATTAAAAA |
| 8 | 0.09278 | 0.000005259 | Late | GGGTAGGGAAGTAAAAGAAAATGGTGCTTCGATTCCCATTGAAGCTAAGTCTGCAGTTTA |
| 9 | 0.1239 | 0.00001404 | Late | TTCAGTTCAATGAGGAAAAGTCTCATAATCGAGGATGAATAAAACAGACATTGTCCTCAA |
| 10 | 0.1267 | 0.00001526 | Late | GAGGTCAGTAAGGAGAGCAGAGTAGACTTCGATTCTCCTCTTATCACTCCCTTCCTTGAA |
| 11 | 0.1278 | 0.00001358 | Late | ATGCGGTGCTGAGTTGTGCTATGGCAACTCGAACTCCTAACCTCAAATGGTCTTCCCGCC |
| 12 | 0.1284 | 0.00001638 | Late | AGCCATCAAACAGAACCGGAAAAGTGGCTCGAGAAGAGAATCGTAAATGAAATACATAAA |
| 13 | 0.1314 | 0.00002142 | Late | GGGTAGGGAAGTAAAAGAAAATGGTGCTTCGATCCTCTCCATCAAGTCTCCTCTCTCTAG |
| 14 | 0.1323 | 0.00001781 | Late | CCTACAACTTTCTTATTTGTACTTTTCCTCGATAAATTTATGTTTATTTAAAAAAATTTG |
| 15 | 0.1333 | 0.00001322 | Late | GAGGTCAGTAAGGAGAGCAGAGTAGACTTCGAAGAATATTTAATAAAATGTAAATATGCC |
| 16 | 0.1343 | 0.00002094 | Late | GAGGTCAGTAAGGAGAGCAGAGTAGACTTCGAATTTCTTTATTGACATGTTCTCACTCAT |
| 17 | 0.135 | 0.00001243 | Late | CCTTCTCTTTTCTLCCTTTTCCTTTCTTTCGATAGATAGAGACATATATGAGAGGGGATT |
| 18 | 0.1354 | 0.00002015 | Late | GGGTAGGGAAGTAAAAGAAAATGGTGCTTCGAGTTAAGACTGTTCTGAGTCCTGTAAAAA |
| 19 | 0.1376 | 0.0000195 | Late | ACTGCTTTTTGCCATAAAGCCTACCTTATCGAGAAAATTCTTAAAAGAACACTTTTTCCT |
| 20 | 0.1389 | 0.00000984 | Late | ACATAAAATGATATTTCAGATTTTGCCGTCGAGCTATGTGAGTCTGAGTAAGTTTCTTAA |
| 21 | 0.1405 | 0.00001095 | Late | GCGGAGATCTTTCAAGAAATTTATTTAATCGATAGTATGTACCCTTGATATGATATGATG |
| 22 | 0.1406 | 0.00001195 | Late | ACAAAAATTAGAAAAGTAACTGATAACCTCGAGTCATCTATTTGATATCTCCACTTCAGT |
| 23 | 0.1409 | 0.00002397 | Late | CACTGAGTATTTTCATCTAATCTTTCACTCGAATAGTTGGAAAATAATTACCTACTCATC |
| 24 | 0.1499 | 0.000009559 | Late | ATGGAAATCTGAGTAAAGAAATTCATGGTCGAGCCAAGAGGAAGGAAGAAACGGCGGTGG |
| 25 | 0.1903 | 0.0000337 | Late | CCTGGCTCCTTCCTGTATTATGCTCCCTTCGAGAAGTAGAATATCTGAATGTTCTTATAT |
| 26 | 0.1904 | 0.00003508 | Late | AAATACAAAGAAAAACATCAGAGCAGTATCGACCATGCACTCAGAAGCTTTTACAGGGAA |
| 27 | 0.2102 | 0.00004021 | Late | CACCTCATCAGCACTCAGAAGAGAACAGTCGACAGTTAAGAGATCCTGGTTAAGGGATGG |
| 28 | 0.2284 | 0.00004531 | Late | TACAACAATTTATTTTTGGTTTAAAAGCTCGAGCTCAGCTTTCCAGCATGTGTAAGCTGT |
| 29 | 0.2352 | 0.00005166 | Late | TGGTCTCTACTTAAAATACAGAAATTGGTCGAAAATATATCAGTTTAAGTGTTATTGAGG |
| 30 | 0.2395 | 0.00005091 | Late | ACATACCCCTAAGAATGCTCAGAAATACTCGAGAAATGATATGTTTGTATACTATCCTTT |
| 31 | 0.2413 | 0.00004958 | Late | CTGTTCTAAGCCTAAAAAGAATATATATTCGACTACTGTGAATAGGGCTGCAATAAGCAT |
| 32 | 0.2462 | 0.00005582 | Late | CCTCACCTCCGCCAATCAGCACCGCGAATCGATCCCTTTGGATTCTCAGGTCCCCATCTT |
| 33 | 0.2515 | 0.00005881 | Late | ATATAGAAATGGTACTGATTTTTGTACGTCGAAATTAAAAGTTCAAAAATACATTTCTTA |
| 34 | 0.2583 | 0.00006222 | Late | GGGGAAGATAAGTGATCATATTTCTACTTCGATCAAGCATACATAATAGACATCTACAGA |
| 35 | 0.2919 | 0.00007446 | Late | AGTCTAAAATAGTGGTTTGAACAAGTAGTCGAGGAGGAAAGAAAAACCTCTCAGGTCCAT |
| 36 | 0.2922 | 0.00007247 | Late | ATGCAAGAGAAATTACATAACAGAGGGCTCGATGTTTACCTTGAACTGAGTAAAATCAAA |
| 37 | 0.3459 | 0.00009803 | Late | AGCCATGGACAACTTATTGTGTTCAAGTTCGATTTGTTACAGACGTTAACAAATTCCTTT |
| 38 | 0.3469 | 0.00009095 | Late | ATGCTTATTGCAGCCCTATTCACAGTAGTCGAATTATTTTCAAATAACATCATAAAATTC |
| 39 | 0.3479 | 0.00009367 | Late | TCCCTGTCTTCTAAATAATAAGAGAGAATCGACACTAGACTTGTTTAGAAGACCTCACTG |
| 40 | 0.348 | 0.0001011 | Late | GATATGCAGCTACTATCTTACTGCATCCTCGATATTAAATCCCTACTAACTTCCAATGAT |
| 41 | 0.3539 | 0.00009779 | Late | ATGCAAGAGAAATTACATAACAGAGGGCTCGACTCATGAGTCTGACTGCTTTGTGCCTTA |
| 42 | 0.3878 | 0.0001154 | Late | AGCTGACTTATGTACCAGATACTTTGTTTCGATCAGGGTAGACTGAGAAGTGAATGTGAA |
| 43 | 0.401 | 0.0001222 | Late | GCAGATCTCTAGAACTTAATTTGCAAATTCGATTAAATGCTTATTATGTGTTTATTTAGC |
| 44 | 0.4147 | 0.0001293 | Late | TAACATTCTGATACCACTACACCTGAGTTCGACTAATTATGATGGTGAAGGAGCAGAAGA |
| 45 | 0.44 | 0.0001434 | Late | TCTAGAGAGAACATCTTTCTTCAGCAGATCGAAGTGTTCTTTCTCTGTATTTGTCTCGTT |
| 46 | 0.4485 | 0.000143 | Late | CATTGGAGGCACTCAAAAAAAAAAAAAATCGATTGTGACCTAATTTGATAACAGGGCTTT |
| 47 | 0.4617 | 0.0001537 | Late | AAATTATATTTTCTTAGGTTAATCGTCCTCGATCATTATATTTAAGGCCCACACTAATTC |
| 48 | 0.5196 | 0.0001841 | Late | ACCTGCTGGGACAACTGAAAGATTATTATCGAGTCTAAACATCTACATGGGATTAGTAAT |
| 49 | 0.524 | 0.0001819 | Late | CCTACAACTTTCTTATTTGTACTTTTCCTCGAGGAAATAATTCACTAGGTAAAGGGAGTA |
| 50 | 0.5305 | 0.0001954 | Late | TACAGATATGCTTGTTGTATGTCACGTGTCGACATAAATATTTGTTGAACAAGTAAATGA |

TABLE 1.a3

| | Probe Location | | | | | 4 kb Sequence Location | | | | |
|---|---|---|---|---|---|---|---|---|---|---|
| | Chr | Start1 | End1 | Start2 | End2 | Chr | Start1 | End1 | Start2 | End2 |
| 1 | chr9 | 117662445 | 117662474 | 117784267 | 117784296 | chr9 | 117658475 | 117662474 | 117780297 | 117784296 |
| 2 | chr21 | 21135569 | 21135598 | 21190500 | 21190529 | chr21 | 21135569 | 21139568 | 21190500 | 21194499 |
| 3 | chr12 | 108197972 | 108198001 | 108364373 | 108364402 | chr12 | 108194002 | 108198001 | 108364373 | 108368372 |
| 4 | chr8 | 138192399 | 138192428 | 138317751 | 138317780 | chr8 | 138188429 | 138192428 | 138313781 | 138317780 |
| 5 | chr6 | 104813865 | 104813894 | 104858265 | 104858294 | chr6 | 104809895 | 104813894 | 104858265 | 104862264 |
| 6 | chr7 | 73840081 | 73840110 | 74003432 | 74003461 | chr7 | 73836111 | 73840110 | 73999462 | 74003461 |
| 7 | chr6 | 1492768 | 1492797 | 1612180 | 1612209 | chr6 | 1488798 | 1492797 | 1612180 | 1616179 |
| 8 | chr13 | 109624425 | 109624454 | 109876498 | 109876527 | chr13 | 109624425 | 109628424 | 109872528 | 109876527 |
| 9 | chr3 | 171788981 | 171789010 | 171976140 | 171976169 | chr3 | 171788981 | 171792980 | 171972170 | 171976169 |
| 10 | chr13 | 102847048 | 102847077 | 102927742 | 102927771 | chr13 | 102843078 | 102847077 | 102927742 | 102931741 |
| 11 | chrX | 40971007 | 40971036 | 41010226 | 41010255 | chrX | 40971007 | 40975006 | 41006256 | 41010255 |
| 12 | chr4 | 26086573 | 26086602 | 26290852 | 26290881 | chr4 | 26082603 | 26086602 | 26290852 | 26294851 |
| 13 | chr13 | 109876498 | 109876527 | 109907577 | 109907606 | chr13 | 109872528 | 109876527 | 109903607 | 109907606 |
| 14 | chr11 | 21511289 | 21511318 | 21625661 | 21625690 | chr11 | 21507319 | 21511318 | 21625661 | 21629660 |
| 15 | chr13 | 102847048 | 102847077 | 102871702 | 102871731 | chr13 | 102843078 | 102847077 | 102867732 | 102871731 |
| 16 | chr13 | 102847048 | 102847077 | 102953815 | 102953844 | chr13 | 102843078 | 102847077 | 102949845 | 102953844 |
| 17 | chr3 | 179490431 | 179490460 | 179701757 | 179701786 | chr3 | 179490431 | 179494430 | 179697787 | 179701786 |
| 18 | chr13 | 109876498 | 109876527 | 109994967 | 109994996 | chr13 | 109872528 | 109876527 | 109994967 | 109998966 |
| 19 | chr13 | 42949709 | 42949738 | 43092241 | 43092270 | chr13 | 42949709 | 42953708 | 43088271 | 43092270 |
| 20 | chr10 | 93378474 | 93378503 | 93410958 | 93410987 | chr10 | 93374504 | 93378503 | 93410958 | 93414957 |
| 21 | chr1 | 182254593 | 182254622 | 182271297 | 182271326 | chr1 | 182254593 | 182258592 | 182271297 | 182275296 |
| 22 | chr6 | 104813865 | 104813894 | 104866608 | 104866637 | chr6 | 104809895 | 104813894 | 104862638 | 104866637 |
| 23 | chr3 | 35637912 | 35637941 | 35720175 | 35720204 | chr3 | 35637912 | 35641911 | 35716205 | 35720204 |
| 24 | chr8 | 81130628 | 81130657 | 81161078 | 81161107 | chr8 | 81130628 | 81134627 | 81157108 | 81161107 |
| 25 | chr11 | 61245995 | 61246024 | 61388263 | 61388292 | chr11 | 61242025 | 61246024 | 61388263 | 61392262 |
| 26 | chr4 | 107680888 | 107680917 | 107833078 | 107833107 | chr4 | 107676918 | 107680917 | 107829108 | 107833107 |
| 27 | chr1 | 193456877 | 193456906 | 193519463 | 193519492 | chr1 | 193456877 | 193460876 | 193519463 | 193523462 |
| 28 | chr11 | 79152570 | 79152599 | 79219227 | 79219256 | chr11 | 79148600 | 79152599 | 79219227 | 79223226 |
| 29 | chr6 | 15282105 | 15282134 | 15362214 | 15362243 | chr6 | 15282105 | 15286104 | 15362214 | 15366213 |
| 30 | chr11 | 35101115 | 35101144 | 35250593 | 35250622 | chr11 | 35101115 | 35105114 | 35250593 | 35254592 |
| 31 | chr11 | 111148017 | 111148046 | 111226104 | 111226133 | chr11 | 111144047 | 111148046 | 111222134 | 111226133 |
| 32 | chr18 | 62522954 | 62522983 | 62633918 | 62633947 | chr18 | 62522954 | 62526953 | 62633918 | 62637917 |
| 33 | chr18 | 63796184 | 63796213 | 63912095 | 63912124 | chr18 | 63796184 | 63800183 | 63908125 | 63912124 |
| 34 | chr4 | 59658578 | 59658607 | 59706010 | 59706039 | chr4 | 59658578 | 59662577 | 59702040 | 59706039 |
| 35 | chr2 | 68857301 | 68857330 | 68951768 | 68951797 | chr2 | 68857301 | 68861300 | 68947798 | 68951797 |
| 36 | chr14 | 61757972 | 61758001 | 61810073 | 61810102 | chr14 | 61754002 | 61758001 | 61810073 | 61814072 |
| 37 | chr21 | 33072224 | 33072253 | 33283138 | 33283167 | chr21 | 33072224 | 33076223 | 33279168 | 33283167 |
| 38 | chr11 | 111226104 | 111226133 | 111261365 | 111261394 | chr11 | 111222134 | 111226133 | 111261365 | 111265364 |
| 39 | chr4 | 38671981 | 38672010 | 38891449 | 38891478 | chr4 | 38671981 | 38675980 | 38887479 | 38891478 |
| 40 | chr3 | 114010552 | 114010581 | 114051326 | 114051355 | chr3 | 114010552 | 114014551 | 114047356 | 114051355 |
| 41 | chr14 | 61490072 | 61490101 | 61757972 | 61758001 | chr14 | 61490072 | 61494071 | 61754002 | 61758001 |
| 42 | chr15 | 67889091 | 67889120 | 68052890 | 68052919 | chr15 | 67885121 | 67889120 | 68052890 | 68056889 |
| 43 | chr14 | 96740038 | 96740067 | 96823708 | 96823737 | chr14 | 96740038 | 96744037 | 96819738 | 96823737 |
| 44 | chr8 | 116642189 | 116642218 | 116837670 | 116837699 | chr8 | 116638219 | 116642218 | 116837670 | 116841669 |
| 45 | chr7 | 43764038 | 43764067 | 43792678 | 43792707 | chr7 | 43764038 | 43768037 | 43792678 | 43796677 |
| 46 | chr11 | 78798296 | 78798325 | 79041479 | 79041508 | chr11 | 78798296 | 78802295 | 79037509 | 79041508 |
| 47 | chr12 | 103578300 | 103578329 | 103663676 | 103663705 | chr12 | 103574330 | 103578329 | 103663676 | 103667675 |
| 48 | chr6 | 13378526 | 13378555 | 13526250 | 13526279 | chr6 | 13374556 | 13378555 | 13522280 | 13526279 |
| 49 | chr11 | 21511289 | 21511318 | 21751407 | 21751436 | chr11 | 21507319 | 21511318 | 21751407 | 21755406 |
| 50 | chr5 | 54532239 | 54532268 | 54674612 | 54674641 | chr5 | 54528269 | 54532268 | 54670642 | 54674641 |

TABLE 1.a4

| | probe | Primer ID |
|---|---|---|
| 1 | ORF1_9_117652733_117662476_117782539_117784298_FF | OBD168-001 |
| 2 | ORF1_21_21135567_21136687_21190498_21197349_RR | OBD168-005 |
| 3 | ORF1_12_108190915_108198003_108364371_108366987_FR | OBD168-009 |
| 4 | ORF1_8_138185388_138192430_138316302_138317782_FF | OBD168-013 |
| 5 | ORF1_6_104807984_104813896_104858263_104859297_FR | OBD168-017 |
| 6 | ORF1_7_73838719_73840112_73998797_74003463_FF | OBD168-021 |
| 7 | ORF1_6_1490191_1492799_1612178_1614243_FR | OBD168-025 |
| 8 | ORF1_13_109624423_109630621_109869387_109876529_RF | OBD168-029 |
| 9 | ORF1_3_171788979_171791691_171971435_171976171_RF | OBD168-033 |
| 10 | ORF1_13_102842718_102847079_102927740_102931657_FR | OBD168-037 |
| 11 | ORF1_X_40971005_40973842_41006985_41010257_RF | OBD168-041 |
| 12 | ORF1_4_26084879_26086604_26290850_26293788_FR | OBD168-045 |
| 13 | ORF1_13_109869387_109876529_109901846_109907608_FF | OBD168-049 |
| 14 | ORF1_11_21505657_21511320_21625659_21628201_FR | OBD168-053 |
| 15 | ORF1_13_102842718_102847079_102867709_102871733_FF | OBD168-057 |
| 16 | ORF1_13_102842718_102847079_102952727_102953846_FF | OBD168-061 |
| 17 | ORF1_3_179490429_179499685_179696553_179701788_RF | OBD168-065 |
| 18 | ORF1_13_109869387_109876529_109994965_109996722_FR | OBD168-069 |
| 19 | ORF1_13_42949707_42958335_43086110_43092272_RF | OBD168-073 |
| 20 | ORF1_10_93375413_93378505_93410956_93413925_FR | OBD168-077 |
| 21 | ORF1_1_182254591_182258498_182271295_182272617_RR | OBD168-081 |
| 22 | ORF1_6_104807984_104813896_104859297_104866639_FF | OBD168-085 |
| 23 | ORF1_3_35637910_35639568_35705745_35720206_RF | OBD168-089 |
| 24 | ORF1_8_81130626_81134961_81156168_81161109_RF | OBD168-093 |
| 25 | ORF1_11_61243450_61246026_61388261_61391790_FR | OBD168-097 |
| 26 | ORF1_4_107675768_107680919_107825397_107833109_FF | OBD168-101 |
| 27 | ORF1_1_193456875_193464580_193519461_193528788_RR | OBD168-105 |
| 28 | ORF1_11_79134716_79152601_79219225_79225698_FR | OBD168-109 |
| 29 | ORF1_6_15282103_15283199_15362212_15366665_RR | OBD168-113 |
| 30 | ORF1_11_35101113_35109487_35250591_35252942_RR | OBD168-117 |
| 31 | ORF1_11_111144097_111148048_111222819_111226135_FF | OBD168-121 |
| 32 | ORF1_18_62522952_62526928_62633916_62637110_RR | OBD168-125 |
| 33 | ORF1_18_63796182_63806119_63902371_63912126_RF | OBD168-129 |
| 34 | ORF1_4_59658576_59668116_59701046_59706041_RF | OBD168-133 |
| 35 | ORF1_2_68857299_68862999_68948041_68951799_RF | OBD168-137 |
| 36 | ORF1_14_61751727_61758003_61810071_61812335_FR | OBD168-141 |
| 37 | ORF1_21_33072222_33074551_33276086_33283169_RF | OBD168-145 |
| 38 | ORF1_11_111222819_111226135_111261363_111265949_FR | OBD168-149 |
| 39 | ORF1_4_38671979_38673590_38888275_38891480_RF | OBD168-153 |
| 40 | ORF1_3_114010550_114016075_114047949_114051357_RF | OBD168-157 |
| 41 | ORF1_14_61490070_61495640_61751727_61758003_RF | OBD168-161 |
| 42 | ORF1_15_67887204_67889122_68052888_68053975_FR | OBD168-165 |
| 43 | ORF1_14_96740036_96741996_96820569_96823739_RF | OBD168-169 |
| 44 | ORF1_8_116637960_116642220_116837668_116843681_FR | OBD168-173 |
| 45 | ORF1_7_43764036_43765883_43792676_43795674_RR | OBD168-177 |
| 46 | ORF1_11_78798294_78806874_79036088_79041510_RF | OBD168-181 |
| 47 | ORF1_12_103575776_103578331_103663674_103664970_FR | OBD168-185 |
| 48 | ORF1_6_13375878_13378557_13523152_13526281_FF | OBD168-189 |
| 49 | ORF1_11_21505657_21511320_21751405_21757053_FR | OBD168-193 |
| 50 | ORF1_5_54531220_54532270_54669234_54674643_FF | OBD168-197 |

TABLE 1.a5

| | Primer Sequence | Primer ID | Primer Sequence |
|---|---|---|---|
| 1 | CATCTGACCTCCTCCAGCCTCTG | OBD168-003 | CTCTGAAGCCACACCCACACTTG |
| 2 | CTTGGAGTCCCAGTGGAAAAGGTTGA | OBD168-007 | CCAAGTGTATTGTTAGCACCAGCGGT |
| 3 | ATGCTCTGCCCCACTGTCCTTGC | OBD168-011 | GTGATTTCAGTGGGTCTCCAGGG |
| 4 | CCATACTACCTTTAGGAGGAACTTGC | OBD168-015 | TGGAGAAAACGCAAACGCCTACAGGG |
| 5 | CAGTAAAGGGAACCAAAGAAAACAAC | OBD168-019 | CAACTTCCACCTTTGAATCCATTTCT |
| 6 | GGCTGCTCCTCACCTGGCTTGGT | OBD168-023 | TCCCAACCCCTTCCAGAATCTTC |
| 7 | GCAATGCTAACAGCCTTGACAGC | OBD168-027 | TTTTCTTCGTGCTGGTGCTGGTGAGC |
| 8 | CCTGGAAGGATGTCAGATGAGTCT | OBD168-031 | AAGCAATGATAGTTGGCGATACAT |
| 9 | GGGTGAGGTAGAAGTATGAAGGAAAG | OBD168-035 | CCTTCCTCCCTTGTCTGCCTGTAGAT |
| 10 | CACAGGATTGGAATGAGGAATAAAGA | OBD168-039 | CCAATGAGGGTTCCAGGGTTCAA |
| 11 | TCCTTCCTACAACACCCTGCCAG | OBD168-043 | AGTGGCAGGTAACGGCTGGGCAT |
| 12 | TGTTGAATGAATGAGCAGGAGCCAGC | OBD168-047 | CACAGCCAGTAAACAGCATTCAAGGG |
| 13 | GGCTCTGCTGGGACTTGGGACTT | OBD168-051 | AGAGTGGCTTGACCAGAGTGTGG |
| 14 | GAAGAGTGCTTGGCAGAGTGTGTGCT | OBD168-055 | TTGTTCCTGGTTTACAGCCCCTACTA |
| 15 | CTACACACAGGATTGGAATGAGGAAT | OBD168-059 | GCACACAGCGAAATAATAGTTGTTAG |
| 16 | CACAGGATTGGAATGAGGAATAAAGA | OBD168-063 | CCACAGTGTTTCCATCAGCCTGG |
| 17 | CTCCACTGCTGAAACAATAGCGACGC | OBD168-067 | CATAAGCCAACTCCCCTAATGAATCC |
| 18 | AGATGAGTCTGCCCATTGCTCCTGAG | OBD168-071 | CTCAGAAGTCAGTTCATCCATTTCCT |
| 19 | CCTCCTACAGTGATTGTGGATTTGTC | OBD168-075 | GGGTAGGAATGTGAGTGAAGCAGGAC |
| 20 | CATCCTGTCCAGATTAGGCAGCC | OBD168-079 | CCAGATACTGTGCTAAAGGCTTCGCC |
| 21 | CAGGGAGAAGGAGAAGGAGATTTC | OBD168-083 | TGACCTTGACAGTTTTGAGGAGTA |
| 22 | GCTTCCAGCCAACATTTGAGTGCCTC | OBD168-087 | CCAGGTATGGAAAAGGAGATTGTGTC |
| 23 | CCTGGTCATTGGTGAAGGTCTGGCAT | OBD168-091 | GACACCCAACCATAATGACAGAGATT |
| 24 | GGATGTGTGAGAGAAAGGGCATTGGC | OBD168-095 | TTTACACTTTCGCCTTCTCTTCCAGC |
| 25 | TGGAGGCATCTCTGACACCAATCA | OBD168-099 | GAAAGAATACACTGATGAAGCCAT |
| 26 | ACCTCCTGGGATGTCTCAGTGGC | OBD168-103 | GAGGCAGATGTGGGAGGTTTGCT |
| 27 | GCTGGGTATGGCTGGTTTGTGTTTTA | OBD168-107 | CCTGCTTTCCATAAACCTCTGCTTCT |
| 28 | AGAGGTTCATCGTCTCCAGGTCATAG | OBD168-111 | TTGTTGGGTGTGATGTCCTCCACTTC |
| 29 | GACCAGGAGTTCAAGACCAGCCT | OBD168-115 | ATGTAACCCTGAGAGAAGACCTC |
| 30 | GGCAAGGGCTTATTTAGGAGCAT | OBD168-119 | GATTCACAGGAAGAAAACTGGAACTA |
| 31 | GCATAGATGGGTTAGTCAGAGACCAC | OBD168-123 | GGTCTCACAATCACACTGCTGGGAAT |
| 32 | TGGGTGAGGAGGTGGAGCAGCCG | OBD168-127 | AAATACGCCAGGACACAGGCAGC |
| 33 | GTAGTCTTGAGTTGGCTCCCTGTTTG | OBD168-131 | CAACTCGCTCCACTTTGGCATCGTAT |
| 34 | GCAGTAGATTTCTGTTGAGGTGATTA | OBD168-135 | CAGTTTTAGAATAAGTGCTATGTGGC |
| 35 | CTACTTCCCTTTCCATCCCACCC | OBD168-139 | CCCCAAACATTCTGCCAGGGTCC |
| 36 | GGGTCTAAGGAAGGTCAAGGCTGC | OBD168-143 | GCCCAAGCCCCTATTAGACCCAT |
| 37 | GCACCTGCTACCTGCTCCTTCCTATT | OBD168-147 | AACTGCGGAAAGGTCATAGCATAACC |
| 38 | GGTCTCACAATCACACTGCTGGG | OBD168-151 | CGACACTTCCCCATCTCCCCAAC |
| 39 | TTTTCAAATCCCTCTTGTGCCCTCAT | OBD168-155 | CCACAATCATCTCCCTTTCCCTCTGA |
| 40 | TCCTTCCTTTCCTACTCAGTGTGGAT | OBD168-159 | AGTCTACCTGAGGAGGCAACAGCCAT |
| 41 | CACTAAAGCACTCCAGTCAAGTCAGT | OBD168-163 | GGATGGCGAAATACCACGGGAAGCAT |
| 42 | GGGTAGGGAGAGGAAGGAAACGGATA | OBD168-167 | CAATAGTGCTTGTTCCTACCTCTCAC |
| 43 | GATTCTGTAGTCATCAGGAACACG | OBD168-171 | CAGGAGAGTGTGAGAGAGCCCGA |
| 44 | GTCTGCTTTGGAACATCAAGAGTGCT | OBD168-175 | GCTGACCTCCTCATCTTCACCCACAA |
| 45 | TGATTTCTGCCCAGCCGAGTGCTACA | OBD168-179 | ATACATTACCTTTCTGCTCAGCCAGC |
| 46 | GGACCATTGAGAGAATAACAGATA | OBD168-183 | GAAAGAGAATCACAAAGCATAAATA |
| 47 | GAGATGGATGGTGGCAATGGTTG | OBD168-187 | ATCTTGTTGGGAAATAAGGTCTTTGC |
| 48 | AACACAGTGGCTCTTCTCTTTGAA | OBD168-191 | CCCTGTCTCAAAACAAAACAAATA |
| 49 | GAGTGCTTGGCAGAGTGTGTGCT | OBD168-195 | GGTAGGTGATGGTCTGCCCTTCC |
| 50 | CCTTGCCTTGGCTTCTCTCGTAAAGA | OBD168-199 | CAACAGCCGCACACTCATTCACTCAT |

TABLE 1.a6

| | probe | Marker |
|---|---|---|
| 1 | ORF1_9_117652733_117662476_117782539_117784298_FF | OBD168-001.003 |
| 2 | ORF1_21_21135567_21136687_21190498_21197349_RR | OBD168-005.007 |
| 3 | ORF1_12_108190915_108198003_108364371_108366987_FR | OBD168-009.011 |
| 4 | ORF1_8_138185388_138192430_138316302_138317782_FF | OBD168-013.015 |
| 5 | ORF1_6_104807984_104813896_104858263_104859297_FR | OBD168-017.019 |
| 6 | ORF1_7_73838719_73840112_73998797_74003463_FF | OBD168-021.023 |
| 7 | ORF1_6_1490191_1492799_1612178_1614243_FR | OBD168-025.027 |
| 8 | ORF1_13_109624423_109630621_109869387_109876529_RF | OBD168-029.031 |
| 9 | ORF1_3_171788979_171791691_171971435_171976171_RF | OBD168-033.035 |
| 10 | ORF1_13_102842718_102847079_102927740_102931657_FR | OBD168-037.039 |
| 11 | ORF1_X_40971005_40973842_41006985_41010257_RF | OBD168-041.043 |
| 12 | ORF1_4_26084879_26086604_26290850_26293788_FR | OBD168-045.047 |
| 13 | ORF1_13_109869387_109876529_109901846_109907608_FF | OBD168-049.051 |
| 14 | ORF1_11_21505657_21511320_21625659_21628201_FR | OBD168-053.055 |
| 15 | ORF1_13_102842718_102847079_102867709_102871733_FF | OBD168-057.059 |
| 16 | ORF1_13_102842718_102847079_102952727_102953846_FF | OBD168-061.063 |
| 17 | ORF1_3_179490429_179499685_179696553_179701788_RF | OBD168-065.067 |
| 18 | ORF1_13_109869387_109876529_109994965_109996722_FR | OBD168-069.071 |
| 19 | ORF1_13_42949707_42958335_43086110_43092272_RF | OBD168-073.075 |
| 20 | ORF1_10_93375413_93378505_93410956_93413925_FR | OBD168-077.079 |
| 21 | ORF1_1_182254591_182258498_182271295_182272617_RR | OBD168-081.083 |
| 22 | ORF1_6_104807984_104813896_104859297_104866639_FF | OBD168-085.087 |
| 23 | ORF1_3_35637910_35639568_35705745_35720206_RF | OBD168-089.091 |
| 24 | ORF1_8_81130626_81134961_81156168_81161109_RF | OBD168-093.095 |
| 25 | ORF1_11_61243450_61246026_61388261_61391790_FR | OBD168-097.099 |
| 26 | ORF1_4_107675768_107680919_107825397_107833109_FF | OBD168-101.103 |
| 27 | ORF1_1_193456875_193464580_193519461_193528788_RR | OBD168-105.107 |
| 28 | ORF1_11_79134716_79152601_79219225_79225698_FR | OBD168-109.111 |
| 29 | ORF1_6_15282103_15283199_15362212_15366665_RR | OBD168-113.115 |
| 30 | ORF1_11_35101113_35109487_35250591_35252942_RR | OBD168-117.119 |
| 31 | ORF1_11_111144097_111148048_111222819_111226135_FF | OBD168-121.123 |
| 32 | ORF1_18_62522952_62526928_62633916_62637110_RR | OBD168-125.127 |
| 33 | ORF1_18_63796182_63806119_63902371_63912126_RF | OBD168-129.131 |
| 34 | ORF1_4_59658576_59668116_59701046_59706041_RF | OBD168-133.135 |
| 35 | ORF1_2_68857299_68862999_68948041_68951799_RF | OBD168-137.139 |
| 36 | ORF1_14_61751727_61758003_61810071_61812335_FR | OBD168-141.143 |
| 37 | ORF1_21_33072222_33074551_33276086_33283169_RF | OBD168-145.147 |
| 38 | ORF1_11_111222819_111226135_111261363_111265949_FR | OBD168-149.151 |
| 39 | ORF1_4_38671979_38673590_38888275_38891480_RF | OBD168-153.155 |
| 40 | ORF1_3_114010550_114016075_114047949_114051357_RF | OBD168-157.159 |
| 41 | ORF1_14_61490070_61495640_61751727_61758003_RF | OBD168-161.163 |
| 42 | ORF1_15_67887204_67889122_68052888_68053975_FR | OBD168-165.167 |
| 43 | ORF1_14_96740036_96741996_96820569_96823739_RF | OBD168-169.171 |
| 44 | ORF1_8_116637960_116642220_116837668_116843681_FR | OBD168-173.175 |
| 45 | ORF1_7_43764036_43765883_43792676_43795674_RR | OBD168-177.179 |
| 46 | ORF1_11_78798294_78806874_79036088_79041510_RF | OBD168-181.183 |
| 47 | ORF1_12_103575776_103578331_103663674_103664970_FR | OBD168-185.187 |
| 48 | ORF1_6_13375878_13378557_13523152_13526281_FF | OBD168-189.191 |
| 49 | ORF1_11_21505657_21511320_21751405_21757053_FR | OBD168-193.195 |
| 50 | ORF1_5_54531220_54532270_54669234_54674643_FF | OBD168-197.199 |

TABLE 1.b1

| | | | | |
|---|---|---|---|---|
| 51 | ORF1_chr1_161526936_161530260_161592992_161600164_RF_1 | 54097 | 99.6 | 2.682 |
| 52 | ORF1_chr17_59610792_59618647_59742829_59746633_FF_1 | 2321 | 88.77 | 3.732 |
| 53 | ORF1_chr1_59019583_59021645_59133061_59141815_RR_1 | 5747 | 77.65 | 2.939 |
| 54 | ORF1_chr1_59019583_59021645_59122227_59127200_RR_1 | 14439 | 167.9 | 2.225 |
| 55 | ORF1_chr8_2179427_2182848_2282002_2287140_RF_1 | 82548 | 140.9 | 2.302 |
| 56 | ORF1_chr12_31765908_31770394_31832455_31836936_RF_1 | 76089 | 135.3 | 3.146 |
| 57 | ORF1_chr7_127571782_127576218_127652961_127655625_RF_1 | 196 | 167.8 | 2.332 |
| 58 | ORF1_chr6_53297611_53301525_53330606_53336135_RF_1 | 4276 | 161.1 | 3.961 |
| 59 | ORF1_chr8_6544838_6545849_6690336_6691879_RR_1 | 10831 | 75.34 | 2.967 |
| 60 | ORF1_chr15_74848287_74855121_74957951_74959166_FR_1 | 2428 | 305.6 | 2.602 |
| 61 | ORF1_chr17_27180736_27185386_27333521_27337305_RR_1 | 25171 | 296 | 2.03 |
| 62 | ORF1_chr16_72743677_72746653_72988436_72990339_FR_1 | 110054 | 304.9 | 2.847 |
| 63 | ORF1_chr17_36180872_36185095_36216401_36221747_RR_1 | 20739 | 347.5 | 4.862 |
| 64 | ORF1_chr7_96316940_96320226_96464673_96468301_RR_1 | 1300 | 321 | 3.527 |
| 65 | ORF1_chr6_31267448_31269252_31345648_31347228_RF_1 | 51004 | 291.8 | 1.889 |
| 66 | ORF1_chr10_133409387_133412825_133526816_133529614_RF_1 | 40699 | 284.9 | 1.887 |
| 67 | ORF1_chr1_26201425_26205480_26333363_26336159_FR_1 | 121592 | 389.5 | 1.79 |
| 68 | ORF1_chr5_782236_785121_920775_927647_RF_1 | 110906 | 338 | 1.81 |
| 69 | ORF1_chr9_70567738_70570528_70702891_70712644_RF_1 | 4875 | 364.1 | 1.917 |
| 70 | ORF1_chr15_64931692_64934068_65028347_65030549_RF_1 | 82247 | 332.5 | 1.829 |
| 71 | ORF1_chr22_24772075_24777926_24949355_24952491_RR_1 | 12733 | 399 | 1.858 |
| 72 | ORF1_chr11_10637008_10641060_10653098_10661130_FR_1 | 136402 | 347 | 1.803 |
| 73 | ORF1_chr15_50065156_50069400_50125985_50128841_RR_1 | 2553 | 386.6 | 2.404 |
| 74 | ORF1_chr19_54789678_54792948_55035514_55037195_RF_1 | 99528 | 232.6 | 2.001 |
| 75 | ORF1_chr6_1571374_1573462_1612178_1614243_RR_1 | 1633 | 243.1 | 2.249 |
| 76 | ORF1_chr12_8943814_8945391_9117075_9121539_RR_1 | 77436 | 281.9 | 1.924 |
| 77 | ORF1_chr20_62682975_62688983_62704898_62706446_FF_1 | 112187 | 363.7 | 2.386 |
| 78 | ORF1_chr17_13014484_13018600_13163660_13166024_RF_1 | 128130 | 378 | 1.782 |
| 79 | ORF1_chr3_71509295_71510710_71611738_71613935_FF_1 | 130292 | 256.1 | 11.74 |
| 80 | ORF1_chr2_144423688_144428542_144476252_144486305_RF_1 | 4532 | 386.3 | 2.799 |
| 81 | ORF1_chr1_66194325_66201588_66444381_66456052_RR_1 | 130547 | 277.6 | 11.36 |
| 82 | ORF1_chr20_31723515_31724864_31865584_31867866_RR_1 | 1057 | 591.9 | 1.716 |
| 83 | ORF1_chr20_37222567_37225275_37490771_37494248_RR_1 | 119165 | 590.8 | 1.67 |
| 84 | ORF1_chr14_92536539_92540967_92697902_92700362_FR_1 | 29947 | 586.5 | 1.616 |
| 85 | ORF1_chr10_61891351_61894307_62056179_62058893_RR_1 | 104482 | 583.9 | 1.621 |
| 86 | ORF1_chr3_171803475_171808332_171888052_171894907_RF_1 | 17545 | 274.5 | 1.916 |
| 87 | ORF1_chr21_38766076_38770672_38858093_38859288_FR_1 | 25700 | 583.6 | 1.622 |
| 88 | ORF1_chr17_43300561_43304860_43323475_43325945_FF_1 | 91767 | 579.6 | 1.642 |
| 89 | ORF1_chr15_74686122_74687597_74772372_74779791_FR_1 | 107590 | 565.5 | 1.632 |
| 90 | ORF1_chr16_87389518_87390661_87484414_87491902_RF_1 | 106302 | 576.8 | 1.69 |
| 91 | ORF1_chr12_127036933_127040040_127282483_127288002_FR_1 | 90080 | 573.9 | 1.64 |
| 92 | ORF1_chr11_10637008_10641060_10723633_10730683_FF_1 | 115334 | 564.3 | 1.63 |
| 93 | ORF1_chr6_36408988_36410120_36693657_36702286_FF_1 | 104999 | 639.2 | 2.657 |
| 94 | ORF1_chr4_186172696_186175767_186190828_186192765_FF_1 | 132933 | 637.1 | 1.82 |
| 95 | ORF1_chr22_35115503_35116970_35401039_35404538_RF_1 | 108441 | 563.1 | 2.82 |
| 96 | ORF1_chr16_56676662_56679108_56730378_56735303_FR_1 | 107625 | 628.4 | 2.81 |
| 97 | ORF1_chr11_73240092_73243799_73403994_73408090_RR_1 | 115685 | 548.5 | 2.096 |
| 98 | ORF1_chr2_203737190_203742853_203893081_203899008_RF_1 | 134375 | 636.7 | 1.6 |
| 99 | ORF1_chr2_203893081_203899008_203933311_203940057_FR_1 | 97021 | 560.3 | 1.641 |
| 100 | ORF1_chr17_27180736_27185386_27282892_27289008_RR_1 | 90989 | 627.7 | 1.71 |

TABLE 1.b2

| | | | | |
|---|---|---|---|---|
| 51 | 0.1703 | 0.000004825 | Early | AAAAAACAATTATGTAATTGAAAACCCATCGAAGTACAAAAGTACCCAAAGGCTTTCAGA |
| 52 | 0.176 | 0.000003742 | Early | AAGAAAGTACCAGAAAGGTTATTATTAATCGATTTACAAAACTTGATGGTTAGTAATTTT |
| 53 | 0.1963 | 0.000002782 | Early | CTGGTTGACAATAACTATACAAAGAGTGTCGAAATATGTTCCTAAAATATTCAAAGTATT |
| 54 | 0.2382 | 0.00001519 | Early | CTGGTTGACAATAACTATACAAAGAGTGTCGACAGAGCTTAACAGCTGTTTCAAAGTCTT |
| 55 | 0.2433 | 0.00001034 | Early | CCAGACACAGGTAGTTCCTAAAATCTTCTCGAAGGCTTTTATTTAATTTGAGAATCATCA |
| 56 | 0.2671 | 0.000009462 | Early | GAACCCAGGAGACGGAGCTTGCAGTGAGTCGAGTATTATTTAGTCATAAAAAGGAATGAA |
| 57 | 0.2676 | 0.00001517 | Early | GTCTTCTATCAAATCAGATTTTATTTTCTCGAATCAGTTCTTGATTCTAATCTCCAATGT |
| 58 | 0.28 | 0.00001389 | Early | GTAGTATAATAATAAACTACCTGTTGATTCGATTTAATTATTAGTTATTGTTGTTAATCT |
| 59 | 0.3672 | 0.000002602 | Early | AATGACATAATTGGCCTCAAAACATATTTCGAGAGTTGGCTAGATTCATACACATAATTA |
| 60 | 0.3948 | 0.00005595 | Early | TCCTCTTTTTTTATATTTTTTTATTTTTTCGAAAAATGAATAAATTAATTAATTAAATAA |
| 61 | 0.4093 | 0.0000522 | Early | GGATCTAGAAAAGAGTATTTTCAATGTGTCGAATGTGTTGCAAATATTTTTCTAAGCTTA |
| 62 | 0.4135 | 0.00005567 | Early | GAGGCGGGTGGATCATGAGGTCGGAAGATCGATATGCAGGCTGGAACCCGCCAAACATTT |
| 63 | 0.4173 | 0.00007391 | Early | AATAAAGCACACAATAAATGTAATGGGCTCGACATTTTGAATATGTGGGATCCAAATCTG |
| 64 | 0.4183 | 0.00006225 | Early | TAAAAAAGATCGGATAATTAGGAAATTATCGATGATAAAATATATGTACCCCACTTAGAG |
| 65 | 0.4204 | 0.00005064 | Early | CCCTAAAGTCTGGGAAGGAAAGAGAGGCTCGATTCCTGTTTTCCCTTCTGCCTCCCTCGT |
| 66 | 0.4238 | 0.00004804 | Early | CAGCGTACATTTCTGAACATTGTGAACGTCGAGTCAGAAAAGATACTCAATAAGATTTTG |
| 67 | 0.4269 | 0.00009376 | Early | GGGTTTCACCATGTTGGTCAGGCTGGTCTCGAGGCCAGCCTGACCAACATGAAGAAACCC |
| 68 | 0.427 | 0.00006958 | Early | GAGAATCTGCTCAGAGCTGATTTCGTTATCGAGCAGTAGGAACTAGGCGTGTGTAGATTC |
| 69 | 0.4273 | 0.00008175 | Early | AGTTTGCCTATATTGGAATCATTAGCATTCGAATTGTGAAACAAAAACTGTTTGCTGGGC |
| 70 | 0.4309 | 0.00006717 | Early | GTTACTTGTCTGCAGTTACTTTCTTGAATCGAGGCAGCAGACAAGGTTCTGATCTTGGCT |
| 71 | 0.433 | 0.00009819 | Early | TGGTGCATTTATGATATACACTGTGTTATCGAAAAAAATTAAGTAATAATAATAATAGAG |
| 72 | 0.4333 | 0.00007369 | Early | GGACATAACTGTATTCTCTCTCTCTCTCTCGATGAGCTTAAAGTGGTGTTCTAGGACCAG |
| 73 | 0.4349 | 0.00009244 | Early | AATATGTATAAATTACAAAATGGCTAAATCGATATCTGCCCTAACAAGAATCCTTCTAGT |
| 74 | 0.4363 | 0.00003092 | Early | ATTCTGAAGAAATCAGGAAATAATGCATTCGATAGATAATAGATAGAAATATGCAGAAAG |
| 75 | 0.4368 | 0.00003404 | Early | ATAGTATATTATCTAAAATGTCAAATTTTCGAAACTAAAAAAAAAAAATCCAATTAAAAA |
| 76 | 0.4419 | 0.00004696 | Early | GTCGTGCAGTGGTGATGATCTCCTGCAATCGAAGAACAAAGAGATGAAAAGAGCTTTCTA |
| 77 | 0.4427 | 0.00008155 | Early | GCTGGAAAATCAGGCTCATCACAAAAGCTCGAGACCCCCACAAAGAGGAAGTCCACAGCA |
| 78 | 0.4463 | 0.00008854 | Early | CCTCAGTCTTTCTTTGTTTTCCAAGACCTCGAGGTATTTTTCCTAATGAAAAACAAAGAA |
| 79 | 0.4485 | 0.00003814 | Early | CTGCAATGTTCAACTAAGCTCTGTCCAATCGAGGTTGTTAGTTTTGAAGATTGAGGAAGG |
| 80 | 0.4492 | 0.00009229 | Early | AAAAATGTGTATTTCTACATTTTTTTGGTCGAATGAATAGAATGACAATAGGTTTTTATT |
| 81 | 0.4579 | 0.00004543 | Early | AACATAGCATATTCAACTAAGCAATTCATCGAAGTATCCTTATATTTTAGATTAACATAT |
| 82 | 0.4628 | 0.0002066 | Early | GGCTCGGCGGGTACCCGGCCTAGTGAGCTCGAGAAACTGTCTTAACATTTTTATATTCAA |
| 83 | 0.4687 | 0.0002059 | Early | AGCTCTGGCAGTTTTCTGAAATACCATATCGAGCTGTGGTGATTACTTTCTCCCTGAAAC |
| 84 | 0.4699 | 0.0002031 | Early | TCACTAGCAGATTCCTGCATTCTTTCAGTCGAAGAGTGAACCTTGATGTATGCAGATTTT |
| 85 | 0.4738 | 0.0002014 | Early | ATGTCTTAATATTATTATAATCATTGTTTCGATATGCCAAGTTCCCTGTCTGCCTACAGT |
| 86 | 0.4814 | 0.00004434 | Early | GGTCGTTGAGGACAATTTACAAATTGGCTCGATGGTGTAAATATTCCTGGAAGGCCAAGG |
| 87 | 0.4814 | 0.0002012 | Early | CACTTTTTACACATGAGAGGTGAGGATATCGACCAAGAGATCGCGAGATGGGAGCAGAGA |
| 88 | 0.4833 | 0.0001986 | Early | AACAACTGCTGCAAGAGGGGCCGTTTGTTCGATGCTACTTTTAATCCCAGAACAAGTATT |
| 89 | 0.4868 | 0.0001897 | Early | GGGTTTCACCATGTTGGCCAGACTGATCTCGAGACGATCCCGGCCAACATGGTGAAACCC |
| 90 | 0.4874 | 0.0001969 | Early | CTGCAGCCCGTAGTTGTGGATGATGGAGTCGATTTTTTCCCTTCTTCTTGCATTTGGAAC |
| 91 | 0.4915 | 0.000195 | Early | GAGAGAGAGAGAGAAAGAGACGAAGATTTCGACCACTCTTGAGCTGTGTGATGTTGATCT |
| 92 | 0.4938 | 0.000189 | Early | GGACATAACTGTATTCTCTCTCTCTCTCTCGAGGAAGGTTCTGAGGAATTGAAGCCCACT |
| 93 | 0.4948 | 0.0002384 | Early | GGCAGGTGGCTCACCTGAGGTCGGGAGTTCGAAGTCTGTTTCAGATGAAGTTTTAAGATC |
| 94 | 0.499 | 0.0002369 | Early | ACGGAGTGGAAGGAGCTGCAACTTTCCATCGAGTTCGCCCTCCCTGACAGGGAGTTGTAA |
| 95 | 0.5011 | 0.0001882 | Early | AAGTTAGATGCAAAGGCCTGACTGTGTTTCGAGCATAGATCCCAGCCCCAGATCCCACAG |
| 96 | 0.5015 | 0.000231 | Early | TCCAGCTATCTGCTCAGGGAATTGTTATTCGAGCCGCCCCCGAGGCTTGGAGTCGGGCTG |
| 97 | 0.5057 | 0.0001792 | Early | AGGAGTGGCCAGGCAGAGCCAAGTCCTATCGAGTGGCTCTGTCACTTATTAGCAAGTCAC |
| 98 | 0.506 | 0.0002366 | Early | AATCTGTCTCTCTCTTACTCCCTCTCTCTCGAAAATACTGTACTTTGGTTGATTTTTAAG |
| 99 | 0.5062 | 0.0001865 | Early | AATCTGTCTCTCTCTTACTCCCTCTCTCTCGATGGGTATGGAAGCAAAGGCCTGAAGAAT |
| 100 | 0.5082 | 0.0002305 | Early | GGATCTAGAAAAGAGTATTTTCAATGTGTCGACACAGATGATTAGGGCATCTTCTGCTCT |

TABLE 1.b3

| | | | | | | | | | | |
|---|---|---|---|---|---|---|---|---|---|---|
| 51 | chr1 | 161526938 | 161526967 | 161600133 | 161600162 | chr1 | 161526938 | 161530937 | 161596163 | 161600162 |
| 52 | chr17 | 59618616 | 59618645 | 59746602 | 59746631 | chr17 | 59614646 | 59618645 | 59742632 | 59746631 |
| 53 | chr1 | 59019585 | 59019614 | 59133063 | 59133092 | chr1 | 59019585 | 59023584 | 59133063 | 59137062 |
| 54 | chr1 | 59019585 | 59019614 | 59122229 | 59122258 | chr1 | 59019585 | 59023584 | 59122229 | 59126228 |
| 55 | chr8 | 2179429 | 2179458 | 2287109 | 2287138 | chr8 | 2179429 | 2183428 | 2283139 | 2287138 |
| 56 | chr12 | 31765910 | 31765939 | 31836905 | 31836934 | chr12 | 31765910 | 31769909 | 31832935 | 31836934 |
| 57 | chr7 | 127571784 | 127571813 | 127655594 | 127655623 | chr7 | 127571784 | 127575783 | 127651624 | 127655623 |
| 58 | chr6 | 53297613 | 53297642 | 53336104 | 53336133 | chr6 | 53297613 | 53301612 | 53332134 | 53336133 |
| 59 | chr8 | 6544840 | 6544869 | 6690338 | 6690367 | chr8 | 6544840 | 6548839 | 6690338 | 6694337 |
| 60 | chr15 | 74855090 | 74855119 | 74957953 | 74957982 | chr15 | 74851120 | 74855119 | 74957953 | 74961952 |
| 61 | chr17 | 27180738 | 27180767 | 27333523 | 27333552 | chr17 | 27180738 | 27184737 | 27333523 | 27337522 |
| 62 | chr16 | 72746622 | 72746651 | 72988438 | 72988467 | chr16 | 72742652 | 72746651 | 72988438 | 72992437 |
| 63 | chr17 | 36180874 | 36180903 | 36216403 | 36216432 | chr17 | 36180874 | 36184873 | 36216403 | 36220402 |
| 64 | chr7 | 96316942 | 96316971 | 96464675 | 96464704 | chr7 | 96316942 | 96320941 | 96464675 | 96468674 |
| 65 | chr6 | 31267450 | 31267479 | 31347197 | 31347226 | chr6 | 31267450 | 31271449 | 31343227 | 31347226 |
| 66 | chr10 | 133409389 | 133409418 | 133529583 | 133529612 | chr10 | 133409389 | 133413388 | 133525613 | 133529612 |
| 67 | chr1 | 26205449 | 26205478 | 26333365 | 26333394 | chr1 | 26201479 | 26205478 | 26333365 | 26337364 |
| 68 | chr5 | 782238 | 782267 | 927616 | 927645 | chr5 | 782238 | 786237 | 923646 | 927645 |
| 69 | chr9 | 70567740 | 70567769 | 70712613 | 70712642 | chr9 | 70567740 | 70571739 | 70708643 | 70712642 |
| 70 | chr15 | 64931694 | 64931723 | 65030518 | 65030547 | chr15 | 64931694 | 64935693 | 65026548 | 65030547 |
| 71 | chr22 | 24772077 | 24772106 | 24949357 | 24949386 | chr22 | 24772077 | 24776076 | 24949357 | 24953356 |
| 72 | chr11 | 10641029 | 10641058 | 10653100 | 10653129 | chr11 | 10637059 | 10641058 | 10653100 | 10657099 |
| 73 | chr15 | 50065158 | 50065187 | 50125987 | 50126016 | chr15 | 50065158 | 50069157 | 50125987 | 50129986 |
| 74 | chr19 | 54789680 | 54789709 | 55037164 | 55037193 | chr19 | 54789680 | 54793679 | 55033194 | 55037193 |
| 75 | chr6 | 1571376 | 1571405 | 1612180 | 1612209 | chr6 | 1571376 | 1575375 | 1612180 | 1616179 |
| 76 | chr12 | 8943816 | 8943845 | 9117077 | 9117106 | chr12 | 8943816 | 8947815 | 9117077 | 9121076 |
| 77 | chr20 | 62688952 | 62688981 | 62706415 | 62706444 | chr20 | 62684982 | 62688981 | 62702445 | 62706444 |
| 78 | chr17 | 13014486 | 13014515 | 13165993 | 13166022 | chr17 | 13014486 | 13018485 | 13162023 | 13166022 |
| 79 | chr3 | 71510679 | 71510708 | 71613904 | 71613933 | chr3 | 71506709 | 71510708 | 71609934 | 71613933 |
| 80 | chr2 | 144423690 | 144423719 | 144486274 | 144486303 | chr2 | 144423690 | 144427689 | 144482304 | 144486303 |
| 81 | chr1 | 66194327 | 66194356 | 66444383 | 66444412 | chr1 | 66194327 | 66198326 | 66444383 | 66448382 |
| 82 | chr20 | 31723517 | 31723546 | 31865586 | 31865615 | chr20 | 31723517 | 31727516 | 31865586 | 31869585 |
| 83 | chr20 | 37222569 | 37222598 | 37490773 | 37490802 | chr20 | 37222569 | 37226568 | 37490773 | 37494772 |
| 84 | chr14 | 92540936 | 92540965 | 92697904 | 92697933 | chr14 | 92536966 | 92540965 | 92697904 | 92701903 |
| 85 | chr10 | 61891353 | 61891382 | 62056181 | 62056210 | chr10 | 61891353 | 61895352 | 62056181 | 62060180 |
| 86 | chr3 | 171803477 | 171803506 | 171894876 | 171894905 | chr3 | 171803477 | 171807476 | 171890906 | 171894905 |
| 87 | chr21 | 38770641 | 38770670 | 38858095 | 38858124 | chr21 | 38766671 | 38770670 | 38858095 | 38862094 |
| 88 | chr17 | 43304829 | 43304858 | 43325914 | 43325943 | chr17 | 43300859 | 43304858 | 43321944 | 43325943 |
| 89 | chr15 | 74687566 | 74687595 | 74772374 | 74772403 | chr15 | 74683596 | 74687595 | 74772374 | 74776373 |
| 90 | chr16 | 87389520 | 87389549 | 87491871 | 87491900 | chr16 | 87389520 | 87393519 | 87487901 | 87491900 |
| 91 | chr12 | 127040009 | 127040038 | 127282485 | 127282514 | chr12 | 127036039 | 127040038 | 127282485 | 127286484 |
| 92 | chr11 | 10641029 | 10641058 | 10730652 | 10730681 | chr11 | 10637059 | 10641058 | 10726682 | 10730681 |
| 93 | chr6 | 36410089 | 36410118 | 36702255 | 36702284 | chr6 | 36406119 | 36410118 | 36698285 | 36702284 |
| 94 | chr4 | 186175736 | 186175765 | 186192734 | 186192763 | chr4 | 186171766 | 186175765 | 186188764 | 186192763 |
| 95 | chr22 | 35115505 | 35115534 | 35404507 | 35404536 | chr22 | 35115505 | 35119504 | 35400537 | 35404536 |
| 96 | chr16 | 56679077 | 56679106 | 56730380 | 56730409 | chr16 | 56675107 | 56679106 | 56730380 | 56734379 |
| 97 | chr11 | 73240094 | 73240123 | 73403996 | 73404025 | chr11 | 73240094 | 73244093 | 73403996 | 73407995 |
| 98 | chr2 | 203737192 | 203737221 | 203898977 | 203899006 | chr2 | 203737192 | 203741191 | 203895007 | 203899006 |
| 99 | chr2 | 203898977 | 203899006 | 203933313 | 203933342 | chr2 | 203895007 | 203899006 | 203933313 | 203937312 |
| 100 | chr17 | 27180738 | 27180767 | 27282894 | 27282923 | chr17 | 27180738 | 27184737 | 27282894 | 27286893 |

TABLE 1.b4

| | | |
|---|---|---|
| 51 | ORF1_1_161526936_161530260_161592992_161600164_RF | OBD168-201 |
| 52 | ORF1_17_59610792_59618647_59742829_59746633_FF | OBD168-205 |
| 53 | ORF1_1_59019583_59021645_59133061_59141815_RR | OBD168-209 |
| 54 | ORF1_1_59019583_59021645_59122227_59127200_RR | OBD168-213 |
| 55 | ORF1_8_2179427_2182848_2282002_2287140_RF | OBD168-217 |
| 56 | ORF1_12_31765908_31770394_31832455_31836936_RF | OBD168-221 |
| 57 | ORF1_7_127571782_127576218_127652961_127655625_RF | OBD168-225 |
| 58 | ORF1_6_53297611_53301525_53330606_53336135_RF | OBD168-229 |
| 59 | ORF1_8_6544838_6545849_6690336_6691879_RR | OBD168-233 |
| 60 | ORF1_15_74848287_74855121_74957951_74959166_FR | OBD168-237 |
| 61 | ORF1_17_27180736_27185386_27333521_27337305_RR | OBD168-241 |
| 62 | ORF1_16_72743677_72746653_72988436_72990339_FR | OBD168-245 |
| 63 | ORF1_17_36180872_36185095_36216401_36221747_RR | OBD168-249 |
| 64 | ORF1_7_96316940_96320226_96464673_96468301_RR | OBD168-253 |
| 65 | ORF1_6_31267448_31269252_31345648_31347228_RF | OBD168-257 |
| 66 | ORF1_10_133409387_133412825_133526816_133529614_RF | OBD168-261 |
| 67 | ORF1_1_26201425_26205480_26333363_26336159_FR | OBD168-265 |
| 68 | ORF1_5_782236_785121_920775_927647_RF | OBD168-269 |
| 69 | ORF1_9_70567738_70570528_70702891_70712644_RF | OBD168-273 |
| 70 | ORF1_15_64931692_64934068_65028347_65030549_RF | OBD168-277 |
| 71 | ORF1_22_24772075_24777926_24949355_24952491_RR | OBD168-281 |
| 72 | ORF1_11_10637008_10641060_10653098_10661130_FR | OBD168-285 |
| 73 | ORF1_15_50065156_50069400_50125985_50128841_RR | OBD168-289 |
| 74 | ORF1_19_54789678_54792948_55035514_55037195_RF | OBD168-293 |
| 75 | ORF1_6_1571374_1573462_1612178_1614243_RR | OBD168-297 |
| 76 | ORF1_12_8943814_8945391_9117075_9121539_RR | OBD168-301 |
| 77 | ORF1_20_62682975_62688983_62704898_62706446_FF | OBD168-305 |
| 78 | ORF1_17_13014484_13018600_13163660_13166024_RF | OBD168-309 |
| 79 | ORF1_3_71509295_71510710_71611738_71613935_FF | OBD168-313 |
| 80 | ORF1_2_144423688_144428542_144476252_144486305_RF | OBD168-317 |
| 81 | ORF1_1_66194325_66201588_66444381_66456052_RR | OBD168-321 |
| 82 | ORF1_20_31723515_31724864_31865584_31867866_RR | OBD168-325 |
| 83 | ORF1_20_37222567_37225275_37490771_37494248_RR | OBD168-329 |
| 84 | ORF1_14_92536539_92540967_92697902_92700362_FR | OBD168-333 |
| 85 | ORF1_10_61891351_61894307_62056179_62058893_RR | OBD168-337 |
| 86 | ORF1_3_171803475_171808332_171888052_171894907_RF | OBD168-341 |
| 87 | ORF1_21_38766076_38770672_38858093_38859288_FR | OBD168-345 |
| 88 | ORF1_17_43300561_43304860_43323475_43325945_FF | OBD168-349 |
| 89 | ORF1_15_74686122_74687597_74772372_74779791_FR | OBD168-353 |
| 90 | ORF1_16_87389518_87390661_87484414_87491902_RF | OBD168-357 |
| 91 | ORF1_12_127036933_127040040_127282483_127288002_FR | OBD168-361 |
| 92 | ORF1_11_10637008_10641060_10723633_10730683_FF | OBD168-365 |
| 93 | ORF1_6_36408988_36410120_36693657_36702286_FF | OBD168-369 |
| 94 | ORF1_4_186172696_186175767_186190828_186192765_FF | OBD168-373 |
| 95 | ORF1_22_35115503_35116970_35401039_35404538_RF | OBD168-377 |
| 96 | ORF1_16_56676662_56679108_56730378_56735303_FR | OBD168-381 |
| 97 | ORF1_11_73240092_73243799_73403994_73408090_RR | OBD168-385 |
| 98 | ORF1_2_203737190_203742853_203893081_203899008_RF | OBD168-389 |
| 99 | ORF1_2_203893081_203899008_203933311_203940057_FR | OBD168-393 |
| 100 | ORF1_17_27180736_27185386_27282892_27289008_RR | OBD168-397 |

TABLE 1.b5

| | | | |
|---|---|---|---|
| 51 | CCAGCCAATCACAAGCAGCCTAC | OBD168-203 | CTCCTTCAGCACCCACCCCAACA |
| 52 | CTTTCATCCTTTATTGTATTGGTGCG | OBD168-207 | GGATTACAGGCGTGAGCCAACAT |
| 53 | TCCTCACTCTCCTGCCACTCCCT | OBD168-211 | CCAAGTAAGTGGAGGCTGGCTCT |
| 54 | CTCACTCTCCTGCCACTCCCTGC | OBD168-215 | CAAGAAAATCCAGATGCTCAAAACAC |
| 55 | TGTGGACAGATGCTGAGACCCTC | OBD168-219 | GAGACAGGGCTTCAGAGTCACAGG |
| 56 | TACTCGGGAGGCTGAGGCAGAAT | OBD168-223 | CTCTGTGGATTTACCTATTTTGGACA |
| 57 | GGAGGAAGTGGACAGTAAGTTGATAC | OBD168-227 | ATGGACAGGCAGAGGTGGTGACAATG |
| 58 | CCATCCACCCTGCCTTTCATAAGAGA | OBD168-231 | GGCAGGAAAGTGGCTTAGGACCAAAA |
| 59 | AGTGAGACTACTGACCAAAGAATC | OBD168-235 | GCTGAAACCCCAAGATAAAACTAT |
| 60 | GGGTGGTGAGGGTAGAGACTTAC | OBD168-239 | CCCAGGGCTTCAACTCCCCATCT |
| 61 | CAATGGGCTCTGAAATGTCACTTC | OBD168-243 | CATCAAAATGTCGTGTGGTGTATCAA |
| 62 | GGAGGTATGAGAAGGCTATTGTAGGA | OBD168-247 | TCAGAGGAAGGCTGGAGTGAATCCCT |
| 63 | CTGATTCTATGTTATGGTGAGTTGTG | OBD168-251 | CCATCATCGGTTGAATCCACAGAT |
| 64 | GCCTGGGTGACAGAGCAAGACTC | OBD168-255 | AACCTTAGCCCCACAATCAGAGC |
| 65 | AGGATTGGAACTGTTAGCCCCAT | OBD168-259 | CTCTGATGGCTGTGCTGAGGACA |
| 66 | GCCTGCCCTGCGGTTTCAAAGTAGAT | OBD168-263 | GCAAAACACACATTCTTCTCATCGGC |
| 67 | AGCCTTCCGAGTAGGTGGGAGTA | OBD168-267 | ACTCTCCTGCCTCAGCCTCTTGA |
| 68 | TTTTCTGGCAGCCTCTCCTTCCG | OBD168-271 | AACCCTCAGCCAGGAGCCAGGAT |
| 69 | GTGCCAGAACACGAGAGACACATAGG | OBD168-275 | AAACTCCCTAAGTCCGTAAATACCCC |
| 70 | AGGACAGAGCAGAGGTGGGACTC | OBD168-279 | TCACAGGGCTCAGCAGTGGAGAA |
| 71 | TGTCCTGTTGCTCATAGAAGTTGCTA | OBD168-283 | CACGGTAATGATACTAATGAAGGCTA |
| 72 | GGATAAAGCACAGGATGCCCCAG | OBD168-287 | CACACTCCACCACTCAGAGCAGG |
| 73 | CCAAATGGTCTGGTTCTGGGAATCTT | OBD168-291 | GAAGCCTGTTTGGACAAGGGACACCT |
| 74 | GATGCTGTCCCTTTAGTGGTCAA | OBD168-295 | CACTTTGTGTCCTGTTCATAACTTTC |
| 75 | GGGCTTCAGGGAAAATCAGTATTCTG | OBD168-299 | TTTTCTTCGTGCTGGTGCTGGTGAGC |
| 76 | CATTTTCCTTGTCCCTCCTCAGGTAA | OBD168-303 | TGCCGTGATTCTTCTCCCTCCTTTGC |
| 77 | CAGGCTGGTGGGAAGGAGACATT | OBD168-307 | TGGCAGAGGGACAGACTCCAGTG |
| 78 | TGTCTCTTTAGTCCCCTGTGGTCTGT | OBD168-311 | TGTTTCCTTTTGTCTCCTCCACTGGC |
| 79 | TAGTGTCCTCCCAGTGCTGTAGTGAG | OBD168-315 | TCAGGGAGAACGCTTTTCCTTGATTC |
| 80 | TATTTGGCTGAAAAGCACTCTTGC | OBD168-319 | ACTACTTCTACTCAACTCATCCAT |
| 81 | GGCGATGGACACCCTTGTAACCTTTC | OBD168-323 | CCCCACACTTTCAGATGTCAGTAGTA |
| 82 | GCTGGGCTCGCTGATTGGCTGCG | OBD168-327 | GCCCCTGAAAAGAGCCATTTGAA |
| 83 | GGAATAGAGATAACGGGCTGGGC | OBD168-331 | GGGTGGGTGACAAAGGAGAGAGG |
| 84 | GCCAAGGGTCGGTTGAGTCACAG | OBD168-335 | GTAGTCTGCGTTCCACCTTGGGA |
| 85 | GAAACATAGTGAGAAGACAGGCATTG | OBD168-339 | CTCCAGATGTTCTTCCTTCCACACTG |
| 86 | CACCAGTTACCACCAACATACATTAT | OBD168-343 | GGTCAGGCTGGTCTTGAACTCCT |
| 87 | GACCGATTGCTCCTCTTTAGCCC | OBD168-347 | TCCCCTCATCACTGCGGTCCCTA |
| 88 | GGGATTTGGACGAGAGACACAGGATG | OBD168-351 | CGGTTTGAACAGAGTTGTGGATAGCA |
| 89 | CCTCCCAGGTTCAATCAGGCAAT | OBD168-355 | CTCCTACCTCATCCTCCCGAGTA |
| 90 | CAGGAACTCATCGCCCGTGCGGC | OBD168-359 | TTCTCTTGCTGGACAACTGTTCC |
| 91 | GAGCGAGAGCAGGAGTGAGTGAG | OBD168-363 | AGACAGGCTGAGAGGTTTGGCTG |
| 92 | GGATAAAGCACAGGATGCCCCAG | OBD168-367 | CTCCACCTTCCCCACTCAAGCAG |
| 93 | TGCCTGTAATCCCAGCACTTTGG | OBD168-371 | CACCACTGCGGTTTTACATTTATGAT |
| 94 | AAGAAACAAAGCGAGGCAAGGTGACG | OBD168-375 | AGGCAGACTTCTTTGGAGGGTGTCAA |
| 95 | GTGGTTACAACATACCCCTTCCCC | OBD168-379 | CCTGCGGGACAAATCCAACCCAC |
| 96 | GCGAAGTGTCCAGCATCGTCCTG | OBD168-383 | AGTGGACGAATGAAAGGCACCCC |
| 97 | CAAGACAGAGGCAAGCCCAGGAA | OBD168-387 | TCCCAGAACCCTGCTGCCTCCAT |
| 98 | GCCTCCAAAAGTGTATGAGCCAA | OBD168-391 | CAACAGTTCTTCAGCACCCAAGTA |
| 99 | GCCTCCAAAAGTGTATGAGCCAA | OBD168-395 | TGCTTCCCCTCCTCACCACTCTA |
| 100 | CCGCATCGCAAAGGATTTTCTCAAAG | OBD168-399 | GAAAGTGGGATGGGAGAACAGAGCCA |

TABLE 1.b6

| | | |
|---|---|---|
| 51 | ORF1_1_161526936_161530260_161592992_161600164_RF | OBD168-201.203 |
| 52 | ORF1_17_59610792_59618647_59742829_59746633_FF | OBD168-205.207 |
| 53 | ORF1_1_59019583_59021645_59133061_59141815_RR | OBD168-209.211 |
| 54 | ORF1_1_59019583_59021645_59122227_59127200_RR | OBD168-213.215 |
| 55 | ORF1_8_2179427_2182848_2282002_2287140_RF | OBD168-217.219 |
| 56 | ORF1_12_31765908_31770394_31832455_31836936_RF | OBD168-221.223 |
| 57 | ORF1_7_127571782_127576218_127652961_127655625_RF | OBD168-225.227 |
| 58 | ORF1_6_53297611_53301525_53330606_53336135_RF | OBD168-229.231 |
| 59 | ORF1_8_6544838_6545849_6690336_6691879_RR | OBD168-233.235 |
| 60 | ORF1_15_74848287_74855121_74957951_74959166_FR | OBD168-237.239 |
| 61 | ORF1_17_27180736_27185386_27333521_27337305_RR | OBD168-241.243 |
| 62 | ORF1_16_72743677_72746653_72988436_72990339_FR | OBD168-245.247 |
| 63 | ORF1_17_36180872_36185095_36216401_36221747_RR | OBD168-249.251 |
| 64 | ORF1_7_96316940_96320226_96464673_96468301_RR | OBD168-253.255 |
| 65 | ORF1_6_31267448_31269252_31345648_31347228_RF | OBD168-257.259 |
| 66 | ORF1_10_133409387_133412825_133526816_133529614_RF | OBD168-261.263 |
| 67 | ORF1_1_26201425_26205480_26333363_26336159_FR | OBD168-265.267 |
| 68 | ORF1_5_782236_785121_920775_927647_RF | OBD168-269.271 |
| 69 | ORF1_9_70567738_70570528_70702891_70712644_RF | OBD168-273.275 |
| 70 | ORF1_15_64931692_64934068_65028347_65030549_RF | OBD168-277.279 |
| 71 | ORF1_22_24772075_24777926_24949355_24952491_RR | OBD168-281.283 |
| 72 | ORF1_11_10637008_10641060_10653098_10661130_FR | OBD168-285.287 |
| 73 | ORF1_15_50065156_50069400_50125985_50128841_RR | OBD168-289.291 |
| 74 | ORF1_19_54789678_54792948_55035514_55037195_RF | OBD168-293.295 |
| 75 | ORF1_6_1571374_1573462_1612178_1614243_RR | OBD168-297.299 |
| 76 | ORF1_12_8943814_8945391_9117075_9121539_RR | OBD168-301.303 |
| 77 | ORF1_20_62682975_62688983_62704898_62706446_FF | OBD168-305.307 |
| 78 | ORF1_17_13014484_13018600_13163660_13166024_RF | OBD168-309.311 |
| 79 | ORF1_3_71509295_71510710_71611738_71613935_FF | OBD168-313.315 |
| 80 | ORF1_2_144423688_144428542_144476252_144486305_RF | OBD168-317.319 |
| 81 | ORF1_1_66194325_66201588_66444381_66456052_RR | OBD168-321.323 |
| 82 | ORF1_20_31723515_31724864_31865584_31867866_RR | OBD168-325.327 |
| 83 | ORF1_20_37222567_37225275_37490771_37494248_RR | OBD168-329.331 |
| 84 | ORF1_14_92536539_92540967_92697902_92700362_FR | OBD168-333.335 |
| 85 | ORF1_10_61891351_61894307_62056179_62058893_RR | OBD168-337.339 |
| 86 | ORF1_3_171803475_171808332_171888052_171894907_RF | OBD168-341.343 |
| 87 | ORF1_21_38766076_38770672_38858093_38859288_FR | OBD168-345.347 |
| 88 | ORF1_17_43300561_43304860_43323475_43325945_FF | OBD168-349.351 |
| 89 | ORF1_15_74686122_74687597_74772372_74779791_FR | OBD168-353.355 |
| 90 | ORF1_16_87389518_87390661_87484414_87491902_RF | OBD168-357.359 |
| 91 | ORF1_12_127036933_127040040_127282483_127288002_FR | OBD168-361.363 |
| 92 | ORF1_11_10637008_10641060_10723633_10730683_FF | OBD168-365.367 |
| 93 | ORF1_6_36408988_36410120_36693657_36702286_FF | OBD168-369.371 |
| 94 | ORF1_4_186172696_186175767_186190828_186192765_FF | OBD168-373.375 |
| 95 | ORF1_22_35115503_35116970_35401039_35404538_RF | OBD168-377.379 |
| 96 | ORF1_16_56676662_56679108_56730378_56735303_FR | OBD168-381.383 |
| 97 | ORF1_11_73240092_73243799_73403994_73408090_RR | OBD168-385.387 |
| 98 | ORF1_2_203737190_203742853_203893081_203899008_RF | OBD168-389.391 |
| 99 | ORF1_2_203893081_203899008_203933311_203940057_FR | OBD168-393.395 |
| 100 | ORF1_17_27180736_27185386_27282892_27289008_RR | OBD168-397.399 |

TABLE 2.a1

| | probe | gene.index | RP/Rsum | FC:(class1/class2) |
|---|---|---|---|---|
| 1 | ORF1_chr15_94287688_94292746_94340208_94346941_FR_1 | 92819 | 39.39 | 3.235 |
| 2 | ORF1_chr4_102312731_102315549_102386632_102391817_FR_1 | 109353 | 95.41 | 2.368 |
| 3 | ORF1_chr8_38716162_38718294_38738341_38742581_RR_1 | 122864 | 89.18 | 2.377 |
| 4 | ORF1_chr5_132458507_132464714_132517598_132521351_RF_1 | 83272 | 94.59 | 2.287 |
| 5 | ORF1_chr12_96463989_96466636_96618278_96624162_RR_1 | 108826 | 87.53 | 2.328 |
| 6 | ORF1_chr11_79036088_79041510_79269569_79271288_FF_1 | 124955 | 81.79 | 2.53 |
| 7 | ORF1_chr1_11226037_11228251_11272812_11274518_RR_1 | 93778 | 112.6 | 2.208 |
| 8 | ORF1_chr2_159283360_159285194_159326592_159328925_FR_1 | 75100 | 149 | 2.024 |
| 9 | ORF1_chr8_90002099_90011473_90148439_90150313_FR_1 | 104201 | 153.5 | 2.132 |
| 10 | ORF1_chr3_113947063_113952443_113975285_113978229_RR_1 | 137463 | 160.9 | 2.052 |
| 11 | ORF1_chr3_149818709_149821071_150005759_150009319_FR_1 | 87373 | 148.9 | 2.093 |
| 12 | ORF1_chr5_60597760_60604069_60738056_60741292_FR_1 | 107779 | 160.8 | 2.049 |
| 13 | ORF1_chr6_53534187_53536920_53639583_53651122_RF_1 | 132284 | 183.6 | 2.105 |
| 14 | ORF1_chr5_88269888_88274763_88365192_88376199_FR_1 | 114376 | 192.3 | 1.96 |
| 15 | ORF1_chr9_76572019_76577216_76769644_76771052_RF_1 | 3234 | 181.2 | 1.994 |
| 16 | ORF1_chr18_68711389_68714330_68911644_68918950_RF_1 | 101101 | 203.8 | 1.951 |
| 17 | ORF1_chr5_134464509_134469933_134674132_134676030_RR_1 | 65542 | 191.6 | 1.946 |
| 18 | ORF1_chr8_92469388_92478361_92510934_92515600_RR_1 | 89074 | 203 | 1.92 |
| 19 | ORF1_chr10_54058576_54060371_54213283_54215754_FR_1 | 121042 | 200.8 | 2.123 |
| 20 | ORF1_chr1_109159473_109163477_109318126_109321152_RF_1 | 132421 | 199.6 | 1.915 |
| 21 | ORF1_chr12_9614159_9624874_9729793_9733228_FF_1 | 85149 | 211.9 | 1.939 |
| 22 | ORF1_chr14_89417652_89420597_89636182_89641783_RF_1 | 113983 | 214.7 | 1.908 |
| 23 | ORF1_chr1_155206612_155209922_155288575_155290398_FR_1 | 6515 | 248.8 | 1.853 |
| 24 | ORF1_chr5_173450236_173452725_173479240_173485435_RF_1 | 129576 | 244.7 | 1.907 |
| 25 | ORF1_chr3_43246875_43252830_43297212_43303273_FR_1 | 2491 | 241.1 | 2.104 |
| 26 | ORF1_chr2_118090940_118096647_118214615_118218048_RR_1 | 106991 | 247.9 | 1.981 |
| 27 | ORF1_chr5_96799181_96801649_96813450_96815948_RF_1 | 85793 | 244.4 | 1.855 |
| 28 | ORF1_chr3_43094298_43105527_43246875_43252830_RF_1 | 24515 | 257.1 | 2.065 |
| 29 | ORF1_chr9_16574863_16577692_16721839_16729333_FF_1 | 91315 | 267.3 | 1.816 |
| 30 | ORF1_chr3_107414954_107426087_107525934_107527641_RF_1 | 59128 | 278.4 | 1.804 |
| 31 | ORF1_chr12_13090807_13095110_13190440_13193042_FR_1 | 35176 | 283.4 | 1.815 |
| 32 | ORF1_chr5_35271024_35276749_35319374_35321203_RR_1 | 93369 | 321.1 | 1.766 |
| 33 | ORF1_chr13_109649213_109651298_109745462_109746722_RR_1 | 73079 | 329.8 | 2.135 |
| 34 | ORF1_chr16_10044778_10047306_9907142_9911562_RF_1 | 94051 | 346.3 | 1.772 |
| 35 | ORF1_chr9_71446407_71452706_71476978_71478030_FF_1 | 129153 | 352.3 | 1.737 |
| 36 | ORF1_chr2_37666390_37670759_37879290_37884290_RF_l | 101904 | 369.6 | 1.741 |
| 37 | ORF1_chr5_50708409_50716050_50726245_50731870_FR_1 | 130047 | 382.9 | 1.775 |
| 38 | ORF1_chr5_122311763_122314967_122350193_122356927_FF_1 | 133785 | 385.8 | 1.812 |
| 39 | ORF1_chr4_85761047_85763139_85952092_85960583_FR_1 | 73396 | 388.6 | 1.79 |
| 40 | ORF1_chr15_44713197_44715317_44756695_44758842_RR_1 | 91402 | 396.4 | 1.707 |
| 41 | ORF1_chr9_71446407_71452706_71550704_71559890_FR_1 | 78244 | 399.3 | 1.7 |
| 42 | ORF1_chrX_45661991_45664592_45739643_45748032_RF_1 | 120043 | 405.2 | 1.736 |
| 43 | ORF1_chr4_73629979_73638181_73831449_73839428_FF_1 | 59006 | 404.4 | 1.749 |
| 44 | ORF1_chr18_68711389_68714330_68797123_68798463_RF_1 | 50615 | 422.1 | 1.705 |
| 45 | ORF1_chr14_65076556_65085228_65194791_65198794_FR_1 | 109799 | 441.4 | 1.674 |
| 46 | ORF1_chr14_53106017_53108105_53223270_53229326_RF_1 | 104855 | 437 | 1.713 |
| 47 | ORF1_chr9_127166323_127173810_127226000_127228604_RF_1 | 65446 | 434.9 | 1.734 |
| 48 | ORF1_chr1_98050628_98053768_98164665_98171570_RR_1 | 25472 | 439.8 | 1.728 |
| 49 | ORF1_chr4_153219867_153224675_153242666_153249857_FR_1 | 55153 | 444.5 | 1.865 |
| 50 | ORF1_chr2_233064861_233067168_233261787_233264668_RR_1 | 91626 | 433.6 | 1.828 |

TABLE 2.a2

| | | | | Probe sequence |
|---|---|---|---|---|
| | pfp | P.value | Type | 60 mer |
| 1 | 5.687E−07 | 4.03E−12 | SSc | CAATCAAAATGTTGCAGTATCTTTTTAATCGAGATTTAACCACATCTGATTTCATGGGTT |
| 2 | 0.000004995 | 2.123E−10 | SSc | CCAAATACTTTCAAAATATAAGTATTATTCGAGTCTTCACAACAAAAGATAGAGGACCCA |
| 3 | 0.00000558 | 1.582E−10 | SSc | TTCCACAAAGTTTATTTTTTGTTTCATTTCGAGGCTACAGTGGAGCCTCGGCTTCCCAAA |
| 4 | 0.000005773 | 2.045E−10 | SSc | TAAGACAGTGATTCCATTCGTAATAGTATCGAGTTGTTATTTATAATTCGGAACTATTCT |
| 5 | 0.000006857 | 1.457E−10 | SSc | AGTTCAATGGACAAATAAATGAAATAAATCGACATACTCCAAAACATTGAAATATTGATG |
| 6 | 0.000007637 | 1.082E−10 | SSc | CATTGGAGGCACTCAAAAAAAAAAAAAATCGATGATCTCTTCAAAGACATAACCCTTTGG |
| 7 | 0.000008788 | 4.359E−10 | SSc | CACCTTGGTTATAATGTATTTTTAAATTTCGAGTTCTTATGATGCTAAGAGAGGCTGATT |
| 8 | 0.00002258 | 1.44E−09 | SSc | ATAAATATAAGTTGAATATTTATTTTGTTCGAAGGATATGAATTCCAGCTATGGCTCTGC |
| 9 | 0.00002307 | 1.635E−09 | SSc | TTTTCAAAACAATTTTAGGATAAGCTTGTCGAGGTTATCCCTATTGGTTGAAGGTGGGAA |
| 10 | 0.00002343 | 1.992E−09 | SSc | TTAGTCTTAATTTATAAAGCAACTTACTTCGATGTTATTGCAGCTTAAAGGTCAGGCTTA |
| 11 | 0.00002535 | 1.437E−09 | SSc | TATATCAGTTTGTTTATCCATTTATCCATCGAATCAAAATATTGTTTCTACTTCTTTGGA |
| 12 | 0.00002554 | 1.99E−09 | SSc | CCTAGTTAGCTTATTTTCTCTAGGATTTTCGATATGTTATTTAATCATCAACTAATTACA |
| 13 | 0.00003265 | 3.47E−09 | SSc | AGAATTAAATCTTAAGTCTTTATGGAGATCGACTTTGAAACTAATTGTGTTTTTACCAAA |
| 14 | 0.00003301 | 4.211E−09 | SSc | TTCTTGAAGGACATACTTTAATATTTCTTCGACACTGTCTCCAACTTACTTTATCTGATT |
| 15 | 0.00003313 | 3.286E−09 | SSc | AGCTAAAGTAGATATGCTTCTTGAGAAATCGACACAAGGGTTTGTTAAAAAAAAAAGAAA |
| 16 | 0.00003443 | 5.367E−09 | SSc | ATAGCAAGTTGCATTTAGTTTTTAGCTATCGATTTTGTTTAAACCTAAAAAACAAGAAAA |
| 17 | 0.00003446 | 4.151E−09 | SSc | CAAAAAAAAAAAAAAAAAAAAAAGGTAGTCGAAAAATAAAATGATAAATAAAATAAAATA |
| 18 | 0.00003545 | 5.275E−09 | SSc | GGTTTTCTTTGTTTTGTTTAAAATCATTTCGAATCTTTAATTCACATTGAATATGTGTAC |
| 19 | 0.00003559 | 5.043E−09 | SSc | GCAAATGCAAGAGTGCAACTACCGTATTTCGAAATTAATTAGAAGAATATCTGCATTTTT |
| 20 | 0.0000365 | 4.914E−09 | SSc | AGCTGCTATCATGATTACTATATTATTATCGAACAGATTAGATATTTTAACACCAATTCT |
| 21 | 0.00003869 | 6.306E−09 | SSc | TATTTATGACAGTTTAAAAAATTCCTTGTCGAGAAATCTGCTACCTTGATTAGTAAGACT |
| 22 | 0.00003915 | 6.657E−09 | SSc | GCAGATAAAATAATACTCCAATAACTGATCGAAAGTAAAAATAAAAAGAATTACGATGCG |
| 23 | 0.00005949 | 1.222E−08 | SSc | AAAGATGAATAAAATTTGGTTCCTCCCTTCGACCCTGTGCGGACATTCCGCATACTGCCA |
| 24 | 0.00005968 | 1.142E−08 | SSc | GAGGTTTTCAAAAAGAATTATTTAAAAGTCGATTAACCATTTCTGCCATGTATGAAGGAT |
| 25 | 0.00006062 | 1.074E−08 | SSc | TATCCACCTACCAAATTATCACCCATCCTCGAAAATCTTAGCTTTGTAAAGAACTGTTAA |
| 26 | 0.00006065 | 1.203E−08 | SSc | GTTTAAAGGACAATATGTCAATAGATTCTCGAAACTATTTTGTTTGTAACTAATAAGTGA |
| 27 | 0.00006162 | 1.135E−08 | SSc | GATGGATAGATAAATGGAAAGATAGAGATCGAGTTGTGTTAATTATTAGGAAAATAACCT |
| 28 | 0.00006576 | 1.398E−08 | SSc | TATCCACCTACCAAATTATCACCCATCCTCGAAATGTTCCTGGATAATAAACACACAATT |
| 29 | 0.00007459 | 1.638E−08 | SSc | AAAGTAATAATGTAAAACTATCTTTTCCTCGATATTTGTAGGTGGTTGAAGATTAATGTT |
| 30 | 0.00008535 | 1.935E−08 | SSc | ACTTTAGATAATCTGTATATGTTTTGCATCGAAGAATATAATGGTATATGCCATGTTAAA |
| 31 | 0.00008899 | 2.081E−08 | SSc | ACCTCATTCAAAAATCATCTTCTTGATATCGAACAGTAAAAGGACTTCCTCTTTTTTTGG |
| 32 | 0.0001431 | 3.448E−08 | SSc | ATTTACTTATTTATTCATGGAATGTTTATCGAGGCTGATTTAGACAGGACTTTGTGTGCA |
| 33 | 0.0001549 | 3.842E−08 | SSc | GCCAGATATTTGGTGCGCAGATATTTGGTCGATAAATGTTTATTGCCCTTTACTGTGTGA |
| 34 | 0.0001833 | 4.676E−08 | SSc | TTTGTCATGAGTATAAAATGAGAATTCATCGAATCTGATTCCTCTTTTAGACTGTAAGTT |
| 35 | 0.0001909 | 5.005E−08 | SSc | CAAGTTCTCCCAACTAATAATTGGAAGTTCGAAATTCCAAATATGGAAACACATACTGAA |
| 36 | 0.0002251 | 6.062E−08 | SSc | ATTCCACTGTAACAAGTAAAGAAAATATTCGAGGTACACTTTAGGCGGTACCCTGTTACC |
| 37 | 0.0002526 | 6.979E−08 | SSc | GCAAGAATCCAAAATTTACTGTGCTTTATCGACTATACAATAACAGTGAATTTATGGTGT |
| 38 | 0.0002538 | 7.192E−08 | SSc | CCCATGGAAAATTAAATGATACTAAACATCGATTTAATAAGGTCAGACAGGAGCTAGGTA |
| 39 | 0.0002549 | 7.405E−08 | SSc | ATATTTTATAGTTGCAGGAAGCTACTATTCGACACTCCAGAGAGCTCTTTCTATACATAG |
| 40 | 0.0002691 | 8.007E−08 | SSc | ATTTTCTCATGATCAAAACATTCTGCTTTCGATAGTTCAGTCACCAGGTGACTGTCGTGG |
| 41 | 0.0002705 | 8.242E−08 | SSc | CAAGTTCTCCCAACTAATAATTGGAAGTTCGAATGGCTTTATTCTAAAATATATAAATTA |
| 42 | 0.000274 | 8.735E−08 | SSc | AATGAAACAGTTAAGAGGAATGCTATTTTCGACCTTCTATAGTTCTCTCATTTTCAGTAT |
| 43 | 0.0002782 | 8.673E−08 | SSc | AACAGTCAAAAAACAATAGATATTGGTATCGATGTTCTAGGAGAGCTCTTAATACAGTTA |
| 44 | 0.0003151 | 1.027E−07 | SSc | TATACTAAGCAAAACTCAGGTAAGAATTTCGATTTTCTTTAAACCTAAAAAACAAGAAAA |
| 45 | 0.0003391 | 1.225E−07 | SSc | ACCTGTTTAACTTTTTAATTGGTTCTACTCGATAAAGAATCACAAATCTCCATCTTGCGA |
| 46 | 0.0003392 | 1.178E−07 | SSc | TATTTCACTTAGCATAATATCTTCAAGGTCGACTTTTTGCCTACTTTACCTTTTATTATC |
| 47 | 0.0003396 | 1.155E−07 | SSc | TTTATGAAATATAGATGCATACACTTTGTCGAGCATCAAACATGTACTAGATCCTTTTAT |
| 48 | 0.0003408 | 1.207E−07 | SSc | TTACAAGTGGAATAGAATTTATTGGCTATCGAATTAAATTTATAATTATTTTGTTTTAGA |
| 49 | 0.0003416 | 1.259E−07 | SSc | TGCCTTTATTTTCCTGGAAAATTCTATGTCGACAGAACTACTGAAATTGTAAAGAAACTC |
| 50 | 0.000343 | 1.142E−07 | SSc | AATTAACAGTGTCTATTTTACTGAAGCTTCGAACTATAAATCCACAGTTCTAGTTGTCTT |

TABLE 2.a3

| | Probe Location | | | | | 4 kb Sequence Location | | | | |
|---|---|---|---|---|---|---|---|---|---|---|
| | Chr | Start1 | End1 | Start2 | End2 | Chr | Start1 | End1 | Start2 | End2 |
| 1 | chr15 | 94292715 | 94292744 | 94340210 | 94340239 | chr15 | 94288745 | 94292744 | 94340210 | 94344209 |
| 2 | chr4 | 102315518 | 102315547 | 102386634 | 102386663 | chr4 | 102311548 | 102315547 | 102386634 | 102390633 |
| 3 | chr8 | 38716164 | 38716193 | 38738343 | 38738372 | chr8 | 38716164 | 38720163 | 38738343 | 38742342 |
| 4 | chr5 | 132458509 | 132458538 | 132521320 | 132521349 | chr5 | 132458509 | 132462508 | 132517350 | 132521349 |
| 5 | chr12 | 96463991 | 96464020 | 96618280 | 96618309 | chr12 | 96463991 | 96467990 | 96618280 | 96622279 |
| 6 | chr11 | 79041479 | 79041508 | 79271257 | 79271286 | chr11 | 79037509 | 79041508 | 79267287 | 79271286 |
| 7 | chr1 | 11226039 | 11226068 | 11272814 | 11272843 | chr1 | 11226039 | 11230038 | 11272814 | 11276813 |
| 8 | chr2 | 159285163 | 159285192 | 159326594 | 159326623 | chr2 | 159281193 | 159285192 | 159326594 | 159330593 |
| 9 | chr8 | 90011442 | 90011471 | 90148441 | 90148470 | chr8 | 90007472 | 90011471 | 90148441 | 90152440 |
| 10 | chr3 | 113947065 | 113947094 | 113975287 | 113975316 | chr3 | 113947065 | 113951064 | 113975287 | 113979286 |
| 11 | chr3 | 149821040 | 149821069 | 150005761 | 150005790 | chr3 | 149817070 | 149821069 | 150005761 | 150009760 |
| 12 | chr5 | 60604038 | 60604067 | 60738058 | 60738087 | chr5 | 60600068 | 60604067 | 60738058 | 60742057 |
| 13 | chr6 | 53534189 | 53534218 | 53651091 | 53651120 | chr6 | 53534189 | 53538188 | 53647121 | 53651120 |
| 14 | chr5 | 88274732 | 88274761 | 88365194 | 88365223 | chr5 | 88270762 | 88274761 | 88365194 | 88369193 |
| 15 | chr9 | 76572021 | 76572050 | 76771021 | 76771050 | chr9 | 76572021 | 76576020 | 76767051 | 76771050 |
| 16 | chr18 | 68711391 | 68711420 | 68918919 | 68918948 | chr18 | 68711391 | 68715390 | 68914949 | 68918948 |
| 17 | chr5 | 134464511 | 134464540 | 134674134 | 134674163 | chr5 | 134464511 | 134468510 | 134674134 | 134678133 |
| 18 | chr8 | 92469390 | 92469419 | 92510936 | 92510965 | chr8 | 92469390 | 92473389 | 92510936 | 92514935 |
| 19 | chr10 | 54060340 | 54060369 | 54213285 | 54213314 | chr10 | 54056370 | 54060369 | 54213285 | 54217284 |
| 20 | chr1 | 109159475 | 109159504 | 109321121 | 109321150 | chr1 | 109159475 | 109163474 | 109317151 | 109321150 |
| 21 | chr12 | 9624843 | 9624872 | 9733197 | 9733226 | chr12 | 9620873 | 9624872 | 9729227 | 9733226 |
| 22 | chr14 | 89417654 | 89417683 | 89641752 | 89641781 | chr14 | 89417654 | 89421653 | 89637782 | 89641781 |
| 23 | chr1 | 155209891 | 155209920 | 155288577 | 155288606 | chr1 | 155205921 | 155209920 | 155288577 | 155292576 |
| 24 | chr5 | 173450238 | 173450267 | 173485404 | 173485433 | chr5 | 173450238 | 173454237 | 173481434 | 173485433 |
| 25 | chr3 | 43252799 | 43252828 | 43297214 | 43297243 | chr3 | 43248829 | 43252828 | 43297214 | 43301213 |
| 26 | chr2 | 118090942 | 118090971 | 118214617 | 118214646 | chr2 | 118090942 | 118094941 | 118214617 | 118218616 |
| 27 | chr5 | 96799183 | 96799212 | 96815917 | 96815946 | chr5 | 96799183 | 96803182 | 96811947 | 96815946 |
| 28 | chr3 | 43094300 | 43094329 | 43252799 | 43252828 | chr3 | 43094300 | 43098299 | 43248829 | 43252828 |
| 29 | chr9 | 16577661 | 16577690 | 16729302 | 16729331 | chr9 | 16573691 | 16577690 | 16725332 | 16729331 |
| 30 | chr3 | 107414956 | 107414985 | 107527610 | 107527639 | chr3 | 107414956 | 107418955 | 107523640 | 107527639 |
| 31 | chr12 | 13095079 | 13095108 | 13190442 | 13190471 | chr12 | 13091109 | 13095108 | 13190442 | 13194441 |
| 32 | chr5 | 35271026 | 35271055 | 35319376 | 35319405 | chr5 | 35271026 | 35275025 | 35319376 | 35323375 |
| 33 | chr13 | 109649215 | 109649244 | 109745464 | 109745493 | chr13 | 109649215 | 109653214 | 109745464 | 109749463 |
| 34 | chr16 | 10044780 | 10044809 | 9911531 | 9911560 | chr16 | 10044780 | 10048779 | 9907561 | 9911560 |
| 35 | chr9 | 71452675 | 71452704 | 71477999 | 71478028 | chr9 | 71448705 | 71452704 | 71474029 | 71478028 |
| 36 | chr2 | 37666392 | 37666421 | 37884259 | 37884288 | chr2 | 37666392 | 37670391 | 37880289 | 37884288 |
| 37 | chr5 | 50716019 | 50716048 | 50726247 | 50726276 | chr5 | 50712049 | 50716048 | 50726247 | 50730246 |
| 38 | chr5 | 122314936 | 122314965 | 122356896 | 122356925 | chr5 | 122310966 | 122314965 | 122352926 | 122356925 |
| 39 | chr4 | 85763108 | 85763137 | 85952094 | 85952123 | chr4 | 85759138 | 85763137 | 85952094 | 85956093 |
| 40 | chr15 | 44713199 | 44713228 | 44756697 | 44756726 | chr15 | 44713199 | 44717198 | 44756697 | 44760696 |
| 41 | chr9 | 71452675 | 71452704 | 71550706 | 71550735 | chr9 | 71448705 | 71452704 | 71550706 | 71554705 |
| 42 | chrX | 45661993 | 45662022 | 45748001 | 45748030 | chrX | 45661993 | 45665992 | 45744031 | 45748030 |
| 43 | chr4 | 73638150 | 73638179 | 73839397 | 73839426 | chr4 | 73634180 | 73638179 | 73835427 | 73839426 |
| 44 | chr18 | 68711391 | 68711420 | 68798432 | 68798461 | chr18 | 68711391 | 68715390 | 68794462 | 68798461 |
| 45 | chr14 | 65085197 | 65085226 | 65194793 | 65194822 | chr14 | 65081227 | 65085226 | 65194793 | 65198792 |
| 46 | chr14 | 53106019 | 53106048 | 53229295 | 53229324 | chr14 | 53106019 | 53110018 | 53225325 | 53229324 |
| 47 | chr9 | 127166325 | 127166354 | 127228573 | 127228602 | chr9 | 127166325 | 127170324 | 127224603 | 127228602 |
| 48 | chr1 | 98050630 | 98050659 | 98164667 | 98164696 | chr1 | 98050630 | 98054629 | 98164667 | 98168666 |
| 49 | chr4 | 153224644 | 153224673 | 153242668 | 153242697 | chr4 | 153220674 | 153224673 | 153242668 | 153246667 |
| 50 | chr2 | 233064863 | 233064892 | 233261789 | 233261818 | chr2 | 233064863 | 233068862 | 233261789 | 233265788 |

TABLE 2.a4

| | probe | Primer ID |
|---|---|---|
| 1 | ORF1_chr15_94287688_94292746_94340208_94346941_FR_1 | OBD168-401 |
| 2 | ORF1_chr4_102312731_102315549_102386632_102391817_FR_1 | OBD168-405 |
| 3 | ORF1_chr8_38716162_38718294_38738341_38742581_RR_1 | OBD168-409 |
| 4 | ORF1_chr5_132458507_132464714_132517598_132521351_RF_1 | OBD168-413 |
| 5 | ORF1_chr12_96463989_96466636_96618278_96624162_RR_1 | OBD168-417 |
| 6 | ORF1_chr11_79036088_79041510_79269569_79271288_FF_1 | OBD168-421 |
| 7 | ORF1_chr1_11226037_11228251_11272812_11274518_RR_1 | OBD168-425 |
| 8 | ORF1_chr2_159283360_159285194_159326592_159328925_FR_1 | OBD168-429 |
| 9 | ORF1_chr8_90002099_90011473_90148439_90150313_FR_1 | OBD168-433 |
| 10 | ORF1_chr3_113947063_113952443_113975285_113978229_RR_1 | OBD168-437 |
| 11 | ORF1_chr3_149818709_149821071_150005759_150009319_FR_1 | OBD168-441 |
| 12 | ORF1_chr5_60597760_60604069_60738056_60741292_FR_1 | OBD168-445 |
| 13 | ORF1_chr6_53534187_53536920_53639583_53651122_RF_1 | OBD168-449 |
| 14 | ORF1_chr5_88269888_88274763_88365192_88376199_FR_1 | OBD168-453 |
| 15 | ORF1_chr9_76572019_76577216_76769644_76771052_RF_1 | OBD168-457 |
| 16 | ORF1_chr18_68711389_68714330_68911644_68918950_RF_1 | OBD168-461 |
| 17 | ORF1_chr5_134464509_134469933_134674132_134676030_RR_1 | OBD168-465 |
| 18 | ORF1_chr8_92469388_92478361_92510934_92515600_RR_1 | OBD168-469 |
| 19 | ORF1_chr10_54058576_54060371_54213283_54215754_FR_1 | OBD168-473 |
| 20 | ORF1_chr1_109159473_109163477_109318126_109321152_RF_1 | OBD168-477 |
| 21 | ORF1_chr12_9614159_9624874_9729793_9733228_FF_1 | OBD168-481 |
| 22 | ORF1_chr14_89417652_89420597_89636182_89641783_RF_1 | OBD168-485 |
| 23 | ORF1_chr1_155206612_155209922_155288575_155290398_FR_1 | OBD168-489 |
| 24 | ORF1_chr5_173450236_173452725_173479240_173485435_RF_1 | OBD168-493 |
| 25 | ORF1_chr3_43246875_43252830_43297212_43303273_FR_1 | OBD168-497 |
| 26 | ORF1_chr2_118090940_118096647_118214615_118218048_RR_1 | OBD168-501 |
| 27 | ORF1_chr5_96799181_96801649_96813450_96815948_RF_1 | OBD168-505 |
| 28 | ORF1_chr3_43094298_43105527_43246875_43252830_RF_1 | OBD168-509 |
| 29 | ORF1_chr9_16574863_16577692_16721839_16729333_FF_1 | OBD168-513 |
| 30 | ORF1_chr3_107414954_107426087_107525934_107527641_RF_1 | OBD168-517 |
| 31 | ORF1_chr12_13090807_13095110_13190440_13193042_FR_1 | OBD168-521 |
| 32 | ORF1_chr5_35271024_35276749_35319374_35321203_RR_1 | OBD168-525 |
| 33 | ORF1_chr13_109649213_109651298_109745462_109746722_RR_1 | OBD168-529 |
| 34 | ORF1_chr16_10044778_10047306_9907142_9911562_RF_1 | OBD168-533 |
| 35 | ORF1_chr9_71446407_71452706_71476978_71478030_FF_1 | OBD168-537 |
| 36 | ORF1_chr2_37666390_37670759_37879290_37884290_RF_1 | OBD168-541 |
| 37 | ORF1_chr5_50708409_50716050_50726245_50731870_FR_1 | OBD168-545 |
| 38 | ORF1_chr5_122311763_122314967_122350193_122356927_FF_1 | OBD168-549 |
| 39 | ORF1_chr4_85761047_85763139_85952092_85960583_FR_1 | OBD168-553 |
| 40 | ORF1_chr15_44713197_44715317_44756695_44758842_RR_1 | OBD168-557 |
| 41 | ORF1_chr9_71446407_71452706_71550704_71559890_FR_1 | OBD168-561 |
| 42 | ORF1_chrX_45661991_45664592_45739643_45748032_RF_1 | OBD168-565 |
| 43 | ORF1_chr4_73629979_73638181_73831449_73839428_FF_1 | OBD168-569 |
| 44 | ORF1_chr18_68711389_68714330_68797123_68798463_RF_1 | OBD168-573 |
| 45 | ORF1_chr14_65076556_65085228_65194791_65198794_FR_1 | OBD168-577 |
| 46 | ORF1_chr14_53106017_53108105_53223270_53229326_RF_1 | OBD168-581 |
| 47 | ORF1_chr9_127166323_127173810_127226000_127228604_RF_1 | OBD168-585 |
| 48 | ORF1_chr1_98050628_98053768_98164665_98171570_RR_1 | OBD168-589 |
| 49 | ORF1_chr4_153219867_153224675_153242666_153249857_FR_1 | OBD168-593 |
| 50 | ORF1_chr2_233064861_233067168_233261787_233264668_RR_1 | OBD168-597 |

TABLE 2.a5

| | Primer Sequence | Primer ID | Primer Sequence |
|---|---|---|---|
| 1 | GCTTGTATTGGAGACTAAACATAG | OBD168-403 | CCTCTAAAACTCATCAATAACAAT |
| 2 | TCTCTTATCTCTTCATTGGTAACTGC | OBD168-407 | TAGTCCTCAGGTTTAGGCAAGTTG |
| 3 | ACTGTTCTGTTGATTGGTTGGTGATA | OBD168-411 | AGGGACACAGAAGCCAAATGAGAGAG |
| 4 | CATCAAGTTTTCAGAATAGTAAATC | OBD168-415 | CACCTGGGAACATTATTTGGCATA |
| 5 | CCTGGGCAACAGAGTGAGATTCT | OBD168-419 | CCTTTCCTGGTCCCTCCTCATCA |
| 6 | CAGGACCATTGAGAGAATAACAGATA | OBD168-423 | GTTCCTGAGCCGCCCGATAAAGC |
| 7 | CACCTTGGTTATAGGACATC | OBD168-427 | GAGTTCTTATGATGCTAAGA |
| 8 | TGCTCATTTCTGTATCCACACCAT | OBD168-431 | TATGTGGCTTGTCATCCTTATTTT |
| 9 | GGCTATGTCCCCTAAGGAAACACACA | OBD168-435 | CACCAGGTTGACATCTTTTCAGTGGG |
| 10 | GAAACTGCCCTGAGATGAGAGTCCTT | OBD168-439 | GCTGTGGATGAAAGGAAGGAGAGACA |
| 11 | TAAGCAGTCATTCCCTGATGCCCCTA | OBD168-443 | CACCTGAGCAATGCGGATAAAGCAGA |
| 12 | ATGCCTCAACCATTACATCTTTTCCT | OBD168-447 | AGTAGACAGATGGGTGGGATTCTTCC |
| 13 | GAGGAAGAGGAATGTCCGCTAACACA | OBD168-451 | GCCAGCACCCAAGATTTAGCCAAGTT |
| 14 | TTCTATTGTGTTTCGTGGTTCCTGGA | OBD168-455 | ATACCTGAGTAGGAGTTAGCGTCTA |
| 15 | TCAACTATCAATGCGAAAGTAAAT | OBD168-459 | CACATTTCCCATCTCATTATTATTA |
| 16 | TTGGATTCATAGCAGTTCTAAAAT | OBD168-463 | TATCAATGTATTATCTCTGCCTAT |
| 17 | GGCAACAGAGCGAGACTCCTTCT | OBD168-467 | AGGCTGGGATTACAGGCGTAGGC |
| 18 | TATCCTTCCAGTCTACCTTCTTTT | OBD168-471 | GTTCTGTGGTCTCCTATCTACATA |
| 19 | CTGACTGACTATGATTGTTTGTAG | OBD168-475 | ATTTGGCATTTCACTTTTGGTTTA |
| 20 | TGGGCTCTGGAGTTGGGATGCCA | OBD168-479 | TGTTGGTTGTGTGCCAGCAGCCA |
| 21 | CAAGTCTGCTATTGAACCCCTCT | OBD168-483 | ATTTCCTTCTGCTCCCCAAGTGG |
| 22 | GCTGAGAGAGTCAAGGGAAAACCC | OBD168-487 | CGTGGAATCTCCTCTTCTCACCCC |
| 23 | GACCAAACGACTCACAGCCAGGC | OBD168-491 | CTTGGCAGTGGAGACATACAGCG |
| 24 | CCCAAGCAGGGCAACCATCAAGG | OBD168-495 | ACAATCAGTGGGCTGGATGTGGC |
| 25 | AAAATGTCTGTCCCCACCATCCCAGG | OBD168-499 | TGACTTCTCATCAGGTCCGAGTGTA |
| 26 | CAGAGGAGTGGGTTATTTTCAAGGGA | OBD168-503 | TTGATTTGCCCTCAGTAACCTCGCTG |
| 27 | ATGTATGTGTGGATAGACGGATGGAT | OBD168-507 | CCCTTTGGACGGGAATACCTCTCACT |
| 28 | CCCACCATCCCAGGCAATCCCAA | OBD168-511 | GGAAATCACTGGGTCTGTTGCCC |
| 29 | AAAATCAGGAGTTTGGAGAAAATA | OBD168-515 | TCATAAAAGTAGTCAGCAGGAAAA |
| 30 | GAGACCTCATCTGTGGTGTTTCCTGG | OBD168-519 | CTTGTTGAAGTGAGGGATTTTGTCCT |
| 31 | TCCACCAGGGAGAGACACAAGGA | OBD168-523 | TCCCCTGGATTGTGTGCTGGGCA |
| 32 | TAACCCCAGCCTAAGACAAAATAG | OBD168-527 | CCATTTTGTTTGGTTTTGCTTTGC |
| 33 | GGAAGGGAAGATACAGAGGGTTTCT | OBD168-531 | AAACAAGCGGCGGCAGGACCCCT |
| 34 | TAAAATGGGCATAATCATAGCAGCCA | OBD168-535 | GGTCATTGATTAGGGAATAGTAACAC |
| 35 | GAGAGGGAGGGTGAGAGAGAACCT | OBD168-539 | GGGCTTGGTGTTTCCTTGGTTCC |
| 36 | CCATCCAAAAGAGTCATACACAGGAA | OBD168-543 | GTTTGCTTAGTCCTTACTGCTTGCCT |
| 37 | GCTTCTGTCTCCTTCTTTTCAGTCTC | OBD168-547 | TAGATTACACATACATCCTCCATTGG |
| 38 | GCATTTACTTTGTGCTTATTTGTA | OBD168-551 | TTACTCTAAACAAGCCACTGAAGT |
| 39 | CTTTTGGACTATTGATTTTCCTAT | OBD168-555 | AAAGCACAGGCAGATGTATGAATA |
| 40 | GAGGAGGAAAATAGACCTTCAGAGGC | OBD168-559 | AAGACCAGACACATTTCAACGCCACG |
| 41 | GAGGGAGGGTGAGAGAGAACCTATCT | OBD168-563 | GGAGTATGATTGTCCATCTTCTGCCT |
| 42 | TACTTCTGCGTGCTTTATTCCAGGCA | OBD168-567 | CCAGAGGATGACTTGATTTGGAGTGG |
| 43 | CAACATCACTAATCATCAGAGATA | OBD168-571 | TTGGTGTTCTCATAGCATCTTCTA |
| 44 | GCTGGGAAGCAGAAGGATTTGGAGTT | OBD168-575 | TCAGTCCTCTGCTTTTATCTTTGAGA |
| 45 | GGAGGCAGGTTTCTTTTCACATCCAC | OBD168-579 | TGTTATTTACGAAGAGTGTTGTGAGC |
| 46 | CGTCATTCTACTTTCTGTCTCCAT | OBD168-583 | GTAATAAGTGAGAGTCATACAATA |
| 47 | TTTCTTTGTTTCATAGTGAGGTGA | OBD168-587 | GTAATACAGAAATAGTAACACCTA |
| 48 | TCAGTGTTCAAATGGATGACAAAA | OBD168-591 | AGAACTACATCTGAAAATCAAAGT |
| 49 | GTTTGTTTAGATGACTGTCTCTTATCAG | OBD168-595 | CTTTACAGGCAGACCCAGGGAGC |
| 50 | GGGCACATACCCCTTCCTGGTCT | OBD168-599 | GTGCCTAAAGCCACAGGAGTGAG |

TABLE 2.a6

| | probe | Marker |
|---|---|---|
| 1 | ORF1_chr15_94287688_94292746_94340208_94346941_FR_1 | OBD168-401.403 |
| 2 | ORF1_chr4_102312731_102315549_102386632_102391817_FR_1 | OBD168-405.407 |
| 3 | ORF1_chr8_38716162_38718294_38738341_38742581_RR_1 | OBD168-409.411 |
| 4 | ORF1_chr5_132458507_132464714_132517598_132521351_RF_1 | OBD168-413.415 |
| 5 | ORF1_chr12_96463989_96466636_96618278_96624162_RR_1 | OBD168-417.419 |
| 6 | ORF1_chr11_79036088_79041510_79269569_79271288_FF_1 | OBD168-421.423 |
| 7 | ORF1_chr1_11226037_11228251_11272812_11274518_RR_1 | OBD168-425.427 |
| 8 | ORF1_chr2_159283360_159285194_159326592_159328925_FR_1 | OBD168-429.431 |
| 9 | ORF1_chr8_90002099_90011473_90148439_90150313_FR_1 | OBD168-433.435 |
| 10 | ORF1_chr3_113947063_113952443_113975285_113978229_RR_1 | OBD168-437.439 |
| 11 | ORF1_chr3_149818709_149821071_150005759_150009319_FR_1 | OBD168-441.443 |
| 12 | ORF1_chr5_60597760_60604069_60738056_60741292_FR_1 | OBD168-445.447 |
| 13 | ORF1_chr6_53534187_53536920_53639583_53651122_RF_1 | OBD168-449.451 |
| 14 | ORF1_chr5_88269888_88274763_88365192_88376199_FR_1 | OBD168-453.455 |
| 15 | ORF1_chr9_76572019_76577216_76769644_76771052_RF_1 | OBD168-457.459 |
| 16 | ORF1_chr18_68711389_68714330_68911644_68918950_RF_1 | OBD168-461.463 |
| 17 | ORF1_chr5_134464509_134469933_134674132_134676030_RR_1 | OBD168-465.467 |
| 18 | ORF1_chr8_92469388_92478361_92510934_92515600_RR_1 | OBD168-469.471 |
| 19 | ORF1_chr10_54058576_54060371_54213283_54215754_FR_1 | OBD168-473.475 |
| 20 | ORF1_chr1_109159473_109163477_109318126_109321152_RF_1 | OBD168-477.479 |
| 21 | ORF1_chr12_9614159_9624874_9729793_9733228_FF_1 | OBD168-481.483 |
| 22 | ORF1_chr14_89417652_89420597_89636182_89641783_RF_1 | OBD168-485.487 |
| 23 | ORF1_chr1_155206612_155209922_155288575_155290398_FR_1 | OBD168-489.491 |
| 24 | ORF1_chr5_173450236_173452725_173479240_173485435_RF_1 | OBD168-493.495 |
| 25 | ORF1_chr3_43246875_43252830_43297212_43303273_FR_1 | OBD168-497.499 |
| 26 | ORF1_chr2_118090940_118096647_118214615_118218048_RR_1 | OBD168-501.503 |
| 27 | ORF1_chr5_96799181_96801649_96813450_96815948_RF_1 | OBD168-505.507 |
| 28 | ORF1_chr3_43094298_43105527_43246875_43252830_RF_1 | OBD168-509.511 |
| 29 | ORF1_chr9_16574863_16577692_16721839_16729333_FF_1 | OBD168-513.515 |
| 30 | ORF1_chr3_107414954_107426087_107525934_107527641_RF_1 | OBD168-517.519 |
| 31 | ORF1_chr12_13090807_13095110_13190440_13193042_FR_1 | OBD168-521.523 |
| 32 | ORF1_chr5_35271024_35276749_35319374_35321203_RR_1 | OBD168-525.527 |
| 33 | ORF1_chr13_109649213_109651298_109745462_109746722_RR_1 | OBD168-529.531 |
| 34 | ORF1_chr16_10044778_10047306_9907142_9911562_RF_1 | OBD168-533.535 |
| 35 | ORF1_chr9_71446407_71452706_71476978_71478030_FF_1 | OBD168-537.539 |
| 36 | ORF1_chr2_37666390_37670759_37879290_37884290_RF_1 | OBD168-541.543 |
| 37 | ORF1_chr5_50708409_50716050_50726245_50731870_FR_1 | OBD168-545.547 |
| 38 | ORF1_chr5_122311763_122314967_122350193_122356927_FF_1 | OBD168-549.551 |
| 39 | ORF1_chr4_85761047_85763139_85952092_85960583_FR_1 | OBD168-553.555 |
| 40 | ORF1_chr15_44713197_44715317_44756695_44758842_RR_1 | OBD168-557.559 |
| 41 | ORF1_chr9_71446407_71452706_71550704_71559890_FR_1 | OBD168-561.563 |
| 42 | ORF1_chrX_45661991_45664592_45739643_45748032_RF_1 | OBD168-565.567 |
| 43 | ORF1_chr4_73629979_73638181_73831449_73839428_FF_1 | OBD168-569.571 |
| 44 | ORF1_chr18_68711389_68714330_68797123_68798463_RF_1 | OBD168-573.575 |
| 45 | ORF1_chr14_65076556_65085228_65194791_65198794_FR_1 | OBD168-577.579 |
| 46 | ORF1_chr14_53106017_53108105_53223270_53229326_RF_1 | OBD168-581.583 |
| 47 | ORF1_chr9_127166323_127173810_127226000_127228604_RF_1 | OBD168-585.587 |
| 48 | ORF1_chr1_98050628_98053768_98164665_98171570_RR_1 | OBD168-589.591 |
| 49 | ORF1_chr4_153219867_153224675_153242666_153249857_FR_1 | OBD168-593.595 |
| 50 | ORF1_chr2_233064861_233067168_233261787_233264668_RR_1 | OBD168-597.599 |

TABLE 2.b1

| | | | | |
|---|---|---|---|---|
| 51 | ORF1_chr18_62330039_62332469_62410773_62412005_FR_1 | 65192 | 239.5 | −1.875 |
| 52 | ORF1_chr1_97723527_97730826_97845524_97850752_RF_1 | 50784 | 228.9 | −1.885 |
| 53 | ORF1_chr5_165127546_165128761_165171328_165175416_FR_1 | 19637 | 216.9 | −2.379 |
| 54 | ORF1_chr2_26077134_26081726_26188355_26191067_RF_1 | 69487 | 178.6 | −1.995 |
| 55 | ORF1_chr5_165127546_165128761_165223844_165225058_FF_1 | 64468 | 215.5 | −2.376 |
| 56 | ORF1_chr18_62240628_62243651_62330039_62332469_RF_1 | 133666 | 346.6 | −1.762 |
| 57 | ORF1_chr18_35525412_35527471_35609926_35611497_FF_1 | 82154 | 326.1 | −1.904 |
| 58 | ORF1_chr10_93741396_93744278_93870079_93871714_FR_1 | 18832 | 373.7 | −1.721 |
| 59 | ORF1_chr2_186622411_186639782_186682119_186696186_RR_1 | 60297 | 398.7 | −2.055 |
| 60 | ORF1_chr12_52217955_52219158_52307864_52312912_RR_1 | 94808 | 645.1 | −1.637 |
| 61 | ORF1_chr4_168499900_168503445_168627683_168631715_RR_1 | 19017 | 638.6 | −1.749 |
| 62 | ORF1_chr19_56561558_56564720_56759775_56765600_RF_1 | 104682 | 688.2 | −1.605 |
| 63 | ORF1_chr18_62330039_62332469_62555999_62562221_FR_1 | 50516 | 672 | −1.631 |
| 64 | ORF1_chr20_44492930_44498158_44629984_44632053_RF_1 | 124521 | 685.8 | −1.717 |
| 65 | ORF1_chr6_15591030_15592334_15658715_15661455_FR_1 | 337 | 714.7 | −1.592 |
| 66 | ORF1_chr15_69599001_69607474_69822688_69824653_FF_1 | 62666 | 745.6 | −1.585 |
| 67 | ORF1_chr3_194783856_194786305_194878644_194884153_RR_1 | 17829 | 770.7 | −1.677 |
| 68 | ORF1_chr2_29059303_29063818_29079780_29082596_FR_1 | 40095 | 763.6 | −1.742 |
| 69 | ORF1_chr1_97845524_97850752_97969457_97975300_FR_1 | 50177 | 785.2 | −1.6 |
| 70 | ORF1_chr10_93674996_93683570_93741396_93744278_RF_1 | 47914 | 862.8 | −1.578 |
| 71 | ORF1_chr8_125289724_125291437_125304189_125311876_RR_1 | 102207 | 885.4 | −1.562 |
| 72 | ORF1_chr5_77084813_77086498_77232748_77239462_RR_1 | 51608 | 913 | −1.636 |
| 73 | ORF1_chr17_68654144_68657529_68785810_68796098_FR_1 | 41397 | 984.6 | −1.724 |
| 74 | ORF1_chr1_112396884_112399115_112478496_112485740_RR_1 | 98481 | 1020 | −1.678 |
| 75 | ORF1_chr1_207314651_207319651_207566031_207567951_RR_1 | 51534 | 1088 | −1.97 |
| 76 | ORF1_chr3_194783856_194786305_194980518_194987236_RR_1 | 16693 | 1145 | −1.542 |
| 77 | ORF1_chr4_24971631_24973362_25125726_25129421_RR_1 | 21906 | 1155 | −1.541 |
| 78 | ORF1_chr1_117925651_117930301_117977839_117981106_FF_1 | 60395 | 1120 | −1.555 |
| 79 | ORF1_chr18_62330039_62332469_62459933_62463601_FF_1 | 79455 | 1142 | −1.521 |
| 80 | ORF1_chr1_16764579_16768424_17044824_17050263_RR_1 | 35783 | 1173 | −1.58 |
| 81 | ORF1_chr3_42701255_42712462_42763077_42767575_RR_1 | 96660 | 1137 | −1.56 |
| 82 | ORF1_chr2_200711316_200714252_200861419_200863026_RR_1 | 37379 | 1120 | −1.47 |
| 83 | ORF1_chr10_119537441_119541197_119587019_119594844_RR_1 | 102190 | 1191 | −1.523 |
| 84 | ORF1_chr8_57997832_57999789_58156560_58160085_FF_1 | 97562 | 1274 | −1.49 |
| 85 | ORF1_chr5_54687073_54701690_54895820_54899919_RF_1 | 84406 | 1298 | −1.495 |
| 86 | ORF1_chr22_37276476_37281194_37296506_37298053_FF_1 | 89194 | 1268 | −1.533 |
| 87 | ORF1_chr9_27616981_27624076_27731177_27739077_RR_1 | 19905 | 1332 | −1.487 |
| 88 | ORF1_chr13_57155089_57159703_57172839_57181375_RR_1 | 118355 | 1298 | −1.539 |
| 89 | ORF1_chr2_127388555_127395831_127512403_127514497_RR_1 | 125959 | 1266 | −1.633 |
| 90 | ORF1_chr14_61568474_61572943_61720413_61722720_FR_1 | 105496 | 1233 | −1.987 |
| 91 | ORF1_chr11_104153980_104160882_104179897_104181773_FF_1 | 18635 | 1291 | −1.85 |
| 92 | ORF1_chr4_15897457_15905803_15999719_16002207_RR_1 | 19816 | 1244 | −1.501 |
| 93 | ORF1_chr17_17631398_17633309_17824119_17825835_RR_1 | 73392 | 1264 | −1.608 |
| 94 | ORF1_chr17_18194373_18201018_18300185_18301934_FF_1 | 43770 | 1320 | −1.552 |
| 95 | ORF1_chr5_169694390_169702047_169742878_169747823_FF_1 | 15015 | 1359 | −1.527 |
| 96 | ORF1_chr1_224094766_224099331_224328317_224334418_FF_1 | 46944 | 1354 | −1.479 |
| 97 | ORF1_chr19_51484258_51487562_51764536_51772179_RR_1 | 27427 | 1262 | −1.555 |
| 98 | ORF1_chr9_92152672_92155419_92403506_92407832_FR_1 | 104212 | 1388 | −1.947 |
| 99 | ORF1_chr1_117925651_117930301_118024887_118028035_FR_1 | 17564 | 1467 | −1.49 |
| 100 | ORF1_chr16_87246411_87247863_87484414_87491902_RF_1 | 65474 | 1403 | −1.601 |

TABLE 2.b2

| | | | | |
|---|---|---|---|---|
| 51 | 0.0002952 | 1.046E−08 | pHC | GAGAATCAATTCCATTTTTAAAGCTTAGTCGAGTCCCTTACAGAAAATGATGTAGTATTT |
| 52 | 0.0003058 | 8.667E−09 | pHC | GTCACCTATTTTTTCCAACTTAATTTGATCGATTCATTACACTTTATATTAAGTAGTTTT |
| 53 | 0.0003268 | 6.947E−09 | pHC | TTTGTAACAATAGCAAAAGTTCAGTTCTTCGAGCTAATAGAAGGATGGTGAAGTAAATTT |
| 54 | 0.0004359 | 3.089E−09 | pHC | TACCTTCCAACAGGAAGTGCAAACTAATTCGAAATAATTCAGACATGATTGTGTTTTACT |
| 55 | 0.0004767 | 6.755E−09 | pHC | TTTGTAACAATAGCAAAAGTTCAGTTCTTCGATAAACATTTATTCAAAGGCATGAATGAC |
| 56 | 0.0008269 | 4.687E−08 | pHC | GAGAATCAATTCCATTTTTAAAGCTTAGTCGAATGCTAATCCTCTTCTATTAATGAATTC |
| 57 | 0.0008633 | 3.67E−08 | pHC | GTCACAGAGATCATGGTAAAGAGAAAATTCGATTACATAAATATTTCTCTAAGGTTGCTG |
| 58 | 0.0009938 | 6.338E−08 | pHC | AGAAATGAACAACACATTGTAATATACATCGAAAAGAACTAATAATGCTAAGGATTCTGA |
| 59 | 0.001157 | 8.195E−08 | pHC | AGAAGACTCACCAAAATTTTATCCTGTTTCGAACCTTATAATGGTGATAAATCATTAATG |
| 60 | 0.005398 | 5.355E−07 | pHC | AAATCAAGACGGAGGAACTATTTCAGATTCGAGGTAGTGGCAGCTAATAGCTCTTAAGGG |
| 61 | 0.005591 | 0.000000515 | pHC | TTGAAGTCTGTTTGTGAAATACTTTGAGTCGAAAGACAAACGGCAAAATGAGAAAACAAT |
| 62 | 0.005695 | 6.859E−07 | pHC | CGCGTGATGTGTTTGAAGGTGTGTTTGTTCGAAAAAGACTTGCATTTATGCAGAGTGAGG |
| 63 | 0.005892 | 6.262E−07 | pHC | GAGAATCAATTCCATTTTTAAAGCTTAGTCGATTCTTGCAAATGAATTTTGTTAAATGTT |
| 64 | 0.00597 | 6.768E−07 | pHC | AAATTATTTTGCTTTCTATTTCTCCTCTTCGATCTTTCATATTTCATCCTGGTTTCACAA |
| 65 | 0.006212 | 7.923E−07 | pHC | AGTTTTCTCAGTTATTGAAATGAATCTCTCGAAGTATGTACTTTGGTATGTCCAAAAGTC |
| 66 | 0.00691 | 9.303E−07 | pHC | AATGAAGTCCCAGGCTTTCGTCTCACGTTCGAATAACAACCAGTTTAAAAACATACTAAA |
| 67 | 0.007091 | 0.000001055 | pHC | TTTCTAAATTTCCAAGGTTCTTTCCAGTTCGACCAATGAAACTGATTAAGACCATACAAG |
| 68 | 0.007188 | 0.000001019 | pHC | GCATGAACAACTGTTTTTCACTTTGATTTCGAATTTCTTGACTTTTGACTGGTTGTTTAT |
| 69 | 0.007265 | 0.000001132 | pHC | GTCACCTATTTTTTCCAACTTAATTTGATCGATTTCTGCATTTCCTTGAACTGAATCTTA |
| 70 | 0.009917 | 0.000001616 | pHC | AGAAATGAACAACACATTGTAATATACATCGAAACTGTCACTACATTTTACCTAATTTAG |
| 71 | 0.01048 | 0.000001781 | pHC | CTCAAGAAAGAAAACCTAAGCTTTAAATTCGACTCTAATCTATAAAACTAAGATACTTAA |
| 72 | 0.01128 | 0.000001998 | pHC | CAACTGAGGAACTGGAACTTCATTAGAGTCGAAGTGCTCTGTGGTGATGCAGCAGGTTGA |
| 73 | 0.01439 | 0.00000265 | pHC | AGAAGCTGTATGATTTGTAGTAATTCATTCGAATAGTCAGTAGCTGCCTTTGCAAAGATG |
| 74 | 0.01577 | 0.000003017 | pHC | CCAAAACAGTCCCAAAACAGGAGAGCTCTCGATGGGACTATTGGAAGATTTCTCAGAAGA |
| 75 | 0.01867 | 0.000003836 | pHC | TGGTGCATAATGAAGACTAAAGCGTTCCTCGATTTAAAAATAGTTTATTTTAATAATGTT |
| 76 | 0.01927 | 0.000004641 | pHC | TTTCTAAATTTCCAAGGTTCTTTCCAGTTCGAGGCAATTCTAAAGAACAATAAGAGATTA |
| 77 | 0.01932 | 0.00000479 | pHC | GGTGAGTGGCAGATAGCTGGGAAAACATTCGAGCCAAGCTGAGTGACAGCTTTGTCACGG |
| 78 | 0.01947 | 0.000004276 | pHC | GCAAGTTGTATGGGAGGTAGTTTAAATATCGAAGTATCTCAAATAACATTTTTGAGAATG |
| 79 | 0.01965 | 0.000004594 | pHC | GAGAATCAATTCCATTTTTAAAGCTTAGTCGATTAGGATGATGGCAATGGAAATAACAAG |
| 80 | 0.01985 | 0.000005064 | pHC | AGCTCAGGCAATCCATGCTTGGAGAAAGTCGATAGATGGCAAATTTCTTGATACGGGGAG |
| 81 | 0.01997 | 0.000004527 | pHC | TTGTGAGGAGCTAAGGGAGACTCACTGATCGAAAAAGATTTATGAACAGAAAAAGGAAAG |
| 82 | 0.02009 | 0.00000427 | pHC | CAACAAGAACGACTTTCTTTTTTCTTCTTCGACTATGATAATCTTTCTCATTTATAGACC |
| 83 | 0.02046 | 0.000005365 | pHC | ACTAGCTTGCAAGGATTGTTGCAAAGTTTCGATGAAAGCACAAATAAATAATTAAAGGCA |
| 84 | 0.02204 | 0.000006872 | pHC | AGCGAATTTAAGGCAGTAATAAGTGCAGTCGAGGCAGTGGGAGTTGAATCCTCTCCCCTC |
| 85 | 0.0221 | 0.000007358 | pHC | ATTAGGAGTGGGGGCCTTGAGAGGTAATTCGATCTTTTCAAAGAACCACATTTTAGTTTT |
| 86 | 0.02217 | 0.000006755 | pHC | GAGCTGCAAGGCCTTTTAAAGCTCAGCCTCGATTTGTATTCCACAGAGCTTATCATTCTC |
| 87 | 0.02237 | 0.000008085 | pHC | AATACTACTTTGCATATATAATAAAGTTTCGAGAATGGACTAATAAACACATGTTTCTGA |
| 88 | 0.02256 | 0.000007354 | pHC | TCAGTCTAGAACATAACAGAAAATGTCTTCGACTCTTTTTACAAAAGGCCACTTGACATC |
| 89 | 0.02258 | 0.000006718 | pHC | AATGTACACAACCAGTCATAACCAAGTATCGAAAAGATTTTTACAGAGTTTTCCCCCTTC |
| 90 | 0.0226 | 0.000006085 | pHC | GGCAGATGGATCACTTGAGGTCAAGAGTTCGAGATGCAGCCAGATCTCGGCGAAGTAAAG |
| 91 | 0.02263 | 0.000007217 | pHC | CTTATAATGGGTAGAAGCAGACTATCATTCGATAAGAAAAAGAGAGATATCCAAATAAAA |
| 92 | 0.02278 | 0.000006294 | pHC | TTGGTTAGAGATAAAAACCCTGTAAATGTCGACCAGTGAAATAACTAAAATACTGTGACA |
| 93 | 0.02297 | 0.000006673 | pHC | AGGCTGATATTTGTTCTTTAGCTGTGTGTCGAATGGTAGAAGGGAGATAATATGGGTGCA |
| 94 | 0.023 | 0.000007821 | pHC | GCTTACTTGGTCATTAGATCCACAGAGTTCGACTCTTTAACCACACGGCTTGCCTAGTTC |
| 95 | 0.02313 | 0.000008685 | pHC | GAGGATGAAAATTAAATGACTTTTAATGTCGAGAATTAAACTCAGTTTAAGAGACTGCTA |
| 96 | 0.02328 | 0.000008577 | pHC | GGCAGGTGGATTGCTTGAGCTCAGGAGTTCGAAAAAGAAAAGAAAAGAACATAACAGAAC |
| 97 | 0.0234 | 0.000006633 | pHC | GTAGTCCAGGGGAAGAGAAAAATGTGAATCGATAATTGTGAAAGTCACTTCCGCTCTCTG |
| 98 | 0.02457 | 0.0000094 | pHC | GTACAGTTCCAACGAGCGGAGTGGTGGGTCGAAGCAATATTTAAAAGCTTAGAGGATGTT |
| 99 | 0.02494 | 0.00001149 | pHC | GCAAGTTGTATGGGAGGTAGTTTAAATATCGAAATCTTCTGATCTATTATTGATGACATG |
| 100 | 0.02507 | 0.00000977 | pHC | CTGCAGCCCGTAGTTGTGGATGATGGAGTCGATCGTGCAGACAGGAGACAAACAGCAAGA |

TABLE 2.b3

| | | | | | | | | | | |
|---|---|---|---|---|---|---|---|---|---|---|
| 51 | chr18 | 62332438 | 62332467 | 62410775 | 62410804 | chr18 | 62328468 | 62332467 | 62410775 | 62414774 |
| 52 | chr1 | 97723529 | 97723558 | 97850721 | 97850750 | chr1 | 97723529 | 97727528 | 97846751 | 97850750 |
| 53 | chr5 | 165128730 | 165128759 | 165171330 | 165171359 | chr5 | 165124760 | 165128759 | 165171330 | 165175329 |
| 54 | chr2 | 26077136 | 26077165 | 26191036 | 26191065 | chr2 | 26077136 | 26081135 | 26187066 | 26191065 |
| 55 | chr5 | 165128730 | 165128759 | 165225027 | 165225056 | chr5 | 165124760 | 165128759 | 165221057 | 165225056 |
| 56 | chr18 | 62240630 | 62240659 | 62332438 | 62332467 | chr18 | 62240630 | 62244629 | 62328468 | 62332467 |
| 57 | chr18 | 35527440 | 35527469 | 35611466 | 35611495 | chr18 | 35523470 | 35527469 | 35607496 | 35611495 |
| 58 | chr10 | 93744247 | 93744276 | 93870081 | 93870110 | chr10 | 93740277 | 93744276 | 93870081 | 93874080 |
| 59 | chr2 | 186622413 | 186622442 | 186682121 | 186682150 | chr2 | 186622413 | 186626412 | 186682121 | 186686120 |
| 60 | chr12 | 52217957 | 52217986 | 52307866 | 52307895 | chr12 | 52217957 | 52221956 | 52307866 | 52311865 |
| 61 | chr4 | 168499902 | 168499931 | 168627685 | 168627714 | chr4 | 168499902 | 168503901 | 168627685 | 168631684 |
| 62 | chr19 | 56561560 | 56561589 | 56765569 | 56765598 | chr19 | 56561560 | 56565559 | 56761599 | 56765598 |
| 63 | chr18 | 62332438 | 62332467 | 62556001 | 62556030 | chr18 | 62328468 | 62332467 | 62556001 | 62560000 |
| 64 | chr20 | 44492932 | 44492961 | 44632022 | 44632051 | chr20 | 44492932 | 44496931 | 44628052 | 44632051 |
| 65 | chr6 | 15592303 | 15592332 | 15658717 | 15658746 | chr6 | 15588333 | 15592332 | 15658717 | 15662716 |
| 66 | chr15 | 69607443 | 69607472 | 69824622 | 69824651 | chr15 | 69603473 | 69607472 | 69820652 | 69824651 |
| 67 | chr3 | 194783858 | 194783887 | 194878646 | 194878675 | chr3 | 194783858 | 194787857 | 194878646 | 194882645 |
| 68 | chr2 | 29063787 | 29063816 | 29079782 | 29079811 | chr2 | 29059817 | 29063816 | 29079782 | 29083781 |
| 69 | chr1 | 97850721 | 97850750 | 97969459 | 97969488 | chr1 | 97846751 | 97850750 | 97969459 | 97973458 |
| 70 | chr10 | 93674998 | 93675027 | 93744247 | 93744276 | chr10 | 93674998 | 93678997 | 93740277 | 93744276 |
| 71 | chr8 | 125289726 | 125289755 | 125304191 | 125304220 | chr8 | 125289726 | 125293725 | 125304191 | 125308190 |
| 72 | chr5 | 77084815 | 77084844 | 77232750 | 77232779 | chr5 | 77084815 | 77088814 | 77232750 | 77236749 |
| 73 | chr17 | 68657498 | 68657527 | 68785812 | 68785841 | chr17 | 68653528 | 68657527 | 68785812 | 68789811 |
| 74 | chr1 | 112396886 | 112396915 | 112478498 | 112478527 | chr1 | 112396886 | 112400885 | 112478498 | 112482497 |
| 75 | chr1 | 207314653 | 207314682 | 207566033 | 207566062 | chr1 | 207314653 | 207318652 | 207566033 | 207570032 |
| 76 | chr3 | 194783858 | 194783887 | 194980520 | 194980549 | chr3 | 194783858 | 194787857 | 194980520 | 194984519 |
| 77 | chr4 | 24971633 | 24971662 | 25125728 | 25125757 | chr4 | 24971633 | 24975632 | 25125728 | 25129727 |
| 78 | chr1 | 117930270 | 117930299 | 117981075 | 117981104 | chr1 | 117926300 | 117930299 | 117977105 | 117981104 |
| 79 | chr18 | 62332438 | 62332467 | 62463570 | 62463599 | chr18 | 62328468 | 62332467 | 62459600 | 62463599 |
| 80 | chr1 | 16764581 | 16764610 | 17044826 | 17044855 | chr1 | 16764581 | 16768580 | 17044826 | 17048825 |
| 81 | chr3 | 42701257 | 42701286 | 42763079 | 42763108 | chr3 | 42701257 | 42705256 | 42763079 | 42767078 |
| 82 | chr2 | 200711318 | 200711347 | 200861421 | 200861450 | chr2 | 200711318 | 200715317 | 200861421 | 200865420 |
| 83 | chr10 | 119537443 | 119537472 | 119587021 | 119587050 | chr10 | 119537443 | 119541442 | 119587021 | 119591020 |
| 84 | chr8 | 57999758 | 57999787 | 58160054 | 58160083 | chr8 | 57995788 | 57999787 | 58156084 | 58160083 |
| 85 | chr5 | 54687075 | 54687104 | 54899888 | 54899917 | chr5 | 54687075 | 54691074 | 54895918 | 54899917 |
| 86 | chr22 | 37281163 | 37281192 | 37298022 | 37298051 | chr22 | 37277193 | 37281192 | 37294052 | 37298051 |
| 87 | chr9 | 27616983 | 27617012 | 27731179 | 27731208 | chr9 | 27616983 | 27620982 | 27731179 | 27735178 |
| 88 | chr13 | 57155091 | 57155120 | 57172841 | 57172870 | chr13 | 57155091 | 57159090 | 57172841 | 57176840 |
| 89 | chr2 | 127388557 | 127388586 | 127512405 | 127512434 | chr2 | 127388557 | 127392556 | 127512405 | 127516404 |
| 90 | chr14 | 61572912 | 61572941 | 61720415 | 61720444 | chr14 | 61568942 | 61572941 | 61720415 | 61724414 |
| 91 | chr11 | 104160851 | 104160880 | 104181742 | 104181771 | chr11 | 104156881 | 104160880 | 104177772 | 104181771 |
| 92 | chr4 | 15897459 | 15897488 | 15999721 | 15999750 | chr4 | 15897459 | 15901458 | 15999721 | 16003720 |
| 93 | chr17 | 17631400 | 17631429 | 17824121 | 17824150 | chr17 | 17631400 | 17635399 | 17824121 | 17828120 |
| 94 | chr17 | 18200987 | 18201016 | 18301903 | 18301932 | chr17 | 18197017 | 18201016 | 18297933 | 18301932 |
| 95 | chr5 | 169702016 | 169702045 | 169747792 | 169747821 | chr5 | 169698046 | 169702045 | 169743822 | 169747821 |
| 96 | chr1 | 224099300 | 224099329 | 224334387 | 224334416 | chr1 | 224095330 | 224099329 | 224330417 | 224334416 |
| 97 | chr19 | 51484260 | 51484289 | 51764538 | 51764567 | chr19 | 51484260 | 51488259 | 51764538 | 51768537 |
| 98 | chr9 | 92155388 | 92155417 | 92403508 | 92403537 | chr9 | 92151418 | 92155417 | 92403508 | 92407507 |
| 99 | chr1 | 117930270 | 117930299 | 118024889 | 118024918 | chr1 | 117926300 | 117930299 | 118024889 | 118028888 |
| 100 | chr16 | 87246413 | 87246442 | 87491871 | 87491900 | chr16 | 87246413 | 87250412 | 87487901 | 87491900 |

TABLE 2.b4

| | | |
|---|---|---|
| 51 | ORF1_chr18_62330039_62332469_62410773_62412005_FR_1 | OBD168-601 |
| 52 | ORF1_chr1_97723527_97730826_97845524_97850752_RF_1 | OBD168-605 |
| 53 | ORF1_chr5_165127546_165128761_165171328_165175416_FR_1 | OBD168-609 |
| 54 | ORF1_chr2_26077134_26081726_26188355_26191067_RF_1 | OBD168-613 |
| 55 | ORF1_chr5_165127546_165128761_165223844_165225058_FF_1 | OBD168-617 |
| 56 | ORF1_chr18_62240628_62243651_62330039_62332469_RF_1 | OBD168-621 |
| 57 | ORF1_chr18_35525412_35527471_35609926_35611497_FF_1 | OBD168-625 |
| 58 | ORF1_chr10_93741396_93744278_93870079_93871714_FR_1 | OBD168-629 |
| 59 | ORF1_chr2_186622411_186639782_186682119_186696186_RR_1 | OBD168-633 |
| 60 | ORF1_chr12_52217955_52219158_52307864_52312912_RR_1 | OBD168-637 |
| 61 | ORF1_chr4_168499900_168503445_168627683_168631715_RR_1 | OBD168-641 |
| 62 | ORF1_chr19_56561558_56564720_56759775_56765600_RF_1 | OBD168-645 |
| 63 | ORF1_chr18_62330039_62332469_62555999_62562221_FR_1 | OBD168-649 |
| 64 | ORF1_chr20_44492930_44498158_44629984_44632053_RF_1 | OBD168-653 |
| 65 | ORF1_chr6_15591030_15592334_15658715_15661455_FR_1 | OBD168-657 |
| 66 | ORF1_chr15_69599001_69607474_69822688_69824653_FF_1 | OBD168-661 |
| 67 | ORF1_chr3_194783856_194786305_194878644_194884153_RR_1 | OBD168-665 |
| 68 | ORF1_chr2_29059303_29063818_29079780_29082596_FR_1 | OBD168-669 |
| 69 | ORF1_chr1_97845524_97850752_97969457_97975300_FR_1 | OBD168-673 |
| 70 | ORF1_chr10_93674996_93683570_93741396_93744278_RF_1 | OBD168-677 |
| 71 | ORF1_chr8_125289724_125291437_125304189_125311876_RR_1 | OBD168-681 |
| 72 | ORF1_chr5_77084813_77086498_77232748_77239462_RR_1 | OBD168-685 |
| 73 | ORF1_chr17_68654144_68657529_68785810_68796098_FR_1 | OBD168-689 |
| 74 | ORF1_chr1_112396884_112399115_112478496_112485740_RR_1 | OBD168-693 |
| 75 | ORF1_chr1_207314651_207319651_207566031_207567951_RR_1 | OBD168-697 |
| 76 | ORF1_chr3_194783856_194786305_194980518_194987236_RR_1 | OBD168-701 |
| 77 | ORF1_chr4_24971631_24973362_25125726_25129421_RR_1 | OBD168-705 |
| 78 | ORF1_chr1_117925651_117930301_117977839_117981106_FF_1 | OBD168-709 |
| 79 | ORF1_chr18_62330039_62332469_62459933_62463601_FF_1 | OBD168-713 |
| 80 | ORF1_chr1_16764579_16768424_17044824_17050263_RR_1 | OBD168-717 |
| 81 | ORF1_chr3_42701255_42712462_42763077_42767575_RR_1 | OBD168-721 |
| 82 | ORF1_chr2_200711316_200714252_200861419_200863026_RR_1 | OBD168-725 |
| 83 | ORF1_chr10_119537441_119541197_119587019_119594844_RR_1 | OBD168-729 |
| 84 | ORF1_chr8_57997832_57999789_58156560_58160085_FF_1 | OBD168-733 |
| 85 | ORF1_chr5_54687073_54701690_54895820_54899919_RF_1 | OBD168-737 |
| 86 | ORF1_chr22_37276476_37281194_37296506_37298053_FF_1 | OBD168-741 |
| 87 | ORF1_chr9_27616981_27624076_27731177_27739077_RR_1 | OBD168-745 |
| 88 | ORF1_chr13_57155089_57159703_57172839_57181375_RR_1 | OBD168-749 |
| 89 | ORF1_chr2_127388555_127395831_127512403_127514497_RR_1 | OBD168-753 |
| 90 | ORF1_chr14_61568474_61572943_61720413_61722720_FR_1 | OBD168-757 |
| 91 | ORF1_chr11_104153980_104160882_104179897_104181773_FF_1 | OBD168-761 |
| 92 | ORF1_chr4_15897457_15905803_15999719_16002207_RR_1 | OBD168-765 |
| 93 | ORF1_chr17_17631398_17633309_17824119_17825835_RR_1 | OBD168-769 |
| 94 | ORF1_chr17_18194373_18201018_18300185_18301934_FF_1 | OBD168-773 |
| 95 | ORF1_chr5_169694390_169702047_169742878_169747823_FF_1 | OBD168-777 |
| 96 | ORF1_chr1_224094766_224099331_224328317_224334418_FF_1 | OBD168-781 |
| 97 | ORF1_chr19_51484258_51487562_51764536_51772179_RR_1 | OBD168-785 |
| 98 | ORF1_chr9_92152672_92155419_92403506_92407832_FR_1 | OBD168-789 |
| 99 | ORF1_chr1_117925651_117930301_118024887_118028035_FR_1 | OBD168-793 |
| 100 | ORF1_chr16_87246411_87247863_87484414_87491902_RF_1 | OBD168-797 |

TABLE 2.b5

| | | | |
|---|---|---|---|
| 51 | GCCCCTGTCTTCTCTCAACTTCATAG | OBD168-603 | CCGAATGGATGGTTGTGTCTGTGTTG |
| 52 | TCAGCCTCAGAATGCCAGAGTGACTG | OBD168-607 | CGAAGACTCTGACAAACACAGTGGTA |
| 53 | GCTGCTATGAGGCATTGTAGAGGAGA | OBD168-611 | GAACAGCAGACTTGTGGACCTTATCA |
| 54 | AACAGGCAGAGAGGTGGCTTCAGATG | OBD168-615 | GAGAAGCAACACCACAAAAGCAGGAA |
| 55 | GCTGCTATGAGGCATTGTAGAGGAGA | OBD168-619 | GCTCTCTGTCCATTCCTCAGTTTCTC |
| 56 | CCCTGTCTTCTCTCAACTTCATAGTC | OBD168-623 | TAGCAGGAACTATGTGCCACTGTGCC |
| 57 | CCCACTGCTTGGCTTTGGAGGAA | OBD168-627 | GCAGAGACTTTCCACCCTGTGCC |
| 58 | GCCCCTTTCTGGGAACAACAACG | OBD168-631 | GGAGCAAGTAGGGAAGCAGTGGTC |
| 59 | ACCCTGTGCCCTTGAGCATACTGAAT | OBD168-635 | TGCCTTGTTTCCCCTTTGCCTTTAGC |
| 60 | ACCTCGGAGAAGCCTGGCAGACA | OBD168-639 | GTGCTTAGTGCCTGGCTCATAGC |
| 61 | AGAAAGTGGCAGACTCAGGGTGGAAG | OBD168-643 | CCCTTTGCGTTAGGAGTTTCAGATTG |
| 62 | GCGAATGTCCCTACAAGTGTGGG | OBD168-647 | ACTGGAAGCAAAGGTGCCTGGGC |
| 63 | GCCCCTGTCTTCTCTCAACTTCATAG | OBD168-651 | CTGATACTGTGTAGGGAGTCTGATGA |
| 64 | TACGGCTCCTCCATCTACCCAGC | OBD168-655 | CTCCTAAGATAAGTGACTCCTTGTG |
| 65 | CGGTAAATCTAAAGGACAATGCTGGT | OBD168-659 | GCCACAGACTGAAGAGGATTGGTTCT |
| 66 | CCGTTGCTCAAGAACCATCCAGAGTC | OBD168-663 | GCAAACTTCAGGCTGTATTGTTCTCG |
| 67 | GGTAGATTCCAACTCTGATTCCATTT | OBD168-667 | CTTTCCTTCTCTCTTCCTTATTGAGC |
| 68 | GCTCATTGGCAGAAGCAGGTTTT | OBD168-671 | TGGCAAAGCAACACCCTAAGGAT |
| 69 | TCAGCCTCAGAATGCCAGAGTGACTG | OBD168-675 | TGAGAAACCACAGCCACAGCAGAAAT |
| 70 | CTTTACCCTCAGTTTCCTTATCGGGA | OBD168-679 | GCAGAGAGGGAGATGTTGATGGAGGT |
| 71 | TAACAACTGCTAACTGGAGACACCA | OBD168-683 | ACTTGCTGTCTCGTTTCCTTGCCCC |
| 72 | GCAACACTACTCTCTCTCCTTCCTTG | OBD168-687 | GCAGTATTCCCTTTACAGTTATTGGT |
| 73 | GAGGCAGGCTTTTCGTTCCAGCA | OBD168-691 | GAGACATCTTCCCCACTTGCTCGC |
| 74 | TACAAAGGGTGAAGGAGAGGAGGTCC | OBD168-695 | CCCTATTTTCTCCTGGTCAGCAAGTT |
| 75 | CACCTTCCATTGATACTCTCACTTTG | OBD168-699 | GCACAAGACCAAAGGATTTATCGCAC |
| 76 | CCACGCCTCAGCCAATCACTCCAAAA | OBD168-703 | GAAAGCCATTGTCCCAACACCAAGTA |
| 77 | CAGTAGTTCTAAAAGAGAGGATGGTA | OBD168-707 | GCCAGTAAACCAAGGCTTATTAGGAT |
| 78 | GGTTTGGTCGTGAACTGTCAGAAAGG | OBD168-711 | CTTTGTATTTCTTTCCTTCAGTCTGG |
| 79 | GGGTTCGCATCACATCCCACAGA | OBD168-715 | CCCACTCACAGTTTGCTGACCCC |
| 80 | TTATCCCCTGTCTGACTTGTTCCCCG | OBD168-719 | GCGTTACATCTGTTGTGCCAGCAAAA |
| 81 | GGAGTGGGAGTCAGAGAAAGGGTCTT | OBD168-723 | CCTCACTTGTGCTTTCTGTTCATCAC |
| 82 | CTGAAGCAGACTCTCACTGGAATACA | OBD168-727 | ACATAGCAGTGCCCAGGAGAACAAGC |
| 83 | GCTCATCCACTTCCTTGCTTGCTTCT | OBD168-731 | GATGGCAGGAGTTTATGGAAAGGGCT |
| 84 | AACATCAGTCAGGAAAGACAAGGTTC | OBD168-735 | TGGTGTGTCCTGGGTTTTGTGGAGCA |
| 85 | CTTGTTATGGACCAAATGTCTGGC | OBD168-739 | CAGCAGAAGGAAAGAAATAATAAAA |
| 86 | GTCCTCCGTCTTCCAACAGGCTA | OBD168-743 | AGGCACCATCCTAAGCCCTGGGT |
| 87 | GTGTTATTTGTATCTCCTCCTGGC | OBD168-747 | ATTATGTGCTTACCCAAAAGAAAT |
| 88 | TCAGACATCTCAAAAGCAGCAGTTC | OBD168-751 | CTTTTGAACTGCCAAGTGAGGATGT |
| 89 | GGAGATGAGGAGCGAAAGAAAGGGTT | OBD168-755 | TGTGGTAGGACAGATAGGGAAGAAGG |
| 90 | GCCTCAGGGAACGCTTCTTAGGG | OBD168-759 | GGAAGTGGCAACTGATGAGCAAGC |
| 91 | AGAATACCCTATGATGGAGAGTTCCG | OBD168-763 | CCACCTCCCCTACTTTCCCATAACAT |
| 92 | CAGATGAGGGCAGTCTTGGGAGA | OBD168-767 | CTCTCAGTCCAACTCCCACCGAC |
| 93 | GAGATAAAGGTGTGAGCCACTGT | OBD168-771 | CATCCCAGACCATCAGCCCCGCT |
| 94 | GGAGTGGAGGACTTGATACACCG | OBD168-775 | TGGGAGTGGTTTGGGAGGTGTGC |
| 95 | TCTATTCTGCTCCTACACAGTGAAAT | OBD168-779 | GCAAACTTGTATCCAGTGAAGGC |
| 96 | GTAATCCCAGCACTTTGGTAGGC | OBD168-783 | GAGGCTTCCTGCTTGCTGGGAGC |
| 97 | GCCTGGGCAACAGAGCGAGACTC | OBD168-787 | ATTACAATACCCTTGCTTTGGGACAA |
| 98 | GTATTCCAAGGCAGTCAATCCTGGGA | OBD168-791 | ATTAGTATGTCTGCTGAAGGCACTTA |
| 99 | TGAACTGTCAGAAAGGAGGCGAAAGC | OBD168-795 | GGAATGACACCTTCAAATGACAAATG |
| 100 | CAGGAACTCATCGCCCGTGCGGC | OBD168-799 | TGAGACCATTCCAAAGCAGAGGG |

TABLE 2.b6

| | | |
|---|---|---|
| 51 | ORF1_chr18_62330039_62332469_62410773_62412005_FR_1 | OBD168-601.603 |
| 52 | ORF1_chr1_97723527_97730826_97845524_97850752_RF_1 | OBD168-605.607 |
| 53 | ORF1_chr5_165127546_165128761_165171328_165175416_FR_1 | OBD168-609.611 |
| 54 | ORF1_chr2_26077134_26081726_26188355_26191067_RF_1 | OBD168-613.615 |
| 55 | ORF1_chr5_165127546_165128761_165223844_165225058_FF_1 | OBD168-617.619 |
| 56 | ORF1_chr18_62240628_62243651_62330039_62332469_RF_1 | OBD168-621.623 |
| 57 | ORF1_chr18_35525412_35527471_35609926_35611497_FF_1 | OBD168-625.627 |
| 58 | ORF1_chr10_93741396_93744278_93870079_93871714_FR_1 | OBD168-629.631 |
| 59 | ORF1_chr2_186622411_186639782_186682119_186696186_RR_1 | OBD168-633.635 |
| 60 | ORF1_chr12_52217955_52219158_52307864_52312912_RR_1 | OBD168-637.639 |
| 61 | ORF1_chr4_168499900_168503445_168627683_168631715_RR_1 | OBD168-641.643 |
| 62 | ORF1_chr19_56561558_56564720_56759775_56765600_RF_1 | OBD168-645.647 |
| 63 | ORF1_chr18_62330039_62332469_62555999_62562221_FR_1 | OBD168-649.651 |
| 64 | ORF1_chr20_44492930_44498158_44629984_44632053_RF_1 | OBD168-653.655 |
| 65 | ORF1_chr6_15591030_15592334_15658715_15661455_FR_1 | OBD168-657.659 |
| 66 | ORF1_chr15_69599001_69607474_69822688_69824653_FF_1 | OBD168-661.663 |
| 67 | ORF1_chr3_194783856_194786305_194878644_194884153_RR_1 | OBD168-665.667 |
| 68 | ORF1_chr2_29059303_29063818_29079780_29082596_FR_1 | OBD168-669.671 |
| 69 | ORF1_chr1_97845524_97850752_97969457_97975300_FR_1 | OBD168-673.675 |
| 70 | ORF1_chr10_93674996_93683570_93741396_93744278_RF_1 | OBD168-677.679 |
| 71 | ORF1_chr8_125289724_125291437_125304189_125311876_RR_1 | OBD168-681.683 |
| 72 | ORF1_chr5_77084813_77086498_77232748_77239462_RR_1 | OBD168-685.687 |
| 73 | ORF1_chr17_68654144_68657529_68785810_68796098_FR_1 | OBD168-689.691 |
| 74 | ORF1_chr1_112396884_112399115_112478496_112485740_RR_1 | OBD168-693.695 |
| 75 | ORF1_chr1_207314651_207319651_207566031_207567951_RR_1 | OBD168-697.699 |
| 76 | ORF1_chr3_194783856_194786305_194980518_194987236_RR_1 | OBD168-701.703 |
| 77 | ORF1_chr4_24971631_24973362_25125726_25129421_RR_1 | OBD168-705.707 |
| 78 | ORF1_chr1_117925651_117930301_117977839_117981106_FF_1 | OBD168-709.711 |
| 79 | ORF1_chr18_62330039_62332469_62459933_62463601_FF_1 | OBD168-713.715 |
| 80 | ORF1_chr1_16764579_16768424_17044824_17050263_RR_1 | OBD168-717.719 |
| 81 | ORF1_chr3_42701255_42712462_42763077_42767575_RR_1 | OBD168-721.723 |
| 82 | ORF1_chr2_200711316_200714252_200861419_200863026_RR_1 | OBD168-725.727 |
| 83 | ORF1_chr10_119537441_119541197_119587019_119594844_RR_1 | OBD168-729.731 |
| 84 | ORF1_chr8_57997832_57999789_58156560_58160085_FF_1 | OBD168-733.735 |
| 85 | ORF1_chr5_54687073_54701690_54895820_54899919_RF_1 | OBD168-737.739 |
| 86 | ORF1_chr22_37276476_37281194_37296506_37298053_FF_1 | OBD168-741.743 |
| 87 | ORF1_chr9_27616981_27624076_27731177_27739077_RR_1 | OBD168-745.747 |
| 88 | ORF1_chr13_57155089_57159703_57172839_57181375_RR_1 | OBD168-749.451 |
| 89 | ORF1_chr2_127388555_127395831_127512403_127514497_RR_1 | OBD168-753.755 |
| 90 | ORF1_chr14_61568474_61572943_61720413_61722720_FR_1 | OBD168-757.759 |
| 91 | ORF1_chr11_104153980_104160882_104179897_104181773_FF_1 | OBD168-761.763 |
| 92 | ORF1_chr4_15897457_15905803_15999719_16002207_RR_1 | OBD168-765.767 |
| 93 | ORF1_chr17_17631398_17633309_17824119_17825835_RR_1 | OBD168-769.771 |
| 94 | ORF1_chr17_18194373_18201018_18300185_18301934_FF_1 | OBD168-773.775 |
| 95 | ORF1_chr5_169694390_169702047_169742878_169747823_FF_1 | OBD168-777.779 |
| 96 | ORF1_chr1_224094766_224099331_224328317_224334418_FF_1 | OBD168-781.783 |
| 97 | ORF1_chr19_51484258_51487562_51764536_51772179_RR_1 | OBD168-785.787 |
| 98 | ORF1_chr9_92152672_92155419_92403506_92407832_FR_1 | OBD168-789.791 |
| 99 | ORF1_chr1_117925651_117930301_118024887_118028035_FR_1 | OBD168-793.795 |
| 100 | ORF1_chr16_87246411_87247863_87484414_87491902_RF_1 | OBD168-797.799 |

TABLE 3.a1

| | Probe | gene.index | RP/Rsum |
|---|---|---|---|
| 1 | ORF1_8_143363265_143368758_143397073_143399812_RR | 137 | 1022.182606 |
| 2 | ORF1_X_48750795_48754365_48796124_48801821_FF | 136 | 1020.280557 |
| 3 | ORF1_4_186172696_186175767_186190828_186192765_FF | 56 | 567.5154132 |
| 4 | ORF1_1_26201425_26205480_26333363_26336159_FR | 26 | 367.3594761 |
| 5 | ORF1_2_70887659_70889000_70979229_70981565_FF | 218 | 1455.232994 |
| 6 | ORF1_2_112539682_112540702_112816387_112823836_RR | 414 | 2408.255424 |
| 7 | ORF1_12_52037813_52040902_52237944_52243119_FR | 269 | 1762.228575 |
| 8 | ORF1_11_10562525_10569392_10617125_10621846_RR | 40 | 489.4037173 |
| 9 | ORF1_2_43086424_43092123_43178166_43179822_FF | 43 | 493.6790445 |
| 10 | ORF1_17_67419703_67421985_67604580_67609081_RR | 47 | 520.8353844 |
| 11 | ORF1_19_2777060_2780381_2901738_2905384_RF | 45 | 519.5921477 |
| 12 | ORF1_17_43300561_43304860_43444308_43448547_RR | 436 | 2506.508269 |
| 13 | ORF1_15_74686122_74687597_74772372_74779791_FR | 58 | 576.6524083 |
| 14 | ORF1_10_62056179_62058893_62096285_62100217_RR | 63 | 590.6246291 |
| 15 | ORF1_15_76885704_76887654_76983002_76985852_RF | 533 | 2890.294194 |
| 16 | ORF1_11_10637008_10641060_10678778_10685244_FR | 81 | 693.4429129 |
| 17 | ORF1_3_122768838_122771609_122793452_122794847_FR | 563 | 2999.066738 |
| 18 | ORF1_5_140242642_140251155_140386390_140387430_FF | 83 | 702.8036709 |
| 19 | ORF1_2_143119651_143128757_143274179_143284059_FR | 528 | 2861.529024 |
| 20 | ORF1_7_1769382_1772142_1917082_1921849_RF | 223 | 1465.773343 |
| 21 | ORF1_13_28585301_28589104_28625877_28627714_FR | 112 | 886.7494008 |
| 22 | ORF1_11_10637008_10641060_10826892_10830503_FR | 110 | 877.5566621 |
| 23 | ORF1_17_43300561_43304860_43353578_43358798_RR | 133 | 1018.405989 |
| 24 | ORF1_18_63921019_63923125_64068334_64074049_RR | 474 | 2662.422604 |
| 25 | ORF1_20_31989698_31994926_32038678_32045816_RF | 554 | 2973.092177 |
| 26 | ORF1_11_35040602_35047084_35078579_35084103_FR | 290 | 1888.328908 |
| 27 | ORF1_18_45762052_45766373_45829537_45837293_FF | 477 | 2676.14117 |
| 28 | ORF1_22_42713609_42716051_42864112_42866876_FR | 127 | 993.9971762 |
| 29 | ORF1_21_29039171_29043861_29092829_29094361_FF | 119 | 954.7111896 |
| 30 | ORF1_X_101616906_101623004_101715574_101717864_FF | 622 | 3249.214339 |
| 31 | ORF1_16_85533210_85536278_85655930_85660237_FR | 259 | 1702.530556 |
| 32 | ORF1_16_57546345_57549019_57808053_57811713_RF | 138 | 1026.72489 |
| 33 | ORF1_20_50192390_50194162_50280026_50281924_RF | 326 | 2050.835647 |
| 34 | ORF1_2_25155910_25160743_25292182_25293890_FR | 640 | 3317.320304 |
| 35 | ORF1_7_151781262_151783710_151930364_151932111_RR | 350 | 2142.221032 |
| 36 | ORF1_2_74503923_74507137_74519716_74521847_RF | 164 | 1139.344398 |
| 37 | ORF1_6_52573576_52576507_52715481_52719514_FR | 307 | 1975.310453 |
| 38 | ORF1_10_104325690_104332059_104442837_104447074_RR | 383 | 2255.373656 |
| 39 | ORF1_11_10376885_10382511_10637008_10641060_RF | 154 | 1086.637744 |
| 40 | ORF1_19_7270757_7273737_7367544_7370291_FR | 160 | 1122.566671 |
| 41 | ORF1_6_15165975_15168155_15248586_15249747_RF | 266 | 1736.571207 |
| 42 | ORF1_1_158181667_158187628_158243613_158252050_FF | 162 | 1130.926813 |
| 43 | ORF1_11_65044650_65046952_65060361_65063993_FF | 234 | 1538.948472 |
| 44 | ORF1_6_31267448_31269252_31531115_31534994_RF | 157 | 1095.305893 |
| 45 | ORF1_17_81104538_81109597_81345804_81351809_RR | 602 | 3166.187603 |
| 46 | ORF1_3_101887192_101889071_101943010_101950739_RF | 388 | 2281.449083 |
| 47 | ORF1_4_2639345_2642002_2800083_2804902_RR | 233 | 1534.145351 |
| 48 | ORF1_9_123259504_123263022_123339568_123345302_FR | 181 | 1242.495673 |
| 49 | ORF1_3_51243406_51244925_51388915_51391864_FF | 355 | 2161.353049 |
| 50 | ORF1_8_73772858_73775783_73991731_73996964_RF | 172 | 1188.857634 |
| 51 | ORF1_7_1901148_1902503_2033188_2038434_RF | 362 | 2193.315447 |

TABLE 3.a2

| | FC: (class1/class2) | pfp | P.value | Type |
|---|---|---|---|---|
| 1 | 2.254198009 | 0.582954788 | 0.000565875 | Early SSC |
| 2 | 1.926442523 | 0.585246077 | 0.000563953 | Early SSC |
| 3 | 1.819576685 | 0.481315009 | 0.000190978 | Early SSC |
| 4 | 1.789862545 | 0.452448159 | 8.33504E−05 | Early SSC |
| 5 | 1.786660212 | 0.696259246 | 0.001075456 | Early SSC |
| 6 | 1.731351133 | 0.906180999 | 0.002658157 | Early SSC |
| 7 | 1.72782494 | 0.79699158 | 0.001519047 | Early SSC |
| 8 | 1.684693818 | 0.510408273 | 0.000144658 | Early SSC |
| 9 | 1.679467596 | 0.48264036 | 0.000147047 | Early SSC |
| 10 | 1.665042972 | 0.488329576 | 0.000162621 | Early SSC |
| 11 | 1.66364181 | 0.507750192 | 0.000161893 | Early SSC |
| 12 | 1.646347341 | 0.924029095 | 0.002854548 | Early SSC |
| 13 | 1.632056892 | 0.478789454 | 0.00019676 | Early SSC |
| 14 | 1.623263928 | 0.460927015 | 0.000205749 | Early SSC |
| 15 | 1.61294807 | 0.97372438 | 0.003677295 | Early SSC |
| 16 | 1.579010636 | 0.483049755 | 0.000277231 | Early SSC |
| 17 | 1.57167124 | 0.984233204 | 0.003926193 | Early SSC |
| 18 | 1.57069125 | 0.483257957 | 0.000284199 | Early SSC |
| 19 | 1.560692362 | 0.965656771 | 0.003612618 | Early SSC |
| 20 | 1.542279296 | 0.689591095 | 0.001089587 | Early SSC |
| 21 | 1.535126946 | 0.549667601 | 0.000436198 | Early SSC |
| 22 | 1.527885593 | 0.549057283 | 0.000427933 | Early SSC |
| 23 | 1.526693744 | 0.59643941 | 0.000562061 | Early SSC |
| 24 | 1.518323432 | 0.946326887 | 0.003178226 | Early SSC |
| 25 | 1.513503028 | 0.984923145 | 0.003866138 | Early SSC |
| 26 | 1.503017029 | 0.837079106 | 0.001720005 | Early SSC |
| 27 | 1.500622056 | 0.949008348 | 0.003207404 | Early SSC |
| 28 | 1.497953885 | 0.597522112 | 0.000537679 | Early SSC |
| 29 | 1.496480575 | 0.592329774 | 0.000499431 | Early SSC |
| 30 | 1.489761392 | 1.026554835 | 0.004524158 | Early SSC |
| 31 | 1.487826891 | 0.777966877 | 0.001427664 | Early SSC |
| 32 | 1.482472871 | 0.583437831 | 0.000570478 | Early SSC |
| 33 | 1.478605452 | 0.863503709 | 0.00199456 | Early SSC |
| 34 | 1.477688489 | 1.034924223 | 0.004693035 | Early SSC |
| 35 | 1.477264434 | 0.869597722 | 0.002156511 | Early SSC |
| 36 | 1.475249588 | 0.593584457 | 0.00068975 | Early SSC |
| 37 | 1.475130467 | 0.857292939 | 0.001864803 | Early SSC |
| 38 | 1.469690742 | 0.871292473 | 0.002364438 | Early SSC |
| 39 | 1.467937731 | 0.579847865 | 0.000632703 | Early SSC |
| 40 | 1.467830037 | 0.592200913 | 0.000671358 | Early SSC |
| 41 | 1.466878812 | 0.784983374 | 0.001479474 | Early SSC |
| 42 | 1.465675235 | 0.592850241 | 0.000680496 | Early SSC |
| 43 | 1.465342374 | 0.717660029 | 0.001189871 | Early SSC |
| 44 | 1.463447732 | 0.577069248 | 0.000641938 | Early SSC |
| 45 | 1.459551418 | 1.013212781 | 0.004321778 | Early SSC |
| 46 | 1.458144163 | 0.877911423 | 0.002413502 | Early SSC |
| 47 | 1.45177916 | 0.716682261 | 0.001183172 | Early SSC |
| 48 | 1.449946912 | 0.629677907 | 0.000807537 | Early SSC |
| 49 | 1.449756998 | 0.871099423 | 0.002191096 | Early SSC |
| 50 | 1.449681428 | 0.611542481 | 0.000745282 | Early SSC |
| 51 | 1.447185412 | 0.876984215 | 0.002249394 | Early SSC |

TABLE 3.a3

| | 60 mer |
|---|---|
| 1 | GGGTCTCCCCATATTGCCTAGGCTGGTCTCGAGGGAACTCCCTCCCCGCACCCCCAGCAT |
| 2 | CAGTCCAGTCTGAGTGACTCTAGCGATGTCGACTGCCCGCCCAGATCCCGCCTCCAGAGG |
| 3 | ACGGAGTGGAAGGAGCTGCAACTTTCCATCGAGTTCGCCCTCCCTGACAGGGAGTTGTAA |
| 4 | GGGTTTCACCATGTTGGTCAGGCTGGTCTCGAGGCCAGCCTGACCAACATGAAGAAACCC |
| 5 | CGCGGCCGTCAGAGGGCGCGGCCTACACTCGATCTTATCCTTTGACCTTGCCCACTCTTT |
| 6 | AATGCGCAGTCTTCTTTCCTTCCTTTTGTCGAACTCCTAACCTTGTGATCTACCCGCCTC |
| 7 | CTGGTCCTGCCCTCACTCTGGTGCGTAGTCGAGCCAGCATGAGCTCTCTGTTCCTGCAGA |
| 8 | ATACACAGGATAGATTTCAAGATCATTCTCGATGTACTTCTCTGCCTCAGCCTACGTGTC |
| 9 | GGATTTCTCCGTGTTGGCCAGGCTGGTCTCGAGACGACCCTGGCCAACAAGGAGAAACCC |
| 10 | GGGTTTCGCCATGTTGGCCAAGCTGGTCTCGAGACCAGCCTTGCCAACATGGTGAAACCC |
| 11 | GGGTTTCATCATGTTGGCCAGGCTGGTCTCGAGACCAGCCTGACCAACAGGTGAAACCCA |
| 12 | TCTCTTCCGGTTCTGTLTTTTCGCTGGCTCGAACAACAAATTGTTAAGTGAATGGAATGC |
| 13 | GGGTTTCACCATGTTGGCCAGACTGATCTCGAGACGATCCCGGCCAACATGGTGAAACCC |
| 14 | ACTGTAGGCAGACAGGGAACTTGGCATATCGAGGTGGTCCCACAAAAATATTTTATGTAG |
| 15 | TGGAGAAAACTGGTGGACACTACTTTAATCGAACTATTGGGCTCAAGCAATCCACCCACC |
| 16 | GGACATAACTGTATTCTCTCTCTCTCTCTCGAAGCTTTTTCCTGGGGCAGATTCTGCCTG |
| 17 | CATGATGCATAAACTCTAAACAGGTCTTTCGAGAAATGCCACGCTCACTAGAAAATATAG |
| 18 | GGGTTTCACCATGTTGGCCAGGCTGGTCTCGAGACCAGACTGGCCAACATAGTGAAACCC |
| 19 | CAGTCACAGCAAATGCCACAGGGTGTGTTCGAGGAAAAGTGGCAAATGTCTCTCTACTCC |
| 20 | TGATCTGGAAAAAAAATCAATTTAGATCTCGAAGCTGTCATCTGTGGGCGCGGGTTGAGC |
| 21 | GGATTTATCCGTGTTGGTCAGGCTGGTCTCGAGACAAGCCTGACTAACATGGTAAAACCC |
| 22 | GGACATAACTGTATTCTCTCTCTCTCTCTCGAGCAGGATGTGGAACAGACATCATGAGCC |
| 23 | TCTCTTCCGGTTCTGTCTTTTCGCTGGCTCGAGATAAGTGGACCCAGCTGTTTATTCCCA |
| 24 | CAGACTTTGGAACTAAGCTGACTTATATTCGAGATGGAGTTTTGCTCTTTTTGCCCAGGC |
| 25 | GGCAGGTGGACCACCTGAGGTCAGGAGTTCGAACTCCTGGACTCAAGTGATCTTCCCACC |
| 26 | GTAACAGCAGCAATGGCAGTGGTGGTGATCGAAATGCCCATGAAGTGAGCCAAAACTTTG |
| 27 | ACAACAACAAAAAAGAACCAAATAGTGATCGACTTGGGAGTGCCTGCGTTCCCGCGGCTC |
| 28 | GGGTTTCTCCATGTTGGTCAGGCTGGTCTCGAGACCAGTCTGACCAACATGGTGAAACCC |
| 29 | TTTGTAGTAAGTTTAATTAACTTAATTTTCGATACTTCCCCTGGGTGACTCTGACAGATG |
| 30 | GAAACACAACTACAAGGTGATGAGATTATCGATGGGGAGAGTCCAAGGATTTTAGCTGTG |
| 31 | TAGGGAAGTGGGTGGTGCTCAGGAGGTGTCGAGCCGAGGAGCCCCTCTGCCCTCCCTGTC |
| 32 | GGCAGGTGGATCATTTGAGGTCAGGAGTTCGAATTCCTGAGCTCAAATGATCCTCCTGCC |
| 33 | CACACCACCGTTGGGATGAAAGATGGCCTCGAAGTTGATGCAATCGGTTTAAACATGGCT |
| 34 | TGTGCCTTCACCCAAACTATCTGATCCCTCGACCCTTAAAAAAAAAAATTAGCCAGGCAT |
| 35 | CCTACCAGGAGTCCCCGCCACGCTCCCCTCGACGATGACACCATAACCCTGGCAACAAAA |
| 36 | TCCCTTGCTCTCTCTCTGTCTCTCAGATTCGAGCTCTTCTTCGGCGTCTGCAGCCTCTTC |
| 37 | AAGCCATGGTGGCAGCGCGGCGGTTGGTTCGAGTATCAGATAAATGAAAATGCTGAGACT |
| 38 | TGGACTCGGGGTGCCCGGTTCCACCCACTCGAAGTGATGGAACTCATAGGACCAGCTCAA |
| 39 | GGACATAACTGTATTCTCTCTCTCTCTCTCGAATTCCTGACCTCGGCCTTCCAAAGTGCT |
| 40 | GGATTTCATCATGTTGGCCAGGCTGGTCTCGAGACCAGTCTGGCCAACATGGTAAAACCC |
| 41 | TAGCAAAGCAGAGTTGGGGGTGCGATTGTCGAGGCCTAACAATTTTCCAGGGTCCAAAGG |
| 42 | AGGTTTCACCATGTTGGCCAGGCTGGTCTCGAGACCAACCTGGCCAACATGGTGCAACCC |
| 43 | CAACGCTCACGCCAATGGTCTGAGTTAATCGAATGCATCCTGGCCATGGTTCTGCAGCTC |
| 44 | TGGTGAGCAGAAGGCTCCAGCTGTACGCTCGATTCCTGTTTTCCCTTCTGCCTCCCTCGT |
| 45 | TTGCGCTCCAGGCCGGCTGCCATGGAGCTCGACCGCCAGGGCCCGCGTCCTCTCCCCGGC |
| 46 | AACAAGGACTTAAGTGTGTCCACTTATGTCGAGCAATCCTTCTGCCTCAGCCTCCTGAGT |
| 47 | TTCAAACAGTTTAGATACATCAAGTCCCTCGAGGCTGCAGAAGGCTGGGTGGGAGTGGAG |
| 48 | CTCTCAATAAAGTTTTCTAAAATTAGACTCGATGGTTCCCTATTACCCTCTGCCCGCAGC |
| 49 | GATGTCTTGGTTTTTCCATGAGAAACATTCGAGGTGCTCGGCGGGCAGCGCGGGGCTCAA |
| 50 | GGGTTTCACCATGTTGGTCAGGCTGGTCTCGAGACCAGCCTGAGCAACATGAAGAAACCC |
| 51 | TAGGGCTTTTACAGTTACAGAGGAGAAGTCGACGGAGTCGGTTCAGTTTGGGGCAGATCA |

TABLE 3.a4

| | Probe Location | | | | |
|---|---|---|---|---|---|
| | | Start1 | End1 | Start2 | End2 |
| 1 | 8 | 143363267 | 143363296 | 143397075 | 143397104 |
| 2 | X | 48754334 | 48754363 | 48801790 | 48801819 |
| 3 | 4 | 186175736 | 186175765 | 186192734 | 186192763 |
| 4 | 1 | 26205449 | 26205478 | 26333365 | 26333394 |
| 5 | 2 | 70888969 | 70888998 | 70981534 | 70981563 |
| 6 | 2 | 112539684 | 112539713 | 112816389 | 112816418 |
| 7 | 12 | 52040871 | 52040900 | 52237946 | 52237975 |
| 8 | 11 | 10562527 | 10562556 | 10617127 | 10617156 |
| 9 | 2 | 43092092 | 43092121 | 43179791 | 43179820 |
| 10 | 17 | 67419705 | 67419734 | 67604582 | 67604611 |
| 11 | 19 | 2777062 | 2777091 | 2905353 | 2905382 |
| 12 | 17 | 43300563 | 43300592 | 43444310 | 43444339 |
| 13 | 15 | 74687566 | 74687595 | 74772374 | 74772403 |
| 14 | 10 | 62056181 | 62056210 | 62096287 | 62096316 |
| 15 | 15 | 76885706 | 76885735 | 76985821 | 76985850 |
| 16 | 11 | 10641029 | 10641058 | 10678780 | 10678809 |
| 17 | 3 | 122771578 | 122771607 | 122793454 | 122793483 |
| 18 | 5 | 140251124 | 140251153 | 140387399 | 140387428 |
| 19 | 2 | 143128726 | 143128755 | 143274181 | 143274210 |
| 20 | 7 | 1769384 | 1769413 | 1921818 | 1921847 |
| 21 | 13 | 28589073 | 28589102 | 28625879 | 28625908 |
| 22 | 11 | 10641029 | 10641058 | 10826894 | 10826923 |
| 23 | 17 | 43300563 | 43300592 | 43353580 | 43353609 |
| 24 | 18 | 63921021 | 63921050 | 64068336 | 64068365 |
| 25 | 20 | 31989700 | 31989729 | 32045785 | 32045814 |
| 26 | 11 | 35047053 | 35047082 | 35078581 | 35078610 |
| 27 | 18 | 45766342 | 45766371 | 45837262 | 45837291 |
| 28 | 22 | 42716020 | 42716049 | 42864114 | 42864143 |
| 29 | 21 | 29043830 | 29043859 | 29094330 | 29094359 |
| 30 | X | 101622973 | 101623002 | 101717833 | 101717862 |
| 31 | 16 | 85536247 | 85536276 | 85655932 | 85655961 |
| 32 | 16 | 57546347 | 57546376 | 57811682 | 57811711 |
| 33 | 20 | 50192392 | 50192421 | 50281893 | 50281922 |
| 34 | 2 | 25160712 | 25160741 | 25292184 | 25292213 |
| 35 | 7 | 151781264 | 151781293 | 151930366 | 151930395 |
| 36 | 2 | 74503925 | 74503954 | 74521816 | 74521845 |
| 37 | 6 | 52576476 | 52576505 | 52715483 | 52715512 |
| 38 | 10 | 104325692 | 104325721 | 104442839 | 104442868 |
| 39 | 11 | 10376887 | 10376916 | 10641029 | 10641058 |
| 40 | 19 | 7273706 | 7273735 | 7367546 | 7367575 |
| 41 | 6 | 15165977 | 15166006 | 15249716 | 15249745 |
| 42 | 1 | 158187597 | 158187626 | 158252019 | 158252048 |
| 43 | 11 | 65046921 | 65046950 | 65063962 | 65063991 |
| 44 | 6 | 31267450 | 31267479 | 31534963 | 31534992 |
| 45 | 17 | 81104540 | 81104569 | 81345806 | 81345835 |
| 46 | 3 | 101887194 | 101887223 | 101950708 | 101950737 |
| 47 | 4 | 2639347 | 2639376 | 2800085 | 2800114 |
| 48 | 9 | 123262991 | 123263020 | 123339570 | 123339599 |
| 49 | 3 | 51244894 | 51244923 | 51391833 | 51391862 |
| 50 | 8 | 73772860 | 73772889 | 73996933 | 73996962 |
| 51 | 7 | 1901150 | 1901179 | 2038403 | 2038432 |

TABLE 3.a5

| | 4 kb Sequence Location | | | | |
|---|---|---|---|---|---|
| | | Start1 | End1 | Start2 | End2 |
| 1 | 8 | 143363267 | 143367266 | 143397075 | 143401074 |
| 2 | X | 48750364 | 48754363 | 48797820 | 48801819 |
| 3 | 4 | 186171766 | 186175765 | 186188764 | 186192763 |
| 4 | 1 | 26201479 | 26205478 | 26333365 | 26337364 |
| 5 | 2 | 70884999 | 70888998 | 70977564 | 70981563 |
| 6 | 2 | 112539684 | 112543683 | 112816389 | 112820388 |
| 7 | 12 | 52036901 | 52040900 | 52237946 | 52241945 |
| 8 | 11 | 10562527 | 10566526 | 10617127 | 10621126 |
| 9 | 2 | 43088122 | 43092121 | 43175821 | 43179820 |
| 10 | 17 | 67419705 | 67423704 | 67604582 | 67608581 |
| 11 | 19 | 2777062 | 2781061 | 2901383 | 2905382 |
| 12 | 17 | 43300563 | 43304562 | 43444310 | 43448309 |
| 13 | 15 | 74683596 | 74687595 | 74772374 | 74776373 |
| 14 | 10 | 62056181 | 62060180 | 62096287 | 62100286 |
| 15 | 15 | 76885706 | 76889705 | 76981851 | 76985850 |
| 16 | 11 | 10637059 | 10641058 | 10678780 | 10682779 |
| 17 | 3 | 122767608 | 122771607 | 122793454 | 122797453 |
| 18 | 5 | 140247154 | 140251153 | 140383429 | 140387428 |
| 19 | 2 | 143124756 | 143128755 | 143274181 | 143278180 |
| 20 | 7 | 1769384 | 1773383 | 1917848 | 1921847 |
| 21 | 13 | 28585103 | 28589102 | 28625879 | 28629878 |
| 22 | 11 | 10637059 | 10641058 | 10826894 | 10830893 |
| 23 | 17 | 43300563 | 43304562 | 43353580 | 43357579 |
| 24 | 18 | 63921021 | 63925020 | 64068336 | 64072335 |
| 25 | 20 | 31989700 | 31993699 | 32041815 | 32045814 |
| 26 | 11 | 35043083 | 35047082 | 35078581 | 35082580 |
| 27 | 18 | 45762372 | 45766371 | 45833292 | 45837291 |
| 28 | 22 | 42712050 | 42716049 | 42864114 | 42868113 |
| 29 | 21 | 29039860 | 29043859 | 29090360 | 29094359 |
| 30 | X | 101619003 | 101623002 | 101713863 | 101717862 |
| 31 | 16 | 85532277 | 85536276 | 85655932 | 85659931 |
| 32 | 16 | 57546347 | 57550346 | 57807712 | 57811711 |
| 33 | 20 | 50192392 | 50196391 | 50277923 | 50281922 |
| 34 | 2 | 25156742 | 25160741 | 25292184 | 25296183 |
| 35 | 7 | 151781264 | 151785263 | 151930366 | 151934365 |
| 36 | 2 | 74503925 | 74507924 | 74517846 | 74521845 |
| 37 | 6 | 52572506 | 52576505 | 52715483 | 52719482 |
| 38 | 10 | 104325692 | 104329691 | 104442839 | 104446838 |
| 39 | 11 | 10376887 | 10380886 | 10637059 | 10641058 |
| 40 | 19 | 7269736 | 7273735 | 7367546 | 7371545 |
| 41 | 6 | 15165977 | 15169976 | 15245746 | 15249745 |
| 42 | 1 | 158183627 | 158187626 | 158248049 | 158252048 |
| 43 | 11 | 65042951 | 65046950 | 65059992 | 65063991 |
| 44 | 6 | 31267450 | 31271449 | 31530993 | 31534992 |
| 45 | 17 | 81104540 | 81108539 | 81345806 | 81349805 |
| 46 | 3 | 101887194 | 101891193 | 101946738 | 101950737 |
| 47 | 4 | 2639347 | 2643346 | 2800085 | 2804084 |
| 48 | 9 | 123259021 | 123263020 | 123339570 | 123343569 |
| 49 | 3 | 51240924 | 51244923 | 51387863 | 51391862 |
| 50 | 8 | 73772860 | 73776859 | 73992963 | 73996962 |
| 51 | 7 | 1901150 | 1905149 | 2034433 | 2038432 |

TABLE 3.a6

| | Probe | Primer ID |
|---|---|---|
| 1 | ORF1_8_143363265_143368758_143397073_143399812_RR | OBD168-989 |
| 2 | ORF1_X_48750795_48754365_48796124_48801821_FF | OBD168-2109 |
| 3 | ORF1_4_186172696_186175767_186190828_186192765_FF | OBD168-669 |
| 4 | ORF1_1_26201425_26205480_26333363_26336159_FR | OBD168-1729 |
| 5 | ORF1_2_70887659_70889000_70979229_70981565_FF | OBD168-701 |
| 6 | ORF1_2_112539682_112540702_112816387_112823836_RR | OBD168-889 |
| 7 | ORF1_12_52037813_52040902_52237944_52243119_FR | OBD168-1141 |
| 8 | ORF1_11_10562525_10569392_10617125_10621846_RR | OBD168-1221 |
| 9 | ORF1_2_43086424_43092123_43178166_43179822_FF | OBD168-1905 |
| 10 | ORF1_17_67419703_67421985_67604580_67609081_RR | OBD168-1465 |
| 11 | ORF1_19_2777060_2780381_2901738_2905384_RF | OBD168-1873 |
| 12 | ORF1_17_43300561_43304860_43444308_43448547_RR | OBD168-1437 |
| 13 | ORF1_15_74686122_74687597_74772372_74779791_FR | OBD168-1841 |
| 14 | ORF1_10_62056179_62058893_62096285_62100217_RR | OBD168-905 |
| 15 | ORF1_15_76885704_76887654_76983002_76985852_RF | OBD168-705 |
| 16 | ORF1_11_10637008_10641060_10678778_10685244_FR | OBD168-865 |
| 17 | ORF1_3_122768838_122771609_122793452_122794847_FR | OBD168-1609 |
| 18 | ORF1_5_140242642_140251155_140386390_140387430_FF | OBD168-1641 |
| 19 | ORF1_2_143119651_143128757_143274179_143284059_FR | OBD168-1061 |
| 20 | ORF1_7_1769382_1772142_1917082_1921849_RF | OBD168-2053 |
| 21 | ORF1_13_28585301_28589104_28625877_28627714_FR | OBD168-1393 |
| 22 | ORF1_11_10637008_10641060_10826892_10830503_FR | OBD168-569 |
| 23 | ORF1_17_43300561_43304860_43353578_43358798_RR | OBD168-1433 |
| 24 | ORF1_18_63921019_63923125_64068334_64074049_RR | OBD168-1481 |
| 25 | ORF1_20_31989698_31994926_32038678_32045816_RF | OBD168-1953 |
| 26 | ORF1_11_35040602_35047084_35078579_35084103_FR | OBD168-1085 |
| 27 | ORF1_18_45762052_45766373_45829537_45837293_FF | OBD168-757 |
| 28 | ORF1_22_42713609_42716051_42864112_42866876_FR | OBD168-1965 |
| 29 | ORF1_21_29039171_29043861_29092829_29094361_FF | OBD168-697 |
| 30 | ORF1_X_101616906_101623004_101715574_101717864_FF | OBD168-977 |
| 31 | ORF1_16_85533210_85536278_85655930_85660237_FR | OBD168-1117 |
| 32 | ORF1_16_57546345_57549019_57808053_57811713_RF | OBD168-1861 |
| 33 | ORF1_20_50192390_50194162_50280026_50281924_RF | OBD168-849 |
| 34 | ORF1_2_25155910_25160743_25292182_25293890_FR | OBD168-557 |
| 35 | ORF1_7_151781262_151783710_151930364_151932111_RR | OBD168-765 |
| 36 | ORF1_2_74503923_74507137_74519716_74521847_RF | OBD168-1541 |
| 37 | ORF1_6_52573576_52576507_52715481_52719514_FR | OBD168-773 |
| 38 | ORF1_10_104325690_104332059_104442837_104447074_RR | OBD168-1353 |
| 39 | ORF1_11_10376885_10382511_10637008_10641060_RF | OBD168-573 |
| 40 | ORF1_19_7270757_7273737_7367544_7370291_FR | OBD168-1489 |
| 41 | ORF1_6_15165975_15168155_15248586_15249747_RF | OBD168-737 |
| 42 | ORF1_1_158181667_158187628_158243613_158252050_FF | OBD168-1325 |
| 43 | ORF1_11_65044650_65046952_65060361_65063993_FF | OBD168-837 |
| 44 | ORF1_6_31267448_31269252_31531115_31534994_RF | OBD168-805 |
| 45 | ORF1_17_81104538_81109597_81345804_81351809_RR | OBD168-1869 |
| 46 | ORF1_3_101887192_101889071_101943010_101950739_RF | OBD168-1981 |
| 47 | ORF1_4_2639345_2642002_2800083_2804902_RR | OBD168-1997 |
| 48 | ORF1_9_123259504_123263022_123339568_123345302_FR | OBD168-529 |
| 49 | ORF1_3_51243406_51244925_51388915_51391864_FF | OBD168-1605 |
| 50 | ORF1_8_73772858_73775783_73991731_73996964_RF | OBD168-2089 |
| 51 | ORF1_7_1901148_1902503_2033188_2038434_RF | OBD168-1677 |

TABLE 3.a7

| | Sequence | Primer ID | Sequence |
|---|---|---|---|
| 1 | CAGGCTGTCTCATTCTGTTGCCC | OBD168-991 | GCCACATACAGTCAGGGTCCAGC |
| 2 | TTAGGAGTGTCCCAAACCCCC | OBD168-2111 | TTTACGTAGGCGCAGCACAGC |
| 3 | AAGAAACAAAGCGAGGCAAGGTGACG | OBD168-671 | AGGCAGACTTCTTTGGAGGGTGTCAA |
| 4 | ATCTTGGCTCACTGCAACCTCC | OBD168-1731 | GCTGGAGTGCAATGGCGCAATC |
| 5 | TCGCTCATTCCTCCCGACCAAGG | OBD168-703 | CTGAAGACAGAGTGAGCCAGCCA |
| 6 | TCATCATACTTCTCCCTTTCCAATCT | OBD168-891 | TCTGCCAGCCCTAATCCCTCTGAATA |
| 7 | CCTCCTCCTCTTTGCCCCTCCTA | OBD168-1143 | GGTGAAGATGGTGTCTGAGCCTC |
| 8 | GACACCTGGAGGGCAGCACTGAT | OBD168-1223 | GGTAGTTGGCTATCTGGACCTGG |
| 9 | TGGCACAGTCTCAGCTCACTG | OBD168-1907 | TCTCGTTCTGTTGCCCAGGCT |
| 10 | CTGGGTCTCCAGCCTCACTCAGG | OBD168-1467 | CTGGAGTGCAGTGGCAAGTTCTC |
| 11 | GTCTTGCTTTGTTGCTCAGGTTGGAG | OBD168-1875 | GCGCCTGGTCTTTTTTTTTTTTTCTG |
| 12 | CGGGTACGGGGCCGGTCTCCCCGCCC | OBD168-1439 | AAACGAAGACATGAGTCACTAGTAAG |
| 13 | CTCACTGCAACTTCTGCCTCCCAG | OBD168-1843 | ATCCTTTTTTTTTTTTTCTGAGAC |
| 14 | TTGCCACCCAAGAGTGATGCGGG | OBD168-907 | GGCTGACCACCACCAAATGTGAC |
| 15 | CTCAAAGGATTTCCCCACAAGATACT | OBD168-707 | GCTTTTCTTTGTTTGGTAGGGTTATG |
| 16 | GGATAAAGCACAGGATGCCCCAGTTA | OBD168-867 | GAAGGTGACAGAAGACCCAGAAAAGG |
| 17 | CTGAGCTTGCTTTTAATTATTATAAA | OBD168-1611 | CCTGAGTACAAACCCAATGCCAGTTG |
| 18 | ATGATCTAGGCTCACTGCAAACT | OBD168-1643 | TGCCTCCTGGGTTCAAGCGATTC |
| 19 | TGGACACATCCGTATGAGAGCCACAG | OBD168-1063 | AACAGCCAGGCACCATTGAACATTTC |
| 20 | TAAATGAAATTAAAAAAAACAAA | OBD168-2055 | CTCGGCTCCGCAAAGACATTCTG |
| 21 | GAGTTTCACTCTTGTTGCCCA | OBD168-1395 | CGCTCTGTCATGCAGGCTGAA |
| 22 | GGATAAAGCACAGGATGCCCCAGTTA | OBD168-571 | TACACTGTCTCACATTTCTCCCTGGC |
| 23 | CGTCCCCTCGTCCCCTCGGGT | OBD168-1435 | AGAGACTCTTGTTACAACCCA |
| 24 | TCATTAATATTCATTAATGAC | OBD168-1483 | ACTGGAGCATGCCTGTAATCC |
| 25 | AGCCAGACAGATCCTGTGAAAAGAT | OBD168-1955 | ATGCCTGGAATCCCAACATTTTGGG |
| 26 | GACTGGAGTGCTCTGCTCATCTC | OBD168-1087 | CACCCAAAGTCCCCACCTGCTCA |
| 27 | CCTACCATTGCCCACTTGAGAGGG | OBD168-759 | TCCACAGGGCAGGTTTTGATGGG |
| 28 | CCTGCGTTCAAGCGATTCTTCTG | OBD168-1967 | TGGGTTCAAGCGATTCTCCTGCC |
| 29 | GCTATTGAGTTTGTAGTCTGAATGAC | OBD168-699 | GCAGTGGGTTCTCAAATGTTAGCATC |
| 30 | CGTCCCTTTCTGGTAATCGGTGGAAG | OBD168-979 | CTTCATTTCCATTTCCTGACACACAG |
| 31 | GGTCACCAACAAGGCTTCTGTGG | OBD168-1119 | AGTCCAGTCAGGTTCCAGCCCAG |
| 32 | GGGTATGGAGTGCATACCTATGGTC | OBD168-1863 | AGACAAGAGCTGATTTTCCCATTAT |
| 33 | CTTGAAGGCGGATTCCTCCAGGT | OBD168-851 | GTGGGTTGCGTCAGTCCCGTGTA |
| 34 | TCAGTGGAGTGTGTGGAAAATACGGC | OBD168-559 | CCCTTCTTACCAAACCTGTCACCCTA |
| 35 | GCTCTCCCAAAACCGTGTTCCCG | OBD168-767 | CCCTGTTTCTGTCCTCCTCCACT |
| 36 | GCTCTCTCTCTCTCCCTCTCTTTCCC | OBD168-1543 | AGAGTGAGTACAGCGAAGGCGGCCTC |
| 37 | TGGACGCCAGAAAGGGAACCCCA | OBD168-775 | AGAATCACTCAGCCGAGCCCTTC |
| 38 | GCATCCCACTCAGTCAGTCTGGGC | OBD168-1355 | AACTATGCCCATCGTATTTTACCA |
| 39 | GGATAAAGCACAGGATGCCCCAGTTA | OBD168-575 | GTCACACAACAGAGACAGACTTGATA |
| 40 | GGTTCAAGCAATTCTCCTGCCTCAG | OBD168-1491 | TCCTGGGTTCAAGTGAGTCTCCTGC |
| 41 | CGGGATGCCTGCTGAGTTGCCAT | OBD168-739 | CCGCTGAGGTCTAAGGGTTTCCA |
| 42 | GGCGTGGTCTCGGCTCACTGCA | OBD168-1327 | CGGCTCACTGCAACCTCCGCCT |
| 43 | GCATCAAACAGGGAGGTGCCAGC | OBD168-839 | CCTCAGGACAGCCAGTGAAGGAT |
| 44 | TGTCCCTCTCTTGCTCTCCTGCC | OBD168-807 | CTCTGATGGCTGTGCTGAGGACA |
| 45 | CCCCCGAGAAACACTCACATCTTGG | OBD168-1871 | CCCCGGGGCTCCTGCCCGGACGGCG |
| 46 | TCTCAGTAACTTACATTTTCAGTTTG | OBD168-1983 | AATCCCAACACTTTGGGAGGCCGAGG |
| 47 | CTAAAATGGCAAGTTTAGAAGGGC | OBD168-1999 | GTCTGCCAGAAAAGCCAGCCAAGG |
| 48 | CACTTGGTCACCTCACTATTCAATGC | OBD168-531 | CGGTTCGCTTTCCCCTGGTTATGC |
| 49 | TTCTGATCTTAGAGGAAATGCTTTC | OBD168-1607 | GACGCGGCGGCAAGGCCTCGGGGGA |
| 50 | CCTCAGCCTTCTGAGTAGCAGGGATT | OBD168-2091 | TTCTTTTGACAGAATCTCACTCTTAC |
| 51 | ACCAATGAAGGTGGCCACACTAAAC | OBD168-1679 | CCAGAGGAACCCCGGGCTCTTGGCT |

TABLE 3.a8

| | qPCR Primer ID | Sequence | qPCR Primer ID |
|---|---|---|---|
| 1 | OBD168-q989 | GGAGGAGGCTGGTGGAGACCCTGTTGGA | OBD168-q991 |
| 2 | OBD168-q2109 | GTGTGATTTCAGGGGTGCCTGCC | OBD168-q2111 |
| 3 | OBD168-q669 | TGAAGAAACAAAGCGAGGCAAGGTGACG | OBD168-q671 |
| 4 | OBD168-q1729 | GATCTTGGCTCACTGCAACCT | OBD168-q1731 |
| 5 | OBD168-q701 | TCGCCCCTCGCTCATTCCTCCCGACCAA | OBD168-q703 |
| 6 | OBD168-q889 | TCATCATACTTCTCCCTTTCCAATCTGA | OBD168-q891 |
| 7 | OBD168-q1141 | GGCAAAGGTCAGGTGGGTGAGGAAGCCC | OBD168-q1143 |
| 8 | OBD168-q1221 | GGAGGGCAGCACTGATTTGTTCGTGGTC | OBD168-q1223 |
| 9 | OBD168-q1905 | GGCCTCCCGAGTAGCTGGGATTACAA | OBD168-q1907 |
| 10 | OBD168-q1465 | TGCCTCAGCCTCCCGAGTAGCT | OBD168-q1467 |
| 11 | OBD168-q1873 | TTCAAGCGATTCTTCTGCCTCAGCCT | OBD168-q1875 |
| 12 | OBD168-q1437 | GCGGGCGGCGAAGTAAAGGCCCA | OBD168-q1439 |
| 13 | OBD168-q1841 | TCTCCTTCCTCAGCCTCCTGAG | OBD168-q1843 |
| 14 | OBD168-q905 | GCTGTTTGCCACCCAAGAGTGATGCGGG | OBD168-q907 |
| 15 | OBD168-q705 | ATAACAGAAGGGAACAGAAGCCACCC | OBD168-q707 |
| 16 | OBD168-q865 | CACACGCATCCCTGCTCCCATTCTGAG | OBD168-q867 |
| 17 | OBD168-q1609 | TATAAAAAGGGGTCCTAAAATAGTT | OBD168-q1611 |
| 18 | OBD168-q1641 | CAATTCTCCCATCTCAGCCTCCCGAG | OBD168-q1643 |
| 19 | OBD168-q1061 | CCAGAGTGGACACATCCGTATGAGAGCC | OBD168-q1063 |
| 20 | OBD168-q2053 | AATACAGACCCGATTTTGCCT | OBD168-q2055 |
| 21 | OBD168-q1393 | TCTTCTGCCTCGGCCTCTGGA | OBD168-q1395 |
| 22 | OBD168-q569 | CACACGCATCCCTGCTCCCATTCTGAG | OBD168-q571 |
| 23 | OBD168-q1433 | GTACGGGGCCGGTCTCCCCGCCCG | OBD168-q1435 |
| 24 | OBD168-q1481 | AATTAATCTTCATTAAGTCATG | OBD168-q1483 |
| 25 | OBD168-q1953 | AGATCCTGTGAAAAGATAAGCCAGTT | OBD168-q1955 |
| 26 | OBD168-q1085 | CCCACCTCCTAACCATCACCTCCTCTCC | OBD168-q1087 |
| 27 | OBD168-q757 | CTTTGCGTGTCTGTGGCTCTGTGGCTCC | OBD168-q759 |
| 28 | OBD168-q1965 | TTTCGCTCTTTTTGCCCAGGCTGGA | OBD168-q1967 |
| 29 | OBD168-q697 | GCTATTGAGTTTGTAGTCTGAATGACAT | OBD168-q699 |
| 30 | OBD168-q977 | TGCCGTCCCTTTCTGGTAATCGGTGGAA | OBD168-q979 |
| 31 | OBD168-q1117 | CTGACGGGCATTCATTGGGCACCTGCTG | OBD168-q1119 |
| 32 | OBD168-q1861 | CTGGGTATGGAGTGCATACCTAT | OBD168-q1863 |
| 33 | OBD168-q849 | GCTTGCTTGAAGGCGGATTCCTCCAGG | OBD168-q851 |
| 34 | OBD168-q557 | GACAGTCCAGAGCACTCCCAGGCGTGG | OBD168-q559 |
| 35 | OBD168-q765 | TACAGGGCAGGAGGCAGCGTGTCCAGAG | OBD168-q767 |
| 36 | OBD168-q1541 | TCCCTCTCTTTCCCTCTCCCTCTCTC | OBD168-q1543 |
| 37 | OBD168-q773 | ACTGCGGGTCTGGGAGTCTCTGGGCTT | OBD168-q775 |
| 38 | OBD168-q1353 | ACAGGCCTTACTCTAATACCAAGCGC | OBD168-q1355 |
| 39 | OBD168-q573 | GATTTCTGGATAAAGCACAGGATGCCCC | OBD168-q575 |
| 40 | OBD168-q1489 | TCTGGGGTTCAAGCAATTCTCCTG | OBD168-q1491 |
| 41 | OBD168-q737 | CGGGATGCCTGCTGAGTTGCCATTTAGT | OBD168-q739 |
| 42 | OBD168-q1325 | CAACCTCCACCTCCTGGGTTCAAGT | OBD168-q1327 |
| 43 | OBD168-q837 | GGCTTGGGATGGGAAGTGGGAGAAGGTG | OBD168-q839 |
| 44 | OBD168-q805 | CGCTGCCGCCATCCACCGCTGGGT | OBD168-q807 |
| 45 | OBD168-q1869 | TAATGAGGTCACCCTCCTTGAA | OBD168-q1871 |
| 46 | OBD168-q1981 | GACCTTCTCAGTAACTTACATTT | OBD168-q1983 |
| 47 | OBD168-q1997 | AGACAGGGTGACAGGTGTGCA | OBD168-q1999 |
| 48 | OBD168-q529 | GTGGAGAGTCTGTTGTGAGGCAGGCAC | OBD168-q531 |
| 49 | OBD168-q1605 | ATTTCTGATCTTAGAGGAAATGCT | OBD168-q1607 |
| 50 | OBD168-q2089 | ATCTTGGCTCACAGCAGTCTCTG | OBD168-q2091 |
| 51 | OBD168-q1677 | GTCCCCATCACATAAAAGTAC | OBD168-q1679 |

TABLE 3.a9

| | Sequence | qPCR Probe |
|---|---|---|
| 1 | AAGAGCAGGGCTGGAGGCAGGCG | OBD168-p989 |
| 2 | GAGCTCGCGGTGTGGCTCAGCCC | OBD168-p2109 |
| 3 | GCCGCCCTCCTCTCGCTGCCACT | OBD168-p669 |
| 4 | CTCTTGTTGCCTAGGCTGGAG | OBD168-p1729 |
| 5 | GGAACCTGAAGACAGAGTGAGCCAGCC | OBD168-p701 |
| 6 | TTCTGCCAGCCCTAATCCCTCTGAATAC | OBD168-p889 |
| 7 | CCAGCCCCGCTCCCAGAGATGGA | OBD168-p1141 |
| 8 | GGCTATCTGGACCTGGATTTTGAGGGAC | OBD168-p1221 |
| 9 | GCAGTGGGGCAATCTTGGCTTACTGC | OBD168-p1905 |
| 10 | CGCCTCCTGGGTTCAAGTGATT | OBD168-p1465 |
| 11 | CTCTTATTGCCCAGGCTGGAGTACAG | OBD168-p1873 |
| 12 | GAAGACATGAGTCACTAGTAAGG | OBD168-p1437 |
| 13 | ATATCAGCTCACTGCAACCTCT | OBD168-p1841 |
| 14 | CCAGATGCGACTTGGCTGACCACCACC | OBD168-p905 |
| 15 | GCTTTTCTTTGTTTGGTAGGGTTATGAT | OBD168-p705 |
| 16 | CAGAAGGTGACAGAAGACCCAGAAAAGG | OBD168-p865 |
| 17 | GGAGGAAGGTAGGTGCCAGGCTGAG | OBD168-p1609 |
| 18 | AGCTCACTGTAACCTCTGCCTCCTGG | OBD168-p1641 |
| 19 | GCTTGTGTGGAACAGCCAGGCACCATTG | OBD168-p1061 |
| 20 | CACAGACACCGACCGGAACGG | OBD168-p2053 |
| 21 | GTGTGGTGGCGCCATCTCAGC | OBD168-p1393 |
| 22 | GTTACACTGTCTCACATTTCTCCCTGGC | OBD168-p569 |
| 23 | TGGAAACCAAAGACTCCCTTCCCC | OBD168-p1433 |
| 24 | GAGTTCAAGACCAGCCTGACCA | OBD168-p1481 |
| 25 | TGTGGCGGCTCATGCCTGGAATCCCA | OBD168-p1953 |
| 26 | GTCACCCAAAGTCCCCACCTGCTCATAC | OBD168-p1085 |
| 27 | ACGGAGAACTTGCTCAACACAGAGGTGC | OBD168-p757 |
| 28 | CCTGGGTTCAAGCGATTCTCCTGCC | OBD168-p1965 |
| 29 | CAGCAGTGGGTTCTCAAATGTTAGCATC | OBD168-p697 |
| 30 | GTCTTCATTTCCATTTCCTGACACACAG | OBD168-p977 |
| 31 | AGGAGTCCAGTCAGGTTCCAGCCCAGG | OBD168-p1117 |
| 32 | TTATGTAAAAGTTTCTAGAAATT | OBD168-p1861 |
| 33 | CGTGGGTTGCGTCAGTCCCGTGTA | OBD168-p849 |
| 34 | GGGCAGAGGGAGGACACCCATTAGAAAT | OBD168-p557 |
| 35 | GGCTTCTCCCTGTTTCTGTCCTCCTCC | OBD168-p765 |
| 36 | AACTCCAGACTTGCGGGAAAGGCCTG | OBD168-p1541 |
| 37 | GCAAGAATCACTCAGCCGAGCCCTTCCC | OBD168-p773 |
| 38 | GCAAACTATGCCCATCGTATTTTACC | OBD168-p1353 |
| 39 | GTCACACAACAGAGACAGACTTGATAAT | OBD168-p573 |
| 40 | GGGTTCAAGTGAGTCTCCTGCCTC | OBD168-p1489 |
| 41 | CCTTCCCTTCTGGCTCCCTCTTTACCCC | OBD168-p737 |
| 42 | CCTTTCCCCACCAGGTTAGGGCTTT | OBD168-p1325 |
| 43 | TCTGGGCTGGGCTGGGACTGCTTGGAT | OBD168-p837 |
| 44 | CAGGAGTGGTCAATGTGTGCCTTGTTGC | OBD168-p805 |
| 45 | CTGCCCGGACGGCGGCCGCCGC | OBD168-p1869 |
| 46 | AGGCCAGGAGTTCAAGACCCTAG | OBD168-p1981 |
| 47 | GGCCAATGCCACCCCAATCTC | OBD168-p1997 |
| 48 | GGAGAGAGCCAGTGGTGAGAAACGCAGC | OBD168-p529 |
| 49 | GCCCCGCGCGTCTCCTCTGCCGCC | OBD168-p1605 |
| 50 | TTTTTTTGTTTTCTTTTGACAGA | OBD168-p2089 |
| 51 | GGGCTCTTGGCTCACTTGGGA | OBD168-p1677 |

TABCE 3.a10

| | Sequence | qPCR Probe |
|---|---|---|
| 1 | TAGGCTGGTCTCGAGGGAACTCCCTCCCC | OBD168-p991 |
| 2 | TGAGTGACTCTAGCGATGTCGACTGCCCGCCCAGATCCCG | OBD168-p2111 |
| 3 | AGGAGCTGCAACTTTCCATCGAGTTCGCCCTC | OBD168-p671 |
| 4 | ATGTTGGTCAGGCTGGTCTCGAGGCCAGCCTGACCAACAT | OBD168-p1731 |
| 5 | AGAGGGCGCGGCCTACACTCGATCTTATCCTT | OBD168-p703 |
| 6 | TCCTTTTGTCGAACTCCTAACCTTGTGATCTACCCGC | OBD168-p891 |
| 7 | TGCGTAGTCGAGCCAGCATGAGCTCTCTGTTC | OBD168-p1143 |
| 8 | TCATTCTCGATGTACTTCTCTGCCTCAGCCTACGTG | OBD168-p1223 |
| 9 | GTGTTGGCCAGGCTGGTCTCGAGACGACCCTGGCCAACAA | OBD168-p1907 |
| 10 | ATGTTGGCCAAGCTGGTCTCGAGACCAGCCTTGCCAACAT | OBD168-p1467 |
| 11 | ATGTTGGCCAGGCTGGTCTCGAGACCAGCCTGACCAACAG | OBD168-p1875 |
| 12 | TTCTGTCTTTTCGCTGGCTCGAACAACAAATTGTTAAGTG | OBD168-p1439 |
| 13 | ATGTTGGCCAGACTGATCTCGAGACGATCCCGGCCAACAT | OBD168-p1843 |
| 14 | AGGCAGACAGGGAACTTGGCATATCGAGGTGG | OBD168-p907 |
| 15 | TGGTGGACACTACTTTAATCGAACTATTGGGCTCAAGC | OBD168-p707 |
| 16 | TGGACATAACTGTATTCTCTCTCTCTCTCTCGAAGCT | OBD168-p867 |
| 17 | AAACTCTAAACAGGTCTTTCGAGAAATGCCACGCTCACTA | OBD168-p1611 |
| 18 | ATGTTGGCCAGGCTGGTCTCGAGACCAGACTGGCCAACAT | OBD168-p1643 |
| 19 | AGTCACAGCAAATGCCACAGGGTGTGTTCGAGGAA | OBD168-p1063 |
| 20 | AAAAAATCAATTTAGATCTCGAAGCTGTCATCTGTGGGCG | OBD168-p2055 |
| 21 | GTGTTGGTCAGGCTGGTCTCGAGACAAGCCTGACTAACAT | OBD168-p1395 |
| 22 | TCTCTCTCTCTCTCTCGAGCAGGATGTGGAACAG | OBD168-p571 |
| 23 | TTCTGTCTTTTCGCTGGCTCGAGATAAGTGGACCCAGCTG | OBD168-p1435 |
| 24 | AACTAAGCTGACTTATATTCGAGATGGAGTTTTGCTCTTT | OBD168-p1483 |
| 25 | CCACCTGAGGTCAGGAGTTCGAACTCCTGGACTCAAGTGA | OBD168-p1955 |
| 26 | AGCAGCAATGGCAGTGGTGGTGATCGAAATGCC | OBD168-p1087 |
| 27 | ATAGTGATCGACTTGGGAGTGCCTGCGTTCCC | OBD168-p759 |
| 28 | ATGTTGGTCAGGCTGGTCTCGAGACCAGTCTGACCAACAT | OBD168-p1967 |
| 29 | ACTTAATTTTCGATACTTCCCCTGGGTGACTCTGACAG | OBD168-p699 |
| 30 | ACACAACTACAAGGTGATGAGATTATCGATGGG | OBD168-p979 |
| 31 | TGGTGCTCAGGAGGTGTCGAGCCGAGGA | OBD168-p1119 |
| 32 | TCATTTGAGGTCAGGAGTTCGAATTCCTGAGCTCAAATGA | OBD168-p1863 |
| 33 | ACCACCGTTGGGATGAAAGATGGCCTCGAAGTTG | OBD168-p851 |
| 34 | TGCCTTCACCCAAACTATCTGATCCCTCGACCCT | OBD168-p559 |
| 35 | ACGCTCCCCTCGACGATGACACCATAACCCTG | OBD168-p767 |
| 36 | TCTCTCTGTCTCTCAGATTCGAGCTCTTCTTCGGCGTCTG | OBD168-p1543 |
| 37 | TGGCAGCGCGGCGGTTGGTTCGAGTATCA | OBD168-p775 |
| 38 | GTGCCCGGTTCCACCCACTCGAAGTGATGGAACTCATAGG | OBD168-p1355 |
| 39 | TCTCTCTCTCTCTCGAATTCCTGACCTCGGCC | OBD168-p575 |
| 40 | ATGTTGGCCAGGCTGGTCTCGAGACCAGTCTGGCCAACAT | OBD168-p1491 |
| 41 | TGCGATTGTCGAGGCCTAACAATTTTCCAGGGTCC | OBD168-p739 |
| 42 | ATGTTGGCCAGGCTGGTCTCGAGACCAACCTGGCCAACAT | OBD168-p1327 |
| 43 | ACGCCAATGGTCTGAGTTAATCGAATGCATCCTGGC | OBD168-p839 |
| 44 | AGCAGAAGGCTCCAGCTGTACGCTCGATTCCT | OBD168-p807 |
| 45 | GGCCGGCTGCCATGGAGCTCGACCGCCAGGGCCCGCGTCC | OBD168-p1871 |
| 46 | TAAGTGTGTCCACTTATGTCGAGCAATCCTTCTGCCTCAG | OBD168-p1983 |
| 47 | TTAGATACATCAAGTCCCTCGAGGCTGCAGAAGGCTGGGT | OBD168-p1999 |
| 48 | TTAGACTCGATGGTTCCCTATTACCCTCTGCCCG | OBD168-p531 |
| 49 | TTTTTCCATGAGAAACATTCGAGGTGCTCGGCGGGCAGCG | OBD168-p1607 |
| 50 | ATGTTGGTCAGGCTGGTCTCGAGACCAGCCTGAGCAACAT | OBD168-p2091 |
| 51 | ACAGTTACAGAGGAGAAGTCGACGGAGTCGGTTCAGTTTG | OBD168-p1679 |

TABLE 3.a11

| | Sequence | probe |
|---|---|---|
| 1 | AGGGAGTTCCCTCGAGACCAGCCTAGG | ORF1_8_143363265_143368758_143397073_143399812_RR |
| 2 | CGGGATCTGGGCGGGCAGTCGACATCGCTAGAGTCACTCA | ORF1_X_48750795_48754365_48796124_48801821_FF |
| 3 | AGGGAGGGCGAACTCGATGGAAAGTTGCAGCT | ORF1_4_186172696_186175767_186190828_186192765_FF |
| 4 | ATGTTGGTCAGGCTGGCCTCGAGACCAGCCTGACCAACAT | ORF1_1_26201425_26205480_26333363_26336159_FR |
| 5 | AAGGATAAGATCGAGTGTAGGCCGCGCCCTCT | ORF1_2_70887659_70889000_70979229_70981565_FF |
| 6 | AGGCGGGTAGATCACAAGGTTAGGAGTTCGACAAAAG | ORF1_2_112539682_112540702_112816387_112823836_RR |
| 7 | AACAGAGAGCTCATGCTGGCTCGACTACGCAC | ORF1_12_52037813_52040902_52237944_52243119_FR |
| 8 | ACGTAGGCTGAGGCAGAGAAGTACATCGAGAATGATC | ORF1_11_10562525_10569392_10617125_10621846_RR |
| 9 | TTGTTGGCCAGGGTCGTCTCGAGACCAGCCTGGCCAACAC | ORF1_2_43086424_43092123_43178166_43179822_FF |
| 10 | ATGTTGGCAAGGCTGGTCTCGAGACCAGCTTGGCCAACAT | ORF1_17_67419703_67421985_67604580_67609081_RR |
| 11 | CTGTTGGTCAGGCTGGTCTCGAGACCAGCCTGGCCAACAT | ORF1_19_2777060_2780381_2901738_2905384_RF |
| 12 | CACTTAACAATTTGTTGTTCGAGCCAGCGAAAAGACAGAA | ORF1_17_43300561_43304860_43444308_43448547_RR |
| 13 | ATGTTGGCCGGGATCGTCTCGAGATCAGTCTGGCCAACAT | ORF1_15_74686122_74687597_74772372_74779791_FR |
| 14 | ACCACCTCGATATGCCAAGTTCCCTGTCTGCC | ORF1_10_62056179_62058893_62096285_62100217_RR |
| 15 | TGCTTGAGCCCAATAGTTCGATTAAAGTAGTGTCCACC | ORF1_15_76885704_76887654_76983002_76985852_RF |
| 16 | AGCTTCGAGAGAGAGAGAGAGAATACAGTTATGTCC | ORF1_11_10637008_10641060_10678778_10685244_FR |
| 17 | TAGTGAGCGTGGCATTTCTCGAAAGACCTGTTTAGAGTTT | ORF1_3_122768838_122771609_122793452_122794847_FR |
| 18 | ATGTTGGCCAGTCTGGTCTCGAGACCAGCCTGGCCAACAT | ORF1_5_140242642_140251155_140386390_140387430_FF |
| 19 | TTCCTCGAACACACCCTGTGGCATTTGCTGTGAC | ORF1_2_143119651_143128757_143274179_143284059_FR |
| 20 | CGCCCACAGATGACAGCTTCGAGATCTAAATTGATTTTTT | ORF1_7_1769382_1772142_1917082_1921849_RF |
| 21 | ATGTTAGTCAGGCTTGTCTCGAGACCAGCCTGACCAACAC | ORF1_13_28585301_28589104_28625877_28627714_FR |
| 22 | TCTGTTCCACATCCTGCTCGAGAGAGAGAGAGAG | ORF1_11_10637008_10641060_10826892_10830503_FR |
| 23 | CAGCTGGGTCCACTTATCTCGAGCCAGCGAAAAGACAGAA | ORF1_17_43300561_43304860_43353578_43358798_RR |
| 24 | AAAGAGCAAAACTCCATCTCGAATATAAGTCAGCTTAGTT | ORF1_18_63921019_63923125_64068334_64074049_RR |
| 25 | TCACTTGAGTCCAGGAGTTCGAACTCCTGACCTCAGGTGG | ORF1_20_31989698_31994926_32038678_32045816_RF |
| 26 | TGGGCATTTCGATCACCACCACTGCCATTGCTG | ORF1_11_35040602_35047084_35078579_35084103_FR |
| 27 | ACGCAGGCACTCCCAAGTCGATCACTATTTGGTTC | ORF1_18_45762052_45766373_45829537_45837293_FF |
| 28 | ATGTTGGTCAGACTGGTCTCGAGACCAGCCTGACCAACAT | ORF1_22_42713609_42716051_42864112_42866876_FR |
| 29 | CTGTCAGAGTCACCCAGGGGAAGTATCGAAAATTAAGT | ORF1_21_29039171_29043861_29092829_29094361_FF |
| 30 | TCCTTGGACTCTCCCCATCGATAATCTCATCACCTTG | ORF1_X_101616906_101623004_101715574_101717864_FF |
| 31 | TCCTCGGCTCGACACCTCCTGAGCACCA | ORF1_16_85533210_85536278_85655930_85660237_FR |
| 32 | TCATTTGAGCTCAGGAATTCGAACTCCTGACCTCAAATGA | ORF1_16_57546345_57549019_57808053_57811713_RF |
| 33 | AACTTCGAGGCCATCTTTCATCCCAACGGTGGTG | ORF1_20_50192390_50194162_50280026_50281924_RF |
| 34 | AGGGTCGAGGGATCAGATAGTTTGGGTGAAGGC | ORF1_2_25155910_25160743_25292182_25293890_FR |
| 35 | CAGGGTTATGGTGTCATCGTCGAGGGGAGCGT | ORF1_7_151781262_151783710_151930364_151932111_RR |
| 36 | CAGACGCCGAAGAAGAGCTCGAATCTGAGAGACAGAGAGA | ORF1_2_74503923_74507137_74519716_74521847_RF |
| 37 | TTATCTGATACTCGAACCAACCGCCGCGCTGC | ORF1_6_52573576_52576507_52715481_52719514_FR |
| 38 | CCTATGAGTTCCATCACTTCGAGTGGGTGGAACCGGGCAC | ORF1_10_104325690_104332059_104442837_104447074_RR |
| 39 | AGGCCGAGGTCAGGAATTCGAGAGAGAGAGAG | ORF1_11_10376885_10382511_10637008_10641060_RF |
| 40 | ATGTTGGCCAGACTGGTCTCGAGACCAGCCTGGCCAACAT | ORF1_19_7270757_7273737_7367544_7370291_FR |
| 41 | ACCCTGGAAAATTGTTAGGCCTCGACAATCGCACC | ORF1_6_15165975_15168155_15248586_15249747_RF |
| 42 | ATGTTGGCCAGGTTGGTCTCGAGACCAGCCTGGCCAACAT | ORF1_1_158181667_158187628_158243613_158252050_FF |
| 43 | TGGCCAGGATGCATTCGATTAACTCAGACCATTGGC | ORF1_11_65044650_65046952_65060361_65063993_FF |
| 44 | AGGAATCGAGCGTACAGCTGGAGCCTTCTGCT | ORF1_6_31267448_31269252_31531115_31534994_RF |
| 45 | GGACGCGGGCCCTGGCGGTCGAGCTCCATGGCAGCCGGCC | ORF1_17_81104538_81109597_81345804_81351809_RR |
| 46 | CTGAGGCAGAAGGATTGCTCGACATAAGTGGACACACTTA | ORF1_3_101887192_101889071_101943010_101950739_RF |
| 47 | ACCCAGCCTTCTGCAGCCTCGAGGGACTTGATGTATCTAA | ORF1_4_2639345_2642002_2800083_2804902_RR |
| 48 | TGCGGGCAGAGGGTAATAGGGAACCATCGAGTCTAA | ORF1_9_123259504_123263022_123339568_123345302_FR |
| 49 | CGCTGCCCGCCGAGCACCTCGAATGTTTCTCATGGAAAAA | ORF1_3_51243406_51244925_51388915_51391864_FF |
| 50 | ATGTTGCTCAGGCTGGTCTCGAGACCAGCCTGACCAACAT | ORF1_8_73772858_73775783_73991731_73996964_RF |
| 51 | CAAACTGAACCGACTCCGTCGACTTCTCCTCTGTAACTGT | ORF1_7_1901148_1902503_2033188_2038434_RF |

TABLE 3.a12

| | PCR Marker Set | qPCR Marker Set |
|---|---|---|
| 1 | OBD168-989.991 | OBD168-q989.q991.p989/p991 |
| 2 | OBD168-2109.2111 | OBD168-q2109.q2111.p2109/p2111 |
| 3 | OBD168-669.671 | OBD168-q669.q671.p669/p671 |
| 4 | OBD168-1729.1731 | OBD168-q1729.q1731.p1729/p1731 |
| 5 | OBD168-701.703 | OBD168-q701.q703.p701/p703 |
| 6 | OBD168-889.891 | OBD168-q889.q891.p889/p891 |
| 7 | OBD168-1141.1143 | OBD168-q1141.q1143.p1141/p1143 |
| 8 | OBD168-1221.1223 | OBD168-q1221.q1223.p1221/p1223 |
| 9 | OBD168-1905.1907 | OBD168-q1905.q1907.p1905/p1907 |
| 10 | OBD168-1465.1467 | OBD168-q1465.q1467.p1465/p1467 |
| 11 | OBD168-1873.1875 | OBD168-q1873.q1875.p1873/p1875 |
| 12 | OBD168-1437.1439 | OBD168-q1437.q1439.p1437/p1439 |
| 13 | OBD168-1841.1843 | OBD168-q1841.q1843.p1841/p1843 |
| 14 | OBD168-905.907 | OBD168-q905.q907.p905/p907 |
| 15 | OBD168-705.707 | OBD168-q705.q707.p705/p707 |
| 16 | OBD168-865.867 | OBD168-q865.q867.p865/p867 |
| 17 | OBD168-1609.1611 | OBD168-q1609.q1611.p1609/p1611 |
| 18 | OBD168-1641.1643 | OBD168-q1641.q1643.p1641/p1643 |
| 19 | OBD168-1061.1063 | OBD168-q1061.q1063.p1061/p1063 |
| 20 | OBD168-2053.2055 | OBD168-q2053.q2055.p2053/p2055 |
| 21 | OBD168-1393.1395 | OBD168-q1393.q1395.p1393/p1395 |
| 22 | OBD168-569.571 | OBD168-q569.q571.p569/p571 |
| 23 | OBD168-1433.1435 | OBD168-q1433.q1435.p1433/p1435 |
| 24 | OBD168-1481.1483 | OBD168-q1481.q1483.p1481/p1483 |
| 25 | OBD168-1953.1955 | OBD168-q1953.q1955.p1953/p1955 |
| 26 | OBD168-1085.1087 | OBD168-q1085.q1087.p1085/p1087 |
| 27 | OBD168-757.759 | OBD168-q757.q759.p757/p759 |
| 28 | OBD168-1965.1967 | OBD168-q1965.q1967.p1965/p1967 |
| 29 | OBD168-697.699 | OBD168-q697.q699.p697/p699 |
| 30 | OBD168-977.979 | OBD168-q977.q979.p977/p979 |
| 31 | OBD168-1117.1119 | OBD168-q1117.q1119.p1117/p1119 |
| 32 | OBD168-1861.1863 | OBD168-q1861.q1863.p1861/p1863 |
| 33 | OBD168-849.851 | OBD168-q849.q851.p849/p851 |
| 34 | OBD168-557.559 | OBD168-q557.q559.p557/p559 |
| 35 | OBD168-765.767 | OBD168-q765.q767.p765/p767 |
| 36 | OBD168-1541.1543 | OBD168-q1541.q1543.p1541/p1543 |
| 37 | OBD168-773.775 | OBD168-q773.q775.p773/p775 |
| 38 | OBD168-1353.1355 | OBD168-q1353.q1355.p1353/p1355 |
| 39 | OBD168-573.575 | OBD168-q573.q575.p573/p575 |
| 40 | OBD168-1489.1491 | OBD168-q1489.q1491.p1489/p1491 |
| 41 | OBD168-737.739 | OBD168-q737.q739.p737/p739 |
| 42 | OBD168-1325.1327 | OBD168-q1325.q1327.p1325/p1327 |
| 43 | OBD168-837.839 | OBD168-q837.q839.p837/p839 |
| 44 | OBD168-805.807 | OBD168-q805.q807.p805/p807 |
| 45 | OBD168-1869.1871 | OBD168-q1869.q1871.p1869/p1871 |
| 46 | OBD168-1981.1983 | OBD168-q1981.q1983.p1981/p1983 |
| 47 | OBD168-1997.1999 | OBD168-q1997.q1999.p1997/p1999 |
| 48 | OBD168-529.531 | OBD168-q529.q531.p529/p531 |
| 49 | OBD168-1605.1607 | OBD168-q1605.q1607.p1605/p1607 |
| 50 | OBD168-2089.2091 | OBD168-q2089.q2091.p2089/p2091 |
| 51 | OBD168-1677.1679 | OBD168-q1677.q1679.p1677/p1679 |

TABLE 3.b1

| | | | |
|---|---|---|---|
| 52 | ORF1_22_41776313_41777375_42031915_42033846_RF | 295 | 1915.600527 |
| 53 | ORF1_2_10016688_10020163_10069588_10070662_RF | 300 | 1950.081799 |
| 54 | ORF1_7_41457346_41461355_41488360_41491403_FF | 230 | 1511.98957 |
| 55 | ORF1_7_135917157_135920025_136070982_136076106_FR | 342 | 2112.969893 |
| 56 | ORF1_6_134102628_134113539_134393136_134395072_RF | 617 | 3237.196304 |
| 57 | ORF1_17_27740779_27745632_27849258_27851150_FR | 179 | 1226.644201 |
| 58 | ORF1_X_39624253_39627672_39748652_39756826_RR | 786 | 3811.777698 |
| 59 | ORF1_21_29066989_29073149_29092829_29094361_RF | 341 | 2111.667632 |
| 60 | ORF1_17_35363623_35366998_35578150_35580887_FF | 427 | 2463.77808 |
| 61 | ORF1_2_129732845_129738418_129813191_129815478_RF | 789 | 3819.174236 |
| 62 | ORF1_15_90688752_90691260_90881362_90884735_RF | 529 | 2871.807678 |
| 63 | ORF1_2_166059673_166070002_166163002_166177651_RF | 228 | 1504.981553 |
| 64 | ORF1_22_23665784_23673822_23711932_23713061_FR | 184 | 1286.837245 |
| 65 | ORF1_1_53182248_53184895_53364674_53371789_RF | 635 | 3294.7368 |
| 66 | ORF1_20_50280026_50281924_50437155_50439548_FF | 373 | 2212.25714 |
| 67 | ORF1_17_20844189_20846843_20895269_20898565_RF | 832 | 3968.695995 |
| 68 | ORF1_19_19361989_19364321_19458777_19462243_RF | 768 | 3761.457058 |
| 69 | ORF1_14_67469788_67473491_67514914_67519335_FR | 478 | 2676.769491 |
| 70 | ORF1_21_29039171_29043861_29092829_29094361_RF | 380 | 2252.095813 |
| 71 | ORF1_19_4252394_4257822_4336234_4339292_FF | 207 | 1414.449942 |
| 72 | ORF1_8_41806799_41808430_41845432_41849175_RF | 232 | 1525.067865 |
| 73 | ORF1_11_47655428_47658417_47713810_47715533_RF | 652 | 3360.825526 |
| 74 | ORF1_1_3073908_3076056_3169997_3177404_FF | 770 | 3771.044431 |
| 75 | ORF1_19_41422837_41424323_41692451_41693876_RR | 766 | 3759.146475 |
| 76 | ORF1_9_134315578_134321630_134391769_134398405_RF | 754 | 3730.532291 |
| 77 | ORF1_3_32625993_32627699_32683326_32685449_FF | 459 | 2572.971635 |
| 78 | ORF1_19_4264058_4265818_4341821_4342968_FR | 221 | 1462.635611 |
| 79 | ORF1_21_29092829_29094361_29164018_29174520_FF | 398 | 2338.87323 |
| 80 | ORF1_2_91859085_91863356_92122473_92125698_RF | 310 | 1980.300976 |
| 81 | ORF1_X_20268599_20270341_20456973_20459740_RR | 811 | 3868.038973 |
| 82 | ORF1_6_29844477_29850988_29921307_29925901_FF | 283 | 1854.11491 |
| 83 | ORF1_19_53278181_53279831_53467555_53470239_FR | 560 | 2987.902851 |
| 84 | ORF1_19_41422837_41424323_41672917_41678014_RR | 249 | 1632.926185 |
| 85 | ORF1_5_690177_692906_744214_749963_FR | 400 | 2340.89338 |
| 86 | ORF1_17_43300561_43304860_43406585_43411982_RF | 846 | 4007.706758 |
| 87 | ORF1_15_62681808_62691633_62730771_62732716_RR | 551 | 2954.724442 |
| 88 | ORF1_12_47686852_47690553_47817317_47827238_RF | 433 | 2485.195218 |
| 89 | ORF1_Y_18283408_18293202_18412555_18418411_RR | 237 | 1545.88146 |
| 90 | ORF1_2_218162078_218164150_218253352_218257050_FF | 787 | 3812.00956 |
| 91 | ORF1_19_41422837_41424323_41597548_41599628_RR | 239 | 1554.462101 |
| 92 | ORF1_20_50280026_50281924_50309417_50313312_FR | 241 | 1558.884246 |
| 93 | ORF1_9_70371691_70373189_70454780_70468182_RR | 727 | 3635.071398 |
| 94 | ORF1_5_75205085_75210191_75236091_75239565_FR | 337 | 2099.567055 |
| 95 | ORF1_X_39735882_39738523_39748652_39756826_FR | 778 | 3797.26634 |
| 96 | ORF1_11_10637008_10641060_10871146_10872290_FR | 248 | 1629.314725 |
| 97 | ORF1_20_63754820_63756674_63899802_63901328_FF | 867 | 4053.634633 |
| 98 | ORF1_4_102249197_102251493_102344820_102348626_FR | 790 | 3820.755115 |
| 99 | ORF1_1_161657362_161661864_161734762_161740896_RF | 644 | 3341.384869 |
| 100 | ORF1_16_8770523_8772703_8938508_8943672_RF | 865 | 4048.304309 |

TABLE 3.b2

| | | | | |
|---|---|---|---|---|
| 52 | 1.446445715 | 0.844353538 | 0.001764866 | Early SSC |
| 53 | 1.444313939 | 0.857296534 | 0.00182229 | Early SSC |
| 54 | 1.444202674 | 0.70719786 | 0.001152482 | Early SSC |
| 55 | 1.442909876 | 0.868305311 | 0.002104088 | Early SSC |
| 56 | 1.44164787 | 1.028118327 | 0.004494626 | Early SSC |
| 57 | 1.441256048 | 0.622029308 | 0.000788913 | Early SSC |
| 58 | 1.439937087 | 1.076426753 | 0.005994767 | Early SSC |
| 59 | 1.435176841 | 0.869890982 | 0.002101767 | Early SSC |
| 60 | 1.434551872 | 0.915034411 | 0.002768411 | Early SSC |
| 61 | 1.434549547 | 1.075994131 | 0.006015229 | Early SSC |
| 62 | 1.434212325 | 0.969982617 | 0.003635674 | Early SSC |
| 63 | 1.432836082 | 0.707437155 | 0.001142847 | Early SSC |
| 64 | 1.431899857 | 0.660136988 | 0.000860631 | Early SSC |
| 65 | 1.430019987 | 1.030563723 | 0.004636752 | Early SSC |
| 66 | 1.428940576 | 0.86431007 | 0.00228425 | Early SSC |
| 67 | 1.422169266 | 1.091590126 | 0.006434995 | Early SSC |
| 68 | 1.419967948 | 1.07621306 | 0.005856319 | Early SSC |
| 69 | 1.418668486 | 0.947418348 | 0.003208743 | Early SSC |
| 70 | 1.417447692 | 0.875891636 | 0.002358301 | Early SSC |
| 71 | 1.416297822 | 0.696494413 | 0.001021535 | Early SSC |
| 72 | 1.415515314 | 0.712096468 | 0.001170556 | Early SSC |
| 73 | 1.414697152 | 1.039518475 | 0.004802253 | Early SSC |
| 74 | 1.41377078 | 1.078233722 | 0.005882594 | Early SSC |
| 75 | 1.411375643 | 1.077857615 | 0.005849994 | Early SSC |
| 76 | 1.409033773 | 1.08039358 | 0.005771898 | Early SSC |
| 77 | 1.40830109 | 0.919601928 | 0.002990734 | Early SSC |
| 78 | 1.407406909 | 0.693140095 | 0.001085372 | Early SSC |
| 79 | 1.40724024 | 0.894701668 | 0.002523054 | Early SSC |
| 80 | 1.406168907 | 0.852847711 | 0.001873262 | Early SSC |
| 81 | 1.405642995 | 1.070456089 | 0.006151131 | Early SSC |
| 82 | 1.404695358 | 0.830066012 | 0.001664425 | Early SSC |
| 83 | 1.403682128 | 0.982988691 | 0.003900334 | Early SSC |
| 84 | 1.40273008 | 0.75057046 | 0.001324208 | Early SSC |
| 85 | 1.400704404 | 0.891601211 | 0.002526946 | Early SSC |
| 86 | 1.400672498 | 1.092114447 | 0.006546419 | Early SSC |
| 87 | 1.39987463 | 0.979467819 | 0.003823905 | Early SSC |
| 88 | 1.399263447 | 0.916381838 | 0.002811445 | Early SSC |
| 89 | 1.398979973 | 0.714351228 | 0.001199569 | Early SSC |
| 90 | 1.398583292 | 1.075173947 | 0.005995408 | Early SSC |
| 91 | 1.397442353 | 0.715489386 | 0.00121162 | Early SSC |
| 92 | 1.397341217 | 0.713200465 | 0.00121785 | Early SSC |
| 93 | 1.395652706 | 1.070547766 | 0.005514495 | Early SSC |
| 94 | 1.395237619 | 0.871205394 | 0.002080251 | Early SSC |
| 95 | 1.39513079 | 1.080227905 | 0.005954705 | Early SSC |
| 96 | 1.394698535 | 0.750594179 | 0.001318931 | Early SSC |
| 97 | 1.394072899 | 1.087179498 | 0.006678603 | Early SSC |
| 98 | 1.392557299 | 1.075414102 | 0.006019606 | Early SSC |
| 99 | 1.391243321 | 1.041707676 | 0.004753319 | Early SSC |
| 100 | 1.389721585 | 1.08718107 | 0.006663206 | Early SSC |

TABLE 3.b3

| | |
|---|---|
| 52 | ATGGGCCTCAGAGGGGAAGAAGTACATATCGAGAATGGGCATCTCTACCCTTGTTAGGTC |
| 53 | ACTGCGCCTGGCATATTTATTTATTTATTCGAGCCGCTTGAGCATAGAGCTTATGGAGAT |
| 54 | TAGTTTCCAAGTGTGTGATAGCTCCAGATCGAGTCTGAGGAGTGAGCGGGAAGCTGCACA |
| 55 | TTTCTCTTTCTCTCTCTCTTTTTTTTTTTCGAGATCCTTAGGGGACCTTGTAGTAAAATA |
| 56 | GACTCCACATTTCCACCAGCTCAAACAGTCGAACAATTCTTCTGCCTCAGCCTCCCAAGC |
| 57 | TGGCTTCTTCTTAACTTTCTCGGGTGAGTCGAAGAGCCCAAATAACTTGTTTTAGACCAG |
| 58 | TGGTTTCAGAAGAGGGGCCCAGGGAACCTCGAATCTTCTGAGGGAGCGTGGCCCAGGTGA |
| 59 | CATCTGTCAGAGTCACCCAGGGGAAGTATCGAAGGTTTTTTGCAGAACAACATACCAAAT |
| 60 | GGCTGAGGTGAGAGAATCACCTGAGCCTTCGAGTTGCAAATTTCTGTTTTCTTTTTTTTT |
| 61 | TGATAGCCAAGACAATGGGGAAAAGGCCTCGACCCTTGCACGGGCGCAGATCCAGGTGCA |
| 62 | AGGCTTCAGCTGGGGCAGGCTTGGCCTGTCGACACCAATGTCTTGGATGTTTAACCTCCA |
| 63 | CATCATGGCAGATGGGTTATCCAACTCCTCGAAAGCTTTCTTTTCTAAGCCTTAGGATGC |
| 64 | AGGTTTCACCATGTTGGCCAGGATGGTCTCGAGATCAGCCTGGTCAACATGGTGAAACCC |
| 65 | CCACCATGCCAGGCACGAACATGGTCACTCGAAATTACGTGAGCCTTAGTTCTTTTTATT |
| 66 | CACACCACCGTTGGGATGAAAGATGGCCTCGATACATGCTTGTCAGTGAAAGAGCGATGC |
| 67 | TCATTGGTAGGACATCTCAAAATGGCACTCGAGCTCTACAGCCAGTACCTGGAGCGCCTG |
| 68 | TGTGCTGGGGGCACTAGTCCCGCTTGCTTCGATGCCTGGGCTCCTTCTGCCCCATCCCCA |
| 69 | GCTGGGCTCCCCGGGCTCAGCGTCCTTGTCGAAACCGAAAGCAAAGAGGAAGCGGGATCT |
| 70 | CATCTGTCAGAGTCACCCAGGGGAAGTATCGATTGTGCTTTCAGTGCCTCAGTGAGATGC |
| 71 | GGGTTTCACCATGTTGGCCAGGCTGGTCTCGAGACCAGCCTGGGCCAACATGGGGAAACC |
| 72 | GGGCTCACTTCATATCCTCTGCCCATCTTCGATGGTTTTCCATGACGATTGTCCAGTTTA |
| 73 | CTCATACATCACAGCCCCAGCCTCGGAATCGACCTCAGCTTCATTCTTCTTAAACAAGAG |
| 74 | ACCAGGAGCAGCCTCTGCTCTGCCCACCTCGAAGCTTCTCAAACGCCACTCTAACGACCC |
| 75 | TGATAAGGCCATGTTTTGGAGACAGTGATCGAGCATTTCTTAAATTCCCTCAGAATTTGC |
| 76 | CGGGGCCCTTTGCTGTGAGTAGAGGATGTCGAGGGCTGGGCGGCAGCCTCCCTGGGCTCC |
| 77 | GCTCTGACATACCACCCTGCCAAGTACCTCGACTCGGGTTCTTCCCTGCAGGTGGCGTGG |
| 78 | GGGTTTCACCATGTTGACCAGGCTGGTCTCGAGACCAGCCTGGCAAACATGGTGAAACCC |
| 79 | CATCTGTCAGAGTCACCCAGGGGAAGTATCGACTAGACACTTCCAAAATACTAACCATAC |
| 80 | GATATTTGTCTAGCTTTGAGGATTTCGTTCGAATTTTAAGCTCCACAAATGACCAAGAAC |
| 81 | CCCTAAATGGCCACGAGGTTCCCCGGCCTCGAGGTGTCAGATATTTTTCTTCCTGTATAT |
| 82 | ACTCTTTCCATTATGTGCAATTGTAACTTCGATGCCAGAGAGGGAGCTCGCCCTGGGAAT |
| 83 | GAGGCAGGTGGAATGGGTGGAGTCCAAGTCGACTCCAAAACCCTAAAACAGGCGCTGGAG |
| 84 | TGATAAGGCCATGTTTTGGAGACAGTGATCGAAGTTTGTGCTCTTTGTGATTCAGACAGA |
| 85 | CCCCTAGGCCTGGGAATAGGGAAACGGTTCGATTTGGTTTCCCAAGGTCTTATCAGTGAT |
| 86 | TTGAGGGCATTTGGCTGCTCTTTTACACTCGAGCCAGCGAAAAGACAGAACCGGAAGAGA |
| 87 | AGACTGGGTGACAGAGTGAGATCGTGTCTCGATTTTCCAGCAGTGTGCCTGGAGGAGTTT |
| 88 | GAGTGGAGAAAACGGTTGGATGAGGGACTCGAGTATAGCATTCAATTGCTCTTTCATATT |
| 89 | TAGGGTCTTCTTCCAGAGAATCAAATTCTCGACCAGCTTGGCCCCATCTAAAGTATGATT |
| 90 | TTTCCTTCTGAGAAGAGATATTCCTTCATCGACTGGTCACGATGGGCTGCTTCATCTGTG |
| 91 | TGATAAGGCCATGTTTTGGAGACAGTGATCGACAGATCTCCATTATTTTTCAGCCGGCTC |
| 92 | CACACCACCGTTGGGATGAAAGATGGCCTCGAGCTGGTCATGACACGCTTTAAGACCCTG |
| 93 | GGGTTTCACCAGGTTGGCCATGCTGGGTTCGAGAGCAGCCTGGGCAACACAGTGAGACCT |
| 94 | TCTTGCTTTCTTGCTTTCTTTCTTGCTTTCGATAACCTGAAATACCGACTTTTTTGGATT |
| 95 | TGATGTTATGGAAAACCATTTGGCCTTTTCGAATCTTCTGAGGGAGCGTGGCCCAGGTGA |
| 96 | GGACATAACTGTATTCTCTCTCTCTCTCTCGAACTCCTGACTTCGGCTGATCTGCCCACC |
| 97 | AAACAGGCACTGCGTGTTCCCAGCACCCTCGATTCCATCTTCCGACCTGCCAGCATCATG |
| 98 | CTCTTTTCTCTTGCAGAGGTGGCAAAACTCGAAAGAACAGCAGCTCGCGACCTGCGGGGC |
| 99 | GTTATTATTCTTCCATTTATATTTTTGTTCGACCCTTCTGCTTTCTCTCCAGGGGATGGC |
| 100 | GTACTGAAGGTCTAGCATATGGGTAGAGTCGACCTCTCTGGGCTCAGGTGATCCTCCCAC |

TABLE 3.b4

| | Probe Location | | | | |
|---|---|---|---|---|---|
| 52 | 22 | 41776315 | 41776344 | 42033815 | 42033844 |
| 53 | 2 | 10016690 | 10016719 | 10070631 | 10070660 |
| 54 | 7 | 41461324 | 41461353 | 41491372 | 41491401 |
| 55 | 7 | 135919994 | 135920023 | 136070984 | 136071013 |
| 56 | 6 | 134102630 | 134102659 | 134395041 | 134395070 |
| 57 | 17 | 27745601 | 27745630 | 27849260 | 27849289 |
| 58 | X | 39624255 | 39624284 | 39748654 | 39748683 |
| 59 | 21 | 29066991 | 29067020 | 29094330 | 29094359 |
| 60 | 17 | 35366967 | 35366996 | 35580856 | 35580885 |
| 61 | 2 | 129732847 | 129732876 | 129815447 | 129815476 |
| 62 | 15 | 90688754 | 90688783 | 90884704 | 90884733 |
| 63 | 2 | 166059675 | 166059704 | 166177620 | 166177649 |
| 64 | 22 | 23673791 | 23673820 | 23711934 | 23711963 |
| 65 | 1 | 53182250 | 53182279 | 53371758 | 53371787 |
| 66 | 20 | 50281893 | 50281922 | 50439517 | 50439546 |
| 67 | 17 | 20844191 | 20844220 | 20898534 | 20898563 |
| 68 | 19 | 19361991 | 19362020 | 19462212 | 19462241 |
| 69 | 14 | 67473460 | 67473489 | 67514916 | 67514945 |
| 70 | 21 | 29039173 | 29039202 | 29094330 | 29094359 |
| 71 | 19 | 4257791 | 4257820 | 4339261 | 4339290 |
| 72 | 8 | 41806801 | 41806830 | 41849144 | 41849173 |
| 73 | 11 | 47655430 | 47655459 | 47715502 | 47715531 |
| 74 | 1 | 3076025 | 3076054 | 3177373 | 3177402 |
| 75 | 19 | 41422839 | 41422868 | 41692453 | 41692482 |
| 76 | 9 | 134315580 | 134315609 | 134398374 | 134398403 |
| 77 | 3 | 32627668 | 32627697 | 32685418 | 32685447 |
| 78 | 19 | 4265787 | 4265816 | 4341823 | 4341852 |
| 79 | 21 | 29094330 | 29094359 | 29174489 | 29174518 |
| 80 | 2 | 91859087 | 91859116 | 92125667 | 92125696 |
| 81 | X | 20268601 | 20268630 | 20456975 | 20457004 |
| 82 | 6 | 29850957 | 29850986 | 29925870 | 29925899 |
| 83 | 19 | 53279800 | 53279829 | 53467557 | 53467586 |
| 84 | 19 | 41422839 | 41422868 | 41672919 | 41672948 |
| 85 | 5 | 692875 | 692904 | 744216 | 744245 |
| 86 | 17 | 43300563 | 43300592 | 43411951 | 43411980 |
| 87 | 15 | 62681810 | 62681839 | 62730773 | 62730802 |
| 88 | 12 | 47686854 | 47686883 | 47827207 | 47827236 |
| 89 | Y | 18283410 | 18283439 | 18412557 | 18412586 |
| 90 | 2 | 218164119 | 218164148 | 218257019 | 218257048 |
| 91 | 19 | 41422839 | 41422868 | 41597550 | 41597579 |
| 92 | 20 | 50281893 | 50281922 | 50309419 | 50309448 |
| 93 | 9 | 70371693 | 70371722 | 70454782 | 70454811 |
| 94 | 5 | 75210160 | 75210189 | 75236093 | 75236122 |
| 95 | X | 39738492 | 39738521 | 39748654 | 39748683 |
| 96 | 11 | 10641029 | 10641058 | 10871148 | 10871177 |
| 97 | 20 | 63756643 | 63756672 | 63901297 | 63901326 |
| 98 | 4 | 102251462 | 102251491 | 102344822 | 102344851 |
| 99 | 1 | 161657364 | 161657393 | 161740865 | 161740894 |
| 100 | 16 | 8770525 | 8770554 | 8943641 | 8943670 |

TABLE 3.b5

| | 4 kb Sequence Location | | | | |
|---|---|---|---|---|---|
| 52 | 22 | 41776315 | 41780314 | 42029845 | 42033844 |
| 53 | 2 | 10016690 | 10020689 | 10066661 | 10070660 |
| 54 | 7 | 41457354 | 41461353 | 41487402 | 41491401 |
| 55 | 7 | 135916024 | 135920023 | 136070984 | 136074983 |
| 56 | 6 | 134102630 | 134106629 | 134391071 | 134395070 |
| 57 | 17 | 27741631 | 27745630 | 27849260 | 27853259 |
| 58 | X | 39624255 | 39628254 | 39748654 | 39752653 |
| 59 | 21 | 29066991 | 29070990 | 29090360 | 29094359 |
| 60 | 17 | 35362997 | 35366996 | 35576886 | 35580885 |
| 61 | 2 | 129732847 | 129736846 | 129811477 | 129815476 |
| 62 | 15 | 90688754 | 90692753 | 90880734 | 90884733 |
| 63 | 2 | 166059675 | 166063674 | 166173650 | 166177649 |
| 64 | 22 | 23669821 | 23673820 | 23711934 | 23715933 |
| 65 | 1 | 53182250 | 53186249 | 53367788 | 53371787 |
| 66 | 20 | 50277923 | 50281922 | 50435547 | 50439546 |
| 67 | 17 | 20844191 | 20848190 | 20894564 | 20898563 |
| 68 | 19 | 19361991 | 19365990 | 19458242 | 19462241 |
| 69 | 14 | 67469490 | 67473489 | 67514916 | 67518915 |
| 70 | 21 | 29039173 | 29043172 | 29090360 | 29094359 |
| 71 | 19 | 4253821 | 4257820 | 4335291 | 4339290 |
| 72 | 8 | 41806801 | 41810800 | 41845174 | 41849173 |
| 73 | 11 | 47655430 | 47659429 | 47711532 | 47715531 |
| 74 | 1 | 3072055 | 3076054 | 3173403 | 3177402 |
| 75 | 19 | 41422839 | 41426838 | 41692453 | 41696452 |
| 76 | 9 | 134315580 | 134319579 | 134394404 | 134398403 |
| 77 | 3 | 32623698 | 32627697 | 32681448 | 32685447 |
| 78 | 19 | 4261817 | 4265816 | 4341823 | 4345822 |
| 79 | 21 | 29090360 | 29094359 | 29170519 | 29174518 |
| 80 | 2 | 91859087 | 91863086 | 92121697 | 92125696 |
| 81 | X | 20268601 | 20272600 | 20456975 | 20460974 |
| 82 | 6 | 29846987 | 29850986 | 29921900 | 29925899 |
| 83 | 19 | 53275830 | 53279829 | 53467557 | 53471556 |
| 84 | 19 | 41422839 | 41426838 | 41672919 | 41676918 |
| 85 | 5 | 688905 | 692904 | 744216 | 748215 |
| 86 | 17 | 43300563 | 43304562 | 43407981 | 43411980 |
| 87 | 15 | 62681810 | 62685809 | 62730773 | 62734772 |
| 88 | 12 | 47686854 | 47690853 | 47823237 | 47827236 |
| 89 | Y | 18283410 | 18287409 | 18412557 | 18416556 |
| 90 | 2 | 218160149 | 218164148 | 218253049 | 218257048 |
| 91 | 19 | 41422839 | 41426838 | 41597550 | 41601549 |
| 92 | 20 | 50277923 | 50281922 | 50309419 | 50313418 |
| 93 | 9 | 70371693 | 70375692 | 70454782 | 70458781 |
| 94 | 5 | 75206190 | 75210189 | 75236093 | 75240092 |
| 95 | X | 39734522 | 39738521 | 39748654 | 39752653 |
| 96 | 11 | 10637059 | 10641058 | 10871148 | 10875147 |
| 97 | 20 | 63752673 | 63756672 | 63897327 | 63901326 |
| 98 | 4 | 102247492 | 102251491 | 102344822 | 102348821 |
| 99 | 1 | 161657364 | 161661363 | 161736895 | 161740894 |
| 100 | 16 | 8770525 | 8774524 | 8939671 | 8943670 |

TABLE 3.b6

| | | |
|---|---|---|
| 52 | ORF1_22_41776313_41777375_42031915_42033846_RF | OBD168-1265 |
| 53 | ORF1_2_10016688_10020163_10069588_10070662_RF | OBD168-1193 |
| 54 | ORF1_7_41457346_41461355_41488360_41491403_FF | OBD168-853 |
| 55 | ORF1_7_135917157_135920025_136070982_136076106_FR | OBD168-1689 |
| 56 | ORF1_6_134102628_134113539_134393136_134395072_RF | OBD168-1089 |
| 57 | ORF1_17_27740779_27745632_27849258_27851150_FR | OBD168-625 |
| 58 | ORF1_X_39624253_39627672_39748652_39756826_RR | OBD168-2101 |
| 59 | ORF1_21_29066989_29073149_29092829_29094361_RF | OBD168-821 |
| 60 | ORF1_17_35363623_35366998_35578150_35580887_FF | OBD168-1865 |
| 61 | ORF1_2_129732845_129738418_129813191_129815478_RF | OBD168-945 |
| 62 | ORF1_15_90688752_90691260_90881362_90884735_RF | OBD168-1845 |
| 63 | ORF1_2_166059673_166070002_166163002_166177651_RF | OBD168-1569 |
| 64 | ORF1_22_23665784_23673822_23711932_23713061_FR | OBD168-1589 |
| 65 | ORF1_1_53182248_53184895_53364674_53371789_RF | OBD168-1149 |
| 66 | ORF1_20_50280026_50281924_50437155_50439548_FF | OBD168-1169 |
| 67 | ORF1_17_20844189_20846843_20895269_20898565_RF | OBD168-473 |
| 68 | ORF1_19_19361989_19364321_19458777_19462243_RF | OBD168-833 |
| 69 | ORF1_14_67469788_67473491_67514914_67519335_FR | OBD168-1829 |
| 70 | ORF1_21_29039171_29043861_29092829_29094361_RF | OBD168-477 |
| 71 | ORF1_19_4252394_4257822_4336234_4339292_FF | OBD168-1877 |
| 72 | ORF1_8_41806799_41808430_41845432_41849175_RF | OBD168-665 |
| 73 | ORF1_11_47655428_47658417_47713810_47715533_RF | OBD168-1765 |
| 74 | ORF1_1_3073908_3076056_3169997_3177404_FF | OBD168-1045 |
| 75 | ORF1_19_41422837_41424323_41692451_41693876_RR | OBD168-761 |
| 76 | ORF1_9_134315578_134321630_134391769_134398405_RF | OBD168-1197 |
| 77 | ORF1_3_32625993_32627699_32683326_32685449_FF | OBD168-1977 |
| 78 | ORF1_19_4264058_4265818_4341821_4342968_FR | OBD168-1881 |
| 79 | ORF1_21_29092829_29094361_29164018_29174520_FF | OBD168-613 |
| 80 | ORF1_2_91859085_91863356_92122473_92125698_RF | OBD168-1917 |
| 81 | ORF1_X_20268599_20270341_20456973_20459740_RR | OBD168-1713 |
| 82 | ORF1_6_29844477_29850988_29921307_29925901_FF | OBD168-1657 |
| 83 | ORF1_19_53278181_53279831_53467555_53470239_FR | OBD168-1897 |
| 84 | ORF1_19_41422837_41424323_41672917_41678014_RR | OBD168-1497 |
| 85 | ORF1_5_690177_692906_744214_749963_FR | OBD168-1109 |
| 86 | ORF1_17_43300561_43304860_43406585_43411982_RF | OBD168-1441 |
| 87 | ORF1_15_62681808_62691633_62730771_62732716_RR | OBD168-817 |
| 88 | ORF1_12_47686852_47690553_47817317_47827238_RF | OBD168-1801 |
| 89 | ORF1_Y_18283408_18293202_18412555_18418411_RR | OBD168-461 |
| 90 | ORF1_2_218162078_218164150_218253352_218257050_FF | OBD168-1577 |
| 91 | ORF1_19_41422837_41424323_41597548_41599628_RR | OBD168-1205 |
| 92 | ORF1_20_50280026_50281924_50309417_50313312_FR | OBD168-1217 |
| 93 | ORF1_9_70371691_70373189_70454780_70468182_RR | OBD168-2093 |
| 94 | ORF1_5_75205085_75210191_75236091_75239565_FR | OBD168-861 |
| 95 | ORF1_X_39735882_39738523_39748652_39756826_FR | OBD168-2105 |
| 96 | ORF1_11_10637008_10641060_10871146_10872290_FR | OBD168-709 |
| 97 | ORF1_20_63754820_63756674_63899802_63901328_FF | OBD168-1229 |
| 98 | ORF1_4_102249197_102251493_102344820_102348626_FR | OBD168-693 |
| 99 | ORF1_1_161657362_161661864_161734762_161740896_RF | OBD168-1053 |
| 100 | ORF1_16_8770523_8772703_8938508_8943672_RF | OBD168-1849 |

TABLE 3.b7

| | | | |
|---|---|---|---|
| 52 | TGTGCTGATGTCTGCTGCTCTCAACA | OBD168-1267 | GATAAAACCCTGATTCTCTCTCCTGG |
| 53 | CCCGTGCTGAGATGGAGAACCTG | OBD168-1195 | GGTTTCACTCCTCCCACCTCATC |
| 54 | GGAGAGTTTCTGTTCAAAGGGTGTAG | OBD168-855 | CCCACAAAGGGTGTTCAATCATCCAC |
| 55 | AGGAAATAAGCCCTACGTGGCAGGAG | OBD168-1691 | CAGGTTTCTCACAAACAGACTGAAAA |
| 56 | GGGAGGGAGAGATTGGTTCTTTACC | OBD168-1091 | ACATAGGTGATGGTGGCTGACTTCCT |
| 57 | AGGAGGGCAAAGGTCACAGTGGC | OBD168-627 | CACCTTGCTGCTTGCCAACAGTG |
| 58 | ATTTAGCAAGACATTGGAAGAA | OBD168-2103 | TTGTGCCTCCGTATACTATAAT |
| 59 | CAGCAGTGGGTTCTCAAATGTTAGCA | OBD168-823 | CTGTCATCTAAGCCTCCATTCATAAC |
| 60 | CAGGCAGATCACTTGAGCCTAG | OBD168-1867 | AAATACAAAAATTAGCTTGGAA |
| 61 | AGCCTGGTCAAGTGAAAAGCCCATTT | OBD168-947 | TGAGGATGACCCAGCAGATAGGGAGA |
| 62 | AGCGGTGGGAGCTGGGGGCGCG | OBD168-1847 | AAGGAGTACTAGTAGATACAAG |
| 63 | ACATACTTTCTAGATCAATTTCTTA | OBD168-1571 | TTTCAAAAACCAACTCCATTAGCAG |
| 64 | TGCAACATCTACCTCCCGGGTTC | OBD168-1591 | GAAACCTCCGCCTCCCAGGTTCA |
| 65 | CCCCAGCCCTCCTTCTCTTCTTT | OBD168-1151 | AATCCCAGCACTCACCACACGGC |
| 66 | GCTTGAAGGCGGATTCCTCCAGG | OBD168-1171 | GCTGCCTGACTACTCCTGGGAAA |
| 67 | GGCTGTGAGGTTGTCCTTTACTGC | OBD168-475 | TCCTGGGAGATGAAGCGACGCAG |
| 68 | TGGGTCTGTGCTTGCTGTGCGGT | OBD168-835 | CCTGCCTGTCTGTCTGGAGAAGA |
| 69 | TGGGGCAACCTCACCAGCTTGGT | OBD168-1831 | AGGCCTTTCCGCCGCCCAGAGAG |
| 70 | CAGCAGTGGGTTCTCAAATGTTAGCA | OBD168-479 | GCTGTCTTGCTGCTGATAGCCTTTA |
| 71 | AGTGATCTGTCTGCCTCGGCCT | OBD168-1879 | GTAAAGTGGTATGATCTTGGCT |
| 72 | GGGACCTTTGAACCACATTTGGAGGG | OBD168-667 | CAGGGATTTCTGAGGACAAGACATCT |
| 73 | TCGGGCGCTTCCGGGGAGCTAGGCC | OBD168-1767 | GGGCACCAAAATGATTAAGGACATT |
| 74 | TGACCAAGAGTGAGCCAAGCCCC | OBD168-1047 | GCCCTTACTGACTGCCAGGAAAC |
| 75 | CTGAACTGTCGTCACTGGTGCTGTG | OBD168-763 | CCTACAAGCCAGAAGGGATTGAGAAT |
| 76 | CTGGAAGAGACAGAGCCCACACA | OBD168-1199 | CGCAGCCCTTCCAAGCCTCAGAA |
| 77 | AAACTAGGAAAAGAGAAATAGAACA | OBD168-1979 | GCTGTGGCGGTCCCTTGGTGGGGAA |
| 78 | CAGCCTGTAGCAATTGTCCTG | OBD168-1883 | AGGTTCAAGCGATTCTCCTGC |
| 79 | CAGCAGTGGGTTCTCAAATGTTAGCA | OBD168-615 | GTCCTCTCTACAAGACAATAGGCAGC |
| 80 | TAGATAGAAGCAATGTCAGAAAC | OBD168-1919 | ATATCGTTTTCCTGAGTAATAGT |
| 81 | AGTCCAAAGCTTCGGGAGAAG | OBD168-1715 | GTCAGGAAAAGAGAGGTAAAT |
| 82 | TAGAGAAGAGAGGACTCTCCCTA | OBD168-1659 | CCGATTAGATACCTGATCATTTA |
| 83 | TCCACAGGGAAAGAGAATCCCTCG | OBD168-1899 | CCTGACGAAGCAGGGGCGGGGCGA |
| 84 | GGAGGGAGGATGGGGGATGAA | OBD168-1499 | AAGTAGCATGCACAGGTTCCA |
| 85 | TTCATTCCAGCCCTCCACGCCCTAAT | OBD168-1111 | GCCAATACATTCAGGAAACACAGGCA |
| 86 | GAGTGCTGAAACTTCCTTTTCTTCT | OBD168-1443 | GGGCATGGGCCCCTCGTCCCCTCGT |
| 87 | GACTGAGCAACATAAGGAGACCCGTC | OBD168-819 | GAGAAAGGAAATGGGACAGAAGCAAT |
| 88 | GACTCTCAGGCACAGAGGCAAAAAA | OBD168-1803 | AACAGATTTTAAAACGTGTCTCATG |
| 89 | AGTGTGTGCCCAGGTGAGAGACCATT | OBD168-463 | GGCAGACGATACATTTCCAGATAGTT |
| 90 | AGGTTGGTGTGGCCCCTGAAGACA | OBD168-1579 | CTGTCCCAGAAAAAAGAAAAATCC |
| 91 | GGGAAGGAGGAGTGGAAACGGAA | OBD168-1207 | AAGCGGAGCCTGGAGCATCTCGT |
| 92 | CTTGAAGGCGGATTCCTCCAGGT | OBD168-1219 | AGCAGGCACGGAACACAGTGGTG |
| 93 | AGCTCACTGCAACCTCTGCCTCCCC | OBD168-2095 | GTTCTGCTGGGGGCAGGCCAGGGGA |
| 94 | GGTGCCACTGCCTTGGTTCCTGT | OBD168-863 | GGTCCCTGCCAAACTGGAAACGA |
| 95 | CACAGGTTGAAAGTGAAAATT | OBD168-2107 | GGTGCTGTACCAAAGTACCAC |
| 96 | GGATAAAGCACAGGATGCCCCAGTTA | OBD168-711 | CGGAAGTCCTACCCAAGCAATCAT |
| 97 | GCTTCCTGAGTCTTCATCACAAAACC | OBD168-1231 | GCCACTGTGCTCAATCTGCGTTCTGA |
| 98 | CCAGGTTCAGACACAGTGCCCAC | OBD168-695 | TCGGCTACGCTGCCACTTCAATG |
| 99 | CAGACTATGTGCCTCAAAGACAGTGC | OBD168-1055 | CAGAAGGGTGAGGACAAAGAGATAGC |
| 100 | GCCATGAGAAACACAAACGACT | OBD168-1851 | GGTGGCTCACACCTGTAATCCC |

TABLE 3.b8

| | | | |
|---|---|---|---|
| 52 | OBD168-q1265 | CTTCCTGTCATCTCTGTGGCTCTCAGCA | OBD168-q1267 |
| 53 | OBD168-q1193 | CGGTCCCCGTGCTGAGATGGAGAACCTG | OBD168-q1195 |
| 54 | OBD168-q853 | GGAGAGTTTCTGTTCAAAGGGTGTAGAG | OBD168-q855 |
| 55 | OBD168-q1689 | CCCCTCTCCTACTAGGAAATAAG | OBD168-q1691 |
| 56 | OBD168-q1089 | GTGATGATGGTTCTCCTGCTCCGTGTCC | OBD168-q1091 |
| 57 | OBD168-q625 | GGCTGCCCCACGGCTACAGAAGGC | OBD168-q627 |
| 58 | OBD168-q2101 | CCCAAAGGCATTTTGGAGAAT | OBD168-q2103 |
| 59 | OBD168-q821 | GACAGCAGTGGGTTCTCAAATGTTAGCA | OBD168-q823 |
| 60 | OBD168-q1865 | GAGCCTAGGAGTTTGAGACGAGT | OBD168-q1867 |
| 61 | OBD168-q945 | GCAGCAAAGCATTCAAGAGTTACCTGGC | OBD168-q947 |
| 62 | OBD168-q1845 | CAGGCGTTCCGAGGGCCAGAG | OBD168-q1847 |
| 63 | OBD168-q1569 | TGTGAGGACATAGTAAGTATA | OBD168-q1571 |
| 64 | OBD168-q1589 | CCCGGGTTCAGGTGATTCTCC | OBD168-q1591 |
| 65 | OBD168-q1149 | CCCCTGCCCTTCTGCCTGGTCTC | OBD168-q1151 |
| 66 | OBD168-q1169 | GCTTGCTTGAAGGCGGATTCCTCCAGG | OBD168-q1171 |
| 67 | OBD168-q473 | AGGCTGTGAGGTTGTCCTTTACTGC | OBD168-q475 |
| 68 | OBD168-q833 | TCCTGGGTCTGTGCTTGCTGTGCGGTGG | OBD168-q835 |
| 69 | OBD168-q1829 | ACCTCACCAGCTTGGTCTCTTTGT | OBD168-q1831 |
| 70 | OBD168-q477 | GACAGCAGTGGGTTCTCAAATGTTAGCA | OBD168-q479 |
| 71 | OBD168-q1877 | GACCTCAAGTGATCTGTCTGCCTCGG | OBD168-q1879 |
| 72 | OBD168-q665 | CATTGCCCCTGGTGCGACCTGCCCC | OBD168-q667 |
| 73 | OBD168-q1765 | GCTTCCGGGGAGCTAGGCCTC | OBD168-q1767 |
| 74 | OBD168-q1045 | TGGGTGCGGCGACAGGCTCATCTGCGAT | OBD168-q1047 |
| 75 | OBD168-q761 | CGTCACTGGTGCTGTGGTGCCCG | OBD168-q763 |
| 76 | OBD168-q1197 | ACCTGGAAGAGACAGAGCCCACACACGG | OBD168-q1199 |
| 77 | OBD168-q1977 | AGTGTCTCACAAAACTCAAGGAAA | OBD168-q1979 |
| 78 | OBD168-q1881 | GTCCTGCCTTAGCCTCCTGAGTAG | OBD168-q1883 |
| 79 | OBD168-q613 | GACAGCAGTGGGTTCTCAAATGTTAGCA | OBD168-q615 |
| 80 | OBD168-q1917 | AGGACTACGGTGAAAAAGGAA | OBD168-q1919 |
| 81 | OBD168-q1713 | AGCGGTCCCCCTGAGTCCAAAGCTT | OBD168-q1715 |
| 82 | OBD168-q1657 | GAAATGGTGCTTTGGTGCTTAGAT | OBD168-q1659 |
| 83 | OBD168-q1897 | GTCTCATTTTCTACCCTCCACA | OBD168-q1899 |
| 84 | OBD168-q1497 | GGGCCCCAGGTCAGTGTGGGCTC | OBD168-q1499 |
| 85 | OBD168-q1109 | GCGGTCTGACAGCCCCACCTGCC | OBD168-q1111 |
| 86 | OBD168-q1441 | GGGGCTGGGAGAGAGGAAGAGATTAT | OBD168-q1443 |
| 87 | OBD168-q817 | GCCAGGGAGTTGGGAAGTGTGGGAAG | OBD168-q819 |
| 88 | OBD168-q1801 | ATTTGTTGAGCATCTACTCTTTG | OBD168-q1803 |
| 89 | OBD168-q461 | AGTGTGTGCCCAGGTGAGAGACCATT | OBD168-q463 |
| 90 | OBD168-q1577 | GGAGGTACCTCAACAGCTCCTCAG | OBD168-q1579 |
| 91 | OBD168-q1205 | CGTCACTGGTGCTGTGGTGCCCG | OBD168-q1207 |
| 92 | OBD168-q1217 | GCTTGCTTGAAGGCGGATTCCTCCAGG | OBD168-q1219 |
| 93 | OBD168-q2093 | GATGGAGTCTCACTCTGTCAC | OBD168-q2095 |
| 94 | OBD168-q861 | CTTTGGTGCCACTGCCTTGGTTCCTGTT | OBD168-q863 |
| 95 | OBD168-q2105 | AAAAAACACTTCCAAAGCTTTTCACC | OBD168-q2107 |
| 96 | OBD168-q709 | CACACGCATCCCTGCTCCCATTCTGAG | OBD168-q711 |
| 97 | OBD168-q1229 | GCAGTTGGCTTCAGACGGGCAGACAGGG | OBD168-q1231 |
| 98 | OBD168-q693 | TGTCCAGGAACAGGAAGTTTCAGAGGGC | OBD168-q695 |
| 99 | OBD168-q1053 | CTCAGACTATGTGCCTCAAAGACAGTGC | OBD168-q1055 |
| 100 | OBD168-q1849 | CAAGAGCCAAAGGATCAACACC | OBD168-q1851 |

TABLE 3.b9

| | | |
|---|---|---|
| 52 | CTGCTGCTGCCTCCATCCTCCTCC | OBD168-p1265 |
| 53 | CGGGCAGAGAGCCAGGGTGAAGC | OBD168-p1193 |
| 54 | GCCCCTTCTCCTTCCCACCATTCCC | OBD168-p853 |
| 55 | CTCACAAACAGACTGAAAAAATA | OBD168-p1689 |
| 56 | CTGCTCCTCTGCCTGCCTTGTAGTCTC | OBD168-p1089 |
| 57 | GTTACACCTTGCTGCTTGCCAACAGTG | OBD168-p625 |
| 58 | ATTAGTTTTCCAGGGGTGCTG | OBD168-p2101 |
| 59 | GCTGTCATCTAAGCCTCCATTCATAACT | OBD168-p823 |
| 60 | TAAAAATACAAAAATTAGCTTGG | OBD168-p1865 |
| 61 | ACTTGGTGAGGCTGGAGGAGGAGGGC | OBD168-p945 |
| 62 | GGAGTACTAGTAGATACAAGA | OBD168-p1845 |
| 63 | GCAGGTAGGGGTGTGGGAGGG | OBD168-p1569 |
| 64 | TGAGACAGAGTCTTGTGCTGT | OBD168-p1589 |
| 65 | GCCCAAATCCCAGCACTCACCACACGGC | OBD168-p1149 |
| 66 | GCTCCTGCTCATCATCTCCCCAGTGG | OBD168-p1169 |
| 67 | ATCGTCCACAGCCCCTGGGTCGG | OBD168-p473 |
| 68 | GCTGCCTGGTGCTGAACTGGTGGG | OBD168-p833 |
| 69 | GGCTCTGCCCAGCCCCTACCCACG | OBD168-p1829 |
| 70 | GCTCCTGGCTGTCTTGCTGCTGATAGC | OBD168-p479 |
| 71 | ACTCTGTTGCCCAGGCTGGAGTAAAG | OBD168-p1877 |
| 72 | GCAGGCATAACCAGGGATTTCTGAGGAC | OBD168-p665 |
| 73 | CAAAATGATTAAGGACATTAA | OBD168-p1765 |
| 74 | CTCCCACCCATACCTCTTCTGCTGGAC | OBD168-p1045 |
| 75 | GCAAGAAGGACCTCACCGCAACTAT | OBD168-p761 |
| 76 | GCCTCGCAGCCCTTCCAAGCCTCAGAA | OBD168-p1197 |
| 77 | CCCGCCGTCTGCCCACTCCTCTAG | OBD168-p1977 |
| 78 | AAGATCTTGGCTCGCTACAACCTC | OBD168-p1881 |
| 79 | CCTCTCTACAAGACAATAGGCAGCATCT | OBD168-p615 |
| 80 | GTTTTCCTGAGTAATAGTTAC | OBD168-p1917 |
| 81 | GTATTGATGTGTGATGAATAAATTG | OBD168-p1713 |
| 82 | TTTACCGATTAGATACCTGATCAT | OBD168-p1657 |
| 83 | GAAGCAGGGGCGGGGCGAGAGG | OBD168-p1897 |
| 84 | TAGCATGCACAGGTTCCAGGGAT | OBD168-p1497 |
| 85 | TGCCACTATCAACCTGACACCAAAGCCA | OBD168-p1109 |
| 86 | GCGCTCCTGCCCTGGGGCCTCGTCTT | OBD168-p1441 |
| 87 | CCGCTAAAGAGAGGAAAAGTGAACTGGA | OBD168-p817 |
| 88 | TTAACAATATTCTTTCTTGAAAT | OBD168-p1801 |
| 89 | CGATACATTTCCAGATAGTTTTGTGGCA | OBD168-p461 |
| 90 | GAGTGAGACCCTGTCCCAGAAAAA | OBD168-p1577 |
| 91 | CAAGCGGAGCCTGGAGCATCTCGTGAGG | OBD168-p1205 |
| 92 | CCCAGGCTCTCTCCATCGCACCTTCTGC | OBD168-p1217 |
| 93 | GGGGGCAGGCCAGGGGACAGG | OBD168-p2093 |
| 94 | GAAACTGGTCCCTGCCAAACTGGAAACG | OBD168-p861 |
| 95 | CTGGATATTTATACAAGAGACTGGAT | OBD168-p2105 |
| 96 | GGCACAGGAGGAACATACCAAAACATAC | OBD168-p709 |
| 97 | TCGTGTCTCACTGCCGCTTCCACCTC | OBD168-p1229 |
| 98 | CGCTCCCCTCGGCTACGCTGCCAC | OBD168-p693 |
| 99 | GGGTGCCCAGTCTGCCACAACCTGTC | OBD168-p1053 |
| 100 | GTGGCTCACACCTGTAATCCCA | OBD168-p1849 |

TABLE 3.b10

| | | |
|---|---|---|
| 52 | AGTACATATCGAGAATGGGCATCTCTACCCTTGTTAGG | OBD168-p1267 |
| 53 | ATTTATTCGAGCCGCTTGAGCATAGAGCTTATGGAGATG | OBD168-p1195 |
| 54 | ATAGCTCCAGATCGAGTCTGAGGAGTGAGCGG | OBD168-p855 |
| 55 | TCTCTCTCTTTTTTTTTTTCGAGATCCTTAGGGGACCTTG | OBD168-p1691 |
| 56 | TCAAACAGTCGAACAATTCTTCTGCCTCAGCCTCCC | OBD168-p1091 |
| 57 | TGGCTTCTTCTTAACTTTCTCGGGTGAGTCGAAGAGC | OBD168-p627 |
| 58 | AGAGGGGCCCAGGGAACCTCGAATCTTCTGAGGGAGCGTG | OBD168-p2103 |
| 59 | ACCTTCGATACTTCCCCTGGGTGACTCTGACAG | OBD168-p821 |
| 60 | AGAGAATCACCTGAGCCTTCGAGTTGCAAATTTCTGTTTT | OBD168-p1867 |
| 61 | AAAAGGCCTCGACCCTTGCACGGGCGCA | OBD168-p947 |
| 62 | TGGGGCAGGCTTGGCCTGTCGACACCAATGTCTTGGATGT | OBD168-p1847 |
| 63 | GATGGGTTATCCAACTCCTCGAAAGCTTTCTTTTCTAAGC | OBD168-p1571 |
| 64 | ATGTTGGCCAGGATGGTCTCGAGATCAGCCTGGTCAACAT | OBD168-p1591 |
| 65 | TGCCAGGCACGAACATGGTCACTCGAAATTACGTG | OBD168-p1151 |
| 66 | ACACCACCGTTGGGATGAAAGATGGCCTCGATACA | OBD168-p1171 |
| 67 | AATGGCACTCGAGCTCTACAGCCAGTACCTGG | OBD168-p475 |
| 68 | ACTAGTCCCGCTTGCTTCGATGCCTGGGCT | OBD168-p835 |
| 69 | CCGGGCTCAGCGTCCTTGTCGAAACCGAAAGCAAAGAGGA | OBD168-p1831 |
| 70 | AGCACAATCGATACTTCCCCTGGGTGACTCTGAC | OBD168-p477 |
| 71 | ATGTTGGCCAGGCTGGTCTCGAGACCAGCCTGGGCCAACA | OBD168-p1879 |
| 72 | TCCTCTGCCCATCTTCGATGGTTTTCCATGACGATTG | OBD168-p667 |
| 73 | ACAGCCCCAGCCTCGGAATCGACCTCAGCTTCATTCTTCT | OBD168-p1767 |
| 74 | AGCAGCCTCTGCTCTGCCCACCTCGAAGCTT | OBD168-p1047 |
| 75 | AGGCCATGTTTTGGAGACAGTGATCGAGCATTTC | OBD168-p763 |
| 76 | TTTGCTGTGAGTAGAGGATGTCGAGGGCTGGG | OBD168-p1199 |
| 77 | ACCACCCTGCCAAGTACCTCGACTCGGGTTCTTCCCTGCA | OBD168-p1979 |
| 78 | ATGTTGACCAGGCTGGTCTCGAGACCAGCCTGGCAAACAT | OBD168-p1883 |
| 79 | TCTAGTCGATACTTCCCCTGGGTGACTCTGACAG | OBD168-p613 |
| 80 | TAGCTTTGAGGATTTCGTTCGAATTTTAAGCTCCACAAAT | OBD168-p1919 |
| 81 | CCACGAGGTTCCCCGGCCTCGAGGTGTCAGATATTTTTCT | OBD168-p1715 |
| 82 | TTATGTGCAATTGTAACTTCGATGCCAGAGAGGGAGCTCG | OBD168-p1659 |
| 83 | GAATGGGTGGAGTCCAAGTCGACTCCAAAACCCTAAAACA | OBD168-p1899 |
| 84 | ATGTTTTGGAGACAGTGATCGAAGTTTGTGCTCTTTGTGA | OBD168-p1499 |
| 85 | AGGCCTGGGAATAGGGAAACGGTTCGATTTGG | OBD168-p1111 |
| 86 | TTGGCTGCTCTTTTACACTCGAGCCAGCGAAAAGACAGAA | OBD168-p1443 |
| 87 | TCGTGTCTCGATTTTCCAGCAGTGTGCCTGGAG | OBD168-p819 |
| 88 | AACGGTTGGATGAGGGACTCGAGTATAGCATTCAATTGCT | OBD168-p1803 |
| 89 | AGAATCAAATTCTCGACCAGCTTGGCCCCATCTAAAG | OBD168-p463 |
| 90 | AGAAGAGATATTCCTTCATCGACTGGTCACGATGGGCTGC | OBD168-p1579 |
| 91 | AGGCCATGTTTTGGAGACAGTGATCGACAGATCTCC | OBD168-p1207 |
| 92 | ACCGTTGGGATGAAAGATGGCCTCGAGCTGGT | OBD168-p1219 |
| 93 | AGGTTGGCCATGCTGGGTTCGAGAGCAGCCTGGGCAACAC | OBD168-p2095 |
| 94 | TTCTTGCTTTCTTGCTTTCTTTCTTGCTTTCGATAACCTG | OBD168-p863 |
| 95 | GAAAACCATTTGGCCTTTTCGAATCTTCTGAGGGAGCGTG | OBD168-p2107 |
| 96 | TCTCTCTCTCTCTCTCGAACTCCTGACTTCGGC | OBD168-p711 |
| 97 | ACTGCGTGTTCCCAGCACCCTCGATTCCATCT | OBD168-p1231 |
| 98 | AGGTGGCAAAACTCGAAAGAACAGCAGCTCGCG | OBD168-p695 |
| 99 | TTTTGTTCGACCCTTCTGCTTTCTCTCCAGGG | OBD168-p1055 |
| 100 | TCTAGCATATGGGTAGAGTCGACCTCTCTGGGCTCAGGTG | OBD168-p1851 |

TABLE 3.b11

| | | |
|---|---|---|
| 52 | TGCCCATTCTCGATATGTACTTCTTCCCCTCTGAGG | ORF1_22_41776313_41777375_42031915_42033846_RF |
| 53 | AGCGGCTCGAATAAATAAATAAATATGCCAGGCGCAGTG | ORF1_2_10016688_10020163_10069588_10070662_RF |
| 54 | TTCCCGCTCACTCCTCAGACTCGATCTGGAG | ORF1_7_41457346_41461355_41488360_41491403_FF |
| 55 | CAAGGTCCCCTAAGGATCTCGAAAAAAAAAAAGAGAGAGA | ORF1_7_135917157_135920025_136070982_136076106_FR |
| 56 | TGGGAGGCTGAGGCAGAAGAATTGTTCGACTGTTTG | ORF1_6_134102628_134113539_134393136_134395072_RF |
| 57 | TGGGCTCTTCGACTCACCCGAGAAAGTTAAGAAGAAG | ORF1_17_27740779_27745632_27849258_27851150_FR |
| 58 | CACGCTCCCTCAGAAGATTCGAGGTTCCCTGGGCCCCTCT | ORF1_X_39624253_39627672_39748652_39756826_RR |
| 59 | CTGTCAGAGTCACCCAGGGGAAGTATCGAAGGT | ORF1_21_29066989_29073149_29092829_29094361_RF |
| 60 | AAAACAGAAATTTGCAACTCGAAGGCTCAGGTGATTCTCT | ORF1_17_35363623_35366998_35578150_35580887_FF |
| 61 | AGGGTCGAGGCCTTTTCCCCATTGTCTTGGCT | ORF1_2_129732845_129738418_129813191_129815478_RF |
| 62 | ACATCCAAGACATTGGTGTCGACAGGCCAAGCCTGCCCCA | ORF1_15_90688752_90691260_90881362_90884735_RF |
| 63 | GCTTAGAAAAGAAAGCTTTCGAGGAGTTGGATAACCCATC | ORF1_2_166059673_166070002_166163002_166177651_RF |
| 64 | ATGTTGACCAGGCTGATCTCGAGACCATCCTGGCCAACAT | ORF1_22_23665784_23673822_23711932_23713061_FR |
| 65 | TCACGTAATTTCGAGTGACCATGTTCGTGCCTGGC | ORF1_1_53182248_53184895_53364674_53371789_RF |
| 66 | TGTATCGAGGCCATCTTTCATCCCAACGGTGGTG | ORF1_20_50280026_50281924_50437155_50439548_FF |
| 67 | TCCAGGTACTGGCTGTAGAGCTCGAGTGCCAT | ORF1_17_20844189_20846843_20895269_20898565_RF |
| 68 | AGCCCAGGCATCGAAGCAAGCGGGACTAGT | ORF1_19_19361989_19364321_19458777_19462243_RF |
| 69 | TCCTCTTTGCTTTCGGTTTCGACAAGGACGCTGAGCCCGG | ORF1_14_67469788_67473491_67514914_67519335_FR |
| 70 | GTCAGAGTCACCCAGGGGAAGTATCGATTGTGCT | ORF1_21_29039171_29043861_29092829_29094361_RF |
| 71 | TGTTGGCCCAGGCTGGTCTCGAGACCAGCCTGGCCAACAT | ORF1_19_4252394_4257822_4336234_4339292_FF |
| 72 | ACCATCGAAGATGGGCAGAGGATATGAAGTGAGCC | ORF1_8_41806799_41808430_41845432_41849175_RF |
| 73 | AGAAGAATGAAGCTGAGGTCGATTCCGAGGCTGGGGCTGT | ORF1_11_47655428_47658417_47713810_47715533_RF |
| 74 | AAGCTTCGAGGTGGGCAGAGCAGAGGCTG | ORF1_1_3073908_3076056_3169997_3177404_FF |
| 75 | ATGCTCGATCACTGTCTCCAAAACATGGCCTTATCACC | ORF1_19_41422837_41424323_41692451_41693876_RR |
| 76 | AGCCCTCGACATCCTCTACTCACAGCAAAGGG | ORF1_9_134315578_134321630_134391769_134398405_RF |
| 77 | TGCAGGGAAGAACCCGAGTCGAGGTACTTGGCAGGGTGGT | ORF1_3_32625993_32627699_32683326_32685449_FF |
| 78 | ATGTTTGCCAGGCTGGTCTCGAGACCAGCCTGGTCAACAT | ORF1_19_4264058_4265818_4341821_4342968_FR |
| 79 | CTGTCAGAGTCACCCAGGGGAAGTATCGACTAGA | ORF1_21_29092829_29094361_29164018_29174520_FF |
| 80 | ATTTGTGGAGCTTAAAATTCGAACGAAATCCTCAAAGCTA | ORF1_2_91859085_91863356_92122473_92125698_RF |
| 81 | AGAAAAATATCTGACACCTCGAGGCCGGGGAACCTCGTGG | ORF1_X_20268599_20270341_20456973_20459740_RR |
| 82 | CGAGCTCCCTCTCTGGCATCGAAGTTACAATTGCACATAA | ORF1_6_29844477_29850988_29921307_29925901_FF |
| 83 | TGTTTTAGGGTTTTGGAGTCGACTTGGACTCCACCCATTC | ORF1_19_53278181_53279831_53467555_53470239_FR |
| 84 | TCACAAAGAGCACAAACTTCGATCACTGTCTCCAAAACAT | ORF1_19_41422837_41424323_41672917_41678014_RR |
| 85 | ACCAAATCGAACCGTTTCCCTATTCCCAGGCCTAG | ORF1_5_690177_692906_744214_749963_FR |
| 86 | TTCTGTCTTTTCGCTGGCTCGAGTGTAAAAGAGCAGCCAA | ORF1_17_43300561_43304860_43406585_43411982_RF |
| 87 | TCCAGGCACACTGCTGGAAAATCGAGACACGATC | ORF1_15_62681808_62691633_62730771_62732716_RR |
| 88 | AGCAATTGAATGCTATACTCGAGTCCCTCATCCAACCGTT | ORF1_12_47686852_47690553_47817317_47827238_RF |
| 89 | CTTTAGATGGGGCCAAGCTGGTCGAGAATTTGATTCT | ORF1_Y_18283408_18293202_18412555_18418411_RR |
| 90 | GCAGCCCATCGTGACCAGTCGATGAAGGAATATCTCTTCT | ORF1_2_218162078_218164150_218253352_218257050_FF |
| 91 | TCTGTCGATCACTGTCTCCAAAACATGGCCTTATCACC | ORF1_19_41422837_41424323_41597548_41599628_RR |
| 92 | ACCAGCTCGAGGCCATCTTTCATCCCAACGGT | ORF1_20_50280026_50281924_50309417_50313312_FR |
| 93 | GTGTTGCCCAGGCTGCTCTCGAACCCAGCATGGCCAACCT | ORF1_9_70371691_70373189_70454780_70468182_RR |
| 94 | AGGTTATCGAAAGCAAGAAAGAAAGCAAGAAAGCAAGAAG | ORF1_5_75205085_75210191_75236091_75239565_FR |
| 95 | CACGCTCCCTCAGAAGATTCGAAAAGGCCAAATGGTTTTC | ORF1_X_39735882_39738523_39748652_39756826_FR |
| 96 | AGCCGAAGTCAGGAGTTCGAGAGAGAGAGAGAG | ORF1_11_10637008_10641060_10871146_10872290_FR |
| 97 | AGATGGAATCGAGGGTGCTGGGAACACGCAGT | ORF1_20_63754820_63756674_63899802_63901328_FF |
| 98 | TCGCGAGCTGCTGTTCTTTCGAGTTTTGCCACC | ORF1_4_102249197_102251493_102344820_102348626_FR |
| 99 | TCCCCTGGAGAGAAAGCAGAAGGGTCGAACAAAA | ORF1_1_161657362_161661864_161734762_161740896_RF |
| 100 | CACCTGAGCCCAGAGAGGTCGACTCTACCCATATGCTAGA | ORF1_16_8770523_8772703_8938508_8943672_RF |

TABLE 3.b12

| | | |
|---|---|---|
| 52 | OBD168-1265.1267 | OBD168-q1265.q1267.p1265/p1267 |
| 53 | OBD168-1193.1195 | OBD168-q1193.q1195.p1193/p1195 |
| 54 | OBD168-853.855 | OBD168-q853.q855.p853/p855 |
| 55 | OBD168-1689.1691 | OBD168-q1689.q1691.p1689/p1691 |
| 56 | OBD168-1089.1091 | OBD168-q1089.q1091.p1089/p1091 |
| 57 | OBD168-625.627 | OBD168-q625.q627.p625/p627 |
| 58 | OBD168-2101.2103 | OBD168-q2101.q2103.p2101/p2103 |
| 59 | OBD168-821.823 | OBD168-q821.q823.p823/p821 |
| 60 | OBD168-1865.1867 | OBD168-q1865.q1867.p1865/p1867 |
| 61 | OBD168-945.947 | OBD168-q945.q947.p945/p947 |
| 62 | OBD168-1845.1847 | OBD168-q1845.q1847.p1845/p1847 |
| 63 | OBD168-1569.1571 | OBD168-q1569.q1571.p1569/p1571 |
| 64 | OBD168-1589.1591 | OBD168-q1589.q1591.p1589/p1591 |
| 65 | OBD168-1149.1151 | OBD168-q1149.q1151.p1149/p1151 |
| 66 | OBD168-1169.1171 | OBD168-q1169.q1171.p1169/p1171 |
| 67 | OBD168-473.475 | OBD168-q473.q475.p473/p475 |
| 68 | OBD168-833.835 | OBD168-q833.q835.p833/p835 |
| 69 | OBD168-1829.1831 | OBD168-q1829.q1831.p1829/p1831 |
| 70 | OBD168-477.479 | OBD168-q477.q479.p479/p477 |
| 71 | OBD168-1877.1879 | OBD168-q1877.q1879.p1877/p1879 |
| 72 | OBD168-665.667 | OBD168-q665.q667.p665/p667 |
| 73 | OBD168-1765.1767 | OBD168-q1765.q1767.p1765/p1767 |
| 74 | OBD168-1045.1047 | OBD168-q1045.q1047.p1045/p1047 |
| 75 | OBD168-761.763 | OBD168-q761.q763.p761/p763 |
| 76 | OBD168-1197.1199 | OBD168-q1197.q1199.p1197/p1199 |
| 77 | OBD168-1977.1979 | OBD168-q1977.q1979.p1977/p1979 |
| 78 | OBD168-1881.1883 | OBD168-q1881.q1883.p1881/p1883 |
| 79 | OBD168-613.615 | OBD168-q613.q615.p615/p613 |
| 80 | OBD168-1917.1919 | OBD168-q1917.q1919.p1917/p1919 |
| 81 | OBD168-1713.1715 | OBD168-q1713.q1715.p1713/p1715 |
| 82 | OBD168-1657.1659 | OBD168-q1657.q1659.p1657/p1659 |
| 83 | OBD168-1897.1899 | OBD168-q1897.q1899.p1897/p1899 |
| 84 | OBD168-1497.1499 | OBD168-q1497.q1499.p1497/p1499 |
| 85 | OBD168-1109.1111 | OBD168-q1109.q1111.p1109/p1111 |
| 86 | OBD168-1441.1443 | OBD168-q1441.q1443.p1441/p1443 |
| 87 | OBD168-817.819 | OBD168-q817.q819.p817/p819 |
| 88 | OBD168-1801.1803 | OBD168-q1801.q1803.p1801/p1803 |
| 89 | OBD168-461.463 | OBD168-q461.q463.p461/p463 |
| 90 | OBD168-1577.1579 | OBD168-q1577.q1579.p1577/p1579 |
| 91 | OBD168-1205.1207 | OBD168-q1205.q1207.p1205/p1207 |
| 92 | OBD168-1217.1219 | OBD168-q1217.q1219.p1217/p1219 |
| 93 | OBD168-2093.2095 | OBD168-q2093.q2095.p2093/p2095 |
| 94 | OBD168-861.863 | OBD168-q861.q863.p861/p863 |
| 95 | OBD168-2105.2107 | OBD168-q2105.q2107.p2105/p2107 |
| 96 | OBD168-709.711 | OBD168-q709.q711.p709/p711 |
| 97 | OBD168-1229.1231 | OBD168-q1229.q1231.p1229/p1231 |
| 98 | OBD168-693.695 | OBD168-q693.q695.p693/p695 |
| 99 | OBD168-1053.1055 | OBD168-q1053.q1055.p1053/p1055 |
| 100 | OBD168-1849.1851 | OBD168-q1849.q1851.p1849/p1851 |

TABLE 4.a1

| | Probe | gene.index | RP/Rsum |
|---|---|---|---|
| 1 | ORF1_11_61243450_61246026_61388261_61391790_FR | 65 | 683.7987722 |
| 2 | ORF1_3_18693806_18695229_18731498_18743005_FR | 121 | 1311.207619 |
| 3 | ORF1_2_118112112_118113416_118185366_118187110_FR | 42 | 424.4713289 |
| 4 | ORF1_17_27395089_27399400_27471513_27474666_RR | 123 | 1345.935183 |
| 5 | ORF1_12_131862265_131863764_132122269_132123632_RF | 192 | 1895.333542 |
| 6 | ORF1_12_121447890_121451709_121468997_121471919_FF | 64 | 676.3740979 |
| 7 | ORF1_14_81120330_81122262_81268937_81270783_RF | 225 | 2139.947545 |
| 8 | ORF1_13_45338339_45340076_45393094_45398989_FR | 247 | 2241.882572 |
| 9 | ORF1_12_5915876_5919160_6152139_6154132_FF | 95 | 973.1243446 |
| 10 | ORF1_16_9911562_9916434_9954809_9955829_RF | 204 | 2003.48294 |
| 11 | ORF1_22_16907330_16914217_17087415_17089015_RR | 189 | 1885.344261 |
| 12 | ORF1_9_131999122_132006607_132139741_132142158_FR | 216 | 2077.891178 |
| 13 | ORF1_12_5915876_5919160_6059110_6067436_FF | 112 | 1190.76143 |
| 14 | ORF1_19_52960383_52965476_52976109_52979684_FR | 105 | 1105.727185 |
| 15 | ORF1_1_229299065_229300173_229506871_229512794_FR | 224 | 2125.739433 |
| 16 | ORF1_16_18252588_18255195_18400348_18402976_RR | 274 | 2399.456066 |
| 17 | ORF1_2_111366352_111370512_111529947_111533147_FF | 282 | 2454.310373 |
| 18 | ORF1_5_55186470_55190634_55308382_55310405_FR | 119 | 1277.819011 |
| 19 | ORF1_7_2130888_2136507_2376124_2377913_FF | 308 | 2576.79482 |
| 20 | ORF1_12_109959287_109961500_109999941_110004320_FF | 292 | 2522.263056 |
| 21 | ORF1_5_148804534_148807399_148901266_148906110_RF | 315 | 2610.322544 |
| 22 | ORF1_2_174610120_174616390_174648154_174652062_RR | 205 | 2020.977518 |
| 23 | ORF1_17_43832124_43833485_43905109_43906650_FF | 273 | 2395.722643 |
| 24 | ORF1_4_74078231_74079386_74117911_74125141_FR | 284 | 2461.265909 |
| 25 | ORF1_1_36159277_36165949_36303183_36304407_RF | 260 | 2319.480175 |
| 26 | ORF1_5_124631740_124634000_124857558_124858678_FR | 269 | 2362.930803 |
| 27 | ORF1_6_86970655_86977787_87154540_87156275_RR | 335 | 2708.303887 |
| 28 | ORF1_4_38133158_38135017_38161792_38166364_RF | 158 | 1684.416661 |
| 29 | ORF1_1_42817616_42818863_42949613_42952408_FF | 156 | 1670.587608 |
| 30 | ORF1_10_44948033_44949598_44963508_44966433_RR | 215 | 2075.653386 |
| 31 | ORF1_18_75155500_75159840_75214354_75217340_RF | 319 | 2622.075007 |
| 32 | ORF1_1_151547581_151552501_151755108_151762446_RF | 317 | 2616.065515 |
| 33 | ORF1_X_49053496_49056424_49286142_49288605_RF | 214 | 2072.613711 |
| 34 | ORF1_11_61243450_61246026_61472770_61474691_FR | 173 | 1776.408098 |
| 35 | ORF1_14_81530269_81532452_81602546_81604597_FF | 313 | 2604.836014 |
| 36 | ORF1_14_74588744_74593680_74780483_74782393_FF | 374 | 2923.537449 |
| 37 | ORF1_2_27481760_27484387_27626184_27635183_RF | 281 | 2452.218225 |
| 38 | ORF1_18_10287457_10291556_10505933_10507774_FF | 382 | 2969.774836 |
| 39 | ORF1_1_109681178_109683602_109788010_109790308_FF | 352 | 2769.148458 |
| 40 | ORF1_11_95040575_95045594_95230445_95231653_FF | 145 | 1598.706819 |
| 41 | ORF1_6_6745894_6751892_6983650_6986668_FR | 324 | 2654.348688 |
| 42 | ORF1_6_113632952_113639716_113727754_113732296_RF | 345 | 2749.196717 |
| 43 | ORF1_11_61224624_61227200_61388261_61391790_FR | 193 | 1898.912573 |
| 44 | ORF1_10_91056658_91062643_91164372_91168358_RF | 174 | 1780.548073 |
| 45 | ORF1_2_102100064_102105009_102374574_102377537_FR | 207 | 2033.960539 |
| 46 | ORF1_11_17097711_17100441_17188135_17191681_RR | 322 | 2640.403311 |
| 47 | ORF1_1_199677018_199683841_199811400_199820398_RR | 395 | 3048.106352 |
| 48 | ORF1_7_100090606_100091968_100119089_100121546_RR | 410 | 3137.941194 |
| 49 | ORF1_12_7868599_7870934_7932397_7933511_FF | 201 | 1979.138215 |
| 50 | ORF1_7_87727621_87737484_87868535_87872727_FF | 339 | 2722.114145 |

TABLE 4.a2

| | FC: (class1/class2) | pfp | P.value | Type |
|---|---|---|---|---|
| 1 | −1.880110497 | 0.586539786 | 0.000270132 | Late SSc |
| 2 | −1.799608311 | 1.03860777 | 0.000890435 | Late SSc |
| 3 | −1.780583699 | 0.371293764 | 0.000110492 | Late SSc |
| 4 | −1.667670091 | 1.071320657 | 0.000933662 | Late SSc |
| 5 | −1.602310181 | 1.272773307 | 0.00173148 | Late SSc |
| 6 | −1.594316169 | 0.583773394 | 0.000264722 | Late SSc |
| 7 | −1.585703511 | 1.35013943 | 0.002152417 | Late SSc |
| 8 | −1.56216833 | 1.336623574 | 0.002339221 | Late SSc |
| 9 | −1.556271307 | 0.768375125 | 0.000517204 | Late SSc |
| 10 | −1.555096184 | 1.323326956 | 0.001912769 | Late SSc |
| 11 | −1.540705079 | 1.280763297 | 0.001715126 | Late SSc |
| 12 | −1.538681271 | 1.334213022 | 0.002041946 | Late SSc |
| 13 | −1.536705015 | 0.941892109 | 0.000747454 | Late SSc |
| 14 | −1.536449321 | 0.877882311 | 0.000653117 | Late SSc |
| 15 | −1.527220454 | 1.340092929 | 0.002126906 | Late SSc |
| 16 | −1.52149253 | 1.360282508 | 0.002640857 | Late SSc |
| 17 | −1.519473048 | 1.376054571 | 0.002749477 | Late SSc |
| 18 | −1.503923716 | 1.007768197 | 0.000849714 | Late SSc |
| 19 | −1.502514474 | 1.374072688 | 0.002998649 | Late SSc |
| 20 | −1.502130011 | 1.39520081 | 0.002886588 | Late SSc |
| 21 | −1.50156792 | 1.374806784 | 0.003068439 | Late SSc |
| 22 | −1.500042403 | 1.337558365 | 0.001942817 | Late SSc |
| 23 | −1.496149497 | 1.361478188 | 0.002633532 | Late SSc |
| 24 | −1.494024976 | 1.373274177 | 0.002763382 | Late SSc |
| 25 | −1.492254542 | 1.349373491 | 0.002485826 | Late SSc |
| 26 | −1.482938536 | 1.348160178 | 0.002569562 | Late SSc |
| 27 | −1.48249355 | 1.380279235 | 0.00327625 | Late SSc |
| 28 | −1.482383403 | 1.250937648 | 0.001400419 | Late SSc |
| 29 | −1.47881757 | 1.248294422 | 0.001379771 | Late SSc |
| 30 | −1.477514304 | 1.337834051 | 0.002038008 | Late SSc |
| 31 | −1.475489797 | 1.368461872 | 0.003093062 | Late SSc |
| 32 | −1.473695833 | 1.371485397 | 0.003080461 | Late SSc |
| 33 | −1.473125886 | 1.34056185 | 0.002032665 | Late SSc |
| 34 | −1.47310541 | 1.257251246 | 0.001541109 | Late SSc |
| 35 | −1.472213708 | 1.378421017 | 0.003056972 | Late SSc |
| 36 | −1.467917288 | 1.416119903 | 0.00375264 | Late SSc |
| 37 | −1.467652572 | 1.378853794 | 0.0027453 | Late SSc |
| 38 | −1.467315677 | 1.425572936 | 0.003858496 | Late SSc |
| 39 | −1.4669037 | 1.36651033 | 0.003408167 | Late SSc |
| 40 | −1.464113123 | 1.240602908 | 0.001274577 | Late SSc |
| 41 | −1.462599528 | 1.37698371 | 0.003161106 | Late SSc |
| 42 | −1.457734612 | 1.37644207 | 0.003364669 | Late SSc |
| 43 | −1.456367968 | 1.270475422 | 0.001737356 | Late SSc |
| 44 | −1.451682509 | 1.255271302 | 0.001547576 | Late SSc |
| 45 | −1.446541214 | 1.339926641 | 0.001965245 | Late SSc |
| 46 | −1.443313466 | 1.372615939 | 0.003131628 | Late SSc |
| 47 | −1.442937935 | 1.443731983 | 0.004040629 | Late SSc |
| 48 | −1.442059337 | 1.464295228 | 0.004253807 | Late SSc |
| 49 | −1.441214801 | 1.313952351 | 0.001871289 | Late SSc |
| 50 | −1.439270427 | 1.376378475 | 0.003306 | Late SSc |

TABLE 4.a3

| | Probe sequence |
|---|---|
| | 60 mer |
| 1 | CCTGGCTCCTTCCTGTATTATGCTCCCTTCGAGAAGTAGAATATCTGAATGTTCTTATAT |
| 2 | TGGACTGGGCTAATGTGGAAAGTGAGGATCGAGAATGAAGAAGGAACAGTCACGAAGGGA |
| 3 | TATTTATTTTTATTTTTATTTATTTTTTTCGATTGTGTTCACCAGGACGGCAGGCTATTA |
| 4 | CACATGGCTAAAAGGAAGACTGACAATTTCGAGCTTGGTGTGGTCAAGGGCCAGGAGGAG |
| 5 | TAATATTGGCTTTGGCTAATCAGAGATTTCGACAGGAGGATGGTGGGGAGGGGAGGCCAG |
| 6 | GGTGGGTAGATCACTTGAGGTCAGGAGTTCGAACTCCTGACCTCAAGTGATCCACGCCCA |
| 7 | GGAGGCAGAAGTTGTGGGCATGCTGAACTCGAGGAAATCCATTGACATGAAAAACGTCAA |
| 8 | TTCCTGCAGGTGATGGTTCATGACAATATCGACTGAGCTGCCAAGTATTTCATACATGGG |
| 9 | GAGTGTGATGAAGAAAAGTAAGGCAGGATCGATGAATTCTAGGGCATGTAAATGCTCAAG |
| 10 | GCGAATGAGCTCCCTGTGTCACACAGCATCGAGAGTCCTGAGCACCATCCCAATCCCTGT |
| 11 | TCTAGGCTTCAAGAGAGAGAATCCAATGTCGATTTAGCCTTCCATAACTATCCTCAGACA |
| 12 | AATGGTGTCTATAGCCAGGTTTTTGTAATCGAAGGACCCCTTGCTGGGGGCGCCTAAAGA |
| 13 | GAGTGTGATGAAGAAAAGTAAGGCAGGATCGAATATTTGCACATTTGTCGTTGACCCTTC |
| 14 | CTATATTTTTCACTCACATCCCTAGTTATCGAGAACATCCTGGCTAATGTGATGAAACCC |
| 15 | ACGCTGAACTAATTGGGCTGAAGTATGGTCGAACAGGTTACGAGCATCAGCTGTTACATG |
| 16 | CATTGGTCACAATGGGTCACGTGCCTATTCGAGACCACCCAGCAAAGCCCCAAGGACACA |
| 17 | TGCACCTCTCTTTTGAATCCCTCCCCCATCGAGGTTAATCCCTCTCCACAGGGCGGCACC |
| 18 | AGAGAGAGTAACAAATACGAGGAGGGTTTCGATGTTACTGATTCTGTCCCCATCTTCGCG |
| 19 | ACTCTCTTACCCAAGTTTCCTAACAGGATCGACACCCAGGGTCATTCACACAACACGGCT |
| 20 | CTGAAGCCAACACACGCAAAGGCAAACGTCGAAACTGATCATTTAGGTTTTCCTTTTAAG |
| 21 | GTTTACAGACCTTGGATGCAGGCTCTTCTCGAGGGGAAAAAGGAGCAAGTTGGGAGGTGA |
| 22 | CCTGTGGCCTGATAAGGTATTGACCCCATCGAGATTTTATTAATTAGTGGCAGTCATAAT |
| 23 | AGGGGTGGACACCTGCTCAACAGAGAATTCGACCCCATCCTCTGGCCCTCAGTTCTGTCC |
| 24 | AGCCTCTTCCAACACTGAGGATCACAACTCGATGATATAATACTCTGCTGACTACATTTT |
| 25 | GGAGCTAAGATGACTAATTTGATGATTTTCGAACTCCTGACCTCGTGATCTTCCCACCTT |
| 26 | TCAAATGAGCATCCCCATTCTGTTTACATCGAGACCAGCCTGGCCAAAATGGTGAAAACC |
| 27 | TGCCAGCTTTTGTTGCCATTCTGGCCATTCGACAATATTGTTTTATTTTAGCATCAATTT |
| 28 | TGACTCACGAGGGAAGTGGTTGTTGACCTCGATTCCTCTCCTCTCAGTGACAACCAAAGA |
| 29 | TTTTTAAAAACCCTATACAAATGCTGATTCGACTGCATCAGCCTCTACGGGAATCTCCTC |
| 30 | ACATTCCACTTAACTGCAACTTGGCTTCTCGAATGTGGTAGCCACAGACTGCAGTCAGGA |
| 31 | AACTTGACAACCTGTAAGGGATTCTGAATCGACAGGGGAGAAAGTGCCGGCCCAGAGAAA |
| 32 | CATATACAGCCAGTCCCTGAGGAAGGAGTCGATGAAGGAAGAAAAGAGACGTGGTTTTCT |
| 33 | GCACGATATTTTTGCAAGAGTAGACAAATCGAAGACCCGAGCCGGCGCTTGCGCACTTAG |
| 34 | CCTGGCTCCTTCCTGTATTATGCTCCCTTCGAGTATTCTTCTGGGTGTTTCTATGAGGAT |
| 35 | CAAATATTAAATAACTGCTAAAAGTGCTTCGAACTCCTGAGCTCAGGTGATCCACCCACC |
| 36 | TCAGTCTCACAAGCCATATTTCAAGTGCTCGAGAACCAAATAGCCTAAGTCAATAAAATA |
| 37 | AACAGTTGTCATCTATGTAATGGACACATCGAACTCCTGACCTTGTGATCAGCCCGTCTC |
| 38 | CACCAGGCCCAGGTTCTCGCAGAAAGGCTCGAACTTGCTAGACAAAGACTTTGAATCAGT |
| 39 | TTCATAGTGCCTGGAATGTAGTAGGATCTCGATTTGTATAATTTCAATTAAAACCATCCA |
| 40 | CTGCCTATCTTTGGCTCTAACCCACAGATCGAGAGATCAGGAAATATTTAGCTCAATCTT |
| 41 | AGCCAGAGGTCGTAAAACGATGATAGAATCGAACTCCTGGTCTCAAGTGATCCACCAGCC |
| 42 | CACGGAGGTCTTTTAAGCTTAGTTGGAATCGACCTTTCAGGCTCAAGCAATCCTCCCACC |
| 43 | CCTGGCTCCTTCCTGTATTATGCTCCCTTCGAGAAGTAGAATATCTGAATGTTCTTATAT |
| 44 | TGGGCAAAGAAAGATAAGGAAGGAGATTTCGATTTCTGGGCTTGGCCATTATCACCCTGG |
| 45 | CTGATAAAAGGTAACGTTATATTGTTTGTCGAGACCATGCTGGCTAATATGATGAAACCC |
| 46 | GGGTTTTGCCATGTTGGCCAGGCTGGTCTCGAGACCAGCCTGGCCAACATGGCGAAACCC |
| 47 | GGTGCTAGGAAGCAAGGATACTGCATTGTCGACTGCATTTCTGCCTTCGGGGGCTTGACC |
| 48 | AGGGTTCAACTTCAGAAAGAGGGGGCTCTCGAAGATCCCCGGGCAGCGCGAAAGGAGGTT |
| 49 | GGGGCCACATCCAATGTCTTCGTGATGTTCGAAGCACAAAGCTCACTTACTCCCGGAACG |
| 50 | AGTTTGCATAAGAGAAGAAATTAATTTTTCGACCTCCCAAAGTGCTAGGATTACAAGCGT |

TABLE 4.a4

| | Probe Location | | | | |
|---|---|---|---|---|---|
| | | Start1 | End1 | Start2 | End2 |
| 1 | 11 | 61245995 | 61246024 | 61388263 | 61388292 |
| 2 | 3 | 18695198 | 18695227 | 18731500 | 18731529 |
| 3 | 2 | 118113385 | 118113414 | 118185368 | 118185397 |
| 4 | 17 | 27395091 | 27395120 | 27471515 | 27471544 |
| 5 | 12 | 131862267 | 131862296 | 132123601 | 132123630 |
| 6 | 12 | 121451678 | 121451707 | 121471888 | 121471917 |
| 7 | 14 | 81120332 | 81120361 | 81270752 | 81270781 |
| 8 | 13 | 45340045 | 45340074 | 45393096 | 45393125 |
| 9 | 12 | 5919129 | 5919158 | 6154101 | 6154130 |
| 10 | 16 | 9911564 | 9911593 | 9955798 | 9955827 |
| 11 | 22 | 16907332 | 16907361 | 17087417 | 17087446 |
| 12 | 9 | 132006576 | 132006605 | 132139743 | 132139772 |
| 13 | 12 | 5919129 | 5919158 | 6067405 | 6067434 |
| 14 | 19 | 52965445 | 52965474 | 52976111 | 52976140 |
| 15 | 1 | 229300142 | 229300171 | 229506873 | 229506902 |
| 16 | 16 | 18252590 | 18252619 | 18400350 | 18400379 |
| 17 | 2 | 111370481 | 111370510 | 111533116 | 111533145 |
| 18 | 5 | 55190603 | 55190632 | 55308384 | 55308413 |
| 19 | 7 | 2136476 | 2136505 | 2377882 | 2377911 |
| 20 | 12 | 109961469 | 109961498 | 110004289 | 110004318 |
| 21 | 5 | 148804536 | 148804565 | 148906079 | 148906108 |
| 22 | 2 | 174610122 | 174610151 | 174648156 | 174648185 |
| 23 | 17 | 43833454 | 43833483 | 43906619 | 43906648 |
| 24 | 4 | 74079355 | 74079384 | 74117913 | 74117942 |
| 25 | 1 | 36159279 | 36159308 | 36304376 | 36304405 |
| 26 | 5 | 124633969 | 124633998 | 124857560 | 124857589 |
| 27 | 6 | 86970657 | 86970686 | 87154542 | 87154571 |
| 28 | 4 | 38133160 | 38133189 | 38166333 | 38166362 |
| 29 | 1 | 42818832 | 42818861 | 42952377 | 42952406 |
| 30 | 10 | 44948035 | 44948064 | 44963510 | 44963539 |
| 31 | 18 | 75155502 | 75155531 | 75217309 | 75217338 |
| 32 | 1 | 151547583 | 151547612 | 151762415 | 151762444 |
| 33 | X | 49053498 | 49053527 | 49288574 | 49288603 |
| 34 | 11 | 61245995 | 61246024 | 61472772 | 61472801 |
| 35 | 14 | 81532421 | 81532450 | 81604566 | 81604595 |
| 36 | 14 | 74593649 | 74593678 | 74782362 | 74782391 |
| 37 | 2 | 27481762 | 27481791 | 27635152 | 27635181 |
| 38 | 18 | 10291525 | 10291554 | 10507743 | 10507772 |
| 39 | 1 | 109683571 | 109683600 | 109790277 | 109790306 |
| 40 | 11 | 95045563 | 95045592 | 95231622 | 95231651 |
| 41 | 6 | 6751861 | 6751890 | 6983652 | 6983681 |
| 42 | 6 | 113632954 | 113632983 | 113732265 | 113732294 |
| 43 | 11 | 61227169 | 61227198 | 61388263 | 61388292 |
| 44 | 10 | 91056660 | 91056689 | 91168327 | 91168356 |
| 45 | 2 | 102104978 | 102105007 | 102374576 | 102374605 |
| 46 | 11 | 17097713 | 17097742 | 17188137 | 17188166 |
| 47 | 1 | 199677020 | 199677049 | 199811402 | 199811431 |
| 48 | 7 | 100090608 | 100090637 | 100119091 | 100119120 |
| 49 | 12 | 7870903 | 7870932 | 7933480 | 7933509 |
| 50 | 7 | 87737453 | 87737482 | 87872696 | 87872725 |

TABLE 4.a5

| | 4 kb Sequence Location | | | | | |
|---|---|---|---|---|---|---|
| | | Start1 | End1 | Start2 | End2 | probe |
| 1 | 11 | 61242025 | 61246024 | 61388263 | 61392262 | ORF1_11_61243450_61246026_61388261_61391790_FR |
| 2 | 3 | 18691228 | 18695227 | 18731500 | 18735499 | ORF1_3_18693806_18695229_18731498_18743005_FR |
| 3 | 2 | 118109415 | 118113414 | 118185368 | 118189367 | ORF1_2_118112112_118113416_118185366_118187110_FR |
| 4 | 17 | 27395091 | 27399090 | 27471515 | 27475514 | ORF1_17_27395089_27399400_27471513_27474666_RR |
| 5 | 12 | 131862267 | 131866266 | 132119631 | 132123630 | ORF1_12_131862265_131863764_132122269_132123632_RF |
| 6 | 12 | 121447708 | 121451707 | 121467918 | 121471917 | ORF1_12_121447890_121451709_121468997_121471919_FF |
| 7 | 14 | 81120332 | 81124331 | 81266782 | 81270781 | ORF1_14_81120330_81122262_81268937_81270783_RF |
| 8 | 13 | 45336075 | 45340074 | 45393096 | 45397095 | ORF1_13_45338339_45340076_45393094_45398989_FR |
| 9 | 12 | 5915159 | 5919158 | 6150131 | 6154130 | ORF1_12_5915876_5919160_6152139_6154132_FF |
| 10 | 16 | 9911564 | 9915563 | 9951828 | 9955827 | ORF1_16_9911562_9916434_9954809_9955829_RF |
| 11 | 22 | 16907332 | 16911331 | 17087417 | 17091416 | ORF1_22_16907330_16914217_17087415_17089015_RR |
| 12 | 9 | 132002606 | 132006605 | 132139743 | 132143742 | ORF1_9_131999122_132006607_132139741_132142158_FR |
| 13 | 12 | 5915159 | 5919158 | 6063435 | 6067434 | ORF1_12_5915876_5919160_6059110_6067436_FF |
| 14 | 19 | 52961475 | 52965474 | 52976111 | 52980110 | ORF1_19_52960383_52965476_52976109_52979684_FR |
| 15 | 1 | 229296172 | 229300171 | 229506873 | 229510872 | ORF1_1_229299065_229300173_229506871_229512794_FR |
| 16 | 16 | 18252590 | 18256589 | 18400350 | 18404349 | ORF1_16_18252588_18255195_18400348_18402976_RR |
| 17 | 2 | 111366511 | 111370510 | 111529146 | 111533145 | ORF1_2_111366352_111370512_111529947_111533147_FF |
| 18 | 5 | 55186633 | 55190632 | 55308384 | 55312383 | ORF1_5_55186470_55190634_55308382_55310405_FR |
| 19 | 7 | 2132506 | 2136505 | 2373912 | 2377911 | ORF1_7_2130888_2136507_2376124_2377913_FF |
| 20 | 12 | 109957499 | 109961498 | 110000319 | 110004318 | ORF1_12_109959287_109961500_109999941_110004320_FF |
| 21 | 5 | 148804536 | 148808535 | 148902109 | 148906108 | ORF1_5_148804534_148807399_148901266_148906110_RF |
| 22 | 2 | 174610122 | 174614121 | 174648156 | 174652155 | ORF1_2_174610120_174616390_174648154_174652062_RR |
| 23 | 17 | 43829484 | 43833483 | 43902649 | 43906648 | ORF1_17_43832124_43833485_43905109_43906650_FF |
| 24 | 4 | 74075385 | 74079384 | 74117913 | 74121912 | ORF1_4_74078231_74079386_74117911_74125141_FR |
| 25 | 1 | 36159279 | 36163278 | 36300406 | 36304405 | ORF1_1_36159277_36165949_36303183_36304407_RF |
| 26 | 5 | 124629999 | 124633998 | 124857560 | 124861559 | ORF1_5_124631740_124634000_124857558_124858678_FR |
| 27 | 6 | 86970657 | 86974656 | 87154542 | 87158541 | ORF1_6_86970655_86977787_87154540_87156275_RR |
| 28 | 4 | 38133160 | 38137159 | 38162363 | 38166362 | ORF1_4_38133158_38135017_38161792_38166364_RF |
| 29 | 1 | 42814862 | 42818861 | 42948407 | 42952406 | ORF1_1_42817616_42818863_42949613_42952408_FF |
| 30 | 10 | 44948035 | 44952034 | 44963510 | 44967509 | ORF1_10_44948033_44949598_44963508_44966433_RR |
| 31 | 18 | 75155502 | 75159501 | 75213339 | 75217338 | ORF1_18_75155500_75159840_75214354_75217340_RF |
| 32 | 1 | 151547583 | 151551582 | 151758445 | 151762444 | ORF1_1_151547581_151552501_151755108_151762446_RF |
| 33 | X | 49053498 | 49057497 | 49284604 | 49288603 | ORF1_X_49053496_49056424_49286142_49288605_RF |
| 34 | 11 | 61242025 | 61246024 | 61472772 | 61476771 | ORF1_11_61243450_61246026_61472770_61474691_FR |
| 35 | 14 | 81528451 | 81532450 | 81600596 | 81604595 | ORF1_14_81530269_81532452_81602546_81604597_FF |
| 36 | 14 | 74589679 | 74593678 | 74778392 | 74782391 | ORF1_14_74588744_74593680_74780483_74782393_FF |
| 37 | 2 | 27481762 | 27485761 | 27631182 | 27635181 | ORF1_2_27481760_27484387_27626184_27635183_RF |
| 38 | 18 | 10287555 | 10291554 | 10503773 | 10507772 | ORF1_18_10287457_10291556_10505933_10507774_FF |
| 39 | 1 | 109679601 | 109683600 | 109786307 | 109790306 | ORF1_1_109681178_109683602_109788010_109790308_FF |
| 40 | 11 | 95041593 | 95045592 | 95227652 | 95231651 | ORF1_11_95040575_95045594_95230445_95231653_FF |
| 41 | 6 | 6747891 | 6751890 | 6983652 | 6987651 | ORF1_6_6745894_6751892_6983650_6986668_FR |
| 42 | 6 | 113632954 | 113636953 | 113728295 | 113732294 | ORF1_6_113632952_113639716_113727754_113732296_RF |
| 43 | 11 | 61223199 | 61227198 | 61388263 | 61392262 | ORF1_11_61224624_61227200_61388261_61391790_FR |
| 44 | 10 | 91056660 | 91060659 | 91164357 | 91168356 | ORF1_10_91056658_91062643_91164372_91168358_RF |
| 45 | 2 | 102101008 | 102105007 | 102374576 | 102378575 | ORF1_2_102100064_102105009_102374574_102377537_FR |
| 46 | 11 | 17097713 | 17101712 | 17188137 | 17192136 | ORF1_11_17097711_17100441_17188135_17191681_RR |
| 47 | 1 | 199677020 | 199681019 | 199811402 | 199815401 | ORF1_1_199677018_199683841_199811400_199820398_RR |
| 48 | 7 | 100090608 | 100094607 | 100119091 | 100123090 | ORF1_7_100090606_100091968_100119089_100121546_RR |
| 49 | 12 | 7866933 | 7870932 | 7929510 | 7933509 | ORF1_12_7868599_7870934_7932397_7933511_FF |
| 50 | 7 | 87733483 | 87737482 | 87868726 | 87872725 | ORF1_7_87727621_87737484_87868535_87872727_FF |

TABLE 4.a6

| | Primer ID | Sequence | Primer ID | Sequence |
|---|---|---|---|---|
| 1 | OBD168-1361 | TGCATGAGATGAACCAGGGTGG | OBD168-1363 | AGGTATTTCTTCTTTTTCTATT |
| 2 | OBD168-913 | CTCCTTGTTCAGACTCAGGGAATAAG | OBD168-915 | GGCTCCAGGTCTCTTCTGTCTCTTGA |
| 3 | OBD168-1925 | GTGGGAAGGAAGGGTGTCATCTATAC | OBD168-1927 | CATAAAGCCTCTCTCCAATCGCTTTC |
| 4 | OBD168-1093 | AGCAGATTAGAACCACAGTGAGTTGC | OBD168-1095 | GGAGGACACCTGTTTGCTGAGTTGGT |
| 5 | OBD168-1389 | ACAGATACAAATAAGCTTGACC | OBD168-1391 | TGTACAGTCCCAGCTACTCAGG |
| 6 | OBD168-1385 | ACTGCAGCACTATTCACAACA | OBD168-1387 | ATCCTTTTAATGTTGAGACCA |
| 7 | OBD168-581 | GATGCTTCCCAAGGCTACCTGGT | OBD168-583 | CAGAAAATCAGAAAGCCTCCACCAAA |
| 8 | OBD168-513 | CTGCGAAAGAACCTCAAAAGTTAGCC | OBD168-515 | TGGGATAATCTTACTCATTGCCTGGC |
| 9 | OBD168-533 | TTGTTATTCCAGGTGGCAATGCTGAG | OBD168-535 | GGCAGGACTTACCCAACATCAGGACT |
| 10 | OBD168-485 | GCTTGTTTTGAATGAGGTTAGGCAGC | OBD168-487 | GCCATCACAGGTTAGGAGACAGACC |
| 11 | OBD168-1585 | CACCCCTTCCCCTTGCATTGGTTTCC | OBD168-1587 | GCTGACCGTTCTTTCCTTCATTCATG |
| 12 | OBD168-813 | CCAGGAACAAAACAAACCCCAGGTAA | OBD168-815 | AAGAAGCCAGGCAAGAAGACACGAGC |
| 13 | OBD168-1793 | ACACTTATTAAGCACCTATATGTG | OBD168-1795 | TTCAGAAATTATCATCATTCTACA |
| 14 | OBD168-1509 | CCAGCCTGGGCAACAGAGTGAGACTG | OBD168-1511 | CTGTCACTCGGGCTACAGTGCAGTGG |
| 15 | OBD168-925 | GAGCCAGCCAGTCCTCATCAAGG | OBD168-927 | GCCCTTAGCCCTTCTGTATGTGC |
| 16 | OBD168-1185 | GGTGCCACAGTGCCTTACATCAC | OBD168-1187 | TTTCTGGGTCACTCCCGCCTGTG |
| 17 | OBD168-1561 | GCCTGGTGAACTCTGTCGTGTGGG | OBD168-1563 | GTGATTCATAAAGCAGCTCTTTTC |
| 18 | OBD168-1173 | CGGGCTGTCTCCTGAATGGTGTG | OBD168-1175 | TGTGCCAGGTGTTGTGCTGATGC |
| 19 | OBD168-2057 | GCAGGATCAGTGCCAGGGCAGGGGG | OBD168-2059 | GGAGGAAGGAGCAGGCGCGAGCGCT |
| 20 | OBD168-1805 | CCAGGGCGCTGTGGTTTTGCGTGGC | OBD168-1807 | AAGAAAAGTGGATGAGCTGAAATGA |
| 21 | OBD168-673 | GAGGGCTTCTGTGAGATTGGGCAC | OBD168-675 | GGCAGAGAGGCAATGTCAGGAAG |
| 22 | OBD168-661 | GAAAAGAGTCAACACGGAGCAGAAAG | OBD168-663 | GCACCACCCAGAGCACCAACCTTATT |
| 23 | OBD168-1445 | ACCGCCGCACCCTCCGGGCCG | OBD168-1447 | TCCCACCCATCCCTCCATCCA |
| 24 | OBD168-621 | AAGTGGGAGAGTGAAGGGAGAAGGGA | OBD168-623 | CAAGCACGGTTGTAGGAGTTGTAAGT |
| 25 | OBD168-1049 | GGCGGGAGAGCGATGCTTGGATT | OBD168-1051 | CTCTCCGCTGCCTTCTTGTCCTA |
| 26 | OBD168-2025 | TAAAAGAACTACTAATTATTATGTT | OBD168-2027 | CATCCAGGCTGGAGTACACTGGCGC |
| 27 | OBD168-2037 | TTGAGCTTCACAAAGGTTCCACTGAA | OBD168-2039 | TTTCACCGTGATTTAGACTCTACTAC |
| 28 | OBD168-1013 | GGTAAGCGTAGGAGCAGAGCAAC | OBD168-1015 | GGGTGGGAAAGGTCCTCTGGTCA |
| 29 | OBD168-1313 | GGGCTCAAGTGATCCTCCTGCC | OBD168-1315 | GACCATGCAGTGAAAATGAGGA |
| 30 | OBD168-985 | CCTTGTCATAATGGGAAACTCAGGGC | OBD168-987 | CCTGGGAAGCAAAAGAAAGGAGGACA |
| 31 | OBD168-481 | GAGTTTTGGCTGCTGAAGGGAAGGCA | OBD168-483 | ATGGTCATTCACTCAAATCCTGGGC |
| 32 | OBD168-901 | CCCCAAGTCTCTCTGTCCTTCCT | OBD168-903 | CATCAAAGGGAGTGGCTCTGCCC |
| 33 | OBD168-1721 | GATCATGACACATTAATTCTTAAATT | OBD168-1723 | GTAGGAGGGGAGCGCCGGCCCGGCCG |
| 34 | OBD168-797 | CCCAGGTTGGAGGCATCTCTGAC | OBD168-799 | CCAGGCGAATCTGAAGGCAGGAT |
| 35 | OBD168-897 | GCTGTTCCAGACCTAAAACCCAATGT | OBD168-899 | AAGATGACTTCCTTCCTTAGCCCAGC |
| 36 | OBD168-645 | GAGTCTCACACTGTAGCCACGAACTG | OBD168-647 | GCTCTGCCATACAGTTCATTCTCATC |
| 37 | OBD168-1901 | GCTCTGCACACCCTGCCTAAAGTGG | OBD168-1903 | GCTCTTTGTGGGCCCAGACGGTGTT |
| 38 | OBD168-1477 | CATAAATTCCTAGTTTCCTGTGCAA | OBD168-1479 | TTTCTAATTCTACTTGGTTTCTTTT |
| 39 | OBD168-857 | CTCTCCCCTGAAGTTCCCACCTG | OBD168-859 | GCAGAAGTTGGAGCCTCTACCTC |
| 40 | OBD168-1781 | GATAAAGCTTCACCATCTCTGC | OBD168-1783 | AGGGAACAGTTTGCTCTCCTCT |
| 41 | OBD168-1645 | GAGACACGCAAGTCTTGCATCTG | OBD168-1647 | ACTCTTGTGCTCAAGTGATTAGC |
| 42 | OBD168-1669 | CTGATCTAACCCCCAAAATCCT | OBD168-1671 | AGTGCTAGCTATTTGGTCTTGA |
| 43 | OBD168-1777 | AGCTCAGTGAACATGACCCGATGGTG | OBD168-1779 | ATAGTGTAAGCTAGGTAATATTTCTT |
| 44 | OBD168-949 | GAGGAGGTAGACAGGATGCTGAG | OBD168-951 | AGCCCAGGACAAGTGTGACCAGG |
| 45 | OBD168-1269 | ACCCCGACATTCTCCACCTCCTG | OBD168-1271 | ATTCTCCTTCCTCAGCCTCCCGA |
| 46 | OBD168-1357 | TCCAGCACCCAGTGTGGAGTGCA | OBD168-1359 | CTTTTTCTTTTTTTTTGAGACAG |
| 47 | OBD168-909 | GTGTGAGCCATCTGTTTATGCCTTTT | OBD168-911 | GGGTTTGACCACAGACCTGAGAGATG |
| 48 | OBD168-2069 | AGTGTCACACAGTAAAAGGTATAG | OBD168-2071 | GGAGGAGACCCTTTGAAGGTCCCT |
| 49 | OBD168-1177 | CACCAAGAAGCGACCACAGCACA | OBD168-1179 | GAAGAACAGCACCGCCCAGCGTT |
| 50 | OBD168-2065 | GTTCCAATTTTTCAATGAGGTAA | OBD168-2067 | GGCTTGAGCAAGCCTTTCAATCC |

TABLE 4.a7

| | qPCR Primer ID | Sequence | qPCR Primer ID |
|---|---|---|---|
| 1 | OBD168-q1361 | CCTGCATGAGATGAACCAGGG | OBD168-q1363 |
| 2 | OBD168-q913 | GACTCCTTGTTCAGACTCAGGGAATAAG | OBD168-q915 |
| 3 | OBD168-q1925 | TTTTGACCATTTGAGCAGGTAC | OBD168-q1927 |
| 4 | OBD168-q1093 | AAAGCAGATTAGAACCACAGTGAGTTGC | OBD168-q1095 |
| 5 | OBD168-q1389 | TTGACAAGTCTTTTAAGAAAAAGTAC | OBD168-q1391 |
| 6 | OBD168-q1385 | CAAAACATGATCTCAAAGAGATAT | OBD168-q1387 |
| 7 | OBD168-q581 | GCCAGATGCTTCCCAAGGCTACCTG | OBD168-q583 |
| 8 | OBD168-q513 | CACTGCGAAAGAACCTCAAAAGTTAGCC | OBD168-q515 |
| 9 | OBD168-q533 | GATGAAAATGGCACCTCAAGCCTCAGCA | OBD168-q535 |
| 10 | OBD168-q485 | TGGCTTGTTTTGAATGAGGTTAGGCAGC | OBD168-q487 |
| 11 | OBD168-q1585 | TGCTGCTGTAACAAATTACCAC | OBD168-q1587 |
| 12 | OBD168-q813 | GGAAAAGGAGACTGAGGAAACGCTCTGC | OBD168-q815 |
| 13 | OBD168-q1793 | GCGAGCCAAACAAGATCTCTGCC | OBD168-q1795 |
| 14 | OBD168-q1509 | AAATTATAAACTTTTACATCACCGCA | OBD168-q1511 |
| 15 | OBD168-q925 | CCTGCCCCAGCCAGAGGATAGAGGAGG | OBD168-q927 |
| 16 | OBD168-q1185 | GGGTGCCACAGTGCCTTACATCACATCC | OBD168-q1187 |
| 17 | OBD168-q1561 | CAATGGGAAGGTGGACAGAGGA | OBD168-q1563 |
| 18 | OBD168-q1173 | GGACGGGCTGTCTCCTGAATGGTGTG | OBD168-q1175 |
| 19 | OBD168-q2057 | GCAGGCAGGATCAGTGCCAGGGCAG | OBD168-q2059 |
| 20 | OBD168-q1805 | GCTGTGGTTTTGCGTGGCAAGCCAGA | OBD168-q1807 |
| 21 | OBD168-q673 | GTCAGGTCAGCACATCCCATTAGGTGGA | OBD168-q675 |
| 22 | OBD168-q661 | GGGAAAAGAGTCAACACGGAGCAGAAAG | OBD168-q663 |
| 23 | OBD168-q1445 | CTGCCTGTGTCCCCCCGCACCCT | OBD168-q1447 |
| 24 | OBD168-q621 | GGAGAGTGAAGGGAGAAGGGACACACCC | OBD168-q623 |
| 25 | OBD168-q1049 | GGGTAGGGCGGGAGAGCGATGCTTGGAT | OBD168-q1051 |
| 26 | OBD168-q2025 | TAATTATTATGTTAAAATCTGGG | OBD168-q2027 |
| 27 | OBD168-q2037 | ACCTTTCAGAACACCTTTGTGTGC | OBD168-q2039 |
| 28 | OBD168-q1013 | GGTGAAAGGGTAAGCGTAGGAGCAGAGC | OBD168-q1015 |
| 29 | OBD168-q1313 | GGAAATCCTGGGCTCAAGTGATC | OBD168-q1315 |
| 30 | OBD168-q985 | GCTGGGCATCCCTGAGGCTTGGAGC | OBD168-q987 |
| 31 | OBD168-q481 | CAAGAGTTTTGGCTGCTGAAGGGAAGGC | OBD168-q483 |
| 32 | OBD168-q901 | CTCACCGCCTCACCTGCCTTGGTCTGG | OBD168-q903 |
| 33 | OBD168-q1721 | GTTTTGCCTGTGTGTAGATCATGAC | OBD168-q1723 |
| 34 | OBD168-q797 | CCCACCCAGGTTGGAGGCATCTCTGAC | OBD168-q799 |
| 35 | OBD168-q897 | GGATACACACCGTGCTCTTTCATTACTG | OBD168-q899 |
| 36 | OBD168-q645 | GGCAGGAGGATTGGTTTGGTTTGACCCC | OBD168-q647 |
| 37 | OBD168-q1901 | AAGAGTTTGAGGACAGATCGTGAG | OBD168-q1903 |
| 38 | OBD168-q1477 | CCTGTGCAATCTCAGTATTTT | OBD168-q1479 |
| 39 | OBD168-q857 | ACCCCTCTCCCACTCCACTGCCTCTCAG | OBD168-q859 |
| 40 | OBD168-q1781 | ATCCCATGACTTCCCGGTAGTG | OBD168-q1783 |
| 41 | OBD168-q1645 | CTTTCCCCTCCCCCTCCCCCTCCCC | OBD168-q1647 |
| 42 | OBD168-q1669 | ACTTGTATGAATGATATGTTACTG | OBD168-q1671 |
| 43 | OBD168-q1777 | AGGGTGGCTCTGGGCCTGAGGCT | OBD168-q1779 |
| 44 | OBD168-q949 | GGGAGATAGGAGGAGGTAGACAGGATGC | OBD168-q951 |
| 45 | OBD168-q1269 | GATTACCCCGACATTCTCCACCTCCTGG | OBD168-q1271 |
| 46 | OBD168-q1357 | CCTCTTTCCTCCTGGGTTCAAGC | OBD168-q1359 |
| 47 | OBD168-q909 | TAGTGTGAGCCATCTGTTTATGCCTTTT | OBD168-q911 |
| 48 | OBD168-q2069 | AAGTGGCATCTGGCATTCCCCAAT | OBD168-q2071 |
| 49 | OBD168-q1177 | CTCTGCTACCTTCCCCACCCTGCG | OBD168-q1179 |
| 50 | OBD168-q2065 | ATTTTTCAATGAGGTAAAATGAAA | OBD168-q2067 |

TABLE 4.a8

| | Sequence | qPCRProbe | Sequence |
|---|---|---|---|
| 1 | CAATAAAGCGAAAGGTATTTC | OBD168-p1361 | TCCTGTATTATGCTCCCTTCGAGAAGTAGAATATCTGAAT |
| 2 | CCTGCCAAGCCTGCCGCCACTGT | OBD168-p913 | TGGACTGGGCTAATGTGGAAAGTGAGGATCGAGAATG |
| 3 | TAAAGCCTCTCTCCAATCGCTT | OBD168-p1925 | TATTTTTATTTATTTTTTTCGATTGTGTTCACCAGGACGG |
| 4 | GTAGGAGGACACCTGTTTGCTGAGTTGG | OBD168-p1093 | ACAATTTCGAGCTTGGTGTGGTCAAGGGCCAGG |
| 5 | CAGGTGCAGTGGCACAGGCCTGTACA | OBD168-p1389 | TTTGGCTAATCAGAGATTTCGACAGGAGGATGGTGGGGAG |
| 6 | TTTTCCAGAGGCTCAGGGCAGAGA | OBD168-p1385 | TCACTTGAGGTCAGGAGTTCGAACTCCTGACCTCAAGTGA |
| 7 | CTGTTTCTGAGTGCGGATGACATTATCT | OBD168-p581 | AGGCAGAAGTTGTGGGCATGCTGAACTCGAGGAA |
| 8 | GCTGGGATAATCTTACTCATTGCCTGGC | OBD168-p513 | TGCAGGTGATGGTTCATGACAATATCGACTGAGCTGC |
| 9 | GGCAGGACTTACCCAACATCAGGACTTC | OBD168-p533 | AGGCAGGATCGATGAATTCTAGGGCATGTAAATGCTC |
| 10 | GCCATCACAGGTTAGGAGACAGACC | OBD168-p485 | ATGAGCTCCCTGTGTCACACAGCATCGAGAGTC |
| 11 | TTTGTTCAGTCAACACATATGT | OBD168-p1585 | AAGAGAGAGAATCCAATGTCGATTTAGCCTTCCATAACTA |
| 12 | GCAAGAAGCCAGGCAAGAAGACACGAGC | OBD168-p813 | TTTTGTAATCGAAGGACCCCTTGCTGGG |
| 13 | GGAATTCTTCCCCCTGCCTTTCT | OBD168-p1793 | AAGAAAAGTAAGGCAGGATCGAATATTTGCACATTTCTCG |
| 14 | TCTGTCACTCGGGCTACAGTGCAGTG | OBD168-p1509 | CACTCACATCCCTAGTTATCGAGAACATCCTGGCTAATGT |
| 15 | CTCTGTCAGGAAACAAAGCCCTTAGCCC | OBD168-p925 | TGGGCTGAAGTATGGTCGAACAGGTTACGAGCATC |
| 16 | TATTTCCCAGGCTCCAGGCTCCTCGCCG | OBD168-p1185 | ACAATGGGTCACGTGCCTATTCGAGACCACCC |
| 17 | TGTGATTCATAAAGCAGCTCTT | OBD168-p1561 | TTTTGAATCCCTCCCCCATCGAGGTTAATCCCTCTCCACA |
| 18 | GACTATGTGCCAGGTGTTGTGCTGATGC | OBD168-p1173 | ACGAGGAGGGTTTCGATGTTACTGATTCTGTCCCC |
| 19 | AATGACCCTGGGTGTCATGGAGGAA | OBD168-p2057 | CCAAGTTTCCTAACAGGATCGACACCCAGGGTCATTCACA |
| 20 | CAAGAAAAGTGGATGAGCTGAAATGA | OBD168-p1805 | CACACGCAAAGGCAAACGTCGAAACTGATCATTTAGGTTT |
| 21 | AAAGGCAGAGAGGCAATGTCAGGAAGAC | OBD168-p675 | TTCCCCTCGAGAAGAGCCTGCATCCAAGGTCT |
| 22 | CTTTAGGCACCACCCAGAGCACCAACC | OBD168-p661 | TCCTGTGGCCTGATAAGGTATTGACCCCATCGAGATT |
| 23 | CATCACCCATAGTGTCCGCAGGC | OBD168-p1445 | ACCTGCTCAACAGAGAATTCGACCCCATCCTCTGGCCCTC |
| 24 | GTGTGAATCAGGAGAGGACAAAAGAGGG | OBD168-p621 | AGCCTCTTCCAACACTGAGGATCACAACTCGATGAT |
| 25 | CCCCTCTCCGCTGCCTTCTTGTCC | OBD168-p1049 | TGATTTTCGAACTCCTGACCTCGTGATCTTCCCACC |
| 26 | CTCCCGGGTTCAAATGATCCTTC | OBD168-p2025 | ATCCCCATTCTGTTTACATCGAGACCAGCCTGGCCAAAAT |
| 27 | GTTTCACCGTGATTTAGACTCTAC | OBD168-p2037 | TCTTGCCATTCTGGCCATTCGACAATATTGTTTTATTTTA |
| 28 | GCTCTGGGACTGCCTGACCTTAGCAAAG | OBD168-p1013 | ACTCACGAGGGAAGTGGTTGTTGACCTCGATTCC |
| 29 | TTCTGTGGCTGGTGGGCTCAAAG | OBD168-p1313 | CCCTATACAAATGCTGATTCGACTGCATCAGCCTCTACGG |
| 30 | GCCACGCTGACGGCATTCCTGGG | OBD168-p985 | TGGCTTCTCGAATGTGGTAGCCACAGACTGCAG |
| 31 | CCCCAACCCCAGCACAACCAGCCCTG | OBD168-p481 | ACTTGACAACCTGTAAGGGATTCTGAATCGACAGGG |
| 32 | CACCCACTTGCTAAGCCAGTCTCACTCT | OBD168-p901 | ACAGCCAGTCCCTGAGGAAGGAGTCGATGAAG |
| 33 | GTCTGTCCGCTTCCAGCGGCACGGC | OBD168-p1721 | TTTGCAAGAGTAGACAAATCGAAGACCCGAGCCGGCGCTT |
| 34 | GCCAGTGTTCCCAGGCGAATCTGAAGGC | OBD168-p797 | ACCTGGCTCCTTCCTGTATTATGCTCCCTTCGAGTAT |
| 35 | CCAAGATGACTTCCTTCCTTAGCCCAGC | OBD168-p897 | AAATAACTGCTAAAAGTGCTTCGAACTCCTGAGCTC |
| 36 | GCCCCACCGACAACTTACCATCCTCCG | OBD168-p645 | TCAGTCTCACAAGCCATATTTCAAGTGCTCGAGAACC |
| 37 | TGACCCTCCTTAAAAAAAGAAGCC | OBD168-p1901 | ATCTATGTAATGGACACATCGAACTCCTGACCTTGTGATC |
| 38 | CAGCTCCAGGATTTCTAATTC | OBD168-p1477 | AGGTTCTCGCAGAAAGGCTCGAACTTGCTAGACAAAGACT |
| 39 | GTGGGAACGGGTCAGCGTCCTGG | OBD168-p857 | TTTATTCATAGTGCCTGGAATGTAGTAGGATCTCGATTTG |
| 40 | CCAATAAGAGGACGAGGGTGTG | OBD168-p1781 | TTGGCTCTAACCCACAGATCGAGAGATCAGGAAATATTTA |
| 41 | CCCAGCCTGATCTCAGACTCTTGTG | OBD168-p1645 | CGTAAAACGATGATAGAATCGAACTCCTGGTCTCAAGTGA |
| 42 | GGCTTCAAGCAATCCTCCCACCTC | OBD168-p1669 | TTTTAAGCTTAGTTGGAATCGACCTTTCAGGCTCAAGCAA |
| 43 | ATTCTCCGGAAGAGATAGTGTAA | OBD168-p1777 | TCCTGTATTATGCTCCCTTCGAGAAGTAGAATATCTGAAT |
| 44 | GCCCAGCAGTCAAGGTTGGAAGCCCAGG | OBD168-p949 | AGGAGATTTCGATTTCTGGGCTTGGCCATTATCACCC |
| 45 | TGATTCTCCTTCCTCAGCCTCCCGA | OBD168-p1269 | TGTTTGTCGAGACCATGCTGGCTAATATGATGAAACCC |
| 46 | CACCCAGGTTGGAGTGTAGTGGT | OBD168-p1357 | ATGTTGGCCAGGCTGGTCTCGAGACCAGCCTGGCCAACAT |
| 47 | AGGGTTTGACCACAGACCTGAGAGATGA | OBD168-p909 | AGGATACTGCATTGTCGACTGCATTTCTGCCTTCGG |
| 48 | CCCCGGAGGAGACCCTTTGAAGGT | OBD168-p2069 | TTCAGAAAGAGGGGGCTCTCGAAGATCCCCGGGCAGCGCG |
| 49 | GGGAGGAAGAACAGCACCGCCCAGCGTT | OBD168-p1177 | TCGTGATGTTCGAAGCACAAAGCTCACTTACTCCCG |
| 50 | ATCCTTTTCAATGAAAGGGAAATT | OBD168-p2065 | AGAGAAGAAATTAATTTTTCGACCTCCCAAAGTGCTAGGA |

TABLE 4.a9

| | qPCR Probe | Sequence |
|---|---|---|
| 1 | OBD168-p1363 | ATTCAGATATTCTACTTCTCGAAGGGAGCATAATACAGGA |
| 2 | OBD168-p915 | TCATTCTCGATCCTCACTTTCCACATTAGCCCAGTCC |
| 3 | OBD168-p1927 | CCGTCCTGGTGAACACAATCGAAAAAAATAAATAAAAATA |
| 4 | OBD168-p1095 | TGGCCCTTGACCACACCAAGCTCGAAATTGTCAG |
| 5 | OBD168-p1391 | CTCCCCACCATCCTCCTGTCGAAATCTCTGATTAGCCAAA |
| 6 | OBD168-p1387 | TCACTTGAGGTCAGGAGTTCGAACTCCTGACCTCAAGTGA |
| 7 | OBD168-p583 | TTCCTCGAGTTCAGCATGCCCACAACTTCTGCC |
| 8 | OBD168-p515 | TGGCAGCTCAGTCGATATTGTCATGAACCATCACCTG |
| 9 | OBD168-p535 | TGAGCATTTACATGCCCTAGAATTCATCGATCCTGCC |
| 10 | OBD168-p487 | AGGACTCTCGATGCTGTGTGACACAGGGAGCT |
| 11 | OBD168-p1587 | TAGTTATGGAAGGCTAAATCGACATTGGATTCTCTCTCTT |
| 12 | OBD168-p815 | CCCAGCAAGGGGTCCTTCGATTACAAAA |
| 13 | OBD168-p1795 | CGAGAAATGTGCAAATATTCGATCCTGCCTTACTTTTCTT |
| 14 | OBD168-p1511 | ACATTAGCCAGGATGTTCTCGATAACTAGGGATGTGAGTG |
| 15 | OBD168-p927 | TGATGCTCGTAACCTGTTCGACCATACTTCAGCCC |
| 16 | OBD168-p1187 | TGGGTGGTCTCGAATAGGCACGTGACCCATTG |
| 17 | OBD168-p1563 | TGTGGAGAGGGATTAACCTCGATGGGGGAGGGATTCAAAA |
| 18 | OBD168-p1175 | AACATCGAAACCCTCCTCGTATTTGTTACTCTCTCTAC |
| 19 | OBD168-p2059 | TGTGAATGACCCTGGGTGTCGATCCTGTTAGGAAACTTGG |
| 20 | OBD168-p1807 | AAACCTAAATGATCAGTTTCGACGTTTGCCTTTGCGTGTG |
| 21 | OBD168-p673 | AGACCTTGGATGCAGGCTCTTCTCGAGGGGAA |
| 22 | OBD168-p663 | AATCTCGATGGGGTCAATACCTTATCAGGCCACAGGA |
| 23 | OBD168-p1447 | GAGGGCCAGAGGATGGGGTCGAATTCTCTGTTGAGCAGGT |
| 24 | OBD168-p623 | ATCATCGAGTTGTGATCCTCAGTGTTGGAAGAGGCTC |
| 25 | OBD168-p1051 | AGGTGGGAAGATCACGAGGTCAGGAGTTCGAAAATCA |
| 26 | OBD168-p2027 | ATTTTGGCCAGGCTGGTCTCGATGTAAACAGAATGGGGAT |
| 27 | OBD168-p2039 | TAAAATAAAACAATATTGTCGAATGGCCAGAATGGCAAGA |
| 28 | OBD168-p1015 | AGGAATCGAGGTCAACAACCACTTCCCTCGTGAG |
| 29 | OBD168-p1315 | CCGTAGAGGCTGATGCAGTCGAATCAGCATTTGTATAGGG |
| 30 | OBD168-p987 | TGCAGTCTGTGGCTACCACATTCGAGAAGCCAAG |
| 31 | OBD168-p483 | TCTCCCCTGTCGATTCAGAATCCCTTACAGGTTGTC |
| 32 | OBD168-p903 | TCCTTCATCGACTCCTTCCTCAGGGACTGGCT |
| 33 | OBD168-p1723 | AAGCGCCGGCTCGGGTCTTCGATTTGTCTACTCTTGCAAA |
| 34 | OBD168-p799 | ATACTCGAAGGGAGCATAATACAGGAAGGAGCCAGG |
| 35 | OBD168-p899 | AGCTCAGGAGTTCGAAGCACTTTTAGCAGTTATTTA |
| 36 | OBD168-p647 | TGGTTCTCGAGCACTTGAAATATGGCTTGTGAGACTG |
| 37 | OBD168-p1903 | GATCACAAGGTCAGGAGTTCGATGTGTCCATTACATAGAT |
| 38 | OBD168-p1479 | AGTCTTTGTCTAGCAAGTTCGAGCCTTTCTGCGAGAACCT |
| 39 | OBD168-p859 | ACAAATCGAGATCCTACTACATTCCAGGCACTATGAATAA |
| 40 | OBD168-p1783 | TAAATATTTCCTGATCTCTCGATCTGTGGGTTAGAGCCAA |
| 41 | OBD168-p1647 | TCACTTGAGACCAGGAGTTCGATTCTATCATCGTTTTACG |
| 42 | OBD168-p1671 | TTGCTTGAGCCTGAAAGGTCGATTCCAACTAAGCTTAAAA |
| 43 | OBD168-p1779 | ATTCAGATATTCTACTTCTCGAAGGGAGCATAATACAGGA |
| 44 | OBD168-p951 | AGGGTGATAATGGCCAAGCCCAGAAATCGAAATCTCC |
| 45 | OBD168-p1271 | TCATATTAGCCAGCATGGTCTCGACAAACAATATAACGT |
| 46 | OBD168-p1359 | ATGTTGGCCAGGCTGGTCTCGAGACCAGCCTGGCCAACAT |
| 47 | OBD168-p911 | TGCAGTCGACAATGCAGTATCCTTGCTTCCTAGCAC |
| 48 | OBD168-p2071 | CGCGCTGCCCGGGGATCTTCGAGAGCCCCCTCTTTCTGAA |
| 49 | OBD168-p1179 | TGCTTCGAACATCACGAAGACATTGGATGTGGCCC |
| 50 | OBD168-p2067 | TCCTAGCACTTTGGGAGGTCGAAAAATTAATTTCTTCTCT |

TABLE 4.a10

| | probe | PCR Marker Set | qPCR Marker Set |
|---|---|---|---|
| 1 | ORF1_11_61243450_61246026_61388261_61391790_FR | OBD168-1361.1363 | OBD168-q1361.q1363.p1361/p1363 |
| 2 | ORF1_3_18693806_18695229_18731498_18743005_FR | OBD168-913.915 | OBD168-q913.q915.p913/p915 |
| 3 | ORF1_2_118112112_118113416_118185366_118187110_FR | OBD168-1925.1927 | OBD168-q1925.q1927.p1925/p1927 |
| 4 | ORF1_17_27395089_27399400_27471513_27474666_RR | OBD168-1093.1095 | OBD168-q1093.q1095.p1093/p1095 |
| 5 | ORF1_12_131862265_131863764_132122269_132123632_RF | OBD168-1389.1391 | OBD168-q1389.q1391.p1389/p1391 |
| 6 | ORF1_12_121447890_121451709_121468997_121471919_FF | OBD168-1385.1387 | OBD168-q1385.q1387.p1385/p1387 |
| 7 | ORF1_14_81120330_81122262_81268937_81270783_RF | OBD168-581.583 | OBD168-q581.q583.p581/p583 |
| 8 | ORF1_13_45338339_45340076_45393094_45398989_FR | OBD168-513.515 | OBD168-q513.q515.p513/p515 |
| 9 | ORF1_12_5915876_5919160_6152139_6154132_FF | OBD168-533.535 | OBD168-q533.q535.p533/p535 |
| 10 | ORF1_16_9911562_9916434_9954809_9955829_RF | OBD168-485.487 | OBD168-q485.q487.p485/p487 |
| 11 | ORF1_22_16907330_16914217_17087415_17089015_RR | OBD168-1585.1587 | OBD168-q1585.q1587.p1585/p1587 |
| 12 | ORF1_9_131999122_132006607_132139741_132142158_FR | OBD168-813.815 | OBD168-q813.q815.p813/p815 |
| 13 | ORF1_12_5915876_5919160_6059110_6067436_FF | OBD168-1793.1795 | OBD168-q1793.q1795.p1793/p1795 |
| 14 | ORF1_19_52960383_52965476_52976109_52979684_FR | OBD168-1509.1511 | OBD168-q1509.q1511.p1509/p1511 |
| 15 | ORF1_1_229299065_229300173_229506871_229512794_FR | OBD168-925.927 | OBD168-q925.q927.p925/p927 |
| 16 | ORF1_16_18252588_18255195_18400348_18402976_RR | OBD168-1185.1187 | OBD168-q1185.q1187.p1185/p1187 |
| 17 | ORF1_2_111366352_111370512_111529947_111533147_FF | OBD168-1561.1563 | OBD168-q1561.q1563.p1561/p1563 |
| 18 | ORF1_5_55186470_55190634_55308382_55310405_FR | OBD168-1173.1175 | OBD168-q1173.q1175.p1173/p1175 |
| 19 | ORF1_7_2130888_2136507_2376124_2377913_FF | OBD168-2057.2059 | OBD168-q2057.q2059.p2057/p2059 |
| 20 | ORF1_12_109959287_109961500_109999941_110004320_FF | OBD168-1805.1807 | OBD168-q1805.q1807.p1805/p1807 |
| 21 | ORF1_5_148804534_148807399_148901266_148906110_RF | OBD168-673.675 | OBD168-q673.q675.p675/p673 |
| 22 | ORF1_2_174610120_174616390_174648154_174652062_RR | OBD168-661.663 | OBD168-q661.q663.p661/p663 |
| 23 | ORF1_17_43832124_43833485_43905109_43906650_FF | OBD168-1445.1447 | OBD168-q1445.q1447.p1445/p1447 |
| 24 | ORF1_4_74078231_74079386_74117911_74125141_FR | OBD168-621.623 | OBD168-q621.q623.p621/p623 |
| 25 | ORF1_1_36159277_36165949_36303183_36304407_RF | OBD168-1049.1051 | OBD168-q1049.q1051.p1049/p1051 |
| 26 | ORF1_5_124631740_124634000_124857558_124858678_FR | OBD168-2025.2027 | OBD168-q2025.q2027.p2025/p2027 |
| 27 | ORF1_6_86970655_86977787_87154540_87156275_RR | OBD168-2037.2039 | OBD168-q2037.q2039.p2037/p2039 |
| 28 | ORF1_4_38133158_38135017_38161792_38166364_RF | OBD168-1013.1015 | OBD168-q1013.q1015.p1013/p1015 |
| 29 | ORF1_1_42817616_42818863_42949613_42952408_FF | OBD168-1313.1315 | OBD168-q1313.q1315.p1313/p1315 |
| 30 | ORF1_10_44948033_44949598_44963508_44966433_RR | OBD168-985.987 | OBD168-q985.q987.p985/p987 |
| 31 | ORF1_18_75155500_75159840_75214354_75217340_RF | OBD168-481.483 | OBD168-q481.q483.p481/p483 |
| 32 | ORF1_1_151547581_151552501_151755108_151762446_RF | OBD168-901.903 | OBD168-q901.q903.p901/p903 |
| 33 | ORF1_X_49053496_49056424_49286142_49288605_RF | OBD168-1721.1723 | OBD168-q1721.q1723.p1721/p1723 |
| 34 | ORF1_11_61243450_61246026_61472770_61474691_FR | OBD168-797.799 | OBD168-q797.q799.p797/p799 |
| 35 | ORF1_14_81530269_81532452_81602546_81604597_FF | OBD168-897.899 | OBD168-q897.q899.p897/p899 |
| 36 | ORF1_14_74588744_74593680_74780483_74782393_FF | OBD168-645.647 | OBD168-q645.q647.p645/p647 |
| 37 | ORF1_2_27481760_27484387_27626184_27635183_RF | OBD168-1901.1903 | OBD168-q1901.q1903.p1901/p1903 |
| 38 | ORF1_18_10287457_10291556_10505933_10507774_FF | OBD168-1477.1479 | OBD168-q1477.q1479.p1477/p1479 |
| 39 | ORF1_1_109681178_109683602_109788010_109790308_FF | OBD168-857.859 | OBD168-q857.q859.p857/p859 |
| 40 | ORF1_11_95040575_95045594_95230445_95231653_FF | OBD168-1781.1783 | OBD168-q1781.q1783.p1781/p1783 |
| 41 | ORF1_6_6745894_6751892_6983650_6986668_FR | OBD168-1645.1647 | OBD168-q1645.q1647.p1645/p1647 |
| 42 | ORF1_6_113632952_113639716_113727754_113732296_RF | OBD168-1669.1671 | OBD168-q1669.q1671.p1669/p1671 |
| 43 | ORF1_11_61224624_61227200_61388261_61391790_FR | OBD168-1777.1779 | OBD168-q1777.q1779.p1777/p1779 |
| 44 | ORF1_10_91056658_91062643_91164372_91168358_RF | OBD168-949.951 | OBD168-q949.q951.p949/p951 |
| 45 | ORF1_2_102100064_102105009_102374574_102377537_FR | OBD168-1269.1271 | OBD168-q1269.q1271.p1269/p1271 |
| 46 | ORF1_11_17097711_17100441_17188135_17191681_RR | OBD168-1357.1359 | OBD168-q1357.q1359.p1357/p1359 |
| 47 | ORF1_1_199677018_199683841_199811400_199820398_RR | OBD168-909.911 | OBD168-q909.q911.p909/p911 |
| 48 | ORF1_7_100090606_100091968_100119089_100121546_RR | OBD168-2069.2071 | OBD168-q2069.q2071.p2069/p2071 |
| 49 | ORF1_12_7868599_7870934_7932397_7933511_FF | OBD168-1177.1179 | OBD168-q1177.q1179.p1177/p1179 |
| 50 | ORF1_7_87727621_87737484_87868535_87872727_FF | OBD168-2065.2067 | OBD168-q2065.q2067.p2065/p2067 |

TABLE 4.b1

| | | | |
|---|---|---|---|
| 51 | ORF1_12_51666821_51670814_51862305_51869704_FR | 401 | 3075.288832 |
| 52 | ORF1_7_137931629_137933966_137981878_137983291_FR | 323 | 2644.935118 |
| 53 | ORF1_11_61042263_61044402_61243450_61246026_FF | 245 | 2203.942492 |
| 54 | ORF1_X_2888191_2891631_3046105_3049187_RF | 220 | 2093.173181 |
| 55 | ORF1_11_115616166_115618345_115711125_115715158_FF | 359 | 2842.705641 |
| 56 | ORF1_X_77646855_77652042_77915112_77916580_RF | 430 | 3219.053584 |
| 57 | ORF1_20_62658422_62663845_62934812_62935956_RF | 419 | 3172.19168 |
| 58 | ORF1_22_17921708_17928023_17999187_18001605_FF | 398 | 3062.496857 |
| 59 | ORF1_19_47777294_47778517_47957709_47959307_RF | 364 | 2875.54375 |
| 60 | ORF1_8_7882531_7883805_7954782_7966362_FF | 266 | 2337.981624 |
| 61 | ORF1_11_12091217_12094358_12153671_12160688_FR | 298 | 2559.990469 |
| 62 | ORF1_17_31222339_31224131_31324781_31330350_RR | 288 | 2501.124218 |
| 63 | ORF1_7_4620261_4621735_4724415_4726417_FR | 416 | 3166.931042 |
| 64 | ORF1_10_45820548_45823847_45898343_45903899_RF | 311 | 2598.185801 |
| 65 | ORF1_16_81474879_81476453_81607034_81611259_RR | 463 | 3398.382345 |
| 66 | ORF1_11_10776569_10779775_10900691_10903608_FF | 332 | 2691.77054 |
| 67 | ORF1_8_97253466_97261198_97345458_97357412_RF | 411 | 3139.677711 |
| 68 | ORF1_1_109681178_109683602_109741801_109745521_FR | 340 | 2722.247784 |
| 69 | ORF1_1_7774817_7777030_7966809_7968736_FF | 294 | 2534.406731 |
| 70 | ORF1_12_7858281_7862390_7932397_7933511_RF | 304 | 2568.815685 |
| 71 | ORF1_13_30408291_30410407_30585632_30589891_RR | 310 | 2592.51634 |
| 72 | ORF1_15_30288521_30290733_30544055_30547496_FR | 375 | 2927.700157 |
| 73 | ORF1_11_61152527_61154904_61243450_61246026_RF | 316 | 2615.722841 |
| 74 | ORF1_17_64322183_64329531_64384310_64385587_RF | 483 | 3472.308017 |
| 75 | ORF1_6_26459325_26460705_26493174_26499033_RR | 449 | 3319.91788 |
| 76 | ORF1_2_74503923_74507137_74519716_74521847_FF | 514 | 3577.121944 |
| 77 | ORF1_2_159230477_159238639_159250572_159255062_FF | 498 | 3520.298247 |
| 78 | ORF1_16_46899253_46904082_47060044_47061469_FF | 459 | 3384.670678 |
| 79 | ORF1_5_40609009_40613245_40692971_40695207_FR | 341 | 2735.33663 |
| 80 | ORF1_4_47908113_47912137_48013964_48015665_FF | 354 | 2796.807098 |
| 81 | ORF1_4_38161792_38166364_38180538_38182770_FR | 357 | 2829.764831 |
| 82 | ORF1_7_22482163_22488443_22587350_22592472_RR | 504 | 3548.464077 |
| 83 | ORF1_17_27477797_27480875_27629083_27630392_RR | 373 | 2916.46944 |
| 84 | ORF1_10_13707193_13712319_13854651_13857653_FR | 365 | 2875.544458 |
| 85 | ORF1_15_79006295_79007772_79123113_79127341_RF | 541 | 3719.882512 |
| 86 | ORF1_19_9777468_9779791_9828343_9833999_FF | 383 | 2978.506253 |
| 87 | ORF1_17_48576277_48578280_48760984_48766661_FF | 500 | 3523.786793 |
| 88 | ORF1_3_124942088_124944537_125145442_125149201_RR | 406 | 3118.518158 |
| 89 | ORF1_2_230319552_230322976_230433766_230436650_RR | 403 | 3095.482353 |
| 90 | ORF1_5_179455074_179456297_179632102_179634386_RF | 525 | 3614.075373 |
| 91 | ORF1_13_94395452_94397500_94478315_94481582_FR | 421 | 3181.350595 |
| 92 | ORF1_1_185482953_185486658_185569901_185571399_FF | 426 | 3194.213302 |
| 93 | ORF1_7_105801948_105809991_105902395_105905257_RF | 423 | 3184.944399 |
| 94 | ORF1_17_75411072_75413612_75649583_75651419_RF | 434 | 3237.927886 |
| 95 | ORF1_11_61224624_61227200_61378098_61379415_FF | 415 | 3162.182868 |
| 96 | ORF1_5_94419239_94427299_94505842_94514405_FR | 551 | 3764.075451 |
| 97 | ORF1_17_82434207_82440644_82511425_82512972_FR | 567 | 3825.12433 |
| 98 | ORF1_17_59893718_59895987_59916319_59918152_FR | 568 | 3830.891167 |
| 99 | ORF1_2_102051982_102057593_102268036_102274328_RR | 543 | 3724.636088 |
| 100 | ORF1_1_203288566_203291154_203473865_203477190_FR | 577 | 3888.735935 |

TABLE 4.b2

| | | | | |
|---|---|---|---|---|
| 51 | −1.437023753 | 1.444662839 | 0.00410465 | Late SSc |
| 52 | −1.436812218 | 1.372546527 | 0.003141195 | Late SSc |
| 53 | −1.435876536 | 1.307037927 | 0.002268922 | Late SSc |
| 54 | −1.433613438 | 1.327259391 | 0.00206892 | Late SSc |
| 55 | −1.432745201 | 1.403705162 | 0.003570554 | Late SSc |
| 56 | −1.429058783 | 1.46064713 | 0.004450195 | Late SSc |
| 57 | −1.426850922 | 1.460623768 | 0.004336283 | Late SSc |
| 58 | −1.426020051 | 1.444849909 | 0.00407447 | Late SSc |
| 59 | −1.422034199 | 1.41292781 | 0.003644069 | Late SSc |
| 60 | −1.420867576 | 1.337778105 | 0.002521338 | Late SSc |
| 61 | −1.417944096 | 1.403736871 | 0.002963925 | Late SSc |
| 62 | −1.416627122 | 1.393528599 | 0.002843634 | Late SSc |
| 63 | −1.416315236 | 1.466844799 | 0.004323573 | Late SSc |
| 64 | −1.414963784 | 1.380989039 | 0.003043098 | Late SSc |
| 65 | −1.413911362 | 1.49285337 | 0.004897376 | Late SSc |
| 66 | −1.411966593 | 1.37767433 | 0.003240783 | Late SSc |
| 67 | −1.41025199 | 1.462162948 | 0.004257973 | Late SSc |
| 68 | −1.409458393 | 1.372450033 | 0.003306288 | Late SSc |
| 69 | −1.408443165 | 1.397614445 | 0.002911387 | Late SSc |
| 70 | −1.407950766 | 1.384488035 | 0.00298214 | Late SSc |
| 71 | −1.404913131 | 1.380068244 | 0.00303129 | Late SSc |
| 72 | −1.403981089 | 1.415911341 | 0.00376212 | Late SSc |
| 73 | −1.403771448 | 1.375504908 | 0.003079743 | Late SSc |
| 74 | −1.403471491 | 1.486407014 | 0.005086864 | Late SSc |
| 75 | −1.401945125 | 1.477210974 | 0.004699527 | Late SSc |
| 76 | −1.400464213 | 1.471925259 | 0.005360609 | Late SSc |
| 77 | −1.400250828 | 1.476947968 | 0.005211465 | Late SSc |
| 78 | −1.399306124 | 1.495157038 | 0.004862558 | Late SSc |
| 79 | −1.39807413 | 1.380138694 | 0.00333459 | Late SSc |
| 80 | −1.396796568 | 1.382985423 | 0.003468855 | Late SSc |
| 81 | −1.393419416 | 1.400184107 | 0.003541756 | Late SSc |
| 82 | −1.392802437 | 1.480005974 | 0.005285174 | Late SSc |
| 83 | −1.391786866 | 1.413834843 | 0.003736567 | Late SSc |
| 84 | −1.391343564 | 1.409057391 | 0.003644071 | Late SSc |
| 85 | −1.389469621 | 1.498207138 | 0.005742942 | Late SSc |
| 86 | −1.388905146 | 1.429267903 | 0.003878624 | Late SSc |
| 87 | −1.387700446 | 1.473610575 | 0.005220571 | Late SSc |
| 88 | −1.382271601 | 1.462565011 | 0.004207329 | Late SSc |
| 89 | −1.382083594 | 1.454244682 | 0.004152482 | Late SSc |
| 90 | −1.381646553 | 1.467409322 | 0.005458532 | Late SSc |
| 91 | −1.381291697 | 1.461116048 | 0.00435845 | Late SSc |
| 92 | −1.380681871 | 1.45430681 | 0.00438966 | Late SSc |
| 93 | −1.380346255 | 1.457114043 | 0.004367161 | Late SSc |
| 94 | −1.380100261 | 1.462217536 | 0.004496421 | Late SSc |
| 95 | −1.379802039 | 1.466482296 | 0.004312114 | Late SSc |
| 96 | −1.379749126 | 1.501894245 | 0.00586349 | Late SSc |
| 97 | −1.379483586 | 1.501385344 | 0.006031711 | Late SSc |
| 98 | −1.379324736 | 1.50271561 | 0.006047702 | Late SSc |
| 99 | −1.378671665 | 1.496046282 | 0.005755859 | Late SSc |
| 100 | −1.378053209 | 1.518746877 | 0.006209069 | Late SSc |

TABLE 4.b3

| | |
|---|---|
| 51 | GATAGATTATGGAGGGTTAAGGAAGGAATCGATAACGCCAAGCACTCGGGCATGGGTGAC |
| 52 | AAAACCAGAGAAAGGAGCAAAGTGAGCCTCGAGTACAGAGGCACAGGGCAGCTTTTGGGG |
| 53 | ACTATTACTCAAATGTTAAAGCTCTTGGTCGAAGGGAGCATAATACAGGAAGGAGCCAGG |
| 54 | TTTCTACATTTTAGGGAGACATGAGATATCGAGACCAACCTGACAACATGGCAAAACCCC |
| 55 | AGTGGTATGACAATGGCTCACTGCAGCCTCGAACTCCACACCCCTGCTAGGAAGGGCACA |
| 56 | TAATTAGTGAGCCACAGACTCTAGGCCATCGAGGCTACAGTGAGCTATGATCACGTCACT |
| 57 | AGTTTCTAGAGAGGATTAAGGAGGGGGCTCGAGGGCCTTTGCCCTTTCGTAAGGGAAAGA |
| 58 | TTGGCTTCTGACTGTATACTCTGAGCACTCGAGGTGTACTGAGATATCTCGCCCCCGAGG |
| 59 | AAAAACCTTAGACTAGACGGATTCACAGTCGAGACAGTGTCTTACTCTGTTGTCCAGGCT |
| 60 | AGTGCAACAGACCAATGAAGTGAGACTTTCGATATAATAGTGGCAAACAAAATCCAGCAA |
| 61 | GGCAGAAAAATGAGTGAAGTCAGAGCGATCGAGTCTGCACCTATTTAATAATAACTCTCC |
| 62 | AAGCAGGTGTCCATTATTAATTCATTCATCGAACTCCTGGCCTTATGTAACCCACCTGCC |
| 63 | TCACTTAGTAGCCATCACGGTTATCAGATCGAACTCCTGTTCTCAAGTGATCTGCGTGCC |
| 64 | ATCTAGTTTCCATGCAAACTCAGAAGCATCGATTTGGGTGTGTCTGATGTATTTCCATGA |
| 65 | CCAGCAGTTCTGCAAAGACAAGCACTGGTCGATTGTGGATGGGAGTGTGGATTTTGTGGC |
| 66 | TTATGGGTAACTTCGTGTCTTTTTTTGTTCGAATTTTTACACCCGGCCCTTGCTTCCAGG |
| 67 | TTCAATTACCTCCCACTAGGTCCCTCCCTCGATAGCAGAATCTCCCAGTCTGGAGCACAG |
| 68 | TTCATAGTGCCTGGAATGTAGTAGGATCTCGAATCACTTCCTACCTGCAGGAGGCAGAGA |
| 69 | GGGTTTCCCCATGTTGGCCAGGTTGGTCTCGAGACCAGCCTGGCCAACATGGCAAAACCC |
| 70 | CGTTCCGGGAGTAAGTGAGCTTTGTGCTTCGAGCCTTCTTACTCAACCACTCAACATCTG |
| 71 | TTCCAAGGACACAACATGATCTCATCTTTCGAACTCCAGCCTGGGTAACAGAGACTCCAT |
| 72 | CTTCTATACGCCAGAACATGGATGAACCTCGAGATAATTTTGTATGTTTCCTTTTTTCTT |
| 73 | CCTGGCTCCTTCCTGTATTATGCTCCCTTCGAGAGAACACTATCAGAGCTCCCATGGCCC |
| 74 | TTCAAGTTCCCATCCAATGAGACATGTGTCGAGATCAGCCTGGCCAACGTGGTGAAACCC |
| 75 | AAATCTGGGGACCAGTAGCTGTGGCTAGTCGAGCCACTGCACTCCAGCCTGCAGGACAGA |
| 76 | TGTGTTCATTCAAGTCTTTGATCTTCACTCGAATCTGAGAGACAGAGAGAGAGCAAGGGA |
| 77 | AATCACTAAATTTCCAAATGTACATGGATCGACCTCCCAGGCTCAAGTGGTCTTCCTGTC |
| 78 | GAATGAAGGGGCCCTAGGGCAGTGGTGTTCGAGAGGCGGAAGTAAAAGGATTCAGATCCC |
| 79 | TTTGACTCTAGCCCAGTGATTCCCAAACTCGAGTCAAAGTTGAAAATTCATAGTAAGATT |
| 80 | AAAACAAATTGAGTATTTCAAGTAAACCTCGAACCACTGCACTCTAGTCCAGGCAACAGA |
| 81 | TGACTCACGAGGGAAGTGGTTGTTGACCTCGAGGAGACTGGACCAGAAGAGTTTCCCACC |
| 82 | GAGGTGGTGGGCAGGAAAGGAAGACAGTTCGAATGGTCTGTTCAAGACTATTTTATAGTG |
| 83 | GGCAGGAGGATTGCTTGAGCCTAGGAAATCGAACTCCTGGGCTCAAGCAATCCTCCCGCC |
| 84 | GGTTGGGGGATCACTTGAGGTCAGGAGCTCGAACTCCTGACCTCAAGTGATCCACTCTCC |
| 85 | CAGACTTTCATATTTTCAATCTCTCTCTTCGATGCTCAGGAAGCGCAGGTCCGTCAGCCT |
| 86 | AGGTTTCACCATGTTGGCCAGGCTGGTCTCGAGACCAGCCTGGCCAACATGGTGAAACCC |
| 87 | ATATTCCTCGCATGGAGGGAACTTGGGGTCGAAATCCTGGCCTCAAGCAATTCACCCACC |
| 88 | TAAGAAGTGGAAGTCGCCATGAGAGTGCTCGATACAATGGAATGTTATTCCACCATAAAA |
| 89 | GGTGGCTACATCATTATATGGCCAAGACTCGACTTTTAAATTCCTAAGTTTCCTAGTTGG |
| 90 | CGCGCACTGGATCAAAATAATACACGCCTCGAACTAACTTTCCAAGCTTGACTCTTAGGA |
| 91 | TTTACTGAACATTGGTAAATCTACCTAATCGAAATACAAAAATTAGCCAGGCATGATGGC |
| 92 | GTGATTGGTGAACAGTGGCTTCCTTTAGTCGACTAAATGGAACGTTTAAATGAGTTGAAG |
| 93 | CGCCTTTGAGTCTATTTCTGACTTCATCTCGACGGAATGTGGCCCACGGCATTAAATTTA |
| 94 | GCCAGGAGGATCATTTGAGGTCAGGAGTTCGAACTCCTGACCTCAGATGATCCACACACC |
| 95 | CCTGGCTCCTTCCTGTATTATGCTCCCTTCGAATCTTCATCGCTATCAGTCAGAAATTCT |
| 96 | ATGCCTGTAATCACGGGACTTTGGGAGGTCGATCTCATGACATTTAATATTACTTTTTGG |
| 97 | TTTGTTTCGGGTGCTCTAGAATATTCCATCGAGGGCCGTGGTTACAGAATCTTCTGGGGT |
| 98 | TCATGCAGAATTCCAATTAGCCTCTTAATCGAACTCCTGACCTCGGGGGATCGGCCCGCC |
| 99 | GAGCCGACCCTTGGGAGTCTGAACATCTTCGACTTCAAAAGCCACATAGATGATCCCCTT |
| 100 | TCTCCATAAAATATGGAGGATACTGCCCTCGAACTCCTGGGCTCCATTAATCTTCTGCCT |

TABLE 4.b4

| | | | | | |
|---|---|---|---|---|---|
| 51 | 12 | 51670783 | 51670812 | 51862307 | 51862336 |
| 52 | 7 | 137933935 | 137933964 | 137981880 | 137981909 |
| 53 | 11 | 61044371 | 61044400 | 61245995 | 61246024 |
| 54 | X | 2888193 | 2888222 | 3049156 | 3049185 |
| 55 | 11 | 115618314 | 115618343 | 115715127 | 115715156 |
| 56 | X | 77646857 | 77646886 | 77916549 | 77916578 |
| 57 | 20 | 62658424 | 62658453 | 62935925 | 62935954 |
| 58 | 22 | 17927992 | 17928021 | 18001574 | 18001603 |
| 59 | 19 | 47777296 | 47777325 | 47959276 | 47959305 |
| 60 | 8 | 7883774 | 7883803 | 7966331 | 7966360 |
| 61 | 11 | 12094327 | 12094356 | 12153673 | 12153702 |
| 62 | 17 | 31222341 | 31222370 | 31324783 | 31324812 |
| 63 | 7 | 4621704 | 4621733 | 4724417 | 4724446 |
| 64 | 10 | 45820550 | 45820579 | 45903868 | 45903897 |
| 65 | 16 | 81474881 | 81474910 | 81607036 | 81607065 |
| 66 | 11 | 10779744 | 10779773 | 10903577 | 10903606 |
| 67 | 8 | 97253468 | 97253497 | 97357381 | 97357410 |
| 68 | 1 | 109683571 | 109683600 | 109741803 | 109741832 |
| 69 | 1 | 7776999 | 7777028 | 7968705 | 7968734 |
| 70 | 12 | 7858283 | 7858312 | 7933480 | 7933509 |
| 71 | 13 | 30408293 | 30408322 | 30585634 | 30585663 |
| 72 | 15 | 30290702 | 30290731 | 30544057 | 30544086 |
| 73 | 11 | 61152529 | 61152558 | 61245995 | 61246024 |
| 74 | 17 | 64322185 | 64322214 | 64385556 | 64385585 |
| 75 | 6 | 26459327 | 26459356 | 26493176 | 26493205 |
| 76 | 2 | 74507106 | 74507135 | 74521816 | 74521845 |
| 77 | 2 | 159238608 | 159238637 | 159255031 | 159255060 |
| 78 | 16 | 46904051 | 46904080 | 47061438 | 47061467 |
| 79 | 5 | 40613214 | 40613243 | 40692973 | 40693002 |
| 80 | 4 | 47912106 | 47912135 | 48015634 | 48015663 |
| 81 | 4 | 38166333 | 38166362 | 38180540 | 38180569 |
| 82 | 7 | 22482165 | 22482194 | 22587352 | 22587381 |
| 83 | 17 | 27477799 | 27477828 | 27629085 | 27629114 |
| 84 | 10 | 13712288 | 13712317 | 13854653 | 13854682 |
| 85 | 15 | 79006297 | 79006326 | 79127310 | 79127339 |
| 86 | 19 | 9779760 | 9779789 | 9833968 | 9833997 |
| 87 | 17 | 48578249 | 48578278 | 48766630 | 48766659 |
| 88 | 3 | 124942090 | 124942119 | 125145444 | 125145473 |
| 89 | 2 | 230319554 | 230319583 | 230433768 | 230433797 |
| 90 | 5 | 179455076 | 179455105 | 179634355 | 179634384 |
| 91 | 13 | 94397469 | 94397498 | 94478317 | 94478346 |
| 92 | 1 | 185486627 | 185486656 | 185571368 | 185571397 |
| 93 | 7 | 105801950 | 105801979 | 105905226 | 105905255 |
| 94 | 17 | 75411074 | 75411103 | 75651388 | 75651417 |
| 95 | 11 | 61227169 | 61227198 | 61379384 | 61379413 |
| 96 | 5 | 94427268 | 94427297 | 94505844 | 94505873 |
| 97 | 17 | 82440613 | 82440642 | 82511427 | 82511456 |
| 98 | 17 | 59895956 | 59895985 | 59916321 | 59916350 |
| 99 | 2 | 102051984 | 102052013 | 102268038 | 102268067 |
| 100 | 1 | 203291123 | 203291152 | 203473867 | 203473896 |

TABLE 4.b5

| | | | | | | |
|---|---|---|---|---|---|---|
| 51 | 12 | 51666813 | 51670812 | 51862307 | 51866306 | ORF1_12_51666821_51670814_51862305_51869704_FR |
| 52 | 7 | 137929965 | 137933964 | 137981880 | 137985879 | ORF1_7_137931629_137933966_137981878_137983291_FR |
| 53 | 11 | 61040401 | 61044400 | 61242025 | 61246024 | ORF1_11_61042263_61044402_61243450_61246026_FF |
| 54 | X | 2888193 | 2892192 | 3045186 | 3049185 | ORF1_X_2888191_2891631_3046105_3049187_RF |
| 55 | 11 | 115614344 | 115618343 | 115711157 | 115715156 | ORF1_11_115616166_115618345_115711125_115715158_FF |
| 56 | X | 77646857 | 77650856 | 77912579 | 77916578 | ORF1_X_77646855_77652042_77915112_77916580_RF |
| 57 | 20 | 62658424 | 62662423 | 62931955 | 62935954 | ORF1_20_62658422_62663845_62934812_62935956_RF |
| 58 | 22 | 17924022 | 17928021 | 17997604 | 18001603 | ORF1_22_17921708_17928023_17999187_18001605_FF |
| 59 | 19 | 47777296 | 47781295 | 47955306 | 47959305 | ORF1_19_47777294_47778517_47957709_47959307_RF |
| 60 | 8 | 7879804 | 7883803 | 7962361 | 7966360 | ORF1_8_7882531_7883805_7954782_7966362_FF |
| 61 | 11 | 12090357 | 12094356 | 12153673 | 12157672 | ORF1_11_12091217_12094358_12153671_12160688_FR |
| 62 | 17 | 31222341 | 31226340 | 31324783 | 31328782 | ORF1_17_31222339_31224131_31324781_31330350_RR |
| 63 | 7 | 4617734 | 4621733 | 4724417 | 4728416 | ORF1_7_4620261_4621735_4724415_4726417_FR |
| 64 | 10 | 45820550 | 45824549 | 45899898 | 45903897 | ORF1_10_45820548_45823847_45898343_45903899_RF |
| 65 | 16 | 81474881 | 81478880 | 81607036 | 81611035 | ORF1_16_81474879_81476453_81607034_81611259_RR |
| 66 | 11 | 10775774 | 10779773 | 10899607 | 10903606 | ORF1_11_10776569_10779775_10900691_10903608_FF |
| 67 | 8 | 97253468 | 97257467 | 97353411 | 97357410 | ORF1_8_97253466_97261198_97345458_97357412_RF |
| 68 | 1 | 109679601 | 109683600 | 109741803 | 109745802 | ORF1_1_109681178_109683602_109741801_109745521_FR |
| 69 | 1 | 7773029 | 7777028 | 7964735 | 7968734 | ORF1_1_7774817_7777030_7966809_7968736_FF |
| 70 | 12 | 7858283 | 7862282 | 7929510 | 7933509 | ORF1_12_7858281_7862390_7932397_7933511_RF |
| 71 | 13 | 30408293 | 30412292 | 30585634 | 30589633 | ORF1_13_30408291_30410407_30585632_30589891_RR |
| 72 | 15 | 30286732 | 30290731 | 30544057 | 30548056 | ORF1_15_30288521_30290733_30544055_30547496_FR |
| 73 | 11 | 61152529 | 61156528 | 61242025 | 61246024 | ORF1_11_61152527_61154904_61243450_61246026_RF |
| 74 | 17 | 64322185 | 64326184 | 64381586 | 64385585 | ORF1_17_64322183_64329531_64384310_64385587_RF |
| 75 | 6 | 26459327 | 26463326 | 26493176 | 26497175 | ORF1_6_26459325_26460705_26493174_26499033_RR |
| 76 | 2 | 74503136 | 74507135 | 74517846 | 74521845 | ORF1_2_74503923_74507137_74519716_74521847_FF |
| 77 | 2 | 159234638 | 159238637 | 159251061 | 159255060 | ORF1_2_159230477_159238639_159250572_159255062_FF |
| 78 | 16 | 46900081 | 46904080 | 47057468 | 47061467 | ORF1_16_46899253_46904082_47060044_47061469_FF |
| 79 | 5 | 40609244 | 40613243 | 40692973 | 40696972 | ORF1_5_40609009_40613245_40692971_40695207_FR |
| 80 | 4 | 47908136 | 47912135 | 48011664 | 48015663 | ORF1_4_47908113_47912137_48013964_48015665_FF |
| 81 | 4 | 38162363 | 38166362 | 38180540 | 38184539 | ORF1_4_38161792_38166364_38180538_38182770_FR |
| 82 | 7 | 22482165 | 22486164 | 22587352 | 22591351 | ORF1_7_22482163_22488443_22587350_22592472_RR |
| 83 | 17 | 27477799 | 27481798 | 27629085 | 27633084 | ORF1_17_27477797_27480875_27629083_27630392_RR |
| 84 | 10 | 13708318 | 13712317 | 13854653 | 13858652 | ORF1_10_13707193_13712319_13854651_13857653_FR |
| 85 | 15 | 79006297 | 79010296 | 79123340 | 79127339 | ORF1_15_79006295_79007772_79123113_79127341_RF |
| 86 | 19 | 9775790 | 9779789 | 9829998 | 9833997 | ORF1_19_9777468_9779791_9828343_9833999_FF |
| 87 | 17 | 48574279 | 48578278 | 48762660 | 48766659 | ORF1_17_48576277_48578280_48760984_48766661_FF |
| 88 | 3 | 124942090 | 124946089 | 125145444 | 125149443 | ORF1_3_124942088_124944537_125145442_125149201_RR |
| 89 | 2 | 230319554 | 230323553 | 230433768 | 230437767 | ORF1_2_230319552_230322976_230433766_230436650_RR |
| 90 | 5 | 179455076 | 179459075 | 179630385 | 179634384 | ORF1_5_179455074_179456297_179632102_179634386_RF |
| 91 | 13 | 94393499 | 94397498 | 94478317 | 94482316 | ORF1_13_94395452_94397500_94478315_94481582_FR |
| 92 | 1 | 185482657 | 185486656 | 185567398 | 185571397 | ORF1_1_185482953_185486658_185569901_185571399_FF |
| 93 | 7 | 105801950 | 105805949 | 105901256 | 105905255 | ORF1_7_105801948_105809991_105902395_105905257_RF |
| 94 | 17 | 75411074 | 75415073 | 75647418 | 75651417 | ORF1_17_75411072_75413612_75649583_75651419_RF |
| 95 | 11 | 61223199 | 61227198 | 61375414 | 61379413 | ORF1_11_61224624_61227200_61378098_61379415_FF |
| 96 | 5 | 94423298 | 94427297 | 94505844 | 94509843 | ORF1_5_94419239_94427299_94505842_94514405_FR |
| 97 | 17 | 82436643 | 82440642 | 82511427 | 82515426 | ORF1_17_82434207_82440644_82511425_82512972_FR |
| 98 | 17 | 59891986 | 59895985 | 59916321 | 59920320 | ORF1_17_59893718_59895987_59916319_59918152_FR |
| 99 | 2 | 102051984 | 102055983 | 102268038 | 102272037 | ORF1_2_102051982_102057593_102268036_102274328_RR |
| 100 | 1 | 203287153 | 203291152 | 203473867 | 203477866 | ORF1_1_203288566_203291154_203473865_203477190_FR |

TABLE 4.b6

| | | | | |
|---|---|---|---|---|
| 51 | OBD168-1073 | CTGAGTTACAGTGGTGGGAGTGG | OBD168-1075 | CTTGCTCTTGTCCACCACGCCGT |
| 52 | OBD168-873 | GTCAACATCCCATTGCTGGTGCC | OBD168-875 | CTCCCTCTTGCTGCTGGGCACTA |
| 53 | OBD168-1769 | TTGCGTTATAAAAACATGTATAAAA | OBD168-1771 | CTGGCATCCAGAGCTCAGTGAACAT |
| 54 | OBD168-1709 | AATATGAGTGACCATGGCCGC | OBD168-1711 | ACGATCTCGGCTTACTGCAAC |
| 55 | OBD168-1785 | GGATGCATCCCAAGGGCCTATTTC | OBD168-1787 | CATTTTTCCGGGGAATTTTTGTGC |
| 56 | OBD168-2113 | ATAGTACACCTACATATGCTAA | OBD168-2115 | CTTTATATTTATTAAGGCTTTT |
| 57 | OBD168-1581 | CCCTGTCTGCTATGTGAGAGGTAGTG | OBD168-1583 | AGGGGCTGCGGAGGGCCGTGTGGGGC |
| 58 | OBD168-933 | CGCCAGAACCACAACACACACTC | OBD168-935 | TGCCTCATACTGCGGTGTCCCTC |
| 59 | OBD168-1501 | TGGAAACATAAAACCTCTCAAGA | OBD168-1503 | ATGTACCTGTACTCCCAGTTACT |
| 60 | OBD168-2077 | CTTTCCATCCAAAATGGACTCACTGC | OBD168-2079 | GGTTTTTAATTTAAAGGAATGTTGAA |
| 61 | OBD168-937 | GGAGGGCAGCATTCACACTGGAC | OBD168-939 | GGGTGCCCAGGAAAACTGTGAAC |
| 62 | OBD168-1025 | CATTTTCCTCCGTGTGGTGACTTCAT | OBD168-1027 | AAATGGGAGCAGCCACAGTGAGGAC |
| 63 | OBD168-1237 | TTTGTCCAGGGAACCCACACTGC | OBD168-1239 | CGAGGCACAGACCCCTGATTGAC |
| 64 | OBD168-1345 | GGCTCTTCTGTGAGGCACTCTGCCA | OBD168-1347 | AGAACTATCAATGCCATAACTCTGA |
| 65 | OBD168-1277 | CCTCCAGACACAGCATCTTCTCC | OBD168-1279 | CCCCACCCTCCTCCTCAACATTC |
| 66 | OBD168-1757 | TATAATGTGTTAGGTTGAATAAG | OBD168-1759 | AGAAAGAAGAAGAAGCTGATAGT |
| 67 | OBD168-721 | GAAGGAGGAACTGTCAAACACTCACA | OBD168-723 | CCTGGCTATGTAAGTCCTCTGCTGTG |
| 68 | OBD168-1041 | CTCTCCCCTGAAGTTCCCACCTG | OBD168-1043 | CATCTCTGCTACTCGTGCTCCCA |
| 69 | OBD168-1301 | CTCCTGAGTAGTTGGGATTACA | OBD168-1303 | TGCCTCCTAGGTTCAAGCGATT |
| 70 | OBD168-1797 | TCTGCTAAGGGAACCCAGCTTC | OBD168-1799 | AAAGTGAAAAGACAACCCACAG |
| 71 | OBD168-565 | CTGGAGAACTGAGCAGAGCGGGC | OBD168-567 | CCCCTTAGTCTGGGATTGTAGATTG |
| 72 | OBD168-1837 | GATTAGAAATACAGGGGACTCCA | OBD168-1839 | AACACTAAAATTTCTCTAAGAGA |
| 73 | OBD168-749 | GGTTGGAGGCATCTCTGACACCA | OBD168-751 | GCGGAGAAGCCTGTGCTGCGAAT |
| 74 | OBD168-1461 | GGCTGCCCACCCTCGTCAGGAAAA | OBD168-1463 | CCTGGGTTCAAGCAGTTCTCCTGT |
| 75 | OBD168-1029 | CCATAGGGTCTCTTTGGGCTGAG | OBD168-1031 | CTGGGAGTGCCTGACCTGTTCTC |
| 76 | OBD168-1545 | TCTCCAAGGCACTCACAAGTAT | OBD168-1547 | GAAACGTTCATCAGGAGTTGGA |
| 77 | OBD168-1929 | CTGGGTTTCTGATTCATTTGG | OBD168-1931 | ACACTTTGGGAGGCCAAGGCT |
| 78 | OBD168-1413 | AGGGGAAACGTGGCATTCACG | OBD168-1415 | GTGTGAGCTGCTCCCTCTACC |
| 79 | OBD168-1625 | ACAGAAAAGAGAGAGGAATGGCA | OBD168-1627 | GTTTTCTGGATAAATAAATGTAA |
| 80 | OBD168-641 | GGACCACAGGGATTTTCTTCAAGCAC | OBD168-643 | CACTACCCCACCCCTCACCTTTT |
| 81 | OBD168-1617 | CTCCCTGAAATGCAGATGGAAATG | OBD168-1619 | AAATTGATACTTTCCACAACAATG |
| 82 | OBD168-685 | GCTCTGCTTCTGGGAACCCTTTC | OBD168-687 | CAGGAAGCCCAGATGCTGTGTGG |
| 83 | OBD168-1157 | GGTGTGGTGCCTCACGCCTTGTA | OBD168-1159 | GCAGGAAGGAGTCTTTGAGAGGC |
| 84 | OBD168-561 | CCTTGGAGTCCTGGCTTCCTCTA | OBD168-563 | GCAGTTCAGCCCACAGACAGCATC |
| 85 | OBD168-753 | CTGCTTGCTTGGGAGTCTGTGGC | OBD168-755 | TCTCAGGTCCGACGCCTCCTTAT |
| 86 | OBD168-1893 | CAAGTTTCACTCTTGTTGCCCAGG | OBD168-1895 | GTCTCGCTCTGTCACCTACGCTGG |
| 87 | OBD168-1453 | CCGCGTAGCGCTGCACGGTGCA | OBD168-1455 | CTTCCCTTTCTTCGTGTGGCTT |
| 88 | OBD168-1985 | GGCGACAGAGTGAGACCCTGTCT | OBD168-1987 | GCCCTGTGGATAAAGGGGAGCTA |
| 89 | OBD168-841 | AGACACCAGAGAGAGAGCCAGAAGTG | OBD168-843 | GCTTAGTGGACAGTTTCAGGGTTCTA |
| 90 | OBD168-1129 | AAGCCAAGCCAAGTCACATACGGCAC | OBD168-1131 | CTCCTGTCAGTAATCCCATACATTGG |
| 91 | OBD168-1821 | AGGAGTCAGAGAGGGCCCCAAGCACA | OBD168-1823 | GTGGCTGGAGTGCAATGGTGCTATCT |
| 92 | OBD168-1333 | CGTGTGCTAGAGAAGGTGACAGGTCA | OBD168-1335 | CAGTTATTCATTCACCAGAAATGTGT |
| 93 | OBD168-981 | CAAATCTCTTCTTGCTCAACCGCAGC | OBD168-983 | CTTAGGAAGGTTTTCAGACACCAGGC |
| 94 | OBD168-1469 | TGGACTACCTTAGAGCCACAAAA | OBD168-1471 | TGTAACCCCAGCACTTTAGGATG |
| 95 | OBD168-1773 | GGCTCTGGGCCTGAGGCTGGC | OBD168-1775 | TGGTGGTGCGTGTCTGTAATC |
| 96 | OBD168-1633 | CAGTCTTTTTATCACTCAATTTGT | OBD168-1635 | TGATTGCTGATAGCTAAACGTATT |
| 97 | OBD168-1473 | CCCATCCGCCACCCGTGACTGGGGCA | OBD168-1475 | TGCAAACCCTTCCCTGCTTTCTGCGT |
| 98 | OBD168-1457 | GCTAATTGTTTGTATTTTAGT | OBD168-1459 | GTAGATTCAAAGAAAGCAAAA |
| 99 | OBD168-1181 | TACTCGCCAGGCAGTGATTCCAC | OBD168-1183 | GAGGGCACAGGACAGAGGTCACA |
| 100 | OBD168-809 | GACATCCTTGCCTCTGCCTTGGG | OBD168-811 | GTGGGTGGTAGATGATAGAGGGC |

TABLE 4.b7

| | | | |
|---|---|---|---|
| 51 | OBD168-q1073 | CTGAGGAACAGTGGAAGATGAGGCTGGG | OBD168-q1075 |
| 52 | OBD168-q873 | GGGTGTCCTTGGCAGTGGTGGGC | OBD168-q875 |
| 53 | OBD168-q1769 | TTAAATATTCTTAGAAAAAAGTT | OBD168-q1771 |
| 54 | OBD168-q1709 | CAAATATGAGTGACCATGGCCGC | OBD168-q1711 |
| 55 | OBD168-q1785 | AAGGGCCTATTTCCTCGTTCTAG | OBD168-q1787 |
| 56 | OBD168-q2113 | CATTATTACAGCTGGCAAATCAAAT | OBD168-q2115 |
| 57 | OBD168-q1581 | TGAGAGGTAGTGACAGGACTT | OBD168-q1583 |
| 58 | OBD168-q933 | CTCTGTTCTCCAAGCACCTCCCACCAGG | OBD168-q935 |
| 59 | OBD168-q1501 | AAGAAATGGAAACCCTGAACAAACCA | OBD168-q1503 |
| 60 | OBD168-q2077 | CTGCTCCTCACTTTCCATCCAAAATG | OBD168-q2079 |
| 61 | OBD168-q937 | GCCAGACACAGAGGATGAGGAGAGCAGC | OBD168-q939 |
| 62 | OBD168-q1025 | TACATTTTCCTCCGTGTGGTGACTTCAT | OBD168-q1027 |
| 63 | OBD168-q1237 | GCAACACTCGGCTCCATCCCACTTGG | OBD168-q1239 |
| 64 | OBD168-q1345 | AAATACGAGCAAGTGATAGTCCGG | OBD168-q1347 |
| 65 | OBD168-q1277 | GAGGGAGGGAGAGAAGGGAGGAAAGGGT | OBD168-q1279 |
| 66 | OBD168-q1757 | ATGTAGATAAAATCACAAATG | OBD168-q1759 |
| 67 | OBD168-q721 | CGCAACCAGGAGTCTGCCTACAT | OBD168-q723 |
| 68 | OBD168-q1041 | ACCCCTCTCCCACTCCACTGCCTCTCAG | OBD168-q1043 |
| 69 | OBD168-q1301 | CTCAGCCTCCTGAGTAGTTGGGAT | OBD168-q1303 |
| 70 | OBD168-q1797 | GCACCGATGTTCACAGTCTACCC | OBD168-q1799 |
| 71 | OBD168-q565 | CGCCCTGGAGAACTGAGCAGAGCGGG | OBD168-q567 |
| 72 | OBD168-q1837 | ACTCCATGTTCATAGTGGCGTTATTC | OBD168-q1839 |
| 73 | OBD168-q749 | CCCACCCAGGTTGGAGGCATCTCTGAC | OBD168-q751 |
| 74 | OBD168-q1461 | TGGAGGGTGCAGGAGAAGGAGGGGCT | OBD168-q1463 |
| 75 | OBD168-q1029 | GCCACGCTGCCCCTGCTGATGTCTTTGC | OBD168-q1031 |
| 76 | OBD168-q1545 | GGGTGGGGTGCCTGGATGCGTGAGG | OBD168-q1547 |
| 77 | OBD168-q1929 | CCCTTTTTATGTCTGGGTTTCTGA | OBD168-q1931 |
| 78 | OBD168-q1413 | GTAAGAACCTGCAAATGCATGCC | OBD168-q1415 |
| 79 | OBD168-q1625 | AATCTAGGGTGTAAATAATAC | OBD168-q1627 |
| 80 | OBD168-q641 | GCAAGGACCACAGGGATTTTCTTCAAGC | OBD168-q643 |
| 81 | OBD168-q1617 | GTGCTGCCATCCGCAGGCTTAAA | OBD168-q1619 |
| 82 | OBD168-q685 | CCTCTCTGCCTATCCTTCTTCCCAGGGC | OBD168-q687 |
| 83 | OBD168-q1157 | GCCAGGTGTGGTGCCTCACGCCTTGTA | OBD168-q1159 |
| 84 | OBD168-q561 | CTTCTATCCTTGGAGTCCTGGCTTCCTC | OBD168-q563 |
| 85 | OBD168-q753 | GACCTGCTTGCTTGGGAGTCTGTGGC | OBD168-q755 |
| 86 | OBD168-q1893 | TTCTGCCTCCCAGGTTCAAGGAATT | OBD168-q1895 |
| 87 | OBD168-q1453 | GCGGGGGCCCAGGGTCCCGGCAG | OBD168-q1455 |
| 88 | OBD168-q1985 | TGAGCAGAGATTGCACCACTG | OBD168-q1987 |
| 89 | OBD168-q841 | CAGACACCAGAGAGAGAGCCAGAAGTGG | OBD168-q843 |
| 90 | OBD168-q1129 | TTCCATCCCGCACAGGTGAGCCGTCTGG | OBD168-q1131 |
| 91 | OBD168-q1821 | CCTAGGCAGCTTAGGCAGCTCACAT | OBD168-q1823 |
| 92 | OBD168-q1333 | GGTGACAGGTCATGAGGAAGAGA | OBD168-q1335 |
| 93 | OBD168-q981 | GGGCACAGTCATAAGCCAGGAAATCCCT | OBD168-q983 |
| 94 | OBD168-q1469 | GAATTGGACTACCTTAGAGCCACA | OBD168-q1471 |
| 95 | OBD168-q1773 | CCAAGTCCTGCATGAGATGAACCAGG | OBD168-q1775 |
| 96 | OBD168-q1633 | AAATAGCTAATACTAAATAAGGACAT | OBD168-q1635 |
| 97 | OBD168-q1473 | CTTGCCCACGGCCCCATCCGC | OBD168-q1475 |
| 98 | OBD168-q1457 | TTTTAGTAGAGATGGGGTTTCAC | OBD168-q1459 |
| 99 | OBD168-q1181 | GGCATTGACCTCATACTCGCCAGGCAG | OBD168-q1183 |
| 100 | OBD168-q809 | TCAAGAGCCAGAGCCCAGGCGGG | OBD168-q811 |

TABLE 4.b8

| | | | |
|---|---|---|---|
| 51 | GAACGCTTGCTCTTGTCCACCACGCCG | OBD168-p1073 | AGGAAGGAATCGATAACGCCAAGCACTCGGGC |
| 52 | GCTCTGCTCCCTCTTGCTGCTGGGCACT | OBD168-p873 | AGGAGCAAAGTGAGCCTCGAGTACAGAGGCAC |
| 53 | CTGAGGCTGGCATCCAGAGCTCA | OBD168-p1769 | AAATGTTAAAGCTCTTGGTCGAAGGGAGCATAATACAGGA |
| 54 | CCGCCTCCAAGGTTCAAGCGATT | OBD168-p1709 | TTAGGGAGACATGAGATATCGAGACCAACCTGACAACATG |
| 55 | ATTTTTCCGGGGAATTTTTGTGC | OBD168-p1785 | CAATGGCTCACTGCAGCCTCGAACTCCACACCCCTGCTAG |
| 56 | AGGCTTTTTTGTGCCTGATTATATG | OBD168-p2113 | GCCACAGACTCTAGGCCATCGAGGCTACAGTGAGCTATGA |
| 57 | CTCCGGCCCACTCCCCTCATG | OBD168-p1581 | GAGGATTAAGGAGGGGGCTCGAGGGCCTTTGCCCTTTCGT |
| 58 | TCTGCCTCATACTGCGGTGTCCCTCGG | OBD168-p933 | TGGCTTCTGACTGTATACTCTGAGCACTCGAGGTG |
| 59 | CCAAGAGTCTGAGACCAGCTGGGGCA | OBD168-p1501 | GACTAGACGGATTCACAGTCGAGACAGTGTCTTACTCTGT |
| 60 | TCAAAGATCTATTCTGAGTCTACTGA | OBD168-p2077 | ACCAATGAAGTGAGACTTTCGATATAATAGTGGCAAACAA |
| 61 | CCCCTGCTGGTGAAGTCCCCAAGAAGG | OBD168-p937 | ATGAGTGAAGTCAGAGCGATCGAGTCTGCACC |
| 62 | TAAATGGGAGCAGCCACAGTGAGGACCA | OBD168-p1025 | TCATTCATCGAACTCCTGGCCTTATGTAACCCACCTG |
| 63 | CCTCCCTCCTCTCTCCCCTCTGGAATGG | OBD168-p1237 | AGTAGCCATCACGGTTATCAGATCGAACTCCTGTTCTC |
| 64 | TACAATGGAGAAATCCAGCAGAAA | OBD168-p1345 | CATGCAAACTCAGAAGCATCGATTTGGGTGTGTCTGATGT |
| 65 | GCTGGAGGGCAAGAACTGAGGGACTGC | OBD168-p1277 | ACAAGCACTGGTCGATTGTGGATGGGAGTGTGG |
| 66 | TCCTTGAGGGAAAAGTCACTG | OBD168-p1757 | CTTCGTGTCTTTTTTTGTTCGAATTTTTACACCCGGCCCT |
| 67 | TCCCTGGCTATGTAAGTCCTCTGCTGTG | OBD168-p721 | AATTACCTCCCACTAGGTCCCTCCCTCGATAGC |
| 68 | GCCTCGGGCAGGTCTCTTGCTCTG | OBD168-p1041 | AGTGCCTGGAATGTAGTAGGATCTCGAATCACTTCC |
| 69 | CTAGAGTGCGGTGGTGCAGCCTCA | OBD168-p1301 | ATGTTGGCCAGGTTGGTCTCGAGACCAGCCTGGCCAACAT |
| 70 | TGTGTATGATAAGGGGCTTGTAT | OBD168-p1797 | GTAAGTGAGCTTTGTGCTTCGAGCCTTCTTACTCAACCAC |
| 71 | GGGTTGGAGACCACAGTGACAGGACAGT | OBD168-p565 | TCTCATCTTTCGAACTCCAGCCTGGGTAACAGAGAC |
| 72 | TTCTCTAAGAGAACCTTCTACTTTCT | OBD168-p1837 | CCAGAACATGGATGAACCTCGAGATAATTTTGTATGTTTC |
| 73 | GGAGAGCAAGAGCAAAGGCGGAGAAGCC | OBD168-p749 | TGCTCCCTTCGAGAGAACACTATCAGAGCTCCC |
| 74 | GCTGGACTGCAGCGGCGCAACCTCTG | OBD168-p1461 | CATCCAATGAGACATGTGTCGAGATCAGCCTGGCCAACGT |
| 75 | GCTGGGAGTGCCTGACCTGTTCTCA | OBD168-p1029 | TGGCTAGTCGAGCCACTGCACTCCAGCCTG |
| 76 | CTCTCTCTCCCTCTCTTTCCCTCTC | OBD168-p1545 | CAAGTCTTTGATCTTCACTCGAATCTGAGAGACAGAGAGA |
| 77 | TGCAGTGGCTCATGCCCATAATCC | OBD168-p1929 | TTTCCAAATGTACATGGATCGACCTCCCAGGCTCAAGTGG |
| 78 | TCCAGGCTGTGTGGGATCCTTGG | OBD168-p1413 | GCCCTAGGGCAGTGGTGTTCGAGAGGCGGAAGTAAAAGGA |
| 79 | TAAATAAATGTAATATCACTA | OBD168-p1625 | GCCCAGTGATTCCCAAACTCGAGTCAAAGTTGAAAATTCA |
| 80 | GAGCACAAAGGCACCCCAGTTCTCCCTG | OBD168-p641 | AGTAAACCTCGAACCACTGCACTCTAGTCCAGGC |
| 81 | TTGTAAATTGATACTTTCCACAA | OBD168-p1617 | GGGAAGTGGTTGTTGACCTCGAGGAGACTGGACCAGAAGA |
| 82 | CAGGAAGCCCAGATGCTGTGTGGTTTT | OBD168-p685 | TGGTGGGCAGGAAAGGAAGACAGTTCGAATGGTC |
| 83 | GGGCAGGAAGGAGTCTTTGAGAGGCTGG | OBD168-p1157 | TGCTTGAGCCTAGGAAATCGAACTCCTGGGCTC |
| 84 | GCAGTTCAGCCCACAGACAGCATCTTCT | OBD168-p561 | AGGAGCTCGAACTCCTGACCTCAAGTGATCCAC |
| 85 | CTCCCCTCTCAGGTCCGACGCCTCCTTA | OBD168-p753 | TCTCTCTCTTCGATGCTCAGGAAGCGCAGGTC |
| 86 | TGGAGTGCAGTGGTGCGATCTCGGC | OBD168-p1893 | ATGTTGGCCAGGCTGGTCTCGAGACCAGCCTGGCCAACAT |
| 87 | CTTCCCTTTCTTCGTGTGGCTTC | OBD168-p1453 | CATGGAGGGAACTTGGGGTCGAAATCCTGGCCTCAAGCAA |
| 88 | ATTATCTGCCATTTCAGGCCT | OBD168-p1985 | AAGTCGCCATGAGAGTGCTCGATACAATGGAATGTTATTC |
| 89 | CTGGCTTAGTGGACAGTTTCAGGGTTCT | OBD168-p841 | TGGTGGCTACATCATTATATGGCCAAGACTCGACTTT |
| 90 | GCTCAACCCGTGACCACAGCATCTGC | OBD168-p1129 | ACGCGCACTGGATCAAAATAATACACGCCTCGAACTA |
| 91 | TTGGAGATGGAGTCTCGCTTTGTCA | OBD168-p1821 | ATTGGTAAATCTACCTAATCGAAATACAAAAATTAGCCAG |
| 92 | GGGGAAAAAAAGGCTGTATTCTG | OBD168-p1333 | AACAGTGGCTTCCTTTAGTCGACTAAATGGAACGTTTAAA |
| 93 | GGCAGCCCTCGGGAGAACAGGTTGC | OBD168-p981 | TCTGACTTCATCTCGACGGAATGTGGCCCACG |
| 94 | ACTTTAGGATGCTGAGGTGGGAGG | OBD168-p1469 | TCATTTGAGGTCAGGAGTTCGAACTCCTGACCTCAGATGA |
| 95 | ACTTCAACCCAGGAGGAGGAGGTTGC | OBD168-p1773 | TCCTGTATTATGCTCCCTTCGAATCTTCATCGCTATCAGT |
| 96 | TACCTGGGGACAATAGTCAATAGATT | OBD168-p1633 | TCACGGGACTTTGGGAGGTCGATCTCATGACATTTAATAT |
| 97 | TACTCCCTTTGCAAACCCTTC | OBD168-p1473 | GTGCTCTAGAATATTCCATCGAGGGCCGTGGTTACAGAAT |
| 98 | CAAAACAAAATGAGAGGATGAGG | OBD168-p1457 | TTCCAATTAGCCTCTTAATCGAACTCCTGACCTCGGGGGA |
| 99 | GGCGGAGGGCACAGGACAGAGGTCACAT | OBD168-p1181 | ACCCTTGGGAGTCTGAACATCTTCGACTTCAAAAGCC |
| 100 | CGCCCTGCTGGTGAGGTCTTGCTGGAAG | OBD168-p809 | ATATGGAGGATACTGCCCTCGAACTCCTGGGC |

TABLE 4.b9

| | | |
|---|---|---|
| 51 | OBD168-p1075 | TGCCCGAGTGCTTGGCGTTATCGATTCCTTCC |
| 52 | OBD168-p875 | TGTGCCTCTGTACTCGAGGCTCACTTTGCTCC |
| 53 | OBD168-p1771 | TCCTGTATTATGCTCCCTTCGACCAAGAGCTTTAACATTT |
| 54 | OBD168-p1711 | CATGTTGTCAGGTTGGTCTCGATATCTCATGTCTCCCTAA |
| 55 | OBD168-p1787 | CTAGCAGGGGTGTGGAGTTCGAGGCTGCAGTGAGCCATTG |
| 56 | OBD168-p2115 | TCATAGCTCACTGTAGCCTCGATGGCCTAGAGTCTGTGGC |
| 57 | OBD168-p1583 | ACGAAAGGGCAAAGGCCCTCGAGCCCCCTCCTTAATCCTC |
| 58 | OBD168-p935 | ACACCTCGAGTGCTCAGAGTATACAGTCAGAAGCC |
| 59 | OBD168-p1503 | ACAGAGTAAGACACTGTCTCGACTGTGAATCCGTCTAGTC |
| 60 | OBD168-p2079 | TTGTTTGCCACTATTATATCGAAAGTCTCACTTCATTGGT |
| 61 | OBD168-p939 | AGGTGCAGACTCGATCGCTCTGACTTCACTCA |
| 62 | OBD168-p1027 | AGGTGGGTTACATAAGGCCAGGAGTTCGATGAATG |
| 63 | OBD168-p1239 | TGAGAACAGGAGTTCGATCTGATAACCGTGATGGCTAC |
| 64 | OBD168-p1347 | ACATCAGACACACCCAAATCGATGCTTCTGAGTTTGCATG |
| 65 | OBD168-p1279 | TCCACACTCCCATCCACAATCGACCAGTGCTTG |
| 66 | OBD168-p1759 | AGGGCCGGGTGTAAAAATTCGAACAAAAAAAGACACGAAG |
| 67 | OBD168-p723 | TGCTATCGAGGGAGGGACCTAGTGGGAGGTAA |
| 68 | OBD168-p1043 | AGGAAGTGATTCGAGATCCTACTACATTCCAGGCAC |
| 69 | OBD168-p1303 | ATGTTGGCCAGGCTGGTCTCGAGACCAACCTGGCCAACAT |
| 70 | OBD168-p1799 | GTGGTTGAGTAAGAAGGCTCGAAGCACAAAGCTCACTTAC |
| 71 | OBD168-p567 | AGTCTCTGTTACCCAGGCTGGAGTTCGAAAGATGAG |
| 72 | OBD168-p1839 | GAAACATACAAAATTATCTCGAGGTTCATCCATGTTCTGG |
| 73 | OBD168-p751 | TGGGAGCTCTGATAGTGTTCTCTCGAAGGGAGC |
| 74 | OBD168-p1463 | ACGTTGGCCAGGCTGATCTCGACACATGTCTCATTGGATG |
| 75 | OBD168-p1031 | TGGCTCGACTAGCCACAGCTACTGGTCCCCA |
| 76 | OBD168-p1547 | TCTCTCTGTCTCTCAGATTCGAGTGAAGATCAAAGACTTG |
| 77 | OBD168-p1931 | CCACTTGAGCCTGGGAGGTCGATCCATGTACATTTGGAAA |
| 78 | OBD168-p1415 | TCCTTTTACTTCCGCCTCTCGAACACCACTGCCCTAGGGC |
| 79 | OBD168-p1627 | TGAATTTTCAACTTTGACTCGAGTTTGGGAATCACTGGGC |
| 80 | OBD168-p643 | TGCCTGGACTAGAGTGCAGTGGTTCGAGGTTTAC |
| 81 | OBD168-p1619 | TCTTCTGGTCCAGTCTCCTCGAGGTCAACAACCACTTCCC |
| 82 | OBD168-p687 | ACCATTCGAACTGTCTTCCTTTCCTGCCCACCAC |
| 83 | OBD168-p1159 | TGCTTGAGCCCAGGAGTTCGATTTCCTAGGCTC |
| 84 | OBD168-p563 | AGGAGTTCGAGCTCCTGACCTCAAGTGATCCC |
| 85 | OBD168-p755 | ACCTGCGCTTCCTGAGCATCGAAGAGAGAGATTG |
| 86 | OBD168-p1895 | ATGTTGGCCAGGCTGGTCTCGAGACCAGCCTGGCCAACAT |
| 87 | OBD168-p1455 | TTGCTTGAGGCCAGGATTTCGACCCCAAGTTCCCTCCATG |
| 88 | OBD168-p1987 | GAATAACATTCCATTGTATCGAGCACTCTCATGGCGACTT |
| 89 | OBD168-p843 | AAAGTCGAGTCTTGGCCATATAATGATGTAGCCACCAC |
| 90 | OBD168-p1131 | TAGTTCGAGGCGTGTATTATTTTGATCCAGTGCGCGTG |
| 91 | OBD168-p1823 | CTGGCTAATTTTTGTATTTCGATTAGGTAGATTTACCAAT |
| 92 | OBD168-p1335 | TTTAAACGTTCCATTTAGTCGACTAAAGGAAGCCACTGTT |
| 93 | OBD168-p983 | ATGCCGTGGGCCACATTCCGTCGAGATGAAGT |
| 94 | OBD168-p1471 | TCATCTGAGGTCAGGAGTTCGAACTCCTGACCTCAAATGA |
| 95 | OBD168-p1775 | ACTGATAGCGATGAAGATTCGAAGGGAGCATAATACAGGA |
| 96 | OBD168-p1635 | ATATTAAATGTCATGAGATCGACCTCCCAAAGTCCCGTGA |
| 97 | OBD168-p1475 | ATTCTGTAACCACGGCCCTCGATGGAATATTCTAGAGCAC |
| 98 | OBD168-p1459 | TCCCCCGAGGTCAGGAGTTCGATTAAGAGGCTAATTGGAA |
| 99 | OBD168-p1183 | TGAAGTCGAAGATGTTCAGACTCCCAAGGGTCGG |
| 100 | OBD168-p811 | AGCCCAGGAGTTCGAGGGCAGTATCCTCCATA |

TABLE 4.b10

| | | | |
|---|---|---|---|
| 51 | ORF1_12_51666821_51670814_51862305_51869704_FR | OBD168-1073.1075 | OBD168-q1073.q1075.p1073/p1075 |
| 52 | ORF1_7_137931629_137933966_137981878_137983291_FR | OBD168-873.875 | OBD168-q873.q875.p873/p875 |
| 53 | ORF1_11_61042263_61044402_61243450_61246026_FF | OBD168-1769.1771 | OBD168-q1769.q1771.p1769/p1771 |
| 54 | ORF1_X_2888191_2891631_3046105_3049187_RF | OBD168-1709.1711 | OBD168-q1709.q1711.p1709/p1711 |
| 55 | ORF1_11_115616166_115618345_115711125_115715158_FF | OBD168-1785.1787 | OBD168-q1785.q1787.p1785/p1787 |
| 56 | ORF1_X_77646855_77652042_77915112_77916580_RF | OBD168-2113.2115 | OBD168-q2113.q2115.p2113/p2115 |
| 57 | ORF1_20_62658422_62663845_62934812_62935956_RF | OBD168-1581.1583 | OBD168-q1581.q1583.p1581/p1583 |
| 58 | ORF1_22_17921708_17928023_17999187_18001605_FF | OBD168-933.935 | OBD168-q933.q935.p933/p935 |
| 59 | ORF1_19_47777294_47778517_47957709_47959307_RF | OBD168-1501.1503 | OBD168-q1501.q1503.p1501/p1503 |
| 60 | ORF1_8_7882531_7883805_7954782_7966362_FF | OBD168-2077.2079 | OBD168-q2077.q2079.p2077/p2079 |
| 61 | ORF1_11_12091217_12094358_12153671_12160688_FR | OBD168-937.939 | OBD168-q937.q939.p937/p939 |
| 62 | ORF1_17_31222339_31224131_31324781_31330350_RR | OBD168-1025.1027 | OBD168-q1025.q1027.p1025/p1027 |
| 63 | ORF1_7_4620261_4621735_4724415_4726417_FR | OBD168-1237.1239 | OBD168-q1237.q1239.p1237/p1239 |
| 64 | ORF1_10_45820548_45823847_45898343_45903899_RF | OBD168-1345.1347 | OBD168-q1345.q1347.p1345/p1347 |
| 65 | ORF1_16_81474879_81476453_81607034_81611259_RR | OBD168-1277.1279 | OBD168-q1277.q1279.p1277/p1279 |
| 66 | ORF1_11_10776569_10779775_10900691_10903608_FF | OBD168-1757.1759 | OBD168-q1757.q1759.p1757/p1759 |
| 67 | ORF1_8_97253466_97261198_97345458_97357412_RF | OBD168-721.723 | OBD168-q721.q723.p721/p723 |
| 68 | ORF1_1_109681178_109683602_109741801_109745521_FR | OBD168-1041.1043 | OBD168-q1041.q1043.p1041/p1043 |
| 69 | ORF1_1_7774817_7777030_7966809_7968736_FF | OBD168-1301.1303 | OBD168-q1301.q1303.p1301/p1303 |
| 70 | ORF1_12_7858281_7862390_7932397_7933511_RF | OBD168-1797.1799 | OBD168-q1797.q1799.p1797/p1799 |
| 71 | ORF1_13_30408291_30410407_30585632_30589891_RR | OBD168-565.567 | OBD168-q565.q567.p565/p567 |
| 72 | ORF1_15_30288521_30290733_30544055_30547496_FR | OBD168-1837.1839 | OBD168-q1837.q1839.p1837/p1839 |
| 73 | ORF1_11_61152527_61154904_61243450_61246026_RF | OBD168-749.751 | OBD168-q749.q751.p749/p751 |
| 74 | ORF1_17_64322183_64329531_64384310_64385587_RF | OBD168-1461.1463 | OBD168-q1461.q1463.p1461/p1463 |
| 75 | ORF1_6_26459325_26460705_26493174_26499033_RR | OBD168-1029.1031 | OBD168-q1029.q1031.p1029/p1031 |
| 76 | ORF1_2_74503923_74507137_74519716_74521847_FF | OBD168-1545.1547 | OBD168-q1545.q1547.p1545/p1547 |
| 77 | ORF1_2_159230477_159238639_159250572_159255062_FF | OBD168-1929.1931 | OBD168-q1929.q1931.p1929/p1931 |
| 78 | ORF1_16_46899253_46904082_47060044_47061469_FF | OBD168-1413.1415 | OBD168-q1413.q1415.p1413/p1415 |
| 79 | ORF1_5_40609009_40613245_40692971_40695207_FR | OBD168-1625.1627 | OBD168-q1625.q1627.p1625/p1627 |
| 80 | ORF1_4_47908113_47912137_48013964_48015665_FF | OBD168-641.643 | OBD168-q641.q643.p641/p643 |
| 81 | ORF1_4_38161792_38166364_38180538_38182770_FR | OBD168-1617.1619 | OBD168-q1617.q1619.p1617/p1619 |
| 82 | ORF1_7_22482163_22488443_22587350_22592472_RR | OBD168-685.687 | OBD168-q685.q687.p685/p687 |
| 83 | ORF1_17_27477797_27480875_27629083_27630392_RR | OBD168-1157.1159 | OBD168-q1157.q1159.p1157/p1159 |
| 84 | ORF1_10_13707193_13712319_13854651_13857653_FR | OBD168-561.563 | OBD168-q561.q563.p561/p563 |
| 85 | ORF1_15_79006295_79007772_79123113_79127341_RF | OBD168-753.755 | OBD168-q753.q755.p753/p755 |
| 86 | ORF1_19_9777468_9779791_9828343_9833999_FF | OBD168-1893.1895 | OBD168-q1893.q1895.p1893/p1895 |
| 87 | ORF1_17_48576277_48578280_48760984_48766661_FF | OBD168-1453.1455 | OBD168-q1453.q1455.p1453/p1455 |
| 88 | ORF1_3_124942088_124944537_125145442_125149201_RR | OBD168-1985.1987 | OBD168-q1985.q1987.p1985/p1987 |
| 89 | ORF1_2_230319552_230322976_230433766_230436650_RR | OBD168-841.843 | OBD168-q841.q843.p841/p843 |
| 90 | ORF1_5_179455074_179456297_179632102_179634386_RF | OBD168-1129.1131 | OBD168-q1129.q1131.p1129/p1131 |
| 91 | ORF1_13_94395452_94397500_94478315_94481582_FR | OBD168-1821.1823 | OBD168-q1821.q1823.p1821/p1823 |
| 92 | ORF1_1_185482953_185486658_185569901_185571399_FF | OBD168-1333.1335 | OBD168-q1333.q1335.p1333/p1335 |
| 93 | ORF1_7_105801948_105809991_105902395_105905257_RF | OBD168-981.983 | OBD168-q981.q983.p981/p983 |
| 94 | ORF1_17_75411072_75413612_75649583_75651419_RF | OBD168-1469.1471 | OBD168-q1469.q1471.p1469/p1471 |
| 95 | ORF1_11_61224624_61227200_61378098_61379415_FF | OBD168-1773.1775 | OBD168-q1773.q1775.p1773/p1775 |
| 96 | ORF1_5_94419239_94427299_94505842_94514405_FR | OBD168-1633.1635 | OBD168-q1633.q1635.p1633/p1635 |
| 97 | ORF1_17_82434207_82440644_82511425_82512972_FR | OBD168-1473.1475 | OBD168-q1473.q1475.p1473/p1475 |
| 98 | ORF1_17_59893718_59895987_59916319_59918152_FR | OBD168-1457.1459 | OBD168-q1457.q1459.p1457/p1459 |
| 99 | ORF1_2_102051982_102057593_102268036_102274328_RR | OBD168-1181.1183 | OBD168-q1181.q1183.p1181/p1183 |
| 100 | ORF1_1_203288566_203291154_203473865_203477190_FR | OBD168-809.811 | OBD168-q809.q811.p809/p811 |

TABLE 5.a1

| | probe | gene.index | RP/Rsum | FC: (class1/class2) |
|---|---|---|---|---|
| 1 | ORF1_13_80048989_80053962_80108036_80110958_RR | 419 | 2825.713915 | 2.124095099 |
| 2 | ORF1_11_36263501_36265092_36503363_36509375_RR | 177 | 1213.237849 | 2.041459285 |
| 3 | ORF1_4_928981_931161_985198_989096_FF | 329 | 2294.052797 | 2.007502947 |
| 4 | ORF1_9_96135943_96140879_96269701_96274236_FF | 485 | 3195.533146 | 1.859266991 |
| 5 | ORF1_1_155206612_155209922_155288575_155290398_FR | 27 | 247.895163 | 1.852929501 |
| 6 | ORF1_1_185478961_185481250_185562847_185565090_FF | 698 | 4271.30051 | 1.749091064 |
| 7 | ORF1_16_30340361_30342037_30569930_30571793_FF | 907 | 5157.748755 | 1.725394607 |
| 8 | ORF1_6_169127198_169132046_169286051_169290790_RF | 159 | 1109.170033 | 1.659776024 |
| 9 | ORF1_18_75239929_75247051_75392542_75394957_FF | 911 | 5160.683472 | 1.654347844 |
| 10 | ORF1_15_31214670_31223381_31397997_31400727_RF | 629 | 3926.448123 | 1.639072304 |
| 11 | ORF1_12_12889292_12890821_12982042_12990253_FR | 831 | 4871.786943 | 1.638553157 |
| 12 | ORF1_12_124112544_124115693_124251083_124252846_FF | 60 | 504.1606399 | 1.635878194 |
| 13 | ORF1_20_35273707_35277578_35323324_35324547_RR | 773 | 4585.205636 | 1.610695569 |
| 14 | ORF1_6_17488412_17494115_17602256_17605423_FF | 73 | 577.5597894 | 1.607076672 |
| 15 | ORF1_6_169127198_169132046_169290790_169293970_RF | 194 | 1298.641194 | 1.605135763 |
| 16 | ORF1_11_118438429_118442280_118496275_118499072_RR | 276 | 1915.265338 | 1.602784411 |
| 17 | ORF1_12_427695_429020_611557_612617_FF | 1295 | 6696.150841 | 1.59866513 |
| 18 | ORF1_14_88868042_88879418_89003053_89008734_FF | 647 | 4056.574955 | 1.5955909 |
| 19 | ORF1_22_50531145_50533032_50776978_50783308_FR | 1970 | 8884.98958 | 1.553604813 |
| 20 | ORF1_X_2276455_2282906_2372831_2374536_FF | 511 | 3330.494838 | 1.542512184 |
| 21 | ORF1_6_17759689_17762431_17990489_17995237_RR | 1133 | 6061.245091 | 1.539957959 |
| 22 | ORF1_12_117309542_117310883_117405729_117409691_FF | 102 | 799.5396006 | 1.539917198 |
| 23 | ORF1_1_21177377_21179201_21360997_21362872_RF | 1984 | 8927.194783 | 1.527758749 |
| 24 | ORF1_8_7810112_7811114_7862332_7864865_FR | 172 | 1193.63032 | 1.525213622 |
| 25 | ORF1_9_37463569_37466721_37580276_37583007_RF | 1954 | 8845.11861 | 1.521474808 |
| 26 | ORF1_12_117381529_117383570_117405729_117409691_FF | 126 | 915.966393 | 1.510901505 |
| 27 | ORF1_3_13931883_13934230_14046920_14054882_RR | 1351 | 6875.273187 | 1.500353833 |
| 28 | ORF1_12_10092308_10096371_10157054_10158072_RF | 1461 | 7266.928609 | 1.49098816 |
| 29 | ORF1_7_143815670_143817160_143832880_143837398_FF | 2208 | 9517.746791 | 1.480804168 |
| 30 | ORF1_22_42535866_42536905_42603122_42604347_RR | 1781 | 8336.699623 | 1.478170729 |
| 31 | ORF1_2_230865528_230871371_230949383_230957363_RR | 588 | 3687.643417 | 1.477305491 |
| 32 | ORF1_11_12153671_12160688_12204447_12211177_FR | 175 | 1195.365584 | 1.475548393 |
| 33 | ORF1_16_23959562_23960563_24103092_24104963_RR | 754 | 4519.649801 | 1.472890202 |
| 34 | ORF1_2_60549072_60551678_60704331_60707715_RR | 203 | 1396.243105 | 1.459163877 |
| 35 | ORF1_20_45852988_45854096_46007260_46009918_FF | 227 | 1561.169981 | 1.448953404 |
| 36 | ORF1_16_75377735_75379311_75564971_75566881_FF | 232 | 1608.653115 | 1.448232463 |
| 37 | ORF1_11_12136334_12141132_12153671_12160688_FF | 263 | 1842.300047 | 1.445343477 |
| 38 | ORF1_3_129012383_129018436_129244687_129248922_FR | 1005 | 5541.80804 | 1.442388064 |
| 39 | ORF1_10_107634434_107636773_107740167_107742400_RR | 218 | 1498.254177 | 1.441567369 |
| 40 | ORF1_2_60549072_60551678_60708542_60711616_RF | 239 | 1675.8149 | 1.438133894 |
| 41 | ORF1_10_76940960_76942733_76997164_76999885_FF | 226 | 1559.386392 | 1.434805759 |
| 42 | ORF1_1_20115093_20117021_20174195_20175240_RR | 233 | 1621.68179 | 1.431876764 |
| 43 | ORF1_4_138242151_138246996_138326681_138334943_FF | 240 | 1689.640917 | 1.429825432 |
| 44 | ORF1_11_11944920_11948043_12153671_12160688_RF | 269 | 1868.062483 | 1.429441007 |
| 45 | ORF1_11_12153671_12160688_12359781_12366018_FR | 265 | 1843.29157 | 1.42750786 |
| 46 | ORF1_1_160215823_160220940_160398180_160400303_FR | 1459 | 7258.1481 | 1.427388698 |
| 47 | ORF1_6_36126333_36131280_36222214_36228907_RR | 2228 | 9560.939689 | 1.425190029 |
| 48 | ORF1_2_113266536_113267636_113504184_113510749_FF | 1601 | 7740.65394 | 1.422208337 |
| 49 | ORF1_11_11984245_11993733_12153671_12160688_RF | 292 | 2024.157273 | 1.421811212 |
| 50 | ORF1_1_67685932_67691400_67707953_67710845_RR | 291 | 2011.449609 | 1.420820804 |

TABLE 5.a2

| | | | | Probe sequence |
|---|---|---|---|---|
| | pfp | P.value | Type | 60 mer |
| 1 | 0.039118584 | 0.000116135 | SSc | CCTCCATTTCCACCCCCACGCTCAAACCTCGACTTAGAACTGTAATTGAATTTGTTATAA |
| 2 | 0.004577452 | 5.74067E-06 | SSc | ATGAAGATGAGCAACTTGTCTATCTTGTTCGACTCACTGCAACCCCCGCCTCTGGGTTCT |
| 3 | 0.024204606 | 5.64234E-05 | SSc | AGAGAATGGGACCCACTGGCACTGGTGATCGACTGCGCCCCGCTGCTGTTCCTAGACGCA |
| 4 | 0.051401642 | 0.000176638 | SSc | CGGGGCAAACATAAAGAAGGATTTAGTGTCGAACTCCTGACTTCAGGTGATCCACCCACC |
| 5 | 6.29435E-05 | 1.20415E-08 | SSc | AAAGATGAATAAAATTTGGTTCCTCCCTTCGACCCTGTGCGGACATTCCGCATACTGCCA |
| 6 | 0.09408723 | 0.00046532 | SSc | CCCCATACCTGCTCCAAGTCTTTCCATCTCGAAGTATCCTCCTTAGGAAAACCCACTGGA |
| 7 | 0.133670574 | 0.00085903 | SSc | CTCTGTATTTGGAAGCTGCTCTAAGGTTTCGAATCCCTAGCAGCCAGTCCCTGCTACTTC |
| 8 | 0.003661224 | 4.12467E-06 | SSc | AGATCTGGTCTATTTGGTTCTTTAAACCTCGAGGCTCATGCAGGCTAACTGAGGCACCTC |
| 9 | 0.133327303 | 0.000860603 | SSc | ATATATGATTTCTGAATAATCTTTTTGCTCGAGAGGGTTTGGCCCCCAGCGCACCTCTGC |
| 10 | 0.07907398 | 0.000352411 | SSc | GTGGAAGGTGGAGGAGGGTGGAGGAGGGTCGAGCTCCACTCAGCTGGGTTCAGACAAGCT |
| 11 | 0.121371898 | 0.000714635 | SSc | GGCGCCCCCGTTCAATCAGCCTGCATTGTCGAACTCCTAAGCTCAGGTGATCCACCTGCC |
| 12 | 0.000485193 | 2.06268E-07 | SSc | ACATCTCCCTTCGGCCCCTGTGGCCGGCTCGATTTCAAGAAGTTGGTGAAAGCAGTGTTG |
| 13 | 0.107154223 | 0.000586886 | SSc | ATCACTCCCTGGGCAACCACCCCAGGGCTCGACAAGGTCAAATGCACAGTATTATGTCTA |
| 14 | 0.000676047 | 3.49675E-07 | SSc | ATCTCTTGTCGGAATTATTGCAGTAGTCTCGATTACTTTTTCGTCTTGGGCTGATAATCT |
| 15 | 0.005359081 | 7.36643E-06 | SSc | CTGGACTTGATAGTTTTAATGGAATATGTCGAGGCTCATGCAGGCTAACTGAGGCACCTC |
| 16 | 0.015278779 | 2.98788E-05 | SSc | CCCCATGCCATCCTCCCCCCTCTACAATTCGAGAAGACAGTCCAGAGCTGAACCCACCCC |
| 17 | 0.213997083 | 0.001963554 | SSc | TTTGCCTGAGTTTTTAAAGCATTTTTCTTCGAGCCCTACCAACCCAGACAGCTCCTTCCC |
| 18 | 0.085636774 | 0.000392582 | SSc | ACAAATTGCTCAACCAAAATTCATATCATCGACAGGCACCTTTCCAGGCTCTTCCAAGAG |
| 19 | 0.335479296 | 0.00468271 | SSc | CAGCCTGGGCAATACAGTGAGACCCATCTCGAAGTCTGAGGTAAAACACAGAACAGGATT |
| 20 | 0.056111863 | 0.000203161 | SSc | GAGAGGGAGGGAGGAGGGAGAAGGGAGGTCGAACTCCCGACCTCAGGTGATTTGCCTGCC |
| 21 | 0.178943249 | 0.001436516 | SSc | CACCCTTTACCATGTTTGCAGGAGATGATCGAACTTCTGACCTCAGGTGATCTGCCCACC |
| 22 | 0.00167783 | 1.21259E-06 | SSc | ATGTAGCCCAGGCTGGTTGTCTTTTTTTTCGATCCACCCATCCACCCATGCTCCCATCCA |
| 23 | 0.337922691 | 0.004750336 | SSc | CAGTGGGTCAACTTGGCACATGGAGGCCTCGACACTTCCTCAGACGCTCTGAGAGGTGAA |
| 24 | 0.004436615 | 5.40686E-06 | SSc | CTCCTCCTTTGTAATAAGGGCACTGAATTCGATCTCAGACTCCATCAGAGCACAGCAGCC |
| 25 | 0.333652548 | 0.004619386 | SSc | TCCTCCCTACTGCCTCCCAGGCTCAAAATCGATATAATTCATGCTGAGTTCTTTAAGTAC |
| 26 | 0.00226586 | 2.02287E-06 | SSc | TATGGTCTCAGCAAAGCGAAGGAGAAAGTCGATCCACCCATCCACCCATCCTCCCATCCA |
| 27 | 0.222707957 | 0.002131849 | SSc | CAGTCTTTTGTGGAGAAATACTTGTGACTCGATCCCTTACTCCTGAGGCAACTGTGACAA |
| 28 | 0.244545841 | 0.002531487 | SSc | ACAGATTAAAACTATGAGGATATACCATTCGAGACCAGCCTGAGCAACATAGTGAGATCC |
| 29 | 0.368303263 | 0.005761956 | SSc | AGTATGAGCCACTCTCATTTTTAGCCCCTCGAGCGACGGAGGCGACGCGGCGGCTACCGG |
| 30 | 0.305811136 | 0.003859069 | SSc | GGCATGCATATCGCTTGAGGGCAGGAGTTCGAGATCGGACACCTCCAACTCCCGGTCTGG |
| 31 | 0.068646104 | 0.000285995 | SSc | ACCCTCCTCCCATCCCCCACCTGGGGACTCGAACTCCTGAACTTGCGATCTGCCCGCGTT |
| 32 | 0.004383924 | 5.43584E-06 | SSc | TTTCTCTCTTGTGGCCCCTCTGGACTCCTCGACCATATCAGTGAGTGGAGTCTATGGTGA |
| 33 | 0.10481268 | 0.000559952 | SSc | GTTTGTATCTATTTCTGTGCAGAAGTCCTCGAACTCCTGACCTCAGGTCATCCTCTCACT |
| 34 | 0.006670746 | 9.5948E-06 | SSc | ATTATTTGCCCACTCCCTTCCACTTCAATCGAAGATTTTTCTATTTATCTCTGATGTAGT |
| 35 | 0.00893888 | 1.43772E-05 | SSc | TTATTTATTTTTTTGTAGAGACAAGGTCTCGACTCTCCACGCATCTCTGCCACCCGAGTG |
| 36 | 0.009743215 | 1.60161E-05 | SSc | AGCTGGTTCTTTGAACAGATCAATAAAATCGAATCTTCGCGGACTCCACGCATCTGTGTT |
| 37 | 0.013966084 | 2.60253E-05 | SSc | CCCTAAGTGATACTTGGGTCATCTTTCTTCGAGGAGTCCAGAGGGGCCACAAGAGAGAAA |
| 38 | 0.151829671 | 0.001081155 | SSc | AAAATTTCATAAAATTCAAATTTTAGTGTCGAACTCCTAACCTCAGGTGATCCGTCTGCC |
| 39 | 0.008022575 | 1.23918E-05 | SSc | TTCACTTGGACTACCCTCTCCGTCCACCTCGAATTCTAGCAGGTTTTATTGTAAAATTGA |
| 40 | 0.010955144 | 1.85516E-05 | SSc | CCAAAGAGCTGAGACAAAAGTACAATGTTCGATTGAAGTGGAAGGGAGTGGGCAAATAAT |
| 41 | 0.008941487 | 1.4318E-05 | SSc | TCTAATCTGTGGCCCTGGAAAATCATGATCGAGCCATATTAGAGGCATTTGGAAAACTTT |
| 42 | 0.00998706 | 1.64877E-05 | SSc | CATCATCACTGATGCTCGTGGCAGCCTGTCGAGCTCCAGGAGTGATATTTAAGGCCCCAC |
| 43 | 0.011235739 | 1.91064E-05 | SSc | ATATGAAACATTAACAAAACTATCCGTATCGACACTCACACCCACACACACTCAGAGTCA |
| 44 | 0.014346301 | 2.73437E-05 | SSc | TTTCTCTCTTGTGGCCCCTCTGGACTCCTCGATGGGAATACTTAGAATTATAGTTTGAAA |
| 45 | 0.013887258 | 2.60752E-05 | SSc | TTTCTCTCTTGTGGCCCCTCTGGACTCCTCGATAAGAGAAACACTAAAGAACTCTTGGTT |
| 46 | 0.243967232 | 0.002522041 | SSc | AGCAGAGGGAGCCAAGTAAAAGACCACATCGACTATGTCCTCAAGGACAGGAGCTGTACA |
| 47 | 0.369992569 | 0.005840815 | SSc | TGGGAATTGGTGACTGGTTTCTTAGAGATCGAGTGCCCTTCCTGGAAGGTGTGAACGGAG |
| 48 | 0.271112479 | 0.003075432 | SSc | TAGCTTACACCCTACTTTCCCCAGTGCATCGATGATTTCTATTAATTGTTATTATATTTA |
| 49 | 0.01756551 | 3.6342E-05 | SSc | TTTCTCTCTTGTGGCCCCTCTGGACTCCTCGAGTTATCAAAATATTTTGAGAGACAGTAT |
| 50 | 0.0172379 | 3.55421E-05 | SSc | CCCTCGTCCGGTACCACGCCCCTCCCTCTCGATGCACCCCAACATATAACACAAAACTAG |

TABLE 5.a3

| | Probe Location | | | | | 4 kb Sequence Location | | | | |
|---|---|---|---|---|---|---|---|---|---|---|
| | | Start1 | End1 | Start2 | End2 | | Start1 | End1 | Start2 | End2 |
| 1 | 13 | 80048991 | 80049020 | 80108038 | 80108067 | 13 | 80048991 | 80052990 | 80108038 | 80112037 |
| 2 | 11 | 36263503 | 36263532 | 36503365 | 36503394 | 11 | 36263503 | 36267502 | 36503365 | 36507364 |
| 3 | 4 | 931130 | 931159 | 989065 | 989094 | 4 | 927160 | 931159 | 985095 | 989094 |
| 4 | 9 | 96140848 | 96140877 | 96274205 | 96274234 | 9 | 96136878 | 96140877 | 96270235 | 96274234 |
| 5 | 1 | 155209891 | 155209920 | 155288577 | 155288606 | 1 | 155205921 | 155209920 | 155288577 | 155292576 |
| 6 | 1 | 185481219 | 185481248 | 185565059 | 185565088 | 1 | 185477249 | 185481248 | 185561089 | 185565088 |
| 7 | 16 | 30342006 | 30342035 | 30571762 | 30571791 | 16 | 30338036 | 30342035 | 30567792 | 30571791 |
| 8 | 6 | 169127200 | 169127229 | 169290759 | 169290788 | 6 | 169127200 | 169131199 | 169286789 | 169290788 |
| 9 | 18 | 75247020 | 75247049 | 75394926 | 75394955 | 18 | 75243050 | 75247049 | 75390956 | 75394955 |
| 10 | 15 | 31214672 | 31214701 | 31400696 | 31400725 | 15 | 31214672 | 31218671 | 31396726 | 31400725 |
| 11 | 12 | 12890790 | 12890819 | 12982044 | 12982073 | 12 | 12886820 | 12890819 | 12982044 | 12986043 |
| 12 | 12 | 124115662 | 124115691 | 124252815 | 124252844 | 12 | 124111692 | 124115691 | 124248845 | 124252844 |
| 13 | 20 | 35273709 | 35273738 | 35323326 | 35323355 | 20 | 35273709 | 35277708 | 35323326 | 35327325 |
| 14 | 6 | 17494084 | 17494113 | 17605392 | 17605421 | 6 | 17490114 | 17494113 | 17601422 | 17605421 |
| 15 | 6 | 169127200 | 169127229 | 169293939 | 169293968 | 6 | 169127200 | 169131199 | 169289969 | 169293968 |
| 16 | 11 | 118438431 | 118438460 | 118496277 | 118496306 | 11 | 118438431 | 118442430 | 118496277 | 118500276 |
| 17 | 12 | 428989 | 429018 | 612586 | 612615 | 12 | 425019 | 429018 | 608616 | 612615 |
| 18 | 14 | 88879387 | 88879416 | 89008703 | 89008732 | 14 | 88875417 | 88879416 | 89004733 | 89008732 |
| 19 | 22 | 50533001 | 50533030 | 50776980 | 50777009 | 22 | 50529031 | 50533030 | 50776980 | 50780979 |
| 20 | X | 2282875 | 2282904 | 2374505 | 2374534 | X | 2278905 | 2282904 | 2370535 | 2374534 |
| 21 | 6 | 17759691 | 17759720 | 17990491 | 17990520 | 6 | 17759691 | 17763690 | 17990491 | 17994490 |
| 22 | 12 | 117310852 | 117310881 | 117409660 | 117409689 | 12 | 117306882 | 117310881 | 117405690 | 117409689 |
| 23 | 1 | 21177379 | 21177408 | 21362841 | 21362870 | 1 | 21177379 | 21181378 | 21358871 | 21362870 |
| 24 | 8 | 7811083 | 7811112 | 7862334 | 7862363 | 8 | 7807113 | 7811112 | 7862334 | 7866333 |
| 25 | 9 | 37463571 | 37463600 | 37582976 | 37583005 | 9 | 37463571 | 37467570 | 37579006 | 37583005 |
| 26 | 12 | 117383539 | 117383568 | 117409660 | 117409689 | 12 | 117379569 | 117383568 | 117405690 | 117409689 |
| 27 | 3 | 13931885 | 13931914 | 14046922 | 14046951 | 3 | 13931885 | 13935884 | 14046922 | 14050921 |
| 28 | 12 | 10092310 | 10092339 | 10158041 | 10158070 | 12 | 10092310 | 10096309 | 10154071 | 10158070 |
| 29 | 7 | 143817129 | 143817158 | 143837367 | 143837396 | 7 | 143813159 | 143817158 | 143833397 | 143837396 |
| 30 | 22 | 42535868 | 42535897 | 42603124 | 42603153 | 22 | 42535868 | 42539867 | 42603124 | 42607123 |
| 31 | 2 | 230865530 | 230865559 | 230949385 | 230949414 | 2 | 230865530 | 230869529 | 230949385 | 230953384 |
| 32 | 11 | 12160657 | 12160686 | 12204449 | 12204478 | 11 | 12156687 | 12160686 | 12204449 | 12208448 |
| 33 | 16 | 23959564 | 23959593 | 24103094 | 24103123 | 16 | 23959564 | 23963563 | 24103094 | 24107093 |
| 34 | 2 | 60549074 | 60549103 | 60704333 | 60704362 | 2 | 60549074 | 60553073 | 60704333 | 60708332 |
| 35 | 20 | 45854065 | 45854094 | 46009887 | 46009916 | 20 | 45850095 | 45854094 | 46005917 | 46009916 |
| 36 | 16 | 75379280 | 75379309 | 75566850 | 75566879 | 16 | 75375310 | 75379309 | 75562880 | 75566879 |
| 37 | 11 | 12141101 | 12141130 | 12160657 | 12160686 | 11 | 12137131 | 12141130 | 12156687 | 12160686 |
| 38 | 3 | 129018405 | 129018434 | 129244689 | 129244718 | 3 | 129014435 | 129018434 | 129244689 | 129248688 |
| 39 | 10 | 107634436 | 107634465 | 107740169 | 107740198 | 10 | 107634436 | 107638435 | 107740169 | 107744168 |
| 40 | 2 | 60549074 | 60549103 | 60711585 | 60711614 | 2 | 60549074 | 60553073 | 60707615 | 60711614 |
| 41 | 10 | 76942702 | 76942731 | 76999854 | 76999883 | 10 | 76938732 | 76942731 | 76995884 | 76999883 |
| 42 | 1 | 20115095 | 20115124 | 20174197 | 20174226 | 1 | 20115095 | 20119094 | 20174197 | 20178196 |
| 43 | 4 | 138246965 | 138246994 | 138334912 | 138334941 | 4 | 138242995 | 138246994 | 138330942 | 138334941 |
| 44 | 11 | 11944922 | 11944951 | 12160657 | 12160686 | 11 | 11944922 | 11948921 | 12156687 | 12160686 |
| 45 | 11 | 12160657 | 12160686 | 12359783 | 12359812 | 11 | 12156687 | 12160686 | 12359783 | 12363782 |
| 46 | 1 | 160220909 | 160220938 | 160398182 | 160398211 | 1 | 160216939 | 160220938 | 160398182 | 160402181 |
| 47 | 6 | 36126335 | 36126364 | 36222216 | 36222245 | 6 | 36126335 | 36130334 | 36222216 | 36226215 |
| 48 | 2 | 113267605 | 113267634 | 113510718 | 113510747 | 2 | 113263635 | 113267634 | 113506748 | 113510747 |
| 49 | 11 | 11984247 | 11984276 | 12160657 | 12160686 | 11 | 11984247 | 11988246 | 12156687 | 12160686 |
| 50 | 1 | 67685934 | 67685963 | 67707955 | 67707984 | 1 | 67685934 | 67689933 | 67707955 | 67711954 |

TABLE 5.a4

| | probe | Primer ID |
|---|---|---|
| 1 | ORF1_13_80048989_80053962_80108036_80110958_RR | OBD168-469 |
| 2 | ORF1_11_36263501_36265092_36503363_36509375_RR | OBD168-1245 |
| 3 | ORF1_4_928981_931161_985198_989096_FF | OBD168-1613 |
| 4 | ORF1_9_96135943_96140879_96269701_96274236_FF | OBD168-1697 |
| 5 | ORF1_1_155206612_155209922_155288575_155290398_FR | OBD168-489 |
| 6 | ORF1_1_185478961_185481250_185562847_185565090_FF | OBD168-629 |
| 7 | ORF1_16_30340361_30342037_30569930_30571793_FF | OBD168-1853 |
| 8 | ORF1_6_169127198_169132046_169286051_169290790_RF | OBD168-2045 |
| 9 | ORF1_18_75239929_75247051_75392542_75394957_FF | OBD168-1485 |
| 10 | ORF1_15_31214670_31223381_31397997_31400727_RF | OBD168-1113 |
| 11 | ORF1_12_12889292_12890821_12982042_12990253_FR | OBD168-1057 |
| 12 | ORF1_12_124112544_124115693_124251083_124252846_FF | OBD168-605 |
| 13 | ORF1_20_35273707_35277578_35323324_35324547_RR | OBD168-1037 |
| 14 | ORF1_6_17488412_17494115_17602256_17605423_FF | OBD168-2029 |
| 15 | ORF1_6_169127198_169132046_169290790_169293970_RF | OBD168-2049 |
| 16 | ORF1_11_118438429_118442280_118496275_118499072_RR | OBD168-1133 |
| 17 | ORF1_12_427695_429020_611557_612617_FF | OBD168-781 |
| 18 | ORF1_14_88868042_88879418_89003053_89008734_FF | OBD168-845 |
| 19 | ORF1_22_50531145_50533032_50776978_50783308_FR | OBD168-1973 |
| 20 | ORF1_X_2276455_2282906_2372831_2374536_FF | OBD168-1705 |
| 21 | ORF1_6_17759689_17762431_17990489_17995237_RR | OBD168-1233 |
| 22 | ORF1_12_117309542_117310883_117405729_117409691_FF | OBD168-1809 |
| 23 | ORF1_1_21177377_21179201_21360997_21362872_RF | OBD168-1305 |
| 24 | ORF1_8_7810112_7811114_7862332_7864865_FR | OBD168-597 |
| 25 | ORF1_9_37463569_37466721_37580276_37583007_RF | OBD168-677 |
| 26 | ORF1_12_117381529_117383570_117405729_117409691_FF | OBD168-1209 |
| 27 | ORF1_3_13931883_13934230_14046920_14054882_RR | OBD168-1001 |
| 28 | ORF1_12_10092308_10096371_10157054_10158072_RF | OBD168-1377 |
| 29 | ORF1_7_143815670_143817160_143832880_143837398_FF | OBD168-881 |
| 30 | ORF1_22_42535866_42536905_42603122_42604347_RR | OBD168-1597 |
| 31 | ORF1_2_230865528_230871371_230949383_230957363_RR | OBD168-649 |
| 32 | ORF1_11_12153671_12160688_12204447_12211177_FR | OBD168-1201 |
| 33 | ORF1_16_23959562_23960563_24103092_24104963_RR | OBD168-1121 |
| 34 | ORF1_2_60549072_60551678_60704331_60707715_RR | OBD168-1065 |
| 35 | ORF1_20_45852988_45854096_46007260_46009918_FF | OBD168-917 |
| 36 | ORF1_16_75377735_75379311_75564971_75566881_FF | OBD168-553 |
| 37 | ORF1_11_12136334_12141132_12153671_12160688_FF | OBD168-993 |
| 38 | ORF1_3_129012383_129018436_129244687_129248922_FR | OBD168-1993 |
| 39 | ORF1_10_107634434_107636773_107740167_107742400_RR | OBD168-465 |
| 40 | ORF1_2_60549072_60551678_60708542_60711616_RF | OBD168-1257 |
| 41 | ORF1_10_76940960_76942733_76997164_76999885_FF | OBD168-725 |
| 42 | ORF1_1_20115093_20117021_20174195_20175240_RR | OBD168-585 |
| 43 | ORF1_4_138242151_138246996_138326681_138334943_FF | OBD168-1621 |
| 44 | ORF1_11_11944920_11948043_12153671_12160688_RF | OBD168-1153 |
| 45 | ORF1_11_12153671_12160688_12359781_12366018_FR | OBD168-941 |
| 46 | ORF1_1_160215823_160220940_160398180_160400303_FR | OBD168-1105 |
| 47 | ORF1_6_36126333_36131280_36222214_36228907_RR | OBD168-1665 |
| 48 | ORF1_2_113266536_113267636_113504184_113510749_FF | OBD168-1921 |
| 49 | ORF1_11_11984245_11993733_12153671_12160688_RF | OBD168-1101 |
| 50 | ORF1_1_67685932_67691400_67707953_67710845_RR | OBD168-729 |

TABLE 5.a5

| | Sequence | Primer ID | Sequence |
|---|---|---|---|
| 1 | GAGCAGCCAGTCAGTAGGAGAAACCA | OBD168-471 | TTTGCTCTTCCCATTCCACCTCAGAA |
| 2 | GTCGTTGTCTGTCTCCCCACTGG | OBD168-1247 | GTCTTGCCATTGTTTGCTTCCTTTCA |
| 3 | GAGGGCTTTTGTGTCGCTTCACTC | OBD168-1615 | GTCTGCACGGCATCGTGCACACTG |
| 4 | GGGCGCTGCAGAACAGAGCCCG | OBD168-1699 | ACAAAGAAGTCAAAATAATTGT |
| 5 | GACCAAACGACTCACAGCCAGGC | OBD168-491 | CTTGGCAGTGGAGACATACAGCG |
| 6 | GTCTGGTCTGAAGAGGCTGGAGG | OBD168-631 | CAAGCCAGTGCCAGAAGACAAGC |
| 7 | GATCTCAAAAAAGCACTATTCCAT | OBD168-1855 | CCCCGGGCCTCGGCGCCAGGAGCG |
| 8 | GCCACACAGCTCGTGTGGGATT | OBD168-2047 | TTCACTTAGCCTGCATGAGCCT |
| 9 | TCACAATAATGGAGTACTAAAGC | OBD168-1487 | CTTTGAGGAAGTCAACTACTGAG |
| 10 | TGGAGCAGAACAGGGAGGTCAGA | OBD168-1115 | ATAGTCGGCACTGCTGAGCCTGG |
| 11 | TGGAAGAGGAGTGGGCAGTGGAG | OBD168-1059 | GCACCCTGTCCTTTTCTGTCCTC |
| 12 | TGAAGCACAATCGGGACCGTGTC | OBD168-607 | CCCTCATTGACCCACCTTTGCCG |
| 13 | TTGGGAGCCTTCTGGGAGAGAGG | OBD168-1039 | CTGTGACCAGTGCCAACCCATTG |
| 14 | CCCCATCTCAGTTGATGGCGA | OBD168-2031 | TGGCTAATTTTTGTATTTTTA |
| 15 | GAGACGGGGTTTCACCATGTTGGC | OBD168-2051 | TCACTTAGGCTGCATGGGCCTAGA |
| 16 | ATCAAGAGCACTCCAGAGCAGCAACC | OBD168-1135 | ACTGTCTATTGTCTTCTATGCCTGGG |
| 17 | CGCTGGAGAAGGCAGATGGAGAA | OBD168-783 | GCAGCCCCAGAAAAGCAGAGGGA |
| 18 | GCGTAACTTCTGGCTTCTGTGCC | OBD168-847 | GAGAAGGGCACAACAGAGGGAGC |
| 19 | AAAAAAATCATGTATTGGGCCACAAA | OBD168-1975 | GGAATGGTCATGTTGCTACCTAGGGT |
| 20 | GAGAGAGGGAGGGAGGGAGAGGGAGA | OBD168-1707 | GAGGCCAGGCTCAGGGCTGTAATCCC |
| 21 | GGTAGGTTTCACTGTGGCGTGGT | OBD168-1235 | GGCAAACCTCGGTGTTCCTTGGC |
| 22 | CACTGCAGCCTTGAACTCCTG | OBD168-1811 | AGATGGGTGGATGGAGGGGTG |
| 23 | AAGAACTTAACAAAGACTGGCC | OBD168-1307 | ATTAGATATGGCGGCTCAGGTC |
| 24 | GCAGACAAGTCCAGAAAGCCCGA | OBD168-599 | TGGGTGAGGCTCTGCTTCCACAC |
| 25 | TTATGTGTCTCTTCCTAAGGGAGTCC | OBD168-679 | GACTCACAAGACTGTCCCCAAAGTGG |
| 26 | TTGGGATGGGAACTGGAGGCTGG | OBD168-1211 | TAGGTGAATGGAGAGGTGGAGGG |
| 27 | CAGAGGTGGTGCTGGAGGCAGAT | OBD168-1003 | GGAGACACATCCTGCTGCCTTGT |
| 28 | AGAATTGCTTGAACCCAGGAGG | OBD168-1379 | GCTCAAGCAATCCTCCCACCTC |
| 29 | GGTGGTGTTATCTGTCCTTGGGC | OBD168-883 | GAATCAGGCTCCCTTCCCTCCGA |
| 30 | TCCCGGCCCACTGCAACCTCC | OBD168-1599 | CTCCTCTGTCCCCAGTGTTAC |
| 31 | GTCCCACCGCCCTACTTTGGAAA | OBD168-651 | TTCCCCACAGAGTGCCTGAGCCA |
| 32 | CCACTGTCCCGCTCTTTCCACAC | OBD168-1203 | CCAGAACAAGGCACAAAGCAGGC |
| 33 | AGTGAGGAAGTGACACCCAAGATTGC | OBD168-1123 | CCCTCAAGGAAGTAGCAAAAGCCAGA |
| 34 | GCTCTCGTTTTGCTCGCTCCTTACCA | OBD168-1067 | TATGAAAATAAGCCCACGCACTGGC |
| 35 | GGGAAGGAAAGAAAACAGTGAGGGTT | OBD168-919 | ATGAGTGTCTGGAGGGAGTCCTTTGG |
| 36 | GCAGTGTGAGGGAAATGTGTAGTAAG | OBD168-555 | TCAGCCCCGTTTGTTTCTCCCACAGA |
| 37 | GTGGCAAGGGCTGGAGAAACAAC | OBD168-995 | CCACTGTCCCGCTCTTTCCACAC |
| 38 | TGTCTCCATAGGGAGCAGAAG | OBD168-1995 | CCCACCTCTGACTCGGATAGT |
| 39 | ACACCCCTTGATGTTACTTTGTCACA | OBD168-467 | CTTTCGTTTCCCTTCTCCAAGTCCTT |
| 40 | CAATCCCTTTGTGACTTCTGACCGA | OBD168-1259 | ATTTTCTATTTCTTCCTCCCCTCGTA |
| 41 | ATCTAAGAGGCTGGGATGAGGTTTA | OBD168-727 | AATCCTGTCCCTTGGCACTGCTCTG |
| 42 | GCTAAAATGAGACAGACAGAATGGGC | OBD168-587 | GAAGTTGTCCATCTGGGTGAGGTTGC |
| 43 | AAGCAAAACTGTAGAAAATTTGG | OBD168-1623 | TGCCTGAGGTTAATGATTACCCT |
| 44 | AACTTGCTGTCATTCCACCACTGTCC | OBD168-1155 | GTTGTAGTGAATGGTTTCTCTTCTTC |
| 45 | CGCTCTTTCCACACTCACATCCCTGT | OBD168-943 | TAAACACTTGCCGAAATGAGAGCGG |
| 46 | CCCAACCCTTGTCCCTGTGAAGC | OBD168-1107 | GCCAAGAGCCTGAGGGACAATAG |
| 47 | CATGATCTTCATACCTGGCATCTG | OBD168-1667 | AGTATGATTGGCCTGGGAGAGTGC |
| 48 | AGTGAGGAGCTAGTGGCCAGGAGGGG | OBD168-1923 | CTCCCACCCAGGATATTAGGAAAAAA |
| 49 | CGCTCTTTCCACACTCACATCCCTGT | OBD168-1103 | CTATCCAGTGTGTTCATAAGAAGCCC |
| 50 | CCCACCTTATCCATCCTACACGC | OBD168-731 | GCCACAGGTCCCCTAAGTGCTAC |

TABLE 5.a6

| | qPCR Primer ID | Sequence | qPCR Primer ID |
|---|---|---|---|
| 1 | OBD168-q469 | GAGCAGCCAGTCAGTAGGAGAAACCAAA | OBD168-q471 |
| 2 | OBD168-q1245 | CCCACCCACCCCTCGCCCTGCTCC | OBD168-q1247 |
| 3 | OBD168-q1613 | CTGTGTTTAGCCCATAAACTTCA | OBD168-q1615 |
| 4 | OBD168-q1697 | AGCGGATGTCTCCAGGAGGAGG | OBD168-q1699 |
| 5 | OBD168-q489 | GGCTGGACTGTCTCAGGGACCTACTC | OBD168-q491 |
| 6 | OBD168-q629 | TAGGTCTGGTCTGAAGAGGCTGGAGGAT | OBD168-q631 |
| 7 | OBD168-q1853 | TAAGAACTGGTTTAGCTCAGG | OBD168-q1855 |
| 8 | OBD168-q2045 | CCTCCATGATCTGGGTTGCTTTCAA | OBD168-q2047 |
| 9 | OBD168-q1485 | GAGACAATAACTGTCTTAAAA | OBD168-q1487 |
| 10 | OBD168-q1113 | GGCTGGAGCAGAACAGGGAGGTCAGAGC | OBD168-q1115 |
| 11 | OBD168-q1057 | CCGACACAGGCGGGCAGCCACTCC | OBD168-q1059 |
| 12 | OBD168-q605 | AATGGTGAAGCACAATCGGGACCGTGTC | OBD168-q607 |
| 13 | OBD168-q1037 | GTGCTGCCCAGACAGGACCCCTTGC | OBD168-q1039 |
| 14 | OBD168-q2029 | GTCAGAGACCAAGGAGTCATTCC | OBD168-q2031 |
| 15 | OBD168-q2049 | GGCCAGGCTGGTTTTGAACTCC | OBD168-q2051 |
| 16 | OBD168-q1133 | GGCAGAGCACGCAGGAAACGGCTGG | OBD168-q1135 |
| 17 | OBD168-q781 | GTATCCGCTGGAGAAGGCAGATGGAGAA | OBD168-q783 |
| 18 | OBD168-q845 | GGACTGCGTAACTTCTGGCTTCTGTGCC | OBD168-q847 |
| 19 | OBD168-q1973 | AAGTCCATGAAATATTCGCAAAA | OBD168-q1975 |
| 20 | OBD168-q1705 | AAGGAAGGAGAGAGGGAGGGAGG | OBD168-q1707 |
| 21 | OBD168-q1233 | CACCTGCCCCTCCACCTGCTGAC | OBD168-q1235 |
| 22 | OBD168-q1809 | TGCCTCAGTCGCCTGAGTAGCTA | OBD168-q1811 |
| 23 | OBD168-q1305 | CTTAACAAAGACTGGCCACCACCATG | OBD168-q1307 |
| 24 | OBD168-q597 | CAGACAAGTCCAGAAAGCCCGAAGTTGG | OBD168-q599 |
| 25 | OBD168-q677 | GGACTGAGGCTTTCTTGCTCTTCTGTGC | OBD168-q679 |
| 26 | OBD168-q1209 | GAGGTTGGGATGGGAACTGGAGGCTGGG | OBD168-q1211 |
| 27 | OBD168-q1001 | GCTTGGCAGAGGTGGTGCTGGAGGCAGA | OBD168-q1003 |
| 28 | OBD168-q1377 | GCCGGTAATCCCAGCTACTTGG | OBD168-q1379 |
| 29 | OBD168-q881 | CTCTGAGGTAGGCTTGGCTTCGGTGGTG | OBD168-q883 |
| 30 | OBD168-q1597 | TTTTTTTTTGAGAGAGAGAGTC | OBD168-q1599 |
| 31 | OBD168-q649 | CAGCCAATCAGGTCCCACCGCCC | OBD168-q651 |
| 32 | OBD168-q1201 | CGCTGGACATCCCCACCGCACCTCC | OBD168-q1203 |
| 33 | OBD168-q1121 | CCAGTGAGGAAGTGACACCCAAGATTGC | OBD168-q1123 |
| 34 | OBD168-q1065 | GCTCTCGTTTTGCTCGCTCCTTACCAAC | OBD168-q1067 |
| 35 | OBD168-q917 | GGGATGAGGAGTAAGAGACACGGGACAG | OBD168-q919 |
| 36 | OBD168-q553 | GCTAAAGCAGTGTGAGGGAAATGTGTAG | OBD168-q555 |
| 37 | OBD168-q993 | GGAGCCTACCTCTTCCTTCTCACTGACC | OBD168-q995 |
| 38 | OBD168-q1993 | AAACTACAGCCTGTGGGCCAGTCC | OBD168-q1995 |
| 39 | OBD168-q465 | GGTATGGCAAGGGAGGTGAGGGTTG | OBD168-q467 |
| 40 | OBD168-q1257 | ATCTGCCTCTGCTTTTCTCTTCCCCTA | OBD168-q1259 |
| 41 | OBD168-q725 | GTATTTGGCAGGGAGTAGGTGGAGGGTA | OBD168-q727 |
| 42 | OBD168-q585 | GCTGTGAAGAGGCAAGAATGGGTATGGG | OBD168-q587 |
| 43 | OBD168-q1621 | AATAGTTGAAAATTTGTGATATTC | OBD168-q1623 |
| 44 | OBD168-q1153 | CGCTGGACATCCCCACCGCACCTCC | OBD168-q1155 |
| 45 | OBD168-q941 | CCGCTCTTTCCACACTCACATCCCTG | OBD168-q943 |
| 46 | OBD168-q1105 | CCCCAACCCTTGTCCCTGTGAAGC | OBD168-q1107 |
| 47 | OBD168-q1665 | AAGTCTGCACATTTAACTGTCAACT | OBD168-q1667 |
| 48 | OBD168-q1921 | GAGCTAGTGGCCAGGAGGGGCTCATG | OBD168-q1923 |
| 49 | OBD168-q1101 | CGCTGGACATCCCCACCGCACCTCC | OBD168-q1103 |
| 50 | OBD168-q729 | GCTGACACTTCCTCCAGGGCATCCCC | OBD168-q731 |

TABLE 5.a7

| | Sequence | qPCR Probe | Sequence |
|---|---|---|---|
| 1 | CAAGTTTGCTCTTCCCATTCCACCTCAG | OBD168-p469 | ACGCTCAAACCTCGACTTAGAACTGTAATTGAATTT |
| 2 | GGCAAAACCCCGTCTCTACTAAACACAA | OBD168-p1245 | AGCAACTTGTCTATCTTGTTCGACTCACTGCAACCCC |
| 3 | TCAGCCGTGTCCCCGGGGCCCTC | OBD168-p1613 | ACCCACTGGCACTGGTGATCGACTGCGCCCCGCTGCTGTT |
| 4 | AAAACTGTCTAACAAAGAAGTC | OBD168-p1697 | ATAAAGAAGGATTTAGTGTCGAACTCCTGACTTCAGGTGA |
| 5 | CGGGTGAATGGATTATGATGGGACAAGC | OBD168-p489 | AATTTGGTTCCTCCCTTCGACCCTGTGCGGAC |
| 6 | GGTTCTGTATGGTGGATGGGCAAGGGC | OBD168-p629 | ACCTGCTCCAAGTCTTTCCATCTCGAAGTATCCTCC |
| 7 | GCCTCGGCGCCAGGAGCGGGG | OBD168-p1853 | GGAAGCTGCTCTAAGGTTTCGAATCCCTAGCAGCCAGTCC |
| 8 | CTCACTTAGGCTGCATGGGCCTAGA | OBD168-p2045 | TATTTGGTTCTTTAAACCTCGAGGCTCATGCAGGCTAACT |
| 9 | ACCTGAACTGGGGTGTGAGAG | OBD168-p1485 | TCTGAATAATCTTTTTGCTCGAGAGGGTTTGGCCCCCAGC |
| 10 | CCTTCTGTGCCCACGCCTGGTGC | OBD168-p1113 | AGGAGGGTCGAGCTCCACTCAGCTGGGTT |
| 11 | GGGACCAAGGGTTTTCAGTCTTAGCACC | OBD168-p1057 | TCAGCCTGCATTGTCGAACTCCTAAGCTCAGGTG |
| 12 | CGTATCCCCTGGTGGGCTCCTGAGGC | OBD168-p605 | TGTGGCCGGCTCGATTTCAAGAAGTTGGTGAAAGC |
| 13 | CTGTGACCAGTGCCAACCCATTGTAACC | OBD168-p1037 | AACCACCCCAGGGCTCGACAAGGTCAAATGCA |
| 14 | AGGTGCATACCACCACACCTGGC | OBD168-p2029 | GGAATTATTGCAGTAGTCTCGATTACTTTTTCGTCTTGGG |
| 15 | CTCACTTAGGCTGCATGGGCCT | OBD168-p2049 | TAGTTTTAATGGAATATGTCGAGGCTCATGCAGGCTAACT |
| 16 | CCCAGTCATCAAGTATGGACACAAGGAC | OBD168-p1133 | TCTACAATTCGAGAAGACAGTCCAGAGCTGAACCC |
| 17 | GCCAGGCAGCCCCAGAAAAGCAGAGGG | OBD168-p781 | TTTTCTTCGAGCCCTACCAACCCAGACAGCTC |
| 18 | CAGGACAAGACTGGGATTTGCCACCCC | OBD168-p845 | TCATATCATCGACAGGCACCTTTCCAGGCTCTTCC |
| 19 | CTACCTAGGGTTAGGGTTTCCTT | OBD168-p1973 | AATACAGTGAGACCCATCTCGAAGTCTGAGGTAAAACACA |
| 20 | ACTTTGGGAGGCCGTGGTGGGAA | OBD168-p1705 | GAGGAGGGAGAAGGGAGGTCGAACTCCCGACCTCAGGTGA |
| 21 | GGTGGCTCCTGGCAAACCTCGGTGTTCC | OBD168-p1233 | AGATGATCGAACTTCTGACCTCAGGTGATCTGCCC |
| 22 | GGAGGGGTGGAGGGGTGGATAGA | OBD168-p1809 | GGCTGGTTGTGTTTTTTTTCGATCCACCCATCCACCCATC |
| 23 | GATTAGATATGGCGGCTCAGGTCATG | OBD168-p1305 | ACTTGGCACATGGAGGCCTCGACACTTCCTCAGACGCTCT |
| 24 | CCCCTTGGTCCTTGGGTGAGGCTCTGC | OBD168-p597 | AGGGCACTGAATTCGATCTCAGACTCCATCAGAGC |
| 25 | GAGACTCACAAGACTGTCCCCAAAGTGG | OBD168-p677 | TCCTCCCTACTGCCTCCCAGGCTCAAAATCGATATA |
| 26 | GAGGGTGGACAGACAGGGAGGTGG | OBD168-p1209 | AGCGAAGGAGAAAGTCGATCCACCCATCCACC |
| 27 | GCCTCTCCCTTCCCACCTACTTGGC | OBD168-p1001 | TGTGACTCGATCCCTTACTCCTGAGGCAACTGTG |
| 28 | CCTTGACCTCCCTGGCTCAAGC | OBD168-p1377 | ACTATGAGGATATACCATTCGAGACCAGCCTGAGCAACAT |
| 29 | GGGTCTCTGCGGGTTCTTGGATGCGAG | OBD168-p881 | TTTTAGCCCCTCGAGCGACGGAGGCGAC |
| 30 | TCTGTCCCCAGTGTTACCACAG | OBD168-p1597 | TCGCTTGAGGGCAGGAGTTCGAGATCGGACACCTCCAACT |
| 31 | CCTTCCCCACAGAGTGCCTGAGCCACAC | OBD168-p651 | ATCGCAAGTTCAGGAGTTCGAGTCCCCAGGTG |
| 32 | GCCCAGAACAAGGCACAAAGCAGGC | OBD168-p1201 | TCTTGTGGCCCCTCTGGACTCCTCGACCATAT |
| 33 | GCCCTCAAGGAAGTAGCAAAAGCCAGAA | OBD168-p1121 | TCTGTGCAGAAGTCCTCGAACTCCTGACCTCAG |
| 34 | CAGTCCTAAAGGTATCCTCTCTGCCAGG | OBD168-p1065 | TGATTATTTGCCCACTCCCTTCCACTTCAATCGAAGAT |
| 35 | GCAGTGGGCTGATACCGTCTCTCCG | OBD168-p917 | AGACAAGGTCTCGACTCTCCACGCATCTCTGC |
| 36 | GCCGTCAGCCCCGTTTGTTTCTGGG | OBD168-p553 | TCAATAAAATCGAATCTTCGCGGACTCCACGCATCTGTG |
| 37 | CCCCACCGCACCTCCTCACGAATCCTGA | OBD168-p993 | ACTTGGGTCATCTTTCTTCGAGGAGTCCAGAGGG |
| 38 | TCGGATAGTGCTGGGATTACAGGC | OBD168-p1993 | AAAATTCAAATTTTAGTGTCGAACTCCTAACCTCAGGTGA |
| 39 | TTGTTTGGTTTCCCTTCTCCAAGTCCTT | OBD168-p465 | ACTTGGACTACCCTCTCCGTCCACCTCGAATTC |
| 40 | GCTCTCGTTTTGCTCGCTCCTTACCA | OBD168-p1257 | AGTACAATGTTCGATTGAAGTGGAAGGGAGTGGGC |
| 41 | CCTGTCCCTTGGCACTGCTCTGGAG | OBD168-p725 | TCATGATCGAGCCATATTAGAGGCATTTGGAAAACT |
| 42 | GAGAAGTTGTCCATCTGGGTGAGGTTGC | OBD168-p585 | AGCCTGTCGAGCTCCAGGAGTGATATTTAAGGCC |
| 43 | CCTGCTGCTTGCCTGAGGTTAATG | OBD168-p1621 | TTAACAAAACTATCCGTATCGACACTCACACCCACACACA |
| 44 | GCTATCCCTACTCCCCTATCTCCAAACC | OBD168-p1153 | TTGTGGCCCCTCTGGACTCCTCGATGGGAATA |
| 45 | TAAACACTTGCCGAAATGAGAGCGG | OBD168-p941 | TCTCTTGTGGCCCCTCTGGACTCCTCGATAAG |
| 46 | GCCAAGAGCCTGAGGGACAATAGAATGG | OBD168-p1105 | AGACCACATCGACTATGTCCTCAAGGACAGGAGC |
| 47 | ATGATTGGCCTGGGAGAGTGCCATC | OBD168-p1665 | TGACTGGTTTGTTAGAGATCGAGTGCCCTTCCTGGAAGGT |
| 48 | ATGACCGAAGGGGTGTATACCCACTG | OBD168-p1921 | CCTACTTTCCCCAGTGCATCGATGATTTCTATTAATTGTT |
| 49 | GGGCAATGGATTAGGGACACTGAAGCAG | OBD168-p1101 | TCTCTTGTGGCCCCTCTGGACTCCTCGAGTTA |
| 50 | GGTCCCCTAAGTGCTACAAGTGTTTGCG | OBD168-p729 | TACCACGCCCCTCCCTCTCGATGCACC |

TABLE 5.a8

| | qPCR Probe | Sequence |
|---|---|---|
| 1 | OBD168-p471 | TCAATTACAGTTCTAAGTCGAGGTTTGAGCGTGGG |
| 2 | OBD168-p1247 | TGCAGTGAGTCGAACAAGATAGACAAGTTGCTCATC |
| 3 | OBD168-p1615 | AACAGCAGCGGGGCGCAGTCGATCACCAGTGCCAGTGGGT |
| 4 | OBD168-p1699 | TCACCTGAAGTCAGGAGTTCGACACTAAATCCTTCTTTAT |
| 5 | OBD168-p491 | TGTCCGCACAGGGTCGAAGGGAGGAACCAAAT |
| 6 | OBD168-p631 | AGGAGGATACTTCGAGATGGAAAGACTTGGAGCAGG |
| 7 | OBD168-p1855 | GGACTGGCTGCTAGGGATTCGAAACCTTAGAGCAGCTTCC |
| 8 | OBD168-p2047 | AGTTAGCCTGCATGAGCCTCGAGGTTTAAAGAACCAAATA |
| 9 | OBD168-p1487 | GCTGGGGGCCAAACCCTCTCGAGCAAAAAGATTATTCAGA |
| 10 | OBD168-p1115 | AACCCAGCTGAGTGGAGCTCGACCCTCCT |
| 11 | OBD168-p1059 | TCACCTGAGCTTAGGAGTTCGACAATGCAGGCTG |
| 12 | OBD168-p607 | ACCAACTTCTTGAAATCGAGCCGGCCACAGGG |
| 13 | OBD168-p1039 | ACATAATACTGTGCATTTGACCTTGTCGAGCCCTGGG |
| 14 | OBD168-p2031 | CCCAAGACGAAAAAGTAATCGAGACTACTGCAATAATTCC |
| 15 | OBD168-p2051 | AGTTAGCCTGCATGAGCCTCGACATATTCCATTAAAACTA |
| 16 | OBD168-p1135 | TCAGCTCTGGACTGTCTTCTCGAATTGTAGAGGG |
| 17 | OBD168-p783 | AGCTGTCTGGGTTGGTAGGGCTCGAAGAAAA |
| 18 | OBD168-p847 | TGGAAGAGCCTGGAAAGGTGCCTGTCGATGATATG |
| 19 | OBD168-p1975 | TGTGTTTTACCTCAGACTTCGAGATGGGTCTCACTGTATT |
| 20 | OBD168-p1707 | TCACCTGAGGTCGGGAGTTCGACCTCCCTTCTCCCTCCTC |
| 21 | OBD168-p1235 | AGATCACCTGAGGTCAGAAGTTCGATCATCTCCTGC |
| 22 | OBD168-p1811 | GATGGGTGGATGGGTGGATCGAAAAAAAAGACAACCAGCC |
| 23 | OBD168-p1307 | AGAGCGTCTGAGGAAGTGTCGAGGCCTCCATGTGCCAAGT |
| 24 | OBD168-p599 | TGCTCTGATGGAGTCTGAGATCGAATTCAGTGCCC |
| 25 | OBD168-p679 | TATATCGATTTTGAGCCTGGGAGGCAGTAGGGAGG |
| 26 | OBD168-p1211 | TGGGTGGATGGGTGGATCGACTTTCTCCTTCG |
| 27 | OBD168-p1003 | ACAGTTGCCTCAGGAGTAAGGGATCGAGTCACAAG |
| 28 | OBD168-p1379 | ATGTTGCTCAGGCTGGTCTCGAATGGTATATCCTCATAGT |
| 29 | OBD168-p883 | GTCGCCTCCGTCGCTCGAGGGGCTAAAA |
| 30 | OBD168-p1599 | AGTTGGAGGTGTCCGATCTCGAACTCCTGCCCTCAAGCGA |
| 31 | OBD168-p649 | CACCTGGGGACTCGAACTCCTGAACTTGCGAT |
| 32 | OBD168-p1203 | TCCACTCACTGATATGGTCGAGGAGTCCAGAGG |
| 33 | OBD168-p1123 | ACCTGAGGTCAGGAGTTCGAGGACTTCTGCAC |
| 34 | OBD168-p1067 | ATCTTCGATTGAAGTGGAAGGGAGTGGGCAAATAATC |
| 35 | OBD168-p919 | TGGCAGAGATGCGTGGAGAGTCGAGACCTTGT |
| 36 | OBD168-p555 | ACACAGATGCGTGGAGTCCGCGAAGATTCGATTTTATTG |
| 37 | OBD168-p995 | TGGCCCCTCTGGACTCCTCGAAGAAAGATGAC |
| 38 | OBD168-p1995 | TCACCTGAGGTTAGGAGTTCGACACTAAAATTTGAATTTT |
| 39 | OBD168-p467 | AGAATTCGAGGTGGACGGAGAGGGTAGTCCAAG |
| 40 | OBD168-p1259 | TGCCCACTCCCTTCCACTTCAATCGAACATTGTAC |
| 41 | OBD168-p727 | TTTTCCAAATGCCTCTAATATGGCTCGATCATGA |
| 42 | OBD168-p587 | AATATCACTCCTGGAGCTCGACAGGCTGCCAC |
| 43 | OBD168-p1623 | TGTGTGTGGGTGTGAGTGTCGATACGGATAGTTTTGTTAA |
| 44 | OBD168-p1155 | TTCTAAGTATTCCCATCGAGGAGTCCAGAGGG |
| 45 | OBD168-p943 | AGTTCTTTAGTGTTTCTCTTATCGAGGAGTCCAGAGGG |
| 46 | OBD168-p1107 | AGCTCCTGTCCTTGAGGACATAGTCGATGTGGTC |
| 47 | OBD168-p1667 | ACCTTCCAGGAAGGGCACTCGATCTCTAAGAAACCAGTCA |
| 48 | OBD168-p1923 | AACAATTAATAGAAATCATCGATGCACTGGGGAAAGTAGG |
| 49 | OBD168-p1103 | AATATTTTGATAACTCGAGGAGTCCAGAGGG |
| 50 | OBD168-p731 | GGTGCATCGAGAGGGAGGGGCGTGGTA |

TABLE 5.a9

| | probe | PCR Marker Set | qPCR Marker Set |
|---|---|---|---|
| 1 | ORF1_13_80048989_80053962_80108036_80110958_RR | OBD168-469.471 | OBD168-q469.q471.p469/p471 |
| 2 | ORF1_11_36263501_36265092_36503363_36509375_RR | OBD168-1245.1247 | OBD168-q1245.q1247.p1245/p1247 |
| 3 | ORF1_4_928981_931161_985198_989096_FF | OBD168-1613.1615 | OBD168-q1613.q1615.p1613/p1615 |
| 4 | ORF1_9_96135943_96140879_96269701_96274236_FF | OBD168-1697.1699 | OBD168-q1697.q1699.p1697/p1699 |
| 5 | ORF1_1_155206612_155209922_155288575_155290398_FR | OBD168-489.491 | OBD168-q489.q491.p489/p491 |
| 6 | ORF1_1_185478961_185481250_185562847_185565090_FF | OBD168-629.631 | OBD168-q629.q631.p629/p631 |
| 7 | ORF1_16_30340361_30342037_30569930_30571793_FF | OBD168-1853.1855 | OBD168-q1853.q1855.p1853/p1855 |
| 8 | ORF1_6_169127198_169132046_169286051_169290790_RF | OBD168-2045.2047 | OBD168-q2045.q2047.p2045/p2047 |
| 9 | ORF1_18_75239929_75247051_75392542_75394957_FF | OBD168-1485.1487 | OBD168-q1485.q1487.p1485/p1487 |
| 10 | ORF1_15_31214670_31223381_31397997_31400727_RF | OBD168-1113.1H5 | OBD168-q1113.q1115.p1113/p1115 |
| 11 | ORF1_12_12889292_12890821_12982042_12990253_FR | OBD168-1057.1059 | OBD168-q1057.q1059.p1057/p1059 |
| 12 | ORF1_12_124112544_124115693_124251083_124252846_FF | OBD168-605.607 | OBD168-q605.q607.p605/p607 |
| 13 | ORF1_20_35273707_35277578_35323324_35324547_RR | OBD168-1037.1039 | OBD168-q1037.q1039.p1037/p1039 |
| 14 | ORF1_6_17488412_17494115_17602256_17605423_FF | OBD168-2029.2031 | OBD168-q2029.q2031.p2029/p2031 |
| 15 | ORF1_6_169127198_169132046_169290790_169293970_RF | OBD168-2049.2051 | OBD168-q2049.q2051.p2049/p2051 |
| 16 | ORF1_11_118438429_118442280_118496275_118499072_RR | OBD168-1133.1135 | OBD168-q1133.q1135.p1133/p1135 |
| 17 | ORF1_12_427695_429020_611557_612617_FF | OBD168-781.783 | OBD168-q781.q783.p781/p783 |
| 18 | ORF1_14_88868042_88879418_89003053_89008734_FF | OBD168-845.847 | OBD168-q845.q847.p845/p847 |
| 19 | ORF1_22_50531145_50533032_50776978_50783308_FR | OBD168-1973.1975 | OBD168-q1973.q1975.p1973/p1975 |
| 20 | ORF1_X_2276455_2282906_2372831_2374536_FF | OBD168-1705.1707 | OBD168-q1705.q1707.p1705/p1707 |
| 21 | ORF1_6_17759689_17762431_17990489_17995237_RR | OBD168-1233.1235 | OBD168-q1233.q1235.p1233/p1235 |
| 22 | ORF1_12_117309542_117310883_117405729_117409691_FF | OBD168-1809.1811 | OBD168-q1809.q1811.p1809/p1811 |
| 23 | ORF1_1_21177377_21179201_21360997_21362872_RF | OBD168-1305.1307 | OBD168-q1305.q1307.p1305/p1307 |
| 24 | ORF1_8_7810112_7811114_7862332_7864865_FR | OBD168-597.599 | OBD168-q597.q599.p597/p599 |
| 25 | ORF1_9_37463569_37466721_37580276_37583007_RF | OBD168-677.679 | OBD168-q677.q679.p677/p679 |
| 26 | ORF1_12_117381529_117383570_117405729_117409691_FF | OBD168-1209.1211 | OBD168-q1209.q1211.p1209/p1211 |
| 27 | ORF1_3_13931883_13934230_14046920_14054882_RR | OBD168-1001.1003 | OBD168-q1001.q1003.p1001/p1003 |
| 28 | ORF1_12_10092308_10096371_10157054_10158072_RF | OBD168-1377.1379 | OBD168-q1377.q1379.p1377/p1379 |
| 29 | ORF1_7_143815670_143817160_143832880_143837398_FF | OBD168-881.883 | OBD168-q881.q883.p881/p883 |
| 30 | ORF1_22_42535866_42536905_42603122_42604347_RR | OBD168-1597.1599 | OBD168-q1597.q1599.p1597/p1599 |
| 31 | ORF1_2_230865528_230871371_230949383_230957363_RR | OBD168-649.651 | OBD168-q649.q651.p651/p649 |
| 32 | ORF1_11_12153671_12160688_12204447_12211177_FR | OBD168-1201.1203 | OBD168-q1201.q1203.p1201/p1203 |
| 33 | ORF1_16_23959562_23960563_24103092_24104963_RR | OBD168-1121.1123 | OBD168-q1121.q1123.p1121/p1123 |
| 34 | ORF1_2_60549072_60551678_60704331_60707715_RR | OBD168-1065.1067 | OBD168-q1065.q1067.p1065/p1067 |
| 35 | ORF1_20_45852988_45854096_46007260_46009918_FF | OBD168-917.919 | OBD168-q917.q919.p917/p919 |
| 36 | ORF1_16_75377735_75379311_75564971_75566881_FF | OBD168-553.555 | OBD168-q553.q555.p553/p555 |
| 37 | ORF1_11_12136334_12141132_12153671_12160688_FF | OBD168-993.995 | OBD168-q993.q995.p993/p995 |
| 38 | ORF1_3_129012383_129018436_129244687_129248922_FR | OBD168-1993.1995 | OBD168-q1993.q1995.p1993/p1995 |
| 39 | ORF1_10_107634434_107636773_107740167_107742400_RR | OBD168-465.467 | OBD168-q465.q467.p465/p467 |
| 40 | ORF1_2_60549072_60551678_60708542_60711616_RF | OBD168-1257.1259 | OBD168-q1257.q1259.p1257/p1259 |
| 41 | ORF1_10_76940960_76942733_76997164_76999885_FF | OBD168-725.727 | OBD168-q725.q727.p725/p727 |
| 42 | ORF1_1_20115093_20117021_20174195_20175240_RR | OBD168-585.587 | OBD168-q585.q587.p585/p587 |
| 43 | ORF1_4_138242151_138246996_138326681_138334943_FF | OBD168-1621.1623 | OBD168-q1621.q1623.p1621/p1623 |
| 44 | ORF1_11_11944920_11948043_12153671_12160688_RF | OBD168-1153.1155 | OBD168-q1153.q1155.pH53/pH55 |
| 45 | ORF1_11_12153671_12160688_12359781_12366018_FR | OBD168-941.943 | OBD168-q941.q943.p941/p943 |
| 46 | ORF1_1_160215823_160220940_160398180_160400303_FR | OBD168-1105.1107 | OBD168-q1105.q1107.p1105/p1107 |
| 47 | ORF1_6_36126333_36131280_36222214_36228907_RR | OBD168-1665.1667 | OBD168-q1665.q1667.p1665/p1667 |
| 48 | ORF1_2_113266536_113267636_113504184_113510749_FF | OBD168-1921.1923 | OBD168-q1921.q1923.p1921/p1923 |
| 49 | ORF1_11_11984245_11993733_12153671_12160688_RF | OBD168-1101.1103 | OBD168-q1101.q1103.p1101/p1103 |
| 50 | ORF1_1_67685932_67691400_67707953_67710845_RR | OBD168-729.731 | OBD168-q729.q731.p729/p731 |

TABLE 5.b1

| | | | | |
|---|---|---|---|---|
| 51 | ORF1_5_126558745_126564482_126687692_126691508_FR | 264 | 1843.189907 | 1.420399219 |
| 52 | ORF1_17_47945779_47949526_48160816_48163394_RR | 976 | 5412.489422 | 1.416395767 |
| 53 | ORF1_10_5798770_5800647_6041802_6045506_RF | 2312 | 9736.38043 | 1.413748387 |
| 54 | ORF1_12_57601753_57604119_57758134_57764996_FR | 257 | 1814.399786 | 1.411737622 |
| 55 | ORF1_14_106703042_106707057_106732214_106736829_RR | 318 | 2223.082668 | 1.409196975 |
| 56 | ORF1_11_57374233_57377459_57426633_57429019_FR | 371 | 2583.552924 | 1.402595132 |
| 57 | ORF1_8_62981921_62989688_63002922_63008042_FF | 1388 | 6990.773109 | 1.400141562 |
| 58 | ORF1_7_8143002_8149481_8335072_8341646_RR | 399 | 2740.467886 | 1.398079472 |
| 59 | ORF1_8_140596381_140598037_140783960_140789447_RR | 1434 | 7143.652507 | 1.395733401 |
| 60 | ORF1_20_45815058_45818479_46007260_46009918_FF | 305 | 2099.941321 | 1.3945552 |
| 61 | ORF1_11_12153671_12160688_12216760_12220209_FR | 320 | 2226.338597 | 1.392175625 |
| 62 | ORF1_7_102513081_102515918_102618627_102620296_RF | 336 | 2330.127212 | 1.391206245 |
| 63 | ORF1_8_72866427_72867681_72985368_72989505_FF | 1969 | 8881.585714 | 1.386833222 |
| 64 | ORF1_17_27528031_27530003_27672414_27678224_RR | 1789 | 8356.225976 | 1.386084205 |
| 65 | ORF1_16_21532877_21535918_21626611_21627744_RR | 340 | 2371.450535 | 1.383780942 |
| 66 | ORF1_17_46588843_46590052_46655874_46660167_FF | 391 | 2706.432558 | 1.38133938 |
| 67 | ORF1_2_112933066_112936593_113031116_113032593_RF | 361 | 2546.429792 | 1.380522992 |
| 68 | ORF1_11_68953615_68955511_69110685_69112785_FR | 346 | 2444.640804 | 1.377326917 |
| 69 | ORF1_1_2986093_2988416_3070581_3073621_RF | 353 | 2470.335537 | 1.37695303 |
| 70 | ORF1_2_233770451_233776693_233851260_233853895_RF | 393 | 2715.586333 | 1.376771276 |
| 71 | ORF1_11_123648319_123653877_123684415_123688115_FF | 1775 | 8318.413763 | 1.36946818 |
| 72 | ORF1_16_56302242_56305491_56354996_56357979_RF | 360 | 2542.016781 | 1.367449353 |
| 73 | ORF1_6_109242018_109247965_109423758_109426226_RR | 434 | 2895.083912 | 1.364761815 |
| 74 | ORF1_7_77473907_77477977_77652906_77658625_RR | 671 | 4142.290351 | 1.364479462 |
| 75 | ORF1_17_79647743_79650208_79779269_79782236_RF | 429 | 2880.594219 | 1.362329268 |
| 76 | ORF1_4_123369547_123373273_123505935_123508246_RR | 433 | 2894.809412 | 1.361853703 |
| 77 | ORF1_2_60549072_60551678_60585452_60591312_RR | 400 | 2741.589992 | 1.360642047 |
| 78 | ORF1_2_200711316_200714252_200958736_200960996_FR | 449 | 3000.314558 | 1.360550483 |
| 79 | ORF1_2_160492996_160495482_160608080_160611872_FF | 604 | 3774.294445 | 1.360353024 |
| 80 | ORF1_7_37779378_37787306_37904027_37907035_FR | 454 | 3030.39315 | 1.36003832 |
| 81 | ORF1_1_93609850_93612271_93677337_93680831_FF | 2270 | 9652.970143 | 1.360018148 |
| 82 | ORF1_13_41335196_41337563_41355649_41358912_RR | 446 | 2982.959423 | 1.359616571 |
| 83 | ORF1_1_192936077_192944754_193066855_193069148_FF | 406 | 2773.668969 | 1.35851117 |
| 84 | ORF1_19_52598706_52601696_52670081_52671998_RR | 410 | 2788.031445 | 1.358207302 |
| 85 | ORF1_12_4049880_4053103_4104763_4106745_RF | 930 | 5249.941945 | 1.356775693 |
| 86 | ORF1_5_74469407_74475515_74537627_74543794_RF | 432 | 2886.148978 | 1.35658488 |
| 87 | ORF1_7_127648061_127651700_127781428_127785589_RF | 1111 | 5955.410877 | 1.354984218 |
| 88 | ORF1_14_90485762_90489489_90636665_90642057_FR | 476 | 3140.736349 | 1.352015821 |
| 89 | ORF1_5_180083035_180085563_180303025_180304376_RF | 445 | 2971.913692 | 1.351046773 |
| 90 | ORF1_2_187296012_187300600_187333661_187349760_FF | 458 | 3062.446524 | 1.350778635 |
| 91 | ORF1_17_36104009_36106111_36150024_36152875_RR | 509 | 3316.432305 | 1.348582449 |
| 92 | ORF1_10_103675840_103680824_103756732_103768333_FR | 460 | 3074.121972 | 1.34764065 |
| 93 | ORF1_17_46172500_46175294_46370925_46372131_RF | 557 | 3570.187804 | 1.347271817 |
| 94 | ORF1_14_93886681_93889278_93972230_93975078_FR | 2931 | 11190.0887 | 1.346109013 |
| 95 | ORF1_5_179958098_179961598_180083035_180085563_FR | 520 | 3412.75877 | 1.345171283 |
| 96 | ORF1_6_10510556_10512653_10613542_10617081_RR | 861 | 4986.41428 | 1.344654345 |
| 97 | ORF1_3_123064243_123067734_123161904_123162916_RF | 2877 | 11062.72759 | 1.3445561 |
| 98 | ORF1_5_95749524_95751329_95836578_95843620_RF | 488 | 3218.60268 | 1.343903594 |
| 99 | ORF1_1_192936077_192944754_193111803_193113272_FF | 475 | 3134.512644 | 1.343621216 |
| 100 | ORF1_16_11305532_11309621_11461548_11463016_RF | 2558 | 10326.79147 | 1.343510402 |

TABLE 5.b2

| | | | | |
|---|---|---|---|---|
| 51 | 0.013937125 | 2.60701E-05 | SSc | CATACATTTATTACTGATCTTTTGATCATCGACCTCCTAGCCCTGTCTCATCCAAGAGGG |
| 52 | 0.144987137 | 0.001002639 | SSc | TACCAGCACAGGCCCCGTTCCTCTCTGTTCGATTACCCCTGTAATGCCAGTGTGGTAATG |
| 53 | 0.376527839 | 0.006168083 | SSc | TATGTTCTAATCCTGGGAGTCTTCTCGGTCGAGACCAGCATGGCCAACAAGAAGAAATCC |
| 54 | 0.013535854 | 2.46481E-05 | SSc | GAACCATACACTTGACACCACCACCACCTCGAGAAGGTCCAGATTGCTGCCATAGTTCTG |
| 55 | 0.022433273 | 5.05458E-05 | SSc | TGACACAAAGATATTCTGCTTTGCCAGCTCGAAGGAATATTCTAGCCTTTGATTCCTTTC |
| 56 | 0.032448855 | 8.52979E-05 | SSc | CGCTACTCTCATTGGTCATACCTGTTGTTCGAATTCTGAATACCGTCCCCTAAAATAATG |
| 57 | 0.228289907 | 0.00224513 | SSc | CTGCACTCCAACAGAGGGAGACTCTGTCTCGATGCCCTGCCTGTGCCTGAGGTGATTAGG |
| 58 | 0.03697672 | 0.000104536 | SSc | AGGCTTATACTTTTATTGTATTTCTAAATCGAAGCAGGGAAGGGAGAAAAGGCCTTCTGA |
| 59 | 0.236300225 | 0.002400925 | SSc | GGGTTTCACCATGTTGGTCAGGCTGGTCTCGAGATCAGCCTGGCCAACGTTGTGAAACCC |
| 60 | 0.019145107 | 4.13736E-05 | SSc | GTAAGACCTTATCTCTAAAAGAAATGTTTCGACTCTCCACGCATCTCTGCCACCCGAGTG |
| 61 | 0.022407716 | 5.08057E-05 | SSc | TTTCTCTCTTGTGGCCCCTCTGGACTCCTCGATCTGCATCTTGACTCTGCCACATACCTC |
| 62 | 0.025028005 | 5.95842E-05 | SSc | GAGCCAAAGCATTTCCCGCTCTTAAACCTCGAGCCCCTTACTCTCCCTGGAGCCTTTGCT |
| 63 | 0.335260652 | 0.004677282 | SSc | ACATTTTTTGCTCCTGCTTGTTGAGGTTTCGAGACAAGCCTGGCCAACATGGTGAAACCC |
| 64 | 0.30662177 | 0.003886678 | SSc | AATGCATTAACTTACACAGAATACCTACTCGACCTAGGGAGACAAGTCCTGCCTCAGAAA |
| 65 | 0.026297675 | 6.33522E-05 | SSc | AAGGAAGGAAGCGTTGTTACCCAGGAGATCGAGTGGTTTGTCATGGTAAAGAAAAAATGC |
| 66 | 0.036144627 | 0.000100135 | SSc | AAACAACACTGGGGAATGAAAAGGTTGGTCGAAGATACTTTCAGATTATAGCCTTTGTTA |
| 67 | 0.0317184 | 8.11304E-05 | SSc | TACCTTTGACAAGACTCCCAGAGGCACTTCGAAGACCCACTTCCTACGTCAGACGTTTTC |
| 68 | 0.028725693 | 7.04226E-05 | SSc | ACCCAAGTCTTTATTAGCGAAGTCACGTTCGATCCATCCATCCATTCATCCATCCAAACA |
| 69 | 0.029197732 | 7.30279E-05 | SSc | CGGTGCAGCGGCGGCCGCGGGCTCAGAATCGATCCATCCATCCATCATCCACTTATCTAT |
| 70 | 0.036381223 | 0.000101306 | SSc | CGTGGCCATTTGCACCACCGGGGTGTCCTCGAAACCAACCTGACCAACATGAAGAAACCT |
| 71 | 0.304798022 | 0.003833326 | SSc | GTCAAGGACTGCGCCCTCTCAGATTCTTTCGACTTATTTTTTTAAACCTTTTTCTCTTGC |
| 72 | 0.031615997 | 8.06445E-05 | SSc | GGGCCCAGCCTGACCAGGAAGGCTGCCTTCGACACACCCCCCCACCACCCACCTCCTCTC |
| 73 | 0.041038607 | 0.000126197 | SSc | CCTGACTTCATACTTGTTGACTTCTAAGTCGAGGGAGCCAGAGGGGGAATCCAACCAAAG |
| 74 | 0.088474811 | 0.000420637 | SSc | CACCTGCTTGGGACATGAGGGCTTCCCCTCGATATCTTAACCTCGTGATCCGCCTGCCTC |
| 75 | 0.040809999 | 0.000124048 | SSc | CCGGGCCCACCTGCGGGTGCACCTTAAATCGATCCACCTGACCCCTGCCTCCTCCCCAAC |
| 76 | 0.04112004 | 0.000126156 | SSc | TATCAATTTGTCTATTTTATTCCATTTATCGACAGGAAGGAAGGAGGAAGGGGCGCTCTG |
| 77 | 0.036936253 | 0.000104683 | SSc | ATTATTTGCCCACTCCCTTCCACTTCAATCGATTCACACACCATTCCTCCTCAGCACACC |
| 78 | 0.044813002 | 0.000142566 | SSc | AAAGTAAAACATGTCCTTTAATGGACATTCGAACACTTTACACTCAGTTCCTCACTGAAC |
| 79 | 0.07220987 | 0.000309029 | SSc | GAAGAGTGACAGAAGGAAGAGAAGTAAATCGATCTTCAGCACCCTGTTCCCAGGAAGTGG |
| 80 | 0.045852249 | 0.000147497 | SSc | AATGAAGCATTTACTTATGAGAATATTCTCGATCTGACCTTGTGATCCACCCGCCTCAGC |
| 81 | 0.37373372 | 0.006011093 | SSc | CAGGCCTTCTCTCTGTGCTATGAAGACTTCGAGGAGGAGGTTGAGCGGACTCCGAAGCAT |
| 82 | 0.04423046 | 0.000139772 | SSc | TCTCTGATATCTGACAGTTTTTATATTTTCGAATACCTGACCTCGTGATCCACCCGCCTC |
| 83 | 0.03787522 | 0.000108955 | SSc | TCCCCCACCTCCCTTCCAACCTTCCCCTTCGATTCATGTCAGTGACTTGCCATACATATC |
| 84 | 0.038177056 | 0.000110905 | SSc | AGGTCTTACTATGTTGCCTAGGCTGATTTCGATCACCTGCTCCACCCTGACTCATTCCAA |
| 85 | 0.137999299 | 0.000909338 | SSc | ACCTTCCGAGCCACCATTTCCCCACTGCTCGACTCCCCAGTCTGACCTGGCCTCACCTGG |
| 86 | 0.040794748 | 0.000124869 | SSc | AAGCCTTTTTGCCAAACTACAGGCTTATTCGACCGCCCACCCCTCTCCCACTCCCCATCC |
| 87 | 0.172610563 | 0.001358772 | SSc | TTTCTGGGTGACTGTAATTGCTAGATGTTCGAACTCCTGACCTCCTGATCCCCCTGCTTT |
| 88 | 0.049389272 | 0.000166573 | SSc | ACAGCCACAATCCCTTCAATGATGAGGCTCGACCAATTTCAAATGTATATATAGTCTTTA |
| 89 | 0.043772158 | 0.000138014 | SSc | GACCCCTGGCTTGCAACAGGTGCCTGAATCGAAGATCCTCCCCACCCGCCCCCCTCCCAC |
| 90 | 0.047109718 | 0.000152877 | SSc | CCATCTTCTCCCTTGCCCTCTATTATTTTCGATCTGTGTAAACAAAAGGGACACAGAACC |
| 91 | 0.055533477 | 0.00020028 | SSc | GAAAAAAGGACTCTGCCTACACCTTGACTCGATAAATGGTAGTGTGAAGTTGGCACTAGG |
| 92 | 0.047516156 | 0.000154869 | SSc | GGAAAAAAATATTTTTTTCTTTCACACTTCGACCCCACCCCACCACCTCCAATCCATCAG |
| 93 | 0.065030297 | 0.000256647 | SSc | AAACAACACTGGGGAATGAAAAGGTTGGTCGATTCAGAGAAAATCACAGATTGTTAAAAT |
| 94 | 0.44902263 | 0.00932501 | SSc | CAGCTTACAAGGGTGAGTTGTGCGTATTTCGACCTCCTTCCTTGCAATGCTCTCTCCTTC |
| 95 | 0.0598673 | 0.000220576 | SSc | AGGTGACTATTAAACTACCCAATAATTCTCGAAGATCCTCCCCACCCGCCCCCCTCCCAC |
| 96 | 0.126289423 | 0.000770434 | SSc | TTTTATTCCTCACCTGACTGGGAGCAGCTCGAGTCCAGTATTTTTCCAATACTAGTTGTG |
| 97 | 0.442278174 | 0.009015725 | SSc | CACCCTGGCTGGAAGGGATGGTGTGTTCTCGAGGTTGCAGAGCAGAGCATGCCAAGGAAG |
| 98 | 0.052345849 | 0.000180995 | SSc | CTAAGGAAGCAGGAGGTCTTCTGAATCCTCGATGACTGTGAGAAACTTTTGTTGCATTCA |
| 99 | 0.049161058 | 0.000165455 | SSc | TCCCCCACCTCCCTTCCAACCTTCCCCTTCGAACTGCTGAGTAGGAAGCGCTCTAGCAGG |
| 100 | 0.405690362 | 0.007352931 | SSc | ACTAATCTCCACACGGCAACCAGACACGTCGAGACCATCCTGGCTAACACAGTGCAACCC |

TABLE 5.b3

| | | | | | | | | | | |
|---|---|---|---|---|---|---|---|---|---|---|
| 51 | 5 | 126564451 | 126564480 | 126687694 | 126687723 | 5 | 126560481 | 126564480 | 126687694 | 126691693 |
| 52 | 17 | 47945781 | 47945810 | 48160818 | 48160847 | 17 | 47945781 | 47949780 | 48160818 | 48164817 |
| 53 | 10 | 5798772 | 5798801 | 6045475 | 6045504 | 10 | 5798772 | 5802771 | 6041505 | 6045504 |
| 54 | 12 | 57604088 | 57604117 | 57758136 | 57758165 | 12 | 57600118 | 57604117 | 57758136 | 57762135 |
| 55 | 14 | 106703044 | 106703073 | 106732216 | 106732245 | 14 | 106703044 | 106707043 | 106732216 | 106736215 |
| 56 | 11 | 57377428 | 57377457 | 57426635 | 57426664 | 11 | 57373458 | 57377457 | 57426635 | 57430634 |
| 57 | 8 | 62989657 | 62989686 | 63008011 | 63008040 | 8 | 62985687 | 62989686 | 63004041 | 63008040 |
| 58 | 7 | 8143004 | 8143033 | 8335074 | 8335103 | 7 | 8143004 | 8147003 | 8335074 | 8339073 |
| 59 | 8 | 140596383 | 140596412 | 140783962 | 140783991 | 8 | 140596383 | 140600382 | 140783962 | 140787961 |
| 60 | 20 | 45818448 | 45818477 | 46009887 | 46009916 | 20 | 45814478 | 45818477 | 46005917 | 46009916 |
| 61 | 11 | 12160657 | 12160686 | 12216762 | 12216791 | 11 | 12156687 | 12160686 | 12216762 | 12220761 |
| 62 | 7 | 102513083 | 102513112 | 102620265 | 102620294 | 7 | 102513083 | 102517082 | 102616295 | 102620294 |
| 63 | 8 | 72867650 | 72867679 | 72989474 | 72989503 | 8 | 72863680 | 72867679 | 72985504 | 72989503 |
| 64 | 17 | 27528033 | 27528062 | 27672416 | 27672445 | 17 | 27528033 | 27532032 | 27672416 | 27676415 |
| 65 | 16 | 21532879 | 21532908 | 21626613 | 21626642 | 16 | 21532879 | 21536878 | 21626613 | 21630612 |
| 66 | 17 | 46590021 | 46590050 | 46660136 | 46660165 | 17 | 46586051 | 46590050 | 46656166 | 46660165 |
| 67 | 2 | 112933068 | 112933097 | 113032562 | 113032591 | 2 | 112933068 | 112937067 | 113028592 | 113032591 |
| 68 | 11 | 68955480 | 68955509 | 69110687 | 69110716 | 11 | 68951510 | 68955509 | 69110687 | 69114686 |
| 69 | 1 | 2986095 | 2986124 | 3073590 | 3073619 | 1 | 2986095 | 2990094 | 3069620 | 3073619 |
| 70 | 2 | 233770453 | 233770482 | 233853864 | 233853893 | 2 | 233770453 | 233774452 | 233849894 | 233853893 |
| 71 | 11 | 123653846 | 123653875 | 123688084 | 123688113 | 11 | 123649876 | 123653875 | 123684114 | 123688113 |
| 72 | 16 | 56302244 | 56302273 | 56357948 | 56357977 | 16 | 56302244 | 56306243 | 56353978 | 56357977 |
| 73 | 6 | 109242020 | 109242049 | 109423760 | 109423789 | 6 | 109242020 | 109246019 | 109423760 | 109427759 |
| 74 | 7 | 77473909 | 77473938 | 77652908 | 77652937 | 7 | 77473909 | 77477908 | 77652908 | 77656907 |
| 75 | 17 | 79647745 | 79647774 | 79782205 | 79782234 | 17 | 79647745 | 79651744 | 79778235 | 79782234 |
| 76 | 4 | 123369549 | 123369578 | 123505937 | 123505966 | 4 | 123369549 | 123373548 | 123505937 | 123509936 |
| 77 | 2 | 60549074 | 60549103 | 60585454 | 60585483 | 2 | 60549074 | 60553073 | 60585454 | 60589453 |
| 78 | 2 | 200714221 | 200714250 | 200958738 | 200958767 | 2 | 200710251 | 200714250 | 200958738 | 200962737 |
| 79 | 2 | 160495451 | 160495480 | 160611841 | 160611870 | 2 | 160491481 | 160495480 | 160607871 | 160611870 |
| 80 | 7 | 37787275 | 37787304 | 37904029 | 37904058 | 7 | 37783305 | 37787304 | 37904029 | 37908028 |
| 81 | 1 | 93612240 | 93612269 | 93680800 | 93680829 | 1 | 93608270 | 93612269 | 93676830 | 93680829 |
| 82 | 13 | 41335198 | 41335227 | 41355651 | 41355680 | 13 | 41335198 | 41339197 | 41355651 | 41359650 |
| 83 | 1 | 192944723 | 192944752 | 193069117 | 193069146 | 1 | 192940753 | 192944752 | 193065147 | 193069146 |
| 84 | 19 | 52598708 | 52598737 | 52670083 | 52670112 | 19 | 52598708 | 52602707 | 52670083 | 52674082 |
| 85 | 12 | 4049882 | 4049911 | 4106714 | 4106743 | 12 | 4049882 | 4053881 | 4102744 | 4106743 |
| 86 | 5 | 74469409 | 74469438 | 74543763 | 74543792 | 5 | 74469409 | 74473408 | 74539793 | 74543792 |
| 87 | 7 | 127648063 | 127648092 | 127785558 | 127785587 | 7 | 127648063 | 127652062 | 127781588 | 127785587 |
| 88 | 14 | 90489458 | 90489487 | 90636667 | 90636696 | 14 | 90485488 | 90489487 | 90636667 | 90640666 |
| 89 | 5 | 180083037 | 180083066 | 180304345 | 180304374 | 5 | 180083037 | 180087036 | 180300375 | 180304374 |
| 90 | 2 | 187300569 | 187300598 | 187349729 | 187349758 | 2 | 187296599 | 187300598 | 187345759 | 187349758 |
| 91 | 17 | 36104011 | 36104040 | 36150026 | 36150055 | 17 | 36104011 | 36108010 | 36150026 | 36154025 |
| 92 | 10 | 103680793 | 103680822 | 103756734 | 103756763 | 10 | 103676823 | 103680822 | 103756734 | 103760733 |
| 93 | 17 | 46172502 | 46172531 | 46372100 | 46372129 | 17 | 46172502 | 46176501 | 46368130 | 46372129 |
| 94 | 14 | 93889247 | 93889276 | 93972232 | 93972261 | 14 | 93885277 | 93889276 | 93972232 | 93976231 |
| 95 | 5 | 179961567 | 179961596 | 180083037 | 180083066 | 5 | 179957597 | 179961596 | 180083037 | 180087036 |
| 96 | 6 | 10510558 | 10510587 | 10613544 | 10613573 | 6 | 10510558 | 10514557 | 10613544 | 10617543 |
| 97 | 3 | 123064245 | 123064274 | 123162885 | 123162914 | 3 | 123064245 | 123068244 | 123158915 | 123162914 |
| 98 | 5 | 95749526 | 95749555 | 95843589 | 95843618 | 5 | 95749526 | 95753525 | 95839619 | 95843618 |
| 99 | 1 | 192944723 | 192944752 | 193113241 | 193113270 | 1 | 192940753 | 192944752 | 193109271 | 193113270 |
| 100 | 16 | 11305534 | 11305563 | 11462985 | 11463014 | 16 | 11305534 | 11309533 | 11459015 | 11463014 |

TABLE 5.b4

| | | |
|---|---|---|
| 51 | ORF1_5_126558745_126564482_126687692_126691508_FR | OBD168-1637 |
| 52 | ORF1_17_47945779_47949526_48160816_48163394_RR | OBD168-1033 |
| 53 | ORF1_10_5798770_5800647_6041802_6045506_RF | OBD168-1749 |
| 54 | ORF1_12_57601753_57604119_57758134_57764996_FR | OBD168-1021 |
| 55 | ORF1_14_106703042_106707057_106732214_106736829_RR | OBD168-501 |
| 56 | ORF1_11_57374233_57377459_57426633_57429019_FR | OBD168-1161 |
| 57 | ORF1_8_62981921_62989688_63002922_63008042_FF | OBD168-1249 |
| 58 | ORF1_7_8143002_8149481_8335072_8341646_RR | OBD168-653 |
| 59 | ORF1_8_140596381_140598037_140783960_140789447_RR | OBD168-1693 |
| 60 | ORF1_20_45815058_45818479_46007260_46009918_FF | OBD168-1957 |
| 61 | ORF1_11_12153671_12160688_12216760_12220209_FR | OBD168-1125 |
| 62 | ORF1_7_102513081_102515918_102618627_102620296_RF | OBD168-1005 |
| 63 | ORF1_8_72866427_72867681_72985368_72989505_FF | OBD168-2085 |
| 64 | ORF1_17_27528031_27530003_27672414_27678224_RR | OBD168-1429 |
| 65 | ORF1_16_21532877_21535918_21626611_21627744_RR | OBD168-657 |
| 66 | ORF1_17_46588843_46590052_46655874_46660167_FF | OBD168-769 |
| 67 | ORF1_2_112933066_112936593_113031116_113032593_RF | OBD168-1565 |
| 68 | ORF1_11_68953615_68955511_69110685_69112785_FR | OBD168-1365 |
| 69 | ORF1_1_2986093_2988416_3070581_3073621_RF | OBD168-1725 |
| 70 | ORF1_2_233770451_233776693_233851260_233853895_RF | OBD168-1941 |
| 71 | ORF1_11_123648319_123653877_123684415_123688115_FF | OBD168-961 |
| 72 | ORF1_16_56302242_56305491_56354996_56357979_RF | OBD168-1261 |
| 73 | ORF1_6_109242018_109247965_109423758_109426226_RR | OBD168-973 |
| 74 | ORF1_7_77473907_77477977_77652906_77658625_RR | OBD168-1293 |
| 75 | ORF1_17_79647743_79650208_79779269_79782236_RF | OBD168-545 |
| 76 | ORF1_4_123369547_123373273_123505935_123508246_RR | OBD168-2009 |
| 77 | ORF1_2_60549072_60551678_60585452_60591312_RR | OBD168-1533 |
| 78 | ORF1_2_200711316_200714252_200958736_200960996_FR | OBD168-1573 |
| 79 | ORF1_2_160492996_160495482_160608080_160611872_FF | OBD168-497 |
| 80 | ORF1_7_37779378_37787306_37904027_37907035_FR | OBD168-1681 |
| 81 | ORF1_1_93609850_93612271_93677337_93680831_FF | OBD168-733 |
| 82 | ORF1_13_41335196_41337563_41355649_41358912_RR | OBD168-1817 |
| 83 | ORF1_1_192936077_192944754_193066855_193069148_FF | OBD168-1337 |
| 84 | ORF1_19_52598706_52601696_52670081_52671998_RR | OBD168-1281 |
| 85 | ORF1_12_4049880_4053103_4104763_4106745_RF | OBD168-509 |
| 86 | ORF1_5_74469407_74475515_74537627_74543794_RF | OBD168-969 |
| 87 | ORF1_7_127648061_127651700_127781428_127785589_RF | OBD168-2073 |
| 88 | ORF1_14_90485762_90489489_90636665_90642057_FR | OBD168-1833 |
| 89 | ORF1_5_180083035_180085563_180303025_180304376_RF | OBD168-1165 |
| 90 | ORF1_2_187296012_187300600_187333661_187349760_FF | OBD168-601 |
| 91 | ORF1_17_36104009_36106111_36150024_36152875_RR | OBD168-825 |
| 92 | ORF1_10_103675840_103680824_103756732_103768333_FR | OBD168-1349 |
| 93 | ORF1_17_46172500_46175294_46370925_46372131_RF | OBD168-493 |
| 94 | ORF1_14_93886681_93889278_93972230_93975078_FR | OBD168-637 |
| 95 | ORF1_5_179958098_179961598_180083035_180085563_FR | OBD168-829 |
| 96 | ORF1_6_10510556_10512653_10613542_10617081_RR | OBD168-1649 |
| 97 | ORF1_3_123064243_123067734_123161904_123162916_RF | OBD168-1097 |
| 98 | ORF1_5_95749524_95751329_95836578_95843620_RF | OBD168-2021 |
| 99 | ORF1_1_192936077_192944754_193111803_193113272_FF | OBD168-1341 |
| 100 | ORF1_16_11305532_11309621_11461548_11463016_RF | OBD168-1409 |

TABLE 5.b5

| | | | |
|---|---|---|---|
| 51 | ACCATGTAGCCCAGGCTGGTCTTG | OBD168-1639 | AAATACCATAATGGATTCAAGTTA |
| 52 | GGGATGGCTCTGGGTGAATGGCA | OBD168-1035 | CCCACAGCACCTTTCTATGGTTCC |
| 53 | ACGAAATGATGGGGTGGTGGGGTG | OBD168-1751 | TATTTTTTTGAGACAGTCTTGCTC |
| 54 | TGATGTGGGAAGCAGAGCAGAGC | OBD168-1023 | CAACCAGGAAGAGCGAGCCCAGA |
| 55 | CACACCCTGACACAATGACGAATGAA | OBD168-503 | TGGATGGACCCTTCCTCTTCACCAAG |
| 56 | GGTGGGCATCTTGTCTCTCAGCC | OBD168-1163 | GCAGCGGGAGTTTTGTCTTCTG |
| 57 | GGGACACAGCCAAGCCAAATCAC | OBD168-1251 | GCCAAGGCACAGAAGGCACAGAAG |
| 58 | CTATTCTCTACTCCTTTTCCACACAC | OBD168-655 | CAGAGCCTGTGACCCTCAGACTTTTG |
| 59 | CAACCTCCGCCTCCAGGGTTCA | OBD168-1695 | CTCACTGCAACCTCCGCCTCCC |
| 60 | TGGTTCACATCAGCAATCTCAA | OBD168-1959 | GAGGTCCAGGCTGCAGTGGGCT |
| 61 | CGCTCTTTCCACACTCACATCCC | OBD168-1127 | GTTTGCCCACAGTCTGGAGGAGG |
| 62 | CCAAAGTGAGAAAATGTGACCTACGA | OBD168-1007 | ATGAAGTGGGCAGGCAGTGTGAGTC |
| 63 | TGTTGATAATAAACAATGGCTCTT | OBD168-2087 | GTGCAATGGTGTGATCTCGGCTCA |
| 64 | GCTAAGGAACAAGAATTGCTTG | OBD168-1431 | CCAAGGCTCCCACTGGGTTGCA |
| 65 | ATGAACTGGAAGGTCTGGGCGTC | OBD168-659 | GGACACAGGAACCCAGGACCTTC |
| 66 | CAGTGGAGAAATGGTATGGTGGAAAC | OBD168-771 | CCCACCTCCTTTACTACCCAATGAGC |
| 67 | CAAGAATATCACAGACACTCCAC | OBD168-1567 | AGCCTCACTTTCTTCAACGGGGG |
| 68 | ACAGTAGTATCCGGTGCTGTGGGGGC | OBD168-1367 | GTTTGGATGGATGGATGGATGAATGG |
| 69 | AATCAAACCCGTGTAACGAGTC | OBD168-1727 | AAAGATAGACGAATGAATGATT |
| 70 | GGTGAACAATGTTTACTTCTTAA | OBD168-1943 | CTGTCGCCCAGGCTGGAGTGGAG |
| 71 | GGCTACACAGAGAGATTCCCTCG | OBD168-963 | GCAGAAGAGGTGTCCTGGGCTGT |
| 72 | TTGAGAAATGAGGTGGAAGTGGG | OBD168-1263 | TAGGGCTGGCTGGGAAGCAGAGGCTA |
| 73 | GTGACCTGGGAGAAAAGAGGGTGAGA | OBD168-975 | GAGAGTGTAAGAAGGAAAATAGGTGG |
| 74 | CTTGGGAGGTTGAGGCAGAAGAA | OBD168-1295 | AAAATGCTGGGATTACAGGTGTGAGC |
| 75 | GGGACGGAAAGGAACAGGAAGCA | OBD168-547 | GTGGGAGACAGGTGTGAGTTGGG |
| 76 | GTCTTGTAGTAGTACAAGAACA | OBD168-2011 | AAACAAAGGGGTTCTGCACCAT |
| 77 | TCTGTCACTGAGACATTGAGA | OBD168-1535 | ACGTGCATGGAAAAGGGCTCC |
| 78 | TTGGCCTGTTACTGGGTATAG | OBD168-1575 | ACTGATTAGCTGGCAGCAGTT |
| 79 | ATAGGTGAGAACATTTTGGCTCTGCC | OBD168-499 | GGGTTTGAGGGTTTTCTGGAATCCCC |
| 80 | AAGTCTCACAGGAAGTCATGCCACAG | OBD168-1683 | TGTGGTTCCCAAGTCATGTCAGTGTA |
| 81 | CGAGGGCTACAGTTTCCAGTGAG | OBD168-735 | AACACGCCGCCTTGCCAGATGCT |
| 82 | CAAATAACTTGTCCATTAAAAACCTT | OBD168-1819 | TTCCAATATAAACTAACTACATGGTC |
| 83 | GTGATTTTTAGTTAGATGAGTAA | OBD168-1339 | CATTTCAGTAAACCTACATTTTG |
| 84 | AGGCTGGAGTGTGGTGGCAAGAT | OBD168-1283 | TGAGGCAGGGTGGAGCAGGTGAT |
| 85 | GGGACACTCACACTCCAGGTTGT | OBD168-511 | CCGCCTGAGTCCCTACATAGTGC |
| 86 | GCTGGCACCCCTGCTGAGAAGAA | OBD168-971 | GCAGCAGGGACCGCTATGAAATG |
| 87 | ATTAGTGGTGAGATGACATTC | OBD168-2075 | ATCGTATCTACTTCCATGACT |
| 88 | GAGGCTAGGGATGGGGGGACT | OBD168-1835 | ATAATTCTAAATATTTTGAAA |
| 89 | TGGGAGGGTGACACATCCTGAGG | OBD168-1167 | TACAGCACAACTGACCTCCCAGC |
| 90 | GCACAAGGGCATAATCAGAAAGCCAC | OBD168-603 | CTGAAACAGGGATTGTATCTCCAGAG |
| 91 | GCTGCCTTTTGTCCCATCCCCAG | OBD168-827 | GACCAAGGGAGGTTCAGAGAAGG |
| 92 | TGCAAGACTTTATTCTAACAAT | OBD168-1351 | TCCTGGAGAGGCAGGTGAGAGG |
| 93 | AATCTTTCTCCTCTCAGCGTGCTTGC | OBD168-495 | GCTGATGAAAACTGGATTCCCTGGC |
| 94 | TCACACACCCGATGATGGACAGC | OBD168-639 | TCCTACACCTCCGAGCACTCCAG |
| 95 | GGAGAAGGGTTGAACTAAGTAAGGCA | OBD168-831 | AATACAGCACAACTGACCTCCCAGCC |
| 96 | CTCTTCCTTTTTTTTTTTTAGGTGAT | OBD168-1651 | GAAAAAACACTGTATACCATCACACA |
| 97 | CTCCATTCCCTCCCCAATCTCCC | OBD168-1099 | GGAGCCACAGATGGTTCTCACTG |
| 98 | GGGCCTGGCCTAGAGTAGGTGCTTAG | OBD168-2023 | TGACAAATGAACCTTCACAATAAGCA |
| 99 | TTTGTTTTTGAAAAGTTTTTCTGGG | OBD168-1343 | CCTAGTTCAGCTAAAGACGGGGTTC |
| 100 | CATTCAGTCCATCAGGAACTTGAGTC | OBD168-1411 | CTGTGTCGCCCAGGTTAGAGTGCAGT |

TABLE 5.b6

| | | | |
|---|---|---|---|
| 51 | OBD168-q1637 | TGAACTCTTGAGCTCAGGCAA | OBD168-q1639 |
| 52 | OBD168-q1033 | CACCACCTGGAGGACTACAGCGGAGC | OBD168-q1035 |
| 53 | OBD168-q1749 | ATACCCCTACAAGCAGCTGCC | OBD168-q1751 |
| 54 | OBD168-q1021 | GGATTGGGCGGAGACTGCTTGATGTGGG | OBD168-q1023 |
| 55 | OBD168-q501 | GCACACCCTGACACAATGACGAATGAAT | OBD168-q503 |
| 56 | OBD168-q1161 | GGTGGGCATCTTGTCTCTCAGCCATCCC | OBD168-q1163 |
| 57 | OBD168-q1249 | TGGCAGAAGGCAAAGGAGGAGCAAAGT | OBD168-q1251 |
| 58 | OBD168-q653 | CCTCGTTTCTATTCTCTACTCCTTTTCC | OBD168-q655 |
| 59 | OBD168-q1693 | CTGGAGTGCAATGGTGCCATCTCAGC | OBD168-q1695 |
| 60 | OBD168-q1957 | AAAGAAAAATAAATAAATCTT | OBD168-q1959 |
| 61 | OBD168-q1125 | CCCCACCGCACCTCCTCACGAATCCTGA | OBD168-q1127 |
| 62 | OBD168-q1005 | CTTCCAAAGTGAGAAAATGTGACCTACG | OBD168-q1007 |
| 63 | OBD168-q2085 | GTGTGTGATCTATAGACCTATGTA | OBD168-q2087 |
| 64 | OBD168-q1429 | CGCAGTGAGCCGTGATGGCACCAC | OBD168-q1431 |
| 65 | OBD168-q657 | GCTCACCCTGGCTGCCCTCTATTCC | OBD168-q659 |
| 66 | OBD168-q769 | GAATCTTTCTCCTCTCAGCGTGCTTGC | OBD168-q771 |
| 67 | OBD168-q1565 | ACAATGAGAACCAGTTGTTCACAA | OBD168-q1567 |
| 68 | OBD168-q1365 | CTGGGAACAGTAGTATCCGGTGCT | OBD168-q1367 |
| 69 | OBD168-q1725 | GCGGGAGGACCAGGAGCAGCTC | OBD168-q1727 |
| 70 | OBD168-q1941 | ATGTTTACTTCTTAAGAGAGCAG | OBD168-q1943 |
| 71 | OBD168-q961 | GCTGGCGGCTTCCAAGGCTACACAGAG | OBD168-q963 |
| 72 | OBD168-q1261 | TTTTGAGAAATGAGGTGGAAGTGGG | OBD168-q1263 |
| 73 | OBD168-q973 | GCTGGCTGCCCACTTTGTGACCTGG | OBD168-q975 |
| 74 | OBD168-q1293 | AGGGAGGCTGAGGCGGGTGGGAT | OBD168-q1295 |
| 75 | OBD168-q545 | GCACGCCTCTCTGCTGGGTGGCA | OBD168-q547 |
| 76 | OBD168-q2009 | ATGGTACTTCTGTGTGGTTTTAAT | OBD168-q2011 |
| 77 | OBD168-q1533 | TTGAGACGTTCCAGGCTGTCTTGC | OBD168-q1535 |
| 78 | OBD168-q1573 | GATGCAGGGTCTGCCTGGTCAACG | OBD168-q1575 |
| 79 | OBD168-q497 | ATAGGTGAGAACATTTTGGCTCTGCC | OBD168-q499 |
| 80 | OBD168-q1681 | ATGTGAAACATTTGGGGCAATAC | OBD168-q1683 |
| 81 | OBD168-q733 | GGGACCGAGGGCTACAGTTTCCAGTGAG | OBD168-q735 |
| 82 | OBD168-q1817 | ATCACTGTAAAATGAAGGCAATACC | OBD168-q1819 |
| 83 | OBD168-q1337 | TTTCTGGGTACTGGTGATTTTTAG | OBD168-q1339 |
| 84 | OBD168-q1281 | CAGGCTGGAGTGTGGTGGCAAGAT | OBD168-q1283 |
| 85 | OBD168-q509 | AAGGGCAGGCAGGACCAGAGAACCAGCC | OBD168-q511 |
| 86 | OBD168-q969 | GGCTGCTGGCACCCCTGCTGAGA | OBD168-q971 |
| 87 | OBD168-q2073 | CTTGTGAATTCTGATGCATGCTCTT | OBD168-q2075 |
| 88 | OBD168-q1833 | GCCTGGTGATCTTCCACACCT | OBD168-q1835 |
| 89 | OBD168-q1165 | CGTGCTGGGAGGGTGACACATCCTGAGG | OBD168-q1167 |
| 90 | OBD168-q601 | GCACAAGGGCATAATCAGAAAGCCACAA | OBD168-q603 |
| 91 | OBD168-q825 | GGAAACCAAGGCACAGAGAGGGACAGG | OBD168-q827 |
| 92 | OBD168-q1349 | CAAAATACATTTTCAAGCTGGCTG | OBD168-q1351 |
| 93 | OBD168-q493 | GAATCTTTCTCCTCTCAGCGTGCTTGC | OBD168-q495 |
| 94 | OBD168-q637 | TGGACAGCGGGACCTCGTGGTGC | OBD168-q639 |
| 95 | OBD168-q829 | CCTCTACAACTGCTAATGGGAGAAGGGT | OBD168-q831 |
| 96 | OBD168-q1649 | TTTGCCTGACACCGAGTGGCCC | OBD168-q1651 |
| 97 | OBD168-q1097 | CGTCTCTCCATTCCCTCCCCAATCTCCC | OBD168-q1099 |
| 98 | OBD168-q2021 | AGCTGGCACTGCCAGTGCACAG | OBD168-q2023 |
| 99 | OBD168-q1341 | TGTCATTTGTTTTTGAAAAGT | OBD168-q1343 |
| 100 | OBD168-q1409 | CCTATGGAACGCGTCTAGAATC | OBD168-q1411 |

TABLE 5.b7

| | | | |
|---|---|---|---|
| 51 | TAGACTGGGAGATAAAATATG | OBD168-p1637 | TTACTGATCTTTTGATCATCGACCTCCTAGCCCTGTCTCA |
| 52 | CCACAGCACCTTTCTATGGTTCCAACAG | OBD168-p1033 | AGCACAGGCCCCGTTCCTCTCTGTTCGATTAC |
| 53 | TTGTCCAAGCTGATGTGTAGT | OBD168-p1749 | TCCTGGGAGTCTTCTCGGTCGAGACCAGCATGGCCAACAA |
| 54 | GCTGAGTGAGGACCAGATGAGCCAGGAG | OBD168-p1021 | ACACTTGACACCACCACCACCTCGAGAAGGTC |
| 55 | CTGACTGGATGGACCCTTCCTCTTCACC | OBD168-p501 | TGACACAAAGATATTCTGCTTTGCCAGCTCGAAGGA |
| 56 | AGAAGTTGATGCGAGGAAGGGCGGCGG | OBD168-p1161 | TGGTCATACCTGTTGTTCGAATTCTGAATACCGTCCCC |
| 57 | GCCAAGGCACAGAAGGCACAGAAGTGAC | OBD168-p1249 | AGAGGGAGACTCTGTCTCGATGCCCTGCCT |
| 58 | TCAGAGCCTGTGACCCTCAGACTTTTGA | OBD168-p653 | TGTATTTCTAAATCGAAGCAGGGAAGGGAGAAAAGGCC |
| 59 | ACTTTGTGATAGTTTTTTTCCCCCCA | OBD168-p1693 | ATGTTGGTCAGGCTGGTCTCGAGATCAGCCTGGCCAACGT |
| 60 | TGCAGTGGGCTGATACCGTCT | OBD168-p1957 | ATCTCTAAAAGAAATGTTTCGACTCTCCACGCATCTCTGC |
| 61 | GGGAATCTACAAACCCACCCTCTGCCG | OBD168-p1125 | TCTGGACTCCTCGATCTGCATCTTGACTCTGCC |
| 62 | ATGAAGTGGGCAGGCAGTGTGAGTCCA | OBD168-p1005 | TCCCGCTCTTAAACCTCGAGCCCCTTACTCTC |
| 63 | TTTTTGAGACAGGGTTTTGCTCTT | OBD168-p2085 | CTCCTGCTTGTTGAGGTTTCGAGACAAGCCTGGCCAACAT |
| 64 | AGCTGGGCTGCCAGCAGACATGGG | OBD168-p1429 | CTTACACAGAATACCTACTCGACCTAGGGAGACAAGTCCT |
| 65 | GCCCAACAGGACACAGGAACCCAGGACC | OBD168-p657 | AGGAAGCGTTGTTACCCAGGAGATCGAGTGGTTTG |
| 66 | CCGTCACTTCCTGTCTCTCCCACCTCC | OBD168-p769 | AATGAAAAGGTTGGTCGAAGATACTTTCAGATTATAGCCT |
| 67 | TGTGACCGTTGTCTTGATTCCTGG | OBD168-p1565 | AAGACTCCCAGAGGCACTTCGAAGACCCACTTCCTACGTC |
| 68 | GTTTGGATGGATGGATGGATGAAT | OBD168-p1365 | TTATTAGCGAAGTCACGTTCGATCCATCCATCCATTCATC |
| 69 | GAATGAATGATTGATGGATGAT | OBD168-p1725 | GCGGCCGCGGGCTCAGAATCGATCCATCCATCCATCATCC |
| 70 | TTTTTTTTTTTAAGTAAGACGGA | OBD168-p1941 | TGCACCACCGGGGTGTCCTCGAAACCAACCTGACCAACAT |
| 71 | GTCCAGTGGGTCTCACCTCAGAGTCAGC | OBD168-p961 | TCAAGGACTGCGCCCTCTCAGATTCTTTCGACTTA |
| 72 | GGGAAGGAGGTAGGGCTGGCTGGG | OBD168-p1261 | AGCCTGACCAGGAAGGCTGCCTTCGACACA |
| 73 | CCCTCCTGCCACCCTCCTACTCCTTACC | OBD168-p973 | ACTTGTTGACTTCTAAGTCGAGGGAGCCAGAGGG |
| 74 | CAAAATGCTGGGATTACAGGTGTGAGC | OBD168-p1293 | TGCTTGGGACATGAGGGCTTCCCCTCGATATC |
| 75 | TGAGATGGGCTCCGTGTCCCCTGCTGGA | OBD168-p545 | TGCACCTTAAATCGATCCACCTGACCCCTGCC |
| 76 | CACCTTAAGTAAAGGAAAAAGCGT | OBD168-p2009 | TCTATTTTATTCCATTTATCGACAGGAAGGAAGGAGGAAG |
| 77 | ACCCTCTGGGTTCTTTCTCCACGT | OBD168-p1533 | CACTCCCTTCCACTTCAATCGATTCACACACCATTCCTCC |
| 78 | GGCAGCAGTTAAAATCATCATTAG | OBD168-p1573 | ATGTCCTTTAATGGACATTCGAACACTTTACACTCAGTTC |
| 79 | GGGTTTGAGGGTTTTCTGGAATCCCCAC | OBD168-p497 | AGAGAAGTAAATCGATCTTCAGCACCCTGTTCCCAGG |
| 80 | CAATAATTTGTGAAAATGGACTT | OBD168-p1681 | TTACTTATGAGAATATTCTCGATCTGACCTTGTGATCCAC |
| 81 | ACGGAACACGCCGCCTTGCCAGATGCT | OBD168-p733 | TGAAGACTTCGAGGAGGAGGTTGAGCGGACTC |
| 82 | AACAGGTTCCAATATAAACTAACTA | OBD168-p1817 | CTGACAGTTTTTATATTTTCGAATACCTGACCTCGTGATC |
| 83 | ATTTCAGTAAACCTACATTTTGTT | OBD168-p1337 | CCCTTCCAACCTTCCCCTTCGATTCATGTCAGTGACTTGC |
| 84 | GGAATGAGGCAGGGTGGAGCAGGTG | OBD168-p1281 | AGGCTGATTTCGATCACCTGCTCCACCCTGAC |
| 85 | CCACCCGCACCGCACACCAGTTG | OBD168-p509 | ACCATTTCCCCACTGCTCGACTCCCCAGTCT |
| 86 | GTTCAAGGAGCAGTGAGGGCACCCAGGG | OBD168-p969 | TGCCAAACTACAGGCTTATTCGACCGCCCACC |
| 87 | ATCGTATCTACTTCCATGACTGCAT | OBD168-p2073 | ACTGTAATTGCTAGATGTTCGAACTCCTGACCTCCTGATC |
| 88 | TTTCTGTTATTTTCTTTTTGA | OBD168-p1833 | TCCCTTCAATGATGAGGCTCGACCAATTTCAAATGTATAT |
| 89 | CCCAGGCTCCCTTGTCCGTCTCC | OBD168-p1165 | TGCAACAGGTGCCTGAATCGAAGATCCTCCCC |
| 90 | GAGGAACTGAAAGATTTGGGAACAACCT | OBD168-p601 | TCCATCTTCTCCCTTGCCCTCTATTATTTTCGATCTG |
| 91 | GAGGTCCACAGAGAGGAGACACAAGGC | OBD168-p825 | AGGACTCTGCCTACACCTTGACTCGATAAATGGTAGTG |
| 92 | CCTGGAGAGGCAGGTGAGAGGGCA | OBD168-p1349 | ATTTTTTTCTTTCACACTTCGACCCCACCCCACCACCTCC |
| 93 | GCTGAGAGGAAGAAGCCAGTAGAGAAGA | OBD168-p493 | AATGAAAAGGTTGGTCGATTCAGAGAAAATCACAGATTG |
| 94 | GACAGGACAAGGACCCACAGGCACCAGC | OBD168-p637 | ACAAGGGTGAGTTGTGCGTATTTCGACCTCCTTCC |
| 95 | GCCCAGGCTCCCTTGTCCGTCTCC | OBD168-p829 | AATAATTCTCGAAGATCCTCCCCACCCGCCCC |
| 96 | TATATAAAGCAAACAAGTTTCA | OBD168-p1649 | CACCTGACTGGGAGCAGCTCGAGTCCAGTATTTTTCCAAT |
| 97 | GCACAGGACGGAGCCACAGATGGTTCTC | OBD168-p1097 | TGGAAGGGATGGTGTGTTCTCGAGGTTGCAGAG |
| 98 | AGCAAAACCAAACCATCTTTTT | OBD168-p2021 | AGGAGGTCTTCTGAATCCTCGATGACTGTGAGAAACTTTT |
| 99 | ACGGGGTTCCTTGTCCCACTG | OBD168-p1341 | CCCTTCCAACCTTCCCCTTCGAACTGCTGAGTAGGAAGCG |
| 100 | TTTTTTTGAGACGGAGTCTCGC | OBD168-p1409 | ACACGGCAACCAGACACGTCGAGACCATCCTGGCTAACAC |

TABLE 5.b8

| | | |
|---|---|---|
| 51 | OBD168-p1639 | TGAGACAGGGCTAGGAGGTCGATGATCAAAAGATCAGTAA |
| 52 | OBD168-p1035 | GTAATCGAACAGAGAGGAACGGGGCCTGTGCT |
| 53 | OBD168-p1751 | TTGTTGGCCATGCTGGTCTCGACCGAGAAGACTCCCAGGA |
| 54 | OBD168-p1023 | TGGACCTTCTCGAGGTGGTGGTGGTGTCAAGT |
| 55 | OBD168-p503 | TCCTTCGAGCTGGCAAAGCAGAATATCTTTGTGTCAG |
| 56 | OBD168-p1163 | AGAATTCGAACAACAGGTATGACCAATGAGAGTAGCGG |
| 57 | OBD168-p1251 | AGGCAGGGCATCGAGACAGAGTCTCCCTCT |
| 58 | OBD168-p655 | AGGCCTTTTCTCCCTTCCCTGCTTCGATTTAGA |
| 59 | OBD168-p1695 | ACGTTGGCCAGGCTGATCTCGAGACCAGCCTGACCAACAT |
| 60 | OBD168-p1959 | GCAGAGATGCGTGGAGAGTCGAAACATTTCTTTTAGAGAT |
| 61 | OBD168-p1127 | AGAGTCAAGATGCAGATCGAGGAGTCCAGAGGG |
| 62 | OBD168-p1007 | GAGAGTAAGGGGCTCGAGGTTTAAGAGCGGGA |
| 63 | OBD168-p2087 | ATGTTGGCCAGGCTTGTCTCGAAACCTCAACAAGCAGGAG |
| 64 | OBD168-p1431 | AGGACTTGTCTCCCTAGGTCGAGTAGGTATTCTGTGTAAG |
| 65 | OBD168-p659 | ACCACTCGATCTCCTGGGTAACAACGCTTCCTTC |
| 66 | OBD168-p771 | TCTGAAAGTATCTTCGACCAACCTTTTCATTCCCCAGTG |
| 67 | OBD168-p1567 | GACGTAGGAAGTGGGTCTTCGAAGTGCCTCTGGGAGTCTT |
| 68 | OBD168-p1367 | GATGAATGGATGGATGGATCGAACGTGACTTCGCTAATAA |
| 69 | OBD168-p1727 | GGATGATGGATGGATGGATCGATTCTGAGCCCGCGGCCGC |
| 70 | OBD168-p1943 | ATGTTGGTCAGGTTGGTTTCGAGGACACCCCGGTGGTGCA |
| 71 | OBD168-p963 | TAAGTCGAAAGAATCTGAGAGGGCGCAGTCCTTGAC |
| 72 | OBD168-p1263 | TGTGTCGAAGGCAGCCTTCCTGGTCAGGCT |
| 73 | OBD168-p975 | TCCCTCGACTTAGAAGTCAACAAGTATGAAGTCAGGC |
| 74 | OBD168-p1295 | GATATCGAGGGGAAGCCCTCATGTCCCAAGCA |
| 75 | OBD168-p547 | TCAGGTGGATCGATTTAAGGTGCAC |
| 76 | OBD168-p2011 | CTTCCTCCTTCCTTCCTGTCGATAAATGGAATAAAATAGA |
| 77 | OBD168-p1535 | GGAGGAATGGTGTGTGAATCGATTGAAGTGGAAGGGAGTG |
| 78 | OBD168-p1575 | GAACTGAGTGTAAAGTGTTCGAATGTCCATTAAAGGACAT |
| 79 | OBD168-p499 | TCCTGGGAACAGGGTGCTGAAGATCGATTTACTTCTC |
| 80 | OBD168-p1683 | GTGGATCACAAGGTCAGATCGAGAATATTCTCATAAGTAA |
| 81 | OBD168-p735 | TCCGCTCAACCTCCTCCTCGAAGTCTTCATAGC |
| 82 | OBD168-p1819 | GATCACGAGGTCAGGTATTCGAAAATATAAAAACTGTCAG |
| 83 | OBD168-p1339 | GCAAGTCACTGACATGAATCGAAGGGGAAGGTTGGAAGGG |
| 84 | OBD168-p1283 | AGGGTGGAGCAGGTGATCGAAATCAGCCTAGG |
| 85 | OBD168-p511 | AGACTGGGGAGTCGAGCAGTGGGGAAATGGT |
| 86 | OBD168-p971 | TGGGCGGTCGAATAAGCCTGTAGTTTGGCAAA |
| 87 | OBD168-p2075 | GATCAGGAGGTCAGGAGTTCGAACATCTAGCAATTACAGT |
| 88 | OBD168-p1835 | ATATACATTTGAAATTGGTCGAGCCTCATCATTGAAGGGA |
| 89 | OBD168-p1167 | ATCTTCGATTCAGGCACCTGTTGCAAGCCAGG |
| 90 | OBD168-p603 | ACAGATCGAAAATAATAGAGGGCAAGGGAGAAGATGG |
| 91 | OBD168-p827 | ACACTACCATTTATCGAGTCAAGGTGTAGGCAGAGTCC |
| 92 | OBD168-p1351 | GGAGGTGGTGGGGTGGGGTCGAAGTGTGAAAGAAAAAAAT |
| 93 | OBD168-p495 | TCTCTGAATCGACCAACCTTTTCATTCCCCAGTGTTG |
| 94 | OBD168-p639 | AGGAAGGAGGTCGAAATACGCACAACTCACCCTTG |
| 95 | OBD168-p831 | AGGATCTTCGAGAATTATTGGGTAGTTTAATAGTCACC |
| 96 | OBD168-p1651 | ATTGGAAAAATACTGGACTCGAGCTGCTCCCAGTCAGGTG |
| 97 | OBD168-p1099 | TCTGCAACCTCGAGAACACACCATCCCTTCCAG |
| 98 | OBD168-p2023 | AAAAGTTTCTCACAGTCATCGAGGATTCAGAAGACCTCCT |
| 99 | OBD168-p1343 | CGCTTCCTACTCAGCAGTTCGAAGGGGAAGGTTGGAAGGG |
| 100 | OBD168-p1411 | GTGTTAGCCAGGATGGTCTCGACGTGTCTGGTTGCCGTGT |

TABLE 5.b9

| | | | |
|---|---|---|---|
| 51 | ORF1_5_126558745_126564482_126687692_126691508_FR | OBD168-1637.1639 | OBD168-q1637.q1639.p1637/p1639 |
| 52 | ORF1_17_47945779_47949526_48160816_48163394_RR | OBD168-1033.1035 | OBD168-q1033.q1035.p1033/p1035 |
| 53 | ORF1_10_5798770_5800647_6041802_6045506_RF | OBD168-1749.1751 | OBD168-q1749.q1751.p1749/p1751 |
| 54 | ORF1_12_57601753_57604119_57758134_57764996_FR | OBD168-1021.1023 | OBD168-q1021.q1023.p1021/p1023 |
| 55 | ORF1_14_106703042_106707057_106732214_106736829_RR | OBD168-501.503 | OBD168-q501.q503.p501/p503 |
| 56 | ORF1_11_57374233_57377459_57426633_57429019_FR | OBD168-1161.1163 | OBD168-q1161.q1163.p1161/p1163 |
| 57 | ORF1_8_62981921_62989688_63002922_63008042_FF | OBD168-1249.1251 | OBD168-q1249.q1251.p1249/p1251 |
| 58 | ORF1_7_8143002_8149481_8335072_8341646_RR | OBD168-653.655 | OBD168-q653.q655.p653/p655 |
| 59 | ORF1_8_140596381_140598037_140783960_140789447_RR | OBD168-1693.1695 | OBD168-q1693.q1695.p1693/p1695 |
| 60 | ORF1_20_45815058_45818479_46007260_46009918_FF | OBD168-1957.1959 | OBD168-q1957.q1959.p1957/p1959 |
| 61 | ORF1_11_12153671_12160688_12216760_12220209_FR | OBD168-1125.1127 | OBD168-q1125.q1127.p1125/p1127 |
| 62 | ORF1_7_102513081_102515918_102618627_102620296_RF | OBD168-1005.1007 | OBD168-q1005.q1007.p1005/p1007 |
| 63 | ORF1_8_72866427_72867681_72985368_72989505_FF | OBD168-2085.2087 | OBD168-q2085.q2087.p2085/p2087 |
| 64 | ORF1_17_27528031_27530003_27672414_27678224_RR | OBD168-1429.1431 | OBD168-q1429.q1431.p1429/p1431 |
| 65 | ORF1_16_21532877_21535918_21626611_21627744_RR | OBD168-657.659 | OBD168-q657.q659.p657/p659 |
| 66 | ORF1_17_46588843_46590052_46655874_46660167_FF | OBD168-769.771 | OBD168-q769.q771.p769/p771 |
| 67 | ORF1_2_112933066_112936593_113031116_113032593_RF | OBD168-1565.1567 | OBD168-q1565.q1567.p1565/p1567 |
| 68 | ORF1_11_68953615_68955511_69110685_69112785_FR | OBD168-1365.1367 | OBD168-q1365.q1367.p1365/p1367 |
| 69 | ORF1_1_2986093_2988416_3070581_3073621_RF | OBD168-1725.1727 | OBD168-q1725.q1727.p1725/p1727 |
| 70 | ORF1_2_233770451_233776693_233851260_233853895_RF | OBD168-1941.1943 | OBD168-q1941.q1943.p1941/p1943 |
| 71 | ORF1_11_123648319_123653877_123684415_123688115_FF | OBD168-961.963 | OBD168-q961.q963.p961/p963 |
| 72 | ORF1_16_56302242_56305491_56354996_56357979_RF | OBD168-1261.1263 | OBD168-q1261.q1263.p1261/p1263 |
| 73 | ORF1_6_109242018_109247965_109423758_109426226_RR | OBD168-973.975 | OBD168-q973.q975.p973/p975 |
| 74 | ORF1_7_77473907_77477977_77652906_77658625_RR | OBD168-1293.1295 | OBD168-q1293.q1295.p1293/p1295 |
| 75 | ORF1_17_79647743_79650208_79779269_79782236_RF | OBD168-545.547 | OBD168-q545.q547.p545/p547 |
| 76 | ORF1_4_123369547_123373273_123505935_123508246_RR | OBD168-2009.2011 | OBD168-q2009.q2011.p2009/p2011 |
| 77 | ORF1_2_60549072_60551678_60585452_60591312_RR | OBD168-1533.1535 | OBD168-q1533.q1535.p1533/p1535 |
| 78 | ORF1_2_200711316_200714252_200958736_200960996_FR | OBD168-1573.1575 | OBD168-q1573.q1575.p1573/p1575 |
| 79 | ORF1_2_160492996_160495482_160608080_160611872_FF | OBD168-497.499 | OBD168-q497.q499.p497/p499 |
| 80 | ORF1_7_37779378_37787306_37904027_37907035_FR | OBD168-1681.1683 | OBD168-q1681.q1683.p1681/p1683 |
| 81 | ORF1_1_93609850_93612271_93677337_93680831_FF | OBD168-733.735 | OBD168-q733.q735.p733/p735 |
| 82 | ORF1_13_41335196_41337563_41355649_41358912_RR | OBD168-1817.1819 | OBD168-q1817.q1819.p1817/p1819 |
| 83 | ORF1_1_192936077_192944754_193066855_193069148_FF | OBD168-1337.1339 | OBD168-q1337.q1339.p1337/p1339 |
| 84 | ORF1_19_52598706_52601696_52670081_52671998_RR | OBD168-1281.1283 | OBD168-q1281.q1283.p1281/p1283 |
| 85 | ORF1_12_4049880_4053103_4104763_4106745_RF | OBD168-509.511 | OBD168-q509.q511.p509/p511 |
| 86 | ORF1_5_74469407_74475515_74537627_74543794_RF | OBD168-969.971 | OBD168-q969.q971.p969/p971 |
| 87 | ORF1_7_127648061_127651700_127781428_127785589_RF | OBD168-2073.2075 | OBD168-q2073.q2075.p2073/p2075 |
| 88 | ORF1_14_90485762_90489489_90636665_90642057_FR | OBD168-1833.1835 | OBD168-q1833.q1835.p1833/p1835 |
| 89 | ORF1_5_180083035_180085563_180303025_180304376_RF | OBD168-1165.1167 | OBD168-q1165.q1167.p1165/p1167 |
| 90 | ORF1_2_187296012_187300600_187333661_187349760_FF | OBD168-601.603 | OBD168-q601.q603.p601/p603 |
| 91 | ORF1_17_36104009_36106111_36150024_36152875_RR | OBD168-825.827 | OBD168-q825.q827.p825/p827 |
| 92 | ORF1_10_103675840_103680824_103756732_103768333_FR | OBD168-1349.1351 | OBD168-q1349.q1351.p1349/p1351 |
| 93 | ORF1_17_46172500_46175294_46370925_46372131_RF | OBD168-493.495 | OBD168-q493.q495.p493/p495 |
| 94 | ORF1_14_93886681_93889278_93972230_93975078_FR | OBD168-637.639 | OBD168-q637.q639.p637/p639 |
| 95 | ORF1_5_179958098_179961598_180083035_180085563_FR | OBD168-829.831 | OBD168-q829.q831.p829/p831 |
| 96 | ORF1_6_10510556_10512653_10613542_10617081_RR | OBD168-1649.1651 | OBD168-q1649.q1651.p1649/p1651 |
| 97 | ORF1_3_123064243_123067734_123161904_123162916_RF | OBD168-1097.1099 | OBD168-q1097.q1099.p1097/p1099 |
| 98 | ORF1_5_95749524_95751329_95836578_95843620_RF | OBD168-2021.2023 | OBD168-q2021.q2023.p2021/p2023 |
| 99 | ORF1_1_192936077_192944754_193111803_193113272_FF | OBD168-1341.1343 | OBD168-q1341.q1343.p1341/p1343 |
| 100 | ORF1_16_11305532_11309621_11461548_11463016_RF | OBD168-1409.1411 | OBD168-q1409.q1411.p1409/pl411 |

TABLE 6.a1

| | probe | gene.index | RP/Rsum |
|---|---|---|---|
| 1 | ORF1_19_56561558_56564720_56759775_56765600_RF | 17 | 699.403305 |
| 2 | ORF1_15_69599001_69607474_69822688_69824653_FF | 19 | 767.4793963 |
| 3 | ORF1_1_16764579_16768424_17044824_17050263_RR | 35 | 1194.412603 |
| 4 | ORF1_4_24971631_24973362_25125726_25129421_RR | 37 | 1204.291508 |
| 5 | ORF1_13_57155089_57159703_57172839_57181375_RR | 60 | 1450.88541 |
| 6 | ORF1_9_98247819_98251791_98368594_98373451_FF | 53 | 1421.889222 |
| 7 | ORF1_1_234729070_234732599_234814122_234817959_RF | 73 | 1642.123836 |
| 8 | ORF1_5_54687073_54701690_54895820_54899919_RF | 42 | 1300.310218 |
| 9 | ORF1_5_77219829_77221017_77232748_77239462_FR | 71 | 1610.458742 |
| 10 | ORF1_8_57997832_57999789_58156560_58160085_FF | 40 | 1277.907063 |
| 11 | ORF1_1_224094766_224099331_224328317_224334418_FF | 47 | 1350.536348 |
| 12 | ORF1_5_141346067_141348820_141380617_141383700_RR | 91 | 1843.602649 |
| 13 | ORF1_19_54789678_54792948_54890303_54892564_RF | 56 | 1442.042553 |
| 14 | ORF1_6_14206770_14210218_14395326_14400003_RR | 83 | 1813.287964 |
| 15 | ORF1_6_13871589_13875376_13978828_13980695_RR | 103 | 1968.695864 |
| 16 | ORF1_12_52899110_52901976_53003793_53005352_FF | 92 | 1844.510613 |
| 17 | ORF1_4_24971631_24973362_25161538_25167741_RR | 82 | 1797.933673 |
| 18 | ORF1_6_33937395_33940607_33982531_33984798_RF | 94 | 1906.49428 |
| 19 | ORF1_6_34087216_34091346_34298424_34301201_FR | 87 | 1821.743189 |
| 20 | ORF1_22_29467180_29469328_29549133_29556322_FR | 89 | 1839.627708 |
| 21 | ORF1_13_112592963_112600705_112765663_112769601_RF | 112 | 2050.285091 |
| 22 | ORF1_1_89127656_89129856_89152351_89155427_RR | 113 | 2053.131489 |
| 23 | ORF1_12_62435376_62437096_62567854_62572530_RR | 99 | 1932.678217 |
| 24 | ORF1_1_117001584_117004481_117080679_117085351_RF | 85 | 1818.554381 |
| 25 | ORF1_1_89127656_89129856_89172214_89177508_RR | 128 | 2141.773756 |
| 26 | ORF1_20_4141331_4145409_4240264_4242807_RF | 95 | 1907.921601 |
| 27 | ORF1_1_234593513_234598020_234817959_234822547_FR | 120 | 2105.950769 |
| 28 | ORF1_16_86891168_86894116_87066023_87067839_FF | 101 | 1940.201045 |
| 29 | ORF1_X_44917246_44925908_44940640_44941996_RR | 108 | 2034.612448 |
| 30 | ORF1_2_54954222_54956881_55045882_55048981_FF | 145 | 2359.189043 |
| 31 | ORF1_2_92108138_92110573_92242402_92244832_FF | 137 | 2251.709055 |
| 32 | ORF1_16_29013007_29015111_29280587_29285290_RF | 134 | 2210.422483 |
| 33 | ORF1_13_112765663_112769601_112883502_112886179_FR | 140 | 2289.180838 |
| 34 | ORF1_2_28424743_28426988_28597316_28599022_FF | 138 | 2263.050952 |
| 35 | ORF1_6_14018522_14021640_14102931_14109639_RR | 167 | 2550.977959 |
| 36 | ORF1_6_33846691_33848820_33982531_33984798_RF | 143 | 2295.859761 |
| 37 | ORF1_14_74374067_74376797_74492554_74493993_RF | 147 | 2363.728795 |
| 38 | ORF1_2_92108138_92110573_92210006_92223089_FF | 151 | 2380.232566 |
| 39 | ORF1_1_33081752_33087352_33257774_33262528_RR | 152 | 2434.427905 |
| 40 | ORF1_5_77940215_77942645_77971521_77974256_FF | 153 | 2437.102121 |
| 41 | ORF1_5_77232748_77239462_77325709_77329000_RF | 174 | 2605.038446 |
| 42 | ORF1_19_54627987_54632181_54748746_54753675_FF | 188 | 2692.859066 |
| 43 | ORF1_6_33982531_33984798_34087216_34091346_RF | 165 | 2550.07527 |
| 44 | ORF1_15_69554282_69555739_69599001_69607474_RF | 160 | 2523.803019 |
| 45 | ORF1_16_70112537_70113568_70359934_70360962_FR | 163 | 2539.648071 |
| 46 | ORF1_5_76987562_76988733_77232748_77239462_RR | 201 | 2796.081245 |
| 47 | ORF1_2_8316756_8318269_8515734_8517409_RF | 159 | 2511.182773 |
| 48 | ORF1_8_8984061_8987615_9203064_9212772_RR | 161 | 2528.041334 |
| 49 | ORF1_1_165695181_165697826_165952803_165953958_FF | 162 | 2535.270897 |
| 50 | ORF1_9_98247819_98251791_98381297_98383610_FR | 178 | 2639.438668 |

TABLE 6.a2

| | FC: (class1/class2) | pfp | P.value | Type |
|---|---|---|---|---|
| 1 | −1.604949366 | 0.006055278 | 7.29371E−07 | HC |
| 2 | −1.585263892 | 0.007714111 | 1.0385E−06 | HC |
| 3 | −1.57986708 | 0.021855405 | 5.41991E−06 | HC |
| 4 | −1.541181801 | 0.021309875 | 5.5866E−06 | HC |
| 5 | −1.538722995 | 0.025946264 | 1.10304E−05 | HC |
| 6 | −1.501578393 | 0.027297778 | 1.02511E−05 | HC |
| 7 | −1.500205854 | 0.033344725 | 1.72471E−05 | HC |
| 8 | −1.495061128 | 0.024870375 | 7.40111E−06 | HC |
| 9 | −1.49199903 | 0.031965768 | 1.60808E−05 | HC |
| 10 | −1.489916383 | 0.024505154 | 6.94517E−06 | HC |
| 11 | −1.478859991 | 0.02552298 | 8.49952E−06 | HC |
| 12 | −1.473734365 | 0.040465222 | 2.60909E−05 | HC |
| 13 | −1.469537812 | 0.027189805 | 1.07885E−05 | HC |
| 14 | −1.46751231 | 0.041820736 | 2.45943E−05 | HC |
| 15 | −1.459632816 | 0.045136366 | 3.29404E−05 | HC |
| 16 | −1.457176135 | 0.040095608 | 2.61366E−05 | HC |
| 17 | −1.454354906 | 0.04106626 | 2.38597E−05 | HC |
| 18 | −1.453554039 | 0.044136012 | 2.93959E−05 | HC |
| 19 | −1.452332007 | 0.040565048 | 2.50056E−05 | HC |
| 20 | −1.445931854 | 0.041057794 | 2.58911E−05 | HC |
| 21 | −1.442250604 | 0.047917611 | 3.80258E−05 | HC |
| 22 | −1.441935693 | 0.047726672 | 3.82124E−05 | HC |
| 23 | −1.440308223 | 0.043985399 | 3.08538E−05 | HC |
| 24 | −1.437292865 | 0.041261081 | 2.48499E−05 | HC |
| 25 | −1.434709807 | 0.048898615 | 4.43478E−05 | HC |
| 26 | −1.434049558 | 0.043787621 | 2.94741E−05 | HC |
| 27 | −1.431859199 | 0.049152691 | 4.17921E−05 | HC |
| 28 | −1.424180854 | 0.043712476 | 3.12818E−05 | HC |
| 29 | −1.423271831 | 0.04836415 | 3.70094E−05 | HC |
| 30 | −1.41919881 | 0.060559097 | 6.22175E−05 | HC |
| 31 | −1.416839847 | 0.054458544 | 5.2863E−05 | HC |
| 32 | −1.414210942 | 0.052181746 | 4.95437E−05 | HC |
| 33 | −1.414181385 | 0.05645962 | 5.60056E−05 | HC |
| 34 | −1.411289737 | 0.055023231 | 5.3801E−05 | HC |
| 35 | −1.41094975 | 0.068989954 | 8.16333E−05 | HC |
| 36 | −1.41068641 | 0.055840999 | 5.65789E−05 | HC |
| 37 | −1.409419005 | 0.060136926 | 6.2636E−05 | HC |
| 38 | −1.406672271 | 0.059981014 | 6.41735E−05 | HC |
| 39 | −1.404926258 | 0.064443897 | 6.9405E−05 | HC |
| 40 | −1.404080442 | 0.064267578 | 6.96705E−05 | HC |
| 41 | −1.400961136 | 0.071196654 | 8.77757E−05 | HC |
| 42 | −1.399844261 | 0.073882381 | 9.84156E−05 | HC |
| 43 | −1.399014137 | 0.069740672 | 8.15334E−05 | HC |
| 44 | −1.398599688 | 0.069385444 | 7.86599E−05 | HC |
| 45 | −1.398529753 | 0.069601611 | 8.03845E−05 | HC |
| 46 | −1.396939152 | 0.078648377 | 0.000112009 | HC |
| 47 | −1.396937941 | 0.068618829 | 7.73047E−05 | HC |
| 48 | −1.396105081 | 0.06935665 | 7.91187E−05 | HC |
| 49 | −1.396062108 | 0.069613977 | 7.99055E−05 | HC |
| 50 | −1.395891843 | 0.07282288 | 9.18445E−05 | HC |

TABLE 6.a3

| | Probe sequence |
|---|---|
| | 60 mer |
| 1 | CGCGTGATGTGTTTGAAGGTGTGTTTGTTCGAAAAAGACTTGCATTTATGCAGAGTGAGG |
| 2 | AATGAAGTCCCAGGCTTTCGTCTCACGTTCGAATAACAACCAGTTTAAAAACATACTAAA |
| 3 | AGCTCAGGCAATCCATGCTTGGAGAAAGTCGATAGATGGCAAATTTCTTGATACGGGGAG |
| 4 | GGTGAGTGGCAGATAGCTGGGAAAACATTCGAGCCAAGCTGAGTGACAGCTTTGTCACGG |
| 5 | TCAGTCTAGAACATAACAGAAAATGTCTTCGACTCTTTTTACAAAAGGCCACTTGACATC |
| 6 | CAGGAGGGTAGCCCATTGGGTACCTAGCTCGAGTTCCAGGCTTAGCTCTGGAGCTGTGTG |
| 7 | AGCTCAGGCAATCCATGCTTGGAGAAAGTCGATTTCCCCATTTTGGCCGATGGCATCGCT |
| 8 | ATTAGGAGTGGGGGCCTTGAGAGGTAATTCGATCTTTTCAAAGAACCACATTTTAGTTTT |
| 9 | ATTTTATCTCCAGGTAAAAAGGGGTGTGTCGAAGTGCTCTGTGGTGATGCAGCAGGTTGA |
| 10 | AGCGAATTTAAGGCAGTAATAAGTGCAGTCGAGGCAGTGGGAGTTGAATCCTCTCCCCTC |
| 11 | GGCAGGTGGATTGCTTGAGCTCAGGAGTTCGAAAAAGAAAAGAAAAGAACATAACAGAAC |
| 12 | GTCCGAATCGTTGGGTTTGGCGGAGGGCTCGATAACTTAGAAAATGTGAATGCTAGAGAC |
| 13 | AAGGCAGGCAGATCACGAGGCCAGGAGTTCGATAGATAATAGATAGAAATATGCAGAAAG |
| 14 | AGTTTTTTCATCTGAAAGACAAACGAGGTCGAGCTGCCGTGTGTCCCATAGCCACCAGCT |
| 15 | TCTAGCTCAGGGTTTGTGAATGCACCAATCGAGGTGAACTGTGGCAATGAAAACCAGAAA |
| 16 | CTGCCGCCTAAGGTTGTTGATGTAGCTCTCGAACGTCTTGTGTAAGAATGGTTTTAATGA |
| 17 | GGTGAGTGGCAGATAGCTGGGAAAACATTCGATAACCCCAAATTTTCCAGCCCCTCACCC |
| 18 | GTTCAAATCAAGCTAGGAGGCATCCGTGTCGAAAGAAAAAGATACAGAGAGTTCCTATAT |
| 19 | GATGGCATCAGGGTACGAGGGGGAGACCTCGACACCAACATATATCTGTGATTCACTCTG |
| 20 | CAGGCAGGCAGAATGCACCATCAATGAATCGATTCTGATGACATGGTTGTTCCTCCTGTT |
| 21 | GCCGGCCCATAGCTCAGTCATGTGAACCTCGAAAAAGAAAGAAACATTTCAAAATATTGA |
| 22 | TTTAGCTCAAGGTTTGTGAATGCACCAATCGAACAGTCCAAATAAAGTCTGGAAAGAAAC |
| 23 | CGCAGCAGTTCGCTTAGGGATGCAGACGTCGAATAGTCCACTGTTACTGGCTTACTGCTT |
| 24 | GGCAGGCAGATCACTTGAGCTCAGGAGTTCGATATCTGTTTACTTAACCACAAAGATAAC |
| 25 | TTTAGCTCAAGGTTTGTGAATGCACCAATCGATATTTTACTTAACCATAGAACAATTACT |
| 26 | TGATCTGCCCAACAAGAGAGAGCTGGGATCGAATGCAACATTCATTTACTACATGACCAT |
| 27 | CTATACATCCTCCAAGGTGATGATAACTTCGAGAGTGTTTTTAGGAGGCTGGTGCTGACA |
| 28 | ATTCATAGGCACGGGTACCAAGACTACTTCGATGCTGCATTTTAGAACATTCCAGCAAAA |
| 29 | GGCAGGCAGATCACTTGAGGTCAAGAGTTCGAGTCAAAGTGATGCTCCATGAAAAAAGAG |
| 30 | TAACAGGTTTAAAACAAAAATTTAAAACTCGAGGGGGCGTAGTTCTGCCGGGGTTTCAGC |
| 31 | TGGAAACGGGATATCTTCATATAAAATCTCGAAATATCCTCTTGCAGATATTACAAAAAG |
| 32 | AGAGGTGTCCTCCGTGGGTCAGGAAGGGTCGACAATCATGTTGAGCAAATGAATTAAAAA |
| 33 | GCCGGCCCATAGCTCAGTCATGTGAACCTCGACAGTAGGTATTTTTGGAAGCTCAGATTT |
| 34 | GGCAGGAGGATCACTTGAGGCCAAGAATTCGATTCGTCCAATTATGAGAACATGCTGCAC |
| 35 | TTACAAACCTTGAGCTAGATACAGAGTGTCGAAATCAGCTTGAGGTCTTGGGGACTGAGT |
| 36 | GTTCAAATCAAGCTAGGAGGCATCCGTGTCGAATTTTCATGGCAGCCCTCTTTTGATATC |
| 37 | CGCCAGGTTTAGCGTGAGGCGCTCCAGGTCGAGTGGTTGACCTGGAAGCAAAATTAAAAG |
| 38 | TGGAAACGGGATATCTTCATATAAAATCTCGAAATATCCTCTTGCAGATTTTACAAAAAG |
| 39 | GTCTCAGCCTAAGAGCAAGAGCTTAAGTTCGAGCAGTGTGGAACCTTCGGGAGCTGTGTG |
| 40 | GTGGGCAGATGACTTGAGCTTCAGGAGTTCGAGACCAGTTTGGCCAACAAAGCAAAACCG |
| 41 | GTTTGACTTGGAGCCTTCTCATTGCAGGTCGAAGTGCTCTGTGGTGATGCAGCAGGTTGA |
| 42 | GTGGTGGTACAGGTGCTATGCTTATGACTCGAGATCTCCACTCAGATACCAAAATCTGTA |
| 43 | GATGGCATCAGGGTACGAGGGGGAGACCTCGATGAGCAGCATGTTATTTACTGGCTCCTG |
| 44 | AATGAAGTCCCAGGCTTTCGTCTCACGTTCGACAGCCTAAATCTCTGAAGCCGTCTCCTG |
| 45 | TAAAGCTATGCATAAGACTAAGGTTCTGTCGAACCACCTTTATTTACTGTATATCAGCTA |
| 46 | CACTGCACTCATCAATAACATCTTCAACTCGAAGTGCTCTGTGGTGATGCAGCAGGTTGA |
| 47 | GGCAGGTGGATCACTTGAGCTCAGGAGTTCGAAACCCAACATAAATGCAAATAAATAAAT |
| 48 | GGCAGGTGGATCACTTGAGGCCACGAGTTCGAGTACTCAGATATTTACTTACCTACTCAA |
| 49 | GGCGGGAGGATCACCTAAGCTCAGGAGTTCGAGACTAGCTTGGCCAACATGGTAAAATCC |
| 50 | CAGGAGGGTAGCCCATTGGGTACCTAGCTCGAATGCAGGCTGTTGTTGGAAGATCCGTGA |

TABLE 6.a4

| | Probe Location | | | | | 4 kb Sequence Location | | | | |
|---|---|---|---|---|---|---|---|---|---|---|
| | | Start 1 | End1 | Start2 | End2 | | Start1 | End1 | Start2 | End2 |
| 1 | 19 | 56561560 | 56561589 | 56765569 | 56765598 | 19 | 56561560 | 56565559 | 56761599 | 56765598 |
| 2 | 15 | 69607443 | 69607472 | 69824622 | 69824651 | 15 | 69603473 | 69607472 | 69820652 | 69824651 |
| 3 | 1 | 16764581 | 16764610 | 17044826 | 17044855 | 1 | 16764581 | 16768580 | 17044826 | 17048825 |
| 4 | 4 | 24971633 | 24971662 | 25125728 | 25125757 | 4 | 24971633 | 24975632 | 25125728 | 25129727 |
| 5 | 13 | 57155091 | 57155120 | 57172841 | 57172870 | 13 | 57155091 | 57159090 | 57172841 | 57176840 |
| 6 | 9 | 98251760 | 98251789 | 98373420 | 98373449 | 9 | 98247790 | 98251789 | 98369450 | 98373449 |
| 7 | 1 | 234729072 | 234729101 | 234817928 | 234817957 | 1 | 234729072 | 234733071 | 234813958 | 234817957 |
| 8 | 5 | 54687075 | 54687104 | 54899888 | 54899917 | 5 | 54687075 | 54691074 | 54895918 | 54899917 |
| 9 | 5 | 77220986 | 77221015 | 77232750 | 77232779 | 5 | 77217016 | 77221015 | 77232750 | 77236749 |
| 10 | 8 | 57999758 | 57999787 | 58160054 | 58160083 | 8 | 57995788 | 57999787 | 58156084 | 58160083 |
| 11 | 1 | 224099300 | 224099329 | 224334387 | 224334416 | 1 | 224095330 | 224099329 | 224330417 | 224334416 |
| 12 | 5 | 141346069 | 141346098 | 141380619 | 141380648 | 5 | 141346069 | 141350068 | 141380619 | 141384618 |
| 13 | 19 | 54789680 | 54789709 | 54892533 | 54892562 | 19 | 54789680 | 54793679 | 54888563 | 54892562 |
| 14 | 6 | 14206772 | 14206801 | 14395328 | 14395357 | 6 | 14206772 | 14210771 | 14395328 | 14399327 |
| 15 | 6 | 13871591 | 13871620 | 13978830 | 13978859 | 6 | 13871591 | 13875590 | 13978830 | 13982829 |
| 16 | 12 | 52901945 | 52901974 | 53005321 | 53005350 | 12 | 52897975 | 52901974 | 53001351 | 53005350 |
| 17 | 4 | 24971633 | 24971662 | 25161540 | 25161569 | 4 | 24971633 | 24975632 | 25161540 | 25165539 |
| 18 | 6 | 33937397 | 33937426 | 33984767 | 33984796 | 6 | 33937397 | 33941396 | 33980797 | 33984796 |
| 19 | 6 | 34091315 | 34091344 | 34298426 | 34298455 | 6 | 34087345 | 34091344 | 34298426 | 34302425 |
| 20 | 22 | 29469297 | 29469326 | 29549135 | 29549164 | 22 | 29465327 | 29469326 | 29549135 | 29553134 |
| 21 | 13 | 112592965 | 112592994 | 112769570 | 112769599 | 13 | 112592965 | 112596964 | 112765600 | 112769599 |
| 22 | 1 | 89127658 | 89127687 | 89152353 | 89152382 | 1 | 89127658 | 89131657 | 89152353 | 89156352 |
| 23 | 12 | 62435378 | 62435407 | 62567856 | 62567885 | 12 | 62435378 | 62439377 | 62567856 | 62571855 |
| 24 | 1 | 117001586 | 117001615 | 117085320 | 117085349 | 1 | 117001586 | 117005585 | 117081350 | 117085349 |
| 25 | 1 | 89127658 | 89127687 | 89172216 | 89172245 | 1 | 89127658 | 89131657 | 89172216 | 89176215 |
| 26 | 20 | 4141333 | 4141362 | 4242776 | 4242805 | 20 | 4141333 | 4145332 | 4238806 | 4242805 |
| 27 | 1 | 234597989 | 234598018 | 234817961 | 234817990 | 1 | 234594019 | 234598018 | 234817961 | 234821960 |
| 28 | 16 | 86894085 | 86894114 | 87067808 | 87067837 | 16 | 86890115 | 86894114 | 87063838 | 87067837 |
| 29 | X | 44917248 | 44917277 | 44940642 | 44940671 | X | 44917248 | 44921247 | 44940642 | 44944641 |
| 30 | 2 | 54956850 | 54956879 | 55048950 | 55048979 | 2 | 54952880 | 54956879 | 55044980 | 55048979 |
| 31 | 2 | 92110542 | 92110571 | 92244801 | 92244830 | 2 | 92106572 | 92110571 | 92240831 | 92244830 |
| 32 | 16 | 29013009 | 29013038 | 29285259 | 29285288 | 16 | 29013009 | 29017008 | 29281289 | 29285288 |
| 33 | 13 | 112769570 | 112769599 | 112883504 | 112883533 | 13 | 112765600 | 112769599 | 112883504 | 112887503 |
| 34 | 2 | 28426957 | 28426986 | 28598991 | 28599020 | 2 | 28422987 | 28426986 | 28595021 | 28599020 |
| 35 | 6 | 14018524 | 14018553 | 14102933 | 14102962 | 6 | 14018524 | 14022523 | 14102933 | 14106932 |
| 36 | 6 | 33846693 | 33846722 | 33984767 | 33984796 | 6 | 33846693 | 33850692 | 33980797 | 33984796 |
| 37 | 14 | 74374069 | 74374098 | 74493962 | 74493991 | 14 | 74374069 | 74378068 | 74489992 | 74493991 |
| 38 | 2 | 92110542 | 92110571 | 92223058 | 92223087 | 2 | 92106572 | 92110571 | 92219088 | 92223087 |
| 39 | 1 | 33081754 | 33081783 | 33257776 | 33257805 | 1 | 33081754 | 33085753 | 33257776 | 33261775 |
| 40 | 5 | 77942614 | 77942643 | 77974225 | 77974254 | 5 | 77938644 | 77942643 | 77970255 | 77974254 |
| 41 | 5 | 77232750 | 77232779 | 77328969 | 77328998 | 5 | 77232750 | 77236749 | 77324999 | 77328998 |
| 42 | 19 | 54632150 | 54632179 | 54753644 | 54753673 | 19 | 54628180 | 54632179 | 54749674 | 54753673 |
| 43 | 6 | 33982533 | 33982562 | 34091315 | 34091344 | 6 | 33982533 | 33986532 | 34087345 | 34091344 |
| 44 | 15 | 69554284 | 69554313 | 69607443 | 69607472 | 15 | 69554284 | 69558283 | 69603473 | 69607472 |
| 45 | 16 | 70113537 | 70113566 | 70359936 | 70359965 | 16 | 70109567 | 70113566 | 70359936 | 70363935 |
| 46 | 5 | 76987564 | 76987593 | 77232750 | 77232779 | 5 | 76987564 | 76991563 | 77232750 | 77236749 |
| 47 | 2 | 8316758 | 8316787 | 8517378 | 8517407 | 2 | 8316758 | 8320757 | 8513408 | 8517407 |
| 48 | 8 | 8984063 | 8984092 | 9203066 | 9203095 | 8 | 8984063 | 8988062 | 9203066 | 9207065 |
| 49 | 1 | 165697795 | 165697824 | 165953927 | 165953956 | 1 | 165693825 | 165697824 | 165949957 | 165953956 |
| 50 | 9 | 98251760 | 98251789 | 98381299 | 98381328 | 9 | 98247790 | 98251789 | 98381299 | 98385298 |

TABLE 6.a5

| | probe | Primer ID |
|---|---|---|
| 1 | ORF1_19_56561558_56564720_56759775_56765600_RF | OBD168-1529 |
| 2 | ORF1_15_69599001_69607474_69822688_69824653_FF | OBD168-681 |
| 3 | ORF1_1_16764579_16768424_17044824_17050263_RR | OBD168-921 |
| 4 | ORF1_4_24971631_24973362_25125726_25129421_RR | OBD168-893 |
| 5 | ORF1_13_57155089_57159703_57172839_57181375_RR | OBD168-1273 |
| 6 | ORF1_9_98247819_98251791_98368594_98373451_FF | OBD168-617 |
| 7 | ORF1_1_234729070_234732599_234814122_234817959_RF | OBD168-633 |
| 8 | ORF1_5_54687073_54701690_54895820_54899919_RF | OBD168-1629 |
| 9 | ORF1_5_77219829_77221017_77232748_77239462_FR | OBD168-1145 |
| 10 | ORF1_8_57997832_57999789_58156560_58160085_FF | OBD168-885 |
| 11 | ORF1_1_224094766_224099331_224328317_224334418_FF | OBD168-1741 |
| 12 | ORF1_5_141346067_141348820_141380617_141383700_RR | OBD168-517 |
| 13 | ORF1_19_54789678_54792948_54890303_54892564_RF | OBD168-1525 |
| 14 | ORF1_6_14206770_14210218_14395326_14400003_RR | OBD168-789 |
| 15 | ORF1_6_13871589_13875376_13978828_13980695_RR | OBD168-1653 |
| 16 | ORF1_12_52899110_52901976_53003793_53005352_FF | OBD168-689 |
| 17 | ORF1_4_24971631_24973362_25161538_25167741_RR | OBD168-457 |
| 18 | ORF1_6_33937395_33940607_33982531_33984798_RF | OBD168-793 |
| 19 | ORF1_6_34087216_34091346_34298424_34301201_FR | OBD168-2033 |
| 20 | ORF1_22_29467180_29469328_29549133_29556322_FR | OBD168-1961 |
| 21 | ORF1_13_112592963_112600705_112765663_112769601_RF | OBD168-1825 |
| 22 | ORF1_1_89127656_89129856_89152351_89155427_RR | OBD168-1317 |
| 23 | ORF1_12_62435376_62437096_62567854_62572530_RR | OBD168-1381 |
| 24 | ORF1_1_117001584_117004481_117080679_117085351_RF | OBD168-1077 |
| 25 | ORF1_1_89127656_89129856_89172214_89177508_RR | OBD168-1321 |
| 26 | ORF1_20_4141331_4145409_4240264_4242807_RF | OBD168-1213 |
| 27 | ORF1_1_234593513_234598020_234817959_234822547_FR | OBD168-589 |
| 28 | ORF1_16_86891168_86894116_87066023_87067839_FF | OBD168-869 |
| 29 | ORF1_X_44917246_44925908_44940640_44941996_RR | OBD168-1717 |
| 30 | ORF1_2_54954222_54956881_55045882_55048981_FF | OBD168-1909 |
| 31 | ORF1_2_92108138_92110573_92242402_92244832_FF | OBD168-1547 |
| 32 | ORF1_16_29013007_29015111_29280587_29285290_RF | OBD168-745 |
| 33 | ORF1_13_112765663_112769601_112883502_112886179_FR | OBD168-1405 |
| 34 | ORF1_2_28424743_28426988_28597316_28599022_FF | OBD168-1017 |
| 35 | ORF1_6_14018522_14021640_14102931_14109639_RR | OBD168-1081 |
| 36 | ORF1_6_33846691_33848820_33982531_33984798_RF | OBD168-525 |
| 37 | ORF1_14_74374067_74376797_74492554_74493993_RF | OBD168-521 |
| 38 | ORF1_2_92108138_92110573_92210006_92223089_FF | OBD168-1546 |
| 39 | ORF1_1_33081752_33087352_33257774_33262528_RR | OBD168-877 |
| 40 | ORF1_5_77940215_77942645_77971521_77974256_FF | OBD168-2017 |
| 41 | ORF1_5_77232748_77239462_77325709_77329000_RF | OBD168-1009 |
| 42 | ORF1_19_54627987_54632181_54748746_54753675_FF | OBD168-1517 |
| 43 | ORF1_6_33982531_33984798_34087216_34091346_RF | OBD168-1661 |
| 44 | ORF1_15_69554282_69555739_69599001_69607474_RF | OBD168-965 |
| 45 | ORF1_16_70112537_70113568_70359934_70360962_FR | OBD168-1417 |
| 46 | ORF1_5_76987562_76988733_77232748_77239462_RR | OBD168-777 |
| 47 | ORF1_2_8316756_8318269_8515734_8517409_RF | OBD168-785 |
| 48 | ORF1_8_8984061_8987615_9203064_9212772_RR | OBD168-1189 |
| 49 | ORF1_1_165695181_165697826_165952803_165953958_FF | OBD168-1329 |
| 50 | ORF1_9_98247819_98251791_98381297_98383610_FR | OBD168-1701 |

TABLE 6.a6

| | Sequence | Primer ID | Sequence |
|---|---|---|---|
| 1 | CAAGTGTGGGGGCGAGTGTTGCG | OBD168-1531 | TGCACACCCAGGACCTGGAAAAA |
| 2 | CCGTTGCTCAAGAACCATCCAGAGTC | OBD168-683 | GCAAACTTCAGGCTGTATTGTTCTCG |
| 3 | TTATCCCCTGTCTGACTTGTTCCCCG | OBD168-923 | GCGTTACATCTGTTGTGCCAGCAAAA |
| 4 | CAGTAGTTCTAAAAGAGAGGATGGTA | OBD168-895 | GCCAGTAAACCAAGGCTTATTAGGAT |
| 5 | GCGGGAGACAGAGAAAGGAAAAG | OBD168-1275 | CAGGCAGAGGTGGACCGAGAACC |
| 6 | CATTCCCAGCAGGAGGGTAAAGGCTT | OBD168-619 | ACAACAACAATAACAGACACCAGGGT |
| 7 | TATCCCCTGTCTGACTCGTTCCC | OBD168-635 | TTTGGAATGCGGTGGCGTCTGGC |
| 8 | TGGAAAGGACTCCCAAATTCA | OBD168-1631 | TAAAATCAGTAATCTAAGCTT |
| 9 | GCTACATCTGCTCAACCGTCCTG | OBD168-1147 | CCCACACTGCCAAGCCCTTACCA |
| 10 | AACATCAGTCAGGAAAGACAAGGTTC | OBD168-887 | TGGTGTGTCCTGGGTTTTGTGGAGCA |
| 11 | AAAGTGAGAGAAGGGTCCTGTAGCA | OBD168-1743 | TTCACATGTTCAGTGTCTTCCTTTT |
| 12 | TGGCTGATGAGCGTGTCCGCATAGTT | OBD168-519 | CTCCGAACACAAAGCATACATACCCT |
| 13 | AAAGTAATATCTTCCCAATGACC | OBD168-1527 | TCTGACCTCACTTTCTCACCTCT |
| 14 | AAGTATGATGTAGAAAAGCCTTGAGC | OBD168-791 | ATGGATGGGCAGTGGAAATAAGGCAG |
| 15 | CTACTCTGGTGGGACCTTGGA | OBD168-1655 | GTCTAGACTACAACACAAAGT |
| 16 | GGGTGGGAGTTGCTCACTTGTTC | OBD168-691 | GCCTGGCACACGGTGGGAAACAA |
| 17 | CAGTAGTTCTAAAAGAGAGGATGGTA | OBD168-459 | CCAGGAAACAGGAAGAGTTCTCAAGC |
| 18 | TGGAAGTGCTGAACTGGAGCCCC | OBD168-795 | GAGGAATAGGCAGAGCACAGCAG |
| 19 | GCCACTTATCACTGGGGACACCTGAA | OBD168-2035 | ACATCATTAGTTTCTACATTCTACAA |
| 20 | ACTCCCTAAAATCAAACAACA | OBD168-1963 | AGATGCTTCCCTGTTGTATTA |
| 21 | CCTCTTTGGCCAGAGCCAAAAC | OBD168-1827 | CTTGATTTGGTTTTCCTTTTCT |
| 22 | ATTGTAAACACACCAATCGGC | OBD168-1319 | TCCTTCCTTAGCTGAGATCCC |
| 23 | ATGGCACCTGCCGCAGCTCAGGTGGG | OBD168-1383 | TGTATCTCAATGGATTCTCTCATTAT |
| 24 | GGGTAACTTGTAGACACTGCTGCCTC | OBD168-1079 | GCCCACTGAGAAATAGAGATGTCTTG |
| 25 | TGTGTATCTAGCTGCTCTGGTGGG | OBD168-1323 | TGGATTGGATCATGAAGCAGAAGG |
| 26 | AGGTGGAGGTCAGTGATGCTCGG | OBD168-1215 | GAACATCCCTCACTTGCCCAGTC |
| 27 | TGGTGGTTCATAGGCAGGCTGAG | OBD168-591 | ATGATGGCTGTGCCTGGGTGCTG |
| 28 | ATGGAGGCTGCCCCTGTAACACA | OBD168-871 | TGTGTTACCCCTGCTCCCACGGA |
| 29 | AAGGTATGTGTGTGTTGAGGGGTGGG | OBD168-1719 | ATCCGCCCACCTCGGCCTCCCAAAGT |
| 30 | CCAGCTACATCAAAACCCACCAC | OBD168-1911 | TGGCAAGGAATCCACAGGTTTTC |
| 31 | TCTCCAAGGCACTCACAAGTAT | OBD168-1549 | GAAACGTTCATCAGGAGTTGGA |
| 32 | TTGTGAGTGGGAAGCAGTGGGATTTC | OBD168-747 | GTGTTAGGGCTGGAAGGAACTTGTTG |
| 33 | CTGCACACTGGAGCAGGGACCTTGG | OBD168-1407 | ACCCTCATCTTTGGTGTGATGTTTG |
| 34 | AAGGAATAGGAGGCTGAGTATGGTGG | OBD168-1019 | GGTTCAACTCCTGACCTTGCCTTCAT |
| 35 | GGTAACAGAGTGTGGACTGGTGC | OBD168-1083 | TCAGGCAGGACCAAGACCACCCA |
| 36 | TGGAAGTGCTGAACTGGAGCCCC | OBD168-527 | CCAGTATCCTGACTCCTGACAGC |
| 37 | TTGTGGAGGAAGATGGCTGCCGC | OBD168-523 | TATGGTGGAATGGACCTGCCTGC |
| 38 | TCTCCAAGGCACTCACAAGTAT | OBD168-1548 | GAAACGTTCATCAGGAGTTGGA |
| 39 | CCAGTCTTGACCACGCCCACCTA | OBD168-879 | TTGGGCGAGATTCCAGCCTACAC |
| 40 | CACTTCAATTGAAAAAAAAAAAAG | OBD168-2019 | CGCCCGGCTAAGTTTTCTGCAGTT |
| 41 | GTCCTAATCCACAAGGGCATCTTTCT | OBD168-1011 | TCAAAGGATTCGCTACTTACAGCAGT |
| 42 | ACTCACAGGTGGCATTTGATGGC | OBD168-1519 | TCAAAAATATTCAGAGAAAATAT |
| 43 | TGTTATCGCTCAGCCACTTATCACTG | OBD168-1663 | CCACTTTCCTTTCCTTCCCACCCCAG |
| 44 | ACAATCTGCTCTCCACCACCAGC | OBD168-967 | GCCAAGGTCGCTGGAGATGTGAG |
| 45 | GGTTTGTGTGGTGAGACAGTATT | OBD168-1419 | AGGCTGGTCTTGAACTCCTAATC |
| 46 | GACACCAGTGATTGGATTTCAGGTTC | OBD168-779 | TCAAAGGATTCGCTACTTACAGCAGT |
| 47 | CCCGTATTCAGAGACCACCCACTTTA | OBD168-787 | TCCTGAGGGCTTCATTCAAACTTG |
| 48 | CTGGGAGTGGTAGATGTAATGCCTGT | OBD168-1191 | GCCTCTAATAAGTGTGGAATCCTGGG |
| 49 | AATTCCTCTCCTATGCACACATT | OBD168-1331 | TCAAGCGATTCTTCTGCCTCAGC |
| 50 | TAGCAGAGGGCTCCTCCCGCTCAT | OBD168-1703 | TGGGTTGTATTCCACCTGCTATTC |

TABLE 6.a7

| | qPCR Primer ID | Sequence | qPCR Primer ID |
|---|---|---|---|
| 1 | OBD168-q1529 | GTGGGGGCGAGTGTTGCGTGAG | OBD168-q1531 |
| 2 | OBD168-q681 | GCCTCTGCCCCACTGCTCAGGAG | OBD168-q683 |
| 3 | OBD168-q921 | CTGGTGAGGAAGGAGGAAGAGAGGAGGC | OBD168-q923 |
| 4 | OBD168-q893 | GAAAGGTGGGACAACTGGAAGTGAGAGC | OBD168-q895 |
| 5 | OBD168-q1273 | AGAGAGCGGGAGACAGAGAAAGGAAAAG | OBD168-q1275 |
| 6 | OBD168-q617 | CTCATTCCCAGCAGGAGGGTAAAGGCTT | OBD168-q619 |
| 7 | OBD168-q633 | CTGGTGAGGAAGGAGGAAGAGAGGAGGC | OBD168-q635 |
| 8 | OBD168-q1629 | GTTATGGACCAAATGTCTGGCC | OBD168-q1631 |
| 9 | OBD168-q1145 | GAGGAAGTGGAGGCACAGGCGGCTCAAG | OBD168-q1147 |
| 10 | OBD168-q885 | CCTCACAGCCCCATTAGAAGTTTATTTC | OBD168-q887 |
| 11 | OBD168-q1741 | AAAAAAATAAAAAATAGTTAAT | OBD168-q1743 |
| 12 | OBD168-q517 | GCCAGGAAGACGCAGGAGACCGC | OBD168-q519 |
| 13 | OBD168-q1525 | CAAGCATGAGTGACAACCAAT | OBD168-q1527 |
| 14 | OBD168-q789 | CAGTGTCATAGAGTCCAAAGTTGTAGGC | OBD168-q791 |
| 15 | OBD168-q1653 | TCAGGGATTGTAAATACACCAAT | OBD168-q1655 |
| 16 | OBD168-q689 | CGGTGACTTCAGTTGGGTGGAGGGTGGG | OBD168-q691 |
| 17 | OBD168-q457 | GAAAGGTGGGACAACTGGAAGTGAGAGC | OBD168-q459 |
| 18 | OBD168-q793 | CAGGAGGACGGCAGGGCTATGGACCTA | OBD168-q795 |
| 19 | OBD168-q2033 | CCTGCTAGGTGGGAGTCACCTG | OBD168-q2035 |
| 20 | OBD168-q1961 | AACATTCATGTCAAAACTCCCTAA | OBD168-q1963 |
| 21 | OBD168-q1825 | CCTCTTTGGCCAGAGCCAAAACC | OBD168-q1827 |
| 22 | OBD168-q1317 | TTTATGTCTAGCTCAGGGATTG | OBD168-q1319 |
| 23 | OBD168-q1381 | CAGCTCAGGTGGGGTGTGGTG | OBD168-q1383 |
| 24 | OBD168-q1077 | GGCATCAGGGTGGCAGTGGCAGGC | OBD168-q1079 |
| 25 | OBD168-q1321 | GGGGCCTTGGAGAACCTTTATG | OBD168-q1323 |
| 26 | OBD168-q1213 | GGCTGGAGTGGGACATTGGCTGGGCTGT | OBD168-q1215 |
| 27 | OBD168-q589 | ATTTTGGTGGTTCATAGGCAGGCTGAGC | OBD168-q591 |
| 28 | OBD168-q869 | AGCCTCCCTGCCCATCTGCCCCTCTCAT | OBD168-q871 |
| 29 | OBD168-q1717 | AAAGATTCATGCTCTCTCAAGG | OBD168-q1719 |
| 30 | OBD168-q1909 | GAAAAAAATAGGTGTGTGAATAAT | OBD168-q1911 |
| 31 | OBD168-q1547 | GGGTGGGGTGCCTGGATGCGTGAGG | OBD168-q1549 |
| 32 | OBD168-q745 | CCTGGATTTGAACTGCCTTGTCCGTGGG | OBD168-q747 |
| 33 | OBD168-q1405 | GTTCTCTTCTGCACACTGGAGCAG | OBD168-q1407 |
| 34 | OBD168-q1017 | AAAGGAATAGGAGGCTGAGTATGGTGGC | OBD168-q1019 |
| 35 | OBD168-q1081 | GCTGGGTAACAGAGTGTGGACTGGTGC | OBD168-q1083 |
| 36 | OBD168-q525 | GGCAACCAGGAGGACGGCAGGGC | OBD168-q527 |
| 37 | OBD168-q521 | TTGTGGAGGAAGATGGCTGCCGCCTGGG | OBD168-q523 |
| 38 | OBD168-q1546 | GGGTGGGGTGCCTGGATGCGTGAGG | OBD168-q1548 |
| 39 | OBD168-q877 | CCCCAGTCTTGACCACGCCCACC | OBD168-q879 |
| 40 | OBD168-q2017 | CACTGAATTGTTCAGTTTAAA | OBD168-q2019 |
| 41 | OBD168-q1009 | CTGATAAGAAGAGGATTGTAGGACTGCC | OBD168-q1011 |
| 42 | OBD168-q1517 | ACTCAGGAGGGAATGTAACCCTC | OBD168-q1519 |
| 43 | OBD168-q1661 | GCTCCCGGGCCCTGCTAGGTGGG | OBD168-q1663 |
| 44 | OBD168-q965 | CTTCCCGCTCTGCTCTTTTGAGGCTGGC | OBD168-q967 |
| 45 | OBD168-q1417 | CAGTATTGAATCCAACAAACGG | OBD168-q1419 |
| 46 | OBD168-q777 | GGGCTCCTCTGTGTATCTCCTCTGTCTC | OBD168-q779 |
| 47 | OBD168-q785 | CCCCGCAAAAGGCTTGAAGGAGCACCCG | OBD168-q787 |
| 48 | OBD168-q1189 | GTCTCCACGGTCTCTGCTGTCTCCTTGC | OBD168-q1191 |
| 49 | OBD168-q1329 | ATTCCCAGCACAAATGATTATCTTCC | OBD168-q1331 |
| 50 | OBD168-q1701 | TAGGACTAGGATTTGAACCCAGGC | OBD168-q1703 |

TABLE 6.a8

| | Sequence | qPCR Probe | Sequence |
|---|---|---|---|
| 1 | GGTCATCTAGTCACATCTCATA | OBD168-p1529 | GTTTGAAGGTGTGTTTGTTCGAAAAAGACTTGCATTTATG |
| 2 | GCAAACTTCAGGCTGTATTGTTCTCGTC | OBD168-p681 | AGTCCCAGGCTTTCGTCTCACGTTCGAATAACAACC |
| 3 | CCAGCGTTACATCTGTTGTGCCAGCAAA | OBD168-p921 | TCAGGCAATCCATGCTTGGAGAAAGTCGATAGATGGC |
| 4 | GCCAGTAAACCAAGGCTTATTAGGATAC | OBD168-p893 | AGTGGCAGATAGCTGGGAAAACATTCGAGCCAAGC |
| 5 | TCAGGCAGAGGTGGACCGAGAACC | OBD168-p1273 | AGTTCAGTCTAGAACATAACAGAAAATGTCTTCGACTCT |
| 6 | GGGACTTGACAGTTCATCTCATTCATTC | OBD168-p617 | AGGGTAGCCCATTGGGTACCTAGCTCGAGTTC |
| 7 | CCTGTTTTGGAATGCGGTGGCGTCTGGC | OBD168-p633 | TGGAGAAAGTCGATTTCCCCATTTTGGCCGATGGC |
| 8 | TTCCGTTTTAGGAAACTAGAAA | OBD168-p1629 | GGGGCCTTGAGAGGTAATTCGATCTTTTCAAAGAACCACA |
| 9 | CTCCCCACACTGCCAAGCCCTTACC | OBD168-p1147 | TGCATCACCACAGAGCACTTCGACACACCCCT |
| 10 | TGGTGTGTCCTGGGTTTTGTGGAGCAC | OBD168-p885 | AGTGCAGTCGAGGCAGTGGGAGTTGAATCCTC |
| 11 | TTCTAGTTTGGACAATGTATGA | OBD168-p1741 | TTGCTTGAGCTCAGGAGTTCGAAAAAGAAAAGAAAAGAAC |
| 12 | CTACTCCGAACACAAAGCATACATACCC | OBD168-p517 | TCGTTGGGTTTGGCGGAGGGCTCGATAACTTAG |
| 13 | TTTCTCACCTCTAGATATGTT | OBD168-p1525 | GATCACGAGGCCAGGAGTTCGATAGATAATAGATAGAAAT |
| 14 | GCTGCCTGCTGTTCCCGTGCCAG | OBD168-p789 | AAAGACAAACGAGGTCGAGCTGCCGTGTGTCC |
| 15 | TTTTCAAGCAGGGCTTCAGCTTT | OBD168-p1653 | GGTTTGTGAATGCACCAATCGAGGTGAACTGTGGCAATGA |
| 16 | TACCCGCAAGTGTCTGTGCCTGGC | OBD168-p689 | TGCCGCCTAAGGTTGTTGATGTAGCTCTCGAACGT |
| 17 | CAGACCAGTATTCCGACAGCAGGCTTGC | OBD168-p457 | TGAGTGGCAGATAGCTGGGAAAACATTCGATAACCCC |
| 18 | GGAGGGAGGAATAGGCAGAGCACAGCAG | OBD168-p793 | AGTTCAAATCAAGCTAGGAGGCATCCGTGTCGAAAGA |
| 19 | TACATTCTACAAGGTGAAAGAC | OBD168-p2033 | GGGTACGAGGGGGAGACCTCGACACCAACATATATCTGTG |
| 20 | TGAGAGATGCTTCCCTGTTGTATT | OBD168-p1961 | GAATGCACCATCAATGAATCGATTCTGATGACATGGTTGT |
| 21 | AGCTACCGCGCCCAGCATCCATT | OBD168-p1825 | AGCTCAGTCATGTGAACCTCGAAAAAGAAAGAAACATTTC |
| 22 | TTTCTAAGTGAGATGTGGAAAT | OBD168-p1317 | GGTTTGTGAATGCACCAATCGAACAGTCCAAATAAAGTCT |
| 23 | GGAACTCAATTTTTATTCAGC | OBD168-p1381 | CGCTTAGGGATGCAGACGTCGAATAGTCCACTGTTACTGG |
| 24 | GAGGTTGAGTGACAAATGCTGTGCTCTC | OBD168-p1077 | AGCTCAGGAGTTCGATATCTGTTTACTTAACCACAAA |
| 25 | ACTACAGAAACATAATGACTAA | OBD168-p1321 | GGTTTGTGAATGCACCAATCGATATTTTACTTAACCATAG |
| 26 | CCAGCACCCAGAGGCAAACTCACCC | OBD168-p1213 | TCTGCCCAACAAGAGAGAGCTGGGATCGAATGC |
| 27 | GCCCCAGGTGCCAGCAGGGTGAC | OBD168-p589 | ACTATACATCCTCCAAGGTGATGATAACTTCGAGAGTG |
| 28 | GACTGTGTTACCCCTGCTCCCACGG | OBD168-p869 | AGGCACGGGTACCAAGACTACTTCGATGCTGC |
| 29 | TGGTCAGGCTGGTCTCCAACTC | OBD168-p1717 | TCACTTGAGGTCAAGAGTTCGAGTCAAAGTGATGCTCCAT |
| 30 | TTTAGGAATGTCAACCATTTTGCC | OBD168-p1909 | AAAACAAAAATTTAAAACTCGAGGGGGCGTAGTTCTGCCG |
| 31 | CTCTCTCTCCCTCTCTTTCCCTCTC | OBD168-p1547 | CAAGTCTTTGATCTTCACTCGAATCTGAGAGACAGAGAGA |
| 32 | GACAGTGCTTTCTCTAAGGCTGGGAACG | OBD168-p745 | TCCTCCGTGGGTCAGGAAGGGTCGACAATCAT |
| 33 | TCGGGACAGAGTGGAGTCTTCCCC | OBD168-p1405 | AGCTCAGTCATGTGAACCTCGACAGTAGGTATTTTTGGAA |
| 34 | GCCCTCTTCCCTGCTGCCCCTGTG | OBD168-p1017 | AGGAGGATCACTTGAGGCCAAGAATTCGATTCGTCC |
| 35 | GAGTCAGGCAGGACCAAGACCACCCAGC | OBD168-p1081 | ACCTTGAGCTAGATACAGAGTGTCGAAATCAGCTTGAGG |
| 36 | CATTCTGCTGTTGATGGGCATTGGGTTG | OBD168-p525 | AGGAGGCATCCGTGTCGAATTTTCATGGCAGCC |
| 37 | GTGGAATGGACCTGCCTGCCTGGC | OBD168-p521 | TTTAGCGTGAGGCGCTCCAGGTCGAGTGGTT |
| 38 | CTCTCTCTCCCTCTCTTTCCCTCTC | OBD168-p1546 | CAAGTCTTTGATCTTCACTCGAATCTGAGAGACAGAGAGA |
| 39 | GCTTGGGCGAGATTCCAGCCTACACGGG | OBD168-p877 | AGAGCTTAAGTTCGAGCAGTGTGGAACCTTCGGG |
| 40 | GGCATTTCGCCACGTTGGCCA | OBD168-p2017 | GACTTGAGCTTCAGGAGTTCGAGACCAGTTTGGCCAACAA |
| 41 | CTCCCCACACTGCCAAGCCCTTACC | OBD168-p1009 | TTGCAGGTCGAAGTGCTCTGTGGTGATGCAGC |
| 42 | AGAAAATATCCACAGAGTTCCAA | OBD168-p1517 | AGGTGCTATGCTTATGACTCGAGATCTCCACTCAGATACC |
| 43 | CTTGCTGGGGGCTTGACTTCTCT | OBD168-p1661 | GGGTACGAGGGGGAGACCTCGATGAGCAGCATGTTATTTA |
| 44 | CATCACCCCAGCCTTTGCTATCACGGGC | OBD168-p965 | AAGTCCCAGGCTTTCGTCTCACGTTCGACAGC |
| 45 | CCACCTGCCTCGGCCTCCCAAA | OBD168-p1417 | CATAAGACTAAGGTTCTGTCGAACCACCTTTATTTACTGT |
| 46 | GCTCCCCACACTGCCAAGCCCTTACC | OBD168-p777 | TTCAACTCGAAGTGCTCTGTGGTGATGCAGCAGG |
| 47 | GGGAGTGGCAGGTCCAAGACTACCC | OBD168-p785 | AGCTCAGGAGTTCGAAACCCAACATAAATGCAAATA |
| 48 | GAGCCTCTAATAAGTGTGGAATCCTGGG | OBD168-p1189 | AGGTGGATCACTTGAGGCCACGAGTTCGAGTAC |
| 49 | TGAGATGGAGTTTAGCTTTTGTTGCC | OBD168-p1329 | TCACCTAAGCTCAGGAGTTCGAGACTAGCTTGGCCAACAT |
| 50 | GTGCCATGGATTCTGTCCTTCGGA | OBD168-p1701 | GCCCATTGGGTACCTAGCTCGAATGCAGGCTGTTGTTGGA |

TABLE 6.a9

| | qPCR Probe | Sequence |
|---|---|---|
| 1 | OBD168-p1531 | CATAAATGCAAGTCTTTTTCGAACAAACACACCTTCAAAC |
| 2 | OBD168-p683 | TGGTTGTTATTCGAACGTGAGACGAAAGCCTGGGAC |
| 3 | OBD168-p923 | TCTATCGACTTTCTCCAAGCATGGATTGCCTGAGCTC |
| 4 | OBD168-p895 | TGGCTCGAATGTTTTCCCAGCTATCTGCCACTCAC |
| 5 | OBD168-p1275 | AGAGTCGAAGACATTTTCTGTTATGTTCTAGACTGAACT |
| 6 | OBD168-p619 | TGGAACTCGAGCTAGGTACCCAATGGGCTACC |
| 7 | OBD168-p635 | GCCATCGGCCAAAATGGGGAAATCGACTTTCTCCA |
| 8 | OBD168-p1631 | TGTGGTTCTTTGAAAAGATCGAATTACCTCTCAAGGCCCC |
| 9 | OBD168-p1145 | AGGGGTGTGTCGAAGTGCTCTGTGGTGATGCA |
| 10 | OBD168-p887 | AGAGGATTCAACTCCCACTGCCTCGACTGCAC |
| 11 | OBD168-p1743 | GTTCTTTTCTTTTCTTTTTCGAACTCCTGAGCTCAAGCAA |
| 12 | OBD168-p519 | AGTTATCGAGCCCTCCGCCAAACCCAACGATTC |
| 13 | OBD168-p1527 | ATTTCTArCTATTATCTATCGAACTCCTGGCCTCGTGATC |
| 14 | OBD168-p791 | TATGGGACACACGGCAGCTCGACCTCGTTTGT |
| 15 | OBD168-p1655 | TCATTGCCACAGTTCACCTCGATTGGTGCATTCACAAACC |
| 16 | OBD168-p691 | ACGTTCGAGAGCTACATCAACAACCTTAGGCGGC |
| 17 | OBD168-p459 | GGGGTTATCGAATGTTTTCCCAGCTATCTGCCACTCA |
| 18 | OBD168-p795 | TCTTTCGACACGGATGCCTCCTAGCTTGATTTGAAC |
| 19 | OBD168-p2035 | CACAGATATATGTTGGTGTCGAGGTCTCCCCCTCGTACCC |
| 20 | OBD168-p1963 | ACAACCATGTCATCAGAATCGATTCATTGATGGTGCATTC |
| 21 | OBD168-p1827 | GAAATGTTTCTTTCTTTTTCGAGGTTCACATGACTGAGCT |
| 22 | OBD168-p1319 | AGACTTTATTTGGACTGTTCGATTGGTGCATTCACAAACC |
| 23 | OBD168-p1383 | CCAGTAACAGTGGACTATTCGACGTCTGCATCCCTAAGCG |
| 24 | OBD168-p1079 | TCTTTGTGGTTAAGTAAACAGATATCGAACTCCTGAGCT |
| 25 | OBD168-p1323 | CTATGGTTAAGTAAAATATCGATTGGTGCATTCACAAACC |
| 26 | OBD168-p1215 | TGCATTCGATCCCAGCTCTCTCTTGTTGGGCAG |
| 27 | OBD168-p591 | ACACTCTCGAAGTTATCATCACCTTGGAGGATGTATAG |
| 28 | OBD168-p871 | TGCAGCATCGAAGTAGTCTTGGTACCCGTGCC |
| 29 | OBD168-p1719 | ATGGAGCATCACTTTGACTCGAACTCTTGACCTCAAGTGA |
| 30 | OBD168-p1911 | CGGCAGAACTACGCCCCCTCGAGTTTTAAATTTTTGTTTT |
| 31 | OBD168-p1549 | TCTCTCTGTCTCTCAGATTCGAGTGAAGATCAAAGACTTG |
| 32 | OBD168-p747 | ATGATTGTCGACCCTTCCTGACCCACGGAGGA |
| 33 | OBD168-p1407 | TTCCAAAAATACCTACTGTCGAGGTTCACATGACTGAGCT |
| 34 | OBD168-p1019 | ACGAATCGAATTCTTGGCCTCAAGTGATCCTCCTGC |
| 35 | OBD168-p1083 | AGTCCCCAAGACCTCAAGCTGATTTCGACACTCTG |
| 36 | OBD168-p527 | AGGGCTGCCATGAAAATTCGACACGGATGCCTC |
| 37 | OBD168-p523 | AACCACTCGACCTGGAGCGCCTCACGCTAAA |
| 38 | OBD168-p1548 | TCTCTCTGTCTCTCAGATTCGAGTGAAGATCAAAGACTTG |
| 39 | OBD168-p879 | TCCCGAAGGTTCCACACTGCTCGAACTTAAGCTC |
| 40 | OBD168-p2019 | TTGTTGGCCAAACTGGTCTCGAACTCCTGAAGCTCAAGTC |
| 41 | OBD168-p1011 | TGCTGCATCACCACAGAGCACTTCGACCTGCA |
| 42 | OBD168-p1519 | GGTATCTGAGTGGAGATCTCGAGTCATAAGCATAGCACCT |
| 43 | OBD168-p1663 | TAAATAACATGCTGCTCATCGAGGTCTCCCCCTCGTACCC |
| 44 | OBD168-p967 | TAGGCTGTCGAACGTGAGACGAAAGCCTGGGA |
| 45 | OBD168-p1419 | ACAGTAAATAAAGGTGGTTCGACAGAACCTTAGTCTTATG |
| 46 | OBD168-p779 | ACCTGCTGCATCACCACAGAGCACTTCGAGTTGAA |
| 47 | OBD168-p787 | ATTTGCATTTATGTTGGGTTTCGAACTCCTGAGCTC |
| 48 | OBD168-p1191 | AGTACTCGAACTCGTGGCCTCAAGTGATCCAC |
| 49 | OBD168-p1331 | ATGTTGGCCAAGCTAGTCTCGAACTCCTGAGCTTAGGTGA |
| 50 | OBD168-p1703 | TCCAACAACAGCCTGCATTCGAGCTAGGTACCCAATGGGC |

TABLE 6.a10

| | probe | PCR Marker Set | qPCR Marker Set |
|---|---|---|---|
| 1 | ORF1_19_56561558_56564720_56759775_56765600_RF | OBD168-1529.1531 | OBD168-q1529.q1531.p1529/p1531 |
| 2 | ORF1_15_69599001_69607474_69822688_69824653_FF | OBD168-681.683 | OBD168-q681.q683.p681/p683 |
| 3 | ORF1_1_16764579_16768424_17044824_17050263_RR | OBD168-921.923 | OBD168-q921.q923.p921/p923 |
| 4 | ORF1_4_24971631_24973362_25125726_25129421_RR | OBD168-893.895 | OBD168-q893.q895.p893/p895 |
| 5 | ORF1_13_57155089_57159703_57172839_57181375_RR | OBD168-1273.1275 | OBD168-q1273.q1275.p1273/p1275 |
| 6 | ORF1_9_98247819_98251791_98368594_98373451_FF | OBD168-617.619 | OBD168-q617.q619.p617/p619 |
| 7 | ORF1_1_234729070_234732599_234814122_234817959_RF | OBD168-633.635 | OBD168-q633.q635.p633/p635 |
| 8 | ORF1_5_54687073_54701690_54895820_54899919_RF | OBD168-1629.1631 | OBD168-q1629.q1631.p1629/p1631 |
| 9 | ORF1_5_77219829_77221017_77232748_77239462_FR | OBD168-1145.1147 | OBD168-q1145.q1147.p1147/p1145 |
| 10 | ORF1_8_57997832_57999789_58156560_58160085_FF | OBD168-885.887 | OBD168-q885.q887.p885/p887 |
| 11 | ORF1_1_224094766_224099331_224328317_224334418_FF | OBD168-1741.1743 | OBD168-q1741.q1743.p1741/p1743 |
| 12 | ORF1_5_141346067_141348820_141380617_141383700_RR | OBD168-517.519 | OBD168-q517.q519.p517/p519 |
| 13 | ORF1_19_54789678_54792948_54890303_54892564_RF | OBD168-1525.1527 | OBD168-q1525.q1527.p1525/p1527 |
| 14 | ORF1_6_14206770_14210218_14395326_14400003_RR | OBD168-789.791 | OBD168-q789.q791.p789/p791 |
| 15 | ORF1_6_13871589_13875376_13978828_13980695_RR | OBD168-1653.1655 | OBD168-q1653.q1655.p1653/p1655 |
| 16 | ORF1_12_52899110_52901976_53003793_53005352_FF | OBD168-689.691 | OBD168-q689.q691.p689/p691 |
| 17 | ORF1_4_24971631_24973362_25161538_25167741_RR | OBD168-457.459 | OBD168-q457.q459.p457/p459 |
| 18 | ORF1_6_33937395_33940607_33982531_33984798_RF | OBD168-793.795 | OBD168-q793.q795.p793/p795 |
| 19 | ORF1_6_34087216_34091346_34298424_34301201_FR | OBD168-2033.2035 | OBD168-q2033.q2035.p2033/p2035 |
| 20 | ORF1_22_29467180_29469328_29549133_29556322_FR | OBD168-1961.1963 | OBD168-q1961.q1963.p1961/p1963 |
| 21 | ORF1_13_112592963_112600705_112765663_112769601_RF | OBD168-1825.1827 | OBD168-q1825.q1827.p1825/p1827 |
| 22 | ORF1_1_89127656_89129856_89152351_89155427_RR | OBD168-1317.1319 | OBD168-q1317.q1319.p1317/p1319 |
| 23 | ORF1_12_62435376_62437096_62567854_62572530_RR | OBD168-1381.1383 | OBD168-q1381.q1383.p1381/p1383 |
| 24 | ORF1_1_117001584_117004481_117080679_117085351_RF | OBD168-1077.1079 | OBD168-q1077.q1079.p1077/p1079 |
| 25 | ORF1_1_89127656_89129856_89172214_89177508_RR | OBD168-1321.1323 | OBD168-q1321.q1323.p1321/p1323 |
| 26 | ORF1_20_4141331_4145409_4240264_4242807_RF | OBD168-1213.1215 | OBD168-q1213.q1215.p1213/p1215 |
| 27 | ORF1_1_234593513_234598020_234817959_234822547_FR | OBD168-589.591 | OBD168-q589.q591.p589/p591 |
| 28 | ORF1_16_86891168_86894116_87066023_87067839_FF | OBD168-869.871 | OBD168-q869.q871.p869/p871 |
| 29 | ORF1_X_44917246_44925908_44940640_44941996_RR | OBD168-1717.1719 | OBD168-q1717.q1719.p1717/p1719 |
| 30 | ORF1_2_54954222_54956881_55045882_55048981_FF | OBD168-1909.1911 | OBD168-q1909.q1911.p1909/p1911 |
| 31 | ORF1_2_92108138_92110573_92242402_92244832_FF | OBD168-1547.1549 | OBD168-q1547.q1549.p1547/p1549 |
| 32 | ORF1_16_29013007_29015111_29280587_29285290_RF | OBD168-745.747 | OBD168-q745.q747.p745/p747 |
| 33 | ORF1_13_112765663_112769601_112883502_112886179_FR | OBD168-1405.1407 | OBD168-q1405.q1407.p1405/p1407 |
| 34 | ORF1_2_28424743_28426988_28597316_28599022_FF | OBD168-1017.1019 | OBD168-q1017.q1019.p1017/p1019 |
| 35 | ORF1_6_14018522_14021640_14102931_14109639_RR | OBD168-1081.1083 | OBD168-q1081.q1083.p1081/p1083 |
| 36 | ORF1_6_33846691_33848820_33982531_33984798_RF | OBD168-525.527 | OBD168-q525.q527.p525/p527 |
| 37 | ORF1_14_74374067_74376797_74492554_74493993_RF | OBD168-521.523 | OBD168-q521.q523.p521/p523 |
| 38 | ORF1_2_92108138_92110573_92210006_92223089_FF | OBD168-1546.1548 | OBD168-q1546.q1548.p1546/p1548 |
| 39 | ORF1_1_33081752_33087352_33257774_33262528_RR | OBD168-877.879 | OBD168-q877.q879.p877/p879 |
| 40 | ORF1_5_77940215_77942645_77971521_77974256_FF | OBD168-2017.2019 | OBD168-q2017.q2019.p2017/p2019 |
| 41 | ORF1_5_77232748_77239462_77325709_77329000_RF | OBD168-1009.1011 | OBD168-q1009.q1011.p1009/p1011 |
| 42 | ORF1_19_54627987_54632181_54748746_54753675_FF | OBD168-1517.1519 | OBD168-q1517.q1519.p1517/p1519 |
| 43 | ORF1_6_33982531_33984798_34087216_34091346_RF | OBD168-1661.1663 | OBD168-q1661.q1663.p1661/p1663 |
| 44 | ORF1_15_69554282_69555739_69599001_69607474_RF | OBD168-965.967 | OBD168-q965.q967.p965/p967 |
| 45 | ORF1_16_70112537_70113568_70359934_70360962_FR | OBD168-1417.1419 | OBD168-q1417.q1419.p1417/p1419 |
| 46 | ORF1_5_76987562_76988733_77232748_77239462_RR | OBD168-777.779 | OBD168-q777.q779.p777/p779 |
| 47 | ORF1_2_8316756_8318269_8515734_8517409_RF | OBD168-785.787 | OBD168-q785.q787.p785/p787 |
| 48 | ORF1_8_8984061_8987615_9203064_9212772_RR | OBD168-1189.1191 | OBD168-q1189.q1191.p1189/p1191 |
| 49 | ORF1_1_165695181_165697826_165952803_165953958_FF | OBD168-1329.1331 | OBD168-q1329.q1331.p1329/p1331 |
| 50 | ORF1_9_98247819_98251791_98381297_98383610_FR | OBD168-1701.1703 | OBD168-q1701.q1703.p1701/p1703 |

TABLE 6.b1

| | | | |
|---|---|---|---|
| 51 | ORF1_1_40249894_40253001_40397735_40401055_FR | 266 | 3261.962157 |
| 52 | ORF1_3_128892194_128893890_129064130_129068087_RF | 179 | 2643.521301 |
| 53 | ORF1_4_113710897_113723443_113810658_113830867_RF | 166 | 2550.222683 |
| 54 | ORF1_19_54627987_54632181_54828537_54832427_FR | 173 | 2600.984915 |
| 55 | ORF1_12_14189_15286_93751_97454_FF | 230 | 3025.599211 |
| 56 | ORF1_13_27789091_27791217_27935736_27938158_RF | 175 | 2611.734713 |
| 57 | ORF1_19_6507876_6510412_6736179_6739196_RR | 186 | 2684.363335 |
| 58 | ORF1_5_71466175_71468826_71713282_71719473_RF | 190 | 2707.443468 |
| 59 | ORF1_22_50201186_50205111_50373464_50374527_FF | 198 | 2781.252576 |
| 60 | ORF1_2_233813067_233815110_233853895_233857139_FR | 200 | 2795.051917 |
| 61 | ORF1_4_168182554_168187989_168317980_168319647_FF | 184 | 2678.622211 |
| 62 | ORF1_2_101327421_101335518_101390331_101393544_FF | 187 | 2686.831942 |
| 63 | ORF1_1_221106643_221120931_221185158_221187933_RF | 309 | 3457.695551 |
| 64 | ORF1_15_31334951_31340691_31546136_31558743_FF | 193 | 2745.03243 |
| 65 | ORF1_7_43795674_43802582_44019092_44021471_RF | 195 | 2772.676516 |
| 66 | ORF1_13_99494037_99497802_99735907_99738338_FF | 197 | 2781.085401 |
| 67 | ORF1_12_14189_15286_200475_202854_FF | 242 | 3107.068033 |
| 68 | ORF1_10_3653748_3655479_3866568_3872650_RR | 209 | 2880.834449 |
| 69 | ORF1_2_74446225_74448326_74574097_74579710_FR | 222 | 2938.850856 |
| 70 | ORF1_6_154995123_154998831_155125636_155128798_RF | 220 | 2915.081526 |
| 71 | ORF1_19_9082207_9087550_9202787_9208911_RR | 221 | 2935.649237 |
| 72 | ORF1_13_27935736_27938158_27961367_27964197_FR | 228 | 2992.736336 |
| 73 | ORF1_6_158943910_158945125_159080063_159084244_RR | 227 | 2988.023125 |
| 74 | ORF1_22_44263290_44267587_44326770_44330737_FF | 204 | 2827.501658 |
| 75 | ORF1_3_32385454_32386698_32541292_32554994_RF | 226 | 2979.923877 |
| 76 | ORF1_19_20562776_20567807_20680600_20685168_RR | 238 | 3074.794125 |
| 77 | ORF1_9_127592507_127594232_127769656_127771134_FR | 233 | 3053.776723 |
| 78 | ORF1_12_134194_137994_14189_15286_RF | 279 | 3316.092388 |
| 79 | ORF1_3_167588699_167598102_167735969_167742420_FF | 236 | 3070.236279 |
| 80 | ORF1_20_2111595_2115703_2369884_2374210_FR | 251 | 3175.82756 |
| 81 | ORF1_2_53649025_53656227_53766349_53775695_FR | 243 | 3112.832366 |
| 82 | ORF1_16_70112537_70113568_70281463_70283063_FF | 262 | 3242.712372 |
| 83 | ORF1_1_35943227_35949162_35991655_35992829_FF | 250 | 3168.607921 |
| 84 | ORF1_11_3589286_3591426_3777994_3782240_FF | 285 | 3336.811355 |
| 85 | ORF1_13_40903938_40912685_40990073_40992867_FR | 256 | 3199.669699 |
| 86 | ORF1_17_46177013_46180826_46195798_46197440_RF | 257 | 3200.455689 |
| 87 | ORF1_11_33667893_33674633_33693896_33698608_RR | 269 | 3274.351845 |
| 88 | ORF1_2_233756599_233760219_233853895_233857139_FR | 281 | 3324.060521 |
| 89 | ORF1_21_44873351_44875256_44901291_44903482_FF | 271 | 3277.035573 |
| 90 | ORF1_1_39712316_39716819_39935069_39938243_RF | 270 | 3274.528209 |
| 91 | ORF1_20_51449039_51452721_51482831_51487722_FF | 278 | 3296.630806 |
| 92 | ORF1_11_10332545_10336410_10435291_10442726_FR | 268 | 3268.369829 |
| 93 | ORF1_17_1305781_1308105_1405058_1407538_FR | 405 | 4046.661111 |
| 94 | ORF1_6_14276298_14278467_14395326_14400003_FR | 298 | 3398.290266 |
| 95 | ORF1_19_54461246_54463256_54564139_54574977_RF | 287 | 3339.709353 |
| 96 | ORF1_2_227722929_227725809_227932124_227939987_FR | 289 | 3350.237008 |
| 97 | ORF1_20_20121190_20124157_20274659_20278486_RR | 282 | 3330.318405 |
| 98 | ORF1_15_90843215_90845291_91002629_91006437_RR | 274 | 3285.787482 |
| 99 | ORF1_22_24255117_24260015_24453446_24457028_FR | 252 | 3177.329191 |
| 100 | ORF1_4_102481630_102483332_102522760_102539034_FR | 369 | 3816.059579 |

TABLE 6.b2

| | | | | |
|---|---|---|---|---|
| 51 | −1.395349802 | 0.100481915 | 0.00018938 | HC |
| 52 | −1.394605873 | 0.07280341 | 9.23358E−05 | HC |
| 53 | −1.394019039 | 0.069334424 | 8.15497E−05 | HC |
| 54 | −1.393940744 | 0.071223807 | 8.73045E−05 | HC |
| 55 | −1.393935973 | 0.090021457 | 0.000146703 | HC |
| 56 | −1.392732078 | 0.07142062 | 8.85578E−05 | HC |
| 57 | −1.392045991 | 0.073868068 | 9.73498E−05 | HC |
| 58 | −1.391817271 | 0.074477563 | 0.000100264 | HC |
| 59 | −1.391435503 | 0.078395267 | 0.000109982 | HC |
| 60 | −1.389447743 | 0.078941759 | 0.000111867 | HC |
| 61 | −1.388162304 | 0.074121954 | 9.6634E−05 | HC |
| 62 | −1.387816432 | 0.073706177 | 9.76587E−05 | HC |
| 63 | −1.387782969 | 0.105280142 | 0.0002305 | HC |
| 64 | −1.385630516 | 0.076882938 | 0.000105136 | HC |
| 65 | −1.385106801 | 0.078761241 | 0.000108821 | HC |
| 66 | −1.383613903 | 0.078776945 | 0.000109959 | HC |
| 67 | −1.382570148 | 0.093653852 | 0.000160585 | HC |
| 68 | −1.381365435 | 0.083791812 | 0.000124083 | HC |
| 69 | −1.378245812 | 0.084452402 | 0.00013284 | HC |
| 70 | −1.377515984 | 0.082886988 | 0.000129204 | HC |
| 71 | −1.376577399 | 0.084519141 | 0.000132347 | HC |
| 72 | −1.376502872 | 0.087492255 | 0.000141342 | HC |
| 73 | −1.376374612 | 0.087406474 | 0.000140584 | HC |
| 74 | −1.376326886 | 0.080520984 | 0.000116387 | HC |
| 75 | −1.375231562 | 0.086983939 | 0.000139288 | HC |
| 76 | −1.374198345 | 0.091906279 | 0.000154984 | HC |
| 77 | −1.37322453 | 0.091712649 | 0.000151409 | HC |
| 78 | −1.372472194 | 0.101278707 | 0.000200211 | HC |
| 79 | −1.371647873 | 0.092218553 | 0.000154204 | HC |
| 80 | −1.370634715 | 0.097260863 | 0.000172973 | HC |
| 81 | −1.370591898 | 0.093857745 | 0.0001616 | HC |
| 82 | −1.369001392 | 0.099993659 | 0.000185626 | HC |
| 83 | −1.367985092 | 0.096899096 | 0.000171643 | HC |
| 84 | −1.367790553 | 0.101252915 | 0.000204464 | HC |
| 85 | −1.366608289 | 0.097809856 | 0.000177414 | HC |
| 86 | −1.366352925 | 0.097510393 | 0.000177562 | HC |
| 87 | −1.365301253 | 0.100643162 | 0.000191824 | HC |
| 88 | −1.365290466 | 0.101375894 | 0.00020184 | HC |
| 89 | −1.365261052 | 0.100177474 | 0.000192356 | HC |
| 90 | −1.363988483 | 0.100288669 | 0.000191858 | HC |
| 91 | −1.36386899 | 0.099642396 | 0.00019627 | HC |
| 92 | −1.363654072 | 0.100396086 | 0.000190641 | HC |
| 93 | −1.363297723 | 0.135705371 | 0.000389419 | HC |
| 94 | −1.363180618 | 0.102982649 | 0.000217443 | HC |
| 95 | −1.362941181 | 0.100842256 | 0.000205064 | HC |
| 96 | −1.362639868 | 0.101213262 | 0.000207253 | HC |
| 97 | −1.362102923 | 0.101659702 | 0.000203125 | HC |
| 98 | −1.361938026 | 0.099977846 | 0.000194097 | HC |
| 99 | −1.36144754 | 0.097030319 | 0.00017325 | HC |
| 100 | −1.361141308 | 0.122607616 | 0.00032056 | HC |

TABLE 6.b3

| | |
|---|---|
| 51 | GGCAGGAGGATTGCTTGAGTTCAGGAGTTCGAACAAGATATTAAGTATTAGAAGAACAAA |
| 52 | CACCACCAGAGTAGCTAGATACAGAGTGTCGATTGTCATGTTTTCACTGACAAATATGTC |
| 53 | GGCAGGCAGATCACCTGAGGTTTGGAGTTCGAATGTTTCTCTAATGCATGTCTTCTTTGG |
| 54 | GTGGTGGTACAGGTGCTATGCTTATGACTCGAGACATGTGGAGTCACCCCATTTGCAGTG |
| 55 | GTGTTTCCTAGTTAGCTCAGAGCCTCAGTCGAAGGAAGCTTAGGAATATAGCTTATGTGT |
| 56 | AGCAGGCAGATCACTTGAGGTCAGAAATTCGACCTCCTTGCTCAACTGTCTTTTATCCAC |
| 57 | AGGAGCTGGGGTGTATGTCAACAATTTCTCGAACTCCCAGGTTCAAACGATCCTCTCACC |
| 58 | AAGGAGCTGGTCGGTATTCCCCTCGCTCTCGAGTAAAAGTGGCAGAAGATGGTTCCATTA |
| 59 | AGGGAGTAGAGGAATCGGGAGCCAGTGTTCGAATAATACATCTCTTCAATATTCCTGATT |
| 60 | AGTAATGTCCTTTAGAGCTGTTTTCTCCTCGAAGGGCTGGTTGTACTGCGGAGGAGGAAG |
| 61 | AAGGCAGGCAGATCACTAGGTCAGGAGTTCGAAACCCACACTGCAAGTATCAACAGGCAA |
| 62 | GAGGCAGGCGGATCACGAGGTCAATAGATCGATAGGAAACAAACATTTGATCCGTTAGTA |
| 63 | AGTGGCATGATCATAGTTCACTGCAGCCTCGATACACATTTTCAAAGTGCTTTTCAAAAA |
| 64 | GGCAGGAGAATTGCTTGAGCCCAGAAGTTCGAGTTTTCCCCTTGCTCCTAAATCCCAGTC |
| 65 | GGCAGGCAGATCACTTGAGGTAAGGAGTTCGAAAATTAGCAGAAAAAAGCAAGTAAAGAT |
| 66 | GGCAGGCGGATCACCTGAGTTCAGGAGTTCGAGATGACAGCTAAACATAAAATGAACCAA |
| 67 | GTGTTTCCTAGTTAGCTCAGAGCCTCAGTCGAGTCATATTTGAATGTCTACTACATCATC |
| 68 | AGGCAGGCAGATCACTTGAGGTTAGGGTTCGACCAGCAGCAAAGACAGCAAGTATTCCAG |
| 69 | GCAGTGAGCCGAGATCATGCCATTATACTCGAAACAGGAAAAAAAGAAGTCTGCTTCTGA |
| 70 | GGCAGGAGGATCACTTGAGCCTAGGAAGTCGATGTTTCTTGAATTGTTTTATTTGTGTTG |
| 71 | AGGCAAGAGAATCACTTGAACCCGGGAGTCGAGATCAGCCTGACCAACATGAAACCCCGT |
| 72 | AGCAGGCAGATCACTTGAGGTCAGAAATTCGAAACGAAGGGCTAAGCCCCCCTCGCCCGG |
| 73 | AGCAGGAGGATCACTTGAGGCCAGGGATTCGATCCTCAAGCTAAGAATAAAAACACCAAG |
| 74 | CAAAGGCAAATGCGTCCTGTCACTCTCCTCGAGGTTATATTTATTCTAATTATTATTATC |
| 75 | GTTAAAGTGATTCTCCTGCCTCAGACTCTCGATACATAAGGACTTAGGAAAAATGATAAA |
| 76 | GAGGGAGGGAGATAAGCTTAAGAAGAGTTCGAGACACTTATTTTGGAGATGGCATCTTGC |
| 77 | GGCAGGTGGATCACCTGAGGTCAGGAATTCGAAAAAAAAAAAAAAAAGAAATAAAGAAAA |
| 78 | GTGTTTCCTAGTTAGCTCAGAGCCTCAGTCGAATACACACTCATTGAAGGCCCACTATGT |
| 79 | GAGGTGGGCAGATCACGAGGACAGGAGATCGAGCCTGTAAACAAAAAGCAAACAACTACT |
| 80 | CCCACACAATTTAAATCTGTCTTGTACATCGAGGTGAGAGTGAGCTGAGATGGCGCCACT |
| 81 | AGCAGGCAGATCATCTGAGGTTGGGAGTTCGAAGACACAATTTCTTGTTAGTGACTCTAT |
| 82 | TAAAGCTATGCATAAGACTAAGGTTCTGTCGATACATATTTGTTTAATGAACAAACATCC |
| 83 | AGGTTTCTCCATGTTGGTCAGTCTGGTCTCGAAGTCACTCCTAACTCATAGAAAATGTGT |
| 84 | AGGCAGCTTGTCTAGGTAAAGTTCCGGGTCGAATAGGCCTAAGTTTGTGGAATGATAGAT |
| 85 | AAGGCAGGTGGATCACGAGGTCAGCAGATCGATACATAATTTTACAAAACCAAACACATT |
| 86 | GGCAGGCAGATCACCTGAGGCCAGGAATTCGAAATACTATGCTAAGATTCAACATTACTC |
| 87 | CTGGGGGTACCGTGATCGGGGGTAGGCCTCGAAAGGTCATTATAAGGATGGAGTGAGATG |
| 88 | TGACACAGTCAAACATTAACTTGGTGTATCGAAGGGCTGGTTGTACTGCGGAGGAGGAAG |
| 89 | TTTGCAGGTGTGTGGTTAACACTATCCTTCGACCTGTACTATCTGATGGACCTCTCCTAC |
| 90 | AGTGGTGCCATCATAGCTCACTGCAGCCTCGAATAAAAGAAAATAAAAAGACATGATTAA |
| 91 | CCAAGTGCACCACGTGAACAAAACAATCTCGATTGGGAATAACTCACCTTTTTTATGCCA |
| 92 | AGGCAGGTGGATCACTTGAGCTCAGGATTCGAAAACATTAACTGAAAGAAATTGGACACA |
| 93 | TCAGACAGATCAGGAAAACGGAATGACATCGAGAAAGTGGAACTGGTAAGCATAGGGGTC |
| 94 | CTACCCAGCCCCTGGTGGGCAGGTGAGTTCGAGCTGCCGTGTGTCCCATAGCCACCAGCT |
| 95 | GTGGTCGTACAGGTGCTATGCTTATGACTCGAGCTTGCACTCCGGTACCCGACCCGGCGC |
| 96 | GAGGCTGAGGCAAGAGAATTGCTTGAACTCGATTCAGCATATGAAGGGGAAGTGATACAG |
| 97 | GGTGGGAGGATTGCTTGAGCCTGAAAGGTCGAAAAATAAAAATGAAAATAAAAAAAAGGC |
| 98 | GGCAGGAGGATCGCTTGAACCCAGGAGTTCGAAATAAAAAAAAAAAAAAAAAGAAAAGAA |
| 99 | CTCCAGCCGCAAGAAGTACAGCAGCACCTCGATCTCACTAGTAATTAGGAAAATGAAAAT |
| 100 | CAGCAGTGCCAGCCTACCGGTGCTGCACTCGACAGAAAGCTTAAGGGCAAAGATGGTTTT |

TABLE 6.b4

| | | | | | | | | | | |
|---|---|---|---|---|---|---|---|---|---|---|
| 51 | 1 | 40252970 | 40252999 | 40397737 | 40397766 | 1 | 40249000 | 40252999 | 40397737 | 40401736 |
| 52 | 3 | 128892196 | 128892225 | 129068056 | 129068085 | 3 | 128892196 | 128896195 | 129064086 | 129068085 |
| 53 | 4 | 113710899 | 113710928 | 113830836 | 113830865 | 4 | 113710899 | 113714898 | 113826866 | 113830865 |
| 54 | 19 | 54632150 | 54632179 | 54828539 | 54828568 | 19 | 54628180 | 54632179 | 54828539 | 54832538 |
| 55 | 12 | 15255 | 15284 | 97423 | 97452 | 12 | 11285 | 15284 | 93453 | 97452 |
| 56 | 13 | 27789093 | 27789122 | 27938127 | 27938156 | 13 | 27789093 | 27793092 | 27934157 | 27938156 |
| 57 | 19 | 6507878 | 6507907 | 6736181 | 6736210 | 19 | 6507878 | 6511877 | 6736181 | 6740180 |
| 58 | 5 | 71466177 | 71466206 | 71719442 | 71719471 | 5 | 71466177 | 71470176 | 71715472 | 71719471 |
| 59 | 22 | 50205080 | 50205109 | 50374496 | 50374525 | 22 | 50201110 | 50205109 | 50370526 | 50374525 |
| 60 | 2 | 233815079 | 233815108 | 233853897 | 233853926 | 2 | 233811109 | 233815108 | 233853897 | 233857896 |
| 61 | 4 | 168187958 | 168187987 | 168319616 | 168319645 | 4 | 168183988 | 168187987 | 168315646 | 168319645 |
| 62 | 2 | 101335487 | 101335516 | 101393513 | 101393542 | 2 | 101331517 | 101335516 | 101389543 | 101393542 |
| 63 | 1 | 221106645 | 221106674 | 221187902 | 221187931 | 1 | 221106645 | 221110644 | 221183932 | 221187931 |
| 64 | 15 | 31340660 | 31340689 | 31558712 | 31558741 | 15 | 31336690 | 31340689 | 31554742 | 31558741 |
| 65 | 7 | 43795676 | 43795705 | 44021440 | 44021469 | 7 | 43795676 | 43799675 | 44017470 | 44021469 |
| 66 | 13 | 99497771 | 99497800 | 99738307 | 99738336 | 13 | 99493801 | 99497800 | 99734337 | 99738336 |
| 67 | 12 | 15255 | 15284 | 202823 | 202852 | 12 | 11285 | 15284 | 198853 | 202852 |
| 68 | 10 | 3653750 | 3653779 | 3866570 | 3866599 | 10 | 3653750 | 3657749 | 3866570 | 3870569 |
| 69 | 2 | 74448295 | 74448324 | 74574099 | 74574128 | 2 | 74444325 | 74448324 | 74574099 | 74578098 |
| 70 | 6 | 154995125 | 154995154 | 155128767 | 155128796 | 6 | 154995125 | 154999124 | 155124797 | 155128796 |
| 71 | 19 | 9082209 | 9082238 | 9202789 | 9202818 | 19 | 9082209 | 9086208 | 9202789 | 9206788 |
| 72 | 13 | 27938127 | 27938156 | 27961369 | 27961398 | 13 | 27934157 | 27938156 | 27961369 | 27965368 |
| 73 | 6 | 158943912 | 158943941 | 159080065 | 159080094 | 6 | 158943912 | 158947911 | 159080065 | 159084064 |
| 74 | 22 | 44267556 | 44267585 | 44330706 | 44330735 | 22 | 44263586 | 44267585 | 44326736 | 44330735 |
| 75 | 3 | 32385456 | 32385485 | 32554963 | 32554992 | 3 | 32385456 | 32389455 | 32550993 | 32554992 |
| 76 | 19 | 20562778 | 20562807 | 20680602 | 20680631 | 19 | 20562778 | 20566777 | 20680602 | 20684601 |
| 77 | 9 | 127594201 | 127594230 | 127769658 | 127769687 | 9 | 127590231 | 127594230 | 127769658 | 127773657 |
| 78 | 12 | 134196 | 134225 | 15255 | 15284 | 12 | 134196 | 138195 | 11285 | 15284 |
| 79 | 3 | 167598071 | 167598100 | 167742389 | 167742418 | 3 | 167594101 | 167598100 | 167738419 | 167742418 |
| 80 | 20 | 2115672 | 2115701 | 2369886 | 2369915 | 20 | 2111702 | 2115701 | 2369886 | 2373885 |
| 81 | 2 | 53656196 | 53656225 | 53766351 | 53766380 | 2 | 53652226 | 53656225 | 53766351 | 53770350 |
| 82 | 16 | 70113537 | 70113566 | 70283032 | 70283061 | 16 | 70109567 | 70113566 | 70279062 | 70283061 |
| 83 | 1 | 35949131 | 35949160 | 35992798 | 35992827 | 1 | 35945161 | 35949160 | 35988828 | 35992827 |
| 84 | 11 | 3591395 | 3591424 | 3782209 | 3782238 | 11 | 3587425 | 3591424 | 3778239 | 3782238 |
| 85 | 13 | 40912654 | 40912683 | 40990075 | 40990104 | 13 | 40908684 | 40912683 | 40990075 | 40994074 |
| 86 | 17 | 46177015 | 46177044 | 46197409 | 46197438 | 17 | 46177015 | 46181014 | 46193439 | 46197438 |
| 87 | 11 | 33667895 | 33667924 | 33693898 | 33693927 | 11 | 33667895 | 33671894 | 33693898 | 33697897 |
| 88 | 2 | 233760188 | 233760217 | 233853897 | 233853926 | 2 | 233756218 | 233760217 | 233853897 | 233857896 |
| 89 | 21 | 44875225 | 44875254 | 44903451 | 44903480 | 21 | 44871255 | 44875254 | 44899481 | 44903480 |
| 90 | 1 | 39712318 | 39712347 | 39938212 | 39938241 | 1 | 39712318 | 39716317 | 39934242 | 39938241 |
| 91 | 20 | 51452690 | 51452719 | 51487691 | 51487720 | 20 | 51448720 | 51452719 | 51483721 | 51487720 |
| 92 | 11 | 10336379 | 10336408 | 10435293 | 10435322 | 11 | 10332409 | 10336408 | 10435293 | 10439292 |
| 93 | 17 | 1308074 | 1308103 | 1405060 | 1405089 | 17 | 1304104 | 1308103 | 1405060 | 1409059 |
| 94 | 6 | 14278436 | 14278465 | 14395328 | 14395357 | 6 | 14274466 | 14278465 | 14395328 | 14399327 |
| 95 | 19 | 54461248 | 54461277 | 54574946 | 54574975 | 19 | 54461248 | 54465247 | 54570976 | 54574975 |
| 96 | 2 | 227725778 | 227725807 | 227932126 | 227932155 | 2 | 227721808 | 227725807 | 227932126 | 227936125 |
| 97 | 20 | 20121192 | 20121221 | 20274661 | 20274690 | 20 | 20121192 | 20125191 | 20274661 | 20278660 |
| 98 | 15 | 90843217 | 90843246 | 91002631 | 91002660 | 15 | 90843217 | 90847216 | 91002631 | 91006630 |
| 99 | 22 | 24259984 | 24260013 | 24453448 | 24453477 | 22 | 24256014 | 24260013 | 24453448 | 24457447 |
| 100 | 4 | 102483301 | 102483330 | 102522762 | 102522791 | 4 | 102479331 | 102483330 | 102522762 | 102526761 |

TABLE 6.b5

| | | |
|---|---|---|
| 51 | ORF1_1_40249894_40253001_40397735_40401055_FR | OBD168-1737 |
| 52 | ORF1_3_128892194_128893890_129064130_129068087_RF | OBD168-1989 |
| 53 | ORF1_4_113710897_113723443_113810658_113830867_RF | OBD168-2005 |
| 54 | ORF1_19_54627987_54632181_54828537_54832427_FR | OBD168-1521 |
| 55 | ORF1_12_14189_15286_93751_97454_FF | OBD168-1369 |
| 56 | ORF1_13_27789091_27791217_27935736_27938158_RF | OBD168-1137 |
| 57 | ORF1_19_6507876_6510412_6736179_6739196_RR | OBD168-1885 |
| 58 | ORF1_5_71466175_71468826_71713282_71719473_RF | OBD168-953 |
| 59 | ORF1_22_50201186_50205111_50373464_50374527_FF | OBD168-577 |
| 60 | ORF1_2_233813067_233815110_233853895_233857139_FR | OBD168-1937 |
| 61 | ORF1_4_168182554_168187989_168317980_168319647_FF | OBD168-2013 |
| 62 | ORF1_2_101327421_101335518_101390331_101393544_FF | OBD168-549 |
| 63 | ORF1_1_221106643_221120931_221185158_221187933_RF | OBD168-717 |
| 64 | ORF1_15_31334951_31340691_31546136_31558743_FF | OBD168-929 |
| 65 | ORF1_7_43795674_43802582_44019092_44021471_RF | OBD168-2061 |
| 66 | ORF1_13_99494037_99497802_99735907_99738338_FF | OBD168-1401 |
| 67 | ORF1_12_14189_15286_200475_202854_FF | OBD168-1373 |
| 68 | ORF1_10_3653748_3655479_3866568_3872650_RR | OBD168-1745 |
| 69 | ORF1_2_74446225_74448326_74574097_74579710_FR | OBD168-1253 |
| 70 | ORF1_6_154995123_154998831_155125636_155128798_RF | OBD168-1673 |
| 71 | ORF1_19_9082207_9087550_9202787_9208911_RR | OBD168-1241 |
| 72 | ORF1_13_27935736_27938158_27961367_27964197_FR | OBD168-1297 |
| 73 | ORF1_6_158943910_158945125_159080063_159084244_RR | OBD168-2041 |
| 74 | ORF1_22_44263290_44267587_44326770_44330737_FF | OBD168-1969 |
| 75 | ORF1_3_32385454_32386698_32541292_32554994_RF | OBD168-1601 |
| 76 | ORF1_19_20562776_20567807_20680600_20685168_RR | OBD168-1493 |
| 77 | ORF1_9_127592507_127594232_127769656_127771134_FR | OBD168-2097 |
| 78 | ORF1_12_134194_137994_14189_15286_RF | OBD168-1789 |
| 79 | ORF1_3_167588699_167598102_167735969_167742420_FF | OBD168-1225 |
| 80 | ORF1_20_2111595_2115703_2369884_2374210_FR | OBD168-741 |
| 81 | ORF1_2_53649025_53656227_53766349_53775695_FR | OBD168-957 |
| 82 | ORF1_16_70112537_70113568_70281463_70283063_FF | OBD168-1421 |
| 83 | ORF1_1_35943227_35949162_35991655_35992829_FF | OBD168-1309 |
| 84 | ORF1_11_3589286_3591426_3777994_3782240_FF | OBD168-505 |
| 85 | ORF1_13_40903938_40912685_40990073_40992867_FR | OBD168-593 |
| 86 | ORF1_17_46177013_46180826_46195798_46197440_RF | OBD168-1449 |
| 87 | ORF1_11_33667893_33674633_33693896_33698608_RR | OBD168-537 |
| 88 | ORF1_2_233756599_233760219_233853895_233857139_FR | OBD168-1945 |
| 89 | ORF1_21_44873351_44875256_44901291_44903482_FF | OBD168-609 |
| 90 | ORF1_1_39712316_39716819_39935069_39938243_RF | OBD168-1289 |
| 91 | ORF1_20_51449039_51452721_51482831_51487722_FF | OBD168-801 |
| 92 | ORF1_11_10332545_10336410_10435291_10442726_FR | OBD168-1753 |
| 93 | ORF1_17_1305781_1308105_1405058_1407538_FR | OBD168-713 |
| 94 | ORF1_6_14276298_14278467_14395326_14400003_FR | OBD168-1069 |
| 95 | ORF1_19_54461246_54463256_54564139_54574977_RF | OBD168-1513 |
| 96 | ORF1_2_227722929_227725809_227932124_227939987_FR | OBD168-1933 |
| 97 | ORF1_20_20121190_20124157_20274659_20278486_RR | OBD168-1949 |
| 98 | ORF1_15_90843215_90845291_91002629_91006437_RR | OBD168-1000 |
| 99 | ORF1_22_24255117_24260015_24453446_24457028_FR | OBD168-541 |
| 100 | ORF1_4_102481630_102483332_102522760_102539034_FR | OBD168-2001 |

TABLE 6.b6

| | | | |
|---|---|---|---|
| 51 | GCTGCTCAACAAGACACAATGTAAGG | OBD168-1739 | CACCATTCTTTCTACCTGGAATGGCT |
| 52 | CCACCCACATCCTGCTGATTGGTCCA | OBD168-1991 | TACACTTAAGAATTTTTGTATACATG |
| 53 | CTATCGGAGACTCTAAATGTA | OBD168-2007 | ACCTAAAGGCTCTTTAACGTA |
| 54 | GTAAGGAAGGAGAAGATGAACAC | OBD168-1523 | TCTTCCTGTTCTTCCTGGAGGAG |
| 55 | TGGCTCGTGGCTCTCACTGCA | OBD168-1371 | TGCCTTCTGTCATACATTTTG |
| 56 | CACAGCAGGTGGGAAAATGTAGCC | OBD168-1139 | CTGGCACATAGCAGGTGGCTCGT |
| 57 | ACACACAAGCTGACACAGCAA | OBD168-1887 | GGCAACATAGTGAGAACCCCG |
| 58 | ATCTACTCTGCCGTGGATGATGCCTC | OBD168-955 | CAGCCTGACTCAATGACCAGTAAAGA |
| 59 | TAGAGACAAAGTAGAATGGTGGGTCC | OBD168-579 | GCGGATGGATGGCTTGAGTCTGAAAG |
| 60 | CCACGGATAGCTGCAGACATCCAGA | OBD168-1939 | TTGGTGGGGCTGGGGGTGCTCCTGA |
| 61 | AAATAAGTAAAGACGTAAACCAGCA | OBD168-2015 | TATGTAACAATACATGAACTTTGAG |
| 62 | TGGTGAAAACCTATCTCGGCTGGGCA | OBD168-551 | GAAGTGTGATGTGCCTCTCTTTCTGT |
| 63 | CCAGCAGACAAATCCCAGTTCAATGC | OBD168-719 | TCACAAAGCAGGAGGGAGAACAGGTT |
| 64 | AGAAACAGTCCTGCTAACATCACCCC | OBD168-931 | CTTTCATTCAACACCCACGGAGGAGT |
| 65 | CATTCTGCAACAGCCCCACACAGAA | OBD168-2063 | CCCAAAATGCTGGGATTACAGGCGT |
| 66 | AAGAAAAAGAAAAAAAGACACAAAT | OBD168-1403 | ATTCATGTAACAACTTAATTAGGGA |
| 67 | CAGACTGGGGTGAAGAGTTCAGTCAC | OBD168-1375 | AGGGGCTGAAATGATGGATACCTGTG |
| 68 | ACTCCGTCTCAAAAAAAAAGAAAAAA | OBD168-1747 | TAAAATAACTGTATTTAAGGGCTACT |
| 69 | TTGGGAGGCTGAGGCAGGAGAAT | OBD168-1255 | GGGAGTGAGAAAGAGGAAGTATCAAA |
| 70 | GGAGTTCAAGACCAGCCTGGAGC | OBD168-1675 | TTACGGTTACCGCTGCCCCAGCC |
| 71 | TACACAGGTTGGAAAGACGAAGAAAA | OBD168-1243 | GAGTAGTTGGGATTACAGGCGGGT |
| 72 | CTCACGCCTGTAATCCCAGCACT | OBD168-1299 | CCCTTCCAACCCAATCACCCCAC |
| 73 | AAATTACAATATACAAATTACATACA | OBD168-2043 | TGTGCCTGCTGCTATGCCCAAATGCC |
| 74 | CCCCACCACCACCCTCAGCCAAGCC | OBD168-1971 | GGCAGCTAGCAAGAGTGGGGGAGAC |
| 75 | ACTTTAATAACATCTGGTAAT | OBD168-1603 | TCCTTCTCTGTAAAGAGGAAC |
| 76 | GCAAACGGAGAGTGAAAAAGTCAT | OBD168-1495 | AAGTGCCCTATTTTCATATCCCCT |
| 77 | GGAAGAATTTGAGGGTTAAGGAGA | OBD168-2099 | CCTCACTATTTGATCCCAGGCCTG |
| 78 | AACGGGAAAGCCACAGACTGGGGTGA | OBD168-1791 | TAACCCTAGGAGACTCCGTGTACTAC |
| 79 | TTGAAATGTGAGAAGGTTGGGCTGGG | OBD168-1227 | AGAACAAGAAGTCTGAGGAGAGTAG |
| 80 | GCCCAGCCTCTCCTCATCAGTGA | OBD168-743 | GACACCCTCAAGCACACACTCAG |
| 81 | TATTCCGAGACCATTGTGTGTGAGTC | OBD168-959 | AGGGCATTCTCCTAATAACTGTGATG |
| 82 | GATAGTGTGGCCCAAGATGCAA | OBD168-1423 | CGACTCAGGTGAGCTGTTTGAT |
| 83 | GTATGCAATGGCACGATCTCG | OBD168-1311 | AAATGTTTGGATGGTTAAGTC |
| 84 | CATAGTTCTCATAGACCAGGGTTTGC | OBD168-507 | AAGTTGTGCCAAAGCCACCTGTGCCA |
| 85 | CCCTTTTCTCAGCACAGCAGTGGAAT | OBD168-595 | AAAGACCTGATGTATTTCCTACCAGC |
| 86 | ATGTTATCCTCTTATATTCCACCA | OBD168-1451 | GGGAAGGCTCTGTTACTTGTAGAT |
| 87 | TTTTCTGCCAGGCTCTGCTGCGA | OBD168-539 | GAAGAGTTCACAATACACTAAGGGAA |
| 88 | ATAAAGCTCCACCTTCTTTAT | OBD168-1947 | GGGGCTGGGGGTGCTCCTGAG |
| 89 | GGGCTGGAGTTGAGGCTTGGTTT | OBD168-611 | CAGGGTCTGGGAAAGGACTGGGT |
| 90 | AATCCTCCTGCCTCAGCCTCCCG | OBD168-1291 | TTTCTTCTTCCATCCCCATCATTGC |
| 91 | TCCCCACACAGACCTCCTCTCCT | OBD168-803 | GAACCCCAAGATGGTGAAGAGGC |
| 92 | CCCAGGCTTGTCTTCAACTTCTG | OBD168-1755 | CTCCTAGAAACCATTAATCTACT |
| 93 | GGACAAGGAGGCAGAGTGACACG | OBD168-715 | CTGTCTTCACTGCTCCTCTCAGG |
| 94 | GGGCACCCTCTCCTGTTTCACTC | OBD168-1071 | TGGGCAGTGGAAATAAGGCAGGC |
| 95 | GCTTCATTCTGTGTAAGGAAGGAG | OBD168-1515 | CCCCAAACACGGAGGCCCCAAGGG |
| 96 | GCTCACACCTGTAATCCCAGCAC | OBD168-1935 | TCATTTTTAAGCTTCTAAGTAAC |
| 97 | TAATCCCAGCACTTTGGGAGGCTGA | OBD168-1951 | TAATATAATGTTATATTAAATAAAT |
| 98 | TTTTGAAAGAGGCAAGAGAAGTGG | OBD168-998 | TTTATGAAGCACACACGATACCAG |
| 99 | AGGATGCTCTGTCTCTTGCTGTCCAG | OBD168-543 | GTTGGCTGACTGTGAATGGTGGTAAA |
| 100 | ACCAGGGCACTTGCGGGCCAGCTGGA | OBD168-2003 | TTTTCAGTTATAAGAAGAATACTAGT |

TABLE 6.b7

| | | | |
|---|---|---|---|
| 51 | OBD168-q1737 | GGCTATGCTGCTCAACAAGACAC | OBD168-q1739 |
| 52 | OBD168-q1989 | ACATCCTGCTGATTGGTCCATT | OBD168-q1991 |
| 53 | OBD168-q2005 | ATGTAGCCATCATTCTTATGC | OBD168-q2007 |
| 54 | OBD168-q1521 | TGATGGCTTCATTCTGTGTAAGG | OBD168-q1523 |
| 55 | OBD168-q1369 | TGAAGAGATCCGACATCAAGTGCCCA | OBD168-q1371 |
| 56 | OBD168-q1137 | GGAGTTACACGATGCCTGCTTACACCTC | OBD168-q1139 |
| 57 | OBD168-q1885 | CACAAGCTGACACAGCAAGCA | OBD168-q1887 |
| 58 | OBD168-q953 | GCCGCCCTGCTGCTGATGCTACC | OBD168-q955 |
| 59 | OBD168-q577 | CTGGTCACAGAGAGACAAATCCTGGGTG | OBD168-q579 |
| 60 | OBD168-q1937 | CATCTTTTGAACCACGGATAGCTG | OBD168-q1939 |
| 61 | OBD168-q2013 | CCTAGTTTGGGGAATTTTTGAGACTC | OBD168-q2015 |
| 62 | OBD168-q549 | GTGAAAACCTATCTCGGCTGGGCACGGG | OBD168-q551 |
| 63 | OBD168-q717 | GACCAGCAGACAAATCCCAGTTCAATGC | OBD168-q719 |
| 64 | OBD168-q929 | CTGCGGTTGGGTGGCACAGGGCTG | OBD168-q931 |
| 65 | OBD168-q2061 | GAAATCTCATTCTGCAACAGCCCCAC | OBD168-q2063 |
| 66 | OBD168-q1401 | ACAGAGTGAGATTCTGTTTCAAAA | OBD168-q1403 |
| 67 | OBD168-q1373 | CAACGGGAAAGCCACAGACTGGGG | OBD168-q1375 |
| 68 | OBD168-q1745 | AGCTAAACTCCGTCTCAAAAAA | OBD168-q1747 |
| 69 | OBD168-q1253 | TTGGGAGGCTGAGGCAGGTGGAT | OBD168-q1255 |
| 70 | OBD168-q1673 | AGGAGTTCAAGACCAGCCTGGAGCA | OBD168-q1675 |
| 71 | OBD168-q1241 | GTGAACAAACAGACCTCCAGATGTGGC | OBD168-q1243 |
| 72 | OBD168-q1297 | GCGGCTCACGCCTGTAATCCCAGCAC | OBD168-q1299 |
| 73 | OBD168-q2041 | TTATAATGATTTTATAAATGTACT | OBD168-q2043 |
| 74 | OBD168-q1969 | CTCAGCCAAGCCACAGACATGGCTG | OBD168-q1971 |
| 75 | OBD168-q1601 | TTTGAATTTTTTTTTTTTTTTTTTTT | OBD168-q1603 |
| 76 | OBD168-q1493 | AAATCAGCATGTTCTTAGGAGGGA | OBD168-q1495 |
| 77 | OBD168-q2097 | TAGGATGTATTCATAGGGGATG | OBD168-q2099 |
| 78 | OBD168-q1789 | CTCGTGGCTCTCACTGCAACG | OBD168-q1791 |
| 79 | OBD168-q1225 | TTTTGAAATGTGAGAAGGTTGGGCTGGG | OBD168-q1227 |
| 80 | OBD168-q741 | ACCACCCAGAACAAGCCAGCACATTTGC | OBD168-q743 |
| 81 | OBD168-q957 | GTTATTCCGAGACCATTGTGTGTGAGTC | OBD168-q959 |
| 82 | OBD168-q1421 | TGAATCCAACAAACGGGAACTGGAT | OBD168-q1423 |
| 83 | OBD168-q1309 | CTGGGTTCAAGCAATTCTCCT | OBD168-q1311 |
| 84 | OBD168-q505 | ATCATAGTTCTCATAGACCAGGGTTTGC | OBD168-q507 |
| 85 | OBD168-q593 | CATACCCTTTTCTCAGCACAGCAGTGGA | OBD168-q595 |
| 86 | OBD168-q1449 | CCTCATTACCACCATTCCTTGTTAAT | OBD168-q1451 |
| 87 | OBD168-q537 | TCTGCCAGGCTCTGCTGCGAGGC | OBD168-q539 |
| 88 | OBD168-q1945 | ATTAAGAAACCTAATAAAGCTCCAC | OBD168-q1947 |
| 89 | OBD168-q609 | GTTCCGCCTACCCAGGTCCACACC | OBD168-q611 |
| 90 | OBD168-q1289 | TTAGGCTGGTCTTGAACTCCTGGGCTCA | OBD168-q1291 |
| 91 | OBD168-q801 | GCCCAACCTCATCCCCACACAGACCTCC | OBD168-q803 |
| 92 | OBD168-q1753 | TGCTATGTTGCCCAGGCTTGTCTTC | OBD168-q1755 |
| 93 | OBD168-q713 | GCCTCCAGGAAGGTGTGGGTGGC | OBD168-q715 |
| 94 | OBD168-q1069 | GTGGCAGGCTTCAGGGTGGTAGGACAGG | OBD168-q1071 |
| 95 | OBD168-q1513 | ATGAACACCCACAACGCCTGAACT | OBD168-q1515 |
| 96 | OBD168-q1933 | GTGGCTCACACCTGTAATCCC | OBD168-q1935 |
| 97 | OBD168-q1949 | ACACCTATAATCCCAGCACTTT | OBD168-q1951 |
| 98 | OBD168-q997 | TCCCCTGCCTCATCTTCCTCACCTGGC | OBD168-q999 |
| 99 | OBD168-q541 | GGACCTTTAGACATCGGTGGAGAACAGG | OBD168-q543 |
| 100 | OBD168-q2001 | GGCCAGCTGGAGTTCCAGGTGA | OBD168-q2003 |

TABLE 6.b8

| | | |
|---|---|---|
| 51 | AATCTCTTTGACTACACCATTCT | OBD168-p1737 |
| 52 | CAAACCCTTCATTTTTTATCCT | OBD168-p1989 |
| 53 | TCATTGAAATCTAAAATTCAG | OBD168-p2005 |
| 54 | TCCTTTAGAGCAACAAAAATGGA | OBD168-p1521 |
| 55 | CTTTCCTAGTCTATCATGTGTCTTTT | OBD168-p1369 |
| 56 | GCCCCTCACTTCCGCCACACTCCGTG | OBD168-p1137 |
| 57 | TGGGAGGCCAAGGTGGGAGGA | OBD168-p1885 |
| 58 | CAACAGCCTGACTCAATGACCAGTAAAG | OBD168-p953 |
| 59 | GCTGGGCGGATGGATGGCTTGAGTCTG | OBD168-p577 |
| 60 | CTCGGGTGCAGAGAGGAGGGTTTG | OBD168-p1937 |
| 61 | TAATTGGTACTTTCATGGGCCAAGAA | OBD168-p2013 |
| 62 | GCTTTCCACCCCTACAATCCCAAGTGTT | OBD168-p549 |
| 63 | GAGTCACAAAGCAGGAGGGAGAACAGGT | OBD168-p717 |
| 64 | GGGTGGTGAGGAAACACTAAGGAGAAGG | OBD168-p929 |
| 65 | CCCGGCCGACTCTTTTTTTTCTTAAT | OBD168-p2061 |
| 66 | ATTGAAATATCTGCCTTTATCTTA | OBD168-p1401 |
| 67 | CAGGCAACATCCCAGGGGCTGAAA | OBD168-p1373 |
| 68 | AAAACAACAAAACCCATAAAAT | OBD168-p1745 |
| 69 | GGGAGTGAGAAAGAGGAAGTATCAAATA | OBD168-p1253 |
| 70 | CTCCCGCCCCGCCCCGCCCCGGACA | OBD168-p1673 |
| 71 | CGCATAATCAAAGCCATCAGTGAC | OBD168-p1241 |
| 72 | CCCCTTCCAACCCAATCACCCCAC | OBD168-p1297 |
| 73 | AGAGCTAACATTTTATTCTGTGTT | OBD168-p2041 |
| 74 | GGGGGCATGGACCCGGCCTTGTTTG | OBD168-p1969 |
| 75 | AACAATAGAAATGACCCCTCAGGGTA | OBD168-p1601 |
| 76 | GCCTTTATGAGAAACCAGGCATTA | OBD168-p1493 |
| 77 | GGGGAAGGGACTCTCCTGGGGT | OBD168-p2097 |
| 78 | CTCCGTGTACTACTGGGCAAT | OBD168-p1789 |
| 79 | AGAACAAGAAGTCTGAGGAGAGTAG | OBD168-p1225 |
| 80 | CCCACCCACCTCCACCTGCCCACG | OBD168-p741 |
| 81 | TAAGGGCATTCTCCTAATAACTGTGATG | OBD168-p957 |
| 82 | CACAAGACTTCTGGCTTCTCAAGCC | OBD168-p1421 |
| 83 | GGAATCTGTGACATAGCATAT | OBD168-p1309 |
| 84 | TGAAGTTGTGCCAAAGCCACCTGTGCCA | OBD168-p505 |
| 85 | TAAAAGACCTGATGTATTTCCTACCAGC | OBD168-p593 |
| 86 | ACTTAAAGAACAGAATAGTTAAGTAA | OBD168-p1449 |
| 87 | GCTCTTGTGGAGAACTGGAGACCTTA | OBD168-p537 |
| 88 | CGGGTGCAGAGAGGAGGGTTTGGTG | OBD168-p1945 |
| 89 | GGACCTGAGTGCGAGGAGTTGTGTGGGC | OBD168-p609 |
| 90 | CTGTCTGGCTGGTGCTGGTGTCTCTG | OBD168-p1289 |
| 91 | GCGGGAGCCAGGGCAGAGAGACT | OBD168-p801 |
| 92 | CTAGAAACCATTAATCTACTATCTG | OBD168-p1753 |
| 93 | AGCCACGGCAGCCCCTGTAATCAACCCG | OBD168-p713 |
| 94 | GCTGCCTGCTGTTCCCGTGCCAG | OBD168-p1069 |
| 95 | ACCCCAAACACGGAGGCCCCAAGG | OBD168-p1513 |
| 96 | CCATATACTCATTTTTAAGCT | OBD168-p1933 |
| 97 | TGATAATATAATGTTATATTAA | OBD168-p1949 |
| 98 | CCTTGTTTCACCCCACACACTGACGGCG | OBD168-p997 |
| 99 | GTTGGCTGACTGTGAATGGTGGTAAAAT | OBD168-p541 |
| 100 | TACTAGTAAAAACATTACACTT | OBD168-p2001 |

TABLE 6.b9

| | | |
|---|---|---|
| 51 | TTGCTTGAGTTCAGGAGTTCGAACAAGATATTAAGTATTA | OBD168-p1739 |
| 52 | GTAGCTAGATACAGAGTGTCGATTGTCATGTTTTCACTGA | OBD168-p1991 |
| 53 | TCACCTGAGGTTTGGAGTTCGAATGTTTCTCTAATGCATG | OBD168-p2007 |
| 54 | AGGTGCTATGCTTATGACTCGAGACATGTGGAGTCACCCC | OBD168-p1523 |
| 55 | GTTAGCTCAGAGCCTCAGTCGAAGGAAGCTTAGGAATATA | OBD168-p1371 |
| 56 | AGGTCAGAAATTCGACCTCCTTGCTCAACTGTC | OBD168-p1139 |
| 57 | GTGTATGTCAACAATTTCTCGAACTCCCAGGTTCAAACGA | OBD168-p1887 |
| 58 | AGCTGGTCGGTATTCCCCTCGCTCTCGAGTAA | OBD168-p955 |
| 59 | AGGGAGTAGAGGAATCGGGAGCCAGTGTTCGAATAA | OBD168-p579 |
| 60 | TTTAGAGCTGTTTTCTCCTCGAAGGGCTGGTTGTACTGCG | OBD168-p1939 |
| 61 | GATCACTAGGTCAGGAGTTCGAAACCCACACTGCAAGTAT | OBD168-p2015 |
| 62 | AGGCAGGCGGATCACGAGGTCAATAGATCGATAGGA | OBD168-p551 |
| 63 | AGTTCACTGCAGCCTCGATACACATTTTCAAAGTGC | OBD168-p719 |
| 64 | TGAGCCCAGAAGTTCGAGTTTTCCCCTTGCTCC | OBD168-p931 |
| 65 | TCACTTGAGGTAAGGAGTTCGAAAATTAGCAGAAAAAAGC | OBD168-p2063 |
| 66 | TCACCTGAGTTCAGGAGTTCGAGATGACAGCTAAACATAA | OBD168-p1403 |
| 67 | GTTAGCTCAGAGCCTCAGTCGAGTCATATTTGAATGTCTA | OBD168-p1375 |
| 68 | ATCACTTGAGGTTAGGGTTCGACCAGCAGCAAAGACAGCA | OBD168-p1747 |
| 69 | TGCAGTGAGCCGAGATCATGCCATTATACTCGAAACAG | OBD168-p1255 |
| 70 | TCACTTGAGCCTAGGAAGTCGATGTTTCTTGAATTGTTTT | OBD168-p1675 |
| 71 | ATCACTTGAACCCGGGAGTCGAGATCAGCCTG | OBD168-p1243 |
| 72 | TGAGGTCAGAAATTCGAAACGAAGGGCTAAGCCCC | OBD168-p1299 |
| 73 | TCACTTGAGGCCAGGGATTCGATCCTCAAGCTAAGAATAA | OBD168-p2043 |
| 74 | TGCGTCCTGTCACTCTCCTCGAGGTTATATTTATTCTAAT | OBD168-p1971 |
| 75 | TTCTCCTGCCTCAGACTCTCGATACATAAGGACTTAGGAA | OBD168-p1603 |
| 76 | GATAAGCTTAAGAAGAGTTCGAGACACTTATTTTGGAGAT | OBD168-p1495 |
| 77 | TCACCTGAGGTCAGGAATTCGAAAAAAAAAAAAAAAAGAA | OBD168-p2099 |
| 78 | GTTAGCTCAGAGCCTCAGTCGAATACACACTCATTGAAGG | OBD168-p1791 |
| 79 | TGGGCAGATCACGAGGACAGGAGATCGAGCCT | OBD168-p1227 |
| 80 | TGTACATCGAGGTGAGAGTGAGCTGAGATGGCG | OBD168-p743 |
| 81 | AGGCAGATCATCTGAGGTTGGGAGTTCGAAGACAC | OBD168-p959 |
| 82 | CATAAGACTAAGGTTCTGTCGATACATATTTGTTTAATGA | OBD168-p1423 |
| 83 | ATGTTGGTCAGTCTGGTCTCGAAGTCACTCCTAACTCATA | OBD168-p1311 |
| 84 | AGCTTGTCTAGGTAAAGTTCCGGGTCGAATAGGCC | OBD168-p507 |
| 85 | AAGGCAGGTGGATCACGAGGTCAGCAGATCGATACATA | OBD168-p595 |
| 86 | TCACCTGAGGCCAGGAATTCGAAATACTATGCTAAGATTC | OBD168-p1451 |
| 87 | TAGGCCTCGAAAGGTCATTATAAGGATGGAGTGAG | OBD168-p539 |
| 88 | AAACATTAACTTGGTGTATCGAAGGGCTGGTTGTACTGCG | OBD168-p1947 |
| 89 | TGCAGGTGTGTGGTTAACACTATCCTTCGACCTGTAC | OBD168-p611 |
| 90 | AGTGGTGCCATCATAGCTCACTGCAGCCTCGAATAA | OBD168-p1291 |
| 91 | TGGCCAAGTGCACCACGTGAACAAAACAATCTCGATTGG | OBD168-p803 |
| 92 | ATCACTTGAGCTCAGGATTCGAAAACATTAACTGAAAGAA | OBD168-p1755 |
| 93 | ACGGAATGACATCGAGAAAGTGGAACTGGTAAGCATAGG | OBD168-p715 |
| 94 | AGGTGAGTTCGAGCTGCCGTGTGTCCCATAG | OBD168-p1071 |
| 95 | AGGTGCTATGCTTATGACTCGAGCTTGCACTCCGGTACCC | OBD168-p1515 |
| 96 | CAAGAGAATTGCTTGAACTCGATTCAGCATATGAAGGGGA | OBD168-p1935 |
| 97 | TTGCTTGAGCCTGAAAGGTCGAAAAATAAAAATGAAAATA | OBD168-p1951 |
| 98 | AGGCAGGAGGATCGCTTGAACCCAGGAGTTCGAAATA | OBD168-p999 |
| 99 | AGCCGCAAGAAGTACAGCAGCACCTCGATCTC | OBD168-p543 |
| 100 | AGCCTACCGGTGCTGCACTCGACAGAAAGCTTAAGGGCAA | OBD168-p2003 |

TABLE 6.b10

| | | |
|---|---|---|
| 51 | TAATACTTAATATCTTGTTCGAACTCCTGAACTCAAGCAA | ORF1_1_40249894_40253001_40397735_40401055_FR |
| 52 | TCAGTGAAAACATGACAATCGACACTCTGTATCTAGCTAC | ORF1_3_128892194_128893890_129064130_129068087_RF |
| 53 | CATGCATTAGAGAAACATTCGAACTCCAAACCTCAGGTGA | ORF1_4_113710897_113723443_113810658_113830867_RF |
| 54 | GGGGTGACTCCACATGTCTCGAGTCATAAGCATAGCACCT | ORF1_19_54627987_54632181_54828537_54832427_FR |
| 55 | TATATTCCTAAGCTTCCTTCGACTGAGGCTCTGAGCTAAC | ORF1_12_14189_15286_93751_97454_FF |
| 56 | AGACAGTTGAGCAAGGAGGTCGAATTTCTGACC | ORF1_13_27789091_27791217_27935736_27938158_RF |
| 57 | TCGTTTGAACCTGGGAGTTCGAGAAATTGTTGACATACAC | ORF1_19_6507876_6510412_6736179_6739196_RR |
| 58 | ACCATCTTCTGCCACTTTTACTCGAGAGCGAGGG | ORF1_5_71466175_71468826_71713282_71719473_RF |
| 59 | TTATTCGAACACTGGCTCCCGATTCCTCTACTCCC | ORF1_22_50201186_50205111_50373464_50374527_FF |
| 60 | CGCAGTACAACCAGCCCTTCGAGGAGAAAACAGCTCTAAA | ORF1_2_233813067_233815110_233853895_233857139_FR |
| 61 | ATACTTGCAGTGTGGGTTTCGAACTCCTGACCTAGTGATC | ORF1_4_168182554_168187989_168317980_168319647_FF |
| 62 | TCCTATCGATCTATTGACCTCGTGATCCGCCTGC | ORF1_2_101327421_101335518_101390331_101393544_FF |
| 63 | AGCACTTTGAAAATGTGTATCGAGGCTGCAGTGAAC | ORF1_1_221106643_221120931_221185158_221187933_RF |
| 64 | AAACTCGAACTTCTGGGCTCAAGCAATTCTCCTGCC | ORF1_15_31334951_31340691_31546136_31558743_FF |
| 65 | GCTTTTTTCTOGTAATTTTCGAACTCCTTACCTCAAGTGA | ORF1_7_43795674_43802582_44019092_44021471_RF |
| 66 | TTATGTTTAGCTGTCATCTCGAACTCCTGAACTCAGGTGA | ORF1_13_99494037_99497802_99735907_99738338_FF |
| 67 | TAGACATTCAAATATGACTCGACTGAGGCTCTGAGCTAAC | ORF1_12_14189_15286_200475_202854_FF |
| 68 | TGCTGTCTTTGCTGCTGGTCGAACCCTAACCTCAAGTGAT | ORF1_10_3653748_3655479_3866568_3872650_RR |
| 69 | TCCTGTTTCGAGTATAATGGCATGATCTCGGCTCACTG | ORF1_2_74446225_74448326_74574097_74579710_FR |
| 70 | AAAACAATTCAAGAAACATCGACTTCCTAGGCTCAAGTGA | ORF1_6_154995123_154998831_155125636_155128798_RF |
| 71 | TCAGGCTGATCTCGACTCCCGGGTTCAAGTGA | ORF1_19_9082207_9087550_9202787_9208911_RR |
| 72 | AGCCCTTCGTTTCGAATTTCTGACCTCAAGTGATCTGC | ORF1_13_27935736_27938158_27961367_27964197_FR |
| 73 | TTATTCTTAGCTTGAGGATCGAATCCCTGGCCTCAAGTGA | ORF1_6_158943910_158945125_159080063_159084244_RR |
| 74 | ATTAGAATAAATATAACCTCGAGGAGAGTGACAGGACGCA | ORF1_22_44263290_44267587_44326770_44330737_FF |
| 75 | TTCCTAAGTCCTTATGTATCGAGAGTCTGAGGCAGGAGAA | ORF1_3_32385454_32386698_32541292_32554994_RF |
| 76 | ATCTCCAAAATAAGTGTCTCGAACTCTTCTTAAGCTTATC | ORF1_19_20562776_20567807_20680600_20685168_RR |
| 77 | TTCTTTTTTTTTTTTTTTTCGAATTCCTGACCTCAGGTGA | ORF1_9_127592507_127594232_127769656_127771134_FR |
| 78 | CCTTCAATGAGTGTGTATTCGACTGAGGCTCTGAGCTAAC | ORF1_12_134194_137994_14189_15286_RF |
| 79 | TACAGGCTCGATCTCCTGTCCTCGTGATCTGC | ORF1_3_167588699_167598102_167735969_167742420_FF |
| 80 | TCTCAGCTCACTCTCACCTCGATGTACAAGACAG | ORF1_20_2111595_2115703_2369884_2374210_FR |
| 81 | TGTCTTCGAACTCCCAACCTCAGATGATCTGCCTG | ORF1_2_53649025_53656227_53766349_53775695_FR |
| 82 | TCATTAAACAAATATGTATCGACAGAACCTTAGTCTTATG | ORF1_16_70112537_70113568_70281463_70283063_FF |
| 83 | TATGAGTTAGGAGTGACTTCGAGACCAGACTGACCAACAT | ORF1_1_35943227_35949162_35991655_35992829_FF |
| 84 | AGGCCTATTCGACCCGGAACTTTACCTAGACAAG | ORF1_11_3589286_3591426_3777994_3782240_FF |
| 85 | TGTATCGATCTGCTGACCTCGTGATCCACCTGC | ORF1_13_40903938_40912685_40990073_40992867_FR |
| 86 | GAATCTTAGCATAGTATTTCGAATTCCTGGCCTCAGGTGA | ORF1_17_46177013_46180826_46195798_46197440_RF |
| 87 | ACTCCATCCTTATAATGACCTTTCGAGGCCTACCCC | ORF1_11_33667893_33674633_33693896_33698608_RR |
| 88 | CGCAGTACAACCAGCCCTTCGATACACCAAGTTAATGTTT | ORF1_2_233756599_233760219_233853895_233857139_FR |
| 89 | AGTACAGGTCGAAGGATAGTGTTAACCACACACCTGC | ORF1_21_44873351_44875256_44901291_44903482_FF |
| 90 | TTATTCGAGGCTGCAGTGAGCTATGATGGCACCAC | ORF1_1_39712316_39716819_39935069_39938243_RF |
| 91 | TCCCAATCGAGATTGTTTTGTTCACGTGGTGCACTTGG | ORF1_20_51449039_51452721_51482831_51487722_FF |
| 92 | TTCTTTCAGTTAATGTTTTCGAATCCTGAGCTCAAGTGAT | ORF1_11_10332545_10336410_10435291_10442726_FR |
| 93 | ACCCCTATGCTTACCAGTTCCACTTTCTCGATGTCATTC | ORF1_17_1305781_1308105_1405058_1407538_FR |
| 94 | TATGGGACACACGGCAGCTCGAACTCACCTG | ORF1_6_14276298_14278467_14395326_14400003_FR |
| 95 | GGGTACCGGAGTGCAAGCTCGAGTCATAAGCATAGCACCT | ORF1_19_54461246_54463256_54564139_54574977_RF |
| 96 | TCCCCTTCATATGCTGAATCGAGTTCAAGCAATTCTCTTG | ORF1_2_227722929_227725809_227932124_227939987_FR |
| 97 | TATTTTCATTTTTATTTTTCGACCTTTCAGGCTCAAGCAA | ORF1_20_20121190_20124157_20274659_20278486_RR |
| 98 | TATTTCGAACTCCTGGGTTCAAGCGATCCTCCTGC | ORF1_15_90843215_90845291_91002629_91006437_RR |
| 99 | TGAGATCGAGGTGCTGCTGTACTTCTTGCGGC | ORF1_22_24255117_24260015_24453446_24457028_FR |
| 100 | TTGCCCTTAAGCTTTCTGTCGAGTGCAGCACCGGTAGGCT | ORF1_4_102481630_102483332_102522760_102539034_FR |

TABLE 6.b11

| | | |
|---|---|---|
| 51 | OBD168-1737.1739 | OBD168-q1737.q1739, p1737/p1739 |
| 52 | OBD168-1989.1991 | OBD168-q1989.q1991, p1989/p1991 |
| 53 | OBD168-2005.2007 | OBD168-q2005.q2007, p2005/p2007 |
| 54 | OBD168-1521.1523 | OBD168-q1521.q1523, p1521/p1523 |
| 55 | OBD168-1369.1371 | OBD168-q1369.q1371, p1369/p1371 |
| 56 | OBD168-1137.1139 | OBD168-q1137.q1139, p1137/p1139 |
| 57 | OBD168-1885.1887 | OBD168-q1885.q1887, p1885/p1887 |
| 58 | OBD168-953.955 | OBD168-q953.q955, p953/p955 |
| 59 | OBD168-577.579 | OBD168-q577.q579, p577/p579 |
| 60 | OBD168-1937.1939 | OBD168-q1937.q1939, p1937/p1939 |
| 61 | OBD168-2013.2015 | OBD168-q2013.q2015, p2013/p2015 |
| 62 | OBD168-549.551 | OBD168-q549.q551, p549/p551 |
| 63 | OBD168-717.719 | OBD168-q717.q719, p717/p719 |
| 64 | OBD168-929.931 | OBD168-q929.q931, p929/p931 |
| 65 | OBD168-2061.2063 | OBD168-q2061.q2063, p2061/p2063 |
| 66 | OBD168-1401.1403 | OBD168-q1401.q1403, p1401/p1403 |
| 67 | OBD168-1373.1375 | OBD168-q1373.q1375, p1373/p1375 |
| 68 | OBD168-1745.1747 | OBD168-q1745.q1747, p1745/p1747 |
| 69 | OBD168-1253.1255 | OBD168-q1253.q1255, p1253/p1255 |
| 70 | OBD168-1673.1675 | OBD168-q1673.q1675, p1673/p1675 |
| 71 | OBD168-1241.1243 | OBD168-q1241.q1243, p1241/p1243 |
| 72 | OBD168-1297.1299 | OBD168-q1297.q1299, p1297/p1299 |
| 73 | OBD168-2041.2043 | OBD168-q2041.q2043, p2041/p2043 |
| 74 | OBD168-1969.1971 | OBD168-q1969.q1971, p1969/p1971 |
| 75 | OBD168-1601.1603 | OBD168-q1601.q1603, p1601/p1603 |
| 76 | OBD168-1493.1495 | OBD168-q1493.q1495, p1493/p1495 |
| 77 | OBD168-2097.2099 | OBD168-q2097.q2099, p2097/p2099 |
| 78 | OBD168-1789.1791 | OBD168-q1789.q1791, p1789/p1791 |
| 79 | OBD168-1225.1227 | OBD168-q1225.q1227, p1225/p1227 |
| 80 | OBD168-741.743 | OBD168-q741.q743, p741/p743 |
| 81 | OBD168-957.959 | OBD168-q957.q959, p957/p959 |
| 82 | OBD168-1421.1423 | OBD168-q1421.q1423, p1421/p1423 |
| 83 | OBD168-1309.1311 | OBD168-q1309.q1311, p1309/p1311 |
| 84 | OBD168-505.507 | OBD168-q505.q507, p505/p507 |
| 85 | OBD168-593.595 | OBD168-q593.q595, p593/p595 |
| 86 | OBD168-1449.1451 | OBD168-q1449.q1451, p1449/p1451 |
| 87 | OBD168-537.539 | OBD168-q537.q539, p537/p539 |
| 88 | OBD168-1945.1947 | OBD168-q1945.q1947, p1945/p1947 |
| 89 | OBD168-609.611 | OBD168-q609.q611, p609/p611 |
| 90 | OBD168-1289.1291 | OBD168-q1289.q1291, p1289/p1291 |
| 91 | OBD168-801.803 | OBD168-q801.q803, p801/p803 |
| 92 | OBD168-1753.1755 | OBD168-q1753.q1755, p1753/p1755 |
| 93 | OBD168-713.715 | OBD168-q713.q715, p713/p715 |
| 94 | OBD168-1069.1071 | OBD168-q1069.q1071, p1069/p1071 |
| 95 | OBD168-1513.1515 | OBD168-q1513.q1515, p1513/p1515 |
| 96 | OBD168-1933.1935 | OBD168-q1933.q1935, p1933/p1935 |
| 97 | OBD168-1949.1951 | OBD168-q1949.q1951, p1949/p1951 |
| 98 | OBD168-1000.998 | OBD168-q997.q999, p997/p999 |
| 99 | OBD168-541.543 | OBD168-q541.q543, p541/p543 |
| 100 | OBD168-2001.2003 | OBD168-q2001.q2003, p2001/p2003 |

TABLE 7.a1

| | Probe | E_SHAPLEY | L_SHAPLEY |
|---|---|---|---|
| 1 | ORF1_chr6_31267448_31269252_31531115_31534994_RF_1 | −0.009976177 | 0.009998253 |
| 2 | ORF1_chr20_50280026_50281924_50437155_50439548_FF_1 | −0.009046157 | 0.008905677 |
| 3 | ORF1_chr19_2777060_2780381_2901738_2905384_RF_1 | −0.008997425 | 0.008934787 |
| 4 | ORF1_chr6_134102628_134113539_134393136_134395072_RF_1 | −0.008843093 | 0.008908626 |
| 5 | ORF1_chr21_29092829_29094361_29164018_29174520_FF_1 | −0.008293347 | 0.008303626 |
| 6 | ORF1_chr22_23665784_23673822_23711932_23713061_FR_1 | −0.008166664 | 0.007845365 |
| 7 | ORF1_chrY_18283408_18293202_18412555_18418411_RR_1 | −0.008048342 | 0.007931165 |
| 8 | ORF1_chr2_218162078_218164150_218253352_218257050_FF_1 | −0.007932293 | 0.008034599 |
| 9 | ORF1_chr9_134315578_134321630_134391769_134398405_RF_1 | −0.007471656 | 0.007517793 |
| 10 | ORF1_chr20_31989698_31994926_32038678_32045816_RF_1 | −0.007474616 | 0.007497921 |
| 11 | ORF1_chr17_81104538_81109597_81345804_81351809_RR_1 | −0.007517468 | 0.007307065 |
| 12 | ORF1_chr22_41776313_41777375_42031915_42033846_RF_1 | −0.007370587 | 0.007409419 |
| 13 | ORF1_chr11_10637008_10641060_10826892_10830503_FR_1 | −0.007053792 | 0.007073053 |
| 14 | ORF1_chr19_4264058_4265818_4341821_4342968_FR_1 | −0.006933495 | 0.007060193 |
| 15 | ORF1_chr3_51243406_51244925_51388915_51391864_FF_1 | −0.007031162 | 0.006920945 |
| 16 | ORF1_chr11_10637008_10641060_10678778_10685244_FR_1 | −0.006833814 | 0.007048394 |
| 17 | ORF1_chr11_10562525_10569392_10617125_10621846_RR_1 | −0.006834039 | 0.007008485 |
| 18 | ORF1_chr2_74503923_74507137_74519716_74521847_RF_1 | −0.006920385 | 0.00691651 |
| 19 | ORF1_chr8_73772858_73775783_73991731_73996964_RF_1 | −0.006876044 | 0.006887353 |
| 20 | ORF1_chr21_29039171_29043861_29092829_29094361_FF_1 | −0.006945565 | 0.006784607 |
| 21 | ORF1_chr3_101887192_101889071_101943010_101950739_RF_1 | −0.006443814 | 0.006422371 |
| 22 | ORF1_chr6_29844477_29850988_29921307_29925901_FF_1 | −0.006005131 | 0.0059717 |
| 23 | ORF1_chr11_47655428_47658417_47713810_47715533_RF_1 | −0.005980292 | 0.005963403 |
| 24 | ORF1_chr11_10637008_10641060_10871146_10872290_FR_1 | −0.005962386 | 0.005929696 |
| 25 | ORF1_chr19_7270757_7273737_7367544_7370291_FR_1 | −0.005914296 | 0.005944901 |
| 26 | ORF1_chr19_4252394_4257822_4336234_4339292_FF_1 | −0.005861622 | 0.005979582 |
| 27 | ORF1_chr1_161657362_161661864_161734762_161740896_RF_1 | −0.005903164 | 0.005937478 |
| 28 | ORF1_chr11_65044650_65046952_65060361_65063993_FF_1 | −0.00587029 | 0.005934313 |
| 29 | ORF1_chr12_47686852_47690553_47817317_47827238_RF_1 | −0.005913599 | 0.005865812 |
| 30 | ORF1_chr5_690177_692906_744214_749963_FR_1 | −0.00548768 | 0.005562214 |
| 31 | ORF1_chr1_53182248_53184895_53364674_53371789_RF_1 | −0.005434164 | 0.005505088 |
| 32 | ORF1_chr15_62681808_62691633_62730771_62732716_RR_1 | −0.005498198 | 0.005387668 |
| 33 | ORF1_chr7_1769382_1772142_1917082_1921849_RF_1 | −0.004998178 | 0.005090942 |
| 34 | ORF1_chr16_57546345_57549019_57808053_57811713_RF_1 | −0.005033509 | 0.004962031 |
| 35 | ORF1_chr2_70887659_70889000_70979229_70981565_FF_1 | −0.005071083 | 0.004911433 |
| 36 | ORF1_chr9_123259504_123263022_123339568_123345302_FR_1 | −0.004932313 | 0.005024139 |
| 37 | ORF1_chrX_48750795_48754365_48796124_48801821_FF_1 | −0.005047329 | 0.004865913 |
| 38 | ORF1_chr1_158181667_158187628_158243613_158252050_FF_1 | −0.005001278 | 0.004859194 |
| 39 | ORF1_chr2_91859085_91863356_92122473_92125698_RF_1 | −0.004818038 | 0.005041727 |
| 40 | ORF1_chr10_62056179_62058893_62096285_62100217_RR_1 | −0.004920712 | 0.00492838 |
| 41 | ORF1_chr17_27740779_27745632_27849258_27851150_FR_1 | −0.005001185 | 0.004828046 |
| 42 | ORF1_chr7_41457346_41461355_41488360_41491403_FF_1 | −0.004417621 | 0.004430348 |
| 43 | ORF1_chr21_29039171_29043861_29092829_29094361_RF_1 | −0.004357641 | 0.0043654 |
| 44 | ORF1_chr20_50192390_50194162_50280026_50281924_RF_1 | −0.004385046 | 0.00427953 |
| 45 | ORF1_chr15_74686122_74687597_74772372_74779791_FR_1 | −0.004043082 | 0.003898205 |
| 46 | ORF1_chr18_63921019_63923125_64068334_64074049_RR_1 | −0.003835619 | 0.004088603 |
| 47 | ORF1_chr4_186172696_186175767_186190828_186192765_FF_1 | −0.003992969 | 0.003894199 |
| 48 | ORF1_chr17_67419703_67421985_67604580_67609081_RR_1 | −0.003838843 | 0.004030484 |
| 49 | ORF1_chr5_140242642_140251155_140386390_140387430_FF_1 | −0.003891098 | 0.003961009 |
| 50 | ORF1_chr22_42713609_42716051_42864112_42866876_FR_1 | −0.003934205 | 0.003914943 |

TABLE 7.a2

| | SHAPLEY_diff | RPs_class1_class2 | RPrank_class1_class2 | pfp_class1_class2 |
|---|---|---|---|---|
| 1 | 0.019974429 | 1095.305893 | 157 | 0.577069248 |
| 2 | 0.017951834 | 2212.25714 | 373 | 0.86431007 |
| 3 | 0.017932212 | 519.5921477 | 45 | 0.507750192 |
| 4 | 0.017751719 | 3237.196304 | 617 | 1.028118327 |
| 5 | 0.016596973 | 2338.87323 | 398 | 0.894701668 |
| 6 | 0.016012029 | 1286.837245 | 184 | 0.660136988 |
| 7 | 0.015979506 | 1545.88146 | 237 | 0.714351228 |
| 8 | 0.015966892 | 3812.00956 | 787 | 1.075173947 |
| 9 | 0.014989449 | 3730.532291 | 754 | 1.08039358 |
| 10 | 0.014972536 | 2973.092177 | 554 | 0.984923145 |
| 11 | 0.014824533 | 3166.187603 | 602 | 1.013212781 |
| 12 | 0.014780006 | 1915.600527 | 295 | 0.844353538 |
| 13 | 0.014126846 | 877.5566621 | 110 | 0.549057283 |
| 14 | 0.013993688 | 1462.635611 | 221 | 0.693140095 |
| 15 | 0.013952108 | 2161.353049 | 355 | 0.871099423 |
| 16 | 0.013882208 | 693.4429129 | 81 | 0.483049755 |
| 17 | 0.013842524 | 489.4037173 | 40 | 0.510408273 |
| 18 | 0.013836895 | 1139.344398 | 164 | 0.593584457 |
| 19 | 0.013763397 | 1188.857634 | 172 | 0.611542481 |
| 20 | 0.013730173 | 954.7111896 | 119 | 0.592329774 |
| 21 | 0.012866185 | 2281.449083 | 388 | 0.877911423 |
| 22 | 0.011976832 | 1854.11491 | 283 | 0.830066012 |
| 23 | 0.011943696 | 3360.825526 | 652 | 1.039518475 |
| 24 | 0.011892083 | 1629.314725 | 248 | 0.750594179 |
| 25 | 0.011859196 | 1122.566671 | 160 | 0.592200913 |
| 26 | 0.011841204 | 1414.449942 | 207 | 0.696494413 |
| 27 | 0.011840641 | 3341.384869 | 644 | 1.041707676 |
| 28 | 0.011804603 | 1538.948472 | 234 | 0.717660029 |
| 29 | 0.011779411 | 2485.195218 | 433 | 0.916381838 |
| 30 | 0.011049895 | 2340.89338 | 400 | 0.891601211 |
| 31 | 0.010939252 | 3294.7368 | 635 | 1.030563723 |
| 32 | 0.010885866 | 2954.724442 | 551 | 0.979467819 |
| 33 | 0.01008912 | 1465.773343 | 223 | 0.689591095 |
| 34 | 0.009995541 | 1026.72489 | 138 | 0.583437831 |
| 35 | 0.009982517 | 1455.232994 | 218 | 0.696259246 |
| 36 | 0.009956452 | 1242.495673 | 181 | 0.629677907 |
| 37 | 0.009913242 | 1020.280557 | 136 | 0.585246077 |
| 38 | 0.009860472 | 1130.926813 | 162 | 0.592850241 |
| 39 | 0.009859766 | 1980.300976 | 310 | 0.852847711 |
| 40 | 0.009849092 | 590.6246291 | 63 | 0.460927015 |
| 41 | 0.00982923 | 1226.644201 | 179 | 0.622029308 |
| 42 | 0.00884797 | 1511.98957 | 230 | 0.70719786 |
| 43 | 0.008723041 | 2252.095813 | 380 | 0.875891636 |
| 44 | 0.008664576 | 2050.835647 | 326 | 0.863503709 |
| 45 | 0.007941287 | 576.6524083 | 58 | 0.478789454 |
| 46 | 0.007924222 | 2662.422604 | 474 | 0.946326887 |
| 47 | 0.007887168 | 567.5154132 | 56 | 0.481315009 |
| 48 | 0.007869327 | 520.8353844 | 47 | 0.488329576 |
| 49 | 0.007852107 | 702.8036709 | 83 | 0.483257957 |
| 50 | 0.007849148 | 993.9971762 | 127 | 0.597522112 |

TABLE 7.a3

| | pval_class1_class2 | LogFC | FC |
|---|---|---|---|
| 1 | 0.000641938 | 0.54937122 | 1.46344773 |
| 2 | 0.00228425 | 0.514945922 | 1.42894058 |
| 3 | 0.000161893 | 0.734344848 | 1.66364181 |
| 4 | 0.004494626 | 0.527718822 | 1.44164787 |
| 5 | 0.002523054 | 0.492868642 | 1.40724024 |
| 6 | 0.000860631 | 0.517930598 | 1.43189986 |
| 7 | 0.001199569 | 0.48437531 | 1.39897997 |
| 8 | 0.005995408 | 0.483966176 | 1.39858329 |
| 9 | 0.005771898 | 0.494706192 | 1.40903377 |
| 10 | 0.003866138 | 0.597891561 | 1.51350303 |
| 11 | 0.004321778 | 0.545525036 | 1.45955142 |
| 12 | 0.001764866 | 0.53251218 | 1.44644572 |
| 13 | 0.000427933 | 0.611536519 | 1.52788559 |
| 14 | 0.001085372 | 0.4930395 | 1.40740691 |
| 15 | 0.002191096 | 0.535811103 | 1.449757 |
| 16 | 0.000277231 | 0.659020889 | 1.57901064 |
| 17 | 0.000144658 | 0.752486415 | 1.68469382 |
| 18 | 0.00068975 | 0.560959056 | 1.47524959 |
| 19 | 0.000745282 | 0.535735898 | 1.44968143 |
| 20 | 0.000499431 | 0.581573552 | 1.49648058 |
| 21 | 0.002413502 | 0.544133362 | 1.45814416 |
| 22 | 0.001664425 | 0.490257282 | 1.40469536 |
| 23 | 0.004802253 | 0.500493244 | 1.41469715 |
| 24 | 0.001318931 | 0.479953317 | 1.39469854 |
| 25 | 0.000671358 | 0.553684926 | 1.46783004 |
| 26 | 0.001021535 | 0.50212467 | 1.41629782 |
| 27 | 0.004753319 | 0.476374762 | 1.39124332 |
| 28 | 0.001189871 | 0.551237787 | 1.46534237 |
| 29 | 0.002811445 | 0.484667612 | 1.39926345 |
| 30 | 0.002526946 | 0.486152531 | 1.4007044 |
| 31 | 0.004636752 | 0.516035312 | 1.43001999 |
| 32 | 0.003823905 | 0.485297628 | 1.39987463 |
| 33 | 0.001089587 | 0.625064051 | 1.5422793 |
| 34 | 0.000570478 | 0.568005704 | 1.48247287 |
| 35 | 0.001075456 | 0.837265288 | 1.78666021 |
| 36 | 0.000807537 | 0.536000079 | 1.44994691 |
| 37 | 0.000563953 | 0.945939143 | 1.92644252 |
| 38 | 0.000680496 | 0.551565466 | 1.46567524 |
| 39 | 0.001873262 | 0.4917699 | 1.40616891 |
| 40 | 0.000205749 | 0.698897588 | 1.62326393 |
| 41 | 0.000788913 | 0.527326662 | 1.44125605 |
| 42 | 0.001152482 | 0.530273219 | 1.44420267 |
| 43 | 0.002358301 | 0.503295496 | 1.41744769 |
| 44 | 0.00199456 | 0.564237138 | 1.47860545 |
| 45 | 0.00019676 | 0.70669135 | 1.63205689 |
| 46 | 0.003178226 | 0.602479145 | 1.51832343 |
| 47 | 0.000190978 | 0.863602854 | 1.81957668 |
| 48 | 0.000162621 | 0.735559411 | 1.66504297 |
| 49 | 0.000284199 | 0.651399619 | 1.57069125 |
| 50 | 0.000537679 | 0.582993211 | 1.49795389 |

TABLE 7.b1

| | | | |
|---|---|---|---|
| 51 | ORF1_chr19_53278181_53279831_53467555_53470239_FR_1 | −0.003954476 | 0.003893624 |
| 52 | ORF1_chr2_43086424_43092123_43178166_43179822_FF_1 | −0.003899592 | 0.003872897 |
| 53 | ORF1_chr21_29066989_29073149_29092829_29094361_RF_1 | −0.003923032 | 0.003835069 |
| 54 | ORF1_chr8_143363265_143368758_143397073_143399812_RR_1 | −0.00348763 | 0.00354438 |
| 55 | ORF1_chr3_32625993_32627699_32683326_32685449_FF_1 | −0.003954396 | 0.002939267 |
| 56 | ORF1_chr4_2639345_2642002_2800083_2804902_RR_1 | −0.003409863 | 0.003468774 |
| 57 | ORF1_chr6_52573576_52576507_52715481_52719514_FR_1 | −0.002975625 | 0.003038734 |
| 58 | ORF1_chrX_39624253_39627672_39748652_39756826_RR_1 | −0.002950236 | 0.00301894 |
| 59 | ORF1_chr13_28585301_28589104_28625877_28627714_FR_1 | −0.002980592 | 0.00293983 |
| 60 | ORF1_chr6_15165975_15168155_15248586_15249747_RF_1 | −0.002979075 | 0.002914552 |
| 61 | ORF1_chr4_102249197_102251493_102344820_102348626_FR_1 | −0.002967096 | 0.00286827 |
| 62 | ORF1_chr2_10016688_10020163_10069588_10070662_RF_1 | −0.00282852 | 0.002999057 |
| 63 | ORF1_chr7_151781262_151783710_151930364_151932111_RR_1 | −0.002913771 | 0.00290113 |
| 64 | ORF1_chr1_26201425_26205480_26333363_26336159_FR_1 | −0.002798864 | 0.002984421 |
| 65 | ORF1_chr12_52037813_52040902_52237944_52243119_FR_1 | −0.002718844 | 0.002481841 |
| 66 | ORF1_chr19_41422837_41424323_41672917_41678014_RR_1 | −0.001958392 | 0.002928286 |
| 67 | ORF1_chr5_75205085_75210191_75236091_75239565_FR_1 | −0.002368141 | 0.002475733 |
| 68 | ORF1_chr10_104325690_104332059_104442837_104447074_RR_1 | −0.002480304 | 0.00207251 |
| 69 | ORF1_chr16_85533210_85536278_85655930_85660237_FR_1 | −0.001991499 | 0.002031361 |
| 70 | ORF1_chr17_43300561_43304860_43353578_43358798_RR_1 | −0.002056182 | 0.001965593 |
| 71 | ORF1_chr2_166059673_166070002_166163002_166177651_RF_1 | −0.001980001 | 0.001951355 |
| 72 | ORF1_chr20_50280026_50281924_50309417_50313312_FR_1 | −0.002011549 | 0.001887113 |
| 73 | ORF1_chr17_43300561_43304860_43444308_43448547_RR_1 | −0.001970743 | 0.001916479 |
| 74 | ORF1_chr11_10376885_10382511_10637008_10641060_RF_1 | −0.001949356 | 0.001899915 |
| 75 | ORF1_chr17_35363623_35366998_35578150_35580887_FF_1 | −0.001890538 | 0.001940668 |
| 76 | ORF1_chr8_41806799_41808430_41845432_41849175_RF_1 | −0.001900878 | 0.001918939 |
| 77 | ORF1_chrX_20268599_20270341_20456973_20459740_RR_1 | −0.002228408 | 0.001583268 |
| 78 | ORF1_chr15_76885704_76887654_76983002_76985852_RF_1 | −0.001424651 | 0.002248747 |
| 79 | ORF1_chr1_3073908_3076056_3169997_3177404_FF_1 | −0.001298227 | 0.002291357 |
| 80 | ORF1_chr2_112539682_112540702_112816387_112823836_RR_1 | −0.001408891 | 0.001734693 |
| 81 | ORF1_chr14_67469788_67473491_67514914_67519335_FR_1 | −0.001714218 | 0.001424876 |
| 82 | ORF1_chr17_20844189_20846843_20895269_20898565_RF_1 | −0.001475033 | 0.001468452 |
| 83 | ORF1_chr19_41422837_41424323_41597548_41599628_RR_1 | −0.001986899 | 0.000912203 |
| 84 | ORF1_chr11_35040602_35047084_35078579_35084103_FR_1 | −0.001430648 | 0.001433826 |
| 85 | ORF1_chr2_25155910_25160743_25292182_25293890_FR_1 | −0.001507045 | 0.001322379 |
| 86 | ORF1_chr9_70371691_70373189_70454780_70468182_RR_1 | −0.001420456 | 0.001407628 |
| 87 | ORF1_chr7_135917157_135920025_136070982_136076106_FR_1 | −0.001514277 | 0.000868748 |
| 88 | ORF1_chr18_45762052_45766373_45829537_45837293_FF_1 | −0.000956263 | 0.001417419 |
| 89 | ORF1_chr2_129732845_129738418_129813191_129815478_RF_1 | −0.000901332 | 0.001449501 |
| 90 | ORF1_chr3_122768838_122771609_122793452_122794847_FR_1 | −0.001347621 | 0.000921063 |
| 91 | ORF1_chr2_143119651_143128757_143274179_143284059_FR_1 | −0.001054557 | 0.001019704 |
| 92 | ORF1_chr19_19361989_19364321_19458777_19462243_RF_1 | −0.000988302 | 0.00107419 |
| 93 | ORF1_chr19_41422837_41424323_41692451_41693876_RR_1 | −0.000745499 | 0.001251804 |
| 94 | ORF1_chrX_101616906_101623004_101715574_101717864_FF_1 | −0.000996972 | 0.000984386 |
| 95 | ORF1_chr20_63754820_63756674_63899802_63901328_FF_1 | −0.000492369 | 0.000626577 |
| 96 | ORF1_chrX_39735882_39738523_39748652_39756826_FR_1 | −0.000676176 | 0.000418924 |
| 97 | ORF1_chr15_90688752_90691260_90881362_90884735_RF_1 | −0.000453442 | 0.000629736 |
| 98 | ORF1_chr16_8770523_8772703_8938508_8943672_RF_1 | −0.000319011 | 0.00074919 |
| 99 | ORF1_chr17_43300561_43304860_43406585_43411982_RF_1 | 0 | 0 |
| 100 | ORF1_chr7_1901148_1902503_2033188_2038434_RF_1 | 0 | 0 |

TABLE 7.b2

| | | | | |
|---|---|---|---|---|
| 51 | 0.0078481 | 2987.902851 | 560 | 0.982988691 |
| 52 | 0.007772489 | 493.6790445 | 43 | 0.48264036 |
| 53 | 0.007758101 | 2111.667632 | 341 | 0.869890982 |
| 54 | 0.00703201 | 1022.182606 | 137 | 0.582954788 |
| 55 | 0.006893663 | 2572.971635 | 459 | 0.919601928 |
| 56 | 0.006878637 | 1534.145351 | 233 | 0.716682261 |
| 57 | 0.00601436 | 1975.310453 | 307 | 0.857292939 |
| 58 | 0.005969176 | 3811.777698 | 786 | 1.076426753 |
| 59 | 0.005920422 | 886.7494008 | 112 | 0.549667601 |
| 60 | 0.005893626 | 1736.571207 | 266 | 0.784983374 |
| 61 | 0.005835366 | 3820.755115 | 790 | 1.075414102 |
| 62 | 0.005827578 | 1950.081799 | 300 | 0.857296534 |
| 63 | 0.005814901 | 2142.221032 | 350 | 0.869597722 |
| 64 | 0.005783285 | 367.3594761 | 26 | 0.452448159 |
| 65 | 0.005200686 | 1762.228575 | 269 | 0.79699158 |
| 66 | 0.004886678 | 1632.926185 | 249 | 0.75057046 |
| 67 | 0.004843873 | 2099.567055 | 337 | 0.871205394 |
| 68 | 0.004552814 | 2255.373656 | 383 | 0.871292473 |
| 69 | 0.00402286 | 1702.530556 | 259 | 0.777966877 |
| 70 | 0.004021776 | 1018.405989 | 133 | 0.59643941 |
| 71 | 0.003931356 | 1504.981553 | 228 | 0.707437155 |
| 72 | 0.003898662 | 1558.884246 | 241 | 0.713200465 |
| 73 | 0.003887222 | 2506.508269 | 436 | 0.924029095 |
| 74 | 0.003849271 | 1086.637744 | 154 | 0.579847865 |
| 75 | 0.003831206 | 2463.77808 | 427 | 0.915034411 |
| 76 | 0.003819816 | 1525.067865 | 232 | 0.712096468 |
| 77 | 0.003811676 | 3868.038973 | 811 | 1.070456089 |
| 78 | 0.003673398 | 2890.294194 | 533 | 0.97372438 |
| 79 | 0.003589584 | 3771.044431 | 770 | 1.078233722 |
| 80 | 0.003143584 | 2408.255424 | 414 | 0.906180999 |
| 81 | 0.003139094 | 2676.769491 | 478 | 0.947418348 |
| 82 | 0.002943484 | 3968.695995 | 832 | 1.091590126 |
| 83 | 0.002899102 | 1554.462101 | 239 | 0.715489386 |
| 84 | 0.002864474 | 1888.328908 | 290 | 0.837079106 |
| 85 | 0.002829424 | 3317.320304 | 640 | 1.034924223 |
| 86 | 0.002828085 | 3635.071398 | 727 | 1.070547766 |
| 87 | 0.002383025 | 2112.969893 | 342 | 0.868305311 |
| 88 | 0.002373682 | 2676.14117 | 477 | 0.949008348 |
| 89 | 0.002350832 | 3819.174236 | 789 | 1.075994131 |
| 90 | 0.002268684 | 2999.066738 | 563 | 0.984233204 |
| 91 | 0.002074262 | 2861.529024 | 528 | 0.965656771 |
| 92 | 0.002062493 | 3761.457058 | 768 | 1.07621306 |
| 93 | 0.001997303 | 3759.146475 | 766 | 1.077857615 |
| 94 | 0.001981358 | 3249.214339 | 622 | 1.026554835 |
| 95 | 0.001118946 | 4053.634633 | 867 | 1.087179498 |
| 96 | 0.001095099 | 3797.26634 | 778 | 1.080227905 |
| 97 | 0.001083178 | 2871.807678 | 529 | 0.969982617 |
| 98 | 0.0010682 | 4048.304309 | 865 | 1.08718107 |
| 99 | 0 | 4007.706758 | 846 | 1.092114447 |
| 100 | 0 | 2193.315447 | 362 | 0.876984215 |

TABLE 7.b3

| | | | |
|---|---|---|---|
| 51 | 0.003900334 | 0.489216266 | 1.40368213 |
| 52 | 0.000147047 | 0.74800396 | 1.6794676 |
| 53 | 0.002101767 | 0.521228516 | 1.43517684 |
| 54 | 0.000565875 | 1.172614248 | 2.25419801 |
| 55 | 0.002990734 | 0.49395581 | 1.40830109 |
| 56 | 0.001183172 | 0.537822012 | 1.45177916 |
| 57 | 0.001864803 | 0.560842558 | 1.47513047 |
| 58 | 0.005994767 | 0.526005779 | 1.43993709 |
| 59 | 0.000436198 | 0.618357963 | 1.53512695 |
| 60 | 0.001479474 | 0.552749686 | 1.46687881 |
| 61 | 0.006019606 | 0.477736691 | 1.3925573 |
| 62 | 0.00182229 | 0.530384363 | 1.44431394 |
| 63 | 0.002156511 | 0.562928095 | 1.47726443 |
| 64 | 8.33504E−05 | 0.839848798 | 1.78986255 |
| 65 | 0.001519047 | 0.788957054 | 1.72782494 |
| 66 | 0.001324208 | 0.488237425 | 1.40273008 |
| 67 | 0.002080251 | 0.480510844 | 1.39523762 |
| 68 | 0.002364438 | 0.555512609 | 1.46969074 |
| 69 | 0.001427664 | 0.573206678 | 1.48782689 |
| 70 | 0.000562061 | 0.610410685 | 1.52669374 |
| 71 | 0.001142847 | 0.518873573 | 1.43283608 |
| 72 | 0.00121785 | 0.482684355 | 1.39734122 |
| 73 | 0.002854548 | 0.719268743 | 1.64634734 |
| 74 | 0.000632703 | 0.553790771 | 1.46793773 |
| 75 | 0.002768411 | 0.520600136 | 1.43455187 |
| 76 | 0.001170556 | 0.501327357 | 1.41551531 |
| 77 | 0.006151131 | 0.491230225 | 1.40564299 |
| 78 | 0.003677295 | 0.689699991 | 1.61294807 |
| 79 | 0.005882594 | 0.49954823 | 1.41377078 |
| 80 | 0.002658157 | 0.791898345 | 1.73135113 |
| 81 | 0.003208743 | 0.5045375 | 1.41866849 |
| 82 | 0.006434995 | 0.508093184 | 1.42216927 |
| 83 | 0.00121162 | 0.482788771 | 1.39744235 |
| 84 | 0.001720005 | 0.587861355 | 1.50301703 |
| 85 | 0.004693035 | 0.563342168 | 1.47768849 |
| 86 | 0.005514495 | 0.480939987 | 1.39565271 |
| 87 | 0.002104088 | 0.528981192 | 1.44290988 |
| 88 | 0.003207404 | 0.585560668 | 1.50062206 |
| 89 | 0.006015229 | 0.520597798 | 1.43454955 |
| 90 | 0.003926193 | 0.652299468 | 1.57167124 |
| 91 | 0.003612618 | 0.642186187 | 1.56069236 |
| 92 | 0.005856319 | 0.505858365 | 1.41996795 |
| 93 | 0.005849994 | 0.497102018 | 1.41137564 |
| 94 | 0.004524158 | 0.57508128 | 1.48976139 |
| 95 | 0.006678603 | 0.479306005 | 1.3940729 |
| 96 | 0.005954705 | 0.480400377 | 1.39513079 |
| 97 | 0.003635674 | 0.520258621 | 1.43421233 |
| 98 | 0.006663206 | 0.474795884 | 1.38972158 |
| 99 | 0.006546419 | 0.486119667 | 1.4006725 |
| 100 | 0.002249394 | 0.53324977 | 1.44718541 |

TABLE 8.a1

| | Probe | E_SHAPLEY | L_SHAPLEY |
|---|---|---|---|
| 1 | ORF1_chr4_47908113_47912137_48013964_48015665_FF_1 | −0.010852058 | 0.010954909 |
| 2 | ORF1_chr2_230319552_230322976_230433766_230436650_RR_1 | −0.009918733 | 0.009925799 |
| 3 | ORF1_chr7_137931629_137933966_137981878_137983291_FR_1 | −0.009045837 | 0.008931235 |
| 4 | ORF1_chr12_5915876_5919160_6059110_6067436_FF_1 | −0.008949611 | 0.008980494 |
| 5 | ORF1_chr11_10776569_10779775_10900691_10903608_FF_1 | −0.008550855 | 0.008518259 |
| 6 | ORF1_chr10_45820548_45823847_45898343_45903899_RF_1 | −0.008438127 | 0.008469107 |
| 7 | ORF1_chr12_5915876_5919160_6152139_6154132_FF_1 | −0.008369285 | 0.008533464 |
| 8 | ORF1_chr3_124942088_124944537_125145442_125149201_RR_1 | −0.00807357 | 0.007991612 |
| 9 | ORF1_chr7_4620261_4621735_4724415_4726417_FR_1 | −0.008054585 | 0.007998688 |
| 10 | ORF1_chr1_7774817_7777030_7966809_7968736_FF_1 | −0.007973644 | 0.007968817 |
| 11 | ORF1_chr11_61042263_61044402_61243450_61246026_FF_1 | −0.007958072 | 0.007940873 |
| 12 | ORF1_chr5_55186470_55190634_55308382_55310405_FR_1 | −0.007008968 | 0.007035201 |
| 13 | ORF1_chr17_31222339_31224131_31324781_31330350_RR_1 | −0.007000437 | 0.007002552 |
| 14 | ORF1_chr4_38161792_38166364_38180538_38182770_FR_1 | −0.007017001 | 0.006967113 |
| 15 | ORF1_chr11_115616166_115618345_115711125_115715158_FF_1 | −0.00694982 | 0.007014028 |
| 16 | ORF1_chr8_7882531_7883805_7954782_7966362_FF_1 | −0.006887804 | 0.006999378 |
| 17 | ORF1_chr19_52960383_52965476_52976109_52979684_FR_1 | −0.006868407 | 0.006971455 |
| 18 | ORF1_chr12_7858281_7862390_7932397_7933511_RF_1 | −0.006878897 | 0.00695852 |
| 19 | ORF1_chr2_102051982_102057593_102268036_102274328_RR_1 | −0.006534163 | 0.006411507 |
| 20 | ORF1_chr2_27481760_27484387_27626184_27635183_RF_1 | −0.006497626 | 0.006363409 |
| 21 | ORF1_chr4_38133158_38135017_38161792_38166364_RF_1 | −0.00607496 | 0.005977457 |
| 22 | ORF1_chrX_2888191_2891631_3046105_3049187_RF_1 | −0.005996756 | 0.005958155 |
| 23 | ORF1_chr11_61224624_61227200_61378098_61379415_FF_1 | −0.005920792 | 0.00600365 |
| 24 | ORF1_chr10_13707193_13712319_13854651_13857653_FR_1 | −0.005932342 | 0.005964538 |
| 25 | ORF1_chr17_43832124_43833485_43905109_43906650_FF_1 | −0.005938095 | 0.005921624 |
| 26 | ORF1_chr11_12091217_12094358_12153671_12160688_FR_1 | −0.005949517 | 0.005909779 |
| 27 | ORF1_chr19_47777294_47778517_47957709_47959307_RF_1 | −0.005842488 | 0.005936168 |
| 28 | ORF1_chr13_30408291_30410407_30585632_30589891_RR_1 | −0.005735973 | 0.005672687 |
| 29 | ORF1_chr2_102100064_102105009_102374574_102377537_FR_1 | −0.005476339 | 0.005537616 |
| 30 | ORF1_chr8_97253466_97261198_97345458_97357412_RF_1 | −0.005440885 | 0.005548031 |
| 31 | ORF1_chr9_131999122_132006607_132139741_132142158_FR_1 | −0.005501932 | 0.005485202 |
| 32 | ORF1_chr22_16907330_16914217_17087415_17089015_RR_1 | −0.005393312 | 0.005512478 |
| 33 | ORF1_chr1_36159277_36165949_36303183_36304407_RF_1 | −0.005429147 | 0.005441681 |
| 34 | ORF1_chr11_61243450_61246026_61472770_61474691_FR_1 | −0.00527996 | 0.005431125 |
| 35 | ORF1_chr11_61152527_61154904_61243450_61246026_RF_1 | −0.005116399 | 0.004960923 |
| 36 | ORF1_chr13_94395452_94397500_94478315_94481582_FR_1 | −0.005019903 | 0.005001963 |
| 37 | ORF1_chr16_9911562_9916434_9954809_9955829_RF_1 | −0.004889205 | 0.005011129 |
| 38 | ORF1_chr2_174610120_174616390_174648154_174652062_RR_1 | −0.004979364 | 0.004908114 |
| 39 | ORF1_chr17_27477797_27480875_27629083_27630392_RR_1 | −0.004903841 | 0.004973785 |
| 40 | ORF1_chr10_91056658_91062643_91164372_91168358_RF_1 | −0.004802954 | 0.004928032 |
| 41 | ORF1_chr7_87727621_87737484_87868535_87872727_FF_1 | −0.004547573 | 0.004509927 |
| 42 | ORF1_chr15_30288521_30290733_30544055_30547496_FR_1 | −0.004473355 | 0.004505817 |
| 43 | ORF1_chr5_124631740_124634000_124857558_124858678_FR_1 | −0.004411595 | 0.00452833 |
| 44 | ORF1_chr22_17921708_17928023_17999187_18001605_FF_1 | −0.004480753 | 0.004446964 |
| 45 | ORF1_chr11_61243450_61246026_61388261_61391790_FR_1 | −0.004431923 | 0.004387812 |
| 46 | ORF1_chr4_74078231_74079386_74117911_74125141_FR_1 | −0.003947617 | 0.004031373 |
| 47 | ORF1_chr16_46899253_46904082_47060044_47061469_FF_1 | −0.003957876 | 0.004020226 |
| 48 | ORF1_chr11_17097711_17100441_17188135_17191681_RR_1 | −0.003990419 | 0.003979875 |
| 49 | ORF1_chrX_49053496_49056424_49286142_49288605_RF_1 | −0.004047095 | 0.003914997 |
| 50 | ORF1_chr12_51666821_51670814_51862305_51869704_FR_1 | −0.003954848 | 0.003996917 |

TABLE 8.a2

| | SHAPLEY_diff | RPs_class2_class1 | RPrank_class2_class1 | pfp_class2_class1 |
|---|---|---|---|---|
| 1 | 0.021806967 | 2796.807098 | 354 | 1.382985423 |
| 2 | 0.019844531 | 3095.482353 | 403 | 1.454244682 |
| 3 | 0.017977072 | 2644.935118 | 323 | 1.372546527 |
| 4 | 0.017930105 | 1190.76143 | 112 | 0.941892109 |
| 5 | 0.017069114 | 2691.77054 | 332 | 1.37767433 |
| 6 | 0.016907234 | 2598.185801 | 311 | 1.380989039 |
| 7 | 0.016902749 | 973.1243446 | 95 | 0.768375125 |
| 8 | 0.016065182 | 3118.518158 | 406 | 1.462565011 |
| 9 | 0.016053273 | 3166.931042 | 416 | 1.466844799 |
| 10 | 0.015942461 | 2534.406731 | 294 | 1.397614445 |
| 11 | 0.015898945 | 2203.942492 | 245 | 1.307037927 |
| 12 | 0.014044168 | 1277.819011 | 119 | 1.007768197 |
| 13 | 0.014002989 | 2501.124218 | 288 | 1.393528599 |
| 14 | 0.013984114 | 2829.764831 | 357 | 1.400184107 |
| 15 | 0.013963848 | 2842.705641 | 359 | 1.403705162 |
| 16 | 0.013887182 | 2337.981624 | 266 | 1.337778105 |
| 17 | 0.013839862 | 1105.727185 | 105 | 0.877882311 |
| 18 | 0.013837417 | 2568.815685 | 304 | 1.384488035 |
| 19 | 0.01294567 | 3724.636088 | 543 | 1.496046282 |
| 20 | 0.012861035 | 2452.218225 | 281 | 1.378853794 |
| 21 | 0.012052417 | 1684.416661 | 158 | 1.250937648 |
| 22 | 0.011954912 | 2093.173181 | 220 | 1.327259391 |
| 23 | 0.011924443 | 3162.182868 | 415 | 1.466482296 |
| 24 | 0.01189688 | 2875.544458 | 365 | 1.409057391 |
| 25 | 0.011859719 | 2395.722643 | 273 | 1.361478188 |
| 26 | 0.011859296 | 2559.990469 | 298 | 1.403736871 |
| 27 | 0.011778656 | 2875.54375 | 364 | 1.41292781 |
| 28 | 0.01140866 | 2592.51634 | 310 | 1.380068244 |
| 29 | 0.011013955 | 2033.960539 | 207 | 1.339926641 |
| 30 | 0.010988917 | 3139.677711 | 411 | 1.462162948 |
| 31 | 0.010987134 | 2077.891178 | 216 | 1.334213022 |
| 32 | 0.01090579 | 1885.344261 | 189 | 1.280763297 |
| 33 | 0.010870828 | 2319.480175 | 260 | 1.349373491 |
| 34 | 0.010711085 | 1776.408098 | 173 | 1.257251246 |
| 35 | 0.010077322 | 2615.722841 | 316 | 1.375504908 |
| 36 | 0.010021866 | 3181.350595 | 421 | 1.461116048 |
| 37 | 0.009900333 | 2003.48294 | 204 | 1.323326956 |
| 38 | 0.009887477 | 2020.977518 | 205 | 1.337558365 |
| 39 | 0.009877626 | 2916.46944 | 373 | 1.413834843 |
| 40 | 0.009730986 | 1780.548073 | 174 | 1.255271302 |
| 41 | 0.009057501 | 2722.114145 | 339 | 1.376378475 |
| 42 | 0.008979172 | 2927.700157 | 375 | 1.415911341 |
| 43 | 0.008939924 | 2362.930803 | 269 | 1.348160178 |
| 44 | 0.008927717 | 3062.496857 | 398 | 1.444849909 |
| 45 | 0.008819734 | 683.7987722 | 65 | 0.586539786 |
| 46 | 0.00797899 | 2461.265909 | 284 | 1.373274177 |
| 47 | 0.007978102 | 3384.670678 | 459 | 1.495157038 |
| 48 | 0.007970294 | 2640.403311 | 322 | 1.372615939 |
| 49 | 0.007962092 | 2072.613711 | 214 | 1.34056185 |
| 50 | 0.007951765 | 3075.288832 | 401 | 1.444662839 |

TABLE 8.a3

| | pval_class2_class1 | LogFC | FC |
|---|---|---|---|
| 1 | 0.003468855 | −0.482121919 | −1.396796568 |
| 2 | 0.004152482 | −0.466844878 | −1.382083594 |
| 3 | 0.003141195 | −0.522871523 | −1.436812218 |
| 4 | 0.000747454 | −0.619840252 | −1.536705015 |
| 5 | 0.003240783 | −0.497705955 | −1.411966593 |
| 6 | 0.003043098 | −0.500765128 | −1.414963784 |
| 7 | 0.000517204 | −0.638093589 | −1.556271307 |
| 8 | 0.004207329 | −0.467041118 | −1.382271601 |
| 9 | 0.004323573 | −0.502142409 | −1.416315236 |
| 10 | 0.002911387 | −0.494101348 | −1.408443165 |
| 11 | 0.002268922 | −0.521931704 | −1.435876536 |
| 12 | 0.000849714 | −0.58873139 | −1.503923716 |
| 13 | 0.002843634 | −0.502460069 | −1.416627122 |
| 14 | 0.003541756 | −0.478629571 | −1.393419416 |
| 15 | 0.003570554 | −0.518782064 | −1.432745201 |
| 16 | 0.002521338 | −0.506772102 | −1.420867576 |
| 17 | 0.000653117 | −0.619600182 | −1.536449321 |
| 18 | 0.00298214 | −0.493596886 | −1.407950766 |
| 19 | 0.005755859 | −0.463278915 | −1.378671665 |
| 20 | 0.0027453 | −0.553510489 | −1.467652572 |
| 21 | 0.001400419 | −0.567918634 | −1.482383403 |
| 22 | 0.00206892 | −0.519656066 | −1.433613438 |
| 23 | 0.004312114 | −0.464461297 | −1.379802039 |
| 24 | 0.003644071 | −0.476478708 | −1.391343564 |
| 25 | 0.002633532 | −0.581254338 | −1.496149497 |
| 26 | 0.002963925 | −0.503800653 | −1.417944096 |
| 27 | 0.003644069 | −0.507956161 | −1.422034199 |
| 28 | 0.00303129 | −0.490480928 | −1.404913131 |
| 29 | 0.001965245 | −0.532607428 | −1.446541214 |
| 30 | 0.004257973 | −0.495952972 | −1.41025199 |
| 31 | 0.002041946 | −0.621694416 | −1.538681271 |
| 32 | 0.001715126 | −0.623590728 | −1.540705079 |
| 33 | 0.002485826 | −0.577493645 | −1.492254542 |
| 34 | 0.001541109 | −0.558860668 | −1.47310541 |
| 35 | 0.003079743 | −0.489308066 | −1.403771448 |
| 36 | 0.00435845 | −0.466018016 | −1.381291697 |
| 37 | 0.001912769 | −0.637003815 | −1.555096184 |
| 38 | 0.001942817 | −0.585003283 | −1.500042403 |
| 39 | 0.003736567 | −0.476938298 | −1.391786866 |
| 40 | 0.001547576 | −0.537725962 | −1.451682509 |
| 41 | 0.003306 | −0.525337688 | −1.439270427 |
| 42 | 0.00376212 | −0.489523503 | −1.403981089 |
| 43 | 0.002569562 | −0.568458803 | −1.482938536 |
| 44 | 0.00407447 | −0.511994268 | −1.426020051 |
| 45 | 0.000270132 | −0.910817454 | −1.880110497 |
| 46 | 0.002763382 | −0.579204267 | −1.494024976 |
| 47 | 0.004862558 | −0.484711613 | −1.399306124 |
| 48 | 0.003131628 | −0.529384666 | −1.443313466 |
| 49 | 0.002032665 | −0.558880721 | −1.473125886 |
| 50 | 0.00410465 | −0.523083909 | −1.437023753 |

TABLE 8.b1

| | | | |
|---|---|---|---|
| 51 | ORF1_chr7_105801948_105809991_105902395_105905257_RF_1 | −0.003928487 | 0.004016509 |
| 52 | ORF1_chr11_95040575_95045594_95230445_95231653_FF_1 | −0.003774582 | 0.004068817 |
| 53 | ORF1_chr3_18693806_18695229_18731498_18743005_FR_1 | −0.003863946 | 0.003960599 |
| 54 | ORF1_chr10_44948033_44949598_44963508_44966433_RR_1 | −0.003433927 | 0.003436903 |
| 55 | ORF1_chr20_62658422_62663845_62934812_62935956_RF_1 | −0.003375218 | 0.003474561 |
| 56 | ORF1_chr12_131862265_131863764_132122269_132123632_RF_1 | −0.002996489 | 0.002955092 |
| 57 | ORF1_chr14_81530269_81532452_81602546_81604597_FF_1 | −0.002954625 | 0.002994667 |
| 58 | ORF1_chr11_61224624_61227200_61388261_61391790_FR_1 | −0.002931396 | 0.002976283 |
| 59 | ORF1_chr2_74503923_74507137_74519716_74521847_FF_1 | −0.002721172 | 0.002494579 |
| 60 | ORF1_chr14_74588744_74593680_74780483_74782393_FF_1 | −0.002345795 | 0.002725221 |
| 61 | ORF1_chr6_6745894_6751892_6983650_6986668_FR_l | −0.002489329 | 0.002493657 |
| 62 | ORF1_chr17_27395089_27399400_27471513_27474666_RR_1 | −0.003013145 | 0.001942483 |
| 63 | ORF1_chr1_42817616_42818863_42949613_42952408_FF_1 | −0.002429653 | 0.002517371 |
| 64 | ORF1_chr6_113632952_113639716_113727754_113732296_RF_1 | −0.002419275 | 0.002404141 |
| 65 | ORF1_chr1_109681178_109683602_109788010_109790308_FF_1 | −0.002316192 | 0.002187152 |
| 66 | ORF1_chr17_48576277_48578280_48760984_48766661_FF_1 | −0.001974644 | 0.002472355 |
| 67 | ORF1_chr1_203288566_203291154_203473865_203477190_FR_1 | −0.00239949 | 0.001969166 |
| 68 | ORF1_chr1_151547581_151552501_151755108_151762446_RF_1 | −0.002009281 | 0.002002169 |
| 69 | ORF1_chr12_109959287_109961500_109999941_110004320_FF_1 | −0.002045869 | 0.001951532 |
| 70 | ORF1_chr2_159230477_159238639_159250572_159255062_FF_1 | −0.00201036 | 0.001971956 |
| 71 | ORF1_chr12_121447890_121451709_121468997_121471919_FF_1 | −0.001954788 | 0.002009469 |
| 72 | ORF1_chr15_79006295_79007772_79123113_79127341_RF_1 | −0.001994597 | 0.001909468 |
| 73 | ORF1_chr5_40609009_40613245_40692971_40695207_FR_1 | −0.001862688 | 0.001974054 |
| 74 | ORF1_chr2_118112112_118113416_118185366_118187110_FR_1 | −0.001898732 | 0.001926152 |
| 75 | ORF1_chr7_22482163_22488443_22587350_22592472_RR_1 | −0.002213316 | 0.001441208 |
| 76 | ORF1_chr13_45338339_45340076_45393094_45398989_FR_1 | −0.001757898 | 0.001500113 |
| 77 | ORF1_chr17_82434207_82440644_82511425_82512972_FR_1 | −0.001779463 | 0.001444869 |
| 78 | ORF1_chr6_26459325_26460705_26493174_26499033_RR_1 | −0.001473288 | 0.001723885 |
| 79 | ORF1_chr16_81474879_81476453_81607034_81611259_RR_1 | −0.001444902 | 0.001747472 |
| 80 | ORF1_chr1_229299065_229300173_229506871_229512794_FR_1 | −0.001258617 | 0.001690872 |
| 81 | ORF1_chrX_77646855_77652042_77915112_77916580_RF_1 | −0.001439152 | 0.001489301 |
| 82 | ORF1_chr18_10287457_10291556_10505933_10507774_FF_1 | −0.001351442 | 0.001505109 |
| 83 | ORF1_chr16_18252588_18255195_18400348_18402976_RR_1 | −0.001457488 | 0.001379485 |
| 84 | ORF1_chr17_64322183_64329531_64384310_64385587_RF_1 | −0.001468917 | 0.000970412 |
| 85 | ORF1_chr1_109681178_109683602_109741801_109745521_FR_1 | −0.000854978 | 0.001473621 |
| 86 | ORF1_chr7_2130888_2136507_2376124_2377913_FF_1 | −0.000994991 | 0.001015707 |
| 87 | ORF1_chr6_86970655_86977787_87154540_87156275_RR_1 | −0.000999112 | 0.000981636 |
| 88 | ORF1_chr18_75155500_75159840_75214354_75217340_RF_1 | −0.000995619 | 0.000984043 |
| 89 | ORF1_chr5_179455074_179456297_179632102_179634386_RF_1 | −0.000963515 | 0.001005826 |
| 90 | ORF1_chr19_9777468_9779791_9828343_9833999_FF_1 | −0.000956186 | 0.000971537 |
| 91 | ORF1_chr7_100090606_100091968_100119089_100121546_RR_1 | −0.000846998 | 0.000978609 |
| 92 | ORF1_chr1_199677018_199683841_199811400_199820398_RR_1 | −0.000524121 | 0.000747654 |
| 93 | ORF1_chr14_81120330_81122262_81268937_81270783_RF_1 | −0.000751335 | 0.000481971 |
| 94 | ORF1_chr17_59893718_59895987_59916319_59918152_FR_1 | −0.000440873 | 0.000782713 |
| 95 | ORF1_chr5_94419239_94427299_94505842_94514405_FR_1 | −0.000742688 | 0.000456672 |
| 96 | ORF1_chr5_148804534_148807399_148901266_148906110_RF_1 | −0.000761756 | 0.000426533 |
| 97 | ORF1_chr1_185482953_185486658_185569901_185571399_FF_1 | −0.00070862 | 0.000438242 |
| 98 | ORF1_chr12_7868599_7870934_7932397_7933511_FF_1 | −0.000723781 | 0.000392015 |
| 99 | ORF1_chr2_111366352_111370512_111529947_111533147_FF_1 | −0.000252546 | 0.000793345 |
| 100 | ORF1_chr17_75411072_75413612_75649583_75651419_RF_1 | 0 | 0 |

TABLE 8.b2

| | | | | |
|---|---|---|---|---|
| 51 | 0.007944996 | 3184.944399 | 423 | 1.457114043 |
| 52 | 0.007843399 | 1598.706819 | 145 | 1.240602908 |
| 53 | 0.007824546 | 1311.207619 | 121 | 1.03860777 |
| 54 | 0.00687083 | 2075.653386 | 215 | 1.337834051 |
| 55 | 0.006849779 | 3172.19168 | 419 | 1.460623768 |
| 56 | 0.005951582 | 1895.333542 | 192 | 1.272773307 |
| 57 | 0.005949292 | 2604.836014 | 313 | 1.378421017 |
| 58 | 0.005907678 | 1898.912573 | 193 | 1.270475422 |
| 59 | 0.005215751 | 3577.121944 | 514 | 1.471925259 |
| 60 | 0.005071017 | 2923.537449 | 374 | 1.416119903 |
| 61 | 0.004982986 | 2654.348688 | 324 | 1.37698371 |
| 62 | 0.004955628 | 1345.935183 | 123 | 1.071320657 |
| 63 | 0.004947023 | 1670.587608 | 156 | 1.248294422 |
| 64 | 0.004823416 | 2749.196717 | 345 | 1.37644207 |
| 65 | 0.004503344 | 2769.148458 | 352 | 1.36651033 |
| 66 | 0.004447 | 3523.786793 | 500 | 1.473610575 |
| 67 | 0.004368655 | 3888.735935 | 577 | 1.518746877 |
| 68 | 0.00401145 | 2616.065515 | 317 | 1.371485397 |
| 69 | 0.0039974 | 2522.263056 | 292 | 1.39520081 |
| 70 | 0.003982317 | 3520.298247 | 498 | 1.476947968 |
| 71 | 0.003964256 | 676.3740979 | 64 | 0.583773394 |
| 72 | 0.003904066 | 3719.882512 | 541 | 1.498207138 |
| 73 | 0.003836743 | 2735.33663 | 341 | 1.380138694 |
| 74 | 0.003824884 | 424.4713289 | 42 | 0.371293764 |
| 75 | 0.003654525 | 3548.464077 | 504 | 1.480005974 |
| 76 | 0.003258011 | 2241.882572 | 247 | 1.336623574 |
| 77 | 0.003224333 | 3825.12433 | 567 | 1.501385344 |
| 78 | 0.003197172 | 3319.91788 | 449 | 1.477210974 |
| 79 | 0.003192374 | 3398.382345 | 463 | 1.49285337 |
| 80 | 0.002949489 | 2125.739433 | 224 | 1.340092929 |
| 81 | 0.002928453 | 3219.053584 | 430 | 1.46064713 |
| 82 | 0.002856551 | 2969.774836 | 382 | 1.425572936 |
| 83 | 0.002836973 | 2399.456066 | 274 | 1.360282508 |
| 84 | 0.002439329 | 3472.308017 | 483 | 1.486407014 |
| 85 | 0.002328598 | 2722.247784 | 340 | 1.372450033 |
| 86 | 0.002010698 | 2576.79482 | 308 | 1.374072688 |
| 87 | 0.001980748 | 2708.303887 | 335 | 1.380279235 |
| 88 | 0.001979662 | 2622.075007 | 319 | 1.368461872 |
| 89 | 0.001969341 | 3614.075373 | 525 | 1.467409322 |
| 90 | 0.001927724 | 2978.506253 | 383 | 1.429267903 |
| 91 | 0.001825607 | 3137.941194 | 410 | 1.464295228 |
| 92 | 0.001271775 | 3048.106352 | 395 | 1.443731983 |
| 93 | 0.001233306 | 2139.947545 | 225 | 1.35013943 |
| 94 | 0.001223586 | 3830.891167 | 568 | 1.50271561 |
| 95 | 0.001199359 | 3764.075451 | 551 | 1.501894245 |
| 96 | 0.001188289 | 2610.322544 | 315 | 1.374806784 |
| 97 | 0.001146861 | 3194.213302 | 426 | 1.45430681 |
| 98 | 0.001115797 | 1979.138215 | 201 | 1.313952351 |
| 99 | 0.001045891 | 2454.310373 | 282 | 1.376054571 |
| 100 | 0 | 3237.927886 | 434 | 1.462217536 |

TABLE 8.b3

| | | | |
|---|---|---|---|
| 51 | 0.004367161 | −0.465030208 | −1.380346255 |
| 52 | 0.001274577 | −0.550027026 | −1.464113123 |
| 53 | 0.000890435 | −0.847682935 | −1.799608311 |
| 54 | 0.002038008 | −0.563172098 | −1.477514304 |
| 55 | 0.004336283 | −0.512834609 | −1.426850922 |
| 56 | 0.00173148 | −0.680153457 | −1.602310181 |
| 57 | 0.003056972 | −0.557987109 | −1.472213708 |
| 58 | 0.001737356 | −0.542374915 | −1.456367968 |
| 59 | 0.005360609 | −0.485905118 | −1.400464213 |
| 60 | 0.00375264 | −0.553770679 | −1.467917288 |
| 61 | 0.003161106 | −0.548534801 | −1.462599528 |
| 62 | 0.000933662 | −0.737833914 | −1.667670091 |
| 63 | 0.001379771 | −0.56444409 | −1.47881757 |
| 64 | 0.003364669 | −0.543728094 | −1.457734612 |
| 65 | 0.003408167 | −0.552774164 | −1.4669037 |
| 66 | 0.005220571 | −0.472696176 | −1.387700446 |
| 67 | 0.006209069 | −0.462631594 | −1.378053209 |
| 68 | 0.003080461 | −0.559438786 | −1.473695833 |
| 69 | 0.002886588 | −0.587009685 | −1.502130011 |
| 70 | 0.005211465 | −0.485685281 | −1.400250828 |
| 71 | 0.000264722 | −0.672937759 | −1.594316169 |
| 72 | 0.005742942 | −0.474534292 | −1.389469621 |
| 73 | 0.00333459 | −0.483440859 | −1.39807413 |
| 74 | 0.000110492 | −0.832350253 | −1.780583699 |
| 75 | 0.005285174 | −0.477990632 | −1.392802437 |
| 76 | 0.002339221 | −0.643549918 | −1.56216833 |
| 77 | 0.006031711 | −0.464128291 | −1.379483586 |
| 78 | 0.004699527 | −0.48742988 | −1.401945125 |
| 79 | 0.004897376 | −0.49969168 | −1.413911362 |
| 80 | 0.002126906 | −0.61090833 | −1.527220454 |
| 81 | 0.004450195 | −0.515065261 | −1.429058783 |
| 82 | 0.003858496 | −0.553179285 | −1.467315677 |
| 83 | 0.002640857 | −0.605487251 | −1.52149253 |
| 84 | 0.005086864 | −0.488999758 | −1.403471491 |
| 85 | 0.003306288 | −0.49514089 | −1.409458393 |
| 86 | 0.002998649 | −0.587378889 | −1.502514474 |
| 87 | 0.00327625 | −0.568025828 | −1.48249355 |
| 88 | 0.003093062 | −0.561193944 | −1.475489797 |
| 89 | 0.005458532 | −0.466388598 | −1.381646553 |
| 90 | 0.003878624 | −0.473948075 | −1.388905146 |
| 91 | 0.004253807 | −0.528130529 | −1.442059337 |
| 92 | 0.004040629 | −0.529009247 | −1.442937935 |
| 93 | 0.002152417 | −0.665123047 | −1.585703511 |
| 94 | 0.006047702 | −0.463962152 | −1.379324736 |
| 95 | 0.00586349 | −0.464405972 | −1.379749126 |
| 96 | 0.003068439 | −0.586469733 | −1.50156792 |
| 97 | 0.00438966 | −0.46538094 | −1.380681871 |
| 98 | 0.001871289 | −0.527285374 | −1.441214801 |
| 99 | 0.002749477 | −0.603571085 | −1.519473048 |
| 100 | 0.004496421 | −0.464773079 | −1.380100261 |

TABLE 9.a1

| | Probe | pHC_SHAPLEY | SSc_SHAPLEY |
|---|---|---|---|
| 1 | ORF1_chr7_77473907_77477977_77652906_77658625_RR_1 | −0.015828606 | 0.015940502 |
| 2 | ORF1_chr12_117309542_117310883_117405729_117409691_FF_1 | −0.012158462 | 0.012202838 |
| 3 | ORF1_chr6_17759689_17762431_17990489_17995237_RR_1 | −0.011949649 | 0.011946795 |
| 4 | ORF1_chr11_11984245_11993733_12153671_12160688_RF_1 | −0.011885664 | 0.011918505 |
| 5 | ORF1_chr4_928981_931161_985198_989096_FF_1 | −0.01121672 | 0.011230981 |
| 6 | ORF1_chr6_10510556_10512653_10613542_10617081_RR_1 | −0.010471457 | 0.010499011 |
| 7 | ORF1_chr10_107634434_107636773_107740167_107742400_RR_1 | −0.009887937 | 0.010009661 |
| 8 | ORF1_chr12_117381529_117383570_117405729_117409691_FF_1 | −0.00991751 | 0.009875486 |
| 9 | ORF1_chr1_192936077_192944754_193111803_193113272_FF_1 | −0.00990311 | 0.009872441 |
| 10 | ORF1_chr7_8143002_8149481_8335072_8341646_RR_1 | −0.009424715 | 0.009402 |
| 11 | ORF1_chr6_109242018_109247965_109423758_109426226_RR_1 | −0.009397736 | 0.009406413 |
| 12 | ORF1_chr19_52598706_52601696_52670081_52671998_RR_1 | −0.008977625 | 0.00894306 |
| 13 | ORF1_chr20_45815058_45818479_46007260_46009918_FF_1 | −0.008796119 | 0.008978085 |
| 14 | ORF1_chr4_138242151_138246996_138326681_138334943_FF_1 | −0.008778706 | 0.008938522 |
| 15 | ORF1_chr11_12153671_12160688_12204447_12211177_FR_1 | −0.008841968 | 0.008870926 |
| 16 | ORF1_chr16_75377735_75379311_75564971_75566881_FF_1 | −0.008689436 | 0.008589716 |
| 17 | ORF1_chr11_11944920_11948043_12153671_12160688_RF_1 | −0.008026278 | 0.007972134 |
| 18 | ORF1_chr2_60549072_60551678_60708542_60711616_RF_1 | −0.007979329 | 0.007950747 |
| 19 | ORF1_chr12_57601753_57604119_57758134_57764996_FR_1 | −0.00783032 | 0.007933108 |
| 20 | ORF1_chr16_56302242_56305491_56354996_56357979_RF_1 | −0.007890141 | 0.007847278 |
| 21 | ORF1_chr5_126558745_126564482_126687692_126691508_FR_1 | −0.007703695 | 0.007744797 |
| 22 | ORF1_chr1_155206612_155209922_155288575_155290398_FR_1 | −0.006881006 | 0.007045115 |
| 23 | ORF1_chr7_37779378_37787306_37904027_37907035_FR_1 | −0.006984828 | 0.006941154 |
| 24 | ORF1_chr2_200711316_200714252_200958736_200960996_FR_1 | −0.006985272 | 0.006921379 |
| 25 | ORF1_chr6_17488412_17494115_17602256_17605423_FF_1 | −0.006944044 | 0.006954802 |
| 26 | ORF1_chr8_7810112_7811114_7862332_7864865_FR_1 | −0.006909953 | 0.006980468 |
| 27 | ORF1_chr11_36263501_36265092_36503363_36509375_RR_1 | −0.006916816 | 0.006930077 |
| 28 | ORF1_chr5_180083035_180085563_180303025_180304376_RF_1 | −0.006900089 | 0.006928636 |
| 29 | ORF1_chr20_45852988_45854096_46007260_46009918_FF_1 | −0.006886217 | 0.006939839 |
| 30 | ORF1_chr11_118438429_118442280_118496275_118499072_RR_1 | −0.006900764 | 0.006901984 |
| 31 | ORF1_chr11_57374233_57377459_57426633_57429019_FR_1 | −0.006854882 | 0.006939198 |
| 32 | ORF1_chr5_95749524_95751329_95836578_95843620_RF_1 | −0.006854744 | 0.006925527 |
| 33 | ORF1_chr2_233770451_233776693_233851260_233853895_RF_1 | −0.006350682 | 0.006440211 |
| 34 | ORF1_chr9_96135943_96140879_96269701_96274236_FF_1 | −0.006157529 | 0.006194649 |
| 35 | ORF1_chr14_90485762_90489489_90636665_90642057_FR_1 | −0.006049934 | 0.005992254 |
| 36 | ORF1_chr5_179958098_179961598_180083035_180085563_FR_1 | −0.005935888 | 0.006035068 |
| 37 | ORF1_chr5_74469407_74475515_74537627_74543794_RF_1 | −0.00589508 | 0.006021401 |
| 38 | ORF1_chr2_230865528_230871371_230949383_230957363_RR_1 | −0.005984927 | 0.005912069 |
| 39 | ORF1_chr2_60549072_60551678_60585452_60591312_RR_1 | −0.005875185 | 0.005979651 |
| 40 | ORF1_chr11_12153671_12160688_12359781_12366018_FR_1 | −0.005915796 | 0.005926733 |
| 41 | ORF1_chr1_192936077_192944754_193066855_193069148_FF_1 | −0.005908584 | 0.005912109 |
| 42 | ORF1_chr15_31214670_31223381_31397997_31400727_RF_1 | −0.00567441 | 0.005707802 |
| 43 | ORF1_chr7_102513081_102515918_102618627_102620296_RF_1 | −0.005113095 | 0.005142326 |
| 44 | ORF1_chr10_76940960_76942733_76997164_76999885_FF_1 | −0.005021227 | 0.004891893 |
| 45 | ORF1_chr1_20115093_20117021_20174195_20175240_RR_1 | −0.00494684 | 0.004951814 |
| 46 | ORF1_chr16_21532877_21535918_21626611_21627744_RR_1 | −0.004899085 | 0.004989605 |
| 47 | ORF1_chr10_103675840_103680824_103756732_103768333_FR_1 | −0.004951437 | 0.004880611 |
| 48 | ORF1_chr17_36104009_36106111_36150024_36152875_RR_1 | −0.004891181 | 0.004926395 |
| 49 | ORF1_chr12_124112544_124115693_124251083_124252846_FF_1 | −0.004938332 | 0.004867703 |
| 50 | ORF1_chr2_60549072_60551678_60704331_60707715_RR_1 | −0.004868576 | 0.004900767 |

TABLE 9.a2

| | SHAPLEY_diff | RPs_class1_class2 | RPrank_class1_class2 | pfp_class1_class2 |
|---|---|---|---|---|
| 1 | 0.031769108 | 4142.290351 | 671 | 0.088474811 |
| 2 | 0.0243613 | 799.5396006 | 102 | 0.00167783 |
| 3 | 0.023896444 | 6061.245091 | 1133 | 0.178943249 |
| 4 | 0.023804169 | 2024.157273 | 292 | 0.01756551 |
| 5 | 0.022447701 | 2294.052797 | 329 | 0.024204606 |
| 6 | 0.020970468 | 4986.41428 | 861 | 0.126289423 |
| 7 | 0.019897598 | 1498.254177 | 218 | 0.008022575 |
| 8 | 0.019792997 | 915.966393 | 126 | 0.00226586 |
| 9 | 0.019775551 | 3134.512644 | 475 | 0.049161058 |
| 10 | 0.018826715 | 2740.467886 | 399 | 0.03697672 |
| 11 | 0.018804148 | 2895.083912 | 434 | 0.041038607 |
| 12 | 0.017920684 | 2788.031445 | 410 | 0.038177056 |
| 13 | 0.017774204 | 2099.941321 | 305 | 0.019145107 |
| 14 | 0.017717228 | 1689.640917 | 240 | 0.011235739 |
| 15 | 0.017712894 | 1195.365584 | 175 | 0.004383924 |
| 16 | 0.017279152 | 1608.653115 | 232 | 0.009743215 |
| 17 | 0.015998412 | 1868.062483 | 269 | 0.014346301 |
| 18 | 0.015930076 | 1675.8149 | 239 | 0.010955144 |
| 19 | 0.015763427 | 1814.399786 | 257 | 0.013535854 |
| 20 | 0.01573742 | 2542.016781 | 360 | 0.031615997 |
| 21 | 0.015448492 | 1843.189907 | 264 | 0.013937125 |
| 22 | 0.013926121 | 247.895163 | 27 | 6.29435E−05 |
| 23 | 0.013925981 | 3030.39315 | 454 | 0.045852249 |
| 24 | 0.013906651 | 3000.314558 | 449 | 0.044813002 |
| 25 | 0.013898845 | 577.5597894 | 73 | 0.000676047 |
| 26 | 0.013890421 | 1193.63032 | 172 | 0.004436615 |
| 27 | 0.013846893 | 1213.237849 | 177 | 0.004577452 |
| 28 | 0.013828725 | 2971.913692 | 445 | 0.043772158 |
| 29 | 0.013826055 | 1561.169981 | 111 | 0.00893888 |
| 30 | 0.013802748 | 1915.265338 | 276 | 0.015278779 |
| 31 | 0.01379408 | 2583.552924 | 371 | 0.032448855 |
| 32 | 0.013780272 | 3218.60268 | 488 | 0.052345849 |
| 33 | 0.012790893 | 2715.586333 | 393 | 0.036381223 |
| 34 | 0.012352178 | 3195.533146 | 485 | 0.051401642 |
| 35 | 0.012042188 | 3140.736349 | 476 | 0.049389272 |
| 36 | 0.011970956 | 3412.75877 | 520 | 0.0598673 |
| 37 | 0.011916482 | 2886.148978 | 432 | 0.040794748 |
| 38 | 0.011896996 | 3687.643417 | 588 | 0.068646104 |
| 39 | 0.011854836 | 2741.589992 | 400 | 0.036936253 |
| 40 | 0.011842529 | 1843.29157 | 265 | 0.013887258 |
| 41 | 0.011820693 | 2773.668969 | 406 | 0.03787522 |
| 42 | 0.011382212 | 3926.448123 | 629 | 0.07907398 |
| 43 | 0.010255421 | 2330.127212 | 336 | 0.025028005 |
| 44 | 0.00991312 | 1559.386392 | 226 | 0.008941487 |
| 45 | 0.009898653 | 1621.68179 | 233 | 0.00998706 |
| 46 | 0.00988869 | 2371.450535 | 340 | 0.026297675 |
| 47 | 0.009832049 | 3074.121972 | 460 | 0.047516156 |
| 48 | 0.009817576 | 3316.432305 | 509 | 0.055533477 |
| 49 | 0.009806035 | 504.1606399 | 60 | 0.000485193 |
| 50 | 0.009769342 | 1396.243105 | 203 | 0.006670746 |

TABLE 9.a3

| | pval_class1_class2 | LogFC | FC |
|---|---|---|---|
| 1 | 0.000420637 | 0.44835068 | 1.364479462 |
| 2 | 1.21259E−06 | 0.622852779 | 1.539917198 |
| 3 | 0.001436516 | 0.622890966 | 1.539957959 |
| 4 | 3.6342E−05 | 0.507729916 | 1.421811212 |
| 5 | 5.64234E−05 | 1.005402106 | 2.007502947 |
| 6 | 0.000770434 | 0.427235363 | 1.344654345 |
| 7 | 1.23918E−05 | 0.52763826 | 1.441567369 |
| 8 | 2.02287E−06 | 0.595409615 | 1.510901505 |
| 9 | 0.000165455 | 0.426126481 | 1.343621216 |
| 10 | 0.000104536 | 0.483446371 | 1.398079472 |
| 11 | 0.000126197 | 0.448649187 | 1.364761815 |
| 12 | 0.000110905 | 0.441703694 | 1.358207302 |
| 13 | 4.13736E−05 | 0.479805041 | 1.3945552 |
| 14 | 1.91064E−05 | 0.515839018 | 1.429825432 |
| 15 | 5.43584E−06 | 0.561251237 | 1.475548393 |
| 16 | 1.60161E−05 | 0.534293195 | 1.448232463 |
| 17 | 2.73437E−05 | 0.515451081 | 1.429441007 |
| 18 | 1.85516E−05 | 0.524198 | 1.438133894 |
| 19 | 2.46481E−05 | 0.497471982 | 1.411737622 |
| 20 | 8.06445E−05 | 0.4514874 | 1.367449353 |
| 21 | 2.60701E−05 | 0.506296473 | 1.420399219 |
| 22 | 1.20415E−08 | 0.889807992 | 1.852929501 |
| 23 | 0.000147497 | 0.4436473 | 1.36003832 |
| 24 | 0.000142566 | 0.444190489 | 1.360550483 |
| 25 | 3.49675E−07 | 0.68443876 | 1.607076672 |
| 26 | 5.40686E−06 | 0.609011321 | 1.525213622 |
| 27 | 5.74067E−06 | 1.029600795 | 2.041459285 |
| 28 | 0.000138014 | 0.434077621 | 1.351046773 |
| 29 | 1.43772E−05 | 0.535011201 | 1.448953404 |
| 30 | 2.98788E−05 | 0.680580383 | 1.602784411 |
| 31 | 8.52979E−05 | 0.488098626 | 1.402595132 |
| 32 | 0.000180995 | 0.426429649 | 1.343903594 |
| 33 | 0.000101306 | 0.461288903 | 1.376771276 |
| 34 | 0.000176638 | 0.894733956 | 1.859266991 |
| 35 | 0.000166573 | 0.435112034 | 1.352015821 |
| 36 | 0.000220576 | 0.427789885 | 1.345171283 |
| 37 | 0.000124869 | 0.439979319 | 1.35658488 |
| 38 | 0.000285995 | 0.562968191 | 1.477305491 |
| 39 | 0.000104683 | 0.444287578 | 1.360642047 |
| 40 | 2.60752E−05 | 0.513498689 | 1.42750786 |
| 41 | 0.000108955 | 0.442026428 | 1.35851117 |
| 42 | 0.000352411 | 0.712879497 | 1.639072304 |
| 43 | 5.95842E−05 | 0.476336314 | 1.391206245 |
| 44 | 1.4318E−05 | 0.520855441 | 1.434805759 |
| 45 | 1.64877E−05 | 0.517907331 | 1.431876764 |
| 46 | 6.33522E−05 | 0.468615577 | 1.383780942 |
| 47 | 0.000154869 | 0.430435851 | 1.34764065 |
| 48 | 0.00020028 | 0.431443728 | 1.348582449 |
| 49 | 2.06268E−07 | 0.71006533 | 1.635878194 |
| 50 | 9.5948E−06 | 0.54514192 | 1.459163877 |

TABLE 9.b1

| | | | |
|---|---|---|---|
| 51 | ORF1_chr16_23959562_23960563_24103092_24104963_RR_1 | −0.004284513 | 0.004386812 |
| 52 | ORF1_chr11_68953615_68955511_69110685_69112785_FR_1 | −0.003933599 | 0.003924377 |
| 53 | ORF1_chr2_187296012_187300600_187333661_187349760_FF_1 | −0.003930151 | 0.003924756 |
| 54 | ORF1_chr17_79647743_79650208_79779269_79782236_RF_1 | −0.003873256 | 0.003917798 |
| 55 | ORF1_chr13_80048989_80053962_80108036_80110958_RR_1 | −0.00409337 | 0.003425493 |
| 56 | ORF1_chr11_12136334_12141132_12153671_12160688_FF_1 | −0.003573071 | 0.003044491 |
| 57 | ORF1_chr17_46172500_46175294_46370925_46372131_RF_1 | −0.002869624 | 0.003728354 |
| 58 | ORF1_chr7_127648061_127651700_127781428_127785589_RF_1 | −0.002831936 | 0.003651762 |
| 59 | ORF1_chr17_47945779_47949526_48160816_48163394_RR_1 | −0.002757089 | 0.00317307 |
| 60 | ORF1_chr1_67685932_67691400_67707953_67710845_RR_1 | −0.002884741 | 0.002956606 |
| 61 | ORF1_chr13_41335196_41337563_41355649_41358912_RR_1 | −0.002924545 | 0.002876178 |
| 62 | ORF1_chr1_2986093_2988416_3070581_3073621_RF_1 | −0.002938286 | 0.002820793 |
| 63 | ORF1_chr20_35273707_35277578_35323324_35324547_RR_1 | −0.002912582 | 0.002828771 |
| 64 | ORF1_chr8_72866427_72867681_72985368_72989505_FF_1 | −0.002480746 | 0.002887786 |
| 65 | ORF1_chr8_62981921_62989688_63002922_63008042_FF_1 | −0.002813177 | 0.002223156 |
| 66 | ORF1_chr2_112933066_112936593_113031116_113032593_RF_1 | −0.002179145 | 0.002847051 |
| 67 | ORF1_chr3_129012383_129018436_129244687_129248922_FR_1 | −0.002383172 | 0.002403262 |
| 68 | ORF1_chr12_10092308_10096371_10157054_10158072_RF_1 | −0.002404718 | 0.002074074 |
| 69 | ORF1_chr1_185478961_185481250_185562847_185565090_FF_1 | −0.00188049 | 0.00231512 |
| 70 | ORF1_chr11_12153671_12160688_12216760_12220209_FR_1 | −0.001981254 | 0.002049577 |
| 71 | ORF1_chr12_4049880_4053103_4104763_4106745_RF_1 | −0.001864302 | 0.001994257 |
| 72 | ORF1_chr4_123369547_123373273_123505935_123508246_RR_1 | −0.00189108 | 0.001848332 |
| 73 | ORF1_chr11_123648319_123653877_123684415_123688115_FF_1 | −0.001843695 | 0.001411908 |
| 74 | ORF1_chr17_46588843_46590052_46655874_46660167_FF_1 | −0.001432877 | 0.001729084 |
| 75 | ORF1_chr6_169127198_169132046_169290790_169293970_RF_1 | −0.001539251 | 0.001187329 |
| 76 | ORF1_chr1_160215823_160220940_160398180_160400303_FR_1 | −0.001542736 | 0.001158313 |
| 77 | ORF1_chr6_169127198_169132046_169286051_169290790_RF_1 | −0.001558311 | 0.001004034 |
| 78 | ORF1_chr14_106703042_106707057_106732214_106736829_RR_1 | −0.001236396 | 0.001311146 |
| 79 | ORF1_chr3_13931883_13934230_14046920_14054882_RR_1 | −0.001436492 | 0.000904925 |
| 80 | ORF1_chr12_12889292_12890821_12982042_12990253_FR_1 | −0.001257732 | 0.00084723 |
| 81 | ORF1_chrX_2276455_2282906_2372831_2374536_FF_1 | −0.000921368 | 0.000640653 |
| 82 | ORF1_chr12_427695_429020_611557_612617_FF_1 | −0.000569961 | 0.000952561 |
| 83 | ORF1_chr17_27528031_27530003_27672414_27678224_RR_1 | −0.000863285 | 0.000620921 |
| 84 | ORF1_chr16_11305532_11309621_11461548_11463016_RF_1 | −0.000536708 | 0.000727365 |
| 85 | ORF1_chr2_160492996_160495482_160608080_160611872_FF_1 | −0.000511574 | 0.000666378 |
| 86 | ORF1_chr10_5798770_5800647_6041802_6045506_RF_1 | −0.000538916 | 0.000607875 |
| 87 | ORF1_chr22_50531145_50533032_50776978_50783308_FR_1 | −0.000448306 | 0.000500907 |
| 88 | ORF1_chr8_140596381_140598037_140783960_140789447_RR_1 | −0.000406612 | 0.000371577 |
| 89 | ORF1_chr14_93886681_93889278_93972230_93975078_FR_1 | −0.000426731 | 0.00024599 |
| 90 | ORF1_chr1_93609850_93612271_93677337_93680831_FF_1 | −0.000384362 | 0.00014821 |
| 91 | ORF1_chr2_113266536_113267636_113504184_113510749_FF_1 | −0.000333362 | 0.000181115 |
| 92 | ORF1_chr6_36126333_36131280_36222214_36228907_RR_1 | −0.000292434 | 0.000215727 |
| 93 | ORF1_chr3_123064243_123067734_123161904_123162916_RF_1 | −0.000259212 | 7.66072E−05 |
| 94 | ORF1_chr7_143815670_143817160_143832880_143837398_FF_1 | 0 | 3.1633E−05 |
| 95 | ORF1_chr18_75239929_75247051_75392542_75394957_FF_1 | −1.94964E−05 | 0 |
| 96 | ORF1_chr9_37463569_37466721_37580276_37583007_RF_1 | −3.7519E−06 | 0 |
| 97 | ORF1_chr22_42535866_42536905_42603122_42604347_RR_1 | 0 | 2.54517E−06 |
| 98 | ORF1_chr16_30340361_30342037_30569930_30571793_FF_1 | 0 | −1.12902E−06 |
| 99 | ORF1_chr14_88868042_88879418_89003053_89008734_FF_1 | 0 | 9.75217E−07 |
| 100 | ORF1_chr1_21177377_21179201_21360997_21362872_RF_1 | 0 | 0 |

TABLE 9.b2

| | | | | |
|---|---|---|---|---|
| 51 | 0.008671324 | 4519.649801 | 754 | 0.10481268 |
| 52 | 0.007857976 | 2444.640804 | 346 | 0.028725693 |
| 53 | 0.007854907 | 3062.446524 | 458 | 0.047109718 |
| 54 | 0.007791053 | 2880.594219 | 429 | 0.040809999 |
| 55 | 0.007518863 | 2825.713915 | 419 | 0.039118584 |
| 56 | 0.006617562 | 1842.300047 | 263 | 0.013966084 |
| 57 | 0.006597977 | 3570.187804 | 557 | 0.065030297 |
| 58 | 0.006483699 | 5955.410877 | 1111 | 0.172610563 |
| 59 | 0.005930159 | 5412.489422 | 976 | 0.144987137 |
| 60 | 0.005841347 | 2011.449609 | 291 | 0.0172379 |
| 61 | 0.005800723 | 2982.959423 | 446 | 0.04423046 |
| 62 | 0.005759079 | 2470.335537 | 353 | 0.029197732 |
| 63 | 0.005741353 | 4585.205636 | 773 | 0.107154223 |
| 64 | 0.005368532 | 8881.585714 | 1969 | 0.335260652 |
| 65 | 0.005036332 | 6990.773109 | 1388 | 0.228289907 |
| 66 | 0.005026196 | 2546.429792 | 361 | 0.0317184 |
| 67 | 0.004786434 | 5541.80804 | 1005 | 0.151829671 |
| 68 | 0.004478793 | 7266.928609 | 1461 | 0.244545841 |
| 69 | 0.004195611 | 4271.30051 | 698 | 0.09408723 |
| 70 | 0.004030831 | 2226.338597 | 320 | 0.022407716 |
| 71 | 0.003858558 | 5249.941945 | 930 | 0.137999299 |
| 72 | 0.003739412 | 2894.809412 | 433 | 0.04112004 |
| 73 | 0.003255603 | 8318.413763 | 1775 | 0.304798022 |
| 74 | 0.003161961 | 2706.432558 | 391 | 0.036144627 |
| 75 | 0.002726581 | 1298.641194 | 194 | 0.005359081 |
| 76 | 0.002701048 | 7258.1481 | 1459 | 0.243967232 |
| 77 | 0.002562345 | 1109.170033 | 159 | 0.003661224 |
| 78 | 0.002547542 | 2223.082668 | 318 | 0.022433273 |
| 79 | 0.002341417 | 6875.273187 | 1351 | 0.222707957 |
| 80 | 0.002104962 | 4871.786943 | 831 | 0.121371898 |
| 81 | 0.001562021 | 3330.494838 | 511 | 0.056111863 |
| 82 | 0.001522522 | 6696.150841 | 1295 | 0.213997083 |
| 83 | 0.001484206 | 8356.225976 | 1789 | 0.30662177 |
| 84 | 0.001264073 | 10326.79147 | 2558 | 0.405690362 |
| 85 | 0.001177953 | 3774.294445 | 604 | 0.07220987 |
| 86 | 0.001146792 | 9736.38043 | 2312 | 0.376527839 |
| 87 | 0.000949213 | 8884.98958 | 1970 | 0.335479296 |
| 88 | 0.000778189 | 7143.652507 | 1434 | 0.236300225 |
| 89 | 0.000672721 | 11190.0887 | 2931 | 0.44902263 |
| 90 | 0.000532572 | 9652.970143 | 2270 | 0.37373372 |
| 91 | 0.000514477 | 7740.65394 | 1601 | 0.271112479 |
| 92 | 0.000508161 | 9560.939689 | 2228 | 0.369992569 |
| 93 | 0.00033582 | 11062.72759 | 2877 | 0.442278174 |
| 94 | 3.1633E−05 | 9517.746791 | 2208 | 0.368303263 |
| 95 | 1.94964E−05 | 5160.683472 | 911 | 0.133327303 |
| 96 | 3.7519E−06 | 8845.11861 | 1954 | 0.333652548 |
| 97 | 2.54517E−06 | 8336.699623 | 1781 | 0.305811136 |
| 98 | 1.12902E−06 | 5157.748755 | 907 | 0.133670574 |
| 99 | 9.75217E−07 | 4056.574955 | 647 | 0.085636774 |
| 100 | 0 | 8927.194783 | 1984 | 0.337922691 |

TABLE 9.b3

| | | | |
|---|---|---|---|
| 51 | 0.000559952 | 0.558649887 | 1.472890202 |
| 52 | 7.04226E−05 | 0.461871033 | 1.377326917 |
| 53 | 0.000152877 | 0.433791266 | 1.350778635 |
| 54 | 0.000124048 | 0.446075437 | 1.362329268 |
| 55 | 0.000116135 | 1.086848359 | 2.124095099 |
| 56 | 2.60253E−05 | 0.531412381 | 1.445343477 |
| 57 | 0.000256647 | 0.430040949 | 1.347271817 |
| 58 | 0.001358772 | 0.438276048 | 1.354984218 |
| 59 | 0.001002639 | 0.502224438 | 1.416395767 |
| 60 | 3.55421E−05 | 0.506724611 | 1.420820804 |
| 61 | 0.000139772 | 0.443199851 | 1.359616571 |
| 62 | 7.30279E−05 | 0.461479348 | 1.37695303 |
| 63 | 0.000586886 | 0.687683842 | 1.610695569 |
| 64 | 0.004677282 | 0.471794302 | 1.386833222 |
| 65 | 0.00224513 | 0.485572699 | 1.400141562 |
| 66 | 8.11304E−05 | 0.465214916 | 1.380522992 |
| 67 | 0.001081155 | 0.528459363 | 1.442388064 |
| 68 | 0.002531487 | 0.576268801 | 1.49098816 |
| 69 | 0.00046532 | 0.806605403 | 1.749091064 |
| 70 | 5.08057E−05 | 0.477341221 | 1.392175625 |
| 71 | 0.000909338 | 0.440182229 | 1.356775693 |
| 72 | 0.000126156 | 0.445571731 | 1.361853703 |
| 73 | 0.003833326 | 0.453615745 | 1.36946818 |
| 74 | 0.000100135 | 0.466067817 | 1.38133938 |
| 75 | 7.36643E−06 | 0.682695326 | 1.605135763 |
| 76 | 0.002522041 | 0.513378254 | 1.427388698 |
| 77 | 4.12467E−06 | 0.730988573 | 1.659776024 |
| 78 | 5.05458E−05 | 0.494873283 | 1.409196975 |
| 79 | 0.002131849 | 0.585302776 | 1.500353833 |
| 80 | 0.000714635 | 0.712422477 | 1.638553157 |
| 81 | 0.000203161 | 0.625281885 | 1.542512184 |
| 82 | 0.001963554 | 0.676867771 | 1.59866513 |
| 83 | 0.003886678 | 0.471014905 | 1.386084205 |
| 84 | 0.007352931 | 0.426007491 | 1.343510402 |
| 85 | 0.000309029 | 0.443981092 | 1.360353024 |
| 86 | 0.006168083 | 0.499525378 | 1.413748387 |
| 87 | 0.00468271 | 0.635619575 | 1.553604813 |
| 88 | 0.002400925 | 0.481023399 | 1.395733401 |
| 89 | 0.00932501 | 0.428795249 | 1.346109013 |
| 90 | 0.006011093 | 0.443625903 | 1.360018148 |
| 91 | 0.003075432 | 0.508132818 | 1.422208337 |
| 92 | 0.005840815 | 0.511154296 | 1.425190029 |
| 93 | 0.009015725 | 0.427129951 | 1.3445561 |
| 94 | 0.005761956 | 0.566380861 | 1.480804168 |
| 95 | 0.000860603 | 0.726262608 | 1.654347844 |
| 96 | 0.004619386 | 0.605470447 | 1.521474808 |
| 97 | 0.003859069 | 0.563812911 | 1.478170729 |
| 98 | 0.00085903 | 0.786926352 | 1.725394607 |
| 99 | 0.000392582 | 0.674090801 | 1.5955909 |
| 100 | 0.004750336 | 0.611416743 | 1.527758749 |

TABLE 10.a1

| | Probe | pHC_SHAPLEY | SSc_SHAPLEY |
|---|---|---|---|
| 1 | ORF1_chr2_8316756_8318269_8515734_8517409_RF_1 | −0.010970627 | 0.010951545 |
| 2 | ORF1_chr5_77940215_77942645_77971521_77974256_FF_1 | −0.010003787 | 0.009977067 |
| 3 | ORF1_chr4_113710897_113723443_113810658_113830867_RF_1 | −0.00997599 | 0.009952217 |
| 4 | ORF1_chr19_56561558_56564720_56759775_56765600_RF_1 | −0.009006777 | 0.009031113 |
| 5 | ORF1_chr2_92108138_92110573_92210006_92223089_FF_1 | −0.009012221 | 0.009019466 |
| 6 | ORF1_chr19_9082207_9087550_9202787_9208911_RR_1 | −0.008964267 | 0.009008719 |
| 7 | ORF1_chr6_14206770_14210218_14395326_14400003_RR_1 | −0.00874859 | 0.008702539 |
| 8 | ORF1_chr20_2111595_2115703_2369884_2374210_FR_1 | −0.00848945 | 0.008494726 |
| 9 | ORF1_chr10_3653748_3655479_3866568_3872650_RR_1 | −0.008511447 | 0.008398582 |
| 10 | ORF1_chr1_234729070_234732599_234814122_234817959_RF_1 | −0.008248522 | 0.00814939 |
| 11 | ORF1_chr13_40903938_40912685_40990073_40992867_FR_1 | −0.007988989 | 0.008046608 |
| 12 | ORF1_chr16_29013007_29015111_29280587_29285290_RF_1 | −0.007998535 | 0.008015258 |
| 13 | ORF1_chr3_32385454_32386698_32541292_32554994_RF_1 | −0.007954575 | 0.008047802 |
| 14 | ORF1_chr2_53649025_53656227_53766349_53775695_FR_1 | −0.00797809 | 0.008009025 |
| 15 | ORF1_chr1_165695181_165697826_165952803_165953958_FF_1 | −0.007968527 | 0.007940343 |
| 16 | ORF1_chrX_44917246_44925908_44940640_44941996_RR_1 | −0.007963647 | 0.007879094 |
| 17 | ORF1_chr1_221106643_221120931_221185158_221187933_RF_1 | −0.007992987 | 0.007810124 |
| 18 | ORF1_chr15_69554282_69555739_69599001_69607474_RF_1 | −0.007756475 | 0.007695703 |
| 19 | ORF1_chr15_90843215_90845291_91002629_91006437_RR_1 | −0.007731073 | 0.007687359 |
| 20 | ORF1_chr9_98247819_98251791_98381297_98383610_FR_1 | −0.007706678 | 0.007649644 |
| 21 | ORF1_chr19_6507876_6510412_6736179_6739196_RR_1 | −0.007483783 | 0.007411948 |
| 22 | ORF1_chr9_127592507_127594232_127769656_127771134_FR_1 | −0.00698097 | 0.007022252 |
| 23 | ORF1_chr4_168182554_168187989_168317980_168319647_FF_1 | −0.007014178 | 0.006934029 |
| 24 | ORF1_chr13_27789091_27791217_27935736_27938158_RF_1 | −0.00700985 | 0.006926581 |
| 25 | ORF1_chr8_57997832_57999789_58156560_58160085_FF_1 | −0.006989782 | 0.006917929 |
| 26 | ORF1_chr19_20562776_20567807_20680600_20685168_RR_1 | −0.006707615 | 0.006762372 |
| 27 | ORF1_chr1_39712316_39716819_39935069_39938243_RF_1 | −0.006729643 | 0.006693952 |
| 28 | ORF1_chr4_24971631_24973362_25125726_25129421_RR_1 | −0.006458105 | 0.006472968 |
| 29 | ORF1_chr1_234593513_234598020_234817959_234822547_FR_1 | −0.006243387 | 0.006170204 |
| 30 | ORF1_chr3_167588699_167598102_167735969_167742420_FF_1 | −0.006016394 | 0.006064867 |
| 31 | ORF1_chr20_20121190_20124157_20274659_20278486_RR_1 | −0.005995429 | 0.005970127 |
| 32 | ORF1_chr20_4141331_4145409_4240264_4242807_RF_1 | −0.006038388 | 0.00591904 |
| 33 | ORF1_chr2_28424743_28426988_28597316_28599022_FF_1 | −0.006004439 | 0.005917689 |
| 34 | ORF1_chr6_13871589_13875376_13978828_13980695_RR_1 | −0.006005242 | 0.005912011 |
| 35 | ORF1_chr1_117001584_117004481_117080679_117085351_RF_1 | −0.005995606 | 0.005921562 |
| 36 | ORF1_chr15_31334951_31340691_31546136_31558743_FF_1 | −0.006016007 | 0.005897539 |
| 37 | ORF1_chr19_54627987_54632181_54828537_54832427_FR_1 | −0.006012441 | 0.005879114 |
| 38 | ORF1_chr12_62435376_62437096_62567854_62572530_RR_1 | −0.005243132 | 0.005147521 |
| 39 | ORF1_chr6_14276298_14278467_14395326_14400003_FR_1 | −0.005212195 | 0.005137516 |
| 40 | ORF1_chr11_10332545_10336410_10435291_10442726_FR_1 | −0.005019572 | 0.00497803 |
| 41 | ORF1_chr1_35943227_35949162_35991655_35992829_FF_1 | −0.00498646 | 0.00497037 |
| 42 | ORF1_chr12_52899110_52901976_53003793_53005352_FF_1 | −0.004992319 | 0.004914104 |
| 43 | ORF1_chr6_154995123_154998831_155125636_155128798_RF_1 | −0.004995788 | 0.004902421 |
| 44 | ORF1_chr17_46177013_46180826_46195798_46197440_RF_1 | −0.004966559 | 0.004923055 |
| 45 | ORF1_chr14_74374067_74376797_74492554_74493993_RF_1 | −0.004990128 | 0.004857526 |
| 46 | ORF1_chr2_227722929_227725809_227932124_227939987_FR_1 | −0.004995629 | 0.004812622 |
| 47 | ORF1_chr15_69599001_69607474_69822688_69824653_FF_1 | −0.004973978 | 0.004830942 |
| 48 | ORF1_chr13_57155089_57159703_57172839_57181375_RR_1 | −0.004701299 | 0.004731776 |
| 49 | ORF1_chr6_14018522_14021640_14102931_14109639_RR_1 | −0.004732201 | 0.004690627 |
| 50 | ORF1_chr5_141346067_141348820_141380617_141383700_RR_1 | −0.004730581 | 0.004689889 |

TABLE 10.a2

| | SHAPLEY_diff | RPs_class2_class1 | RPrank_class2_class1 | pfp_class2_class1 |
|---|---|---|---|---|
| 1 | 0.021922172 | 2511.182773 | 159 | 0.068618829 |
| 2 | 0.019980854 | 2437.102121 | 153 | 0.064267578 |
| 3 | 0.019928208 | 2550.222683 | 166 | 0.069334424 |
| 4 | 0.01803789 | 699.403305 | 17 | 0.006055278 |
| 5 | 0.018031687 | 2380.232566 | 151 | 0.059981014 |
| 6 | 0.017972987 | 2935.649237 | 221 | 0.084519141 |
| 7 | 0.017451129 | 1813.287964 | 83 | 0.041820736 |
| 8 | 0.016984176 | 3175.82756 | 251 | 0.097260863 |
| 9 | 0.016910029 | 2880.834449 | 209 | 0.083791812 |
| 10 | 0.016397912 | 1642.123836 | 73 | 0.033344725 |
| 11 | 0.016035597 | 3199.669699 | 256 | 0.097809856 |
| 12 | 0.016013793 | 2210.422483 | 134 | 0.052181746 |
| 13 | 0.016002377 | 2979.923877 | 226 | 0.086983939 |
| 14 | 0.015987115 | 3112.832366 | 243 | 0.093857745 |
| 15 | 0.01590887 | 2535.270897 | 162 | 0.069613977 |
| 16 | 0.015842741 | 2034.612448 | 108 | 0.04836415 |
| 17 | 0.015803111 | 3457.695551 | 309 | 0.105280142 |
| 18 | 0.015452178 | 2523.803019 | 160 | 0.069385444 |
| 19 | 0.015418432 | 3285.787482 | 274 | 0.099977846 |
| 20 | 0.015356321 | 2639.438668 | 178 | 0.07282288 |
| 21 | 0.014895731 | 2684.363335 | 186 | 0.073868068 |
| 22 | 0.014003221 | 3053.776723 | 233 | 0.091712649 |
| 23 | 0.013948207 | 2678.622211 | 184 | 0.074121954 |
| 24 | 0.013936431 | 2611.734713 | 175 | 0.07142062 |
| 25 | 0.013907711 | 1277.907063 | 40 | 0.024505154 |
| 26 | 0.013469987 | 3074.794125 | 238 | 0.091906279 |
| 27 | 0.013423595 | 3274.528209 | 270 | 0.100288669 |
| 28 | 0.012931073 | 1204.291508 | 37 | 0.021309875 |
| 29 | 0.012413592 | 2105.950769 | 120 | 0.049152691 |
| 30 | 0.012081261 | 3070.236279 | 236 | 0.092218553 |
| 31 | 0.011965557 | 3330.318405 | 282 | 0.101659702 |
| 32 | 0.011957427 | 1907.921601 | 95 | 0.043787621 |
| 33 | 0.011922129 | 2263.050952 | 138 | 0.055023231 |
| 34 | 0.011917253 | 1968.695864 | 103 | 0.045136366 |
| 35 | 0.011917168 | 1818.554381 | 85 | 0.041261081 |
| 36 | 0.011913546 | 2745.03243 | 193 | 0.076882938 |
| 37 | 0.011891555 | 2600.984915 | 173 | 0.071223807 |
| 38 | 0.010390654 | 1932.678217 | 99 | 0.043985399 |
| 39 | 0.010349712 | 3398.290266 | 298 | 0.102982649 |
| 40 | 0.009997602 | 3268.369829 | 268 | 0.100396086 |
| 41 | 0.00995683 | 3168.607921 | 250 | 0.096899096 |
| 42 | 0.009906423 | 1844.510613 | 92 | 0.040095608 |
| 43 | 0.009898209 | 2915.081526 | 220 | 0.082886988 |
| 44 | 0.009889614 | 3200.455689 | 257 | 0.097510393 |
| 45 | 0.009847654 | 2363.728795 | 147 | 0.060136926 |
| 46 | 0.009808251 | 3350.237008 | 289 | 0.101213262 |
| 47 | 0.00980492 | 767.4793963 | 19 | 0.007714111 |
| 48 | 0.009433075 | 1450.88541 | 60 | 0.025946264 |
| 49 | 0.009422828 | 2550.977959 | 167 | 0.068989954 |
| 50 | 0.00942047 | 1843.602649 | 91 | 0.040465222 |

TABLE 10.a3

| | pval_class2_class1 | LogFC | FC |
|---|---|---|---|
| 1 | 7.73047E−05 | −0.48226793 | −1.396937941 |
| 2 | 6.96705E−05 | −0.489625593 | −1.404080442 |
| 3 | 8.15497E−05 | −0.479250266 | −1.394019039 |
| 4 | 7.29371E−07 | −0.682527783 | −1.604949366 |
| 5 | 6.41735E−05 | −0.492286246 | −1.406672271 |
| 6 | 0.000132347 | −0.461085729 | −1.376577399 |
| 7 | 2.45943E−05 | −0.553372605 | −1.46751231 |
| 8 | 0.000172973 | −0.454844133 | −1.370634715 |
| 9 | 0.000124083 | −0.466095029 | −1.381365435 |
| 10 | 1.72471E−05 | −0.585160477 | −1.500205854 |
| 11 | 0.000177414 | −0.450599782 | −1.366608289 |
| 12 | 4.95437E−05 | −0.499997327 | −1.414210942 |
| 13 | 0.000139288 | −0.45967456 | −1.375231562 |
| 14 | 0.0001616 | −0.454799064 | −1.370591898 |
| 15 | 7.99055E−05 | −0.481363125 | −1.396062108 |
| 16 | 3.70094E−05 | −0.509211229 | −1.423271831 |
| 17 | 0.0002305 | −0.472781967 | −1.387782969 |
| 18 | 7.86599E−05 | −0.483983089 | −1.398599688 |
| 19 | 0.000194097 | −0.445661056 | −1.361938026 |
| 20 | 9.18445E−05 | −0.481187162 | −1.395891843 |
| 21 | 9.73498E−05 | −0.477206877 | −1.392045991 |
| 22 | 0.000151409 | −0.457567534 | −1.37322453 |
| 23 | 9.6634E−05 | −0.473176258 | −1.388162304 |
| 24 | 8.85578E−05 | −0.477917751 | −1.392732078 |
| 25 | 6.94517E−06 | −0.575231366 | −1.489916383 |
| 26 | 0.000154984 | −0.458590251 | −1.374198345 |
| 27 | 0.000191858 | −0.447831463 | −1.363988483 |
| 28 | 5.5866E−06 | −0.624037056 | −1.541181801 |
| 29 | 4.17921E−05 | −0.517889633 | −1.431859199 |
| 30 | 0.000154204 | −0.455910163 | −1.371647873 |
| 31 | 0.000203125 | −0.44583572 | −1.362102923 |
| 32 | 2.94741E−05 | −0.520094882 | −1.434049558 |
| 33 | 5.3801E−05 | −0.497014203 | −1.411289737 |
| 34 | 3.29404E−05 | −0.545605491 | −1.459632816 |
| 35 | 2.48499E−05 | −0.523354058 | −1.437292865 |
| 36 | 0.000105136 | −0.470542609 | −1.385630516 |
| 37 | 8.73045E−05 | −0.479169234 | −1.393940744 |
| 38 | 3.08538E−05 | −0.526377578 | −1.440308223 |
| 39 | 0.000217443 | −0.446976729 | −1.363180618 |
| 40 | 0.000190641 | −0.447477712 | −1.363654072 |
| 41 | 0.000171643 | −0.452052508 | −1.367985092 |
| 42 | 2.61366E−05 | −0.543175273 | −1.457176135 |
| 43 | 0.000129204 | −0.462069059 | −1.377515984 |
| 44 | 0.000177562 | −0.450330176 | −1.366352925 |
| 45 | 6.2636E−05 | −0.495100573 | −1.409419005 |
| 46 | 0.000207253 | −0.446404322 | −1.362639868 |
| 47 | 1.0385E−06 | −0.66472302 | −1.585263892 |
| 48 | 1.10304E−05 | −0.621733538 | −1.538722995 |
| 49 | 8.16333E−05 | −0.496666608 | −1.41094975 |
| 50 | 2.60909E−05 | −0.559476508 | −1.473734365 |

TABLE 10.b1

| | | | |
|---|---|---|---|
| 51 | ORF1_chr21_44873351_44875256_44901291_44903482_FF_1 | −0.004503859 | 0.004600811 |
| 52 | ORF1_chr11_33667893_33674633_33693896_33698608_RR_1 | −0.004478153 | 0.004366546 |
| 53 | ORF1_chr1_89127656_89129856_89172214_89177508_RR_1 | −0.004254783 | 0.004311819 |
| 54 | ORF1_chr2_233813067_233815110_233853895_233857139_FR_1 | −0.004241178 | 0.004324562 |
| 55 | ORF1_chr2_101327421_101335518_101390331_101393544_FF_1 | −0.004033275 | 0.00395755 |
| 56 | ORF1_chr2_74446225_74448326_74574097_74579710_FR_1 | −0.003993608 | 0.003972901 |
| 57 | ORF1_chr13_99494037_99497802_99735907_99738338_FF_1 | −0.003979494 | 0.003967993 |
| 58 | ORF1_chr19_54789678_54792948_54890303_54892564_RF_1 | −0.003994289 | 0.003899119 |
| 59 | ORF1_chr11_3589286_3591426_3777994_3782240_FF_1 | −0.003982896 | 0.003890388 |
| 60 | ORF1_chr1_224094766_224099331_224328317_224334418_FF_1 | −0.003967915 | 0.003902095 |
| 61 | ORF1_chr5_54687073_54701690_54895820_54899919_RF_1 | −0.003934966 | 0.003930505 |
| 62 | ORF1_chr2_54954222_54956881_55045882_55048981_FF_1 | −0.003962377 | 0.003875283 |
| 63 | ORF1_chr16_70112537_70113568_70281463_70283063_FF_1 | −0.003722041 | 0.003780142 |
| 64 | ORF1_chr1_89127656_89129856_89152351_89155427_RR_1 | −0.003742763 | 0.003444532 |
| 65 | ORF1_chr22_29467180_29469328_29549133_29556322_FR_1 | −0.003511432 | 0.003502623 |
| 66 | ORF1_chr4_24971631_24973362_25161538_25167741_RR_1 | −0.003426172 | 0.003412223 |
| 67 | ORF1_chr6_33982531_33984798_34087216_34091346_RF_1 | −0.003018538 | 0.003374011 |
| 68 | ORF1_chr1_16764579_16768424_17044824_17050263_RR_1 | −0.002974774 | 0.003374112 |
| 69 | ORF1_chr19_54461246_54463256_54564139_54574977_RF_1 | −0.00298649 | 0.0030144 |
| 70 | ORF1_chr3_128892194_128893890_129064130_129068087_RF_1 | −0.003027572 | 0.002930318 |
| 71 | ORF1_chr8_8984061_8987615_9203064_9212772_RR_1 | −0.003009832 | 0.002941725 |
| 72 | ORF1_chr16_70112537_70113568_70359934_70360962_FR_1 | −0.002959526 | 0.002989236 |
| 73 | ORF1_chr13_112765663_112769601_112883502_112886179_FR_1 | −0.002760106 | 0.003186397 |
| 74 | ORF1_chr2_92108138_92110573_92242402_92244832_FF_1 | −0.00301196 | 0.002900616 |
| 75 | ORF1_chr22_24255117_24260015_24453446_24457028_FR_1 | −0.002807104 | 0.002724808 |
| 76 | ORF1_chr5_71466175_71468826_71713282_71719473_RF_1 | −0.002236045 | 0.00279856 |
| 77 | ORF1_chr22_44263290_44267587_44326770_44330737_FF_1 | −0.002217184 | 0.002806291 |
| 78 | ORF1_chr2_233756599_233760219_233853895_233857139_FR_1 | −0.002232479 | 0.002440345 |
| 79 | ORF1_chr5_77232748_77239462_77325709_77329000_RF_1 | −0.002455558 | 0.002195437 |
| 80 | ORF1_chr7_43795674_43802582_44019092_44021471_RF_1 | −0.001987004 | 0.002067425 |
| 81 | ORF1_chr13_112592963_112600705_112765663_112769601_RF_1 | −0.002016484 | 0.002005472 |
| 82 | ORF1_chr19_54627987_54632181_54748746_54753675_FF_1 | −0.001940709 | 0.00203635 |
| 83 | ORF1_chr4_102481630_102483332_102522760_102539034_FR_1 | −0.001978781 | 0.001923158 |
| 84 | ORF1_chr6_158943910_158945125_159080063_159084244_RR_1 | −0.001961303 | 0.001933679 |
| 85 | ORF1_chr22_50201186_50205111_50373464_50374527_FF_1 | −0.00196373 | 0.001893493 |
| 86 | ORF1_chr9_98247819_98251791_98368594_98373451_FF_1 | −0.001727011 | 0.00169324 |
| 87 | ORF1_chr1_33081752_33087352_33257774_33262528_RR_1 | −0.001501051 | 0.001889256 |
| 88 | ORF1_chr12_134194_137994_14189_15286_RF_1 | −0.00149159 | 0.001885713 |
| 89 | ORF1_chr5_77219829_77221017_77232748_77239462_FR_1 | −0.001687268 | 0.001671906 |
| 90 | ORF1_chr5_76987562_76988733_77232748_77239462_RR_1 | −0.001478176 | 0.001851719 |
| 91 | ORF1_chr1_40249894_40253001_40397735_40401055_FR_1 | −0.001902953 | 0.001370112 |
| 92 | ORF1_chr6_33937395_33940607_33982531_33984798_RF_1 | −0.001501752 | 0.001504049 |
| 93 | ORF1_chr20_51449039_51452721_51482831_51487722_FF_1 | −0.001492636 | 0.00144743 |
| 94 | ORF1_chr12_14189_15286_93751_97454_FF_1 | −0.00124942 | 0.001308707 |
| 95 | ORF1_chr13_27935736_27938158_27961367_27964197_FR_1 | −0.001006841 | 0.000936839 |
| 96 | ORF1_chr6_33846691_33848820_33982531_33984798_RF_1 | −0.000722866 | 0.000940477 |
| 97 | ORF1_chr6_34087216_34091346_34298424_34301201_FR_1 | −0.000722143 | 0.000925297 |
| 98 | ORF1_chr17_1305781_1308105_1405058_1407538_FR_1 | 0 | 5.39744E−05 |
| 99 | ORF1_chr12_14189_15286_200475_202854_FF_1 | −2.95539E−05 | 0 |
| 100 | ORF1_chr16_86891168_86894116_87066023_87067839_FF_1 | 0 | −5.49626E−07 |

TABLE 10.b2

| | | | | |
|---|---|---|---|---|
| 51 | 0.009104671 | 3277.035573 | 271 | 0.100177474 |
| 52 | 0.008844699 | 3274.351845 | 269 | 0.100643162 |
| 53 | 0.008566602 | 2141.773756 | 128 | 0.048898615 |
| 54 | 0.00856574 | 2795.051917 | 200 | 0.078941759 |
| 55 | 0.007990825 | 2686.831942 | 187 | 0.073706177 |
| 56 | 0.007966509 | 2938.850856 | 222 | 0.084452402 |
| 57 | 0.007947488 | 2781.085401 | 197 | 0.078776945 |
| 58 | 0.007893408 | 1442.042553 | 56 | 0.027189805 |
| 59 | 0.007873284 | 3336.811355 | 285 | 0.101252915 |
| 60 | 0.007870009 | 1350.536348 | 47 | 0.02552298 |
| 61 | 0.007865471 | 1300.310218 | 42 | 0.024870375 |
| 62 | 0.00783766 | 2359.189043 | 145 | 0.060559097 |
| 63 | 0.007502183 | 3242.712372 | 262 | 0.099993659 |
| 64 | 0.007187296 | 2053.131489 | 113 | 0.047726672 |
| 65 | 0.007014055 | 1839.627708 | 89 | 0.041057794 |
| 66 | 0.006838395 | 1797.933673 | 82 | 0.04106626 |
| 67 | 0.006392549 | 2550.07527 | 165 | 0.069740672 |
| 68 | 0.006348886 | 1194.412603 | 35 | 0.021855405 |
| 69 | 0.00600089 | 3339.709353 | 287 | 0.100842256 |
| 70 | 0.005957889 | 2643.521301 | 179 | 0.07280341 |
| 71 | 0.005951557 | 2528.041334 | 161 | 0.06935665 |
| 72 | 0.005948762 | 2539.648071 | 163 | 0.069601611 |
| 73 | 0.005946503 | 2289.180838 | 140 | 0.05645962 |
| 74 | 0.005912576 | 2251.709055 | 137 | 0.054458544 |
| 75 | 0.005531912 | 3177.329191 | 252 | 0.097030319 |
| 76 | 0.005034605 | 2707.443468 | 190 | 0.074477563 |
| 77 | 0.005023475 | 2827.501658 | 204 | 0.080520984 |
| 78 | 0.004672824 | 3324.060521 | 281 | 0.101375894 |
| 79 | 0.004650995 | 2605.038446 | 174 | 0.071196654 |
| 80 | 0.004054429 | 2772.676516 | 195 | 0.078761241 |
| 81 | 0.004021956 | 2050.285091 | 112 | 0.047917611 |
| 82 | 0.003977058 | 2692.859066 | 188 | 0.073882381 |
| 83 | 0.003901939 | 3816.059579 | 369 | 0.122607616 |
| 84 | 0.003894981 | 2988.023125 | 227 | 0.087406474 |
| 85 | 0.003857223 | 2781.252576 | 198 | 0.078395267 |
| 86 | 0.003420251 | 1421.889222 | 53 | 0.027297778 |
| 87 | 0.003390308 | 2434.427905 | 152 | 0.064443897 |
| 88 | 0.003377303 | 3316.092388 | 279 | 0.101278707 |
| 89 | 0.003359174 | 1610.458742 | 71 | 0.031965768 |
| 90 | 0.003329895 | 2796.081245 | 201 | 0.078648377 |
| 91 | 0.003273064 | 3261.962157 | 266 | 0.100481915 |
| 92 | 0.003005801 | 1906.49428 | 94 | 0.044136012 |
| 93 | 0.002940066 | 3296.630806 | 278 | 0.099642396 |
| 94 | 0.002558126 | 3025.599211 | 230 | 0.090021457 |
| 95 | 0.00194368 | 2992.736336 | 228 | 0.087492255 |
| 96 | 0.001663344 | 2295.859761 | 143 | 0.055840999 |
| 97 | 0.001647439 | 1821.743189 | 87 | 0.040565048 |
| 98 | 5.39744E−05 | 4046.661111 | 405 | 0.135705371 |
| 99 | 2.95539E−05 | 3107.068033 | 242 | 0.093653852 |
| 100 | 5.49626E−07 | 1940.201045 | 101 | 0.043712476 |

TABLE 10.b3

| | | | |
|---|---|---|---|
| 51 | 0.000192356 | −0.449176835 | −1.365261052 |
| 52 | 0.000191824 | −0.449219316 | −1.365301253 |
| 53 | 4.43478E−05 | −0.520758958 | −1.434709807 |
| 54 | 0.000111867 | −0.474511576 | −1.389447743 |
| 55 | 9.76587E−05 | −0.472816754 | −1.387816432 |
| 56 | 0.00013284 | −0.462833217 | −1.378245812 |
| 57 | 0.000109959 | −0.468441416 | −1.383613903 |
| 58 | 1.07885E−05 | −0.55536248 | −1.469537812 |
| 59 | 0.000204464 | −0.45184733 | −1.367790553 |
| 60 | 8.49952E−06 | −0.564485474 | −1.478859991 |
| 61 | 7.40111E−06 | −0.580204473 | −1.495061128 |
| 62 | 6.22175E−05 | −0.505076705 | −1.41919881 |
| 63 | 0.000185626 | −0.453123914 | −1.369001392 |
| 64 | 3.82124E−05 | −0.528006825 | −1.441935693 |
| 65 | 2.58911E−05 | −0.531999561 | −1.445931854 |
| 66 | 2.38597E−05 | −0.540379372 | −1.454354906 |
| 67 | 8.15334E−05 | −0.484410541 | −1.399014137 |
| 68 | 5.41991E−06 | −0.659803184 | −1.57986708 |
| 69 | 0.000205064 | −0.446723303 | −1.362941181 |
| 70 | 9.23358E−05 | −0.479857462 | −1.394605873 |
| 71 | 7.91187E−05 | −0.481407534 | −1.396105081 |
| 72 | 8.03845E−05 | −0.483910947 | −1.398529753 |
| 73 | 5.60056E−05 | −0.499967174 | −1.414181385 |
| 74 | 5.2863E−05 | −0.502676692 | −1.416839847 |
| 75 | 0.00017325 | −0.445141393 | −1.36144754 |
| 76 | 0.000100264 | −0.476969815 | −1.391817271 |
| 77 | 0.000116387 | −0.46082316 | −1.376326886 |
| 78 | 0.00020184 | −0.449207918 | −1.365290466 |
| 79 | 8.77757E−05 | −0.486416934 | −1.400961136 |
| 80 | 0.000108821 | −0.469997222 | −1.385106801 |
| 81 | 3.80258E−05 | −0.528321867 | −1.442250604 |
| 82 | 9.84156E−05 | −0.48526633 | −1.399844261 |
| 83 | 0.00032056 | −0.444816849 | −1.361141308 |
| 84 | 0.000140584 | −0.460873187 | −1.376374612 |
| 85 | 0.000109982 | −0.476574037 | −1.391435503 |
| 86 | 1.02511E−05 | −0.586479796 | −1.501578393 |
| 87 | 6.9405E−05 | −0.490494408 | −1.404926258 |
| 88 | 0.000200211 | −0.456776921 | −1.372472194 |
| 89 | 1.60808E−05 | −0.577246597 | −1.49199903 |
| 90 | 0.000112009 | −0.482269181 | −1.396939152 |
| 91 | 0.00018938 | −0.480626838 | −1.395349802 |
| 92 | 2.93959E−05 | −0.539584708 | −1.453554039 |
| 93 | 0.00019627 | −0.447705069 | −1.36386899 |
| 94 | 0.000146703 | −0.479164296 | −1.393935973 |
| 95 | 0.000141342 | −0.46100762 | −1.376502872 |
| 96 | 5.65789E−05 | −0.496397318 | −1.41068641 |
| 97 | 2.50056E−05 | −0.538371295 | −1.452332007 |
| 98 | 0.000389419 | −0.447100658 | −1.363297723 |
| 99 | 0.000160585 | −0.467352681 | −1.382570148 |
| 100 | 3.12818E−05 | −0.510132363 | −1.424180854 |

1. A process for detecting a chromosome state which represents a subgroup in a population comprising determining whether a chromosome interaction relating to that chromosome state is present or absent within a defined region of the genome; and
wherein the subgroup relates to the stage of fibrosis and the chromosome interaction corresponds to any one of the chromosome interactions represented by any probe shown in Table 1, 3 or 4; and/or
wherein the subgroup relates to the presence of fibrosis and the chromosome interaction corresponds to any one of the chromosome interactions represented by any probe shown in Table 2, 5 or 6.

2. The process according to claim 1 wherein the subgroup relates to the stage of sclerosis or the presence of sclerosis.

3. The process according to claim 1 wherein a specific combination of chromosome interactions are typed comprising at least 3, 5, 8, 10 or 20 chromosome interactions selected from all the chromosome interactions represented by the probes in all of Table 1, 3 or 4.

4. The process according to claim 1 wherein a specific combination of chromosome interactions are typed comprising at least 3, 5, 8, 10 or 20 chromosome interactions selected from all chromosome interactions represented by the probes in all of Table 2, 5 or 6.

5. The process according to claim 1 which is carried out to detect the whether the fibrosis is early stage or late stage fibrosis.

6. The process according to claim 1 which is carried out to determine the stage of fibrosis in an individual and wherein at least 10 chromosome interactions represented by the probes of any of Tables 1, 3 or 4 are typed.

7. The process according to claim 1 which is carried out to determine the stage of presence of fibrosis in an individual and wherein at least 10 chromosome interactions represented by the probes of any of Table 2, 5 or 6 are typed.

8. The process according to claim 1 in which the chromosome interactions are typed:
in a sample from an individual, and/or
by detecting the presence or absence of a DNA loop at the site of the chromosome interactions, and/or
detecting the presence or absence of distal regions of a chromosome being brought together in a chromosome conformation, and/or
by detecting the presence of a ligated nucleic acid which is generated during said typing and whose sequence comprises two regions each corresponding to the regions of the chromosome which come together in the chromosome interaction, wherein detection of the ligated nucleic acid is preferably by:
(i) by a probe that has at least 70% identity to any of the specific probe sequences mentioned in Table 1, 2, 3, 4, 5 or 6; and/or
(ii) by a primer pair which has at least 70% identity to any primer pair in Table 1, 2, 3, 4, 5 or 6.

9. The process according to claim 1, wherein said determining of whether the chromosome interaction is present or absent by a process comprising the steps of:
(i) cross-linking of chromosome regions which have come together in a chromosome interaction;
(ii) subjecting said cross-linked regions to cleavage, optionally by restriction digestion cleavage with an enzyme;
(iii) ligating said cross-linked cleaved DNA ends to form ligated DNA; and
(iv) detecting the presence or absence of the ligated nucleic acid to thereby determine presence or absence of the chromosome interaction.

10. The process according to claim 1 in which the presence or absence of at least the following markers is determined:
(a) the first 5, 8, 10 or 20 markers shown at the top of any of Tables 7, 8, 9 or 10; and/or
(b) 5 markers from the first 10 markers shown at the top of any of Tables 7, 8, 9 or 10; and/or
(c) 5 markers from the first 20, 30, 40 or 50 markers shown at the top of any of Tables 7, 8, 9 or 10; and/or
(d) 10 markers from the first 30, 40 or 50 markers shown at the top of any of Tables 7, 8, 9 or 10.

11. The process according to claim 1 in which the presence or absence of at least the following combinations of markers is determined:
(a) at least 5, 8, 10, 12, 15, 20 chromosome interactions selected from 1, 2, 3, 4, 5 of or all of the Tables 1, 2, 3, 4, 5 or 6; and/or
(b) at least 5, 8, 10, 12, 15, 20 chromosome interactions selected from all of Tables 3, 4, 5 and 6; and/or
(c) at least 3, 5, 8 or 10 chromosome interactions selected from each of Tables 3 and 4; and/or
(d) at least 3, 5, 8 or 10 chromosome interactions selected from each of Tables 5 and 6; and/or
(e) at least 10 chromosome interactions selected from each of Tables 3 and 4; and/or
(f) at least 10 chromosome interactions selected from each of Tables 5 and 6.

12. The process according to claim 1 wherein the presence or absence of at least one marker is determined from any one of Tables 1, 2, 3, 4, 5 or 6 and additionally the presence or absence of any number or combination of other chromosome interactions is determined.

13. The process according to claim 1 wherein:
the result of the process is provided in a report; and/or
the result of the process is used to select a treatment schedule; and/or
the result of the process is used to select a specific therapy for the individual; and/or
the process is carried out to select an individual for a medical treatment.

14. The process according to claim 1 which is carried out to identify or design a therapeutic agent for fibrosis;
wherein preferably said process is used to detect whether a candidate agent is able to cause a change to a chromosome state which is associated with fibrosis;
and wherein optionally:
the change in chromosomal interaction is monitored using (i) a probe that has at least 70% identity to any of the probe sequences mentioned in Table 1, 2, 3, 4, 5 or 6, and/or (ii) by a primer pair which has at least 70% identity to any primer pair in Table 1, 2, 3, 4, 5 or 6.

15. The process according to claim 14 which comprises selecting a target based on detection of the chromosome interactions, and preferably screening for a modulator of the target to identify a therapeutic agent for fibrosis, wherein said target is optionally a protein.

16. The process according to claim 1, wherein the typing or detecting comprises specific detection of the ligated product by quantitative PCR (qPCR) which uses primers capable of amplifying the ligated product and a probe which binds the ligation site during the PCR reaction, wherein said probe comprises sequence which is complementary to sequence from each of the chromosome regions that have come together in the chromosome interaction, wherein preferably said probe comprises:
an oligonucleotide which specifically binds to said ligated product, and/or
a fluorophore covalently attached to the 5′ end of the oligonucleotide, and/or
a quencher covalently attached to the 3′ end of the oligonucleotide, and
optionally
said fluorophore is selected from HEX, Texas Red and FAM; and/or
said probe comprises a nucleic acid sequence of length 10 to 40 nucleotide bases, preferably a length of 20 to 30 nucleotide bases.

17. The process according to claim 16 in which:
(a) the probe is the same as any probe listed in any of Tables 1, 2, 3, 4, 5 or 6, or has at least 70% identity with such a probe; and/or
(b) at least one primer is the same as any primer listed in any of Tables 1, 2, 3, 4, 5 or 6, or has at least 70% identity with such a primer; and/or
(c) both primers are the same as any pair of primers listed in any of Tables 1, 2, 3, 4, 5 or 6 or both primers have at least 70% identity with a such a primer pair.

18. A method of treating fibrosis in an individual that has been identified as being in need of the therapeutic agent by a process according to claim 1 comprising administering to said individual an agent which is therapeutic for fibrosis.


SEQUENCE LISTING (3401 sequences)

SEQ ID NO: 1 - DNA, 14 bp, Homo sapiens
  misc_feature (6)..(6): n is a, c, g, t or u
  misc_feature (9)..(9): n is a, c, g, t or u
ccgcgnggnggcag

SEQ ID NO: 2 - DNA, 60 bp, Homo sapiens
acaaaactgatgatgccttgcaacaaactcgatagaagagaaaaacaagctcagagcaac

SEQ ID NO: 3 - DNA, 60 bp, Homo sapiens
ccaacggcatataaaactgagaagagcttcgactctgagtcttcttcttcagtgttctgg

SEQ ID NO: 4 - DNA, 60 bp, Homo sapiens
tcctttccttcaagtctttaatcaaacctcgatgcagaacctgaatggacaacttctgga

SEQ ID NO: 5 - DNA, 60 bp, Homo sapiens
tgtaagactatcaaattagccaaaatcttcgaaagcagaaaataaaaagatagatttggt

SEQ ID NO: 6 - DNA, 60 bp, Homo sapiens
acaaaaattagaaaagtaactgataacctcgactacgaaaaataagtaagagtagcactt

SEQ ID NO: 7 - DNA, 60 bp, Homo sapiens
actgagggcaccgactatcagcaccgcgtcgaaaatcataatctggggccctctctcctc

SEQ ID NO: 8 - DNA, 60 bp, Homo sapiens
atatccaatcagagtggaaatatcagagtcgaaactaaaaaaaaaaaatccaattaaaaa

SEQ ID NO: 9 - DNA, 60 bp, Homo sapiens
gggtagggaagtaaaagaaaatggtgcttcgattcccattgaagctaagtctgcagttta

SEQ ID NO: 10 - DNA, 60 bp, Homo sapiens
ttcagttcaatgaggaaaagtctcataatcgaggatgaataaaacagacattgtcctcaa

SEQ ID NO: 11 - DNA, 60 bp, Homo sapiens
gaggtcagtaaggagagcagagtagacttcgattctcctcttatcactcccttccttgaa

SEQ ID NO: 12 - DNA, 60 bp, Homo sapiens
atgcggtgctgagttgtgctatggcaactcgaactcctaacctcaaatggtcttcccgcc

SEQ ID NO: 13 - DNA, 60 bp, Homo sapiens
agccatcaaacagaaccggaaaagtggctcgagaagagaatcgtaaatgaaatacataaa

SEQ ID NO: 14 - DNA, 60 bp, Homo sapiens
gggtagggaagtaaaagaaaatggtgcttcgatcctctccatcaagtctcctctctctag

SEQ ID NO: 15 - DNA, 60 bp, Homo sapiens
cctacaactttcttatttgtacttttcctcgataaatttatgtttatttaaaaaaatttg

SEQ ID NO: 16 - DNA, 60 bp, Homo sapiens
gaggtcagtaaggagagcagagtagacttcgaagaatatttaataaaatgtaaatatgcc

SEQ ID NO: 17 - DNA, 60 bp, Homo sapiens
gaggtcagtaaggagagcagagtagacttcgaatttctttattgacatgttctcactcat

SEQ ID NO: 18 - DNA, 60 bp, Homo sapiens
ccttctcttttcttccttttcctttctttcgatagatagagacatatatgagaggggatt

SEQ ID NO: 19 - DNA, 60 bp, Homo sapiens
gggtagggaagtaaaagaaaatggtgcttcgagttaagactgttctgagtcctgtaaaaa

SEQ ID NO: 20 - DNA, 60 bp, Homo sapiens
actgctttttgccataaagcctaccttatcgagaaaattcttaaaagaacactttttcct

SEQ ID NO: 21 - DNA, 60 bp, Homo sapiens
acataaaatgatatttcagattttgccgtcgagctatgtgagtctgagtaagtttcttaa

SEQ ID NO: 22 - DNA, 60 bp, Homo sapiens
gcggagatctttcaagaaatttatttaatcgatagtatgtacccttgatatgatatgatg

SEQ ID NO: 23 - DNA, 60 bp, Homo sapiens
acaaaaattagaaaagtaactgataacctcgagtcatctatttgatatctccacttcagt

SEQ ID NO: 24 - DNA, 60 bp, Homo sapiens
cactgagtattttcatctaatctttcactcgaatagttggaaaataattacctactcatc

SEQ ID NO: 25 - DNA, 60 bp, Homo sapiens
atggaaatctgagtaaagaaattcatggtcgagccaagaggaaggaagaaacggcggtgg

SEQ ID NO: 26 - DNA, 60 bp, Homo sapiens
cctggctccttcctgtattatgctcccttcgagaagtagaatatctgaatgttcttatat

SEQ ID NO: 27 - DNA, 60 bp, Homo sapiens
aaatacaaagaaaaacatcagagcagtatcgaccatgcactcagaagcttttacagggaa

SEQ ID NO: 28 - DNA, 60 bp, Homo sapiens
cacctcatcagcactcagaagagaacagtcgacagttaagagatcctggttaagggatgg

SEQ ID NO: 29 - DNA, 60 bp, Homo sapiens
tacaacaatttatttttggtttaaaagctcgagctcagctttccagcatgtgtaagctgt

SEQ ID NO: 30 - DNA, 60 bp, Homo sapiens
tggtctctacttaaaatacagaaattggtcgaaaatatatcagtttaagtgttattgagg

SEQ ID NO: 31 - DNA, 60 bp, Homo sapiens
acatacccctaagaatgctcagaaatactcgagaaatgatatgtttgtatactatccttt

SEQ ID NO: 32 - DNA, 60 bp, Homo sapiens
ctgttctaagcctaaaaagaatatatattcgactactgtgaatagggctgcaataagcat

SEQ ID NO: 33 - DNA, 60 bp, Homo sapiens
cctcacctccgccaatcagcaccgcgaatcgatccctttggattctcaggtccccatctt

SEQ ID NO: 34 - DNA, 60 bp, Homo sapiens
atatagaaatggtactgatttttgtacgtcgaaattaaaagttcaaaaatacatttctta

SEQ ID NO: 35 - DNA, 60 bp, Homo sapiens
ggggaagataagtgatcatatttctacttcgatcaagcatacataatagacatctacaga

SEQ ID NO: 36 - DNA, 60 bp, Homo sapiens
agtctaaaatagtggtttgaacaagtagtcgaggaggaaagaaaaacctctcaggtccat

SEQ ID NO: 37 - DNA, 60 bp, Homo sapiens
atgcaagagaaattacataacagagggctcgatgtttaccttgaactgagtaaaatcaaa

SEQ ID NO: 38 - DNA, 60 bp, Homo sapiens
agccatggacaacttattgtgttcaagttcgatttgttacagacgttaacaaattccttt

SEQ ID NO: 39 - DNA, 60 bp, Homo sapiens
atgcttattgcagccctattcacagtagtcgaattattttcaaataacatcataaaattc

SEQ ID NO: 40 - DNA, 60 bp, Homo sapiens
tccctgtcttctaaataataagagagaatcgacactagacttgtttagaagacctcactg

SEQ ID NO: 41 - DNA, 60 bp, Homo sapiens
gatatgcagctactatcttactgcatcctcgatattaaatccctactaacttccaatgat

SEQ ID NO: 42 - DNA, 60 bp, Homo sapiens
atgcaagagaaattacataacagagggctcgactcatgagtctgactgctttgtgcctta

SEQ ID NO: 43 - DNA, 60 bp, Homo sapiens
agctgacttatgtaccagatactttgtttcgatcagggtagactgagaagtgaatgtgaa

SEQ ID NO: 44 - DNA, 60 bp, Homo sapiens
gcagatctctagaacttaatttgcaaattcgattaaatgcttattatgtgtttatttagc

SEQ ID NO: 45 - DNA, 60 bp, Homo sapiens
taacattctgataccactacacctgagttcgactaattatgatggtgaaggagcagaaga

SEQ ID NO: 46 - DNA, 60 bp, Homo sapiens
tctagagagaacatctttcttcagcagatcgaagtgttctttctctgtatttgtctcgtt

SEQ ID NO: 47 - DNA, 60 bp, Homo sapiens
cattggaggcactcaaaaaaaaaaaaaatcgattgtgacctaatttgataacagggcttt

SEQ ID NO: 48 - DNA, 60 bp, Homo sapiens
aaattatattttcttaggttaatcgtcctcgatcattatatttaaggcccacactaattc

SEQ ID NO: 49 - DNA, 60 bp, Homo sapiens
acctgctgggacaactgaaagattattatcgagtctaaacatctacatgggattagtaat

SEQ ID NO: 50 - DNA, 60 bp, Homo sapiens
cctacaactttcttatttgtacttttcctcgaggaaataattcactaggtaaagggagta

SEQ ID NO: 51 - DNA, 60 bp, Homo sapiens
tacagatatgcttgttgtatgtcacgtgtcgacataaatatttgttgaacaagtaaatga

SEQ ID NO: 52 - DNA, 23 bp, Homo sapiens
catctgacctcctccagcctctg

SEQ ID NO: 53 - DNA, 23 bp, Homo sapiens
ctctgaagccacacccacacttg

SEQ ID NO: 54 - DNA, 26 bp, Homo sapiens
cttggagtcccagtggaaaaggttga

SEQ ID NO: 55 - DNA, 26 bp, Homo sapiens
ccaagtgtattgttagcaccagcggt

SEQ ID NO: 56 - DNA, 23 bp, Homo sapiens
atgctctgccccactgtccttgc

SEQ ID NO: 57 - DNA, 23 bp, Homo sapiens
gtgatttcagtgggtctccaggg

SEQ ID NO: 58 - DNA, 26 bp, Homo sapiens
ccatactacctttaggaggaacttgc

SEQ ID NO: 59 - DNA, 26 bp, Homo sapiens
tggagaaaacgcaaacgcctacaggg

SEQ ID NO: 60 - DNA, 26 bp, Homo sapiens
cagtaaagggaaccaaagaaaacaac

SEQ ID NO: 61 - DNA, 26 bp, Homo sapiens
caacttccacctttgaatccatttct

SEQ ID NO: 62 - DNA, 23 bp, Homo sapiens
ggctgctcctcacctggcttggt

SEQ ID NO: 63 - DNA, 23 bp, Homo sapiens
tcccaaccccttccagaatcttc

SEQ ID NO: 64 - DNA, 23 bp, Homo sapiens
gcaatgctaacagccttgacagc

SEQ ID NO: 65 - DNA, 26 bp, Homo sapiens
ttttcttcgtgctggtgctggtgagc

SEQ ID NO: 66 - DNA, 24 bp, Homo sapiens
cctggaaggatgtcagatgagtct

SEQ ID NO: 67 - DNA, 24 bp, Homo sapiens
aagcaatgatagttggcgatacat

SEQ ID NO: 68 - DNA, 26 bp, Homo sapiens
gggtgaggtagaagtatgaaggaaag

SEQ ID NO: 69 - DNA, 26 bp, Homo sapiens
ccttcctcccttgtctgcctgtagat

SEQ ID NO: 70 - DNA, 26 bp, Homo sapiens
cacaggattggaatgaggaataaaga

SEQ ID NO: 71 - DNA, 23 bp, Homo sapiens
ccaatgagggttccagggttcaa

SEQ ID NO: 72 - DNA, 23 bp, Homo sapiens
tccttcctacaacaccctgccag

SEQ ID NO: 73 - DNA, 23 bp, Homo sapiens
agtggcaggtaacggctgggcat

SEQ ID NO: 74 - DNA, 26 bp, Homo sapiens
tgttgaatgaatgagcaggagccagc

SEQ ID NO: 75 - DNA, 26 bp, Homo sapiens
cacagccagtaaacagcattcaaggg

SEQ ID NO: 76 - DNA, 23 bp, Homo sapiens
ggctctgctgggacttgggactt

SEQ ID NO: 77 - DNA, 23 bp, Homo sapiens
agagtggcttgaccagagtgtgg

SEQ ID NO: 78 - DNA, 26 bp, Homo sapiens
gaagagtgcttggcagagtgtgtgct

SEQ ID NO: 79 - DNA, 26 bp, Homo sapiens
ttgttcctggtttacagcccctacta

SEQ ID NO: 80 - DNA, 26 bp, Homo sapiens
ctacacacaggattggaatgaggaat

SEQ ID NO: 81 - DNA, 26 bp, Homo sapiens
gcacacagcgaaataatagttgttag

SEQ ID NO: 82 - DNA, 26 bp, Homo sapiens
cacaggattggaatgaggaataaaga

SEQ ID NO: 83 - DNA, 23 bp, Homo sapiens
ccacagtgtttccatcagcctgg

SEQ ID NO: 84 - DNA, 26 bp, Homo sapiens
ctccactgctgaaacaatagcgacgc

SEQ ID NO: 85 - DNA, 26 bp, Homo sapiens
cataagccaactcccctaatgaatcc

SEQ ID NO: 86 - DNA, 26 bp, Homo sapiens
agatgagtctgcccattgctcctgag

SEQ ID NO: 87 - DNA, 26 bp, Homo sapiens
ctcagaagtcagttcatccatttcct

SEQ ID NO: 88 - DNA, 26 bp, Homo sapiens
cctcctacagtgattgtggatttgtc

SEQ ID NO: 89 - DNA, 26 bp, Homo sapiens
gggtaggaatgtgagtgaagcaggac

SEQ ID NO: 90 - DNA, 23 bp, Homo sapiens
catcctgtccagattaggcagcc

SEQ ID NO: 91 - DNA, 26 bp, Homo sapiens
ccagatactgtgctaaaggcttcgcc

SEQ ID NO: 92 - DNA, 24 bp, Homo sapiens
cagggagaaggagaaggagatttc

SEQ ID NO: 93 - DNA, 24 bp, Homo sapiens
tgaccttgacagttttgaggagta

SEQ ID NO: 94 - DNA, 26 bp, Homo sapiens
gcttccagccaacatttgagtgcctc

SEQ ID NO: 95 - DNA, 26 bp, Homo sapiens
ccaggtatggaaaaggagattgtgtc

SEQ ID NO: 96 - DNA, 26 bp, Homo sapiens
cctggtcattggtgaaggtctggcat

SEQ ID NO: 97 - DNA, 26 bp, Homo sapiens
gacacccaaccataatgacagagatt

SEQ ID NO: 98 - DNA, 26 bp, Homo sapiens
ggatgtgtgagagaaagggcattggc

SEQ ID NO: 99 - DNA, 26 bp, Homo sapiens
tttacactttcgccttctcttccagc

SEQ ID NO: 100 - DNA, 24 bp, Homo sapiens
tggaggcatctctgacaccaatca

SEQ ID NO: 101 - DNA, 24 bp, Homo sapiens
gaaagaatacactgatgaagccat

SEQ ID NO: 102 - DNA, 23 bp, Homo sapiens
acctcctgggatgtctcagtggc

SEQ ID NO: 103 - DNA, 23 bp, Homo sapiens
gaggcagatgtgggaggtttgct

SEQ ID NO: 104 - DNA, 26 bp, Homo sapiens
gctgggtatggctggtttgtgtttta

SEQ ID NO: 105 - DNA, 26 bp, Homo sapiens
cctgctttccataaacctctgcttct

SEQ ID NO: 106 - DNA, 26 bp, Homo sapiens
agaggttcatcgtctccaggtcatag

SEQ ID NO: 107 - DNA, 26 bp, Homo sapiens
ttgttgggtgtgatgtcctccacttc

SEQ ID NO: 108 - DNA, 23 bp, Homo sapiens
gaccaggagttcaagaccagcct

SEQ ID NO: 109 - DNA, 23 bp, Homo sapiens
atgtaaccctgagagaagacctc

SEQ ID NO: 110 - DNA, 23 bp, Homo sapiens
ggcaagggcttatttaggagcat

SEQ ID NO: 111 - DNA, 26 bp, Homo sapiens
gattcacaggaagaaaactggaacta

SEQ ID NO: 112 - DNA, 26 bp, Homo sapiens
gcatagatgggttagtcagagaccac

SEQ ID NO: 113 - DNA, 26 bp, Homo sapiens
ggtctcacaatcacactgctgggaat

SEQ ID NO: 114 - DNA, 23 bp, Homo sapiens
tgggtgaggaggtggagcagccg

SEQ ID NO: 115 - DNA, 23 bp, Homo sapiens
aaatacgccaggacacaggcagc

SEQ ID NO: 116 - DNA, 26 bp, Homo sapiens
gtagtcttgagttggctccctgtttg

SEQ ID NO: 117 - DNA, 26 bp, Homo sapiens
caactcgctccactttggcatcgtat

SEQ ID NO: 118 - DNA, 26 bp, Homo sapiens
gcagtagatttctgttgaggtgatta

SEQ ID NO: 119 - DNA, 26 bp, Homo sapiens
cagttttagaataagtgctatgtggc

SEQ ID NO: 120 - DNA, 23 bp, Homo sapiens
ctacttccctttccatcccaccc

SEQ ID NO: 121 - DNA, 23 bp, Homo sapiens
ccccaaacattctgccagggtcc

SEQ ID NO: 122 - DNA, 24 bp, Homo sapiens
gggtctaaggaaggtcaaggctgc

SEQ ID NO: 123 - DNA, 23 bp, Homo sapiens
gcccaagcccctattagacccat

SEQ ID NO: 124 - DNA, 26 bp, Homo sapiens
gcacctgctacctgctccttcctatt

SEQ ID NO: 125 - DNA, 26 bp, Homo sapiens
aactgcggaaaggtcatagcataacc

SEQ ID NO: 126 - DNA, 23 bp, Homo sapiens
ggtctcacaatcacactgctggg

SEQ ID NO: 127 - DNA, 23 bp, Homo sapiens
cgacacttccccatctccccaac

SEQ ID NO: 128 - DNA, 26 bp, Homo sapiens
ttttcaaatccctcttgtgccctcat

SEQ ID NO: 129 - DNA, 26 bp, Homo sapiens
ccacaatcatctccctttccctctga

SEQ ID NO: 130 - DNA, 26 bp, Homo sapiens
tccttcctttcctactcagtgtggat

SEQ ID NO: 131 - DNA, 26 bp, Homo sapiens
agtctacctgaggaggcaacagccat

SEQ ID NO: 132 - DNA, 26 bp, Homo sapiens
cactaaagcactccagtcaagtcagt

SEQ ID NO: 133 - DNA, 26 bp, Homo sapiens
ggatggcgaaataccacgggaagcat

SEQ ID NO: 134 - DNA, 26 bp, Homo sapiens
gggtagggagaggaaggaaacggata

SEQ ID NO: 135 - DNA, 26 bp, Homo sapiens
caatagtgcttgttcctacctctcac

SEQ ID NO: 136 - DNA, 24 bp, Homo sapiens
gattctgtagtcatcaggaacacg

SEQ ID NO: 137 - DNA, 23 bp, Homo sapiens
caggagagtgtgagagagcccga

SEQ ID NO: 138 - DNA, 26 bp, Homo sapiens
gtctgctttggaacatcaagagtgct

SEQ ID NO: 139 - DNA, 26 bp, Homo sapiens
gctgacctcctcatcttcacccacaa

SEQ ID NO: 140 - DNA, 26 bp, Homo sapiens
tgatttctgcccagccgagtgctaca

SEQ ID NO: 141 - DNA, 26 bp, Homo sapiens
atacattacctttctgctcagccagc

SEQ ID NO: 142 - DNA, 24 bp, Homo sapiens
ggaccattgagagaataacagata

SEQ ID NO: 143 - DNA, 25 bp, Homo sapiens
gaaagagaatcacaaagcataaata

SEQ ID NO: 144 - DNA, 23 bp, Homo sapiens
gagatggatggtggcaatggttg

SEQ ID NO: 145 - DNA, 26 bp, Homo sapiens
atcttgttgggaaataaggtctttgc

SEQ ID NO: 146 - DNA, 24 bp, Homo sapiens
aacacagtggctcttctctttgaa

SEQ ID NO: 147 - DNA, 24 bp, Homo sapiens
ccctgtctcaaaacaaaacaaata

SEQ ID NO: 148 - DNA, 23 bp, Homo sapiens
gagtgcttggcagagtgtgtgct

SEQ ID NO: 149 - DNA, 23 bp, Homo sapiens
ggtaggtgatggtctgcccttcc

SEQ ID NO: 150 - DNA, 26 bp, Homo sapiens
ccttgccttggcttctctcgtaaaga

SEQ ID NO: 151 - DNA, 26 bp, Homo sapiens
caacagccgcacactcattcactcat

SEQ ID NO: 152 - DNA, 60 bp, Homo sapiens
aaaaaacaattatgtaattgaaaacccatcgaagtacaaaagtacccaaaggctttcaga

SEQ ID NO: 153 - DNA, 60 bp, Homo sapiens
aagaaagtaccagaaaggttattattaatcgatttacaaaacttgatggttagtaatttt

SEQ ID NO: 154 - DNA, 60 bp, Homo sapiens
ctggttgacaataactatacaaagagtgtcgaaatatgttcctaaaatattcaaagtatt

SEQ ID NO: 155 - DNA, 60 bp, Homo sapiens
ctggttgacaataactatacaaagagtgtcgacagagcttaacagctgtttcaaagtctt

SEQ ID NO: 156 - DNA, 60 bp, Homo sapiens
ccagacacaggtagttcctaaaatcttctcgaaggcttttatttaatttgagaatcatca

SEQ ID NO: 157 - DNA, 60 bp, Homo sapiens
gaacccaggagacggagcttgcagtgagtcgagtattatttagtcataaaaaggaatgaa

SEQ ID NO: 158 - DNA, 60 bp, Homo sapiens
gtcttctatcaaatcagattttattttctcgaatcagttcttgattctaatctccaatgt

SEQ ID NO: 159 - DNA, 60 bp, Homo sapiens
gtagtataataataaactacctgttgattcgatttaattattagttattgttgttaatct

SEQ ID NO: 160 - DNA, 60 bp, Homo sapiens
aatgacataattggcctcaaaacatatttcgagagttggctagattcatacacataatta

SEQ ID NO: 161 - DNA, 60 bp, Homo sapiens
tcctctttttttatatttttttattttttcgaaaaatgaataaattaattaattaaataa

SEQ ID NO: 162 - DNA, 60 bp, Homo sapiens
ggatctagaaaagagtattttcaatgtgtcgaatgtgttgcaaatatttttctaagctta

SEQ ID NO: 163 - DNA, 60 bp, Homo sapiens
gaggcgggtggatcatgaggtcggaagatcgatatgcaggctggaacccgccaaacattt

SEQ ID NO: 164 - DNA, 60 bp, Homo sapiens
aataaagcacacaataaatgtaatgggctcgacattttgaatatgtgggatccaaatctg

SEQ ID NO: 165 - DNA, 60 bp, Homo sapiens
taaaaaagatcggataattaggaaattatcgatgataaaatatatgtaccccacttagag

SEQ ID NO: 166 - DNA, 60 bp, Homo sapiens
ccctaaagtctgggaaggaaagagaggctcgattcctgttttcccttctgcctccctcgt

SEQ ID NO: 167 - DNA, 60 bp, Homo sapiens
cagcgtacatttctgaacattgtgaacgtcgagtcagaaaagatactcaataagattttg

SEQ ID NO: 168 - DNA, 60 bp, Homo sapiens
gggtttcaccatgttggtcaggctggtctcgaggccagcctgaccaacatgaagaaaccc

SEQ ID NO: 169 - DNA, 60 bp, Homo sapiens
gagaatctgctcagagctgatttcgttatcgagcagtaggaactaggcgtgtgtagattc

SEQ ID NO: 170 - DNA, 60 bp, Homo sapiens
agtttgcctatattggaatcattagcattcgaattgtgaaacaaaaactgtttgctgggc

SEQ ID NO: 171 - DNA, 60 bp, Homo sapiens
gttacttgtctgcagttactttcttgaatcgaggcagcagacaaggttctgatcttggct

SEQ ID NO: 172 - DNA, 60 bp, Homo sapiens
tggtgcatttatgatatacactgtgttatcgaaaaaaattaagtaataataataatagag

SEQ ID NO: 173 - DNA, 60 bp, Homo sapiens
ggacataactgtattctctctctctctctcgatgagcttaaagtggtgttctaggaccag

SEQ ID NO: 174 - DNA, 60 bp, Homo sapiens
aatatgtataaattacaaaatggctaaatcgatatctgccctaacaagaatccttctagt

SEQ ID NO: 175 - DNA, 60 bp, Homo sapiens
attctgaagaaatcaggaaataatgcattcgatagataatagatagaaatatgcagaaag

SEQ ID NO: 176 - DNA, 60 bp, Homo sapiens
atagtatattatctaaaatgtcaaattttcgaaactaaaaaaaaaaaatccaattaaaaa

SEQ ID NO: 177 - DNA, 60 bp, Homo sapiens
gtcgtgcagtggtgatgatctcctgcaatcgaagaacaaagagatgaaaagagctttcta

SEQ ID NO: 178 - DNA, 60 bp, Homo sapiens
gctggaaaatcaggctcatcacaaaagctcgagacccccacaaagaggaagtccacagca

SEQ ID NO: 179 - DNA, 60 bp, Homo sapiens
cctcagtctttctttgttttccaagacctcgaggtatttttcctaatgaaaaacaaagaa

SEQ ID NO: 180 - DNA, 60 bp, Homo sapiens
ctgcaatgttcaactaagctctgtccaatcgaggttgttagttttgaagattgaggaagg

SEQ ID NO: 181 - DNA, 60 bp, Homo sapiens
aaaaatgtgtatttctacatttttttggtcgaatgaatagaatgacaataggtttttatt

SEQ ID NO: 182 - DNA, 60 bp, Homo sapiens
aacatagcatattcaactaagcaattcatcgaagtatccttatattttagattaacatat

SEQ ID NO: 183 - DNA, 60 bp, Homo sapiens
ggctcggcgggtacccggcctagtgagctcgagaaactgtcttaacatttttatattcaa

SEQ ID NO: 184 - DNA, 60 bp, Homo sapiens
agctctggcagttttctgaaataccatatcgagctgtggtgattactttctccctgaaac

SEQ ID NO: 185 - DNA, 60 bp, Homo sapiens
tcactagcagattcctgcattctttcagtcgaagagtgaaccttgatgtatgcagatttt

SEQ ID NO: 186 - DNA, 60 bp, Homo sapiens
atgtcttaatattattataatcattgtttcgatatgccaagttccctgtctgcctacagt

SEQ ID NO: 187 - DNA, 60 bp, Homo sapiens
ggtcgttgaggacaatttacaaattggctcgatggtgtaaatattcctggaaggccaagg

SEQ ID NO: 188 - DNA, 60 bp, Homo sapiens
cactttttacacatgagaggtgaggatatcgaccaagagatcgcgagatgggagcagaga

SEQ ID NO: 189 - DNA, 60 bp, Homo sapiens
aacaactgctgcaagaggggccgtttgttcgatgctacttttaatcccagaacaagtatt

SEQ ID NO: 190 - DNA, 60 bp, Homo sapiens
gggtttcaccatgttggccagactgatctcgagacgatcccggccaacatggtgaaaccc

SEQ ID NO: 191 - DNA, 60 bp, Homo sapiens
ctgcagcccgtagttgtggatgatggagtcgattttttcccttcttcttgcatttggaac

SEQ ID NO: 192 - DNA, 60 bp, Homo sapiens
gagagagagagagaaagagacgaagatttcgaccactcttgagctgtgtgatgttgatct

SEQ ID NO: 193 - DNA, 60 bp, Homo sapiens
ggacataactgtattctctctctctctctcgaggaaggttctgaggaattgaagcccact

SEQ ID NO: 194 - DNA, 60 bp, Homo sapiens
ggcaggtggctcacctgaggtcgggagttcgaagtctgtttcagatgaagttttaagatc

SEQ ID NO: 195 - DNA, 60 bp, Homo sapiens
acggagtggaaggagctgcaactttccatcgagttcgccctccctgacagggagttgtaa

SEQ ID NO: 196 - DNA, 60 bp, Homo sapiens
aagttagatgcaaaggcctgactgtgtttcgagcatagatcccagccccagatcccacag

SEQ ID NO: 197 - DNA, 60 bp, Homo sapiens
tccagctatctgctcagggaattgttattcgagccgcccccgaggcttggagtcgggctg

SEQ ID NO: 198 - DNA, 60 bp, Homo sapiens
aggagtggccaggcagagccaagtcctatcgagtggctctgtcacttattagcaagtcac

SEQ ID NO: 199 - DNA, 60 bp, Homo sapiens
aatctgtctctctcttactccctctctctcgaaaatactgtactttggttgatttttaag

SEQ ID NO: 200 - DNA, 60 bp, Homo sapiens
aatctgtctctctcttactccctctctctcgatgggtatggaagcaaaggcctgaagaat

SEQ ID NO: 201 - DNA, 60 bp, Homo sapiens
ggatctagaaaagagtattttcaatgtgtcgacacagatgattagggcatcttctgctct

SEQ ID NO: 202 - DNA, 23 bp, Homo sapiens
ccagccaatcacaagcagcctac

SEQ ID NO: 203 - DNA, 23 bp, Homo sapiens
ctccttcagcacccaccccaaca

SEQ ID NO: 204 - DNA, 26 bp, Homo sapiens
ctttcatcctttattgtattggtgcg

SEQ ID NO: 205 - DNA, 23 bp, Homo sapiens
ggattacaggcgtgagccaacat

SEQ ID NO: 206 - DNA, 23 bp, Homo sapiens
tcctcactctcctgccactccct

SEQ ID NO: 207 - DNA, 23 bp, Homo sapiens
ccaagtaagtggaggctggctct

SEQ ID NO: 208 - DNA, 23 bp, Homo sapiens
ctcactctcctgccactccctgc

SEQ ID NO: 209 - DNA, 26 bp, Homo sapiens
caagaaaatccagatgctcaaaacac

SEQ ID NO: 210 - DNA, 23 bp, Homo sapiens
tgtggacagatgctgagaccctc

SEQ ID NO: 211 - DNA, 24 bp, Homo sapiens
gagacagggcttcagagtcacagg

SEQ ID NO: 212 - DNA, 23 bp, Homo sapiens
tactcgggaggctgaggcagaat

SEQ ID NO: 213 - DNA, 26 bp, Homo sapiens
ctctgtggatttacctattttggaca

SEQ ID NO: 214 - DNA, 26 bp, Homo sapiens
ggaggaagtggacagtaagttgatac

SEQ ID NO: 215 - DNA, 26 bp, Homo sapiens
atggacaggcagaggtggtgacaatg

SEQ ID NO: 216 - DNA, 26 bp, Homo sapiens
ccatccaccctgcctttcataagaga

SEQ ID NO: 217 - DNA, 26 bp, Homo sapiens
ggcaggaaagtggcttaggaccaaaa

SEQ ID NO: 218 - DNA, 24 bp, Homo sapiens
agtgagactactgaccaaagaatc

SEQ ID NO: 219 - DNA, 24 bp, Homo sapiens
gctgaaaccccaagataaaactat

SEQ ID NO: 220 - DNA, 23 bp, Homo sapiens
gggtggtgagggtagagacttac

SEQ ID NO: 221 - DNA, 23 bp, Homo sapiens
cccagggcttcaactccccatct

SEQ ID NO: 222 - DNA, 24 bp, Homo sapiens
caatgggctctgaaatgtcacttc

SEQ ID NO: 223 - DNA, 26 bp, Homo sapiens
catcaaaatgtcgtgtggtgtatcaa

SEQ ID NO: 224 - DNA, 26 bp, Homo sapiens
ggaggtatgagaaggctattgtagga

SEQ ID NO: 225 - DNA, 26 bp, Homo sapiens
tcagaggaaggctggagtgaatccct

SEQ ID NO: 226 - DNA, 26 bp, Homo sapiens
ctgattctatgttatggtgagttgtg

SEQ ID NO: 227 - DNA, 24 bp, Homo sapiens
ccatcatcggttgaatccacagat

SEQ ID NO: 228 - DNA, 23 bp, Homo sapiens
gcctgggtgacagagcaagactc

SEQ ID NO: 229 - DNA, 23 bp, Homo sapiens
aaccttagccccacaatcagagc

SEQ ID NO: 230 - DNA, 23 bp, Homo sapiens
aggattggaactgttagccccat

SEQ ID NO: 231 - DNA, 23 bp, Homo sapiens
ctctgatggctgtgctgaggaca

SEQ ID NO: 232 - DNA, 26 bp, Homo sapiens
gcctgccctgcggtttcaaagtagat

SEQ ID NO: 233 - DNA, 26 bp, Homo sapiens
gcaaaacacacattcttctcatcggc

SEQ ID NO: 234 - DNA, 23 bp, Homo sapiens
agccttccgagtaggtgggagta

SEQ ID NO: 235 - DNA, 23 bp, Homo sapiens
actctcctgcctcagcctcttga

SEQ ID NO: 236 - DNA, 23 bp, Homo sapiens
ttttctggcagcctctccttccg

SEQ ID NO: 237 - DNA, 23 bp, Homo sapiens
aaccctcagccaggagccaggat

SEQ ID NO: 238 - DNA, 26 bp, Homo sapiens
gtgccagaacacgagagacacatagg

SEQ ID NO: 239 - DNA, 26 bp, Homo sapiens
aaactccctaagtccgtaaatacccc

SEQ ID NO: 240 - DNA, 23 bp, Homo sapiens
aggacagagcagaggtgggactc

SEQ ID NO: 241 - DNA, 23 bp, Homo sapiens
tcacagggctcagcagtggagaa

SEQ ID NO: 242 - DNA, 26 bp, Homo sapiens
tgtcctgttgctcatagaagttgcta

SEQ ID NO: 243 - DNA, 26 bp, Homo sapiens
cacggtaatgatactaatgaaggcta

SEQ ID NO: 244 - DNA, 23 bp, Homo sapiens
ggataaagcacaggatgccccag

SEQ ID NO: 245 - DNA, 23 bp, Homo sapiens
cacactccaccactcagagcagg

SEQ ID NO: 246 - DNA, 26 bp, Homo sapiens
ccaaatggtctggttctgggaatctt

SEQ ID NO: 247 - DNA, 26 bp, Homo sapiens
gaagcctgtttggacaagggacacct

SEQ ID NO: 248 - DNA, 23 bp, Homo sapiens
gatgctgtccctttagtggtcaa

SEQ ID NO: 249 - DNA, 26 bp, Homo sapiens
cactttgtgtcctgttcataactttc

SEQ ID NO: 250 - DNA, 26 bp, Homo sapiens
gggcttcagggaaaatcagtattctg

SEQ ID NO: 251 - DNA, 26 bp, Homo sapiens
ttttcttcgtgctggtgctggtgagc

SEQ ID NO: 252 - DNA, 26 bp, Homo sapiens
cattttccttgtccctcctcaggtaa

SEQ ID NO: 253 - DNA, 26 bp, Homo sapiens
tgccgtgattcttctccctcctttgc

SEQ ID NO: 254 - DNA, 23 bp, Homo sapiens
caggctggtgggaaggagacatt

SEQ ID NO: 255 - DNA, 23 bp, Homo sapiens
tggcagagggacagactccagtg

SEQ ID NO: 256 - DNA, 26 bp, Homo sapiens
tgtctctttagtcccctgtggtctgt

SEQ ID NO: 257 - DNA, 26 bp, Homo sapiens
tgtttccttttgtctcctccactggc

SEQ ID NO: 258 - DNA, 26 bp, Homo sapiens
tagtgtcctcccagtgctgtagtgag

SEQ ID NO: 259 - DNA, 26 bp, Homo sapiens
tcagggagaacgcttttccttgattc

SEQ ID NO: 260 - DNA, 24 bp, Homo sapiens
tatttggctgaaaagcactcttgc

SEQ ID NO: 261 - DNA, 24 bp, Homo sapiens
actacttctactcaactcatccat

SEQ ID NO: 262 - DNA, 26 bp, Homo sapiens
ggcgatggacacccttgtaacctttc

SEQ ID NO: 263 - DNA, 26 bp, Homo sapiens
ccccacactttcagatgtcagtagta

SEQ ID NO: 264 - DNA, 23 bp, Homo sapiens
gctgggctcgctgattggctgcg

SEQ ID NO: 265 - DNA, 23 bp, Homo sapiens
gcccctgaaaagagccatttgaa

SEQ ID NO: 266 - DNA, 23 bp, Homo sapiens
ggaatagagataacgggctgggc

SEQ ID NO: 267 - DNA, 23 bp, Homo sapiens
gggtgggtgacaaaggagagagg

SEQ ID NO: 268 - DNA, 23 bp, Homo sapiens
gccaagggtcggttgagtcacag

SEQ ID NO: 269 - DNA, 23 bp, Homo sapiens
gtagtctgcgttccaccttggga

SEQ ID NO: 270 - DNA, 26 bp, Homo sapiens
gaaacatagtgagaagacaggcattg

SEQ ID NO: 271 - DNA, 26 bp, Homo sapiens
ctccagatgttcttccttccacactg

SEQ ID NO: 272 - DNA, 26 bp, Homo sapiens
caccagttaccaccaacatacattat

SEQ ID NO: 273 - DNA, 23 bp, Homo sapiens
ggtcaggctggtcttgaactcct

SEQ ID NO: 274 - DNA, 23 bp, Homo sapiens
gaccgattgctcctctttagccc

SEQ ID NO: 275 - DNA, 23 bp, Homo sapiens
tcccctcatcactgcggtcccta

SEQ ID NO: 276 - DNA, 26 bp, Homo sapiens
gggatttggacgagagacacaggatg

SEQ ID NO: 277 - DNA, 26 bp, Homo sapiens
cggtttgaacagagttgtggatagca

SEQ ID NO: 278 - DNA, 23 bp, Homo sapiens
cctcccaggttcaatcaggcaat

SEQ ID NO: 279 - DNA, 23 bp, Homo sapiens
ctcctacctcatcctcccgagta

SEQ ID NO: 280 - DNA, 23 bp, Homo sapiens
caggaactcatcgcccgtgcggc

SEQ ID NO: 281 - DNA, 23 bp, Homo sapiens
ttctcttgctggacaactgttcc

SEQ ID NO: 282 - DNA, 23 bp, Homo sapiens
gagcgagagcaggagtgagtgag

SEQ ID NO: 283 - DNA, 23 bp, Homo sapiens
agacaggctgagaggtttggctg

SEQ ID NO: 284 - DNA, 23 bp, Homo sapiens
ggataaagcacaggatgccccag

SEQ ID NO: 285 - DNA, 23 bp, Homo sapiens
ctccaccttccccactcaagcag

SEQ ID NO: 286 - DNA, 23 bp, Homo sapiens
tgcctgtaatcccagcactttgg

SEQ ID NO: 287 - DNA, 26 bp, Homo sapiens
caccactgcggttttacatttatgat

SEQ ID NO: 288 - DNA, 26 bp, Homo sapiens
aagaaacaaagcgaggcaaggtgacg

SEQ ID NO: 289 - DNA, 26 bp, Homo sapiens
aggcagacttctttggagggtgtcaa

SEQ ID NO: 290 - DNA, 24 bp, Homo sapiens
gtggttacaacataccccttcccc

SEQ ID NO: 291 - DNA, 23 bp, Homo sapiens
cctgcgggacaaatccaacccac

SEQ ID NO: 292 - DNA, 23 bp, Homo sapiens
gcgaagtgtccagcatcgtcctg

SEQ ID NO: 293 - DNA, 23 bp, Homo sapiens
agtggacgaatgaaaggcacccc

SEQ ID NO: 294 - DNA, 23 bp, Homo sapiens
caagacagaggcaagcccaggaa

SEQ ID NO: 295 - DNA, 23 bp, Homo sapiens
tcccagaaccctgctgcctccat

SEQ ID NO: 296 - DNA, 23 bp, Homo sapiens
gcctccaaaagtgtatgagccaa

SEQ ID NO: 297 - DNA, 24 bp, Homo sapiens
caacagttcttcagcacccaagta

SEQ ID NO: 298 - DNA, 23 bp, Homo sapiens
gcctccaaaagtgtatgagccaa

SEQ ID NO: 299 - DNA, 23 bp, Homo sapiens
tgcttcccctcctcaccactcta

SEQ ID NO: 300 - DNA, 26 bp, Homo sapiens
ccgcatcgcaaaggattttctcaaag

SEQ ID NO: 301 - DNA, 26 bp, Homo sapiens
gaaagtgggatgggagaacagagcca

SEQ ID NO: 302 - DNA, 60 bp, Homo sapiens
caatcaaaatgttgcagtatctttttaatcgagatttaaccacatctgatttcatgggtt

SEQ ID NO: 303 - DNA, 60 bp, Homo sapiens
ccaaatactttcaaaatataagtattattcgagtcttcacaacaaaagatagaggaccca

SEQ ID NO: 304 - DNA, 60 bp, Homo sapiens
ttccacaaagtttattttttgtttcatttcgaggctacagtggagcctcggcttcccaaa

SEQ ID NO: 305 - DNA, 60 bp, Homo sapiens
taagacagtgattccattcgtaatagtatcgagttgttatttataattcggaactattct

SEQ ID NO: 306 - DNA, 60 bp, Homo sapiens
agttcaatggacaaataaatgaaataaatcgacatactccaaaacattgaaatattgatg

SEQ ID NO: 307 - DNA, 60 bp, Homo sapiens
cattggaggcactcaaaaaaaaaaaaaatcgatgatctcttcaaagacataaccctttgg

SEQ ID NO: 308 - DNA, 60 bp, Homo sapiens
caccttggttataatgtatttttaaatttcgagttcttatgatgctaagagaggctgatt

SEQ ID NO: 309 - DNA, 60 bp, Homo sapiens
ataaatataagttgaatatttattttgttcgaaggatatgaattccagctatggctctgc

SEQ ID NO: 310 - DNA, 60 bp, Homo sapiens
ttttcaaaacaattttaggataagcttgtcgaggttatccctattggttgaaggtgggaa

SEQ ID NO: 311 - DNA, 60 bp, Homo sapiens
ttagtcttaatttataaagcaacttacttcgatgttattgcagcttaaaggtcaggctta

SEQ ID NO: 312 - DNA, 60 bp, Homo sapiens
tatatcagtttgtttatccatttatccatcgaatcaaaatattgtttctacttctttgga

SEQ ID NO: 313 - DNA, 60 bp, Homo sapiens
cctagttagcttattttctctaggattttcgatatgttatttaatcatcaactaattaca

SEQ ID NO: 314 - DNA, 60 bp, Homo sapiens
agaattaaatcttaagtctttatggagatcgactttgaaactaattgtgtttttaccaaa

SEQ ID NO: 315 - DNA, 60 bp, Homo sapiens
ttcttgaaggacatactttaatatttcttcgacactgtctccaacttactttatctgatt

SEQ ID NO: 316 - DNA, 60 bp, Homo sapiens
agctaaagtagatatgcttcttgagaaatcgacacaagggtttgttaaaaaaaaaagaaa

SEQ ID NO: 317 - DNA, 60 bp, Homo sapiens
atagcaagttgcatttagtttttagctatcgattttctttaaacctaaaaaacaagaaaa

SEQ ID NO: 318 - DNA, 60 bp, Homo sapiens
caaaaaaaaaaaaaaaaaaaaaaggtagtcgaaaaataaaatgataaataaaataaaata

SEQ ID NO: 319 - DNA, 60 bp, Homo sapiens
ggttttctttgttttgtttaaaatcatttcgaatctttaattcacattgaatatgtgtac

SEQ ID NO: 320 - DNA, 60 bp, Homo sapiens
gcaaatgcaagagtgcaactaccgtatttcgaaattaattagaagaatatctgcattttt

SEQ ID NO: 321 - DNA, 60 bp, Homo sapiens
agctgctatcatgattactatattattatcgaacagattagatattttaacaccaattct

SEQ ID NO: 322 - DNA, 60 bp, Homo sapiens
tatttatgacagtttaaaaaattccttgtcgagaaatctgctaccttgattagtaagact

SEQ ID NO: 323 - DNA, 60 bp, Homo sapiens
gcagataaaataatactccaataactgatcgaaagtaaaaataaaaagaattacgatgcg

SEQ ID NO: 324 - DNA, 60 bp, Homo sapiens
aaagatgaataaaatttggttcctcccttcgaccctgtgcggacattccgcatactgcca

SEQ ID NO: 325 - DNA, 60 bp, Homo sapiens
gaggttttcaaaaagaattatttaaaagtcgattaaccatttctgccatgtatgaaggat

SEQ ID NO: 326 - DNA, 60 bp, Homo sapiens
tatccacctaccaaattatcacccatcctcgaaaatcttagctttgtaaagaactgttaa

SEQ ID NO: 327 - DNA, 60 bp, Homo sapiens
gtttaaaggacaatatgtcaatagattctcgaaactattttgtttgtaactaataagtga

SEQ ID NO: 328 - DNA, 60 bp, Homo sapiens
gatggatagataaatggaaagatagagatcgagttgtgttaattattaggaaaataacct

SEQ ID NO: 329 - DNA, 60 bp, Homo sapiens
tatccacctaccaaattatcacccatcctcgaaatgttcctggataataaacacacaatt

SEQ ID NO: 330 - DNA, 60 bp, Homo sapiens
aaagtaataatgtaaaactatcttttcctcgatatttgtaggtggttgaagattaatgtt

SEQ ID NO: 331 - DNA, 60 bp, Homo sapiens
actttagataatctgtatatgttttgcatcgaagaatataatggtatatgccatgttaaa

SEQ ID NO: 332 - DNA, 60 bp, Homo sapiens
acctcattcaaaaatcatcttcttgatatcgaacagtaaaaggacttcctctttttttgg

SEQ ID NO: 333 - DNA, 60 bp, Homo sapiens
atttacttatttattcatggaatgtttatcgaggctgatttagacaggactttgtgtgca

SEQ ID NO: 334 - DNA, 60 bp, Homo sapiens
gccagatatttggtgcgcagatatttggtcgataaatgtttattgccctttactgtgtga

SEQ ID NO: 335 - DNA, 60 bp, Homo sapiens
tttgtcatgagtataaaatgagaattcatcgaatctgattcctcttttagactgtaagtt

SEQ ID NO: 336 - DNA, 60 bp, Homo sapiens
caagttctcccaactaataattggaagttcgaaattccaaatatggaaacacatactgaa

SEQ ID NO: 337 - DNA, 60 bp, Homo sapiens
attccactgtaacaagtaaagaaaatattcgaggtacactttaggcggtaccctgttacc

SEQ ID NO: 338 - DNA, 60 bp, Homo sapiens
gcaagaatccaaaatttactgtgctttatcgactatacaataacagtgaatttatggtgt

SEQ ID NO: 339 - DNA, 60 bp, Homo sapiens
cccatggaaaattaaatgatactaaacatcgatttaataaggtcagacaggagctaggta

SEQ ID NO: 340 - DNA, 60 bp, Homo sapiens
atattttatagttgcaggaagctactattcgacactccagagagctctttctatacatag

SEQ ID NO: 341 - DNA, 60 bp, Homo sapiens
attttctcatgatcaaaacattctgctttcgatagttcagtcaccaggtgactgtcgtgg

SEQ ID NO: 342 - DNA, 60 bp, Homo sapiens
caagttctcccaactaataattggaagttcgaatggctttattctaaaatatataaatta

SEQ ID NO: 343 - DNA, 60 bp, Homo sapiens
aatgaaacagttaagaggaatgctattttcgaccttctatagttctctcattttcagtat

SEQ ID NO: 344 - DNA, 60 bp, Homo sapiens
aacagtcaaaaaacaatagatattggtatcgatgttctaggagagctcttaatacagtta

SEQ ID NO: 345 - DNA, 60 bp, Homo sapiens
tatactaagcaaaactcaggtaagaatttcgattttctttaaacctaaaaaacaagaaaa

SEQ ID NO: 346 - DNA, 60 bp, Homo sapiens
acctgtttaactttttaattggttctactcgataaagaatcacaaatctccatcttgcga

SEQ ID NO: 347 - DNA, 60 bp, Homo sapiens
tatttcacttagcataatatcttcaaggtcgactttttgcctactttaccttttattatc

SEQ ID NO: 348 - DNA, 60 bp, Homo sapiens
tttatgaaatatagatgcatacactttgtcgagcatcaaacatgtactagatccttttat

SEQ ID NO: 349 - DNA, 60 bp, Homo sapiens
ttacaagtggaatagaatttattggctatcgaattaaatttataattattttcttttaga

SEQ ID NO: 350 - DNA, 60 bp, Homo sapiens
tgcctttattttcctggaaaattctatgtcgacagaactactgaaattgtaaagaaactc

SEQ ID NO: 351 - DNA, 60 bp, Homo sapiens
aattaacagtgtctattttactgaagcttcgaactataaatccacagttctagttgtctt

SEQ ID NO: 352 - DNA, 24 bp, Homo sapiens
gcttgtattggagactaaacatag

SEQ ID NO: 353 - DNA, 24 bp, Homo sapiens
cctctaaaactcatcaataacaat

SEQ ID NO: 354 - DNA, 26 bp, Homo sapiens
tctcttatctcttcattggtaactgc

SEQ ID NO: 355 - DNA, 24 bp, Homo sapiens
tagtcctcaggtttaggcaagttg

SEQ ID NO: 356 - DNA, 26 bp, Homo sapiens
actgttctgttgattggttggtgata

SEQ ID NO: 357 - DNA, 26 bp, Homo sapiens
agggacacagaagccaaatgagagag

SEQ ID NO: 358 - DNA, 25 bp, Homo sapiens
catcaagttttcagaatagtaaatc

SEQ ID NO: 359 - DNA, 24 bp, Homo sapiens
cacctgggaacattatttggcata

SEQ ID NO: 360 - DNA, 23 bp, Homo sapiens
cctgggcaacagagtgagattct

SEQ ID NO: 361 - DNA, 23 bp, Homo sapiens
cctttcctggtccctcctcatca

SEQ ID NO: 362 - DNA, 26 bp, Homo sapiens
caggaccattgagagaataacagata

SEQ ID NO: 363 - DNA, 23 bp, Homo sapiens
gttcctgagccgcccgataaagc

SEQ ID NO: 364 - DNA, 20 bp, Homo sapiens
caccttggttataggacatc

SEQ ID NO: 365 - DNA, 20 bp, Homo sapiens
gagttcttatgatgctaaga

SEQ ID NO: 366 - DNA, 24 bp, Homo sapiens
tgctcatttctgtatccacaccat

SEQ ID NO: 367 - DNA, 24 bp, Homo sapiens
tatgtggcttgtcatccttatttt

SEQ ID NO: 368 - DNA, 26 bp, Homo sapiens
ggctatgtcccctaaggaaacacaca

SEQ ID NO: 369 - DNA, 26 bp, Homo sapiens
caccaggttgacatcttttcagtggg

SEQ ID NO: 370 - DNA, 26 bp, Homo sapiens
gaaactgccctgagatgagagtcctt

SEQ ID NO: 371 - DNA, 26 bp, Homo sapiens
gctgtggatgaaaggaaggagagaca

SEQ ID NO: 372 - DNA, 26 bp, Homo sapiens
taagcagtcattccctgatgccccta

SEQ ID NO: 373 - DNA, 26 bp, Homo sapiens
cacctgagcaatgcggataaagcaga

SEQ ID NO: 374 - DNA, 26 bp, Homo sapiens
atgcctcaaccattacatcttttcct

SEQ ID NO: 375 - DNA, 26 bp, Homo sapiens
agtagacagatgggtgggattcttcc

SEQ ID NO: 376 - DNA, 26 bp, Homo sapiens
gaggaagaggaatgtccgctaacaca

SEQ ID NO: 377 - DNA, 26 bp, Homo sapiens
gccagcacccaagatttagccaagtt

SEQ ID NO: 378 - DNA, 26 bp, Homo sapiens
ttctattgtgtttcgtggttcctgga

SEQ ID NO: 379 - DNA, 25 bp, Homo sapiens
atacctgagtaggagttagcgtcta

SEQ ID NO: 380 - DNA, 24 bp, Homo sapiens
tcaactatcaatgcgaaagtaaat

SEQ ID NO: 381 - DNA, 25 bp, Homo sapiens
cacatttcccatctcattattatta

SEQ ID NO: 382 - DNA, 24 bp, Homo sapiens
ttggattcatagcagttctaaaat

SEQ ID NO: 383 - DNA, 24 bp, Homo sapiens
tatcaatgtattatctctgcctat

SEQ ID NO: 384 - DNA, 23 bp, Homo sapiens
ggcaacagagcgagactccttct

SEQ ID NO: 385 - DNA, 23 bp, Homo sapiens
aggctgggattacaggcgtaggc

SEQ ID NO: 386 - DNA, 24 bp, Homo sapiens
tatccttccagtctaccttctttt

SEQ ID NO: 387 - DNA, 24 bp, Homo sapiens
gttctgtggtctcctatctacata

SEQ ID NO: 388 - DNA, 24 bp, Homo sapiens
ctgactgactatgattgtttgtag

SEQ ID NO: 389 - DNA, 24 bp, Homo sapiens
atttggcatttcacttttggttta

SEQ ID NO: 390 - DNA, 23 bp, Homo sapiens
tgggctctggagttgggatgcca

SEQ ID NO: 391 - DNA, 23 bp, Homo sapiens
tgttggttgtgtgccagcagcca

SEQ ID NO: 392 - DNA, 23 bp, Homo sapiens
caagtctgctattgaacccctct

SEQ ID NO: 393 - DNA, 23 bp, Homo sapiens
atttccttctgctccccaagtgg

SEQ ID NO: 394 - DNA, 24 bp, Homo sapiens
gctgagagagtcaagggaaaaccc

SEQ ID NO: 395 - DNA, 24 bp, Homo sapiens
cgtggaatctcctcttctcacccc

SEQ ID NO: 396 - DNA, 23 bp, Homo sapiens
gaccaaacgactcacagccaggc

SEQ ID NO: 397 - DNA, 23 bp, Homo sapiens
cttggcagtggagacatacagcg

SEQ ID NO: 398 - DNA, 23 bp, Homo sapiens
cccaagcagggcaaccatcaagg

SEQ ID NO: 399 - DNA, 23 bp, Homo sapiens
acaatcagtgggctggatgtggc

SEQ ID NO: 400 - DNA, 26 bp, Homo sapiens
aaaatgtctgtccccaccatcccagg

SEQ ID NO: 401 - DNA, 25 bp, Homo sapiens
tgacttctcatcaggtccgagtgta

SEQ ID NO: 402 - DNA, 26 bp, Homo sapiens
cagaggagtgggttattttcaaggga

SEQ ID NO: 403 - DNA, 26 bp, Homo sapiens
ttgatttgccctcagtaacctcgctg

SEQ ID NO: 404 - DNA, 26 bp, Homo sapiens
atgtatgtgtggatagacggatggat

SEQ ID NO: 405 - DNA, 26 bp, Homo sapiens
ccctttggacgggaatacctctcact

SEQ ID NO: 406 - DNA, 23 bp, Homo sapiens
cccaccatcccaggcaatcccaa

SEQ ID NO: 407 - DNA, 23 bp, Homo sapiens
ggaaatcactgggtctgttgccc

SEQ ID NO: 408 - DNA, 24 bp, Homo sapiens
aaaatcaggagtttggagaaaata

SEQ ID NO: 409 - DNA, 24 bp, Homo sapiens
tcataaaagtagtcagcaggaaaa

SEQ ID NO: 410 - DNA, 26 bp, Homo sapiens
gagacctcatctgtggtgtttcctgg

SEQ ID NO: 411 - DNA, 26 bp, Homo sapiens
cttgttgaagtgagggattttgtcct

SEQ ID NO: 412 - DNA, 23 bp, Homo sapiens
tccaccagggagagacacaagga

SEQ ID NO: 413 - DNA, 23 bp, Homo sapiens
tcccctggattgtgtgctgggca

SEQ ID NO: 414 - DNA, 24 bp, Homo sapiens
taaccccagcctaagacaaaatag

SEQ ID NO: 415 - DNA, 24 bp, Homo sapiens
ccattttgtttggttttgctttgc

SEQ ID NO: 416 - DNA, 25 bp, Homo sapiens
ggaagggaagatacagagggtttct

SEQ ID NO: 417 - DNA, 23 bp, Homo sapiens
aaacaagcggcggcaggacccct

SEQ ID NO: 418 - DNA, 26 bp, Homo sapiens
taaaatgggcataatcatagcagcca

SEQ ID NO: 419 - DNA, 26 bp, Homo sapiens
ggtcattgattagggaatagtaacac

SEQ ID NO: 420 - DNA, 24 bp, Homo sapiens
gagagggagggtgagagagaacct

SEQ ID NO: 421 - DNA, 23 bp, Homo sapiens
gggcttggtgtttccttggttcc

SEQ ID NO: 422 - DNA, 26 bp, Homo sapiens
ccatccaaaagagtcatacacaggaa

SEQ ID NO: 423 - DNA, 26 bp, Homo sapiens
gtttgcttagtccttactgcttgcct

SEQ ID NO: 424 - DNA, 26 bp, Homo sapiens
gcttctgtctccttcttttcagtctc

SEQ ID NO: 425 - DNA, 26 bp, Homo sapiens
tagattacacatacatcctccattgg

SEQ ID NO: 426 - DNA, 24 bp, Homo sapiens
gcatttactttgtgcttatttgta

SEQ ID NO: 427 - DNA, 24 bp, Homo sapiens
ttactctaaacaagccactgaagt

SEQ ID NO: 428 - DNA, 24 bp, Homo sapiens
cttttggactattgattttcctat

SEQ ID NO: 429 - DNA, 24 bp, Homo sapiens
aaagcacaggcagatgtatgaata

SEQ ID NO: 430 - DNA, 26 bp, Homo sapiens
gaggaggaaaatagaccttcagaggc

SEQ ID NO: 431 - DNA, 26 bp, Homo sapiens
aagaccagacacatttcaacgccacg

SEQ ID NO: 432 - DNA, 26 bp, Homo sapiens
gagggagggtgagagagaacctatct

SEQ ID NO: 433 - DNA, 26 bp, Homo sapiens
ggagtatgattgtccatcttctgcct

SEQ ID NO: 434 - DNA, 26 bp, Homo sapiens
tacttctgcgtgctttattccaggca

SEQ ID NO: 435 - DNA, 26 bp, Homo sapiens
ccagaggatgacttgatttggagtgg

SEQ ID NO: 436 - DNA, 24 bp, Homo sapiens
caacatcactaatcatcagagata

SEQ ID NO: 437 - DNA, 24 bp, Homo sapiens
ttggtgttctcatagcatcttcta

SEQ ID NO: 438 - DNA, 26 bp, Homo sapiens
gctgggaagcagaaggatttggagtt

SEQ ID NO: 439 - DNA, 26 bp, Homo sapiens
tcagtcctctgcttttatctttgaga

SEQ ID NO: 440 - DNA, 26 bp, Homo sapiens
ggaggcaggtttcttttcacatccac

SEQ ID NO: 441 - DNA, 26 bp, Homo sapiens
tgttatttacgaagagtgttgtgagc

SEQ ID NO: 442 - DNA, 24 bp, Homo sapiens
cgtcattctactttctgtctccat

SEQ ID NO: 443 - DNA, 24 bp, Homo sapiens
gtaataagtgagagtcatacaata

SEQ ID NO: 444 - DNA, 24 bp, Homo sapiens
tttctttgtttcatagtgaggtga

SEQ ID NO: 445 - DNA, 24 bp, Homo sapiens
gtaatacagaaatagtaacaccta

SEQ ID NO: 446 - DNA, 24 bp, Homo sapiens
tcagtgttcaaatggatgacaaaa

SEQ ID NO: 447 - DNA, 24 bp, Homo sapiens
agaactacatctgaaaatcaaagt

SEQ ID NO: 448 - DNA, 28 bp, Homo sapiens
gtttgtttagatgactgtctcttatcag

SEQ ID NO: 449 - DNA, 23 bp, Homo sapiens
ctttacaggcagacccagggagc

SEQ ID NO: 450 - DNA, 23 bp, Homo sapiens
gggcacataccccttcctggtct

SEQ ID NO: 451 - DNA, 23 bp, Homo sapiens
gtgcctaaagccacaggagtgag

SEQ ID NO: 452 - DNA, 60 bp, Homo sapiens
gagaatcaattccatttttaaagcttagtcgagtcccttacagaaaatgatgtagtattt

SEQ ID NO: 453 - DNA, 60 bp, Homo sapiens
gtcacctattttttccaacttaatttgatcgattcattacactttatattaagtagtttt

SEQ ID NO: 454 - DNA, 60 bp, Homo sapiens
tttgtaacaatagcaaaagttcagttcttcgagctaatagaaggatggtgaagtaaattt

SEQ ID NO: 455 - DNA, 60 bp, Homo sapiens
taccttccaacaggaagtgcaaactaattcgaaataattcagacatgattgtgttttact

SEQ ID NO: 456 - DNA, 60 bp, Homo sapiens
tttgtaacaatagcaaaagttcagttcttcgataaacatttattcaaaggcatgaatgac

SEQ ID NO: 457 - DNA, 60 bp, Homo sapiens
gagaatcaattccatttttaaagcttagtcgaatgctaatcctcttctattaatgaattc

SEQ ID NO: 458 - DNA, 60 bp, Homo sapiens
gtcacagagatcatggtaaagagaaaattcgattacataaatatttctctaaggttgctg

SEQ ID NO: 459 - DNA, 60 bp, Homo sapiens
agaaatgaacaacacattgtaatatacatcgaaaagaactaataatgctaaggattctga

SEQ ID NO: 460 - DNA, 60 bp, Homo sapiens
agaagactcaccaaaattttatcctgtttcgaaccttataatggtgataaatcattaatg

SEQ ID NO: 461 - DNA, 60 bp, Homo sapiens
aaatcaagacggaggaactatttcagattcgaggtagtggcagctaatagctcttaaggg

SEQ ID NO: 462 - DNA, 60 bp, Homo sapiens
ttgaagtctgtttgtgaaatactttgagtcgaaagacaaacggcaaaatgagaaaacaat

SEQ ID NO: 463 - DNA, 60 bp, Homo sapiens
cgcgtgatgtgtttgaaggtgtgtttgttcgaaaaagacttgcatttatgcagagtgagg

SEQ ID NO: 464 - DNA, 60 bp, Homo sapiens
gagaatcaattccatttttaaagcttagtcgattcttgcaaatgaattttgttaaatgtt

SEQ ID NO: 465 - DNA, 60 bp, Homo sapiens
aaattattttgctttctatttctcctcttcgatctttcatatttcatcctggtttcacaa

SEQ ID NO: 466 - DNA, 60 bp, Homo sapiens
agttttctcagttattgaaatgaatctctcgaagtatgtactttggtatgtccaaaagtc

SEQ ID NO: 467 - DNA, 60 bp, Homo sapiens
aatgaagtcccaggctttcgtctcacgttcgaataacaaccagtttaaaaacatactaaa

SEQ ID NO: 468 - DNA, 60 bp, Homo sapiens
tttctaaatttccaaggttctttccagttcgaccaatgaaactgattaagaccatacaag

SEQ ID NO: 469 - DNA, 60 bp, Homo sapiens
gcatgaacaactgtttttcactttgatttcgaatttcttgacttttgactggttgtttat

SEQ ID NO: 470 - DNA, 60 bp, Homo sapiens
gtcacctattttttccaacttaatttgatcgatttctgcatttccttgaactgaatctta

SEQ ID NO: 471 - DNA, 60 bp, Homo sapiens
agaaatgaacaacacattgtaatatacatcgaaactgtcactacattttacctaatttag

SEQ ID NO: 472 - DNA, 60 bp, Homo sapiens
ctcaagaaagaaaacctaagctttaaattcgactctaatctataaaactaagatacttaa

SEQ ID NO: 473 - DNA, 60 bp, Homo sapiens
caactgaggaactggaacttcattagagtcgaagtgctctgtggtgatgcagcaggttga

SEQ ID NO: 474 - DNA, 60 bp, Homo sapiens
agaagctgtatgatttgtagtaattcattcgaatagtcagtagctgcctttgcaaagatg

SEQ ID NO: 475 - DNA, 60 bp, Homo sapiens
ccaaaacagtcccaaaacaggagagctctcgatgggactattggaagatttctcagaaga

SEQ ID NO: 476 - DNA, 60 bp, Homo sapiens
tggtgcataatgaagactaaagcgttcctcgatttaaaaatagtttattttaataatgtt

SEQ ID NO: 477 - DNA, 60 bp, Homo sapiens
tttctaaatttccaaggttctttccagttcgaggcaattctaaagaacaataagagatta

SEQ ID NO: 478 - DNA, 60 bp, Homo sapiens
ggtgagtggcagatagctgggaaaacattcgagccaagctgagtgacagctttgtcacgg

SEQ ID NO: 479 - DNA, 60 bp, Homo sapiens
gcaagttgtatgggaggtagtttaaatatcgaagtatctcaaataacatttttgagaatg

SEQ ID NO: 480 - DNA, 60 bp, Homo sapiens
gagaatcaattccatttttaaagcttagtcgattaggatgatggcaatggaaataacaag

SEQ ID NO: 481 - DNA, 60 bp, Homo sapiens
agctcaggcaatccatgcttggagaaagtcgatagatggcaaatttcttgatacggggag

SEQ ID NO: 482 - DNA, 60 bp, Homo sapiens
ttgtgaggagctaagggagactcactgatcgaaaaagatttatgaacagaaaaaggaaag

SEQ ID NO: 483 - DNA, 60 bp, Homo sapiens
caacaagaacgactttcttttttcttcttcgactatgataatctttctcatttatagacc

SEQ ID NO: 484 - DNA, 60 bp, Homo sapiens
actagcttgcaaggattgttgcaaagtttcgatgaaagcacaaataaataattaaaggca

SEQ ID NO: 485 - DNA, 60 bp, Homo sapiens
agcgaatttaaggcagtaataagtgcagtcgaggcagtgggagttgaatcctctcccctc

SEQ ID NO: 486 - DNA, 60 bp, Homo sapiens
attaggagtgggggccttgagaggtaattcgatcttttcaaagaaccacattttagtttt

SEQ ID NO: 487 - DNA, 60 bp, Homo sapiens
gagctgcaaggccttttaaagctcagcctcgatttgtattccacagagcttatcattctc

SEQ ID NO: 488 - DNA, 60 bp, Homo sapiens
aatactactttgcatatataataaagtttcgagaatggactaataaacacatgtttctga

SEQ ID NO: 489 - DNA, 60 bp, Homo sapiens
tcagtctagaacataacagaaaatgtcttcgactctttttacaaaaggccacttgacatc

SEQ ID NO: 490 - DNA, 60 bp, Homo sapiens
aatgtacacaaccagtcataaccaagtatcgaaaagatttttacagagttttcccccttc

SEQ ID NO: 491 - DNA, 60 bp, Homo sapiens
ggcagatggatcacttgaggtcaagagttcgagatgcagccagatctcggcgaagtaaag

SEQ ID NO: 492 - DNA, 60 bp, Homo sapiens
cttataatgggtagaagcagactatcattcgataagaaaaagagagatatccaaataaaa

SEQ ID NO: 493 - DNA, 60 bp, Homo sapiens
ttggttagagataaaaaccctgtaaatgtcgaccagtgaaataactaaaatactgtgaca

SEQ ID NO: 494 - DNA, 60 bp, Homo sapiens
aggctgatatttgttctttagctgtgtgtcgaatggtagaagggagataatatgggtgca

SEQ ID NO: 495 - DNA, 60 bp, Homo sapiens
gcttacttggtcattagatccacagagttcgactctttaaccacacggcttgcctagttc

SEQ ID NO: 496 - DNA, 60 bp, Homo sapiens
gaggatgaaaattaaatgacttttaatgtcgagaattaaactcagtttaagagactgcta

SEQ ID NO: 497 - DNA, 60 bp, Homo sapiens
ggcaggtggattgcttgagctcaggagttcgaaaaagaaaagaaaagaacataacagaac

SEQ ID NO: 498 - DNA, 60 bp, Homo sapiens
gtagtccaggggaagagaaaaatgtgaatcgataattgtgaaagtcacttccgctctctg

SEQ ID NO: 499 - DNA, 60 bp, Homo sapiens
gtacagttccaacgagcggagtggtgggtcgaagcaatatttaaaagcttagaggatgtt

SEQ ID NO: 500 - DNA, 60 bp, Homo sapiens
gcaagttgtatgggaggtagtttaaatatcgaaatcttctgatctattattgatgacatg

SEQ ID NO: 501 - DNA, 60 bp, Homo sapiens
ctgcagcccgtagttgtggatgatggagtcgatcgtgcagacaggagacaaacagcaaga

SEQ ID NO: 502 - DNA, 26 bp, Homo sapiens
gcccctgtcttctctcaacttcatag

SEQ ID NO: 503 - DNA, 26 bp, Homo sapiens
ccgaatggatggttgtgtctgtgttg

SEQ ID NO: 504 - DNA, 26 bp, Homo sapiens
tcagcctcagaatgccagagtgactg

SEQ ID NO: 505 - DNA, 26 bp, Homo sapiens
cgaagactctgacaaacacagtggta

SEQ ID NO: 506 - DNA, 26 bp, Homo sapiens
gctgctatgaggcattgtagaggaga

SEQ ID NO: 507 - DNA, 26 bp, Homo sapiens
gaacagcagacttgtggaccttatca

SEQ ID NO: 508 - DNA, 26 bp, Homo sapiens
aacaggcagagaggtggcttcagatg

SEQ ID NO: 509 - DNA, 26 bp, Homo sapiens
gagaagcaacaccacaaaagcaggaa

SEQ ID NO: 510 - DNA, 26 bp, Homo sapiens
gctgctatgaggcattgtagaggaga

SEQ ID NO: 511 - DNA, 26 bp, Homo sapiens
gctctctgtccattcctcagtttctc

SEQ ID NO: 512 - DNA, 26 bp, Homo sapiens
ccctgtcttctctcaacttcatagtc

SEQ ID NO: 513 - DNA, 26 bp, Homo sapiens
tagcaggaactatgtgccactgtgcc

SEQ ID NO: 514 - DNA, 23 bp, Homo sapiens
cccactgcttggctttggaggaa

SEQ ID NO: 515 - DNA, 23 bp, Homo sapiens
gcagagactttccaccctgtgcc

SEQ ID NO: 516 - DNA, 23 bp, Homo sapiens
gcccctttctgggaacaacaacg

SEQ ID NO: 517 - DNA, 24 bp, Homo sapiens
ggagcaagtagggaagcagtggtc

SEQ ID NO: 518 - DNA, 26 bp, Homo sapiens
accctgtgcccttgagcatactgaat

SEQ ID NO: 519 - DNA, 26 bp, Homo sapiens
tgccttgtttcccctttgcctttagc

SEQ ID NO: 520 - DNA, 23 bp, Homo sapiens
acctcggagaagcctggcagaca

SEQ ID NO: 521 - DNA, 23 bp, Homo sapiens
gtgcttagtgcctggctcatagc

SEQ ID NO: 522 - DNA, 26 bp, Homo sapiens
agaaagtggcagactcagggtggaag

SEQ ID NO: 523 - DNA, 26 bp, Homo sapiens
ccctttgcgttaggagtttcagattg

SEQ ID NO: 524 - DNA, 23 bp, Homo sapiens
gcgaatgtccctacaagtgtggg

SEQ ID NO: 525 - DNA, 23 bp, Homo sapiens
actggaagcaaaggtgcctgggc

SEQ ID NO: 526 - DNA, 26 bp, Homo sapiens
gcccctgtcttctctcaacttcatag

SEQ ID NO: 527 - DNA, 26 bp, Homo sapiens
ctgatactgtgtagggagtctgatga

SEQ ID NO: 528 - DNA, 23 bp, Homo sapiens
tacggctcctccatctacccagc

SEQ ID NO: 529 - DNA, 25 bp, Homo sapiens
ctcctaagataagtgactccttgtg

SEQ ID NO: 530 - DNA, 26 bp, Homo sapiens
cggtaaatctaaaggacaatgctggt

SEQ ID NO: 531 - DNA, 26 bp, Homo sapiens
gccacagactgaagaggattggttct

SEQ ID NO: 532 - DNA, 26 bp, Homo sapiens
ccgttgctcaagaaccatccagagtc

SEQ ID NO: 533 - DNA, 26 bp, Homo sapiens
gcaaacttcaggctgtattgttctcg

SEQ ID NO: 534 - DNA, 26 bp, Homo sapiens
ggtagattccaactctgattccattt

SEQ ID NO: 535 - DNA, 26 bp, Homo sapiens
ctttccttctctcttccttattgagc

SEQ ID NO: 536 - DNA, 23 bp, Homo sapiens
gctcattggcagaagcaggtttt

SEQ ID NO: 537 - DNA, 23 bp, Homo sapiens
tggcaaagcaacaccctaaggat

SEQ ID NO: 538 - DNA, 26 bp, Homo sapiens
tcagcctcagaatgccagagtgactg

SEQ ID NO: 539 - DNA, 26 bp, Homo sapiens
tgagaaaccacagccacagcagaaat

SEQ ID NO: 540 - DNA, 26 bp, Homo sapiens
ctttaccctcagtttccttatcggga

SEQ ID NO: 541 - DNA, 26 bp, Homo sapiens
gcagagagggagatgttgatggaggt

SEQ ID NO: 542 - DNA, 25 bp, Homo sapiens
taacaactgctaactggagacacca

SEQ ID NO: 543 - DNA, 25 bp, Homo sapiens
acttgctgtctcgtttccttgcccc

SEQ ID NO: 544 - DNA, 26 bp, Homo sapiens
gcaacactactctctctccttccttg

SEQ ID NO: 545 - DNA, 26 bp, Homo sapiens
gcagtattccctttacagttattggt

SEQ ID NO: 546 - DNA, 23 bp, Homo sapiens
gaggcaggcttttcgttccagca

SEQ ID NO: 547 - DNA, 24 bp, Homo sapiens
gagacatcttccccacttgctcgc

SEQ ID NO: 548 - DNA, 26 bp, Homo sapiens
tacaaagggtgaaggagaggaggtcc

SEQ ID NO: 549 - DNA, 26 bp, Homo sapiens
ccctattttctcctggtcagcaagtt

SEQ ID NO: 550 - DNA, 26 bp, Homo sapiens
caccttccattgatactctcactttg

SEQ ID NO: 551 - DNA, 26 bp, Homo sapiens
gcacaagaccaaaggatttatcgcac

SEQ ID NO: 552 - DNA, 26 bp, Homo sapiens
ccacgcctcagccaatcactccaaaa

SEQ ID NO: 553 - DNA, 26 bp, Homo sapiens
gaaagccattgtcccaacaccaagta

SEQ ID NO: 554 - DNA, 26 bp, Homo sapiens
cagtagttctaaaagagaggatggta

SEQ ID NO: 555 - DNA, 26 bp, Homo sapiens
gccagtaaaccaaggcttattaggat

SEQ ID NO: 556 - DNA, 26 bp, Homo sapiens
ggtttggtcgtgaactgtcagaaagg

SEQ ID NO: 557 - DNA, 26 bp, Homo sapiens
ctttgtatttctttccttcagtctgg

SEQ ID NO: 558 - DNA, 23 bp, Homo sapiens
gggttcgcatcacatcccacaga

SEQ ID NO: 559 - DNA, 23 bp, Homo sapiens
cccactcacagtttgctgacccc

SEQ ID NO: 560 - DNA, 26 bp, Homo sapiens
ttatcccctgtctgacttgttccccg

SEQ ID NO: 561 - DNA, 26 bp, Homo sapiens
gcgttacatctgttgtgccagcaaaa

SEQ ID NO: 562 - DNA, 26 bp, Homo sapiens
ggagtgggagtcagagaaagggtctt

SEQ ID NO: 563 - DNA, 26 bp, Homo sapiens
cctcacttgtgctttctgttcatcac

SEQ ID NO: 564 - DNA, 26 bp, Homo sapiens
ctgaagcagactctcactggaataca

SEQ ID NO: 565 - DNA, 26 bp, Homo sapiens
acatagcagtgcccaggagaacaagc

SEQ ID NO: 566 - DNA, 26 bp, Homo sapiens
gctcatccacttccttgcttgcttct

SEQ ID NO: 567 - DNA, 26 bp, Homo sapiens
gatggcaggagtttatggaaagggct

SEQ ID NO: 568 - DNA, 26 bp, Homo sapiens
aacatcagtcaggaaagacaaggttc

SEQ ID NO: 569 - DNA, 26 bp, Homo sapiens
tggtgtgtcctgggttttgtggagca

SEQ ID NO: 570 - DNA, 24 bp, Homo sapiens
cttgttatggaccaaatgtctggc

SEQ ID NO: 571 - DNA, 25 bp, Homo sapiens
cagcagaaggaaagaaataataaaa

SEQ ID NO: 572 - DNA, 23 bp, Homo sapiens
gtcctccgtcttccaacaggcta

SEQ ID NO: 573 - DNA, 23 bp, Homo sapiens
aggcaccatcctaagccctgggt

SEQ ID NO: 574 - DNA, 24 bp, Homo sapiens
gtgttatttgtatctcctcctggc

SEQ ID NO: 575 - DNA, 24 bp, Homo sapiens
attatgtgcttacccaaaagaaat

SEQ ID NO: 576 - DNA, 25 bp, Homo sapiens
tcagacatctcaaaagcagcagttc

SEQ ID NO: 577 - DNA, 25 bp, Homo sapiens
cttttgaactgccaagtgaggatgt

SEQ ID NO: 578 - DNA, 26 bp, Homo sapiens
ggagatgaggagcgaaagaaagggtt

SEQ ID NO: 579 - DNA, 26 bp, Homo sapiens
tgtggtaggacagatagggaagaagg

SEQ ID NO: 580 - DNA, 23 bp, Homo sapiens
gcctcagggaacgcttcttaggg

SEQ ID NO: 581 - DNA, 24 bp, Homo sapiens
ggaagtggcaactgatgagcaagc

SEQ ID NO: 582 - DNA, 26 bp, Homo sapiens
agaataccctatgatggagagttccg

SEQ ID NO: 583 - DNA, 26 bp, Homo sapiens
ccacctcccctactttcccataacat

SEQ ID NO: 584 - DNA, 23 bp, Homo sapiens
cagatgagggcagtcttgggaga

SEQ ID NO: 585 - DNA, 23 bp, Homo sapiens
ctctcagtccaactcccaccgac

SEQ ID NO: 586 - DNA, 23 bp, Homo sapiens
gagataaaggtgtgagccactgt

SEQ ID NO: 587 - DNA, 23 bp, Homo sapiens
catcccagaccatcagccccgct

SEQ ID NO: 588 - DNA, 23 bp, Homo sapiens
ggagtggaggacttgatacaccg

SEQ ID NO: 589 - DNA, 23 bp, Homo sapiens
tgggagtggtttgggaggtgtgc

SEQ ID NO: 590 - DNA, 26 bp, Homo sapiens
tctattctgctcctacacagtgaaat

SEQ ID NO: 591 - DNA, 23 bp, Homo sapiens
gcaaacttgtatccagtgaaggc

SEQ ID NO: 592 - DNA, 23 bp, Homo sapiens
gtaatcccagcactttggtaggc

SEQ ID NO: 593 - DNA, 23 bp, Homo sapiens
gaggcttcctgcttgctgggagc

SEQ ID NO: 594 - DNA, 23 bp, Homo sapiens
gcctgggcaacagagcgagactc

SEQ ID NO: 595 - DNA, 26 bp, Homo sapiens
attacaatacccttgctttgggacaa

SEQ ID NO: 596 - DNA, 26 bp, Homo sapiens
gtattccaaggcagtcaatcctggga

SEQ ID NO: 597 - DNA, 26 bp, Homo sapiens
attagtatgtctgctgaaggcactta

SEQ ID NO: 598 - DNA, 26 bp, Homo sapiens
tgaactgtcagaaaggaggcgaaagc

SEQ ID NO: 599 - DNA, 26 bp, Homo sapiens
ggaatgacaccttcaaatgacaaatg

SEQ ID NO: 600 - DNA, 23 bp, Homo sapiens
caggaactcatcgcccgtgcggc

SEQ ID NO: 601 - DNA, 23 bp, Homo sapiens
tgagaccattccaaagcagaggg

SEQ ID NO: 602 - DNA, 60 bp, Homo sapiens
gggtctccccatattgcctaggctggtctcgagggaactccctccccgcacccccagcat

SEQ ID NO: 603 - DNA, 60 bp, Homo sapiens
cagtccagtctgagtgactctagcgatgtcgactgcccgcccagatcccgcctccagagg

SEQ ID NO: 604 - DNA, 60 bp, Homo sapiens; identical to SEQ ID NO: 195

SEQ ID NO: 605 - DNA, 60 bp, Homo sapiens; identical to SEQ ID NO: 168

SEQ ID NO: 606 - DNA, 60 bp, Homo sapiens
cgcggccgtcagagggcgcggcctacactcgatcttatcctttgaccttgcccactcttt

SEQ ID NO: 607 - DNA, 60 bp, Homo sapiens
aatgcgcagtcttctttccttccttttgtcgaactcctaaccttgtgatctacccgcctc

SEQ ID NO: 608 - DNA, 60 bp, Homo sapiens
ctggtcctgccctcactctggtgcgtagtcgagccagcatgagctctctgttcctgcaga

SEQ ID NO: 609 - DNA, 60 bp, Homo sapiens
atacacaggatagatttcaagatcattctcgatgtacttctctgcctcagcctacgtgtc

SEQ ID NO: 610 - DNA, 60 bp, Homo sapiens
ggatttctccgtgttggccaggctggtctcgagacgaccctggccaacaaggagaaaccc

SEQ ID NO: 611 - DNA, 60 bp, Homo sapiens
gggtttcgccatgttggccaagctggtctcgagaccagccttgccaacatggtgaaaccc

SEQ ID NO: 612 - DNA, 60 bp, Homo sapiens
gggtttcatcatgttggccaggctggtctcgagaccagcctgaccaacaggtgaaaccca

SEQ ID NO: 613 - DNA, 60 bp, Homo sapiens
tctcttccggttctgtcttttcgctggctcgaacaacaaattgttaagtgaatggaatgc

SEQ ID NO: 614 - DNA, 60 bp, Homo sapiens; identical to SEQ ID NO: 190

SEQ ID NO: 615 - DNA, 60 bp, Homo sapiens
actgtaggcagacagggaacttggcatatcgaggtggtcccacaaaaatattttatgtag

SEQ ID NO: 616 - DNA, 60 bp, Homo sapiens
tggagaaaactggtggacactactttaatcgaactattgggctcaagcaatccacccacc

SEQ ID NO: 617 - DNA, 60 bp, Homo sapiens
ggacataactgtattctctctctctctctcgaagctttttcctggggcagattctgcctg

SEQ ID NO: 618 - DNA, 60 bp, Homo sapiens
catgatgcataaactctaaacaggtctttcgagaaatgccacgctcactagaaaatatag

SEQ ID NO: 619 - DNA, 60 bp, Homo sapiens
gggtttcaccatgttggccaggctggtctcgagaccagactggccaacatagtgaaaccc

SEQ ID NO: 620 - DNA, 60 bp, Homo sapiens
cagtcacagcaaatgccacagggtgtgttcgaggaaaagtggcaaatgtctctctactcc

SEQ ID NO: 621 - DNA, 60 bp, Homo sapiens
tgatctggaaaaaaaatcaatttagatctcgaagctgtcatctgtgggcgcgggttgagc

SEQ ID NO: 622 - DNA, 60 bp, Homo sapiens
ggatttatccgtgttggtcaggctggtctcgagacaagcctgactaacatggtaaaaccc

SEQ ID NO: 623 - DNA, 60 bp, Homo sapiens
ggacataactgtattctctctctctctctcgagcaggatgtggaacagacatcatgagcc

SEQ ID NO: 624 - DNA, 60 bp, Homo sapiens
tctcttccggttctgtcttttcgctggctcgagataagtggacccagctgtttattccca

SEQ ID NO: 625 - DNA, 60 bp, Homo sapiens
cagactttggaactaagctgacttatattcgagatggagttttgctctttttgcccaggc

SEQ ID NO: 626 - DNA, 60 bp, Homo sapiens
ggcaggtggaccacctgaggtcaggagttcgaactcctggactcaagtgatcttcccacc

SEQ ID NO: 627 - DNA, 60 bp, Homo sapiens
gtaacagcagcaatggcagtggtggtgatcgaaatgcccatgaagtgagccaaaactttg

SEQ ID NO: 628 - DNA, 60 bp, Homo sapiens
acaacaacaaaaaagaaccaaatagtgatcgacttgggagtgcctgcgttcccgcggctc

SEQ ID NO: 629 - DNA, 60 bp, Homo sapiens
gggtttctccatgttggtcaggctggtctcgagaccagtctgaccaacatggtgaaaccc

SEQ ID NO: 630 - DNA, 60 bp, Homo sapiens
tttgtagtaagtttaattaacttaattttcgatacttcccctgggtgactctgacagatg

SEQ ID NO: 631 - DNA, 60 bp, Homo sapiens
gaaacacaactacaaggtgatgagattatcgatggggagagtccaaggattttagctgtg

SEQ ID NO: 632 - DNA, 60 bp, Homo sapiens
tagggaagtgggtggtgctcaggaggtgtcgagccgaggagcccctctgccctccctgtc

SEQ ID NO: 633 - DNA, 60 bp, Homo sapiens
ggcaggtggatcatttgaggtcaggagttcgaattcctgagctcaaatgatcctcctgcc

SEQ ID NO: 634 - DNA, 60 bp, Homo sapiens
cacaccaccgttgggatgaaagatggcctcgaagttgatgcaatcggtttaaacatggct

SEQ ID NO: 635 - DNA, 60 bp, Homo sapiens
tgtgccttcacccaaactatctgatccctcgacccttaaaaaaaaaaattagccaggcat

SEQ ID NO: 636 - DNA, 60 bp, Homo sapiens
cctaccaggagtccccgccacgctcccctcgacgatgacaccataaccctggcaacaaaa

SEQ ID NO: 637 - DNA, 60 bp, Homo sapiens
tcccttgctctctctctgtctctcagattcgagctcttcttcggcgtctgcagcctcttc

SEQ ID NO: 638 - DNA, 60 bp, Homo sapiens
aagccatggtggcagcgcggcggttggttcgagtatcagataaatgaaaatgctgagact

SEQ ID NO: 639 - DNA, 60 bp, Homo sapiens
tggactcggggtgcccggttccacccactcgaagtgatggaactcataggaccagctcaa

SEQ ID NO: 640 - DNA, 60 bp, Homo sapiens
ggacataactgtattctctctctctctctcgaattcctgacctcggccttccaaagtgct

SEQ ID NO: 641 - DNA, 60 bp, Homo sapiens
ggatttcatcatgttggccaggctggtctcgagaccagtctggccaacatggtaaaaccc

SEQ ID NO: 642 - DNA, 60 bp, Homo sapiens
tagcaaagcagagttgggggtgcgattgtcgaggcctaacaattttccagggtccaaagg

SEQ ID NO: 643 - DNA, 60 bp, Homo sapiens
aggtttcaccatgttggccaggctggtctcgagaccaacctggccaacatggtgcaaccc

SEQ ID NO: 644 - DNA, 60 bp, Homo sapiens
caacgctcacgccaatggtctgagttaatcgaatgcatcctggccatggttctgcagctc

SEQ ID NO: 645 - DNA, 60 bp, Homo sapiens
tggtgagcagaaggctccagctgtacgctcgattcctgttttcccttctgcctccctcgt

SEQ ID NO: 646 - DNA, 60 bp, Homo sapiens
ttgcgctccaggccggctgccatggagctcgaccgccagggcccgcgtcctctccccggc

SEQ ID NO: 647 - DNA, 60 bp, Homo sapiens
aacaaggacttaagtgtgtccacttatgtcgagcaatccttctgcctcagcctcctgagt

SEQ ID NO: 648 - DNA, 60 bp, Homo sapiens
ttcaaacagtttagatacatcaagtccctcgaggctgcagaaggctgggtgggagtggag

SEQ ID NO: 649 - DNA, 60 bp, Homo sapiens
ctctcaataaagttttctaaaattagactcgatggttccctattaccctctgcccgcagc

SEQ ID NO: 650 - DNA, 60 bp, Homo sapiens
gatgtcttggtttttccatgagaaacattcgaggtgctcggcgggcagcgcggggctcaa

SEQ ID NO: 651 - DNA, 60 bp, Homo sapiens
gggtttcaccatgttggtcaggctggtctcgagaccagcctgagcaacatgaagaaaccc

SEQ ID NO: 652 - DNA, 60 bp, Homo sapiens
tagggcttttacagttacagaggagaagtcgacggagtcggttcagtttggggcagatca

SEQ ID NO: 653 - DNA, 23 bp, Homo sapiens
caggctgtctcattctgttgccc

SEQ ID NO: 654 - DNA, 23 bp, Homo sapiens
gccacatacagtcagggtccagc

SEQ ID NO: 655 - DNA, 21 bp, Homo sapiens
ttaggagtgtcccaaaccccc

SEQ ID NO: 656 - DNA, 21 bp, Homo sapiens
tttacgtaggcgcagcacagc

SEQ ID NO: 657 - DNA, 26 bp, Homo sapiens
aagaaacaaagcgaggcaaggtgacg

SEQ ID NO: 658 - DNA, 26 bp, Homo sapiens
aggcagacttctttggagggtgtcaa

SEQ ID NO: 659 - DNA, 22 bp, Homo sapiens
atcttggctcactgcaacctcc

SEQ ID NO: 660 - DNA, 22 bp, Homo sapiens
gctggagtgcaatggcgcaatc

SEQ ID NO: 661 - DNA, 23 bp, Homo sapiens
tcgctcattcctcccgaccaagg

SEQ ID NO: 662 - DNA, 23 bp, Homo sapiens
ctgaagacagagtgagccagcca

SEQ ID NO: 663 - DNA, 26 bp, Homo sapiens
tcatcatacttctccctttccaatct

SEQ ID NO: 664 - DNA, 26 bp, Homo sapiens
tctgccagccctaatccctctgaata

SEQ ID NO: 665 - DNA, 23 bp, Homo sapiens
cctcctcctctttgcccctccta

SEQ ID NO: 666 - DNA, 23 bp, Homo sapiens
ggtgaagatggtgtctgagcctc

SEQ ID NO: 667 - DNA, 23 bp, Homo sapiens
gacacctggagggcagcactgat

SEQ ID NO: 668 - DNA, 23 bp, Homo sapiens
ggtagttggctatctggacctgg

SEQ ID NO: 669 - DNA, 21 bp, Homo sapiens
tggcacagtctcagctcactg

SEQ ID NO: 670 - DNA, 21 bp, Homo sapiens
tctcgttctgttgcccaggct

SEQ ID NO: 671 - DNA, 23 bp, Homo sapiens
ctgggtctccagcctcactcagg

SEQ ID NO: 672 - DNA, 23 bp, Homo sapiens
ctggagtgcagtggcaagttctc

SEQ ID NO: 673 - DNA, 26 bp, Homo sapiens
gtcttgctttgttgctcaggttggag

SEQ ID NO: 674 - DNA, 26 bp, Homo sapiens
gcgcctggtctttttttttttttctg

SEQ ID NO: 675 - DNA, 26 bp, Homo sapiens
cgggtacggggccggtctccccgccc

SEQ ID NO: 676 - DNA, 26 bp, Homo sapiens
aaacgaagacatgagtcactagtaag

SEQ ID NO: 677 - DNA, 24 bp, Homo sapiens
ctcactgcaacttctgcctcccag

SEQ ID NO: 678 - DNA, 24 bp, Homo sapiens
atcctttttttttttttctgagac

SEQ ID NO: 679 - DNA, 23 bp, Homo sapiens
ttgccacccaagagtgatgcggg

SEQ ID NO: 680 - DNA, 23 bp, Homo sapiens
ggctgaccaccaccaaatgtgac

SEQ ID NO: 681 - DNA, 26 bp, Homo sapiens
ctcaaaggatttccccacaagatact

SEQ ID NO: 682 - DNA, 26 bp, Homo sapiens
gcttttctttgtttggtagggttatg

SEQ ID NO: 683 - DNA, 26 bp, Homo sapiens
ggataaagcacaggatgccccagtta

SEQ ID NO: 684 - DNA, 26 bp, Homo sapiens
gaaggtgacagaagacccagaaaagg

SEQ ID NO: 685 - DNA, 26 bp, Homo sapiens
ctgagcttgcttttaattattataaa

SEQ ID NO: 686 - DNA, 26 bp, Homo sapiens
cctgagtacaaacccaatgccagttg

SEQ ID NO: 687 - DNA, 23 bp, Homo sapiens
atgatctaggctcactgcaaact

SEQ ID NO: 688 - DNA, 23 bp, Homo sapiens
tgcctcctgggttcaagcgattc

SEQ ID NO: 689 - DNA, 26 bp, Homo sapiens
tggacacatccgtatgagagccacag

SEQ ID NO: 690 - DNA, 26 bp, Homo sapiens
aacagccaggcaccattgaacatttc

SEQ ID NO: 691 - DNA, 23 bp, Homo sapiens
taaatgaaattaaaaaaaacaaa

SEQ ID NO: 692 - DNA, 23 bp, Homo sapiens
ctcggctccgcaaagacattctg

SEQ ID NO: 693 - DNA, 21 bp, Homo sapiens
gagtttcactcttgttgccca

SEQ ID NO: 694 - DNA, 21 bp, Homo sapiens
cgctctgtcatgcaggctgaa

SEQ ID NO: 695 - DNA, 26 bp, Homo sapiens
ggataaagcacaggatgccccagtta

SEQ ID NO: 696 - DNA, 26 bp, Homo sapiens
tacactgtctcacatttctccctggc

SEQ ID NO: 697 - DNA, 21 bp, Homo sapiens
cgtcccctcgtcccctcgggt

SEQ ID NO: 698 - DNA, 21 bp, Homo sapiens
agagactcttgttacaaccca

SEQ ID NO: 699 - DNA, 21 bp, Homo sapiens
tcattaatattcattaatgac

SEQ ID NO: 700 - DNA, 21 bp, Homo sapiens
actggagcatgcctgtaatcc

SEQ ID NO: 701 - DNA, 25 bp, Homo sapiens
agccagacagatcctgtgaaaagat

SEQ ID NO: 702 - DNA, 25 bp, Homo sapiens
atgcctggaatcccaacattttggg

SEQ ID NO: 703 - DNA, 23 bp, Homo sapiens
gactggagtgctctgctcatctc

SEQ ID NO: 704 - DNA, 23 bp, Homo sapiens
cacccaaagtccccacctgctca

SEQ ID NO: 705 - DNA, 24 bp, Homo sapiens
cctaccattgcccacttgagaggg

SEQ ID NO: 706 - DNA, 23 bp, Homo sapiens
tccacagggcaggttttgatggg

SEQ ID NO: 707 - DNA, 23 bp, Homo sapiens
cctgcgttcaagcgattcttctg

SEQ ID NO: 708 - DNA, 23 bp, Homo sapiens
tgggttcaagcgattctcctgcc

SEQ ID NO: 709 - DNA, 26 bp, Homo sapiens
gctattgagtttgtagtctgaatgac

SEQ ID NO: 710 - DNA, 26 bp, Homo sapiens
gcagtgggttctcaaatgttagcatc

SEQ ID NO: 711 - DNA, 26 bp, Homo sapiens
cgtccctttctggtaatcggtggaag

SEQ ID NO: 712 - DNA, 26 bp, Homo sapiens
cttcatttccatttcctgacacacag

SEQ ID NO: 713 - DNA, 23 bp, Homo sapiens
ggtcaccaacaaggcttctgtgg

SEQ ID NO: 714 - DNA, 23 bp, Homo sapiens
agtccagtcaggttccagcccag

SEQ ID NO: 715 - DNA, 25 bp, Homo sapiens
gggtatggagtgcatacctatggtc

SEQ ID NO: 716 - DNA, 25 bp, Homo sapiens
agacaagagctgattttcccattat

SEQ ID NO: 717 - DNA, 23 bp, Homo sapiens
cttgaaggcggattcctccaggt

SEQ ID NO: 718 - DNA, 23 bp, Homo sapiens
gtgggttgcgtcagtcccgtgta

SEQ ID NO: 719 - DNA, 26 bp, Homo sapiens
tcagtggagtgtgtggaaaatacggc

SEQ ID NO: 720 - DNA, 26 bp, Homo sapiens
cccttcttaccaaacctgtcacccta

SEQ ID NO: 721 - DNA, 23 bp, Homo sapiens
gctctcccaaaaccgtgttcccg

SEQ ID NO: 722 - DNA, 23 bp, Homo sapiens
ccctgtttctgtcctcctccact

SEQ ID NO: 723 - DNA, 26 bp, Homo sapiens
gctctctctctctccctctctttccc

SEQ ID NO: 724 - DNA, 26 bp, Homo sapiens
agagtgagtacagcgaaggcggcctc

SEQ ID NO: 725 - DNA, 23 bp, Homo sapiens
tggacgccagaaagggaacccca

SEQ ID NO: 726 - DNA, 23 bp, Homo sapiens
agaatcactcagccgagcccttc

SEQ ID NO: 727 - DNA, 24 bp, Homo sapiens
gcatcccactcagtcagtctgggc

SEQ ID NO: 728 - DNA, 24 bp, Homo sapiens
aactatgcccatcgtattttacca

SEQ ID NO: 729 - DNA, 26 bp, Homo sapiens
ggataaagcacaggatgccccagtta

SEQ ID NO: 730 - DNA, 26 bp, Homo sapiens
gtcacacaacagagacagacttgata

SEQ ID NO: 731 - DNA, 25 bp, Homo sapiens
ggttcaagcaattctcctgcctcag

SEQ ID NO: 732 - DNA, 25 bp, Homo sapiens
tcctgggttcaagtgagtctcctgc

SEQ ID NO: 733 - DNA, 23 bp, Homo sapiens
cgggatgcctgctgagttgccat

SEQ ID NO: 734 - DNA, 23 bp, Homo sapiens
ccgctgaggtctaagggtttcca

SEQ ID NO: 735 - DNA, 22 bp, Homo sapiens
ggcgtggtctcggctcactgca

SEQ ID NO: 736 - DNA, 22 bp, Homo sapiens
cggctcactgcaacctccgcct

SEQ ID NO: 737 - DNA, 23 bp, Homo sapiens
gcatcaaacagggaggtgccagc

SEQ ID NO: 738 - DNA, 23 bp, Homo sapiens
cctcaggacagccagtgaaggat

SEQ ID NO: 739 - DNA, 23 bp, Homo sapiens
tgtccctctcttgctctcctgcc

SEQ ID NO: 740 - DNA, 23 bp, Homo sapiens
ctctgatggctgtgctgaggaca

SEQ ID NO: 741 - DNA, 25 bp, Homo sapiens
cccccgagaaacactcacatcttgg

SEQ ID NO: 742 - DNA, 25 bp, Homo sapiens
ccccggggctcctgcccggacggcg

SEQ ID NO: 743 - DNA, 26 bp, Homo sapiens
tctcagtaacttacattttcagtttg

SEQ ID NO: 744 - DNA, 26 bp, Homo sapiens
aatcccaacactttgggaggccgagg

SEQ ID NO: 745 - DNA, 24 bp, Homo sapiens
ctaaaatggcaagtttagaagggc

SEQ ID NO: 746 - DNA, 24 bp, Homo sapiens
gtctgccagaaaagccagccaagg

SEQ ID NO: 747 - DNA, 26 bp, Homo sapiens
cacttggtcacctcactattcaatgc

SEQ ID NO: 748 - DNA, 24 bp, Homo sapiens
cggttcgctttcccctggttatgc

SEQ ID NO: 749 - DNA, 25 bp, Homo sapiens
ttctgatcttagaggaaatgctttc

SEQ ID NO: 750 - DNA, 25 bp, Homo sapiens
gacgcggcggcaaggcctcggggga

SEQ ID NO: 751 - DNA, 26 bp, Homo sapiens
cctcagccttctgagtagcagggatt

SEQ ID NO: 752 - DNA, 26 bp, Homo sapiens
ttcttttgacagaatctcactcttac

SEQ ID NO: 753 - DNA, 25 bp, Homo sapiens
accaatgaaggtggccacactaaac

SEQ ID NO: 754 - DNA, 25 bp, Homo sapiens
ccagaggaaccccgggctcttggct

SEQ ID NO: 755 - DNA, 28 bp, Homo sapiens
ggaggaggctggtggagaccctgttgga

SEQ ID NO: 756 - DNA, 23 bp, Homo sapiens
gtgtgatttcaggggtgcctgcc

SEQ ID NO: 757 - DNA, 28 bp, Homo sapiens
tgaagaaacaaagcgaggcaaggtgacg

SEQ ID NO: 758 - DNA, 21 bp, Homo sapiens
gatcttggctcactgcaacct

SEQ ID NO: 759 - DNA, 28 bp, Homo sapiens
tcgcccctcgctcattcctcccgaccaa

SEQ ID NO: 760 - DNA, 28 bp, Homo sapiens
tcatcatacttctccctttccaatctga

SEQ ID NO: 761 - DNA, 28 bp, Homo sapiens
ggcaaaggtcaggtgggtgaggaagccc

SEQ ID NO: 762 - DNA, 28 bp, Homo sapiens
ggagggcagcactgatttgttcgtggtc

SEQ ID NO: 763 - DNA, 26 bp, Homo sapiens
ggcctcccgagtagctgggattacaa

SEQ ID NO: 764 - DNA, 22 bp, Homo sapiens
tgcctcagcctcccgagtagct

SEQ ID NO: 765 - DNA, 26 bp, Homo sapiens
ttcaagcgattcttctgcctcagcct

SEQ ID NO: 766 - DNA, 23 bp, Homo sapiens
gcgggcggcgaagtaaaggccca

SEQ ID NO: 767 - DNA, 22 bp, Homo sapiens
tctccttcctcagcctcctgag

SEQ ID NO: 768 - DNA, 28 bp, Homo sapiens
gctgtttgccacccaagagtgatgcggg

SEQ ID NO: 769 - DNA, 26 bp, Homo sapiens
ataacagaagggaacagaagccaccc

SEQ ID NO: 770 - DNA, 27 bp, Homo sapiens
cacacgcatccctgctcccattctgag

SEQ ID NO: 771 - DNA, 25 bp, Homo sapiens
tataaaaaggggtcctaaaatagtt

SEQ ID NO: 772 - DNA, 26 bp, Homo sapiens
caattctcccatctcagcctcccgag

SEQ ID NO: 773 - DNA, 28 bp, Homo sapiens
ccagagtggacacatccgtatgagagcc

SEQ ID NO: 774 - DNA, 21 bp, Homo sapiens
aatacagacccgattttgcct

SEQ ID NO: 775 - DNA, 21 bp, Homo sapiens
tcttctgcctcggcctctgga

SEQ ID NO: 776 - DNA, 27 bp, Homo sapiens
cacacgcatccctgctcccattctgag

SEQ ID NO: 777 - DNA, 24 bp, Homo sapiens
gtacggggccggtctccccgcccg

SEQ ID NO: 778 - DNA, 22 bp, Homo sapiens
aattaatcttcattaagtcatg

SEQ ID NO: 779 - DNA, 26 bp, Homo sapiens
agatcctgtgaaaagataagccagtt

SEQ ID NO: 780 - DNA, 28 bp, Homo sapiens
cccacctcctaaccatcacctcctctcc

SEQ ID NO: 781 - DNA, 28 bp, Homo sapiens
ctttgcgtgtctgtggctctgtggctcc

SEQ ID NO: 782 - DNA, 25 bp, Homo sapiens
tttcgctctttttgcccaggctgga

SEQ ID NO: 783 - DNA, 28 bp, Homo sapiens
gctattgagtttgtagtctgaatgacat

SEQ ID NO: 784 - DNA, 28 bp, Homo sapiens
tgccgtccctttctggtaatcggtggaa

SEQ ID NO: 785 - DNA, 28 bp, Homo sapiens
ctgacgggcattcattgggcacctgctg

SEQ ID NO: 786 - DNA, 23 bp, Homo sapiens
ctgggtatggagtgcatacctat

SEQ ID NO: 787 - DNA, 27 bp, Homo sapiens
gcttgcttgaaggcggattcctccagg

SEQ ID NO: 788 - DNA, 27 bp, Homo sapiens
gacagtccagagcactcccaggcgtgg

SEQ ID NO: 789 - DNA, 28 bp, Homo sapiens
tacagggcaggaggcagcgtgtccagag

SEQ ID NO: 790 - DNA, 26 bp, Homo sapiens
tccctctctttccctctccctctctc

SEQ ID NO: 791 - DNA, 27 bp, Homo sapiens
actgcgggtctgggagtctctgggctt

SEQ ID NO: 792 - DNA, 26 bp, Homo sapiens
acaggccttactctaataccaagcgc

SEQ ID NO: 793 - DNA, 28 bp, Homo sapiens
gatttctggataaagcacaggatgcccc

SEQ ID NO: 794 - DNA, 24 bp, Homo sapiens
tctggggttcaagcaattctcctg

SEQ ID NO: 795 - DNA, 28 bp, Homo sapiens
cgggatgcctgctgagttgccatttagt

SEQ ID NO: 796 - DNA, 25 bp, Homo sapiens
caacctccacctcctgggttcaagt

SEQ ID NO: 797 - DNA, 28 bp, Homo sapiens
ggcttgggatgggaagtgggagaaggtg

SEQ ID NO: 798 - DNA, 24 bp, Homo sapiens
cgctgccgccatccaccgctgggt

SEQ ID NO: 799 - DNA, 22 bp, Homo sapiens
taatgaggtcaccctccttgaa

SEQ ID NO: 800 - DNA, 23 bp, Homo sapiens
gaccttctcagtaacttacattt

SEQ ID NO: 801 - DNA, 21 bp, Homo sapiens
agacagggtgacaggtgtgca

SEQ ID NO: 802 - DNA, 27 bp, Homo sapiens
gtggagagtctgttgtgaggcaggcac

SEQ ID NO: 803 - DNA, 24 bp, Homo sapiens
atttctgatcttagaggaaatgct

SEQ ID NO: 804 - DNA, 23 bp, Homo sapiens
atcttggctcacagcagtctctg

SEQ ID NO: 805 - DNA, 21 bp, Homo sapiens
gtccccatcacataaaagtac

SEQ ID NO: 806 - DNA, 23 bp, Homo sapiens
aagagcagggctggaggcaggcg

SEQ ID NO: 807 - DNA, 23 bp, Homo sapiens
gagctcgcggtgtggctcagccc

SEQ ID NO: 808 - DNA, 23 bp, Homo sapiens
gccgccctcctctcgctgccact

SEQ ID NO: 809 - DNA, 21 bp, Homo sapiens
ctcttgttgcctaggctggag

SEQ ID NO: 810 - DNA, 27 bp, Homo sapiens
ggaacctgaagacagagtgagccagcc

SEQ ID NO: 811 - DNA, 28 bp, Homo sapiens
ttctgccagccctaatccctctgaatac

SEQ ID NO: 812 - DNA, 23 bp, Homo sapiens
ccagccccgctcccagagatgga

SEQ ID NO: 813 - DNA, 28 bp, Homo sapiens
ggctatctggacctggattttgagggac

SEQ ID NO: 814 - DNA, 26 bp, Homo sapiens
gcagtggggcaatcttggcttactgc

SEQ ID NO: 815 - DNA, 22 bp, Homo sapiens
cgcctcctgggttcaagtgatt

SEQ ID NO: 816 - DNA, 26 bp, Homo sapiens
ctcttattgcccaggctggagtacag

SEQ ID NO: 817 - DNA, 23 bp, Homo sapiens
gaagacatgagtcactagtaagg

SEQ ID NO: 818 - DNA, 22 bp, Homo sapiens
atatcagctcactgcaacctct

SEQ ID NO: 819 - DNA, 27 bp, Homo sapiens
ccagatgcgacttggctgaccaccacc

SEQ ID NO: 820 - DNA, 28 bp, Homo sapiens
gcttttctttgtttggtagggttatgat

SEQ ID NO: 821 - DNA, 28 bp, Homo sapiens
cagaaggtgacagaagacccagaaaagg

SEQ ID NO: 822 - DNA, 25 bp, Homo sapiens
ggaggaaggtaggtgccaggctgag

SEQ ID NO: 823 - DNA, 26 bp, Homo sapiens
agctcactgtaacctctgcctcctgg

SEQ ID NO: 824 - DNA, 28 bp, Homo sapiens
gcttgtgtggaacagccaggcaccattg

SEQ ID NO: 825 - DNA, 21 bp, Homo sapiens
cacagacaccgaccggaacgg

SEQ ID NO: 826 - DNA, 21 bp, Homo sapiens
gtgtggtggcgccatctcagc

SEQ ID NO: 827 - DNA, 28 bp, Homo sapiens
gttacactgtctcacatttctccctggc

SEQ ID NO: 828 - DNA, 24 bp, Homo sapiens
tggaaaccaaagactcccttcccc

SEQ ID NO: 829 - DNA, 22 bp, Homo sapiens
gagttcaagaccagcctgacca

SEQ ID NO: 830 - DNA, 26 bp, Homo sapiens
tgtggcggctcatgcctggaatccca

SEQ ID NO: 831 - DNA, 28 bp, Homo sapiens
gtcacccaaagtccccacctgctcatac

SEQ ID NO: 832 - DNA, 28 bp, Homo sapiens
acggagaacttgctcaacacagaggtgc

SEQ ID NO: 833 - DNA, 25 bp, Homo sapiens
cctgggttcaagcgattctcctgcc

SEQ ID NO: 834 - DNA, 28 bp, Homo sapiens
cagcagtgggttctcaaatgttagcatc

SEQ ID NO: 835 - DNA, 28 bp, Homo sapiens
gtcttcatttccatttcctgacacacag

SEQ ID NO: 836 - DNA, 27 bp, Homo sapiens
aggagtccagtcaggttccagcccagg

SEQ ID NO: 837 - DNA, 23 bp, Homo sapiens
ttatgtaaaagtttctagaaatt

SEQ ID NO: 838 - DNA, 24 bp, Homo sapiens
cgtgggttgcgtcagtcccgtgta

SEQ ID NO: 839 - DNA, 28 bp, Homo sapiens
gggcagagggaggacacccattagaaat

SEQ ID NO: 840 - DNA, 27 bp, Homo sapiens
ggcttctccctgtttctgtcctcctcc

SEQ ID NO: 841 - DNA, 26 bp, Homo sapiens
aactccagacttgcgggaaaggcctg

SEQ ID NO: 842 - DNA, 28 bp, Homo sapiens
gcaagaatcactcagccgagcccttccc

SEQ ID NO: 843 - DNA, 26 bp, Homo sapiens
gcaaactatgcccatcgtattttacc

SEQ ID NO: 844 - DNA, 28 bp, Homo sapiens
gtcacacaacagagacagacttgataat

SEQ ID NO: 845 - DNA, 24 bp, Homo sapiens
gggttcaagtgagtctcctgcctc

SEQ ID NO: 846 - DNA, 28 bp, Homo sapiens
ccttcccttctggctccctctttacccc

SEQ ID NO: 847 - DNA, 25 bp, Homo sapiens
cctttccccaccaggttagggcttt

SEQ ID NO: 848 - DNA, 27 bp, Homo sapiens
tctgggctgggctgggactgcttggat

SEQ ID NO: 849 - DNA, 28 bp, Homo sapiens
caggagtggtcaatgtgtgccttgttgc

SEQ ID NO: 850 - DNA, 22 bp, Homo sapiens
ctgcccggacggcggccgccgc

SEQ ID NO: 851 - DNA, 23 bp, Homo sapiens
aggccaggagttcaagaccctag

SEQ ID NO: 852 - DNA, 21 bp, Homo sapiens
ggccaatgccaccccaatctc

SEQ ID NO: 853 - DNA, 28 bp, Homo sapiens
ggagagagccagtggtgagaaacgcagc

SEQ ID NO: 854 - DNA, 24 bp, Homo sapiens
gccccgcgcgtctcctctgccgcc

SEQ ID NO: 855 - DNA, 23 bp, Homo sapiens
tttttttgttttcttttgacaga

SEQ ID NO: 856 - DNA, 21 bp, Homo sapiens
gggctcttggctcacttggga

SEQ ID NO: 857 - DNA, 29 bp, Homo sapiens
taggctggtctcgagggaactccctcccc

SEQ ID NO: 858 - DNA, 40 bp, Homo sapiens
tgagtgactctagcgatgtcgactgcccgcccagatcccg

SEQ ID NO: 859 - DNA, 32 bp, Homo sapiens
aggagctgcaactttccatcgagttcgccctc

SEQ ID NO: 860 - DNA, 40 bp, Homo sapiens
atgttggtcaggctggtctcgaggccagcctgaccaacat

SEQ ID NO: 861 - DNA, 32 bp, Homo sapiens
agagggcgcggcctacactcgatcttatcctt

SEQ ID NO: 862 - DNA, 37 bp, Homo sapiens
tccttttgtcgaactcctaaccttgtgatctacccgc

SEQ ID NO: 863 - DNA, 32 bp, Homo sapiens
tgcgtagtcgagccagcatgagctctctgttc

SEQ ID NO: 864 - DNA, 36 bp, Homo sapiens
tcattctcgatgtacttctctgcctcagcctacgtg

SEQ ID NO: 865 - DNA, 40 bp, Homo sapiens
gtgttggccaggctggtctcgagacgaccctggccaacaa

SEQ ID NO: 866 - DNA, 40 bp, Homo sapiens
atgttggccaagctggtctcgagaccagccttgccaacat

SEQ ID NO: 867 - DNA, 40 bp, Homo sapiens
atgttggccaggctggtctcgagaccagcctgaccaacag

SEQ ID NO: 868 - DNA, 40 bp, Homo sapiens
ttctgtcttttcgctggctcgaacaacaaattgttaagtg

SEQ ID NO: 869 - DNA, 40 bp, Homo sapiens
atgttggccagactgatctcgagacgatcccggccaacat

SEQ ID NO: 870 - DNA, 32 bp, Homo sapiens
aggcagacagggaacttggcatatcgaggtgg

SEQ ID NO: 871 - DNA, 38 bp, Homo sapiens
tggtggacactactttaatcgaactattgggctcaagc

SEQ ID NO: 872 - DNA, 37 bp, Homo sapiens
tggacataactgtattctctctctctctctcgaagct

SEQ ID NO: 873 - DNA, 40 bp, Homo sapiens
aaactctaaacaggtctttcgagaaatgccacgctcacta

SEQ ID NO: 874 - DNA, 40 bp, Homo sapiens
atgttggccaggctggtctcgagaccagactggccaacat

SEQ ID NO: 875 - DNA, 35 bp, Homo sapiens
agtcacagcaaatgccacagggtgtgttcgaggaa

SEQ ID NO: 876 - DNA, 40 bp, Homo sapiens
aaaaaatcaatttagatctcgaagctgtcatctgtgggcg

SEQ ID NO: 877 - DNA, 40 bp, Homo sapiens
gtgttggtcaggctggtctcgagacaagcctgactaacat

SEQ ID NO: 878 - DNA, 34 bp, Homo sapiens
tctctctctctctctcgagcaggatgtggaacag

SEQ ID NO: 879 - DNA, 40 bp, Homo sapiens
ttctgtcttttcgctggctcgagataagtggacccagctg

SEQ ID NO: 880 - DNA, 40 bp, Homo sapiens
aactaagctgacttatattcgagatggagttttgctcttt

SEQ ID NO: 881 - DNA, 40 bp, Homo sapiens
ccacctgaggtcaggagttcgaactcctggactcaagtga

SEQ ID NO: 882 - DNA, 33 bp, Homo sapiens
agcagcaatggcagtggtggtgatcgaaatgcc

SEQ ID NO: 883 - DNA, 32 bp, Homo sapiens
atagtgatcgacttgggagtgcctgcgttccc

SEQ ID NO: 884 - DNA, 40 bp, Homo sapiens
atgttggtcaggctggtctcgagaccagtctgaccaacat

SEQ ID NO: 885 - DNA, 38 bp, Homo sapiens
acttaattttcgatacttcccctgggtgactctgacag

SEQ ID NO: 886 - DNA, 33 bp, Homo sapiens
acacaactacaaggtgatgagattatcgatggg

SEQ ID NO: 887 - DNA, 28 bp, Homo sapiens
tggtgctcaggaggtgtcgagccgagga

SEQ ID NO: 888 - DNA, 40 bp, Homo sapiens
tcatttgaggtcaggagttcgaattcctgagctcaaatga

SEQ ID NO: 889 - DNA, 34 bp, Homo sapiens
accaccgttgggatgaaagatggcctcgaagttg

SEQ ID NO: 890 - DNA, 34 bp, Homo sapiens
tgccttcacccaaactatctgatccctcgaccct

SEQ ID NO: 891 - DNA, 32 bp, Homo sapiens
acgctcccctcgacgatgacaccataaccctg

SEQ ID NO: 892 - DNA, 40 bp, Homo sapiens
tctctctgtctctcagattcgagctcttcttcggcgtctg

SEQ ID NO: 893 - DNA, 29 bp, Homo sapiens
tggcagcgcggcggttggttcgagtatca

SEQ ID NO: 894 - DNA, 40 bp, Homo sapiens
gtgcccggttccacccactcgaagtgatggaactcatagg

SEQ ID NO: 895 - DNA, 32 bp, Homo sapiens
tctctctctctctcgaattcctgacctcggcc

SEQ ID NO: 896 - DNA, 40 bp, Homo sapiens
atgttggccaggctggtctcgagaccagtctggccaacat

SEQ ID NO: 897 - DNA, 35 bp, Homo sapiens
tgcgattgtcgaggcctaacaattttccagggtcc

SEQ ID NO: 898 - DNA, 40 bp, Homo sapiens
atgttggccaggctggtctcgagaccaacctggccaacat

SEQ ID NO: 899 - DNA, 36 bp, Homo sapiens
acgccaatggtctgagttaatcgaatgcatcctggc

SEQ ID NO: 900 - DNA, 32 bp, Homo sapiens
agcagaaggctccagctgtacgctcgattcct

SEQ ID NO: 901 - DNA, 40 bp, Homo sapiens
ggccggctgccatggagctcgaccgccagggcccgcgtcc

SEQ ID NO: 902 - DNA, 40 bp, Homo sapiens
taagtgtgtccacttatgtcgagcaatccttctgcctcag

SEQ ID NO: 903 - DNA, 40 bp, Homo sapiens
ttagatacatcaagtccctcgaggctgcagaaggctgggt

SEQ ID NO: 904 - DNA, 34 bp, Homo sapiens
ttagactcgatggttccctattaccctctgcccg

SEQ ID NO: 905 - DNA, 40 bp, Homo sapiens
tttttccatgagaaacattcgaggtgctcggcgggcagcg

SEQ ID NO: 906 - DNA, 40 bp, Homo sapiens
atgttggtcaggctggtctcgagaccagcctgagcaacat

SEQ ID NO: 907 - DNA, 40 bp, Homo sapiens
acagttacagaggagaagtcgacggagtcggttcagtttg

SEQ ID NO: 908 - DNA, 27 bp, Homo sapiens
agggagttccctcgagaccagcctagg

SEQ ID NO: 909 - DNA, 40 bp, Homo sapiens
cgggatctgggcgggcagtcgacatcgctagagtcactca

SEQ ID NO: 910 - DNA, 32 bp, Homo sapiens
agggagggcgaactcgatggaaagttgcagct

SEQ ID NO: 911 - DNA, 40 bp, Homo sapiens
atgttggtcaggctggcctcgagaccagcctgaccaacat

SEQ ID NO: 912 - DNA, 32 bp, Homo sapiens
aaggataagatcgagtgtaggccgcgccctct

SEQ ID NO: 913 - DNA, 37 bp, Homo sapiens
aggcgggtagatcacaaggttaggagttcgacaaaag

SEQ ID NO: 914 - DNA, 32 bp, Homo sapiens
aacagagagctcatgctggctcgactacgcac

SEQ ID NO: 915 - DNA, 37 bp, Homo sapiens
acgtaggctgaggcagagaagtacatcgagaatgatc

SEQ ID NO: 916 - DNA, 40 bp, Homo sapiens
ttgttggccagggtcgtctcgagaccagcctggccaacac

SEQ ID NO: 917 - DNA, 40 bp, Homo sapiens
atgttggcaaggctggtctcgagaccagcttggccaacat

SEQ ID NO: 918 - DNA, 40 bp, Homo sapiens
ctgttggtcaggctggtctcgagaccagcctggccaacat

SEQ ID NO: 919 - DNA, 40 bp, Homo sapiens
cacttaacaatttgttgttcgagccagcgaaaagacagaa

SEQ ID NO: 920 - DNA, 40 bp, Homo sapiens
atgttggccgggatcgtctcgagatcagtctggccaacat

SEQ ID NO: 921 - DNA, 32 bp, Homo sapiens
accacctcgatatgccaagttccctgtctgcc

SEQ ID NO: 922 - DNA, 38 bp, Homo sapiens
tgcttgagcccaatagttcgattaaagtagtgtccacc

SEQ ID NO: 923 - DNA, 36 bp, Homo sapiens
agcttcgagagagagagagagaatacagttatgtcc

SEQ ID NO: 924 - DNA, 40 bp, Homo sapiens
tagtgagcgtggcatttctcgaaagacctgtttagagttt

SEQ ID NO: 925 - DNA, 40 bp, Homo sapiens
atgttggccagtctggtctcgagaccagcctggccaacat

SEQ ID NO: 926 - DNA, 34 bp, Homo sapiens
ttcctcgaacacaccctgtggcatttgctgtgac

SEQ ID NO: 927 - DNA, 40 bp, Homo sapiens
cgcccacagatgacagcttcgagatctaaattgatttttt

SEQ ID NO: 928 - DNA, 40 bp, Homo sapiens
atgttagtcaggcttgtctcgagaccagcctgaccaacac

SEQ ID NO: 929 - DNA, 34 bp, Homo sapiens
tctgttccacatcctgctcgagagagagagagag

SEQ ID NO: 930 - DNA, 40 bp, Homo sapiens
cagctgggtccacttatctcgagccagcgaaaagacagaa

SEQ ID NO: 931 - DNA, 40 bp, Homo sapiens
aaagagcaaaactccatctcgaatataagtcagcttagtt

SEQ ID NO: 932 - DNA, 40 bp, Homo sapiens
tcacttgagtccaggagttcgaactcctgacctcaggtgg

SEQ ID NO: 933 - DNA, 33 bp, Homo sapiens
tgggcatttcgatcaccaccactgccattgctg

SEQ ID NO: 934 - DNA, 35 bp, Homo sapiens
acgcaggcactcccaagtcgatcactatttggttc

SEQ ID NO: 935 - DNA, 40 bp, Homo sapiens
atgttggtcagactggtctcgagaccagcctgaccaacat

SEQ ID NO: 936 - DNA, 38 bp, Homo sapiens
ctgtcagagtcacccaggggaagtatcgaaaattaagt

SEQ ID NO: 937 - DNA, 37 bp, Homo sapiens
tccttggactctccccatcgataatctcatcaccttg

SEQ ID NO: 938 - DNA, 28 bp, Homo sapiens
tcctcggctcgacacctcctgagcacca

SEQ ID NO: 939 - DNA, 40 bp, Homo sapiens
tcatttgagctcaggaattcgaactcctgacctcaaatga

SEQ ID NO: 940 - DNA, 34 bp, Homo sapiens
aacttcgaggccatctttcatcccaacggtggtg

SEQ ID NO: 941 - DNA, 33 bp, Homo sapiens
agggtcgagggatcagatagtttgggtgaaggc

SEQ ID NO: 942 - DNA, 32 bp, Homo sapiens
cagggttatggtgtcatcgtcgaggggagcgt

SEQ ID NO: 943 - DNA, 40 bp, Homo sapiens
cagacgccgaagaagagctcgaatctgagagacagagaga

SEQ ID NO: 944 - DNA, 32 bp, Homo sapiens
ttatctgatactcgaaccaaccgccgcgctgc

SEQ ID NO: 945 - DNA, 40 bp, Homo sapiens
cctatgagttccatcacttcgagtgggtggaaccgggcac

SEQ ID NO: 946 - DNA, 32 bp, Homo sapiens
aggccgaggtcaggaattcgagagagagagag

SEQ ID NO: 947 - DNA, 40 bp, Homo sapiens
atgttggccagactggtctcgagaccagcctggccaacat

SEQ ID NO: 948 - DNA, 35 bp, Homo sapiens
accctggaaaattgttaggcctcgacaatcgcacc

SEQ ID NO: 949 - DNA, 40 bp, Homo sapiens
atgttggccaggttggtctcgagaccagcctggccaacat

SEQ ID NO: 950 - DNA, 36 bp, Homo sapiens
tggccaggatgcattcgattaactcagaccattggc

SEQ ID NO: 951 - DNA, 32 bp, Homo sapiens
aggaatcgagcgtacagctggagccttctgct

SEQ ID NO: 952 - DNA, 40 bp, Homo sapiens
ggacgcgggccctggcggtcgagctccatggcagccggcc

SEQ ID NO: 953 - DNA, 40 bp, Homo sapiens
ctgaggcagaaggattgctcgacataagtggacacactta

SEQ ID NO: 954 - DNA, 40 bp, Homo sapiens
acccagccttctgcagcctcgagggacttgatgtatctaa

SEQ ID NO: 955 - DNA, 36 bp, Homo sapiens
tgcgggcagagggtaatagggaaccatcgagtctaa

SEQ ID NO: 956 - DNA, 40 bp, Homo sapiens
cgctgcccgccgagcacctcgaatgtttctcatggaaaaa

SEQ ID NO: 957 - DNA, 40 bp, Homo sapiens
atgttgctcaggctggtctcgagaccagcctgaccaacat

SEQ ID NO: 958 - DNA, 40 bp, Homo sapiens
caaactgaaccgactccgtcgacttctcctctgtaactgt

SEQ ID NO: 959 - DNA, 60 bp, Homo sapiens
atgggcctcagaggggaagaagtacatatcgagaatgggcatctctacccttgttaggtc

SEQ ID NO: 960 - DNA, 60 bp, Homo sapiens
actgcgcctggcatatttatttatttattcgagccgcttgagcatagagcttatggagat

SEQ ID NO: 961 - DNA, 60 bp, Homo sapiens
tagtttccaagtgtgtgatagctccagatcgagtctgaggagtgagcgggaagctgcaca

SEQ ID NO: 962 - DNA, 60 bp, Homo sapiens
tttctctttctctctctctttttttttttcgagatccttaggggaccttgtagtaaaata

SEQ ID NO: 963 - DNA, 60 bp, Homo sapiens
gactccacatttccaccagctcaaacagtcgaacaattcttctgcctcagcctcccaagc

SEQ ID NO: 964 - DNA, 60 bp, Homo sapiens
tggcttcttcttaactttctcgggtgagtcgaagagcccaaataacttgttttagaccag

SEQ ID NO: 965 - DNA, 60 bp, Homo sapiens
tggtttcagaagaggggcccagggaacctcgaatcttctgagggagcgtggcccaggtga

SEQ ID NO: 966 - DNA, 60 bp, Homo sapiens
catctgtcagagtcacccaggggaagtatcgaaggttttttgcagaacaacataccaaat

SEQ ID NO: 967 - DNA, 60 bp, Homo sapiens
ggctgaggtgagagaatcacctgagccttcgagttgcaaatttctgttttcttttttttt

SEQ ID NO: 968 - DNA, 60 bp, Homo sapiens
tgatagccaagacaatggggaaaaggcctcgacccttgcacgggcgcagatccaggtgca

SEQ ID NO: 969 - DNA, 60 bp, Homo sapiens
aggcttcagctggggcaggcttggcctgtcgacaccaatgtcttggatgtttaacctcca

SEQ ID NO: 970 - DNA, 60 bp, Homo sapiens
catcatggcagatgggttatccaactcctcgaaagctttcttttctaagccttaggatgc

SEQ ID NO: 971 - DNA, 60 bp, Homo sapiens
aggtttcaccatgttggccaggatggtctcgagatcagcctggtcaacatggtgaaaccc

SEQ ID NO: 972 - DNA, 60 bp, Homo sapiens
ccaccatgccaggcacgaacatggtcactcgaaattacgtgagccttagttctttttatt

SEQ ID NO: 973 - DNA, 60 bp, Homo sapiens
cacaccaccgttgggatgaaagatggcctcgatacatgcttgtcagtgaaagagcgatgc

SEQ ID NO: 974 - DNA, 60 bp, Homo sapiens
tcattggtaggacatctcaaaatggcactcgagctctacagccagtacctggagcgcctg

SEQ ID NO: 975 - DNA, 60 bp, Homo sapiens
tgtgctgggggcactagtcccgcttgcttcgatgcctgggctccttctgccccatcccca

SEQ ID NO: 976 - DNA, 60 bp, Homo sapiens
gctgggctccccgggctcagcgtccttgtcgaaaccgaaagcaaagaggaagcgggatct

SEQ ID NO: 977 - DNA, 60 bp, Homo sapiens
catctgtcagagtcacccaggggaagtatcgattgtgctttcagtgcctcagtgagatgc

SEQ ID NO: 978 - DNA, 60 bp, Homo sapiens
gggtttcaccatgttggccaggctggtctcgagaccagcctgggccaacatggggaaacc

SEQ ID NO: 979 - DNA, 60 bp, Homo sapiens
gggctcacttcatatcctctgcccatcttcgatggttttccatgacgattgtccagttta

SEQ ID NO: 980 - DNA, 60 bp, Homo sapiens
ctcatacatcacagccccagcctcggaatcgacctcagcttcattcttcttaaacaagag

SEQ ID NO: 981 - DNA, 60 bp, Homo sapiens
accaggagcagcctctgctctgcccacctcgaagcttctcaaacgccactctaacgaccc

SEQ ID NO: 982 - DNA, 60 bp, Homo sapiens
tgataaggccatgttttggagacagtgatcgagcatttcttaaattccctcagaatttgc

SEQ ID NO: 983 - DNA, 60 bp, Homo sapiens
cggggccctttgctgtgagtagaggatgtcgagggctgggcggcagcctccctgggctcc

SEQ ID NO: 984 - DNA, 60 bp, Homo sapiens
gctctgacataccaccctgccaagtacctcgactcgggttcttccctgcaggtggcgtgg

SEQ ID NO: 985 - DNA, 60 bp, Homo sapiens
gggtttcaccatgttgaccaggctggtctcgagaccagcctggcaaacatggtgaaaccc

SEQ ID NO: 986 - DNA, 60 bp, Homo sapiens
catctgtcagagtcacccaggggaagtatcgactagacacttccaaaatactaaccatac

SEQ ID NO: 987 - DNA, 60 bp, Homo sapiens
gatatttgtctagctttgaggatttcgttcgaattttaagctccacaaatgaccaagaac

SEQ ID NO: 988 - DNA, 60 bp, Homo sapiens
ccctaaatggccacgaggttccccggcctcgaggtgtcagatatttttcttcctgtatat

SEQ ID NO: 989 - DNA, 60 bp, Homo sapiens
actctttccattatgtgcaattgtaacttcgatgccagagagggagctcgccctgggaat

SEQ ID NO: 990 - DNA, 60 bp, Homo sapiens
gaggcaggtggaatgggtggagtccaagtcgactccaaaaccctaaaacaggcgctggag

SEQ ID NO: 991 - DNA, 60 bp, Homo sapiens
tgataaggccatgttttggagacagtgatcgaagtttgtgctctttgtgattcagacaga

SEQ ID NO: 992 - DNA, 60 bp, Homo sapiens
cccctaggcctgggaatagggaaacggttcgatttggtttcccaaggtcttatcagtgat

SEQ ID NO: 993 - DNA, 60 bp, Homo sapiens
ttgagggcatttggctgctcttttacactcgagccagcgaaaagacagaaccggaagaga

SEQ ID NO: 994 - DNA, 60 bp, Homo sapiens
agactgggtgacagagtgagatcgtgtctcgattttccagcagtgtgcctggaggagttt

SEQ ID NO: 995 - DNA, 60 bp, Homo sapiens
gagtggagaaaacggttggatgagggactcgagtatagcattcaattgctctttcatatt

SEQ ID NO: 996 - DNA, 60 bp, Homo sapiens
tagggtcttcttccagagaatcaaattctcgaccagcttggccccatctaaagtatgatt

SEQ ID NO: 997 - DNA, 60 bp, Homo sapiens
tttccttctgagaagagatattccttcatcgactggtcacgatgggctgcttcatctgtg

SEQ ID NO: 998 - DNA, 60 bp, Homo sapiens
tgataaggccatgttttggagacagtgatcgacagatctccattatttttcagccggctc

SEQ ID NO: 999 - DNA, 60 bp, Homo sapiens
cacaccaccgttgggatgaaagatggcctcgagctggtcatgacacgctttaagaccctg

SEQ ID NO: 1000 - DNA, 60 bp, Homo sapiens
gggtttcaccaggttggccatgctgggttcgagagcagcctgggcaacacagtgagacct

SEQ ID NO: 1001 - DNA, 60 bp, Homo sapiens
tcttgctttcttgctttctttcttgctttcgataacctgaaataccgacttttttggatt

SEQ ID NO: 1002 - DNA, 60 bp, Homo sapiens
tgatgttatggaaaaccatttggccttttcgaatcttctgagggagcgtggcccaggtga

SEQ ID NO: 1003 - DNA, 60 bp, Homo sapiens
ggacataactgtattctctctctctctctcgaactcctgacttcggctgatctgcccacc

SEQ ID NO: 1004 - DNA, 60 bp, Homo sapiens
aaacaggcactgcgtgttcccagcaccctcgattccatcttccgacctgccagcatcatg

SEQ ID NO: 1005 - DNA, 60 bp, Homo sapiens
ctcttttctcttgcagaggtggcaaaactcgaaagaacagcagctcgcgacctgcggggc

SEQ ID NO: 1006 - DNA, 60 bp, Homo sapiens
gttattattcttccatttatatttttgttcgacccttctgctttctctccaggggatggc

SEQ ID NO: 1007 - DNA, 60 bp, Homo sapiens
gtactgaaggtctagcatatgggtagagtcgacctctctgggctcaggtgatcctcccac

SEQ ID NO: 1008 - DNA, 26 bp, Homo sapiens
tgtgctgatgtctgctgctctcaaca

SEQ ID NO: 1009 - DNA, 26 bp, Homo sapiens
gataaaaccctgattctctctcctgg

SEQ ID NO: 1010 - DNA, 23 bp, Homo sapiens
cccgtgctgagatggagaacctg

SEQ ID NO: 1011 - DNA, 23 bp, Homo sapiens
ggtttcactcctcccacctcatc

SEQ ID NO: 1012 - DNA, 26 bp, Homo sapiens
ggagagtttctgttcaaagggtgtag

SEQ ID NO: 1013 - DNA, 26 bp, Homo sapiens
cccacaaagggtgttcaatcatccac

SEQ ID NO: 1014 - DNA, 26 bp, Homo sapiens
aggaaataagccctacgtggcaggag

SEQ ID NO: 1015 - DNA, 26 bp, Homo sapiens
caggtttctcacaaacagactgaaaa

SEQ ID NO: 1016 - DNA, 25 bp, Homo sapiens
gggagggagagattggttctttacc

SEQ ID NO: 1017 - DNA, 26 bp, Homo sapiens
acataggtgatggtggctgacttcct

SEQ ID NO: 1018 - DNA, 23 bp, Homo sapiens
aggagggcaaaggtcacagtggc

SEQ ID NO: 1019 - DNA, 23 bp, Homo sapiens
caccttgctgcttgccaacagtg

SEQ ID NO: 1020 - DNA, 22 bp, Homo sapiens
atttagcaagacattggaagaa

SEQ ID NO: 1021 - DNA, 22 bp, Homo sapiens
ttgtgcctccgtatactataat

SEQ ID NO: 1022 - DNA, 26 bp, Homo sapiens
cagcagtgggttctcaaatgttagca

SEQ ID NO: 1023 - DNA, 26 bp, Homo sapiens
ctgtcatctaagcctccattcataac

SEQ ID NO: 1024 - DNA, 22 bp, Homo sapiens
caggcagatcacttgagcctag

SEQ ID NO: 1025 - DNA, 22 bp, Homo sapiens
aaatacaaaaattagcttggaa

SEQ ID NO: 1026 - DNA, 26 bp, Homo sapiens
agcctggtcaagtgaaaagcccattt

SEQ ID NO: 1027 - DNA, 26 bp, Homo sapiens
tgaggatgacccagcagatagggaga

SEQ ID NO: 1028 - DNA, 22 bp, Homo sapiens
agcggtgggagctgggggcgcg

SEQ ID NO: 1029 - DNA, 22 bp, Homo sapiens
aaggagtactagtagatacaag

SEQ ID NO: 1030 - DNA, 25 bp, Homo sapiens
acatactttctagatcaatttctta

SEQ ID NO: 1031 - DNA, 25 bp, Homo sapiens
tttcaaaaaccaactccattagcag

SEQ ID NO: 1032 - DNA, 23 bp, Homo sapiens
tgcaacatctacctcccgggttc

SEQ ID NO: 1033 - DNA, 23 bp, Homo sapiens
gaaacctccgcctcccaggttca

SEQ ID NO: 1034 - DNA, 23 bp, Homo sapiens
ccccagccctccttctcttcttt

SEQ ID NO: 1035 - DNA, 23 bp, Homo sapiens
aatcccagcactcaccacacggc

SEQ ID NO: 1036 - DNA, 23 bp, Homo sapiens
gcttgaaggcggattcctccagg

SEQ ID NO: 1037 - DNA, 23 bp, Homo sapiens
gctgcctgactactcctgggaaa

SEQ ID NO: 1038 - DNA, 24 bp, Homo sapiens
ggctgtgaggttgtcctttactgc

SEQ ID NO: 1039 - DNA, 23 bp, Homo sapiens
tcctgggagatgaagcgacgcag

SEQ ID NO: 1040 - DNA, 23 bp, Homo sapiens
tgggtctgtgcttgctgtgcggt

SEQ ID NO: 1041 - DNA, 23 bp, Homo sapiens
cctgcctgtctgtctggagaaga

SEQ ID NO: 1042 - DNA, 23 bp, Homo sapiens
tggggcaacctcaccagcttggt

SEQ ID NO: 1043 - DNA, 23 bp, Homo sapiens
aggcctttccgccgcccagagag

SEQ ID NO: 1044 - DNA, 26 bp, Homo sapiens
cagcagtgggttctcaaatgttagca

SEQ ID NO: 1045 - DNA, 25 bp, Homo sapiens
gctgtcttgctgctgatagccttta

SEQ ID NO: 1046 - DNA, 22 bp, Homo sapiens
agtgatctgtctgcctcggcct

SEQ ID NO: 1047 - DNA, 22 bp, Homo sapiens
gtaaagtggtatgatcttggct

SEQ ID NO: 1048 - DNA, 26 bp, Homo sapiens
gggacctttgaaccacatttggaggg

SEQ ID NO: 1049 - DNA, 26 bp, Homo sapiens
cagggatttctgaggacaagacatct

SEQ ID NO: 1050 - DNA, 25 bp, Homo sapiens
tcgggcgcttccggggagctaggcc

SEQ ID NO: 1051 - DNA, 25 bp, Homo sapiens
gggcaccaaaatgattaaggacatt

SEQ ID NO: 1052 - DNA, 23 bp, Homo sapiens
tgaccaagagtgagccaagcccc

SEQ ID NO: 1053 - DNA, 23 bp, Homo sapiens
gcccttactgactgccaggaaac

SEQ ID NO: 1054 - DNA, 25 bp, Homo sapiens
ctgaactgtcgtcactggtgctgtg

SEQ ID NO: 1055 - DNA, 26 bp, Homo sapiens
cctacaagccagaagggattgagaat

SEQ ID NO: 1056 - DNA, 23 bp, Homo sapiens
ctggaagagacagagcccacaca

SEQ ID NO: 1057 - DNA, 23 bp, Homo sapiens
cgcagcccttccaagcctcagaa

SEQ ID NO: 1058 - DNA, 25 bp, Homo sapiens
aaactaggaaaagagaaatagaaca

SEQ ID NO: 1059 - DNA, 25 bp, Homo sapiens
gctgtggcggtcccttggtggggaa

SEQ ID NO: 1060 - DNA, 21 bp, Homo sapiens
cagcctgtagcaattgtcctg

SEQ ID NO: 1061 - DNA, 21 bp, Homo sapiens
aggttcaagcgattctcctgc

SEQ ID NO: 1062 - DNA, 26 bp, Homo sapiens
cagcagtgggttctcaaatgttagca

SEQ ID NO: 1063 - DNA, 26 bp, Homo sapiens
gtcctctctacaagacaataggcagc

SEQ ID NO: 1064 - DNA, 23 bp, Homo sapiens
tagatagaagcaatgtcagaaac

SEQ ID NO: 1065 - DNA, 23 bp, Homo sapiens
atatcgttttcctgagtaatagt

SEQ ID NO: 1066 - DNA, 21 bp, Homo sapiens
agtccaaagcttcgggagaag

SEQ ID NO: 1067 - DNA, 21 bp, Homo sapiens
gtcaggaaaagagaggtaaat

SEQ ID NO: 1068 - DNA, 23 bp, Homo sapiens
tagagaagagaggactctcccta

SEQ ID NO: 1069 - DNA, 23 bp, Homo sapiens
ccgattagatacctgatcattta

SEQ ID NO: 1070 - DNA, 24 bp, Homo sapiens
tccacagggaaagagaatccctcg

SEQ ID NO: 1071 - DNA, 24 bp, Homo sapiens
cctgacgaagcaggggcggggcga

SEQ ID NO: 1072 - DNA, 21 bp, Homo sapiens
ggagggaggatgggggatgaa

SEQ ID NO: 1073 - DNA, 21 bp, Homo sapiens
aagtagcatgcacaggttcca

SEQ ID NO: 1074 - DNA, 26 bp, Homo sapiens
ttcattccagccctccacgccctaat

SEQ ID NO: 1075 - DNA, 26 bp, Homo sapiens
gccaatacattcaggaaacacaggca

SEQ ID NO: 1076 - DNA, 25 bp, Homo sapiens
gagtgctgaaacttccttttcttct

SEQ ID NO: 1077 - DNA, 25 bp, Homo sapiens
gggcatgggcccctcgtcccctcgt

SEQ ID NO: 1078 - DNA, 26 bp, Homo sapiens
gactgagcaacataaggagacccgtc

SEQ ID NO: 1079 - DNA, 26 bp, Homo sapiens
gagaaaggaaatgggacagaagcaat

SEQ ID NO: 1080 - DNA, 25 bp, Homo sapiens
gactctcaggcacagaggcaaaaaa

SEQ ID NO: 1081 - DNA, 25 bp, Homo sapiens
aacagattttaaaacgtgtctcatg

SEQ ID NO: 1082 - DNA, 26 bp, Homo sapiens
agtgtgtgcccaggtgagagaccatt

SEQ ID NO: 1083 - DNA, 26 bp, Homo sapiens
ggcagacgatacatttccagatagtt

SEQ ID NO: 1084 - DNA, 24 bp, Homo sapiens
aggttggtgtggcccctgaagaca

SEQ ID NO: 1085 - DNA, 24 bp, Homo sapiens
ctgtcccagaaaaaagaaaaatcc

SEQ ID NO: 1086 - DNA, 23 bp, Homo sapiens
gggaaggaggagtggaaacggaa

SEQ ID NO: 1087 - DNA, 23 bp, Homo sapiens
aagcggagcctggagcatctcgt

SEQ ID NO: 1088 - DNA, 23 bp, Homo sapiens
cttgaaggcggattcctccaggt

SEQ ID NO: 1089 - DNA, 23 bp, Homo sapiens
agcaggcacggaacacagtggtg

SEQ ID NO: 1090 - DNA, 25 bp, Homo sapiens
agctcactgcaacctctgcctcccc

SEQ ID NO: 1091 - DNA, 25 bp, Homo sapiens
gttctgctgggggcaggccagggga

SEQ ID NO: 1092 - DNA, 23 bp, Homo sapiens
ggtgccactgccttggttcctgt

SEQ ID NO: 1093 - DNA, 23 bp, Homo sapiens
ggtccctgccaaactggaaacga

SEQ ID NO: 1094 - DNA, 21 bp, Homo sapiens
cacaggttgaaagtgaaaatt

SEQ ID NO: 1095 - DNA, 21 bp, Homo sapiens
ggtgctgtaccaaagtaccac

SEQ ID NO: 1096 - DNA, 26 bp, Homo sapiens
ggataaagcacaggatgccccagtta

SEQ ID NO: 1097 - DNA, 24 bp, Homo sapiens
cggaagtcctacccaagcaatcat

SEQ ID NO: 1098 - DNA, 26 bp, Homo sapiens
gcttcctgagtcttcatcacaaaacc

SEQ ID NO: 1099 - DNA, 26 bp, Homo sapiens
gccactgtgctcaatctgcgttctga

SEQ ID NO: 1100 - DNA, 23 bp, Homo sapiens
ccaggttcagacacagtgcccac

SEQ ID NO: 1101 - DNA, 23 bp, Homo sapiens
tcggctacgctgccacttcaatg

SEQ ID NO: 1102 - DNA, 26 bp, Homo sapiens
cagactatgtgcctcaaagacagtgc

SEQ ID NO: 1103 - DNA, 26 bp, Homo sapiens
cagaagggtgaggacaaagagatagc

SEQ ID NO: 1104 - DNA, 22 bp, Homo sapiens
gccatgagaaacacaaacgact

SEQ ID NO: 1105 - DNA, 22 bp, Homo sapiens
ggtggctcacacctgtaatccc

SEQ ID NO: 1106 - DNA, 28 bp, Homo sapiens
cttcctgtcatctctgtggctctcagca

SEQ ID NO: 1107 - DNA, 28 bp, Homo sapiens
cggtccccgtgctgagatggagaacctg

SEQ ID NO: 1108 - DNA, 28 bp, Homo sapiens
ggagagtttctgttcaaagggtgtagag

SEQ ID NO: 1109 - DNA, 23 bp, Homo sapiens
cccctctcctactaggaaataag

SEQ ID NO: 1110 - DNA, 28 bp, Homo sapiens
gtgatgatggttctcctgctccgtgtcc

SEQ ID NO: 1111 - DNA, 24 bp, Homo sapiens
ggctgccccacggctacagaaggc

SEQ ID NO: 1112 - DNA, 21 bp, Homo sapiens
cccaaaggcattttggagaat

SEQ ID NO: 1113 - DNA, 28 bp, Homo sapiens
gacagcagtgggttctcaaatgttagca

SEQ ID NO: 1114 - DNA, 23 bp, Homo sapiens
gagcctaggagtttgagacgagt

SEQ ID NO: 1115 - DNA, 28 bp, Homo sapiens
gcagcaaagcattcaagagttacctggc

SEQ ID NO: 1116 - DNA, 21 bp, Homo sapiens
caggcgttccgagggccagag

SEQ ID NO: 1117 - DNA, 21 bp, Homo sapiens
tgtgaggacatagtaagtata

SEQ ID NO: 1118 - DNA, 21 bp, Homo sapiens
cccgggttcaggtgattctcc

SEQ ID NO: 1119 - DNA, 23 bp, Homo sapiens
cccctgcccttctgcctggtctc

SEQ ID NO: 1120 - DNA, 27 bp, Homo sapiens
gcttgcttgaaggcggattcctccagg

SEQ ID NO: 1121 - DNA, 25 bp, Homo sapiens
aggctgtgaggttgtcctttactgc

SEQ ID NO: 1122 - DNA, 28 bp, Homo sapiens
tcctgggtctgtgcttgctgtgcggtgg

SEQ ID NO: 1123 - DNA, 24 bp, Homo sapiens
acctcaccagcttggtctctttgt

SEQ ID NO: 1124 - DNA, 28 bp, Homo sapiens
gacagcagtgggttctcaaatgttagca

SEQ ID NO: 1125 - DNA, 26 bp, Homo sapiens
gacctcaagtgatctgtctgcctcgg

SEQ ID NO: 1126 - DNA, 25 bp, Homo sapiens
cattgcccctggtgcgacctgcccc

SEQ ID NO: 1127 - DNA, 21 bp, Homo sapiens
gcttccggggagctaggcctc

SEQ ID NO: 1128 - DNA, 28 bp, Homo sapiens
tgggtgcggcgacaggctcatctgcgat

SEQ ID NO: 1129 - DNA, 23 bp, Homo sapiens
cgtcactggtgctgtggtgcccg

SEQ ID NO: 1130 - DNA, 28 bp, Homo sapiens
acctggaagagacagagcccacacacgg

SEQ ID NO: 1131 - DNA, 24 bp, Homo sapiens
agtgtctcacaaaactcaaggaaa

SEQ ID NO: 1132 - DNA, 24 bp, Homo sapiens
gtcctgccttagcctcctgagtag

SEQ ID NO: 1133 - DNA, 28 bp, Homo sapiens
gacagcagtgggttctcaaatgttagca

SEQ ID NO: 1134 - DNA, 21 bp, Homo sapiens
aggactacggtgaaaaaggaa

SEQ ID NO: 1135 - DNA, 25 bp, Homo sapiens
agcggtccccctgagtccaaagctt

SEQ ID NO: 1136 - DNA, 24 bp, Homo sapiens
gaaatggtgctttggtgcttagat

SEQ ID NO: 1137 - DNA, 22 bp, Homo sapiens
gtctcattttctaccctccaca

SEQ ID NO: 1138 - DNA, 23 bp, Homo sapiens
gggccccaggtcagtgtgggctc

SEQ ID NO: 1139 - DNA, 23 bp, Homo sapiens
gcggtctgacagccccacctgcc

SEQ ID NO: 1140 - DNA, 26 bp, Homo sapiens
ggggctgggagagaggaagagattat

SEQ ID NO: 1141 - DNA, 26 bp, Homo sapiens
gccagggagttgggaagtgtgggaag

SEQ ID NO: 1142 - DNA, 23 bp, Homo sapiens
atttgttgagcatctactctttg

SEQ ID NO: 1143 - DNA, 26 bp, Homo sapiens
agtgtgtgcccaggtgagagaccatt

SEQ ID NO: 1144 - DNA, 24 bp, Homo sapiens
ggaggtacctcaacagctcctcag

SEQ ID NO: 1145 - DNA, 23 bp, Homo sapiens
cgtcactggtgctgtggtgcccg

SEQ ID NO: 1146 - DNA, 27 bp, Homo sapiens
gcttgcttgaaggcggattcctccagg

SEQ ID NO: 1147 - DNA, 21 bp, Homo sapiens
gatggagtctcactctgtcac

SEQ ID NO: 1148 - DNA, 28 bp, Homo sapiens
ctttggtgccactgccttggttcctgtt

SEQ ID NO: 1149 - DNA, 26 bp, Homo sapiens
aaaaaacacttccaaagcttttcacc

SEQ ID NO: 1150 - DNA, 27 bp, Homo sapiens
cacacgcatccctgctcccattctgag

SEQ ID NO: 1151 - DNA, 28 bp, Homo sapiens
gcagttggcttcagacgggcagacaggg

SEQ ID NO: 1152 - DNA, 28 bp, Homo sapiens
tgtccaggaacaggaagtttcagagggc

SEQ ID NO: 1153 - DNA, 28 bp, Homo sapiens
ctcagactatgtgcctcaaagacagtgc

SEQ ID NO: 1154 - DNA, 22 bp, Homo sapiens
caagagccaaaggatcaacacc

SEQ ID NO: 1155 - DNA, 24 bp, Homo sapiens
ctgctgctgcctccatcctcctcc

SEQ ID NO: 1156 - DNA, 23 bp, Homo sapiens
cgggcagagagccagggtgaagc

SEQ ID NO: 1157 - DNA, 25 bp, Homo sapiens
gccccttctccttcccaccattccc

SEQ ID NO: 1158 - DNA, 23 bp, Homo sapiens
ctcacaaacagactgaaaaaata

SEQ ID NO: 1159 - DNA, 27 bp, Homo sapiens
ctgctcctctgcctgccttgtagtctc

SEQ ID NO: 1160 - DNA, 27 bp, Homo sapiens
gttacaccttgctgcttgccaacagtg

SEQ ID NO: 1161 - DNA, 21 bp, Homo sapiens
attagttttccaggggtgctg

SEQ ID NO: 1162 - DNA, 28 bp, Homo sapiens
gctgtcatctaagcctccattcataact

SEQ ID NO: 1163 - DNA, 23 bp, Homo sapiens
taaaaatacaaaaattagcttgg

SEQ ID NO: 1164 - DNA, 26 bp, Homo sapiens
acttggtgaggctggaggaggagggc

SEQ ID NO: 1165 - DNA, 21 bp, Homo sapiens
ggagtactagtagatacaaga

SEQ ID NO: 1166 - DNA, 21 bp, Homo sapiens
gcaggtaggggtgtgggaggg

SEQ ID NO: 1167 - DNA, 21 bp, Homo sapiens
tgagacagagtcttgtgctgt

SEQ ID NO: 1168 - DNA, 28 bp, Homo sapiens
gcccaaatcccagcactcaccacacggc

SEQ ID NO: 1169 - DNA, 26 bp, Homo sapiens
gctcctgctcatcatctccccagtgg

SEQ ID NO: 1170 - DNA, 23 bp, Homo sapiens
atcgtccacagcccctgggtcgg

SEQ ID NO: 1171 - DNA, 24 bp, Homo sapiens
gctgcctggtgctgaactggtggg

SEQ ID NO: 1172 - DNA, 24 bp, Homo sapiens
ggctctgcccagcccctacccacg

SEQ ID NO: 1173 - DNA, 27 bp, Homo sapiens
gctcctggctgtcttgctgctgatagc

SEQ ID NO: 1174 - DNA, 26 bp, Homo sapiens
actctgttgcccaggctggagtaaag

SEQ ID NO: 1175 - DNA, 28 bp, Homo sapiens
gcaggcataaccagggatttctgaggac

SEQ ID NO: 1176 - DNA, 21 bp, Homo sapiens
caaaatgattaaggacattaa

SEQ ID NO: 1177 - DNA, 27 bp, Homo sapiens
ctcccacccatacctcttctgctggac

SEQ ID NO: 1178 - DNA, 25 bp, Homo sapiens
gcaagaaggacctcaccgcaactat

SEQ ID NO: 1179 - DNA, 27 bp, Homo sapiens
gcctcgcagcccttccaagcctcagaa

SEQ ID NO: 1180 - DNA, 24 bp, Homo sapiens
cccgccgtctgcccactcctctag

SEQ ID NO: 1181 - DNA, 24 bp, Homo sapiens
aagatcttggctcgctacaacctc

SEQ ID NO: 1182 - DNA, 28 bp, Homo sapiens
cctctctacaagacaataggcagcatct

SEQ ID NO: 1183 - DNA, 21 bp, Homo sapiens
gttttcctgagtaatagttac

SEQ ID NO: 1184 - DNA, 25 bp, Homo sapiens
gtattgatgtgtgatgaataaattg

SEQ ID NO: 1185 - DNA, 24 bp, Homo sapiens
tttaccgattagatacctgatcat

SEQ ID NO: 1186 - DNA, 22 bp, Homo sapiens
gaagcaggggcggggcgagagg

SEQ ID NO: 1187 - DNA, 23 bp, Homo sapiens
tagcatgcacaggttccagggat

SEQ ID NO: 1188 - DNA, 28 bp, Homo sapiens
tgccactatcaacctgacaccaaagcca

SEQ ID NO: 1189 - DNA, 26 bp, Homo sapiens
gcgctcctgccctggggcctcgtctt

SEQ ID NO: 1190 - DNA, 28 bp, Homo sapiens
ccgctaaagagaggaaaagtgaactgga

SEQ ID NO: 1191 - DNA, 23 bp, Homo sapiens
ttaacaatattctttcttgaaat

SEQ ID NO: 1192 - DNA, 28 bp, Homo sapiens
cgatacatttccagatagttttgtggca

SEQ ID NO: 1193 - DNA, 24 bp, Homo sapiens
gagtgagaccctgtcccagaaaaa

SEQ ID NO: 1194 - DNA, 28 bp, Homo sapiens
caagcggagcctggagcatctcgtgagg

SEQ ID NO: 1195 - DNA, 28 bp, Homo sapiens
cccaggctctctccatcgcaccttctgc

SEQ ID NO: 1196 - DNA, 21 bp, Homo sapiens
gggggcaggccaggggacagg

SEQ ID NO: 1197 - DNA, 28 bp, Homo sapiens
gaaactggtccctgccaaactggaaacg

SEQ ID NO: 1198 - DNA, 26 bp, Homo sapiens
ctggatatttatacaagagactggat

SEQ ID NO: 1199 - DNA, 28 bp, Homo sapiens
ggcacaggaggaacataccaaaacatac

SEQ ID NO: 1200 - DNA, 26 bp, Homo sapiens
tcgtgtctcactgccgcttccacctc

SEQ ID NO: 1201 - DNA, 24 bp, Homo sapiens
cgctcccctcggctacgctgccac

SEQ ID NO: 1202 - DNA, 26 bp, Homo sapiens
gggtgcccagtctgccacaacctgtc

SEQ ID NO: 1203 - DNA, 22 bp, Homo sapiens
gtggctcacacctgtaatccca

SEQ ID NO: 1204 - DNA, 38 bp, Homo sapiens
agtacatatcgagaatgggcatctctacccttgttagg

SEQ ID NO: 1205 - DNA, 39 bp, Homo sapiens
atttattcgagccgcttgagcatagagcttatggagatg

SEQ ID NO: 1206 - DNA, 32 bp, Homo sapiens
atagctccagatcgagtctgaggagtgagcgg

SEQ ID NO: 1207 - DNA, 40 bp, Homo sapiens
tctctctctttttttttttcgagatccttaggggaccttg

SEQ ID NO: 1208 - DNA, 36 bp, Homo sapiens
tcaaacagtcgaacaattcttctgcctcagcctccc

SEQ ID NO: 1209 - DNA, 37 bp, Homo sapiens
tggcttcttcttaactttctcgggtgagtcgaagagc

SEQ ID NO: 1210 - DNA, 40 bp, Homo sapiens
agaggggcccagggaacctcgaatcttctgagggagcgtg

SEQ ID NO: 1211 - DNA, 33 bp, Homo sapiens
accttcgatacttcccctgggtgactctgacag

SEQ ID NO: 1212 - DNA, 40 bp, Homo sapiens
agagaatcacctgagccttcgagttgcaaatttctgtttt

SEQ ID NO: 1213 - DNA, 28 bp, Homo sapiens
aaaaggcctcgacccttgcacgggcgca

SEQ ID NO: 1214 - DNA, 40 bp, Homo sapiens
tggggcaggcttggcctgtcgacaccaatgtcttggatgt

SEQ ID NO: 1215 - DNA, 40 bp, Homo sapiens
gatgggttatccaactcctcgaaagctttcttttctaagc

SEQ ID NO: 1216 - DNA, 40 bp, Homo sapiens
atgttggccaggatggtctcgagatcagcctggtcaacat

SEQ ID NO: 1217 - DNA, 35 bp, Homo sapiens
tgccaggcacgaacatggtcactcgaaattacgtg

SEQ ID NO: 1218 - DNA, 35 bp, Homo sapiens
acaccaccgttgggatgaaagatggcctcgataca

SEQ ID NO: 1219 - DNA, 32 bp, Homo sapiens
aatggcactcgagctctacagccagtacctgg

SEQ ID NO: 1220 - DNA, 30 bp, Homo sapiens
actagtcccgcttgcttcgatgcctgggct

SEQ ID NO: 1221 - DNA, 40 bp, Homo sapiens
ccgggctcagcgtccttgtcgaaaccgaaagcaaagagga

SEQ ID NO: 1222 - DNA, 34 bp, Homo sapiens
agcacaatcgatacttcccctgggtgactctgac

SEQ ID NO: 1223 - DNA, 40 bp, Homo sapiens
atgttggccaggctggtctcgagaccagcctgggccaaca

SEQ ID NO: 1224 - DNA, 37 bp, Homo sapiens
tcctctgcccatcttcgatggttttccatgacgattg

SEQ ID NO: 1225 - DNA, 40 bp, Homo sapiens
acagccccagcctcggaatcgacctcagcttcattcttct

SEQ ID NO: 1226 - DNA, 31 bp, Homo sapiens
agcagcctctgctctgcccacctcgaagctt

SEQ ID NO: 1227 - DNA, 34 bp, Homo sapiens
aggccatgttttggagacagtgatcgagcatttc

SEQ ID NO: 1228 - DNA, 32 bp, Homo sapiens
tttgctgtgagtagaggatgtcgagggctggg

SEQ ID NO: 1229 - DNA, 40 bp, Homo sapiens
accaccctgccaagtacctcgactcgggttcttccctgca

SEQ ID NO: 1230 - DNA, 40 bp, Homo sapiens
atgttgaccaggctggtctcgagaccagcctggcaaacat

SEQ ID NO: 1231 - DNA, 34 bp, Homo sapiens
tctagtcgatacttcccctgggtgactctgacag

SEQ ID NO: 1232 - DNA, 40 bp, Homo sapiens
tagctttgaggatttcgttcgaattttaagctccacaaat

SEQ ID NO: 1233 - DNA, 40 bp, Homo sapiens
ccacgaggttccccggcctcgaggtgtcagatatttttct

SEQ ID NO: 1234 - DNA, 40 bp, Homo sapiens
ttatgtgcaattgtaacttcgatgccagagagggagctcg

SEQ ID NO: 1235 - DNA, 40 bp, Homo sapiens
gaatgggtggagtccaagtcgactccaaaaccctaaaaca

SEQ ID NO: 1236 - DNA, 40 bp, Homo sapiens
atgttttggagacagtgatcgaagtttgtgctctttgtga

SEQ ID NO: 1237 - DNA, 32 bp, Homo sapiens
aggcctgggaatagggaaacggttcgatttgg

SEQ ID NO: 1238 - DNA, 40 bp, Homo sapiens
ttggctgctcttttacactcgagccagcgaaaagacagaa

SEQ ID NO: 1239 - DNA, 33 bp, Homo sapiens
tcgtgtctcgattttccagcagtgtgcctggag

SEQ ID NO: 1240 - DNA, 40 bp, Homo sapiens
aacggttggatgagggactcgagtatagcattcaattgct

SEQ ID NO: 1241 - DNA, 37 bp, Homo sapiens
agaatcaaattctcgaccagcttggccccatctaaag

SEQ ID NO: 1242 - DNA, 40 bp, Homo sapiens
agaagagatattccttcatcgactggtcacgatgggctgc

SEQ ID NO: 1243 - DNA, 36 bp, Homo sapiens
aggccatgttttggagacagtgatcgacagatctcc

SEQ ID NO: 1244 - DNA, 32 bp, Homo sapiens
accgttgggatgaaagatggcctcgagctggt

SEQ ID NO: 1245 - DNA, 40 bp, Homo sapiens
aggttggccatgctgggttcgagagcagcctgggcaacac

SEQ ID NO: 1246 - DNA, 40 bp, Homo sapiens
ttcttgctttcttgctttctttcttgctttcgataacctg

SEQ ID NO: 1247 - DNA, 40 bp, Homo sapiens
gaaaaccatttggccttttcgaatcttctgagggagcgtg

SEQ ID NO: 1248 - DNA, 33 bp, Homo sapiens
tctctctctctctctcgaactcctgacttcggc

SEQ ID NO: 1249 - DNA, 32 bp, Homo sapiens
actgcgtgttcccagcaccctcgattccatct

SEQ ID NO: 1250 - DNA, 33 bp, Homo sapiens
aggtggcaaaactcgaaagaacagcagctcgcg

SEQ ID NO: 1251 - DNA, 32 bp, Homo sapiens
ttttgttcgacccttctgctttctctccaggg

SEQ ID NO: 1252 - DNA, 40 bp, Homo sapiens
tctagcatatgggtagagtcgacctctctgggctcaggtg

SEQ ID NO: 1253 - DNA, 36 bp, Homo sapiens
tgcccattctcgatatgtacttcttcccctctgagg

SEQ ID NO: 1254 - DNA, 39 bp, Homo sapiens
agcggctcgaataaataaataaatatgccaggcgcagtg

SEQ ID NO: 1255 - DNA, 31 bp, Homo sapiens
ttcccgctcactcctcagactcgatctggag

SEQ ID NO: 1256 - DNA, 40 bp, Homo sapiens
caaggtcccctaaggatctcgaaaaaaaaaaagagagaga

SEQ ID NO: 1257 - DNA, 36 bp, Homo sapiens
tgggaggctgaggcagaagaattgttcgactgtttg

SEQ ID NO: 1258 - DNA, 37 bp, Homo sapiens
tgggctcttcgactcacccgagaaagttaagaagaag

SEQ ID NO: 1259 - DNA, 40 bp, Homo sapiens
cacgctccctcagaagattcgaggttccctgggcccctct

SEQ ID NO: 1260 - DNA, 33 bp, Homo sapiens
ctgtcagagtcacccaggggaagtatcgaaggt

SEQ ID NO: 1261 - DNA, 40 bp, Homo sapiens
aaaacagaaatttgcaactcgaaggctcaggtgattctct

SEQ ID NO: 1262 - DNA, 32 bp, Homo sapiens
agggtcgaggccttttccccattgtcttggct

SEQ ID NO: 1263 - DNA, 40 bp, Homo sapiens
acatccaagacattggtgtcgacaggccaagcctgcccca

SEQ ID NO: 1264 - DNA, 40 bp, Homo sapiens
gcttagaaaagaaagctttcgaggagttggataacccatc

SEQ ID NO: 1265 - DNA, 40 bp, Homo sapiens
atgttgaccaggctgatctcgagaccatcctggccaacat

SEQ ID NO: 1266 - DNA, 35 bp, Homo sapiens
tcacgtaatttcgagtgaccatgttcgtgcctggc

SEQ ID NO: 1267 - DNA, 34 bp, Homo sapiens
tgtatcgaggccatctttcatcccaacggtggtg

SEQ ID NO: 1268 - DNA, 32 bp, Homo sapiens
tccaggtactggctgtagagctcgagtgccat

SEQ ID NO: 1269 - DNA, 30 bp, Homo sapiens
agcccaggcatcgaagcaagcgggactagt

SEQ ID NO: 1270 - DNA, 40 bp, Homo sapiens
tcctctttgctttcggtttcgacaaggacgctgagcccgg

SEQ ID NO: 1271 - DNA, 34 bp, Homo sapiens
gtcagagtcacccaggggaagtatcgattgtgct

SEQ ID NO: 1272 - DNA, 40 bp, Homo sapiens
tgttggcccaggctggtctcgagaccagcctggccaacat

SEQ ID NO: 1273 - DNA, 35 bp, Homo sapiens
accatcgaagatgggcagaggatatgaagtgagcc

SEQ ID NO: 1274 - DNA, 40 bp, Homo sapiens
agaagaatgaagctgaggtcgattccgaggctggggctgt

SEQ ID NO: 1275 - DNA, 29 bp, Homo sapiens
aagcttcgaggtgggcagagcagaggctg

SEQ ID NO: 1276 - DNA, 38 bp, Homo sapiens
atgctcgatcactgtctccaaaacatggccttatcacc

SEQ ID NO: 1277 - DNA, 32 bp, Homo sapiens
agccctcgacatcctctactcacagcaaaggg

SEQ ID NO: 1278 - DNA, 40 bp, Homo sapiens
tgcagggaagaacccgagtcgaggtacttggcagggtggt

SEQ ID NO: 1279 - DNA, 40 bp, Homo sapiens
atgtttgccaggctggtctcgagaccagcctggtcaacat

SEQ ID NO: 1280 - DNA, 34 bp, Homo sapiens
ctgtcagagtcacccaggggaagtatcgactaga

SEQ ID NO: 1281 - DNA, 40 bp, Homo sapiens
atttgtggagcttaaaattcgaacgaaatcctcaaagcta

SEQ ID NO: 1282 - DNA, 40 bp, Homo sapiens
agaaaaatatctgacacctcgaggccggggaacctcgtgg

SEQ ID NO: 1283 - DNA, 40 bp, Homo sapiens
cgagctccctctctggcatcgaagttacaattgcacataa

SEQ ID NO: 1284 - DNA, 40 bp, Homo sapiens
tgttttagggttttggagtcgacttggactccacccattc

SEQ ID NO: 1285 - DNA, 40 bp, Homo sapiens
tcacaaagagcacaaacttcgatcactgtctccaaaacat

SEQ ID NO: 1286 - DNA, 35 bp, Homo sapiens
accaaatcgaaccgtttccctattcccaggcctag

SEQ ID NO: 1287 - DNA, 40 bp, Homo sapiens
ttctgtcttttcgctggctcgagtgtaaaagagcagccaa

SEQ ID NO: 1288 - DNA, 34 bp, Homo sapiens
tccaggcacactgctggaaaatcgagacacgatc

SEQ ID NO: 1289 - DNA, 40 bp, Homo sapiens
agcaattgaatgctatactcgagtccctcatccaaccgtt

SEQ ID NO: 1290 - DNA, 37 bp, Homo sapiens
ctttagatggggccaagctggtcgagaatttgattct

SEQ ID NO: 1291 - DNA, 40 bp, Homo sapiens
gcagcccatcgtgaccagtcgatgaaggaatatctcttct

SEQ ID NO: 1292 - DNA, 38 bp, Homo sapiens
tctgtcgatcactgtctccaaaacatggccttatcacc

SEQ ID NO: 1293 - DNA, 32 bp, Homo sapiens
accagctcgaggccatctttcatcccaacggt

SEQ ID NO: 1294 - DNA, 40 bp, Homo sapiens
gtgttgcccaggctgctctcgaacccagcatggccaacct

SEQ ID NO: 1295 - DNA, 40 bp, Homo sapiens
aggttatcgaaagcaagaaagaaagcaagaaagcaagaag

SEQ ID NO: 1296 - DNA, 40 bp, Homo sapiens
cacgctccctcagaagattcgaaaaggccaaatggttttc

SEQ ID NO: 1297 - DNA, 33 bp, Homo sapiens
agccgaagtcaggagttcgagagagagagagag

SEQ ID NO: 1298 - DNA, 32 bp, Homo sapiens
agatggaatcgagggtgctgggaacacgcagt

SEQ ID NO: 1299 - DNA, 33 bp, Homo sapiens
tcgcgagctgctgttctttcgagttttgccacc

SEQ ID NO: 1300 - DNA, 34 bp, Homo sapiens
tcccctggagagaaagcagaagggtcgaacaaaa

SEQ ID NO: 1301 - DNA, 40 bp, Homo sapiens
cacctgagcccagagaggtcgactctacccatatgctaga

SEQ ID NO: 1302 - DNA, 60 bp, Homo sapiens; identical to SEQ ID NO: 26

SEQ ID NO: 1303 - DNA, 60 bp, Homo sapiens
tggactgggctaatgtggaaagtgaggatcgagaatgaagaaggaacagtcacgaaggga

SEQ ID NO: 1304 - DNA, 60 bp, Homo sapiens
tatttatttttatttttatttatttttttcgattgtgttcaccaggacggcaggctatta

SEQ ID NO: 1305 - DNA, 60 bp, Homo sapiens
cacatggctaaaaggaagactgacaatttcgagcttggtgtggtcaagggccaggaggag

SEQ ID NO: 1306 - DNA, 60 bp, Homo sapiens
taatattggctttggctaatcagagatttcgacaggaggatggtggggaggggaggccag

SEQ ID NO: 1307 - DNA, 60 bp, Homo sapiens
ggtgggtagatcacttgaggtcaggagttcgaactcctgacctcaagtgatccacgccca

SEQ ID NO: 1308 - DNA, 60 bp, Homo sapiens
ggaggcagaagttgtgggcatgctgaactcgaggaaatccattgacatgaaaaacgtcaa

SEQ ID NO: 1309 - DNA, 60 bp, Homo sapiens
ttcctgcaggtgatggttcatgacaatatcgactgagctgccaagtatttcatacatggg

SEQ ID NO: 1310 - DNA, 60 bp, Homo sapiens
gagtgtgatgaagaaaagtaaggcaggatcgatgaattctagggcatgtaaatgctcaag

SEQ ID NO: 1311 - DNA, 60 bp, Homo sapiens
gcgaatgagctccctgtgtcacacagcatcgagagtcctgagcaccatcccaatccctgt

SEQ ID NO: 1312 - DNA, 60 bp, Homo sapiens
tctaggcttcaagagagagaatccaatgtcgatttagccttccataactatcctcagaca

SEQ ID NO: 1313 - DNA, 60 bp, Homo sapiens
aatggtgtctatagccaggtttttgtaatcgaaggaccccttgctgggggcgcctaaaga

SEQ ID NO: 1314 - DNA, 60 bp, Homo sapiens
gagtgtgatgaagaaaagtaaggcaggatcgaatatttgcacatttctcgttgacccttc

SEQ ID NO: 1315 - DNA, 60 bp, Homo sapiens
ctatatttttcactcacatccctagttatcgagaacatcctggctaatgtgatgaaaccc

SEQ ID NO: 1316 - DNA, 60 bp, Homo sapiens
acgctgaactaattgggctgaagtatggtcgaacaggttacgagcatcagctgttacatg

SEQ ID NO: 1317 - DNA, 60 bp, Homo sapiens
cattggtcacaatgggtcacgtgcctattcgagaccacccagcaaagccccaaggacaca

SEQ ID NO: 1318 - DNA, 60 bp, Homo sapiens
tgcacctctcttttgaatccctcccccatcgaggttaatccctctccacagggcggcacc

SEQ ID NO: 1319 - DNA, 60 bp, Homo sapiens
agagagagtaacaaatacgaggagggtttcgatgttactgattctgtccccatcttcgcg

SEQ ID NO: 1320 - DNA, 60 bp, Homo sapiens
actctcttacccaagtttcctaacaggatcgacacccagggtcattcacacaacacggct

SEQ ID NO: 1321 - DNA, 60 bp, Homo sapiens
ctgaagccaacacacgcaaaggcaaacgtcgaaactgatcatttaggttttccttttaag

SEQ ID NO: 1322 - DNA, 60 bp, Homo sapiens
gtttacagaccttggatgcaggctcttctcgaggggaaaaaggagcaagttgggaggtga

SEQ ID NO: 1323 - DNA, 60 bp, Homo sapiens
cctgtggcctgataaggtattgaccccatcgagattttattaattagtggcagtcataat

SEQ ID NO: 1324 - DNA, 60 bp, Homo sapiens
aggggtggacacctgctcaacagagaattcgaccccatcctctggccctcagttctgtcc

SEQ ID NO: 1325 - DNA, 60 bp, Homo sapiens
agcctcttccaacactgaggatcacaactcgatgatataatactctgctgactacatttt

SEQ ID NO: 1326 - DNA, 60 bp, Homo sapiens
ggagctaagatgactaatttgatgattttcgaactcctgacctcgtgatcttcccacctt

SEQ ID NO: 1327 - DNA, 60 bp, Homo sapiens
tcaaatgagcatccccattctgtttacatcgagaccagcctggccaaaatggtgaaaacc

SEQ ID NO: 1328 - DNA, 60 bp, Homo sapiens
tgccagcttttcttgccattctggccattcgacaatattgttttattttagcatcaattt

SEQ ID NO: 1329 - DNA, 60 bp, Homo sapiens
tgactcacgagggaagtggttgttgacctcgattcctctcctctcagtgacaaccaaaga

SEQ ID NO: 1330 - DNA, 60 bp, Homo sapiens
tttttaaaaaccctatacaaatgctgattcgactgcatcagcctctacgggaatctcctc

SEQ ID NO: 1331 - DNA, 60 bp, Homo sapiens
acattccacttaactgcaacttggcttctcgaatgtggtagccacagactgcagtcagga

SEQ ID NO: 1332 - DNA, 60 bp, Homo sapiens
aacttgacaacctgtaagggattctgaatcgacaggggagaaagtgccggcccagagaaa

SEQ ID NO: 1333 - DNA, 60 bp, Homo sapiens
catatacagccagtccctgaggaaggagtcgatgaaggaagaaaagagacgtggttttct

SEQ ID NO: 1334 - DNA, 60 bp, Homo sapiens
gcacgatatttttgcaagagtagacaaatcgaagacccgagccggcgcttgcgcacttag

SEQ ID NO: 1335 - DNA, 60 bp, Homo sapiens
cctggctccttcctgtattatgctcccttcgagtattcttctgggtgtttctatgaggat

SEQ ID NO: 1336 - DNA, 60 bp, Homo sapiens
caaatattaaataactgctaaaagtgcttcgaactcctgagctcaggtgatccacccacc

SEQ ID NO: 1337 - DNA, 60 bp, Homo sapiens
tcagtctcacaagccatatttcaagtgctcgagaaccaaatagcctaagtcaataaaata

SEQ ID NO: 1338 - DNA, 60 bp, Homo sapiens
aacagttgtcatctatgtaatggacacatcgaactcctgaccttgtgatcagcccgtctc

SEQ ID NO: 1339 - DNA, 60 bp, Homo sapiens
caccaggcccaggttctcgcagaaaggctcgaacttgctagacaaagactttgaatcagt

SEQ ID NO: 1340 - DNA, 60 bp, Homo sapiens
ttcatagtgcctggaatgtagtaggatctcgatttgtataatttcaattaaaaccatcca

SEQ ID NO: 1341 - DNA, 60 bp, Homo sapiens
ctgcctatctttggctctaacccacagatcgagagatcaggaaatatttagctcaatctt

SEQ ID NO: 1342 - DNA, 60 bp, Homo sapiens
agccagaggtcgtaaaacgatgatagaatcgaactcctggtctcaagtgatccaccagcc

SEQ ID NO: 1343 - DNA, 60 bp, Homo sapiens
cacggaggtcttttaagcttagttggaatcgacctttcaggctcaagcaatcctcccacc

SEQ ID NO: 1344 - DNA, 60 bp, Homo sapiens; identical to SEQ ID NO: 26

SEQ ID NO: 1345 - DNA, 60 bp, Homo sapiens
tgggcaaagaaagataaggaaggagatttcgatttctgggcttggccattatcaccctgg

SEQ ID NO: 1346 - DNA, 60 bp, Homo sapiens
ctgataaaaggtaacgttatattgtttgtcgagaccatgctggctaatatgatgaaaccc

SEQ ID NO: 1347 - DNA, 60 bp, Homo sapiens
gggttttgccatgttggccaggctggtctcgagaccagcctggccaacatggcgaaaccc

SEQ ID NO: 1348 - DNA, 60 bp, Homo sapiens
ggtgctaggaagcaaggatactgcattgtcgactgcatttctgccttcgggggcttgacc

SEQ ID NO: 1349 - DNA, 60 bp, Homo sapiens
agggttcaacttcagaaagagggggctctcgaagatccccgggcagcgcgaaaggaggtt

SEQ ID NO: 1350 - DNA, 60 bp, Homo sapiens
ggggccacatccaatgtcttcgtgatgttcgaagcacaaagctcacttactcccggaacg

SEQ ID NO: 1351 - DNA, 60 bp, Homo sapiens
agtttgcataagagaagaaattaatttttcgacctcccaaagtgctaggattacaagcgt

SEQ ID NO: 1352 - DNA, 22 bp, Homo sapiens
tgcatgagatgaaccagggtgg

SEQ ID NO: 1353 - DNA, 22 bp, Homo sapiens
aggtatttcttctttttctatt

SEQ ID NO: 1354 - DNA, 26 bp, Homo sapiens
ctccttgttcagactcagggaataag

SEQ ID NO: 1355 - DNA, 26 bp, Homo sapiens
ggctccaggtctcttctgtctcttga

SEQ ID NO: 1356 - DNA, 26 bp, Homo sapiens
gtgggaaggaagggtgtcatctatac

SEQ ID NO: 1357 - DNA, 26 bp, Homo sapiens
cataaagcctctctccaatcgctttc

SEQ ID NO: 1358 - DNA, 26 bp, Homo sapiens
agcagattagaaccacagtgagttgc

SEQ ID NO: 1359 - DNA, 26 bp, Homo sapiens
ggaggacacctgtttgctgagttggt

SEQ ID NO: 1360 - DNA, 22 bp, Homo sapiens
acagatacaaataagcttgacc

SEQ ID NO: 1361 - DNA, 22 bp, Homo sapiens
tgtacagtcccagctactcagg

SEQ ID NO: 1362 - DNA, 21 bp, Homo sapiens
actgcagcactattcacaaca

SEQ ID NO: 1363 - DNA, 21 bp, Homo sapiens
atccttttaatgttgagacca

SEQ ID NO: 1364 - DNA, 23 bp, Homo sapiens
gatgcttcccaaggctacctggt

SEQ ID NO: 1365 - DNA, 26 bp, Homo sapiens
cagaaaatcagaaagcctccaccaaa

SEQ ID NO: 1366 - DNA, 26 bp, Homo sapiens
ctgcgaaagaacctcaaaagttagcc

SEQ ID NO: 1367 - DNA, 26 bp, Homo sapiens
tgggataatcttactcattgcctggc

SEQ ID NO: 1368 - DNA, 26 bp, Homo sapiens
ttgttattccaggtggcaatgctgag

SEQ ID NO: 1369 - DNA, 26 bp, Homo sapiens
ggcaggacttacccaacatcaggact

SEQ ID NO: 1370 - DNA, 26 bp, Homo sapiens
gcttgttttgaatgaggttaggcagc

SEQ ID NO: 1371 - DNA, 25 bp, Homo sapiens
gccatcacaggttaggagacagacc

SEQ ID NO: 1372 - DNA, 26 bp, Homo sapiens
caccccttccccttgcattggtttcc

SEQ ID NO: 1373 - DNA, 26 bp, Homo sapiens
gctgaccgttctttccttcattcatg

SEQ ID NO: 1374 - DNA, 26 bp, Homo sapiens
ccaggaacaaaacaaaccccaggtaa

SEQ ID NO: 1375 - DNA, 26 bp, Homo sapiens
aagaagccaggcaagaagacacgagc

SEQ ID NO: 1376 - DNA, 24 bp, Homo sapiens
acacttattaagcacctatatgtg

SEQ ID NO: 1377 - DNA, 24 bp, Homo sapiens
ttcagaaattatcatcattctaca

SEQ ID NO: 1378 - DNA, 26 bp, Homo sapiens
ccagcctgggcaacagagtgagactg

SEQ ID NO: 1379 - DNA, 26 bp, Homo sapiens
ctgtcactcgggctacagtgcagtgg

SEQ ID NO: 1380 - DNA, 23 bp, Homo sapiens
gagccagccagtcctcatcaagg

SEQ ID NO: 1381 - DNA, 23 bp, Homo sapiens
gcccttagcccttctgtatgtgc

SEQ ID NO: 1382 - DNA, 23 bp, Homo sapiens
ggtgccacagtgccttacatcac

SEQ ID NO: 1383 - DNA, 23 bp, Homo sapiens
tttctgggtcactcccgcctgtg

SEQ ID NO: 1384 - DNA, 24 bp, Homo sapiens
gcctggtgaactctgtcgtgtggg

SEQ ID NO: 1385 - DNA, 24 bp, Homo sapiens
gtgattcataaagcagctcttttc

SEQ ID NO: 1386 - DNA, 23 bp, Homo sapiens
cgggctgtctcctgaatggtgtg

SEQ ID NO: 1387 - DNA, 23 bp, Homo sapiens
tgtgccaggtgttgtgctgatgc

SEQ ID NO: 1388 - DNA, 25 bp, Homo sapiens
gcaggatcagtgccagggcaggggg

SEQ ID NO: 1389 - DNA, 25 bp, Homo sapiens
ggaggaaggagcaggcgcgagcgct

SEQ ID NO: 1390 - DNA, 25 bp, Homo sapiens
ccagggcgctgtggttttgcgtggc

SEQ ID NO: 1391 - DNA, 25 bp, Homo sapiens
aagaaaagtggatgagctgaaatga

SEQ ID NO: 1392 - DNA, 24 bp, Homo sapiens
gagggcttctgtgagattgggcac

SEQ ID NO: 1393 - DNA, 23 bp, Homo sapiens
ggcagagaggcaatgtcaggaag

SEQ ID NO: 1394 - DNA, 26 bp, Homo sapiens
gaaaagagtcaacacggagcagaaag

SEQ ID NO: 1395 - DNA, 26 bp, Homo sapiens
gcaccacccagagcaccaaccttatt

SEQ ID NO: 1396 - DNA, 21 bp, Homo sapiens
accgccgcaccctccgggccg

SEQ ID NO: 1397 - DNA, 21 bp, Homo sapiens
tcccacccatccctccatcca

SEQ ID NO: 1398 - DNA, 26 bp, Homo sapiens
aagtgggagagtgaagggagaaggga

SEQ ID NO: 1399 - DNA, 26 bp, Homo sapiens
caagcacggttgtaggagttgtaagt

SEQ ID NO: 1400 - DNA, 23 bp, Homo sapiens
ggcgggagagcgatgcttggatt

SEQ ID NO: 1401 - DNA, 23 bp, Homo sapiens
ctctccgctgccttcttgtccta

SEQ ID NO: 1402 - DNA, 25 bp, Homo sapiens
taaaagaactactaattattatgtt

SEQ ID NO: 1403 - DNA, 25 bp, Homo sapiens
catccaggctggagtacactggcgc

SEQ ID NO: 1404 - DNA, 26 bp, Homo sapiens
ttgagcttcacaaaggttccactgaa

SEQ ID NO: 1405 - DNA, 26 bp, Homo sapiens
tttcaccgtgatttagactctactac

SEQ ID NO: 1406 - DNA, 23 bp, Homo sapiens
ggtaagcgtaggagcagagcaac

SEQ ID NO: 1407 - DNA, 23 bp, Homo sapiens
gggtgggaaaggtcctctggtca

SEQ ID NO: 1408 - DNA, 22 bp, Homo sapiens
gggctcaagtgatcctcctgcc

SEQ ID NO: 1409 - DNA, 22 bp, Homo sapiens
gaccatgcagtgaaaatgagga

SEQ ID NO: 1410 - DNA, 26 bp, Homo sapiens
ccttgtcataatgggaaactcagggc

SEQ ID NO: 1411 - DNA, 26 bp, Homo sapiens
cctgggaagcaaaagaaaggaggaca

SEQ ID NO: 1412 - DNA, 26 bp, Homo sapiens
gagttttggctgctgaagggaaggca

SEQ ID NO: 1413 - DNA, 25 bp, Homo sapiens
atggtcattcactcaaatcctgggc

SEQ ID NO: 1414 - DNA, 23 bp, Homo sapiens
ccccaagtctctctgtccttcct

SEQ ID NO: 1415 - DNA, 23 bp, Homo sapiens
catcaaagggagtggctctgccc

SEQ ID NO: 1416 - DNA, 26 bp, Homo sapiens
gatcatgacacattaattcttaaatt

SEQ ID NO: 1417 - DNA, 26 bp, Homo sapiens
gtaggaggggagcgccggcccggccg

SEQ ID NO: 1418 - DNA, 23 bp, Homo sapiens
cccaggttggaggcatctctgac

SEQ ID NO: 1419 - DNA, 23 bp, Homo sapiens
ccaggcgaatctgaaggcaggat

SEQ ID NO: 1420 - DNA, 26 bp, Homo sapiens
gctgttccagacctaaaacccaatgt

SEQ ID NO: 1421 - DNA, 26 bp, Homo sapiens
aagatgacttccttccttagcccagc

SEQ ID NO: 1422 - DNA, 26 bp, Homo sapiens
gagtctcacactgtagccacgaactg

SEQ ID NO: 1423 - DNA, 26 bp, Homo sapiens
gctctgccatacagttcattctcatc

SEQ ID NO: 1424 - DNA, 25 bp, Homo sapiens
gctctgcacaccctgcctaaagtgg

SEQ ID NO: 1425 - DNA, 25 bp, Homo sapiens
gctctttgtgggcccagacggtgtt

SEQ ID NO: 1426 - DNA, 25 bp, Homo sapiens
cataaattcctagtttcctgtgcaa

SEQ ID NO: 1427 - DNA, 25 bp, Homo sapiens
tttctaattctacttggtttctttt

SEQ ID NO: 1428 - DNA, 23 bp, Homo sapiens
ctctcccctgaagttcccacctg

SEQ ID NO: 1429 - DNA, 23 bp, Homo sapiens
gcagaagttggagcctctacctc

SEQ ID NO: 1430 - DNA, 22 bp, Homo sapiens
gataaagcttcaccatctctgc

SEQ ID NO: 1431 - DNA, 22 bp, Homo sapiens
agggaacagtttgctctcctct

SEQ ID NO: 1432 - DNA, 23 bp, Homo sapiens
gagacacgcaagtcttgcatctg

SEQ ID NO: 1433 - DNA, 23 bp, Homo sapiens
actcttgtgctcaagtgattagc

SEQ ID NO: 1434 - DNA, 22 bp, Homo sapiens
ctgatctaacccccaaaatcct

SEQ ID NO: 1435 - DNA, 22 bp, Homo sapiens
agtgctagctatttggtcttga

SEQ ID NO: 1436 - DNA, 26 bp, Homo sapiens
agctcagtgaacatgacccgatggtg

SEQ ID NO: 1437 - DNA, 26 bp, Homo sapiens
atagtgtaagctaggtaatatttctt

SEQ ID NO: 1438 - DNA, 23 bp, Homo sapiens
gaggaggtagacaggatgctgag

SEQ ID NO: 1439 - DNA, 23 bp, Homo sapiens
agcccaggacaagtgtgaccagg

SEQ ID NO: 1440 - DNA, 23 bp, Homo sapiens
accccgacattctccacctcctg

SEQ ID NO: 1441 - DNA, 23 bp, Homo sapiens
attctccttcctcagcctcccga

SEQ ID NO: 1442 - DNA, 23 bp, Homo sapiens
tccagcacccagtgtggagtgca

SEQ ID NO: 1443 - DNA, 23 bp, Homo sapiens
ctttttctttttttttgagacag

SEQ ID NO: 1444 - DNA, 26 bp, Homo sapiens
gtgtgagccatctgtttatgcctttt

SEQ ID NO: 1445 - DNA, 26 bp, Homo sapiens
gggtttgaccacagacctgagagatg

SEQ ID NO: 1446 - DNA, 24 bp, Homo sapiens
agtgtcacacagtaaaaggtatag

SEQ ID NO: 1447 - DNA, 24 bp, Homo sapiens
ggaggagaccctttgaaggtccct

SEQ ID NO: 1448 - DNA, 23 bp, Homo sapiens
caccaagaagcgaccacagcaca

SEQ ID NO: 1449 - DNA, 23 bp, Homo sapiens
gaagaacagcaccgcccagcgtt

SEQ ID NO: 1450 - DNA, 23 bp, Homo sapiens
gttccaatttttcaatgaggtaa

SEQ ID NO: 1451 - DNA, 23 bp, Homo sapiens
ggcttgagcaagcctttcaatcc

SEQ ID NO: 1452 - DNA, 21 bp, Homo sapiens
cctgcatgagatgaaccaggg

SEQ ID NO: 1453 - DNA, 28 bp, Homo sapiens
gactccttgttcagactcagggaataag

SEQ ID NO: 1454 - DNA, 22 bp, Homo sapiens
ttttgaccatttgagcaggtac

SEQ ID NO: 1455 - DNA, 28 bp, Homo sapiens
aaagcagattagaaccacagtgagttgc

SEQ ID NO: 1456 - DNA, 26 bp, Homo sapiens
ttgacaagtcttttaagaaaaagtac

SEQ ID NO: 1457 - DNA, 24 bp, Homo sapiens
caaaacatgatctcaaagagatat

SEQ ID NO: 1458 - DNA, 25 bp, Homo sapiens
gccagatgcttcccaaggctacctg

SEQ ID NO: 1459 - DNA, 28 bp, Homo sapiens
cactgcgaaagaacctcaaaagttagcc

SEQ ID NO: 1460 - DNA, 28 bp, Homo sapiens
gatgaaaatggcacctcaagcctcagca

SEQ ID NO: 1461 - DNA, 28 bp, Homo sapiens
tggcttgttttgaatgaggttaggcagc

SEQ ID NO: 1462 - DNA, 22 bp, Homo sapiens
tgctgctgtaacaaattaccac

SEQ ID NO: 1463 - DNA, 28 bp, Homo sapiens
ggaaaaggagactgaggaaacgctctgc

SEQ ID NO: 1464 - DNA, 23 bp, Homo sapiens
gcgagccaaacaagatctctgcc

SEQ ID NO: 1465 - DNA, 26 bp, Homo sapiens
aaattataaacttttacatcaccgca

SEQ ID NO: 1466 - DNA, 27 bp, Homo sapiens
cctgccccagccagaggatagaggagg

SEQ ID NO: 1467 - DNA, 28 bp, Homo sapiens
gggtgccacagtgccttacatcacatcc

SEQ ID NO: 1468 - DNA, 22 bp, Homo sapiens
caatgggaaggtggacagagga

SEQ ID NO: 1469 - DNA, 26 bp, Homo sapiens
ggacgggctgtctcctgaatggtgtg

SEQ ID NO: 1470 - DNA, 25 bp, Homo sapiens
gcaggcaggatcagtgccagggcag

SEQ ID NO: 1471 - DNA, 26 bp, Homo sapiens
gctgtggttttgcgtggcaagccaga

SEQ ID NO: 1472 - DNA, 28 bp, Homo sapiens
gtcaggtcagcacatcccattaggtgga

SEQ ID NO: 1473 - DNA, 28 bp, Homo sapiens
gggaaaagagtcaacacggagcagaaag

SEQ ID NO: 1474 - DNA, 23 bp, Homo sapiens
ctgcctgtgtccccccgcaccct

SEQ ID NO: 1475 - DNA, 28 bp, Homo sapiens
ggagagtgaagggagaagggacacaccc

SEQ ID NO: 1476 - DNA, 28 bp, Homo sapiens
gggtagggcgggagagcgatgcttggat

SEQ ID NO: 1477 - DNA, 23 bp, Homo sapiens
taattattatgttaaaatctggg

SEQ ID NO: 1478 - DNA, 24 bp, Homo sapiens
acctttcagaacacctttgtgtgc

SEQ ID NO: 1479 - DNA, 28 bp, Homo sapiens
ggtgaaagggtaagcgtaggagcagagc

SEQ ID NO: 1480 - DNA, 23 bp, Homo sapiens
ggaaatcctgggctcaagtgatc

SEQ ID NO: 1481 - DNA, 25 bp, Homo sapiens
gctgggcatccctgaggcttggagc

SEQ ID NO: 1482 - DNA, 28 bp, Homo sapiens
caagagttttggctgctgaagggaaggc

SEQ ID NO: 1483 - DNA, 27 bp, Homo sapiens
ctcaccgcctcacctgccttggtctgg

SEQ ID NO: 1484 - DNA, 25 bp, Homo sapiens
gttttgcctgtgtgtagatcatgac

SEQ ID NO: 1485 - DNA, 27 bp, Homo sapiens
cccacccaggttggaggcatctctgac

SEQ ID NO: 1486 - DNA, 28 bp, Homo sapiens
ggatacacaccgtgctctttcattactg

SEQ ID NO: 1487 - DNA, 28 bp, Homo sapiens
ggcaggaggattggtttggtttgacccc

SEQ ID NO: 1488 - DNA, 24 bp, Homo sapiens
aagagtttgaggacagatcgtgag

SEQ ID NO: 1489 - DNA, 21 bp, Homo sapiens
cctgtgcaatctcagtatttt

SEQ ID NO: 1490 - DNA, 28 bp, Homo sapiens
acccctctcccactccactgcctctcag

SEQ ID NO: 1491 - DNA, 22 bp, Homo sapiens
atcccatgacttcccggtagtg

SEQ ID NO: 1492 - DNA, 25 bp, Homo sapiens
ctttcccctccccctccccctcccc

SEQ ID NO: 1493 - DNA, 24 bp, Homo sapiens
acttgtatgaatgatatgttactg

SEQ ID NO: 1494 - DNA, 23 bp, Homo sapiens
agggtggctctgggcctgaggct

SEQ ID NO: 1495 - DNA, 28 bp, Homo sapiens
gggagataggaggaggtagacaggatgc

SEQ ID NO: 1496 - DNA, 28 bp, Homo sapiens
gattaccccgacattctccacctcctgg

SEQ ID NO: 1497 - DNA, 23 bp, Homo sapiens
cctctttcctcctgggttcaagc

SEQ ID NO: 1498 - DNA, 28 bp, Homo sapiens
tagtgtgagccatctgtttatgcctttt

SEQ ID NO: 1499 - DNA, 24 bp, Homo sapiens
aagtggcatctggcattccccaat

SEQ ID NO: 1500 - DNA, 24 bp, Homo sapiens
ctctgctaccttccccaccctgcg

SEQ ID NO: 1501 - DNA, 24 bp, Homo sapiens
atttttcaatgaggtaaaatgaaa

SEQ ID NO: 1502 - DNA, 21 bp, Homo sapiens
caataaagcgaaaggtatttc

SEQ ID NO: 1503 - DNA, 40 bp, Homo sapiens
tcctgtattatgctcccttcgagaagtagaatatctgaat

SEQ ID NO: 1504 - DNA, 23 bp, Homo sapiens
cctgccaagcctgccgccactgt

SEQ ID NO: 1505 - DNA, 37 bp, Homo sapiens
tggactgggctaatgtggaaagtgaggatcgagaatg

SEQ ID NO: 1506 - DNA, 22 bp, Homo sapiens
taaagcctctctccaatcgctt

SEQ ID NO: 1507 - DNA, 40 bp, Homo sapiens
tatttttatttatttttttcgattgtgttcaccaggacgg

SEQ ID NO: 1508 - DNA, 28 bp, Homo sapiens
gtaggaggacacctgtttgctgagttgg

SEQ ID NO: 1509 - DNA, 33 bp, Homo sapiens
acaatttcgagcttggtgtggtcaagggccagg

SEQ ID NO: 1510 - DNA, 26 bp, Homo sapiens
caggtgcagtggcacaggcctgtaca

SEQ ID NO: 1511 - DNA, 40 bp, Homo sapiens
tttggctaatcagagatttcgacaggaggatggtggggag

SEQ ID NO: 1512 - DNA, 24 bp, Homo sapiens
ttttccagaggctcagggcagaga

SEQ ID NO: 1513 - DNA, 40 bp, Homo sapiens
tcacttgaggtcaggagttcgaactcctgacctcaagtga

SEQ ID NO: 1514 - DNA, 28 bp, Homo sapiens
ctgtttctgagtgcggatgacattatct

SEQ ID NO: 1515 - DNA, 34 bp, Homo sapiens
aggcagaagttgtgggcatgctgaactcgaggaa

SEQ ID NO: 1516 - DNA, 28 bp, Homo sapiens
gctgggataatcttactcattgcctggc

SEQ ID NO: 1517 - DNA, 37 bp, Homo sapiens
tgcaggtgatggttcatgacaatatcgactgagctgc

SEQ ID NO: 1518 - DNA, 28 bp, Homo sapiens
ggcaggacttacccaacatcaggacttc

SEQ ID NO: 1519 - DNA, 37 bp, Homo sapiens
aggcaggatcgatgaattctagggcatgtaaatgctc

SEQ ID NO: 1520 - DNA, 25 bp, Homo sapiens
gccatcacaggttaggagacagacc

SEQ ID NO: 1521 - DNA, 33 bp, Homo sapiens
atgagctccctgtgtcacacagcatcgagagtc

SEQ ID NO: 1522 - DNA, 22 bp, Homo sapiens
tttgttcagtcaacacatatgt

SEQ ID NO: 1523 - DNA, 40 bp, Homo sapiens
aagagagagaatccaatgtcgatttagccttccataacta

SEQ ID NO: 1524 - DNA, 28 bp, Homo sapiens
gcaagaagccaggcaagaagacacgagc

SEQ ID NO: 1525 - DNA, 28 bp, Homo sapiens
ttttgtaatcgaaggaccccttgctggg

SEQ ID NO: 1526 - DNA, 23 bp, Homo sapiens
ggaattcttccccctgcctttct

SEQ ID NO: 1527 - DNA, 40 bp, Homo sapiens
aagaaaagtaaggcaggatcgaatatttgcacatttctcg

SEQ ID NO: 1528 - DNA, 26 bp, Homo sapiens
tctgtcactcgggctacagtgcagtg

SEQ ID NO: 1529 - DNA, 40 bp, Homo sapiens
cactcacatccctagttatcgagaacatcctggctaatgt

SEQ ID NO: 1530 - DNA, 28 bp, Homo sapiens
ctctgtcaggaaacaaagcccttagccc

SEQ ID NO: 1531 - DNA, 35 bp, Homo sapiens
tgggctgaagtatggtcgaacaggttacgagcatc

SEQ ID NO: 1532 - DNA, 28 bp, Homo sapiens
tatttcccaggctccaggctcctcgccg

SEQ ID NO: 1533 - DNA, 32 bp, Homo sapiens
acaatgggtcacgtgcctattcgagaccaccc

SEQ ID NO: 1534 - DNA, 22 bp, Homo sapiens
tgtgattcataaagcagctctt

SEQ ID NO: 1535 - DNA, 40 bp, Homo sapiens
ttttgaatccctcccccatcgaggttaatccctctccaca

SEQ ID NO: 1536 - DNA, 28 bp, Homo sapiens
gactatgtgccaggtgttgtgctgatgc

SEQ ID NO: 1537 - DNA, 35 bp, Homo sapiens
acgaggagggtttcgatgttactgattctgtcccc

SEQ ID NO: 1538 - DNA, 25 bp, Homo sapiens
aatgaccctgggtgtcatggaggaa

SEQ ID NO: 1539 - DNA, 40 bp, Homo sapiens
ccaagtttcctaacaggatcgacacccagggtcattcaca

SEQ ID NO: 1540 - DNA, 26 bp, Homo sapiens
caagaaaagtggatgagctgaaatga

SEQ ID NO: 1541 - DNA, 40 bp, Homo sapiens
cacacgcaaaggcaaacgtcgaaactgatcatttaggttt

SEQ ID NO: 1542 - DNA, 28 bp, Homo sapiens
aaaggcagagaggcaatgtcaggaagac

SEQ ID NO: 1543 - DNA, 32 bp, Homo sapiens
ttcccctcgagaagagcctgcatccaaggtct

SEQ ID NO: 1544 - DNA, 27 bp, Homo sapiens
ctttaggcaccacccagagcaccaacc

SEQ ID NO: 1545 - DNA, 37 bp, Homo sapiens
tcctgtggcctgataaggtattgaccccatcgagatt

SEQ ID NO: 1546 - DNA, 23 bp, Homo sapiens
catcacccatagtgtccgcaggc

SEQ ID NO: 1547 - DNA, 40 bp, Homo sapiens
acctgctcaacagagaattcgaccccatcctctggccctc

SEQ ID NO: 1548 - DNA, 28 bp, Homo sapiens
gtgtgaatcaggagaggacaaaagaggg

SEQ ID NO: 1549 - DNA, 36 bp, Homo sapiens
agcctcttccaacactgaggatcacaactcgatgat

SEQ ID NO: 1550 - DNA, 24 bp, Homo sapiens
cccctctccgctgccttcttgtcc

SEQ ID NO: 1551 - DNA, 36 bp, Homo sapiens
tgattttcgaactcctgacctcgtgatcttcccacc

SEQ ID NO: 1552 - DNA, 23 bp, Homo sapiens
ctcccgggttcaaatgatccttc

SEQ ID NO: 1553 - DNA, 40 bp, Homo sapiens
atccccattctgtttacatcgagaccagcctggccaaaat

SEQ ID NO: 1554 - DNA, 24 bp, Homo sapiens
gtttcaccgtgatttagactctac

SEQ ID NO: 1555 - DNA, 40 bp, Homo sapiens
tcttgccattctggccattcgacaatattgttttatttta

SEQ ID NO: 1556 - DNA, 28 bp, Homo sapiens
gctctgggactgcctgaccttagcaaag

SEQ ID NO: 1557 - DNA, 34 bp, Homo sapiens
actcacgagggaagtggttgttgacctcgattcc

SEQ ID NO: 1558 - DNA, 23 bp, Homo sapiens
ttctgtggctggtgggctcaaag

SEQ ID NO: 1559 - DNA, 40 bp, Homo sapiens
ccctatacaaatgctgattcgactgcatcagcctctacgg

SEQ ID NO: 1560 - DNA, 23 bp, Homo sapiens
gccacgctgacggcattcctggg

SEQ ID NO: 1561 - DNA, 33 bp, Homo sapiens
tggcttctcgaatgtggtagccacagactgcag

SEQ ID NO: 1562 - DNA, 26 bp, Homo sapiens
ccccaaccccagcacaaccagccctg

SEQ ID NO: 1563 - DNA, 36 bp, Homo sapiens
acttgacaacctgtaagggattctgaatcgacaggg

SEQ ID NO: 1564 - DNA, 28 bp, Homo sapiens
cacccacttgctaagccagtctcactct

SEQ ID NO: 1565 - DNA, 32 bp, Homo sapiens
acagccagtccctgaggaaggagtcgatgaag

SEQ ID NO: 1566 - DNA, 25 bp, Homo sapiens
gtctgtccgcttccagcggcacggc

SEQ ID NO: 1567 - DNA, 40 bp, Homo sapiens
tttgcaagagtagacaaatcgaagacccgagccggcgctt

SEQ ID NO: 1568 - DNA, 28 bp, Homo sapiens
gccagtgttcccaggcgaatctgaaggc

SEQ ID NO: 1569 - DNA, 37 bp, Homo sapiens
acctggctccttcctgtattatgctcccttcgagtat

SEQ ID NO: 1570 - DNA, 28 bp, Homo sapiens
ccaagatgacttccttccttagcccagc

SEQ ID NO: 1571 - DNA, 36 bp, Homo sapiens
aaataactgctaaaagtgcttcgaactcctgagctc

SEQ ID NO: 1572 - DNA, 27 bp, Homo sapiens
gccccaccgacaacttaccatcctccg

SEQ ID NO: 1573 - DNA, 37 bp, Homo sapiens
tcagtctcacaagccatatttcaagtgctcgagaacc

SEQ ID NO: 1574 - DNA, 24 bp, Homo sapiens
tgaccctccttaaaaaaagaagcc

SEQ ID NO: 1575 - DNA, 40 bp, Homo sapiens
atctatgtaatggacacatcgaactcctgaccttgtgatc

SEQ ID NO: 1576 - DNA, 21 bp, Homo sapiens
cagctccaggatttctaattc

SEQ ID NO: 1577 - DNA, 40 bp, Homo sapiens
aggttctcgcagaaaggctcgaacttgctagacaaagact

SEQ ID NO: 1578 - DNA, 23 bp, Homo sapiens
gtgggaacgggtcagcgtcctgg

SEQ ID NO: 1579 - DNA, 40 bp, Homo sapiens
tttattcatagtgcctggaatgtagtaggatctcgatttg

SEQ ID NO: 1580 - DNA, 22 bp, Homo sapiens
ccaataagaggacgagggtgtg

SEQ ID NO: 1581 - DNA, 40 bp, Homo sapiens
ttggctctaacccacagatcgagagatcaggaaatattta

SEQ ID NO: 1582 - DNA, 25 bp, Homo sapiens
cccagcctgatctcagactcttgtg

SEQ ID NO: 1583 - DNA, 40 bp, Homo sapiens
cgtaaaacgatgatagaatcgaactcctggtctcaagtga

SEQ ID NO: 1584 - DNA, 24 bp, Homo sapiens
ggcttcaagcaatcctcccacctc

SEQ ID NO: 1585 - DNA, 40 bp, Homo sapiens
ttttaagcttagttggaatcgacctttcaggctcaagcaa

SEQ ID NO: 1586 - DNA, 23 bp, Homo sapiens
attctccggaagagatagtgtaa

SEQ ID NO: 1587 - DNA, 40 bp, Homo sapiens; identical to SEQ ID NO: 1503

SEQ ID NO: 1588 - DNA, 28 bp, Homo sapiens
gcccagcagtcaaggttggaagcccagg

SEQ ID NO: 1589 - DNA, 37 bp, Homo sapiens
aggagatttcgatttctgggcttggccattatcaccc

SEQ ID NO: 1590 - DNA, 25 bp, Homo sapiens
tgattctccttcctcagcctcccga

SEQ ID NO: 1591 - DNA, 38 bp, Homo sapiens
tgtttgtcgagaccatgctggctaatatgatgaaaccc

SEQ ID NO: 1592 - DNA, 23 bp, Homo sapiens
cacccaggttggagtgtagtggt

SEQ ID NO: 1593 - DNA, 40 bp, Homo sapiens
atgttggccaggctggtctcgagaccagcctggccaacat

SEQ ID NO: 1594 - DNA, 28 bp, Homo sapiens
agggtttgaccacagacctgagagatga

SEQ ID NO: 1595 - DNA, 36 bp, Homo sapiens
aggatactgcattgtcgactgcatttctgccttcgg

SEQ ID NO: 1596 - DNA, 24 bp, Homo sapiens
ccccggaggagaccctttgaaggt

SEQ ID NO: 1597 - DNA, 40 bp, Homo sapiens
ttcagaaagagggggctctcgaagatccccgggcagcgcg

SEQ ID NO: 1598 - DNA, 28 bp, Homo sapiens
gggaggaagaacagcaccgcccagcgtt

SEQ ID NO: 1599 - DNA, 36 bp, Homo sapiens
tcgtgatgttcgaagcacaaagctcacttactcccg

SEQ ID NO: 1600 - DNA, 24 bp, Homo sapiens
atccttttcaatgaaagggaaatt

SEQ ID NO: 1601 - DNA, 40 bp, Homo sapiens
agagaagaaattaatttttcgacctcccaaagtgctagga

SEQ ID NO: 1602 - DNA, 40 bp, Homo sapiens
attcagatattctacttctcgaagggagcataatacagga

SEQ ID NO: 1603 - DNA, 37 bp, Homo sapiens
tcattctcgatcctcactttccacattagcccagtcc

SEQ ID NO: 1604 - DNA, 40 bp, Homo sapiens
ccgtcctggtgaacacaatcgaaaaaaataaataaaaata

SEQ ID NO: 1605 - DNA, 34 bp, Homo sapiens
tggcccttgaccacaccaagctcgaaattgtcag

SEQ ID NO: 1606 - DNA, 40 bp, Homo sapiens
ctccccaccatcctcctgtcgaaatctctgattagccaaa

SEQ ID NO: 1607 - DNA, 40 bp, Homo sapiens; identical to SEQ ID NO: 1513

SEQ ID NO: 1608 - DNA, 33 bp, Homo sapiens
ttcctcgagttcagcatgcccacaacttctgcc

SEQ ID NO: 1609 - DNA, 37 bp, Homo sapiens
tggcagctcagtcgatattgtcatgaaccatcacctg

SEQ ID NO: 1610 - DNA, 37 bp, Homo sapiens
tgagcatttacatgccctagaattcatcgatcctgcc

SEQ ID NO: 1611 - DNA, 32 bp, Homo sapiens
aggactctcgatgctgtgtgacacagggagct

SEQ ID NO: 1612 - DNA, 40 bp, Homo sapiens
tagttatggaaggctaaatcgacattggattctctctctt

SEQ ID NO: 1613 - DNA, 28 bp, Homo sapiens
cccagcaaggggtccttcgattacaaaa

SEQ ID NO: 1614 - DNA, 40 bp, Homo sapiens
cgagaaatgtgcaaatattcgatcctgccttacttttctt

SEQ ID NO: 1615 - DNA, 40 bp, Homo sapiens
acattagccaggatgttctcgataactagggatgtgagtg

SEQ ID NO: 1616 - DNA, 35 bp, Homo sapiens
tgatgctcgtaacctgttcgaccatacttcagccc

SEQ ID NO: 1617 - DNA, 32 bp, Homo sapiens
tgggtggtctcgaataggcacgtgacccattg

SEQ ID NO: 1618 - DNA, 40 bp, Homo sapiens
tgtggagagggattaacctcgatgggggagggattcaaaa

SEQ ID NO: 1619 - DNA, 38 bp, Homo sapiens
aacatcgaaaccctcctcgtatttgttactctctctac

SEQ ID NO: 1620 - DNA, 40 bp, Homo sapiens
tgtgaatgaccctgggtgtcgatcctgttaggaaacttgg

SEQ ID NO: 1621 - DNA, 40 bp, Homo sapiens
aaacctaaatgatcagtttcgacgtttgcctttgcgtgtg

SEQ ID NO: 1622 - DNA, 32 bp, Homo sapiens
agaccttggatgcaggctcttctcgaggggaa

SEQ ID NO: 1623 - DNA, 37 bp, Homo sapiens
aatctcgatggggtcaataccttatcaggccacagga

SEQ ID NO: 1624 - DNA, 40 bp, Homo sapiens
gagggccagaggatggggtcgaattctctgttgagcaggt

SEQ ID NO: 1625 - DNA, 37 bp, Homo sapiens
atcatcgagttgtgatcctcagtgttggaagaggctc

SEQ ID NO: 1626 - DNA, 37 bp, Homo sapiens
aggtgggaagatcacgaggtcaggagttcgaaaatca

SEQ ID NO: 1627 - DNA, 40 bp, Homo sapiens
attttggccaggctggtctcgatgtaaacagaatggggat

SEQ ID NO: 1628 - DNA, 40 bp, Homo sapiens
taaaataaaacaatattgtcgaatggccagaatggcaaga

SEQ ID NO: 1629 - DNA, 34 bp, Homo sapiens
aggaatcgaggtcaacaaccacttccctcgtgag

SEQ ID NO: 1630 - DNA, 40 bp, Homo sapiens
ccgtagaggctgatgcagtcgaatcagcatttgtataggg

SEQ ID NO: 1631 - DNA, 34 bp, Homo sapiens
tgcagtctgtggctaccacattcgagaagccaag

SEQ ID NO: 1632 - DNA, 36 bp, Homo sapiens
tctcccctgtcgattcagaatcccttacaggttgtc

SEQ ID NO: 1633 - DNA, 32 bp, Homo sapiens
tccttcatcgactccttcctcagggactggct

SEQ ID NO: 1634 - DNA, 40 bp, Homo sapiens
aagcgccggctcgggtcttcgatttgtctactcttgcaaa

SEQ ID NO: 1635 - DNA, 36 bp, Homo sapiens
atactcgaagggagcataatacaggaaggagccagg

SEQ ID NO: 1636 - DNA, 36 bp, Homo sapiens
agctcaggagttcgaagcacttttagcagttattta

SEQ ID NO: 1637 - DNA, 37 bp, Homo sapiens
tggttctcgagcacttgaaatatggcttgtgagactg

SEQ ID NO: 1638 - DNA, 40 bp, Homo sapiens
gatcacaaggtcaggagttcgatgtgtccattacatagat

SEQ ID NO: 1639 - DNA, 40 bp, Homo sapiens
agtctttgtctagcaagttcgagcctttctgcgagaacct

SEQ ID NO: 1640 - DNA, 40 bp, Homo sapiens
acaaatcgagatcctactacattccaggcactatgaataa

SEQ ID NO: 1641 - DNA, 40 bp, Homo sapiens
taaatatttcctgatctctcgatctgtgggttagagccaa

SEQ ID NO: 1642 - DNA, 40 bp, Homo sapiens
tcacttgagaccaggagttcgattctatcatcgttttacg

SEQ ID NO: 1643 - DNA, 40 bp, Homo sapiens
ttgcttgagcctgaaaggtcgattccaactaagcttaaaa

SEQ ID NO: 1644 - DNA, 40 bp, Homo sapiens; identical to SEQ ID NO: 1602

SEQ ID NO: 1645 - DNA, 37 bp, Homo sapiens
agggtgataatggccaagcccagaaatcgaaatctcc

SEQ ID NO: 1646 - DNA, 39 bp, Homo sapiens
tcatattagccagcatggtctcgacaaacaatataacgt

SEQ ID NO: 1647 - DNA, 40 bp, Homo sapiens; identical to SEQ ID NO: 1593

SEQ ID NO: 1648 - DNA, 36 bp, Homo sapiens
tgcagtcgacaatgcagtatccttgcttcctagcac

SEQ ID NO: 1649 - DNA, 40 bp, Homo sapiens
cgcgctgcccggggatcttcgagagccccctctttctgaa

SEQ ID NO: 1650 - DNA, 35 bp, Homo sapiens
tgcttcgaacatcacgaagacattggatgtggccc

SEQ ID NO: 1651 - DNA, 40 bp, Homo sapiens
tcctagcactttgggaggtcgaaaaattaatttcttctct

SEQ ID NO: 1652 - DNA, 60 bp, Homo sapiens
gatagattatggagggttaaggaaggaatcgataacgccaagcactcgggcatgggtgac

SEQ ID NO: 1653 - DNA, 60 bp, Homo sapiens
aaaaccagagaaaggagcaaagtgagcctcgagtacagaggcacagggcagcttttgggg

SEQ ID NO: 1654 - DNA, 60 bp, Homo sapiens
actattactcaaatgttaaagctcttggtcgaagggagcataatacaggaaggagccagg

SEQ ID NO: 1655 - DNA, 60 bp, Homo sapiens
tttctacattttagggagacatgagatatcgagaccaacctgacaacatggcaaaacccc

SEQ ID NO: 1656 - DNA, 60 bp, Homo sapiens
agtggtatgacaatggctcactgcagcctcgaactccacacccctgctaggaagggcaca

SEQ ID NO: 1657 - DNA, 60 bp, Homo sapiens
taattagtgagccacagactctaggccatcgaggctacagtgagctatgatcacgtcact

SEQ ID NO: 1658 - DNA, 60 bp, Homo sapiens
agtttctagagaggattaaggagggggctcgagggcctttgccctttcgtaagggaaaga

SEQ ID NO: 1659 - DNA, 60 bp, Homo sapiens
ttggcttctgactgtatactctgagcactcgaggtgtactgagatatctcgcccccgagg

SEQ ID NO: 1660 - DNA, 60 bp, Homo sapiens
aaaaaccttagactagacggattcacagtcgagacagtgtcttactctgttgtccaggct

SEQ ID NO: 1661 - DNA, 60 bp, Homo sapiens
agtgcaacagaccaatgaagtgagactttcgatataatagtggcaaacaaaatccagcaa

SEQ ID NO: 1662 - DNA, 60 bp, Homo sapiens
ggcagaaaaatgagtgaagtcagagcgatcgagtctgcacctatttaataataactctcc

SEQ ID NO: 1663 - DNA, 60 bp, Homo sapiens
aagcaggtgtccattattaattcattcatcgaactcctggccttatgtaacccacctgcc

SEQ ID NO: 1664 - DNA, 60 bp, Homo sapiens
tcacttagtagccatcacggttatcagatcgaactcctgttctcaagtgatctgcgtgcc

SEQ ID NO: 1665 - DNA, 60 bp, Homo sapiens
atctagtttccatgcaaactcagaagcatcgatttgggtgtgtctgatgtatttccatga

SEQ ID NO: 1666 - DNA, 60 bp, Homo sapiens
ccagcagttctgcaaagacaagcactggtcgattgtggatgggagtgtggattttgtggc

SEQ ID NO: 1667 - DNA, 60 bp, Homo sapiens
ttatgggtaacttcgtgtctttttttgttcgaatttttacacccggcccttgcttccagg

SEQ ID NO: 1668 - DNA, 60 bp, Homo sapiens
ttcaattacctcccactaggtccctccctcgatagcagaatctcccagtctggagcacag

SEQ ID NO: 1669 - DNA, 60 bp, Homo sapiens
ttcatagtgcctggaatgtagtaggatctcgaatcacttcctacctgcaggaggcagaga

SEQ ID NO: 1670 - DNA, 60 bp, Homo sapiens
gggtttccccatgttggccaggttggtctcgagaccagcctggccaacatggcaaaaccc

SEQ ID NO: 1671 - DNA, 60 bp, Homo sapiens
cgttccgggagtaagtgagctttgtgcttcgagccttcttactcaaccactcaacatctg

SEQ ID NO: 1672 - DNA, 60 bp, Homo sapiens
ttccaaggacacaacatgatctcatctttcgaactccagcctgggtaacagagactccat

SEQ ID NO: 1673 - DNA, 60 bp, Homo sapiens
cttctatacgccagaacatggatgaacctcgagataattttgtatgtttccttttttctt

SEQ ID NO: 1674 - DNA, 60 bp, Homo sapiens
cctggctccttcctgtattatgctcccttcgagagaacactatcagagctcccatggccc

SEQ ID NO: 1675 - DNA, 60 bp, Homo sapiens
ttcaagttcccatccaatgagacatgtgtcgagatcagcctggccaacgtggtgaaaccc

SEQ ID NO: 1676 - DNA, 60 bp, Homo sapiens
aaatctggggaccagtagctgtggctagtcgagccactgcactccagcctgcaggacaga

SEQ ID NO: 1677 - DNA, 60 bp, Homo sapiens
tgtgttcattcaagtctttgatcttcactcgaatctgagagacagagagagagcaaggga

SEQ ID NO: 1678 - DNA, 60 bp, Homo sapiens
aatcactaaatttccaaatgtacatggatcgacctcccaggctcaagtggtcttcctgtc

SEQ ID NO: 1679 - DNA, 60 bp, Homo sapiens
gaatgaaggggccctagggcagtggtgttcgagaggcggaagtaaaaggattcagatccc

SEQ ID NO: 1680 - DNA, 60 bp, Homo sapiens
tttgactctagcccagtgattcccaaactcgagtcaaagttgaaaattcatagtaagatt

SEQ ID NO: 1681 - DNA, 60 bp, Homo sapiens
aaaacaaattgagtatttcaagtaaacctcgaaccactgcactctagtccaggcaacaga

SEQ ID NO: 1682 - DNA, 60 bp, Homo sapiens
tgactcacgagggaagtggttgttgacctcgaggagactggaccagaagagtttcccacc

SEQ ID NO: 1683 - DNA, 60 bp, Homo sapiens
gaggtggtgggcaggaaaggaagacagttcgaatggtctgttcaagactattttatagtg

SEQ ID NO: 1684 - DNA, 60 bp, Homo sapiens
ggcaggaggattgcttgagcctaggaaatcgaactcctgggctcaagcaatcctcccgcc

SEQ ID NO: 1685 - DNA, 60 bp, Homo sapiens
ggttgggggatcacttgaggtcaggagctcgaactcctgacctcaagtgatccactctcc

SEQ ID NO: 1686 - DNA, 60 bp, Homo sapiens
cagactttcatattttcaatctctctcttcgatgctcaggaagcgcaggtccgtcagcct

SEQ ID NO: 1687 - DNA, 60 bp, Homo sapiens
aggtttcaccatgttggccaggctggtctcgagaccagcctggccaacatggtgaaaccc

SEQ ID NO: 1688 - DNA, 60 bp, Homo sapiens
atattcctcgcatggagggaacttggggtcgaaatcctggcctcaagcaattcacccacc

SEQ ID NO: 1689 - DNA, 60 bp, Homo sapiens
taagaagtggaagtcgccatgagagtgctcgatacaatggaatgttattccaccataaaa

SEQ ID NO: 1690 - DNA, 60 bp, Homo sapiens
ggtggctacatcattatatggccaagactcgacttttaaattcctaagtttcctagttgg

SEQ ID NO: 1691 - DNA, 60 bp, Homo sapiens
cgcgcactggatcaaaataatacacgcctcgaactaactttccaagcttgactcttagga

SEQ ID NO: 1692 - DNA, 60 bp, Homo sapiens
tttactgaacattggtaaatctacctaatcgaaatacaaaaattagccaggcatgatggc

SEQ ID NO: 1693 - DNA, 60 bp, Homo sapiens
gtgattggtgaacagtggcttcctttagtcgactaaatggaacgtttaaatgagttgaag

SEQ ID NO: 1694 - DNA, 60 bp, Homo sapiens
cgcctttgagtctatttctgacttcatctcgacggaatgtggcccacggcattaaattta

SEQ ID NO: 1695 - DNA, 60 bp, Homo sapiens
gccaggaggatcatttgaggtcaggagttcgaactcctgacctcagatgatccacacacc

SEQ ID NO: 1696 - DNA, 60 bp, Homo sapiens
cctggctccttcctgtattatgctcccttcgaatcttcatcgctatcagtcagaaattct

SEQ ID NO: 1697 - DNA, 60 bp, Homo sapiens
atgcctgtaatcacgggactttgggaggtcgatctcatgacatttaatattactttttgg

SEQ ID NO: 1698 - DNA, 60 bp, Homo sapiens
tttgtttcgggtgctctagaatattccatcgagggccgtggttacagaatcttctggggt

SEQ ID NO: 1699 - DNA, 60 bp, Homo sapiens
tcatgcagaattccaattagcctcttaatcgaactcctgacctcgggggatcggcccgcc

SEQ ID NO: 1700 - DNA, 60 bp, Homo sapiens
gagccgacccttgggagtctgaacatcttcgacttcaaaagccacatagatgatcccctt

SEQ ID NO: 1701 - DNA, 60 bp, Homo sapiens
tctccataaaatatggaggatactgccctcgaactcctgggctccattaatcttctgcct

SEQ ID NO: 1702 - DNA, 23 bp, Homo sapiens
ctgagttacagtggtgggagtgg

SEQ ID NO: 1703 - DNA, 23 bp, Homo sapiens
cttgctcttgtccaccacgccgt

SEQ ID NO: 1704 - DNA, 23 bp, Homo sapiens
gtcaacatcccattgctggtgcc

SEQ ID NO: 1705 - DNA, 23 bp, Homo sapiens
ctccctcttgctgctgggcacta

SEQ ID NO: 1706 - DNA, 25 bp, Homo sapiens
ttgcgttataaaaacatgtataaaa

SEQ ID NO: 1707 - DNA, 25 bp, Homo sapiens
ctggcatccagagctcagtgaacat

SEQ ID NO: 1708 - DNA, 21 bp, Homo sapiens
aatatgagtgaccatggccgc

SEQ ID NO: 1709 - DNA, 21 bp, Homo sapiens
acgatctcggcttactgcaac

SEQ ID NO: 1710 - DNA, 24 bp, Homo sapiens
ggatgcatcccaagggcctatttc

SEQ ID NO: 1711 - DNA, 24 bp, Homo sapiens
catttttccggggaatttttgtgc

SEQ ID NO: 1712 - DNA, 22 bp, Homo sapiens
atagtacacctacatatgctaa

SEQ ID NO: 1713 - DNA, 22 bp, Homo sapiens
ctttatatttattaaggctttt

SEQ ID NO: 1714 - DNA, 26 bp, Homo sapiens
ccctgtctgctatgtgagaggtagtg

SEQ ID NO: 1715 - DNA, 26 bp, Homo sapiens
aggggctgcggagggccgtgtggggc

SEQ ID NO: 1716 - DNA, 23 bp, Homo sapiens
cgccagaaccacaacacacactc

SEQ ID NO: 1717 - DNA, 23 bp, Homo sapiens
tgcctcatactgcggtgtccctc

SEQ ID NO: 1718 - DNA, 23 bp, Homo sapiens
tggaaacataaaacctctcaaga

SEQ ID NO: 1719 - DNA, 23 bp, Homo sapiens
atgtacctgtactcccagttact

SEQ ID NO: 1720 - DNA, 26 bp, Homo sapiens
ctttccatccaaaatggactcactgc

SEQ ID NO: 1721 - DNA, 26 bp, Homo sapiens
ggtttttaatttaaaggaatgttgaa

SEQ ID NO: 1722 - DNA, 23 bp, Homo sapiens
ggagggcagcattcacactggac

SEQ ID NO: 1723 - DNA, 23 bp, Homo sapiens
gggtgcccaggaaaactgtgaac

SEQ ID NO: 1724 - DNA, 26 bp, Homo sapiens
cattttcctccgtgtggtgacttcat

SEQ ID NO: 1725 - DNA, 25 bp, Homo sapiens
aaatgggagcagccacagtgaggac

SEQ ID NO: 1726 - DNA, 23 bp, Homo sapiens
tttgtccagggaacccacactgc

SEQ ID NO: 1727 - DNA, 23 bp, Homo sapiens
cgaggcacagacccctgattgac

SEQ ID NO: 1728 - DNA, 25 bp, Homo sapiens
ggctcttctgtgaggcactctgcca

SEQ ID NO: 1729 - DNA, 25 bp, Homo sapiens
agaactatcaatgccataactctga

SEQ ID NO: 1730 - DNA, 23 bp, Homo sapiens
cctccagacacagcatcttctcc

SEQ ID NO: 1731 - DNA, 23 bp, Homo sapiens
ccccaccctcctcctcaacattc

SEQ ID NO: 1732 - DNA, 23 bp, Homo sapiens
tataatgtgttaggttgaataag

SEQ ID NO: 1733 - DNA, 23 bp, Homo sapiens
agaaagaagaagaagctgatagt

SEQ ID NO: 1734 - DNA, 26 bp, Homo sapiens
gaaggaggaactgtcaaacactcaca

SEQ ID NO: 1735 - DNA, 26 bp, Homo sapiens
cctggctatgtaagtcctctgctgtg

SEQ ID NO: 1736 - DNA, 23 bp, Homo sapiens
ctctcccctgaagttcccacctg

SEQ ID NO: 1737 - DNA, 23 bp, Homo sapiens
catctctgctactcgtgctccca

SEQ ID NO: 1738 - DNA, 22 bp, Homo sapiens
ctcctgagtagttgggattaca

SEQ ID NO: 1739 - DNA, 22 bp, Homo sapiens
tgcctcctaggttcaagcgatt

SEQ ID NO: 1740 - DNA, 22 bp, Homo sapiens
tctgctaagggaacccagcttc

SEQ ID NO: 1741 - DNA, 22 bp, Homo sapiens
aaagtgaaaagacaacccacag

SEQ ID NO: 1742 - DNA, 23 bp, Homo sapiens
ctggagaactgagcagagcgggc

SEQ ID NO: 1743 - DNA, 25 bp, Homo sapiens
ccccttagtctgggattgtagattg

SEQ ID NO: 1744 - DNA, 23 bp, Homo sapiens
gattagaaatacaggggactcca

SEQ ID NO: 1745 - DNA, 23 bp, Homo sapiens
aacactaaaatttctctaagaga

SEQ ID NO: 1746 - DNA, 23 bp, Homo sapiens
ggttggaggcatctctgacacca

SEQ ID NO: 1747 - DNA, 23 bp, Homo sapiens
gcggagaagcctgtgctgcgaat

SEQ ID NO: 1748 - DNA, 24 bp, Homo sapiens
ggctgcccaccctcgtcaggaaaa

SEQ ID NO: 1749 - DNA, 24 bp, Homo sapiens
cctgggttcaagcagttctcctgt

SEQ ID NO: 1750 - DNA, 23 bp, Homo sapiens
ccatagggtctctttgggctgag

SEQ ID NO: 1751 - DNA, 23 bp, Homo sapiens
ctgggagtgcctgacctgttctc

SEQ ID NO: 1752 - DNA, 22 bp, Homo sapiens
tctccaaggcactcacaagtat

SEQ ID NO: 1753 - DNA, 22 bp, Homo sapiens
gaaacgttcatcaggagttgga

SEQ ID NO: 1754 - DNA, 21 bp, Homo sapiens
ctgggtttctgattcatttgg

SEQ ID NO: 1755 - DNA, 21 bp, Homo sapiens
acactttgggaggccaaggct

SEQ ID NO: 1756 - DNA, 21 bp, Homo sapiens
aggggaaacgtggcattcacg

SEQ ID NO: 1757 - DNA, 21 bp, Homo sapiens
gtgtgagctgctccctctacc

SEQ ID NO: 1758 - DNA, 23 bp, Homo sapiens
acagaaaagagagaggaatggca

SEQ ID NO: 1759 - DNA, 23 bp, Homo sapiens
gttttctggataaataaatgtaa

SEQ ID NO: 1760 - DNA, 26 bp, Homo sapiens
ggaccacagggattttcttcaagcac

SEQ ID NO: 1761 - DNA, 23 bp, Homo sapiens
cactaccccacccctcacctttt

SEQ ID NO: 1762 - DNA, 24 bp, Homo sapiens
ctccctgaaatgcagatggaaatg

SEQ ID NO: 1763 - DNA, 24 bp, Homo sapiens
aaattgatactttccacaacaatg

SEQ ID NO: 1764 - DNA, 23 bp, Homo sapiens
gctctgcttctgggaaccctttc

SEQ ID NO: 1765 - DNA, 23 bp, Homo sapiens
caggaagcccagatgctgtgtgg

SEQ ID NO: 1766 - DNA, 23 bp, Homo sapiens
ggtgtggtgcctcacgccttgta

SEQ ID NO: 1767 - DNA, 23 bp, Homo sapiens
gcaggaaggagtctttgagaggc

SEQ ID NO: 1768 - DNA, 23 bp, Homo sapiens
ccttggagtcctggcttcctcta

SEQ ID NO: 1769 - DNA, 24 bp, Homo sapiens
gcagttcagcccacagacagcatc

SEQ ID NO: 1770 - DNA, 23 bp, Homo sapiens
ctgcttgcttgggagtctgtggc

SEQ ID NO: 1771 - DNA, 23 bp, Homo sapiens
tctcaggtccgacgcctccttat

SEQ ID NO: 1772 - DNA, 24 bp, Homo sapiens
caagtttcactcttgttgcccagg

SEQ ID NO: 1773 - DNA, 24 bp, Homo sapiens
gtctcgctctgtcacctacgctgg

SEQ ID NO: 1774 - DNA, 22 bp, Homo sapiens
ccgcgtagcgctgcacggtgca

SEQ ID NO: 1775 - DNA, 22 bp, Homo sapiens
cttccctttcttcgtgtggctt

SEQ ID NO: 1776 - DNA, 23 bp, Homo sapiens
ggcgacagagtgagaccctgtct

SEQ ID NO: 1777 - DNA, 23 bp, Homo sapiens
gccctgtggataaaggggagcta

SEQ ID NO: 1778 - DNA, 26 bp, Homo sapiens
agacaccagagagagagccagaagtg

SEQ ID NO: 1779 - DNA, 26 bp, Homo sapiens
gcttagtggacagtttcagggttcta

SEQ ID NO: 1780 - DNA, 26 bp, Homo sapiens
aagccaagccaagtcacatacggcac

SEQ ID NO: 1781 - DNA, 26 bp, Homo sapiens
ctcctgtcagtaatcccatacattgg

SEQ ID NO: 1782 - DNA, 26 bp, Homo sapiens
aggagtcagagagggccccaagcaca

SEQ ID NO: 1783 - DNA, 26 bp, Homo sapiens
gtggctggagtgcaatggtgctatct

SEQ ID NO: 1784 - DNA, 26 bp, Homo sapiens
cgtgtgctagagaaggtgacaggtca

SEQ ID NO: 1785 - DNA, 26 bp, Homo sapiens
cagttattcattcaccagaaatgtgt

SEQ ID NO: 1786 - DNA, 26 bp, Homo sapiens
caaatctcttcttgctcaaccgcagc

SEQ ID NO: 1787 - DNA, 26 bp, Homo sapiens
cttaggaaggttttcagacaccaggc

SEQ ID NO: 1788 - DNA, 23 bp, Homo sapiens
tggactaccttagagccacaaaa

SEQ ID NO: 1789 - DNA, 23 bp, Homo sapiens
tgtaaccccagcactttaggatg

SEQ ID NO: 1790 - DNA, 21 bp, Homo sapiens
ggctctgggcctgaggctggc

SEQ ID NO: 1791 - DNA, 21 bp, Homo sapiens
tggtggtgcgtgtctgtaatc

SEQ ID NO: 1792 - DNA, 24 bp, Homo sapiens
cagtctttttatcactcaatttgt

SEQ ID NO: 1793 - DNA, 24 bp, Homo sapiens
tgattgctgatagctaaacgtatt

SEQ ID NO: 1794 - DNA, 26 bp, Homo sapiens
cccatccgccacccgtgactggggca

SEQ ID NO: 1795 - DNA, 26 bp, Homo sapiens
tgcaaacccttccctgctttctgcgt

SEQ ID NO: 1796 - DNA, 21 bp, Homo sapiens
gctaattgtttgtattttagt

SEQ ID NO: 1797 - DNA, 21 bp, Homo sapiens
gtagattcaaagaaagcaaaa

SEQ ID NO: 1798 - DNA, 23 bp, Homo sapiens
tactcgccaggcagtgattccac

SEQ ID NO: 1799 - DNA, 23 bp, Homo sapiens
gagggcacaggacagaggtcaca

SEQ ID NO: 1800 - DNA, 23 bp, Homo sapiens
gacatccttgcctctgccttggg

SEQ ID NO: 1801 - DNA, 23 bp, Homo sapiens
gtgggtggtagatgatagagggc

SEQ ID NO: 1802 - DNA, 28 bp, Homo sapiens
ctgaggaacagtggaagatgaggctggg

SEQ ID NO: 1803 - DNA, 23 bp, Homo sapiens
gggtgtccttggcagtggtgggc

SEQ ID NO: 1804 - DNA, 23 bp, Homo sapiens
ttaaatattcttagaaaaaagtt

SEQ ID NO: 1805 - DNA, 23 bp, Homo sapiens
caaatatgagtgaccatggccgc

SEQ ID NO: 1806 - DNA, 23 bp, Homo sapiens
aagggcctatttcctcgttctag

SEQ ID NO: 1807 - DNA, 25 bp, Homo sapiens
cattattacagctggcaaatcaaat

SEQ ID NO: 1808 - DNA, 21 bp, Homo sapiens
tgagaggtagtgacaggactt

SEQ ID NO: 1809 - DNA, 28 bp, Homo sapiens
ctctgttctccaagcacctcccaccagg

SEQ ID NO: 1810 - DNA, 26 bp, Homo sapiens
aagaaatggaaaccctgaacaaacca

SEQ ID NO: 1811 - DNA, 26 bp, Homo sapiens
ctgctcctcactttccatccaaaatg

SEQ ID NO: 1812 - DNA, 28 bp, Homo sapiens
gccagacacagaggatgaggagagcagc

SEQ ID NO: 1813 - DNA, 28 bp, Homo sapiens
tacattttcctccgtgtggtgacttcat

SEQ ID NO: 1814 - DNA, 26 bp, Homo sapiens
gcaacactcggctccatcccacttgg

SEQ ID NO: 1815 - DNA, 24 bp, Homo sapiens
aaatacgagcaagtgatagtccgg

SEQ ID NO: 1816 - DNA, 28 bp, Homo sapiens
gagggagggagagaagggaggaaagggt

SEQ ID NO: 1817 - DNA, 21 bp, Homo sapiens
atgtagataaaatcacaaatg

SEQ ID NO: 1818 - DNA, 23 bp, Homo sapiens
cgcaaccaggagtctgcctacat

SEQ ID NO: 1819 - DNA, 28 bp, Homo sapiens
acccctctcccactccactgcctctcag

SEQ ID NO: 1820 - DNA, 24 bp, Homo sapiens
ctcagcctcctgagtagttgggat

SEQ ID NO: 1821 - DNA, 23 bp, Homo sapiens
gcaccgatgttcacagtctaccc

SEQ ID NO: 1822 - DNA, 26 bp, Homo sapiens
cgccctggagaactgagcagagcggg

SEQ ID NO: 1823 - DNA, 26 bp, Homo sapiens
actccatgttcatagtggcgttattc

SEQ ID NO: 1824 - DNA, 27 bp, Homo sapiens
cccacccaggttggaggcatctctgac

SEQ ID NO: 1825 - DNA, 26 bp, Homo sapiens
tggagggtgcaggagaaggaggggct

SEQ ID NO: 1826 - DNA, 28 bp, Homo sapiens
gccacgctgcccctgctgatgtctttgc

SEQ ID NO: 1827 - DNA, 25 bp, Homo sapiens
gggtggggtgcctggatgcgtgagg

SEQ ID NO: 1828 - DNA, 24 bp, Homo sapiens
ccctttttatgtctgggtttctga

SEQ ID NO: 1829 - DNA, 23 bp, Homo sapiens
gtaagaacctgcaaatgcatgcc

SEQ ID NO: 1830 - DNA, 21 bp, Homo sapiens
aatctagggtgtaaataatac

SEQ ID NO: 1831 - DNA, 28 bp, Homo sapiens
gcaaggaccacagggattttcttcaagc

SEQ ID NO: 1832 - DNA, 23 bp, Homo sapiens
gtgctgccatccgcaggcttaaa

SEQ ID NO: 1833 - DNA, 28 bp, Homo sapiens
cctctctgcctatccttcttcccagggc

SEQ ID NO: 1834 - DNA, 27 bp, Homo sapiens
gccaggtgtggtgcctcacgccttgta

SEQ ID NO: 1835 - DNA, 28 bp, Homo sapiens
cttctatccttggagtcctggcttcctc

SEQ ID NO: 1836 - DNA, 26 bp, Homo sapiens
gacctgcttgcttgggagtctgtggc

SEQ ID NO: 1837 - DNA, 25 bp, Homo sapiens
ttctgcctcccaggttcaaggaatt

SEQ ID NO: 1838 - DNA, 23 bp, Homo sapiens
gcgggggcccagggtcccggcag

SEQ ID NO: 1839 - DNA, 21 bp, Homo sapiens
tgagcagagattgcaccactg

SEQ ID NO: 1840 - DNA, 28 bp, Homo sapiens
cagacaccagagagagagccagaagtgg

SEQ ID NO: 1841 - DNA, 28 bp, Homo sapiens
ttccatcccgcacaggtgagccgtctgg

SEQ ID NO: 1842 - DNA, 25 bp, Homo sapiens
cctaggcagcttaggcagctcacat

SEQ ID NO: 1843 - DNA, 23 bp, Homo sapiens
ggtgacaggtcatgaggaagaga

SEQ ID NO: 1844 - DNA, 28 bp, Homo sapiens
gggcacagtcataagccaggaaatccct

SEQ ID NO: 1845 - DNA, 24 bp, Homo sapiens
gaattggactaccttagagccaca

SEQ ID NO: 1846 - DNA, 26 bp, Homo sapiens
ccaagtcctgcatgagatgaaccagg

SEQ ID NO: 1847 - DNA, 26 bp, Homo sapiens
aaatagctaatactaaataaggacat

SEQ ID NO: 1848 - DNA, 21 bp, Homo sapiens
cttgcccacggccccatccgc

SEQ ID NO: 1849 - DNA, 23 bp, Homo sapiens
ttttagtagagatggggtttcac

SEQ ID NO: 1850 - DNA, 27 bp, Homo sapiens
ggcattgacctcatactcgccaggcag

SEQ ID NO: 1851 - DNA, 23 bp, Homo sapiens
tcaagagccagagcccaggcggg

SEQ ID NO: 1852 - DNA, 27 bp, Homo sapiens
gaacgcttgctcttgtccaccacgccg

SEQ ID NO: 1853 - DNA, 32 bp, Homo sapiens
aggaaggaatcgataacgccaagcactcgggc

SEQ ID NO: 1854 - DNA, 28 bp, Homo sapiens
gctctgctccctcttgctgctgggcact

SEQ ID NO: 1855 - DNA, 32 bp, Homo sapiens
aggagcaaagtgagcctcgagtacagaggcac

SEQ ID NO: 1856 - DNA, 23 bp, Homo sapiens
ctgaggctggcatccagagctca

SEQ ID NO: 1857 - DNA, 40 bp, Homo sapiens
aaatgttaaagctcttggtcgaagggagcataatacagga

SEQ ID NO: 1858 - DNA, 23 bp, Homo sapiens
ccgcctccaaggttcaagcgatt

SEQ ID NO: 1859 - DNA, 40 bp, Homo sapiens
ttagggagacatgagatatcgagaccaacctgacaacatg

SEQ ID NO: 1860 - DNA, 23 bp, Homo sapiens
atttttccggggaatttttgtgc

SEQ ID NO: 1861 - DNA, 40 bp, Homo sapiens
caatggctcactgcagcctcgaactccacacccctgctag

SEQ ID NO: 1862 - DNA, 25 bp, Homo sapiens
aggcttttttgtgcctgattatatg

SEQ ID NO: 1863 - DNA, 40 bp, Homo sapiens
gccacagactctaggccatcgaggctacagtgagctatga

SEQ ID NO: 1864 - DNA, 21 bp, Homo sapiens
ctccggcccactcccctcatg

SEQ ID NO: 1865 - DNA, 40 bp, Homo sapiens
gaggattaaggagggggctcgagggcctttgccctttcgt

SEQ ID NO: 1866 - DNA, 27 bp, Homo sapiens
tctgcctcatactgcggtgtccctcgg

SEQ ID NO: 1867 - DNA, 35 bp, Homo sapiens
tggcttctgactgtatactctgagcactcgaggtg

SEQ ID NO: 1868 - DNA, 26 bp, Homo sapiens
ccaagagtctgagaccagctggggca

SEQ ID NO: 1869 - DNA, 40 bp, Homo sapiens
gactagacggattcacagtcgagacagtgtcttactctgt

SEQ ID NO: 1870 - DNA, 26 bp, Homo sapiens
tcaaagatctattctgagtctactga

SEQ ID NO: 1871 - DNA, 40 bp, Homo sapiens
accaatgaagtgagactttcgatataatagtggcaaacaa

SEQ ID NO: 1872 - DNA, 27 bp, Homo sapiens
cccctgctggtgaagtccccaagaagg

SEQ ID NO: 1873 - DNA, 32 bp, Homo sapiens
atgagtgaagtcagagcgatcgagtctgcacc

SEQ ID NO: 1874 - DNA, 28 bp, Homo sapiens
taaatgggagcagccacagtgaggacca

SEQ ID NO: 1875 - DNA, 37 bp, Homo sapiens
tcattcatcgaactcctggccttatgtaacccacctg

SEQ ID NO: 1876 - DNA, 28 bp, Homo sapiens
cctccctcctctctcccctctggaatgg

SEQ ID NO: 1877 - DNA, 38 bp, Homo sapiens
agtagccatcacggttatcagatcgaactcctgttctc

SEQ ID NO: 1878 - DNA, 24 bp, Homo sapiens
tacaatggagaaatccagcagaaa

SEQ ID NO: 1879 - DNA, 40 bp, Homo sapiens
catgcaaactcagaagcatcgatttgggtgtgtctgatgt

SEQ ID NO: 1880 - DNA, 27 bp, Homo sapiens
gctggagggcaagaactgagggactgc

SEQ ID NO: 1881 - DNA, 33 bp, Homo sapiens
acaagcactggtcgattgtggatgggagtgtgg

SEQ ID NO: 1882 - DNA, 21 bp, Homo sapiens
tccttgagggaaaagtcactg

SEQ ID NO: 1883 - DNA, 40 bp, Homo sapiens
cttcgtgtctttttttgttcgaatttttacacccggccct

SEQ ID NO: 1884 - DNA, 28 bp, Homo sapiens
tccctggctatgtaagtcctctgctgtg

SEQ ID NO: 1885 - DNA, 33 bp, Homo sapiens
aattacctcccactaggtccctccctcgatagc

SEQ ID NO: 1886 - DNA, 24 bp, Homo sapiens
gcctcgggcaggtctcttgctctg

SEQ ID NO: 1887 - DNA, 36 bp, Homo sapiens
agtgcctggaatgtagtaggatctcgaatcacttcc

SEQ ID NO: 1888 - DNA, 24 bp, Homo sapiens
ctagagtgcggtggtgcagcctca

SEQ ID NO: 1889 - DNA, 40 bp, Homo sapiens; identical to SEQ ID NO: 949

SEQ ID NO: 1890 - DNA, 23 bp, Homo sapiens
tgtgtatgataaggggcttgtat

SEQ ID NO: 1891 - DNA, 40 bp, Homo sapiens
gtaagtgagctttgtgcttcgagccttcttactcaaccac

SEQ ID NO: 1892 - DNA, 28 bp, Homo sapiens
gggttggagaccacagtgacaggacagt

SEQ ID NO: 1893 - DNA, 36 bp, Homo sapiens
tctcatctttcgaactccagcctgggtaacagagac

SEQ ID NO: 1894 - DNA, 26 bp, Homo sapiens
ttctctaagagaaccttctactttct

SEQ ID NO: 1895 - DNA, 40 bp, Homo sapiens
ccagaacatggatgaacctcgagataattttgtatgtttc

SEQ ID NO: 1896 - DNA, 28 bp, Homo sapiens
ggagagcaagagcaaaggcggagaagcc

SEQ ID NO: 1897 - DNA, 33 bp, Homo sapiens
tgctcccttcgagagaacactatcagagctccc

SEQ ID NO: 1898 - DNA, 26 bp, Homo sapiens
gctggactgcagcggcgcaacctctg

SEQ ID NO: 1899 - DNA, 40 bp, Homo sapiens
catccaatgagacatgtgtcgagatcagcctggccaacgt

SEQ ID NO: 1900 - DNA, 25 bp, Homo sapiens
gctgggagtgcctgacctgttctca

SEQ ID NO: 1901 - DNA, 30 bp, Homo sapiens
tggctagtcgagccactgcactccagcctg

SEQ ID NO: 1902 - DNA, 25 bp, Homo sapiens
ctctctctccctctctttccctctc

SEQ ID NO: 1903 - DNA, 40 bp, Homo sapiens
caagtctttgatcttcactcgaatctgagagacagagaga

SEQ ID NO: 1904 - DNA, 24 bp, Homo sapiens
tgcagtggctcatgcccataatcc

SEQ ID NO: 1905 - DNA, 40 bp, Homo sapiens
tttccaaatgtacatggatcgacctcccaggctcaagtgg

SEQ ID NO: 1906 - DNA, 23 bp, Homo sapiens
tccaggctgtgtgggatccttgg

SEQ ID NO: 1907 - DNA, 40 bp, Homo sapiens
gccctagggcagtggtgttcgagaggcggaagtaaaagga

SEQ ID NO: 1908 - DNA, 21 bp, Homo sapiens
taaataaatgtaatatcacta

SEQ ID NO: 1909 - DNA, 40 bp, Homo sapiens
gcccagtgattcccaaactcgagtcaaagttgaaaattca

SEQ ID NO: 1910 - DNA, 28 bp, Homo sapiens
gagcacaaaggcaccccagttctccctg

SEQ ID NO: 1911 - DNA, 34 bp, Homo sapiens
agtaaacctcgaaccactgcactctagtccaggc

SEQ ID NO: 1912 - DNA, 23 bp, Homo sapiens
ttgtaaattgatactttccacaa

SEQ ID NO: 1913 - DNA, 40 bp, Homo sapiens
gggaagtggttgttgacctcgaggagactggaccagaaga

SEQ ID NO: 1914 - DNA, 27 bp, Homo sapiens
caggaagcccagatgctgtgtggtttt

SEQ ID NO: 1915 - DNA, 34 bp, Homo sapiens
tggtgggcaggaaaggaagacagttcgaatggtc

SEQ ID NO: 1916 - DNA, 28 bp, Homo sapiens
gggcaggaaggagtctttgagaggctgg

SEQ ID NO: 1917 - DNA, 33 bp, Homo sapiens
tgcttgagcctaggaaatcgaactcctgggctc

SEQ ID NO: 1918 - DNA, 28 bp, Homo sapiens
gcagttcagcccacagacagcatcttct

SEQ ID NO: 1919 - DNA, 33 bp, Homo sapiens
aggagctcgaactcctgacctcaagtgatccac

SEQ ID NO: 1920 - DNA, 28 bp, Homo sapiens
ctcccctctcaggtccgacgcctcctta

SEQ ID NO: 1921 - DNA, 32 bp, Homo sapiens
tctctctcttcgatgctcaggaagcgcaggtc

SEQ ID NO: 1922 - DNA, 25 bp, Homo sapiens
tggagtgcagtggtgcgatctcggc

SEQ ID NO: 1923 - DNA, 40 bp, Homo sapiens; identical to SEQ ID NO: 1593

SEQ ID NO: 1924 - DNA, 23 bp, Homo sapiens
cttccctttcttcgtgtggcttc

SEQ ID NO: 1925 - DNA, 40 bp, Homo sapiens
catggagggaacttggggtcgaaatcctggcctcaagcaa

SEQ ID NO: 1926 - DNA, 21 bp, Homo sapiens
attatctgccatttcaggcct

SEQ ID NO: 1927 - DNA, 40 bp, Homo sapiens
aagtcgccatgagagtgctcgatacaatggaatgttattc

SEQ ID NO: 1928 - DNA, 28 bp, Homo sapiens
ctggcttagtggacagtttcagggttct

SEQ ID NO: 1929 - DNA, 37 bp, Homo sapiens
tggtggctacatcattatatggccaagactcgacttt

SEQ ID NO: 1930 - DNA, 26 bp, Homo sapiens
gctcaacccgtgaccacagcatctgc

SEQ ID NO: 1931 - DNA, 37 bp, Homo sapiens
acgcgcactggatcaaaataatacacgcctcgaacta

SEQ ID NO: 1932 - DNA, 25 bp, Homo sapiens
ttggagatggagtctcgctttgtca

SEQ ID NO: 1933 - DNA, 40 bp, Homo sapiens
attggtaaatctacctaatcgaaatacaaaaattagccag

SEQ ID NO: 1934 - DNA, 23 bp, Homo sapiens
ggggaaaaaaaggctgtattctg

SEQ ID NO: 1935 - DNA, 40 bp, Homo sapiens
aacagtggcttcctttagtcgactaaatggaacgtttaaa

SEQ ID NO: 1936 - DNA, 25 bp, Homo sapiens
ggcagccctcgggagaacaggttgc

SEQ ID NO: 1937 - DNA, 32 bp, Homo sapiens
tctgacttcatctcgacggaatgtggcccacg

SEQ ID NO: 1938 - DNA, 24 bp, Homo sapiens
actttaggatgctgaggtgggagg

SEQ ID NO: 1939 - DNA, 40 bp, Homo sapiens
tcatttgaggtcaggagttcgaactcctgacctcagatga

SEQ ID NO: 1940 - DNA, 26 bp, Homo sapiens
acttcaacccaggaggaggaggttgc

SEQ ID NO: 1941 - DNA, 40 bp, Homo sapiens
tcctgtattatgctcccttcgaatcttcatcgctatcagt

SEQ ID NO: 1942 - DNA, 26 bp, Homo sapiens
tacctggggacaatagtcaatagatt

SEQ ID NO: 1943 - DNA, 40 bp, Homo sapiens
tcacgggactttgggaggtcgatctcatgacatttaatat

SEQ ID NO: 1944 - DNA, 21 bp, Homo sapiens
tactccctttgcaaacccttc

SEQ ID NO: 1945 - DNA, 40 bp, Homo sapiens
gtgctctagaatattccatcgagggccgtggttacagaat

SEQ ID NO: 1946 - DNA, 23 bp, Homo sapiens
caaaacaaaatgagaggatgagg

SEQ ID NO: 1947 - DNA, 40 bp, Homo sapiens
ttccaattagcctcttaatcgaactcctgacctcggggga

SEQ ID NO: 1948 - DNA, 28 bp, Homo sapiens
ggcggagggcacaggacagaggtcacat

SEQ ID NO: 1949 - DNA, 37 bp, Homo sapiens
acccttgggagtctgaacatcttcgacttcaaaagcc

SEQ ID NO: 1950 - DNA, 28 bp, Homo sapiens
cgccctgctggtgaggtcttgctggaag

SEQ ID NO: 1951 - DNA, 32 bp, Homo sapiens
atatggaggatactgccctcgaactcctgggc

SEQ ID NO: 1952 - DNA, 32 bp, Homo sapiens
tgcccgagtgcttggcgttatcgattccttcc

SEQ ID NO: 1953 - DNA, 32 bp, Homo sapiens
tgtgcctctgtactcgaggctcactttgctcc

SEQ ID NO: 1954 - DNA, 40 bp, Homo sapiens
tcctgtattatgctcccttcgaccaagagctttaacattt

SEQ ID NO: 1955 - DNA, 40 bp, Homo sapiens
catgttgtcaggttggtctcgatatctcatgtctccctaa

SEQ ID NO: 1956 - DNA, 40 bp, Homo sapiens
ctagcaggggtgtggagttcgaggctgcagtgagccattg

SEQ ID NO: 1957 - DNA, 40 bp, Homo sapiens
tcatagctcactgtagcctcgatggcctagagtctgtggc

SEQ ID NO: 1958 - DNA, 40 bp, Homo sapiens
acgaaagggcaaaggccctcgagccccctccttaatcctc

SEQ ID NO: 1959 - DNA, 35 bp, Homo sapiens
acacctcgagtgctcagagtatacagtcagaagcc

SEQ ID NO: 1960 - DNA, 40 bp, Homo sapiens
acagagtaagacactgtctcgactgtgaatccgtctagtc

SEQ ID NO: 1961 - DNA, 40 bp, Homo sapiens
ttgtttgccactattatatcgaaagtctcacttcattggt

SEQ ID NO: 1962 - DNA, 32 bp, Homo sapiens
aggtgcagactcgatcgctctgacttcactca

SEQ ID NO: 1963 - DNA, 35 bp, Homo sapiens
aggtgggttacataaggccaggagttcgatgaatg

SEQ ID NO: 1964 - DNA, 38 bp, Homo sapiens
tgagaacaggagttcgatctgataaccgtgatggctac

SEQ ID NO: 1965 - DNA, 40 bp, Homo sapiens
acatcagacacacccaaatcgatgcttctgagtttgcatg

SEQ ID NO: 1966 - DNA, 33 bp, Homo sapiens
tccacactcccatccacaatcgaccagtgcttg

SEQ ID NO: 1967 - DNA, 40 bp, Homo sapiens
agggccgggtgtaaaaattcgaacaaaaaaagacacgaag

SEQ ID NO: 1968 - DNA, 32 bp, Homo sapiens
tgctatcgagggagggacctagtgggaggtaa

SEQ ID NO: 1969 - DNA, 36 bp, Homo sapiens
aggaagtgattcgagatcctactacattccaggcac

SEQ ID NO: 1970 - DNA, 40 bp, Homo sapiens; identical to SEQ ID NO: 898

SEQ ID NO: 1971 - DNA, 40 bp, Homo sapiens
gtggttgagtaagaaggctcgaagcacaaagctcacttac

SEQ ID NO: 1972 - DNA, 36 bp, Homo sapiens
agtctctgttacccaggctggagttcgaaagatgag

SEQ ID NO: 1973 - DNA, 40 bp, Homo sapiens
gaaacatacaaaattatctcgaggttcatccatgttctgg

SEQ ID NO: 1974 - DNA, 33 bp, Homo sapiens
tgggagctctgatagtgttctctcgaagggagc

SEQ ID NO: 1975 - DNA, 40 bp, Homo sapiens
acgttggccaggctgatctcgacacatgtctcattggatg

SEQ ID NO: 1976 - DNA, 31 bp, Homo sapiens
tggctcgactagccacagctactggtcccca

SEQ ID NO: 1977 - DNA, 40 bp, Homo sapiens
tctctctgtctctcagattcgagtgaagatcaaagacttg

SEQ ID NO: 1978 - DNA, 40 bp, Homo sapiens
ccacttgagcctgggaggtcgatccatgtacatttggaaa

SEQ ID NO: 1979 - DNA, 40 bp, Homo sapiens
tccttttacttccgcctctcgaacaccactgccctagggc

SEQ ID NO: 1980 - DNA, 40 bp, Homo sapiens
tgaattttcaactttgactcgagtttgggaatcactgggc

SEQ ID NO: 1981 - DNA, 34 bp, Homo sapiens
tgcctggactagagtgcagtggttcgaggtttac

SEQ ID NO: 1982 - DNA, 40 bp, Homo sapiens
tcttctggtccagtctcctcgaggtcaacaaccacttccc

SEQ ID NO: 1983 - DNA, 34 bp, Homo sapiens
accattcgaactgtcttcctttcctgcccaccac

SEQ ID NO: 1984 - DNA, 33 bp, Homo sapiens
tgcttgagcccaggagttcgatttcctaggctc

SEQ ID NO: 1985 - DNA, 32 bp, Homo sapiens
aggagttcgagctcctgacctcaagtgatccc

SEQ ID NO: 1986 - DNA, 34 bp, Homo sapiens
acctgcgcttcctgagcatcgaagagagagattg

SEQ ID NO: 1987 - DNA, 40 bp, Homo sapiens; identical to SEQ ID NO: 1593

SEQ ID NO: 1988 - DNA, 40 bp, Homo sapiens
ttgcttgaggccaggatttcgaccccaagttccctccatg

SEQ ID NO: 1989 - DNA, 40 bp, Homo sapiens
gaataacattccattgtatcgagcactctcatggcgactt

SEQ ID NO: 1990 - DNA, 38 bp, Homo sapiens
aaagtcgagtcttggccatataatgatgtagccaccac

SEQ ID NO: 1991 - DNA, 38 bp, Homo sapiens
tagttcgaggcgtgtattattttgatccagtgcgcgtg

SEQ ID NO: 1992 - DNA, 40 bp, Homo sapiens
ctggctaatttttgtatttcgattaggtagatttaccaat

SEQ ID NO: 1993 - DNA, 40 bp, Homo sapiens
tttaaacgttccatttagtcgactaaaggaagccactgtt

SEQ ID NO: 1994 - DNA, 32 bp, Homo sapiens
atgccgtgggccacattccgtcgagatgaagt

SEQ ID NO: 1995 - DNA, 40 bp, Homo sapiens
tcatctgaggtcaggagttcgaactcctgacctcaaatga

SEQ ID NO: 1996 - DNA, 40 bp, Homo sapiens
actgatagcgatgaagattcgaagggagcataatacagga

SEQ ID NO: 1997 - DNA, 40 bp, Homo sapiens
atattaaatgtcatgagatcgacctcccaaagtcccgtga

SEQ ID NO: 1998 - DNA, 40 bp, Homo sapiens
attctgtaaccacggccctcgatggaatattctagagcac

SEQ ID NO: 1999 - DNA, 40 bp, Homo sapiens
tcccccgaggtcaggagttcgattaagaggctaattggaa

SEQ ID NO: 2000 - DNA, 34 bp, Homo sapiens
tgaagtcgaagatgttcagactcccaagggtcgg

SEQ ID NO: 2001 - DNA, 32 bp, Homo sapiens
agcccaggagttcgagggcagtatcctccata

SEQ ID NO: 2002 - DNA, 60 bp, Homo sapiens
cctccatttccacccccacgctcaaacctcgacttagaactgtaattgaatttgttataa

SEQ ID NO: 2003 - DNA, 60 bp, Homo sapiens
atgaagatgagcaacttgtctatcttgttcgactcactgcaacccccgcctctgggttct

SEQ ID NO: 2004 - DNA, 60 bp, Homo sapiens
agagaatgggacccactggcactggtgatcgactgcgccccgctgctgttcctagacgca

SEQ ID NO: 2005 - DNA, 60 bp, Homo sapiens
cggggcaaacataaagaaggatttagtgtcgaactcctgacttcaggtgatccacccacc

SEQ ID NO: 2006 - DNA, 60 bp, Homo sapiens; identical to SEQ ID NO: 324

SEQ ID NO: 2007 - DNA, 60 bp, Homo sapiens
ccccatacctgctccaagtctttccatctcgaagtatcctccttaggaaaacccactgga

SEQ ID NO: 2008 - DNA, 60 bp, Homo sapiens
ctctgtatttggaagctgctctaaggtttcgaatccctagcagccagtccctgctacttc

SEQ ID NO: 2009 - DNA, 60 bp, Homo sapiens
agatctggtctatttggttctttaaacctcgaggctcatgcaggctaactgaggcacctc

SEQ ID NO: 2010 - DNA, 60 bp, Homo sapiens
atatatgatttctgaataatctttttgctcgagagggtttggcccccagcgcacctctgc

SEQ ID NO: 2011 - DNA, 60 bp, Homo sapiens
gtggaaggtggaggagggtggaggagggtcgagctccactcagctgggttcagacaagct

SEQ ID NO: 2012 - DNA, 60 bp, Homo sapiens
ggcgcccccgttcaatcagcctgcattgtcgaactcctaagctcaggtgatccacctgcc

SEQ ID NO: 2013 - DNA, 60 bp, Homo sapiens
acatctcccttcggcccctgtggccggctcgatttcaagaagttggtgaaagcagtgttg

SEQ ID NO: 2014 - DNA, 60 bp, Homo sapiens
atcactccctgggcaaccaccccagggctcgacaaggtcaaatgcacagtattatgtcta

SEQ ID NO: 2015 - DNA, 60 bp, Homo sapiens
atctcttgtcggaattattgcagtagtctcgattactttttcgtcttgggctgataatct

SEQ ID NO: 2016 - DNA, 60 bp, Homo sapiens
ctggacttgatagttttaatggaatatgtcgaggctcatgcaggctaactgaggcacctc

SEQ ID NO: 2017 - DNA, 60 bp, Homo sapiens
ccccatgccatcctcccccctctacaattcgagaagacagtccagagctgaacccacccc

SEQ ID NO: 2018 - DNA, 60 bp, Homo sapiens
tttgcctgagtttttaaagcatttttcttcgagccctaccaacccagacagctccttccc

SEQ ID NO: 2019 - DNA, 60 bp, Homo sapiens
acaaattgctcaaccaaaattcatatcatcgacaggcacctttccaggctcttccaagag

SEQ ID NO: 2020 - DNA, 60 bp, Homo sapiens
cagcctgggcaatacagtgagacccatctcgaagtctgaggtaaaacacagaacaggatt

SEQ ID NO: 2021 - DNA, 60 bp, Homo sapiens
gagagggagggaggagggagaagggaggtcgaactcccgacctcaggtgatttgcctgcc

SEQ ID NO: 2022 - DNA, 60 bp, Homo sapiens
caccctttaccatgtttgcaggagatgatcgaacttctgacctcaggtgatctgcccacc

SEQ ID NO: 2023 - DNA, 60 bp, Homo sapiens
atgtagcccaggctggttgtcttttttttcgatccacccatccacccatcctcccatcca

SEQ ID NO: 2024 - DNA, 60 bp, Homo sapiens
cagtgggtcaacttggcacatggaggcctcgacacttcctcagacgctctgagaggtgaa

SEQ ID NO: 2025 - DNA, 60 bp, Homo sapiens
ctcctcctttgtaataagggcactgaattcgatctcagactccatcagagcacagcagcc

SEQ ID NO: 2026 - DNA, 60 bp, Homo sapiens
tcctccctactgcctcccaggctcaaaatcgatataattcatgctgagttctttaagtac

SEQ ID NO: 2027 - DNA, 60 bp, Homo sapiens
tatggtctcagcaaagcgaaggagaaagtcgatccacccatccacccatcctcccatcca

SEQ ID NO: 2028 - DNA, 60 bp, Homo sapiens
cagtcttttgtggagaaatacttgtgactcgatcccttactcctgaggcaactgtgacaa

SEQ ID NO: 2029 - DNA, 60 bp, Homo sapiens
acagattaaaactatgaggatataccattcgagaccagcctgagcaacatagtgagatcc

SEQ ID NO: 2030 - DNA, 60 bp, Homo sapiens
agtatgagccactctcatttttagcccctcgagcgacggaggcgacgcggcggctaccgg

SEQ ID NO: 2031 - DNA, 60 bp, Homo sapiens
ggcatgcatatcgcttgagggcaggagttcgagatcggacacctccaactcccggtctgg

SEQ ID NO: 2032 - DNA, 60 bp, Homo sapiens
accctcctcccatcccccacctggggactcgaactcctgaacttgcgatctgcccgcgtt

SEQ ID NO: 2033 - DNA, 60 bp, Homo sapiens
tttctctcttgtggcccctctggactcctcgaccatatcagtgagtggagtctatggtga

SEQ ID NO: 2034 - DNA, 60 bp, Homo sapiens
gtttgtatctatttctgtgcagaagtcctcgaactcctgacctcaggtcatcctctcact

SEQ ID NO: 2035 - DNA, 60 bp, Homo sapiens
attatttgcccactcccttccacttcaatcgaagatttttctatttatctctgatgtagt

SEQ ID NO: 2036 - DNA, 60 bp, Homo sapiens
ttatttatttttttgtagagacaaggtctcgactctccacgcatctctgccacccgagtg

SEQ ID NO: 2037 - DNA, 60 bp, Homo sapiens
agctggttctttgaacagatcaataaaatcgaatcttcgcggactccacgcatctgtgtt

SEQ ID NO: 2038 - DNA, 60 bp, Homo sapiens
ccctaagtgatacttgggtcatctttcttcgaggagtccagaggggccacaagagagaaa

SEQ ID NO: 2039 - DNA, 60 bp, Homo sapiens
aaaatttcataaaattcaaattttagtgtcgaactcctaacctcaggtgatccgtctgcc

SEQ ID NO: 2040 - DNA, 60 bp, Homo sapiens
ttcacttggactaccctctccgtccacctcgaattctagcaggttttattgtaaaattga

SEQ ID NO: 2041 - DNA, 60 bp, Homo sapiens
ccaaagagctgagacaaaagtacaatgttcgattgaagtggaagggagtgggcaaataat

SEQ ID NO: 2042 - DNA, 60 bp, Homo sapiens
tctaatctgtggccctggaaaatcatgatcgagccatattagaggcatttggaaaacttt

SEQ ID NO: 2043 - DNA, 60 bp, Homo sapiens
catcatcactgatgctcgtggcagcctgtcgagctccaggagtgatatttaaggccccac

SEQ ID NO: 2044 - DNA, 60 bp, Homo sapiens
atatgaaacattaacaaaactatccgtatcgacactcacacccacacacactcagagtca

SEQ ID NO: 2045 - DNA, 60 bp, Homo sapiens
tttctctcttgtggcccctctggactcctcgatgggaatacttagaattatagtttgaaa

SEQ ID NO: 2046 - DNA, 60 bp, Homo sapiens
tttctctcttgtggcccctctggactcctcgataagagaaacactaaagaactcttggtt

SEQ ID NO: 2047 - DNA, 60 bp, Homo sapiens
agcagagggagccaagtaaaagaccacatcgactatgtcctcaaggacaggagctgtaca

SEQ ID NO: 2048 - DNA, 60 bp, Homo sapiens
tgggaattggtgactggtttcttagagatcgagtgcccttcctggaaggtgtgaacggag

SEQ ID NO: 2049 - DNA, 60 bp, Homo sapiens
tagcttacaccctactttccccagtgcatcgatgatttctattaattgttattatattta

SEQ ID NO: 2050 - DNA, 60 bp, Homo sapiens
tttctctcttgtggcccctctggactcctcgagttatcaaaatattttgagagacagtat

SEQ ID NO: 2051 - DNA, 60 bp, Homo sapiens
ccctcgtccggtaccacgcccctccctctcgatgcaccccaacatataacacaaaactag

SEQ ID NO: 2052 - DNA, 26 bp, Homo sapiens
gagcagccagtcagtaggagaaacca

SEQ ID NO: 2053 - DNA, 26 bp, Homo sapiens
tttgctcttcccattccacctcagaa

SEQ ID NO: 2054 - DNA, 23 bp, Homo sapiens
gtcgttgtctgtctccccactgg

SEQ ID NO: 2055 - DNA, 26 bp, Homo sapiens
gtcttgccattgtttgcttcctttca

SEQ ID NO: 2056 - DNA, 24 bp, Homo sapiens
gagggcttttgtgtcgcttcactc

SEQ ID NO: 2057 - DNA, 24 bp, Homo sapiens
gtctgcacggcatcgtgcacactg

SEQ ID NO: 2058 - DNA, 22 bp, Homo sapiens
gggcgctgcagaacagagcccg

SEQ ID NO: 2059 - DNA, 22 bp, Homo sapiens
acaaagaagtcaaaataattgt

SEQ ID NO: 2060 - DNA, 23 bp, Homo sapiens
gaccaaacgactcacagccaggc

SEQ ID NO: 2061 - DNA, 23 bp, Homo sapiens
cttggcagtggagacatacagcg

SEQ ID NO: 2062 - DNA, 23 bp, Homo sapiens
gtctggtctgaagaggctggagg

SEQ ID NO: 2063 - DNA, 23 bp, Homo sapiens
caagccagtgccagaagacaagc

SEQ ID NO: 2064 - DNA, 24 bp, Homo sapiens
gatctcaaaaaagcactattccat

SEQ ID NO: 2065 - DNA, 24 bp, Homo sapiens
ccccgggcctcggcgccaggagcg

SEQ ID NO: 2066 - DNA, 22 bp, Homo sapiens
gccacacagctcgtgtgggatt

SEQ ID NO: 2067 - DNA, 22 bp, Homo sapiens
ttcacttagcctgcatgagcct

SEQ ID NO: 2068 - DNA, 23 bp, Homo sapiens
tcacaataatggagtactaaagc

SEQ ID NO: 2069 - DNA, 23 bp, Homo sapiens
ctttgaggaagtcaactactgag

SEQ ID NO: 2070 - DNA, 23 bp, Homo sapiens
tggagcagaacagggaggtcaga

SEQ ID NO: 2071 - DNA, 23 bp, Homo sapiens
atagtcggcactgctgagcctgg

SEQ ID NO: 2072 - DNA, 23 bp, Homo sapiens
tggaagaggagtgggcagtggag

SEQ ID NO: 2073 - DNA, 23 bp, Homo sapiens
gcaccctgtccttttctgtcctc

SEQ ID NO: 2074 - DNA, 23 bp, Homo sapiens
tgaagcacaatcgggaccgtgtc

SEQ ID NO: 2075 - DNA, 23 bp, Homo sapiens
ccctcattgacccacctttgccg

SEQ ID NO: 2076 - DNA, 23 bp, Homo sapiens
ttgggagccttctgggagagagg

SEQ ID NO: 2077 - DNA, 23 bp, Homo sapiens
ctgtgaccagtgccaacccattg

SEQ ID NO: 2078 - DNA, 21 bp, Homo sapiens
ccccatctcagttgatggcga

SEQ ID NO: 2079 - DNA, 21 bp, Homo sapiens
tggctaatttttgtattttta

SEQ ID NO: 2080 - DNA, 24 bp, Homo sapiens
gagacggggtttcaccatgttggc

SEQ ID NO: 2081 - DNA, 24 bp, Homo sapiens
tcacttaggctgcatgggcctaga

SEQ ID NO: 2082 - DNA, 26 bp, Homo sapiens
atcaagagcactccagagcagcaacc

SEQ ID NO: 2083 - DNA, 26 bp, Homo sapiens
actgtctattgtcttctatgcctggg

SEQ ID NO: 2084 - DNA, 23 bp, Homo sapiens
cgctggagaaggcagatggagaa

SEQ ID NO: 2085 - DNA, 23 bp, Homo sapiens
gcagccccagaaaagcagaggga

SEQ ID NO: 2086 - DNA, 23 bp, Homo sapiens
gcgtaacttctggcttctgtgcc

SEQ ID NO: 2087 - DNA, 23 bp, Homo sapiens
gagaagggcacaacagagggagc

SEQ ID NO: 2088 - DNA, 26 bp, Homo sapiens
aaaaaaatcatgtattgggccacaaa

SEQ ID NO: 2089 - DNA, 26 bp, Homo sapiens
ggaatggtcatgttgctacctagggt

SEQ ID NO: 2090 - DNA, 26 bp, Homo sapiens
gagagagggagggagggagagggaga

SEQ ID NO: 2091 - DNA, 26 bp, Homo sapiens
gaggccaggctcagggctgtaatccc

SEQ ID NO: 2092 - DNA, 23 bp, Homo sapiens
ggtaggtttcactgtggcgtggt

SEQ ID NO: 2093 - DNA, 23 bp, Homo sapiens
ggcaaacctcggtgttccttggc

SEQ ID NO: 2094 - DNA, 21 bp, Homo sapiens
cactgcagccttgaactcctg

SEQ ID NO: 2095 - DNA, 21 bp, Homo sapiens
agatgggtggatggaggggtg

SEQ ID NO: 2096 - DNA, 22 bp, Homo sapiens
aagaacttaacaaagactggcc

SEQ ID NO: 2097 - DNA, 22 bp, Homo sapiens
attagatatggcggctcaggtc

SEQ ID NO: 2098 - DNA, 23 bp, Homo sapiens
gcagacaagtccagaaagcccga

SEQ ID NO: 2099 - DNA, 23 bp, Homo sapiens
tgggtgaggctctgcttccacac

SEQ ID NO: 2100 - DNA, 26 bp, Homo sapiens
ttatgtgtctcttcctaagggagtcc

SEQ ID NO: 2101 - DNA, 26 bp, Homo sapiens
gactcacaagactgtccccaaagtgg

SEQ ID NO: 2102 - DNA, 23 bp, Homo sapiens
ttgggatgggaactggaggctgg

SEQ ID NO: 2103 - DNA, 23 bp, Homo sapiens
taggtgaatggagaggtggaggg

SEQ ID NO: 2104 - DNA, 23 bp, Homo sapiens
cagaggtggtgctggaggcagat

SEQ ID NO: 2105 - DNA, 23 bp, Homo sapiens
ggagacacatcctgctgccttgt

SEQ ID NO: 2106 - DNA, 22 bp, Homo sapiens
agaattgcttgaacccaggagg

SEQ ID NO: 2107 - DNA, 22 bp, Homo sapiens
gctcaagcaatcctcccacctc

SEQ ID NO: 2108 - DNA, 23 bp, Homo sapiens
ggtggtgttatctgtccttgggc

SEQ ID NO: 2109 - DNA, 23 bp, Homo sapiens
gaatcaggctcccttccctccga

SEQ ID NO: 2110 - DNA, 21 bp, Homo sapiens
tcccggcccactgcaacctcc

SEQ ID NO: 2111 - DNA, 21 bp, Homo sapiens
ctcctctgtccccagtgttac

SEQ ID NO: 2112 - DNA, 23 bp, Homo sapiens
gtcccaccgccctactttggaaa

SEQ ID NO: 2113 - DNA, 23 bp, Homo sapiens
ttccccacagagtgcctgagcca

SEQ ID NO: 2114 - DNA, 23 bp, Homo sapiens
ccactgtcccgctctttccacac

SEQ ID NO: 2115 - DNA, 23 bp, Homo sapiens
ccagaacaaggcacaaagcaggc

SEQ ID NO: 2116 - DNA, 26 bp, Homo sapiens
agtgaggaagtgacacccaagattgc

SEQ ID NO: 2117 - DNA, 26 bp, Homo sapiens
ccctcaaggaagtagcaaaagccaga

SEQ ID NO: 2118 - DNA, 26 bp, Homo sapiens
gctctcgttttgctcgctccttacca

SEQ ID NO: 2119 - DNA, 25 bp, Homo sapiens
tatgaaaataagcccacgcactggc

SEQ ID NO: 2120 - DNA, 26 bp, Homo sapiens
gggaaggaaagaaaacagtgagggtt

SEQ ID NO: 2121 - DNA, 26 bp, Homo sapiens
atgagtgtctggagggagtcctttgg

SEQ ID NO: 2122 - DNA, 26 bp, Homo sapiens
gcagtgtgagggaaatgtgtagtaag

SEQ ID NO: 2123 - DNA, 26 bp, Homo sapiens
tcagccccgtttgtttctcccacaga

SEQ ID NO: 2124 - DNA, 23 bp, Homo sapiens
gtggcaagggctggagaaacaac

SEQ ID NO: 2125 - DNA, 23 bp, Homo sapiens
ccactgtcccgctctttccacac

SEQ ID NO: 2126 - DNA, 21 bp, Homo sapiens
tgtctccatagggagcagaag

SEQ ID NO: 2127 - DNA, 21 bp, Homo sapiens
cccacctctgactcggatagt

SEQ ID NO: 2128 - DNA, 26 bp, Homo sapiens
acaccccttgatgttactttgtcaca

SEQ ID NO: 2129 - DNA, 26 bp, Homo sapiens
ctttcgtttcccttctccaagtcctt

SEQ ID NO: 2130 - DNA, 25 bp, Homo sapiens
caatccctttgtgacttctgaccga

SEQ ID NO: 2131 - DNA, 26 bp, Homo sapiens
attttctatttcttcctcccctcgta

SEQ ID NO: 2132 - DNA, 25 bp, Homo sapiens
atctaagaggctgggatgaggttta

SEQ ID NO: 2133 - DNA, 25 bp, Homo sapiens
aatcctgtcccttggcactgctctg

SEQ ID NO: 2134 - DNA, 26 bp, Homo sapiens
gctaaaatgagacagacagaatgggc

SEQ ID NO: 2135 - DNA, 26 bp, Homo sapiens
gaagttgtccatctgggtgaggttgc

SEQ ID NO: 2136 - DNA, 23 bp, Homo sapiens
aagcaaaactgtagaaaatttgg

SEQ ID NO: 2137 - DNA, 23 bp, Homo sapiens
tgcctgaggttaatgattaccct

SEQ ID NO: 2138 - DNA, 26 bp, Homo sapiens
aacttgctgtcattccaccactgtcc

SEQ ID NO: 2139 - DNA, 26 bp, Homo sapiens
gttgtagtgaatggtttctcttcttc

SEQ ID NO: 2140 - DNA, 26 bp, Homo sapiens
cgctctttccacactcacatccctgt

SEQ ID NO: 2141 - DNA, 25 bp, Homo sapiens
taaacacttgccgaaatgagagcgg

SEQ ID NO: 2142 - DNA, 23 bp, Homo sapiens
cccaacccttgtccctgtgaagc

SEQ ID NO: 2143 - DNA, 23 bp, Homo sapiens
gccaagagcctgagggacaatag

SEQ ID NO: 2144 - DNA, 24 bp, Homo sapiens
catgatcttcatacctggcatctg

SEQ ID NO: 2145 - DNA, 24 bp, Homo sapiens
agtatgattggcctgggagagtgc

SEQ ID NO: 2146 - DNA, 26 bp, Homo sapiens
agtgaggagctagtggccaggagggg

SEQ ID NO: 2147 - DNA, 26 bp, Homo sapiens
ctcccacccaggatattaggaaaaaa

SEQ ID NO: 2148 - DNA, 26 bp, Homo sapiens
cgctctttccacactcacatccctgt

SEQ ID NO: 2149 - DNA, 26 bp, Homo sapiens
ctatccagtgtgttcataagaagccc

SEQ ID NO: 2150 - DNA, 23 bp, Homo sapiens
cccaccttatccatcctacacgc

SEQ ID NO: 2151 - DNA, 23 bp, Homo sapiens
gccacaggtcccctaagtgctac

SEQ ID NO: 2152 - DNA, 28 bp, Homo sapiens
gagcagccagtcagtaggagaaaccaaa

SEQ ID NO: 2153 - DNA, 24 bp, Homo sapiens
cccacccacccctcgccctgctcc

SEQ ID NO: 2154 - DNA, 23 bp, Homo sapiens
ctgtgtttagcccataaacttca

SEQ ID NO: 2155 - DNA, 22 bp, Homo sapiens
agcggatgtctccaggaggagg

SEQ ID NO: 2156 - DNA, 26 bp, Homo sapiens
ggctggactgtctcagggacctactc

SEQ ID NO: 2157 - DNA, 28 bp, Homo sapiens
taggtctggtctgaagaggctggaggat

SEQ ID NO: 2158 - DNA, 21 bp, Homo sapiens
taagaactggtttagctcagg

SEQ ID NO: 2159 - DNA, 25 bp, Homo sapiens
cctccatgatctgggttgctttcaa

SEQ ID NO: 2160 - DNA, 21 bp, Homo sapiens
gagacaataactgtcttaaaa

SEQ ID NO: 2161 - DNA, 28 bp, Homo sapiens
ggctggagcagaacagggaggtcagagc

SEQ ID NO: 2162 - DNA, 24 bp, Homo sapiens
ccgacacaggcgggcagccactcc

SEQ ID NO: 2163 - DNA, 28 bp, Homo sapiens
aatggtgaagcacaatcgggaccgtgtc

SEQ ID NO: 2164 - DNA, 25 bp, Homo sapiens
gtgctgcccagacaggaccccttgc

SEQ ID NO: 2165 - DNA, 23 bp, Homo sapiens
gtcagagaccaaggagtcattcc

SEQ ID NO: 2166 - DNA, 22 bp, Homo sapiens
ggccaggctggttttgaactcc

SEQ ID NO: 2167 - DNA, 25 bp, Homo sapiens
ggcagagcacgcaggaaacggctgg

SEQ ID NO: 2168 - DNA, 28 bp, Homo sapiens
gtatccgctggagaaggcagatggagaa

SEQ ID NO: 2169 - DNA, 28 bp, Homo sapiens
ggactgcgtaacttctggcttctgtgcc

SEQ ID NO: 2170 - DNA, 23 bp, Homo sapiens
aagtccatgaaatattcgcaaaa

SEQ ID NO: 2171 - DNA, 23 bp, Homo sapiens
aaggaaggagagagggagggagg

SEQ ID NO: 2172 - DNA, 23 bp, Homo sapiens
cacctgcccctccacctgctgac

SEQ ID NO: 2173 - DNA, 23 bp, Homo sapiens
tgcctcagtcgcctgagtagcta

SEQ ID NO: 2174 - DNA, 26 bp, Homo sapiens
cttaacaaagactggccaccaccatg

SEQ ID NO: 2175 - DNA, 28 bp, Homo sapiens
cagacaagtccagaaagcccgaagttgg

SEQ ID NO: 2176 - DNA, 28 bp, Homo sapiens
ggactgaggctttcttgctcttctgtgc

SEQ ID NO: 2177 - DNA, 28 bp, Homo sapiens
gaggttgggatgggaactggaggctggg

SEQ ID NO: 2178 - DNA, 28 bp, Homo sapiens
gcttggcagaggtggtgctggaggcaga

SEQ ID NO: 2179 - DNA, 22 bp, Homo sapiens
gccggtaatcccagctacttgg

SEQ ID NO: 2180 - DNA, 28 bp, Homo sapiens
ctctgaggtaggcttggcttcggtggtg

SEQ ID NO: 2181 - DNA, 22 bp, Homo sapiens
tttttttttgagagagagagtc

SEQ ID NO: 2182 - DNA, 23 bp, Homo sapiens
cagccaatcaggtcccaccgccc

SEQ ID NO: 2183 - DNA, 25 bp, Homo sapiens
cgctggacatccccaccgcacctcc

SEQ ID NO: 2184 - DNA, 28 bp, Homo sapiens
ccagtgaggaagtgacacccaagattgc

SEQ ID NO: 2185 - DNA, 28 bp, Homo sapiens
gctctcgttttgctcgctccttaccaac

SEQ ID NO: 2186 - DNA, 28 bp, Homo sapiens
gggatgaggagtaagagacacgggacag

SEQ ID NO: 2187 - DNA, 28 bp, Homo sapiens
gctaaagcagtgtgagggaaatgtgtag

SEQ ID NO: 2188 - DNA, 28 bp, Homo sapiens
ggagcctacctcttccttctcactgacc

SEQ ID NO: 2189 - DNA, 24 bp, Homo sapiens
aaactacagcctgtgggccagtcc

SEQ ID NO: 2190 - DNA, 25 bp, Homo sapiens
ggtatggcaagggaggtgagggttg

SEQ ID NO: 2191 - DNA, 27 bp, Homo sapiens
atctgcctctgcttttctcttccccta

SEQ ID NO: 2192 - DNA, 28 bp, Homo sapiens
gtatttggcagggagtaggtggagggta

SEQ ID NO: 2193 - DNA, 28 bp, Homo sapiens
gctgtgaagaggcaagaatgggtatggg

SEQ ID NO: 2194 - DNA, 24 bp, Homo sapiens
aatagttgaaaatttgtgatattc

SEQ ID NO: 2195 - DNA, 25 bp, Homo sapiens
cgctggacatccccaccgcacctcc

SEQ ID NO: 2196 - DNA, 26 bp, Homo sapiens
ccgctctttccacactcacatccctg

SEQ ID NO: 2197 - DNA, 24 bp, Homo sapiens
ccccaacccttgtccctgtgaagc

SEQ ID NO: 2198 - DNA, 25 bp, Homo sapiens
aagtctgcacatttaactgtcaact

SEQ ID NO: 2199 - DNA, 26 bp, Homo sapiens
gagctagtggccaggaggggctcatg

SEQ ID NO: 2200 - DNA, 25 bp, Homo sapiens
cgctggacatccccaccgcacctcc

SEQ ID NO: 2201 - DNA, 27 bp, Homo sapiens
gctgagcacttcctccagggcatcccc

SEQ ID NO: 2202 - DNA, 28 bp, Homo sapiens
caagtttgctcttcccattccacctcag

SEQ ID NO: 2203 - DNA, 36 bp, Homo sapiens
acgctcaaacctcgacttagaactgtaattgaattt

SEQ ID NO: 2204 - DNA, 28 bp, Homo sapiens
ggcaaaaccccgtctctactaaacacaa

SEQ ID NO: 2205 - DNA, 37 bp, Homo sapiens
agcaacttgtctatcttgttcgactcactgcaacccc

SEQ ID NO: 2206 - DNA, 23 bp, Homo sapiens
tcagccgtgtccccggggccctc

SEQ ID NO: 2207 - DNA, 40 bp, Homo sapiens
acccactggcactggtgatcgactgcgccccgctgctgtt

SEQ ID NO: 2208 - DNA, 22 bp, Homo sapiens
aaaactgtctaacaaagaagtc

SEQ ID NO: 2209 - DNA, 40 bp, Homo sapiens
ataaagaaggatttagtgtcgaactcctgacttcaggtga

SEQ ID NO: 2210 - DNA, 28 bp, Homo sapiens
cgggtgaatggattatgatgggacaagc

SEQ ID NO: 2211 - DNA, 32 bp, Homo sapiens
aatttggttcctcccttcgaccctgtgcggac

SEQ ID NO: 2212 - DNA, 27 bp, Homo sapiens
ggttctgtatggtggatgggcaagggc

SEQ ID NO: 2213 - DNA, 36 bp, Homo sapiens
acctgctccaagtctttccatctcgaagtatcctcc

SEQ ID NO: 2214 - DNA, 21 bp, Homo sapiens
gcctcggcgccaggagcgggg

SEQ ID NO: 2215 - DNA, 40 bp, Homo sapiens
ggaagctgctctaaggtttcgaatccctagcagccagtcc

SEQ ID NO: 2216 - DNA, 25 bp, Homo sapiens
ctcacttaggctgcatgggcctaga

SEQ ID NO: 2217 - DNA, 40 bp, Homo sapiens
tatttggttctttaaacctcgaggctcatgcaggctaact

SEQ ID NO: 2218 - DNA, 21 bp, Homo sapiens
acctgaactggggtgtgagag

SEQ ID NO: 2219 - DNA, 40 bp, Homo sapiens
tctgaataatctttttgctcgagagggtttggcccccagc

SEQ ID NO: 2220 - DNA, 23 bp, Homo sapiens
ccttctgtgcccacgcctggtgc

SEQ ID NO: 2221 - DNA, 29 bp, Homo sapiens
aggagggtcgagctccactcagctgggtt

SEQ ID NO: 2222 - DNA, 28 bp, Homo sapiens
gggaccaagggttttcagtcttagcacc

SEQ ID NO: 2223 - DNA, 34 bp, Homo sapiens
tcagcctgcattgtcgaactcctaagctcaggtg

SEQ ID NO: 2224 - DNA, 26 bp, Homo sapiens
cgtatcccctggtgggctcctgaggc

SEQ ID NO: 2225 - DNA, 35 bp, Homo sapiens
tgtggccggctcgatttcaagaagttggtgaaagc

SEQ ID NO: 2226 - DNA, 28 bp, Homo sapiens
ctgtgaccagtgccaacccattgtaacc

SEQ ID NO: 2227 - DNA, 32 bp, Homo sapiens
aaccaccccagggctcgacaaggtcaaatgca

SEQ ID NO: 2228 - DNA, 23 bp, Homo sapiens
aggtgcataccaccacacctggc

SEQ ID NO: 2229 - DNA, 40 bp, Homo sapiens
ggaattattgcagtagtctcgattactttttcgtcttggg

SEQ ID NO: 2230 - DNA, 22 bp, Homo sapiens
ctcacttaggctgcatgggcct

SEQ ID NO: 2231 - DNA, 40 bp, Homo sapiens
tagttttaatggaatatgtcgaggctcatgcaggctaact

SEQ ID NO: 2232 - DNA, 28 bp, Homo sapiens
cccagtcatcaagtatggacacaaggac

SEQ ID NO: 2233 - DNA, 35 bp, Homo sapiens
tctacaattcgagaagacagtccagagctgaaccc

SEQ ID NO: 2234 - DNA, 27 bp, Homo sapiens
gccaggcagccccagaaaagcagaggg

SEQ ID NO: 2235 - DNA, 32 bp, Homo sapiens
ttttcttcgagccctaccaacccagacagctc

SEQ ID NO: 2236 - DNA, 27 bp, Homo sapiens
caggacaagactgggatttgccacccc

SEQ ID NO: 2237 - DNA, 35 bp, Homo sapiens
tcatatcatcgacaggcacctttccaggctcttcc

SEQ ID NO: 2238 - DNA, 23 bp, Homo sapiens
ctacctagggttagggtttcctt

SEQ ID NO: 2239 - DNA, 40 bp, Homo sapiens
aatacagtgagacccatctcgaagtctgaggtaaaacaca

SEQ ID NO: 2240 - DNA, 23 bp, Homo sapiens
actttgggaggccgtggtgggaa

SEQ ID NO: 2241 - DNA, 40 bp, Homo sapiens
gaggagggagaagggaggtcgaactcccgacctcaggtga

SEQ ID NO: 2242 - DNA, 28 bp, Homo sapiens
ggtggctcctggcaaacctcggtgttcc

SEQ ID NO: 2243 - DNA, 35 bp, Homo sapiens
agatgatcgaacttctgacctcaggtgatctgccc

SEQ ID NO: 2244 - DNA, 23 bp, Homo sapiens
ggaggggtggaggggtggataga

SEQ ID NO: 2245 - DNA, 40 bp, Homo sapiens
ggctggttgtcttttttttcgatccacccatccacccatc

SEQ ID NO: 2246 - DNA, 26 bp, Homo sapiens
gattagatatggcggctcaggtcatg

SEQ ID NO: 2247 - DNA, 40 bp, Homo sapiens
acttggcacatggaggcctcgacacttcctcagacgctct

SEQ ID NO: 2248 - DNA, 27 bp, Homo sapiens
ccccttggtccttgggtgaggctctgc

SEQ ID NO: 2249 - DNA, 35 bp, Homo sapiens
agggcactgaattcgatctcagactccatcagagc

SEQ ID NO: 2250 - DNA, 28 bp, Homo sapiens
gagactcacaagactgtccccaaagtgg

SEQ ID NO: 2251 - DNA, 36 bp, Homo sapiens
tcctccctactgcctcccaggctcaaaatcgatata

SEQ ID NO: 2252 - DNA, 24 bp, Homo sapiens
gagggtggacagacagggaggtgg

SEQ ID NO: 2253 - DNA, 32 bp, Homo sapiens
agcgaaggagaaagtcgatccacccatccacc

SEQ ID NO: 2254 - DNA, 25 bp, Homo sapiens
gcctctcccttcccacctacttggc

SEQ ID NO: 2255 - DNA, 34 bp, Homo sapiens
tgtgactcgatcccttactcctgaggcaactgtg

SEQ ID NO: 2256 - DNA, 22 bp, Homo sapiens
ccttgacctccctggctcaagc

SEQ ID NO: 2257 - DNA, 40 bp, Homo sapiens
actatgaggatataccattcgagaccagcctgagcaacat

SEQ ID NO: 2258 - DNA, 27 bp, Homo sapiens
gggtctctgcgggttcttggatgcgag

SEQ ID NO: 2259 - DNA, 28 bp, Homo sapiens
ttttagcccctcgagcgacggaggcgac

SEQ ID NO: 2260 - DNA, 22 bp, Homo sapiens
tctgtccccagtgttaccacag

SEQ ID NO: 2261 - DNA, 40 bp, Homo sapiens
tcgcttgagggcaggagttcgagatcggacacctccaact

SEQ ID NO: 2262 - DNA, 28 bp, Homo sapiens
ccttccccacagagtgcctgagccacac

SEQ ID NO: 2263 - DNA, 32 bp, Homo sapiens
atcgcaagttcaggagttcgagtccccaggtg

SEQ ID NO: 2264 - DNA, 25 bp, Homo sapiens
gcccagaacaaggcacaaagcaggc

SEQ ID NO: 2265 - DNA, 32 bp, Homo sapiens
tcttgtggcccctctggactcctcgaccatat

SEQ ID NO: 2266 - DNA, 28 bp, Homo sapiens
gccctcaaggaagtagcaaaagccagaa

SEQ ID NO: 2267 - DNA, 33 bp, Homo sapiens
tctgtgcagaagtcctcgaactcctgacctcag

SEQ ID NO: 2268 - DNA, 28 bp, Homo sapiens
cagtcctaaaggtatcctctctgccagg

SEQ ID NO: 2269 - DNA, 38 bp, Homo sapiens
tgattatttgcccactcccttccacttcaatcgaagat

SEQ ID NO: 2270 - DNA, 25 bp, Homo sapiens
gcagtgggctgataccgtctctccg

SEQ ID NO: 2271 - DNA, 32 bp, Homo sapiens
agacaaggtctcgactctccacgcatctctgc

SEQ ID NO: 2272 - DNA, 25 bp, Homo sapiens
gccgtcagccccgtttgtttctccc

SEQ ID NO: 2273 - DNA, 39 bp, Homo sapiens
tcaataaaatcgaatcttcgcggactccacgcatctgtg

SEQ ID NO: 2274 - DNA, 28 bp, Homo sapiens
ccccaccgcacctcctcacgaatcctga

SEQ ID NO: 2275 - DNA, 34 bp, Homo sapiens
acttgggtcatctttcttcgaggagtccagaggg

SEQ ID NO: 2276 - DNA, 24 bp, Homo sapiens
tcggatagtgctgggattacaggc

SEQ ID NO: 2277 - DNA, 40 bp, Homo sapiens
aaaattcaaattttagtgtcgaactcctaacctcaggtga

SEQ ID NO: 2278 - DNA, 28 bp, Homo sapiens
ttctttcgtttcccttctccaagtcctt

SEQ ID NO: 2279 - DNA, 33 bp, Homo sapiens
acttggactaccctctccgtccacctcgaattc

SEQ ID NO: 2280 - DNA, 26 bp, Homo sapiens
gctctcgttttgctcgctccttacca

SEQ ID NO: 2281 - DNA, 35 bp, Homo sapiens
agtacaatgttcgattgaagtggaagggagtgggc

SEQ ID NO: 2282 - DNA, 25 bp, Homo sapiens
cctgtcccttggcactgctctggag

SEQ ID NO: 2283 - DNA, 36 bp, Homo sapiens
tcatgatcgagccatattagaggcatttggaaaact

SEQ ID NO: 2284 - DNA, 28 bp, Homo sapiens
gagaagttgtccatctgggtgaggttgc

SEQ ID NO: 2285 - DNA, 34 bp, Homo sapiens
agcctgtcgagctccaggagtgatatttaaggcc

SEQ ID NO: 2286 - DNA, 24 bp, Homo sapiens
cctgctgcttgcctgaggttaatg

SEQ ID NO: 2287 - DNA, 40 bp, Homo sapiens
ttaacaaaactatccgtatcgacactcacacccacacaca

SEQ ID NO: 2288 - DNA, 28 bp, Homo sapiens
gctatccctactcccctatctccaaacc

SEQ ID NO: 2289 - DNA, 32 bp, Homo sapiens
ttgtggcccctctggactcctcgatgggaata

SEQ ID NO: 2290 - DNA, 25 bp, Homo sapiens
taaacacttgccgaaatgagagcgg

SEQ ID NO: 2291 - DNA, 32 bp, Homo sapiens
tctcttgtggcccctctggactcctcgataag

SEQ ID NO: 2292 - DNA, 28 bp, Homo sapiens
gccaagagcctgagggacaatagaatgg

SEQ ID NO: 2293 - DNA, 34 bp, Homo sapiens
agaccacatcgactatgtcctcaaggacaggagc

SEQ ID NO: 2294 - DNA, 25 bp, Homo sapiens
atgattggcctgggagagtgccatc

SEQ ID NO: 2295 - DNA, 40 bp, Homo sapiens
tgactggtttcttagagatcgagtgcccttcctggaaggt

SEQ ID NO: 2296 - DNA, 26 bp, Homo sapiens
atgaccgaaggggtgtatacccactg

SEQ ID NO: 2297 - DNA, 40 bp, Homo sapiens
cctactttccccagtgcatcgatgatttctattaattgtt

SEQ ID NO: 2298 - DNA, 28 bp, Homo sapiens
gggcaatggattagggacactgaagcag

SEQ ID NO: 2299 - DNA, 32 bp, Homo sapiens
tctcttgtggcccctctggactcctcgagtta

SEQ ID NO: 2300 - DNA, 28 bp, Homo sapiens
ggtcccctaagtgctacaagtgtttgcg

SEQ ID NO: 2301 - DNA, 27 bp, Homo sapiens
taccacgcccctccctctcgatgcacc

SEQ ID NO: 2302 - DNA, 35 bp, Homo sapiens
tcaattacagttctaagtcgaggtttgagcgtggg

SEQ ID NO: 2303 - DNA, 36 bp, Homo sapiens
tgcagtgagtcgaacaagatagacaagttgctcatc

SEQ ID NO: 2304 - DNA, 40 bp, Homo sapiens
aacagcagcggggcgcagtcgatcaccagtgccagtgggt

SEQ ID NO: 2305 - DNA, 40 bp, Homo sapiens
tcacctgaagtcaggagttcgacactaaatccttctttat

SEQ ID NO: 2306 - DNA, 32 bp, Homo sapiens
tgtccgcacagggtcgaagggaggaaccaaat

SEQ ID NO: 2307 - DNA, 36 bp, Homo sapiens
aggaggatacttcgagatggaaagacttggagcagg

SEQ ID NO: 2308 - DNA, 40 bp, Homo sapiens
ggactggctgctagggattcgaaaccttagagcagcttcc

SEQ ID NO: 2309 - DNA, 40 bp, Homo sapiens
agttagcctgcatgagcctcgaggtttaaagaaccaaata

SEQ ID NO: 2310 - DNA, 40 bp, Homo sapiens
gctgggggccaaaccctctcgagcaaaaagattattcaga

SEQ ID NO: 2311 - DNA, 29 bp, Homo sapiens
aacccagctgagtggagctcgaccctcct

SEQ ID NO: 2312 - DNA, 34 bp, Homo sapiens
tcacctgagcttaggagttcgacaatgcaggctg

SEQ ID NO: 2313 - DNA, 32 bp, Homo sapiens
accaacttcttgaaatcgagccggccacaggg

SEQ ID NO: 2314 - DNA, 37 bp, Homo sapiens
acataatactgtgcatttgaccttgtcgagccctggg

SEQ ID NO: 2315 - DNA, 40 bp, Homo sapiens
cccaagacgaaaaagtaatcgagactactgcaataattcc

SEQ ID NO: 2316 - DNA, 40 bp, Homo sapiens
agttagcctgcatgagcctcgacatattccattaaaacta

SEQ ID NO: 2317 - DNA, 34 bp, Homo sapiens
tcagctctggactgtcttctcgaattgtagaggg

SEQ ID NO: 2318 - DNA, 31 bp, Homo sapiens
agctgtctgggttggtagggctcgaagaaaa

SEQ ID NO: 2319 - DNA, 35 bp, Homo sapiens
tggaagagcctggaaaggtgcctgtcgatgatatg

SEQ ID NO: 2320 - DNA, 40 bp, Homo sapiens
tgtgttttacctcagacttcgagatgggtctcactgtatt

SEQ ID NO: 2321 - DNA, 40 bp, Homo sapiens
tcacctgaggtcgggagttcgacctcccttctccctcctc

SEQ ID NO: 2322 - DNA, 36 bp, Homo sapiens
agatcacctgaggtcagaagttcgatcatctcctgc

SEQ ID NO: 2323 - DNA, 40 bp, Homo sapiens
gatgggtggatgggtggatcgaaaaaaaagacaaccagcc

SEQ ID NO: 2324 - DNA, 40 bp, Homo sapiens
agagcgtctgaggaagtgtcgaggcctccatgtgccaagt

SEQ ID NO: 2325 - DNA, 35 bp, Homo sapiens
tgctctgatggagtctgagatcgaattcagtgccc

SEQ ID NO: 2326 - DNA, 35 bp, Homo sapiens
tatatcgattttgagcctgggaggcagtagggagg

SEQ ID NO: 2327 - DNA, 32 bp, Homo sapiens
tgggtggatgggtggatcgactttctccttcg

SEQ ID NO: 2328 - DNA, 35 bp, Homo sapiens
acagttgcctcaggagtaagggatcgagtcacaag

SEQ ID NO: 2329 - DNA, 40 bp, Homo sapiens
atgttgctcaggctggtctcgaatggtatatcctcatagt

SEQ ID NO: 2330 - DNA, 28 bp, Homo sapiens
gtcgcctccgtcgctcgaggggctaaaa

SEQ ID NO: 2331 - DNA, 40 bp, Homo sapiens
agttggaggtgtccgatctcgaactcctgccctcaagcga

SEQ ID NO: 2332 - DNA, 32 bp, Homo sapiens
cacctggggactcgaactcctgaacttgcgat

SEQ ID NO: 2333 - DNA, 33 bp, Homo sapiens
tccactcactgatatggtcgaggagtccagagg

SEQ ID NO: 2334 - DNA, 32 bp, Homo sapiens
acctgaggtcaggagttcgaggacttctgcac

SEQ ID NO: 2335 - DNA, 37 bp, Homo sapiens
atcttcgattgaagtggaagggagtgggcaaataatc

SEQ ID NO: 2336 - DNA, 32 bp, Homo sapiens
tggcagagatgcgtggagagtcgagaccttgt

SEQ ID NO: 2337 - DNA, 39 bp, Homo sapiens
acacagatgcgtggagtccgcgaagattcgattttattg

SEQ ID NO: 2338 - DNA, 32 bp, Homo sapiens
tggcccctctggactcctcgaagaaagatgac

SEQ ID NO: 2339 - DNA, 40 bp, Homo sapiens
tcacctgaggttaggagttcgacactaaaatttgaatttt

SEQ ID NO: 2340 - DNA, 33 bp, Homo sapiens
agaattcgaggtggacggagagggtagtccaag

SEQ ID NO: 2341 - DNA, 35 bp, Homo sapiens
tgcccactcccttccacttcaatcgaacattgtac

SEQ ID NO: 2342 - DNA, 34 bp, Homo sapiens
ttttccaaatgcctctaatatggctcgatcatga

SEQ ID NO: 2343 - DNA, 32 bp, Homo sapiens
aatatcactcctggagctcgacaggctgccac

SEQ ID NO: 2344 - DNA, 40 bp, Homo sapiens
tgtgtgtgggtgtgagtgtcgatacggatagttttgttaa

SEQ ID NO: 2345 - DNA, 32 bp, Homo sapiens
ttctaagtattcccatcgaggagtccagaggg

SEQ ID NO: 2346 - DNA, 38 bp, Homo sapiens
agttctttagtgtttctcttatcgaggagtccagaggg

SEQ ID NO: 2347 - DNA, 34 bp, Homo sapiens
agctcctgtccttgaggacatagtcgatgtggtc

SEQ ID NO: 2348 - DNA, 40 bp, Homo sapiens
accttccaggaagggcactcgatctctaagaaaccagtca

SEQ ID NO: 2349 - DNA, 40 bp, Homo sapiens
aacaattaatagaaatcatcgatgcactggggaaagtagg

SEQ ID NO: 2350 - DNA, 31 bp, Homo sapiens
aatattttgataactcgaggagtccagaggg

SEQ ID NO: 2351 - DNA, 27 bp, Homo sapiens
ggtgcatcgagagggaggggcgtggta

SEQ ID NO: 2352 - DNA, 60 bp, Homo sapiens
catacatttattactgatcttttgatcatcgacctcctagccctgtctcatccaagaggg

SEQ ID NO: 2353 - DNA, 60 bp, Homo sapiens
taccagcacaggccccgttcctctctgttcgattacccctgtaatgccagtgtggtaatg

SEQ ID NO: 2354 - DNA, 60 bp, Homo sapiens
tatgttctaatcctgggagtcttctcggtcgagaccagcatggccaacaagaagaaatcc

SEQ ID NO: 2355 - DNA, 60 bp, Homo sapiens
gaaccatacacttgacaccaccaccacctcgagaaggtccagattgctgccatagttctg

SEQ ID NO: 2356 - DNA, 60 bp, Homo sapiens
tgacacaaagatattctgctttgccagctcgaaggaatattctagcctttgattcctttc

SEQ ID NO: 2357 - DNA, 60 bp, Homo sapiens
cgctactctcattggtcatacctgttgttcgaattctgaataccgtcccctaaaataatg

SEQ ID NO: 2358 - DNA, 60 bp, Homo sapiens
ctgcactccaacagagggagactctgtctcgatgccctgcctgtgcctgaggtgattagg

SEQ ID NO: 2359 - DNA, 60 bp, Homo sapiens
aggcttatacttttattgtatttctaaatcgaagcagggaagggagaaaaggccttctga

SEQ ID NO: 2360 - DNA, 60 bp, Homo sapiens
gggtttcaccatgttggtcaggctggtctcgagatcagcctggccaacgttgtgaaaccc

SEQ ID NO: 2361 - DNA, 60 bp, Homo sapiens
gtaagaccttatctctaaaagaaatgtttcgactctccacgcatctctgccacccgagtg

SEQ ID NO: 2362 - DNA, 60 bp, Homo sapiens
tttctctcttgtggcccctctggactcctcgatctgcatcttgactctgccacatacctc

SEQ ID NO: 2363 - DNA, 60 bp, Homo sapiens
gagccaaagcatttcccgctcttaaacctcgagccccttactctccctggagcctttgct

SEQ ID NO: 2364 - DNA, 60 bp, Homo sapiens
acattttttgctcctgcttgttgaggtttcgagacaagcctggccaacatggtgaaaccc

SEQ ID NO: 2365 - DNA, 60 bp, Homo sapiens
aatgcattaacttacacagaatacctactcgacctagggagacaagtcctgcctcagaaa

SEQ ID NO: 2366 - DNA, 60 bp, Homo sapiens
aaggaaggaagcgttgttacccaggagatcgagtggtttgtcatggtaaagaaaaaatgc

SEQ ID NO: 2367 - DNA, 60 bp, Homo sapiens
aaacaacactggggaatgaaaaggttggtcgaagatactttcagattatagcctttgtta

SEQ ID NO: 2368 - DNA, 60 bp, Homo sapiens
tacctttgacaagactcccagaggcacttcgaagacccacttcctacgtcagacgttttc

SEQ ID NO: 2369 - DNA, 60 bp, Homo sapiens
acccaagtctttattagcgaagtcacgttcgatccatccatccattcatccatccaaaca

SEQ ID NO: 2370 - DNA, 60 bp, Homo sapiens
cggtgcagcggcggccgcgggctcagaatcgatccatccatccatcatccacttatctat

SEQ ID NO: 2371 - DNA, 60 bp, Homo sapiens
cgtggccatttgcaccaccggggtgtcctcgaaaccaacctgaccaacatgaagaaacct

SEQ ID NO: 2372 - DNA, 60 bp, Homo sapiens
gtcaaggactgcgccctctcagattctttcgacttatttttttaaacctttttctcttgc

SEQ ID NO: 2373 - DNA, 60 bp, Homo sapiens
gggcccagcctgaccaggaaggctgccttcgacacacccccccaccacccacctcctctc

SEQ ID NO: 2374 - DNA, 60 bp, Homo sapiens
cctgacttcatacttgttgacttctaagtcgagggagccagagggggaatccaaccaaag

SEQ ID NO: 2375 - DNA, 60 bp, Homo sapiens
cacctgcttgggacatgagggcttcccctcgatatcttaacctcgtgatccgcctgcctc

SEQ ID NO: 2376 - DNA, 60 bp, Homo sapiens
ccgggcccacctgcgggtgcaccttaaatcgatccacctgacccctgcctcctccccaac

SEQ ID NO: 2377 - DNA, 60 bp, Homo sapiens
tatcaatttgtctattttattccatttatcgacaggaaggaaggaggaaggggcgctctg

SEQ ID NO: 2378 - DNA, 60 bp, Homo sapiens
attatttgcccactcccttccacttcaatcgattcacacaccattcctcctcagcacacc

SEQ ID NO: 2379 - DNA, 60 bp, Homo sapiens
aaagtaaaacatgtcctttaatggacattcgaacactttacactcagttcctcactgaac

SEQ ID NO: 2380 - DNA, 60 bp, Homo sapiens
gaagagtgacagaaggaagagaagtaaatcgatcttcagcaccctgttcccaggaagtgg

SEQ ID NO: 2381 - DNA, 60 bp, Homo sapiens
aatgaagcatttacttatgagaatattctcgatctgaccttgtgatccacccgcctcagc

SEQ ID NO: 2382 - DNA, 60 bp, Homo sapiens
caggccttctctctgtgctatgaagacttcgaggaggaggttgagcggactccgaagcat

SEQ ID NO: 2383 - DNA, 60 bp, Homo sapiens
tctctgatatctgacagtttttatattttcgaatacctgacctcgtgatccacccgcctc

SEQ ID NO: 2384 - DNA, 60 bp, Homo sapiens
tcccccacctcccttccaaccttccccttcgattcatgtcagtgacttgccatacatatc

SEQ ID NO: 2385 - DNA, 60 bp, Homo sapiens
aggtcttactatgttgcctaggctgatttcgatcacctgctccaccctgactcattccaa

SEQ ID NO: 2386 - DNA, 60 bp, Homo sapiens
accttccgagccaccatttccccactgctcgactccccagtctgacctggcctcacctgg

SEQ ID NO: 2387 - DNA, 60 bp, Homo sapiens
aagcctttttgccaaactacaggcttattcgaccgcccacccctctcccactccccatcc

SEQ ID NO: 2388 - DNA, 60 bp, Homo sapiens
tttctgggtgactgtaattgctagatgttcgaactcctgacctcctgatccccctgcttt

SEQ ID NO: 2389 - DNA, 60 bp, Homo sapiens
acagccacaatcccttcaatgatgaggctcgaccaatttcaaatgtatatatagtcttta

SEQ ID NO: 2390 - DNA, 60 bp, Homo sapiens
gacccctggcttgcaacaggtgcctgaatcgaagatcctccccacccgcccccctcccac

SEQ ID NO: 2391 - DNA, 60 bp, Homo sapiens
ccatcttctcccttgccctctattattttcgatctgtgtaaacaaaagggacacagaacc

SEQ ID NO: 2392 - DNA, 60 bp, Homo sapiens
gaaaaaaggactctgcctacaccttgactcgataaatggtagtgtgaagttggcactagg

SEQ ID NO: 2393 - DNA, 60 bp, Homo sapiens
ggaaaaaaatatttttttctttcacacttcgaccccaccccaccacctccaatccatcag

SEQ ID NO: 2394 - DNA, 60 bp, Homo sapiens
aaacaacactggggaatgaaaaggttggtcgattcagagaaaatcacagattgttaaaat

SEQ ID NO: 2395 - DNA, 60 bp, Homo sapiens
cagcttacaagggtgagttgtgcgtatttcgacctccttccttgcaatgctctctccttc

SEQ ID NO: 2396 - DNA, 60 bp, Homo sapiens
aggtgactattaaactacccaataattctcgaagatcctccccacccgcccccctcccac

SEQ ID NO: 2397 - DNA, 60 bp, Homo sapiens
ttttattcctcacctgactgggagcagctcgagtccagtatttttccaatactagttgtg

SEQ ID NO: 2398 - DNA, 60 bp, Homo sapiens
caccctggctggaagggatggtgtgttctcgaggttgcagagcagagcatgccaaggaag

SEQ ID NO: 2399 - DNA, 60 bp, Homo sapiens
ctaaggaagcaggaggtcttctgaatcctcgatgactgtgagaaacttttgttgcattca

SEQ ID NO: 2400 - DNA, 60 bp, Homo sapiens
tcccccacctcccttccaaccttccccttcgaactgctgagtaggaagcgctctagcagg

SEQ ID NO: 2401 - DNA, 60 bp, Homo sapiens
actaatctccacacggcaaccagacacgtcgagaccatcctggctaacacagtgcaaccc

SEQ ID NO: 2402 - DNA, 24 bp, Homo sapiens
accatgtagcccaggctggtcttg

SEQ ID NO: 2403 - DNA, 24 bp, Homo sapiens
aaataccataatggattcaagtta

SEQ ID NO: 2404 - DNA, 23 bp, Homo sapiens
gggatggctctgggtgaatggca

SEQ ID NO: 2405 - DNA, 24 bp, Homo sapiens
cccacagcacctttctatggttcc

SEQ ID NO: 2406 - DNA, 24 bp, Homo sapiens
acgaaatgatggggtggtggggtg

SEQ ID NO: 2407 - DNA, 24 bp, Homo sapiens
tatttttttgagacagtcttgctc

SEQ ID NO: 2408 - DNA, 23 bp, Homo sapiens
tgatgtgggaagcagagcagagc

SEQ ID NO: 2409 - DNA, 23 bp, Homo sapiens
caaccaggaagagcgagcccaga

SEQ ID NO: 2410 - DNA, 26 bp, Homo sapiens
cacaccctgacacaatgacgaatgaa

SEQ ID NO: 2411 - DNA, 26 bp, Homo sapiens
tggatggacccttcctcttcaccaag

SEQ ID NO: 2412 - DNA, 23 bp, Homo sapiens
ggtgggcatcttgtctctcagcc

SEQ ID NO: 2413 - DNA, 23 bp, Homo sapiens
gcagcgggagtttgtgtcttctg

SEQ ID NO: 2414 - DNA, 23 bp, Homo sapiens
gggacacagccaagccaaatcac

SEQ ID NO: 2415 - DNA, 24 bp, Homo sapiens
gccaaggcacagaaggcacagaag

SEQ ID NO: 2416 - DNA, 26 bp, Homo sapiens
ctattctctactccttttccacacac

SEQ ID NO: 2417 - DNA, 26 bp, Homo sapiens
cagagcctgtgaccctcagacttttg

SEQ ID NO: 2418 - DNA, 22 bp, Homo sapiens
caacctccgcctccagggttca

SEQ ID NO: 2419 - DNA, 22 bp, Homo sapiens
ctcactgcaacctccgcctccc

SEQ ID NO: 2420 - DNA, 22 bp, Homo sapiens
tggttcacatcagcaatctcaa

SEQ ID NO: 2421 - DNA, 22 bp, Homo sapiens
gaggtccaggctgcagtgggct

SEQ ID NO: 2422 - DNA, 23 bp, Homo sapiens
cgctctttccacactcacatccc

SEQ ID NO: 2423 - DNA, 23 bp, Homo sapiens
gtttgcccacagtctggaggagg

SEQ ID NO: 2424 - DNA, 26 bp, Homo sapiens
ccaaagtgagaaaatgtgacctacga

SEQ ID NO: 2425 - DNA, 25 bp, Homo sapiens
atgaagtgggcaggcagtgtgagtc

SEQ ID NO: 2426 - DNA, 24 bp, Homo sapiens
tgttgataataaacaatggctctt

SEQ ID NO: 2427 - DNA, 24 bp, Homo sapiens
gtgcaatggtgtgatctcggctca

SEQ ID NO: 2428 - DNA, 22 bp, Homo sapiens
gctaaggaacaagaattgcttg

SEQ ID NO: 2429 - DNA, 22 bp, Homo sapiens
ccaaggctcccactgggttgca

SEQ ID NO: 2430 - DNA, 23 bp, Homo sapiens
atgaactggaaggtctgggcgtc

SEQ ID NO: 2431 - DNA, 23 bp, Homo sapiens
ggacacaggaacccaggaccttc

SEQ ID NO: 2432 - DNA, 26 bp, Homo sapiens
cagtggagaaatggtatggtggaaac

SEQ ID NO: 2433 - DNA, 26 bp, Homo sapiens
cccacctcctttactacccaatgagc

SEQ ID NO: 2434 - DNA, 23 bp, Homo sapiens
caagaatatcacagacactccac

SEQ ID NO: 2435 - DNA, 23 bp, Homo sapiens
agcctcactttcttcaacggggg

SEQ ID NO: 2436 - DNA, 26 bp, Homo sapiens
acagtagtatccggtgctgtgggggc

SEQ ID NO: 2437 - DNA, 26 bp, Homo sapiens
gtttggatggatggatggatgaatgg

SEQ ID NO: 2438 - DNA, 22 bp, Homo sapiens
aatcaaacccgtgtaacgagtc

SEQ ID NO: 2439 - DNA, 22 bp, Homo sapiens
aaagatagacgaatgaatgatt

SEQ ID NO: 2440 - DNA, 23 bp, Homo sapiens
ggtgaacaatgtttacttcttaa

SEQ ID NO: 2441 - DNA, 23 bp, Homo sapiens
ctgtcgcccaggctggagtggag

SEQ ID NO: 2442 - DNA, 23 bp, Homo sapiens
ggctacacagagagattccctcg

SEQ ID NO: 2443 - DNA, 23 bp, Homo sapiens
gcagaagaggtgtcctgggctgt

SEQ ID NO: 2444 - DNA, 23 bp, Homo sapiens
ttgagaaatgaggtggaagtggg

SEQ ID NO: 2445 - DNA, 26 bp, Homo sapiens
tagggctggctgggaagcagaggcta

SEQ ID NO: 2446 - DNA, 26 bp, Homo sapiens
gtgacctgggagaaaagagggtgaga

SEQ ID NO: 2447 - DNA, 26 bp, Homo sapiens
gagagtgtaagaaggaaaataggtgg

SEQ ID NO: 2448 - DNA, 23 bp, Homo sapiens
cttgggaggttgaggcagaagaa

SEQ ID NO: 2449 - DNA, 26 bp, Homo sapiens
aaaatgctgggattacaggtgtgagc

SEQ ID NO: 2450 - DNA, 23 bp, Homo sapiens
gggacggaaaggaacaggaagca

SEQ ID NO: 2451 - DNA, 23 bp, Homo sapiens
gtgggagacaggtgtgagttggg

SEQ ID NO: 2452 - DNA, 22 bp, Homo sapiens
gtcttgtagtagtacaagaaca

SEQ ID NO: 2453 - DNA, 22 bp, Homo sapiens
aaacaaaggggttctgcaccat

SEQ ID NO: 2454 - DNA, 21 bp, Homo sapiens
tctgtcactgagacattgaga

SEQ ID NO: 2455 - DNA, 21 bp, Homo sapiens
acgtgcatggaaaagggctcc

SEQ ID NO: 2456 - DNA, 21 bp, Homo sapiens
ttggcctgttactgggtatag

SEQ ID NO: 2457 - DNA, 21 bp, Homo sapiens
actgattagctggcagcagtt

SEQ ID NO: 2458 - DNA, 26 bp, Homo sapiens
ataggtgagaacattttggctctgcc

SEQ ID NO: 2459 - DNA, 26 bp, Homo sapiens
gggtttgagggttttctggaatcccc

SEQ ID NO: 2460 - DNA, 26 bp, Homo sapiens
aagtctcacaggaagtcatgccacag

SEQ ID NO: 2461 - DNA, 26 bp, Homo sapiens
tgtggttcccaagtcatgtcagtgta

SEQ ID NO: 2462 - DNA, 23 bp, Homo sapiens
cgagggctacagtttccagtgag

SEQ ID NO: 2463 - DNA, 23 bp, Homo sapiens
aacacgccgccttgccagatgct

SEQ ID NO: 2464 - DNA, 26 bp, Homo sapiens
caaataacttgtccattaaaaacctt

SEQ ID NO: 2465 - DNA, 26 bp, Homo sapiens
ttccaatataaactaactacatggtc

SEQ ID NO: 2466 - DNA, 23 bp, Homo sapiens
gtgatttttagttagatgagtaa

SEQ ID NO: 2467 - DNA, 23 bp, Homo sapiens
catttcagtaaacctacattttg

SEQ ID NO: 2468 - DNA, 23 bp, Homo sapiens
aggctggagtgtggtggcaagat

SEQ ID NO: 2469 - DNA, 23 bp, Homo sapiens
tgaggcagggtggagcaggtgat

SEQ ID NO: 2470 - DNA, 23 bp, Homo sapiens
gggacactcacactccaggttgt

SEQ ID NO: 2471 - DNA, 23 bp, Homo sapiens
ccgcctgagtccctacatagtgc

SEQ ID NO: 2472 - DNA, 23 bp, Homo sapiens
gctggcacccctgctgagaagaa

SEQ ID NO: 2473 - DNA, 23 bp, Homo sapiens
gcagcagggaccgctatgaaatg

SEQ ID NO: 2474 - DNA, 21 bp, Homo sapiens
attagtggtgagatgacattc

SEQ ID NO: 2475 - DNA, 21 bp, Homo sapiens
atcgtatctacttccatgact

SEQ ID NO: 2476 - DNA, 21 bp, Homo sapiens
gaggctagggatggggggact

SEQ ID NO: 2477 - DNA, 21 bp, Homo sapiens
ataattctaaatattttgaaa

SEQ ID NO: 2478 - DNA, 23 bp, Homo sapiens
tgggagggtgacacatcctgagg

SEQ ID NO: 2479 - DNA, 23 bp, Homo sapiens
tacagcacaactgacctcccagc

SEQ ID NO: 2480 - DNA, 26 bp, Homo sapiens
gcacaagggcataatcagaaagccac

SEQ ID NO: 2481 - DNA, 26 bp, Homo sapiens
ctgaaacagggattgtatctccagag

SEQ ID NO: 2482 - DNA, 23 bp, Homo sapiens
gctgccttttgtcccatccccag

SEQ ID NO: 2483 - DNA, 23 bp, Homo sapiens
gaccaagggaggttcagagaagg

SEQ ID NO: 2484 - DNA, 22 bp, Homo sapiens
tgcaagactttattctaacaat

SEQ ID NO: 2485 - DNA, 22 bp, Homo sapiens
tcctggagaggcaggtgagagg

SEQ ID NO: 2486 - DNA, 26 bp, Homo sapiens
aatctttctcctctcagcgtgcttgc

SEQ ID NO: 2487 - DNA, 25 bp, Homo sapiens
gctgatgaaaactggattccctggc

SEQ ID NO: 2488 - DNA, 23 bp, Homo sapiens
tcacacacccgatgatggacagc

SEQ ID NO: 2489 - DNA, 23 bp, Homo sapiens
tcctacacctccgagcactccag

SEQ ID NO: 2490 - DNA, 26 bp, Homo sapiens
ggagaagggttgaactaagtaaggca

SEQ ID NO: 2491 - DNA, 26 bp, Homo sapiens
aatacagcacaactgacctcccagcc

SEQ ID NO: 2492 - DNA, 26 bp, Homo sapiens
ctcttccttttttttttttaggtgat

SEQ ID NO: 2493 - DNA, 26 bp, Homo sapiens
gaaaaaacactgtataccatcacaca

SEQ ID NO: 2494 - DNA, 23 bp, Homo sapiens
ctccattccctccccaatctccc

SEQ ID NO: 2495 - DNA, 23 bp, Homo sapiens
ggagccacagatggttctcactg

SEQ ID NO: 2496 - DNA, 26 bp, Homo sapiens
gggcctggcctagagtaggtgcttag

SEQ ID NO: 2497 - DNA, 26 bp, Homo sapiens
tgacaaatgaaccttcacaataagca

SEQ ID NO: 2498 - DNA, 25 bp, Homo sapiens
tttgtttttgaaaagtttttctggg

SEQ ID NO: 2499 - DNA, 25 bp, Homo sapiens
cctagttcagctaaagacggggttc

SEQ ID NO: 2500 - DNA, 26 bp, Homo sapiens
cattcagtccatcaggaacttgagtc

SEQ ID NO: 2501 - DNA, 26 bp, Homo sapiens
ctgtgtcgcccaggttagagtgcagt

SEQ ID NO: 2502 - DNA, 21 bp, Homo sapiens
tgaactcttgagctcaggcaa

SEQ ID NO: 2503 - DNA, 26 bp, Homo sapiens
caccacctggaggactacagcggagc

SEQ ID NO: 2504 - DNA, 21 bp, Homo sapiens
atacccctacaagcagctgcc

SEQ ID NO: 2505 - DNA, 28 bp, Homo sapiens
ggattgggcggagactgcttgatgtggg

SEQ ID NO: 2506 - DNA, 28 bp, Homo sapiens
gcacaccctgacacaatgacgaatgaat

SEQ ID NO: 2507 - DNA, 28 bp, Homo sapiens
ggtgggcatcttgtctctcagccatccc

SEQ ID NO: 2508 - DNA, 27 bp, Homo sapiens
tggcagaaggcaaaggaggagcaaagt

SEQ ID NO: 2509 - DNA, 28 bp, Homo sapiens
cctcgtttctattctctactccttttcc

SEQ ID NO: 2510 - DNA, 26 bp, Homo sapiens
ctggagtgcaatggtgccatctcagc

SEQ ID NO: 2511 - DNA, 21 bp, Homo sapiens
aaagaaaaataaataaatctt

SEQ ID NO: 2512 - DNA, 28 bp, Homo sapiens
ccccaccgcacctcctcacgaatcctga

SEQ ID NO: 2513 - DNA, 28 bp, Homo sapiens
cttccaaagtgagaaaatgtgacctacg

SEQ ID NO: 2514 - DNA, 24 bp, Homo sapiens
gtgtgtgatctatagacctatgta

SEQ ID NO: 2515 - DNA, 24 bp, Homo sapiens
cgcagtgagccgtgatggcaccac

SEQ ID NO: 2516 - DNA, 25 bp, Homo sapiens
gctcaccctggctgccctctattcc

SEQ ID NO: 2517 - DNA, 27 bp, Homo sapiens
gaatctttctcctctcagcgtgcttgc

SEQ ID NO: 2518 - DNA, 24 bp, Homo sapiens
acaatgagaaccagttgttcacaa

SEQ ID NO: 2519 - DNA, 24 bp, Homo sapiens
ctgggaacagtagtatccggtgct

SEQ ID NO: 2520 - DNA, 22 bp, Homo sapiens
gcgggaggaccaggagcagctc

SEQ ID NO: 2521 - DNA, 23 bp, Homo sapiens
atgtttacttcttaagagagcag

SEQ ID NO: 2522 - DNA, 27 bp, Homo sapiens
gctggcggcttccaaggctacacagag

SEQ ID NO: 2523 - DNA, 25 bp, Homo sapiens
ttttgagaaatgaggtggaagtggg

SEQ ID NO: 2524 - DNA, 25 bp, Homo sapiens
gctggctgcccactttgtgacctgg

SEQ ID NO: 2525 - DNA, 23 bp, Homo sapiens
agggaggctgaggcgggtgggat

SEQ ID NO: 2526 - DNA, 23 bp, Homo sapiens
gcacgcctctctgctgggtggca

SEQ ID NO: 2527 - DNA, 24 bp, Homo sapiens
atggtacttctgtgtggttttaat

SEQ ID NO: 2528 - DNA, 24 bp, Homo sapiens
ttgagacgttccaggctgtcttgc

SEQ ID NO: 2529 - DNA, 24 bp, Homo sapiens
gatgcagggtctgcctggtcaacg

SEQ ID NO: 2530 - DNA, 26 bp, Homo sapiens
ataggtgagaacattttggctctgcc

SEQ ID NO: 2531 - DNA, 23 bp, Homo sapiens
atgtgaaacatttggggcaatac

SEQ ID NO: 2532 - DNA, 28 bp, Homo sapiens
gggaccgagggctacagtttccagtgag

SEQ ID NO: 2533 - DNA, 25 bp, Homo sapiens
atcactgtaaaatgaaggcaatacc

SEQ ID NO: 2534 - DNA, 24 bp, Homo sapiens
tttctgggtactggtgatttttag

SEQ ID NO: 2535 - DNA, 24 bp, Homo sapiens
caggctggagtgtggtggcaagat

SEQ ID NO: 2536 - DNA, 28 bp, Homo sapiens
aagggcaggcaggaccagagaaccagcc

SEQ ID NO: 2537 - DNA, 23 bp, Homo sapiens
ggctgctggcacccctgctgaga

SEQ ID NO: 2538 - DNA, 25 bp, Homo sapiens
cttgtgaattctgatgcatgctctt

SEQ ID NO: 2539 - DNA, 21 bp, Homo sapiens
gcctggtgatcttccacacct

SEQ ID NO: 2540 - DNA, 28 bp, Homo sapiens
cgtgctgggagggtgacacatcctgagg

SEQ ID NO: 2541 - DNA, 28 bp, Homo sapiens
gcacaagggcataatcagaaagccacaa

SEQ ID NO: 2542 - DNA, 27 bp, Homo sapiens
ggaaaccaaggcacagagagggacagg

SEQ ID NO: 2543 - DNA, 24 bp, Homo sapiens
caaaatacattttcaagctggctg

SEQ ID NO: 2544 - DNA, 27 bp, Homo sapiens
gaatctttctcctctcagcgtgcttgc

SEQ ID NO: 2545 - DNA, 23 bp, Homo sapiens
tggacagcgggacctcgtggtgc

SEQ ID NO: 2546 - DNA, 28 bp, Homo sapiens
cctctacaactgctaatgggagaagggt

SEQ ID NO: 2547 - DNA, 22 bp, Homo sapiens
tttgcctgacaccgagtggccc

SEQ ID NO: 2548 - DNA, 28 bp, Homo sapiens
cgtctctccattccctccccaatctccc

SEQ ID NO: 2549 - DNA, 22 bp, Homo sapiens
agctggcactgccagtgcacag

SEQ ID NO: 2550 - DNA, 21 bp, Homo sapiens
tgtcatttgtttttgaaaagt

SEQ ID NO: 2551 - DNA, 22 bp, Homo sapiens
cctatggaacgcgtctagaatc

SEQ ID NO: 2552 - DNA, 21 bp, Homo sapiens
tagactgggagataaaatatg

SEQ ID NO: 2553 - DNA, 40 bp, Homo sapiens
ttactgatcttttgatcatcgacctcctagccctgtctca

SEQ ID NO: 2554 - DNA, 28 bp, Homo sapiens
ccacagcacctttctatggttccaacag

SEQ ID NO: 2555 - DNA, 32 bp, Homo sapiens
agcacaggccccgttcctctctgttcgattac

SEQ ID NO: 2556 - DNA, 21 bp, Homo sapiens
ttgtccaagctgatgtgtagt

SEQ ID NO: 2557 - DNA, 40 bp, Homo sapiens
tcctgggagtcttctcggtcgagaccagcatggccaacaa

SEQ ID NO: 2558 - DNA, 28 bp, Homo sapiens
gctgagtgaggaccagatgagccaggag

SEQ ID NO: 2559 - DNA, 32 bp, Homo sapiens
acacttgacaccaccaccacctcgagaaggtc

SEQ ID NO: 2560 - DNA, 28 bp, Homo sapiens
ctgactggatggacccttcctcttcacc

SEQ ID NO: 2561 - DNA, 36 bp, Homo sapiens
tgacacaaagatattctgctttgccagctcgaagga

SEQ ID NO: 2562 - DNA, 27 bp, Homo sapiens
agaagttgatgcgaggaagggcggcgg

SEQ ID NO: 2563 - DNA, 38 bp, Homo sapiens
tggtcatacctgttgttcgaattctgaataccgtcccc

SEQ ID NO: 2564 - DNA, 28 bp, Homo sapiens
gccaaggcacagaaggcacagaagtgac

SEQ ID NO: 2565 - DNA, 30 bp, Homo sapiens
agagggagactctgtctcgatgccctgcct

SEQ ID NO: 2566 - DNA, 28 bp, Homo sapiens
tcagagcctgtgaccctcagacttttga

SEQ ID NO: 2567 - DNA, 38 bp, Homo sapiens
tgtatttctaaatcgaagcagggaagggagaaaaggcc

SEQ ID NO: 2568 - DNA, 26 bp, Homo sapiens
actttgtgatagtttttttcccccca

SEQ ID NO: 2569 - DNA, 40 bp, Homo sapiens
atgttggtcaggctggtctcgagatcagcctggccaacgt

SEQ ID NO: 2570 - DNA, 21 bp, Homo sapiens
tgcagtgggctgataccgtct

SEQ ID NO: 2571 - DNA, 40 bp, Homo sapiens
atctctaaaagaaatgtttcgactctccacgcatctctgc

SEQ ID NO: 2572 - DNA, 27 bp, Homo sapiens
gggaatctacaaacccaccctctgccg

SEQ ID NO: 2573 - DNA, 33 bp, Homo sapiens
tctggactcctcgatctgcatcttgactctgcc

SEQ ID NO: 2574 - DNA, 27 bp, Homo sapiens
atgaagtgggcaggcagtgtgagtcca

SEQ ID NO: 2575 - DNA, 32 bp, Homo sapiens
tcccgctcttaaacctcgagccccttactctc

SEQ ID NO: 2576 - DNA, 24 bp, Homo sapiens
tttttgagacagggttttgctctt

SEQ ID NO: 2577 - DNA, 40 bp, Homo sapiens
ctcctgcttgttgaggtttcgagacaagcctggccaacat

SEQ ID NO: 2578 - DNA, 24 bp, Homo sapiens
agctgggctgccagcagacatggg

SEQ ID NO: 2579 - DNA, 40 bp, Homo sapiens
cttacacagaatacctactcgacctagggagacaagtcct

SEQ ID NO: 2580 - DNA, 28 bp, Homo sapiens
gcccaacaggacacaggaacccaggacc

SEQ ID NO: 2581 - DNA, 35 bp, Homo sapiens
aggaagcgttgttacccaggagatcgagtggtttg

SEQ ID NO: 2582 - DNA, 27 bp, Homo sapiens
ccgtcacttcctgtctctcccacctcc

SEQ ID NO: 2583 - DNA, 40 bp, Homo sapiens
aatgaaaaggttggtcgaagatactttcagattatagcct

SEQ ID NO: 2584 - DNA, 24 bp, Homo sapiens
tgtgaccgttgtcttgattcctgg

SEQ ID NO: 2585 - DNA, 40 bp, Homo sapiens
aagactcccagaggcacttcgaagacccacttcctacgtc

SEQ ID NO: 2586 - DNA, 24 bp, Homo sapiens
gtttggatggatggatggatgaat

SEQ ID NO: 2587 - DNA, 40 bp, Homo sapiens
ttattagcgaagtcacgttcgatccatccatccattcatc

SEQ ID NO: 2588 - DNA, 22 bp, Homo sapiens
gaatgaatgattgatggatgat

SEQ ID NO: 2589 - DNA, 40 bp, Homo sapiens
gcggccgcgggctcagaatcgatccatccatccatcatcc

SEQ ID NO: 2590 - DNA, 23 bp, Homo sapiens
tttttttttttaagtaagacgga

SEQ ID NO: 2591 - DNA, 40 bp, Homo sapiens
tgcaccaccggggtgtcctcgaaaccaacctgaccaacat

SEQ ID NO: 2592 - DNA, 28 bp, Homo sapiens
gtccagtgggtctcacctcagagtcagc

SEQ ID NO: 2593 - DNA, 35 bp, Homo sapiens
tcaaggactgcgccctctcagattctttcgactta

SEQ ID NO: 2594 - DNA, 24 bp, Homo sapiens
gggaaggaggtagggctggctggg

SEQ ID NO: 2595 - DNA, 30 bp, Homo sapiens
agcctgaccaggaaggctgccttcgacaca

SEQ ID NO: 2596 - DNA, 28 bp, Homo sapiens
ccctcctgccaccctcctactccttacc

SEQ ID NO: 2597 - DNA, 34 bp, Homo sapiens
acttgttgacttctaagtcgagggagccagaggg

SEQ ID NO: 2598 - DNA, 27 bp, Homo sapiens
caaaatgctgggattacaggtgtgagc

SEQ ID NO: 2599 - DNA, 32 bp, Homo sapiens
tgcttgggacatgagggcttcccctcgatatc

SEQ ID NO: 2600 - DNA, 28 bp, Homo sapiens
tgagatgggctccgtgtcccctgctgga

SEQ ID NO: 2601 - DNA, 32 bp, Homo sapiens
tgcaccttaaatcgatccacctgacccctgcc

SEQ ID NO: 2602 - DNA, 24 bp, Homo sapiens
caccttaagtaaaggaaaaagcgt

SEQ ID NO: 2603 - DNA, 40 bp, Homo sapiens
tctattttattccatttatcgacaggaaggaaggaggaag

SEQ ID NO: 2604 - DNA, 24 bp, Homo sapiens
accctctgggttctttctccacgt

SEQ ID NO: 2605 - DNA, 40 bp, Homo sapiens
cactcccttccacttcaatcgattcacacaccattcctcc

SEQ ID NO: 2606 - DNA, 24 bp, Homo sapiens
ggcagcagttaaaatcatcattag

SEQ ID NO: 2607 - DNA, 40 bp, Homo sapiens
atgtcctttaatggacattcgaacactttacactcagttc

SEQ ID NO: 2608 - DNA, 28 bp, Homo sapiens
gggtttgagggttttctggaatccccac

SEQ ID NO: 2609 - DNA, 37 bp, Homo sapiens
agagaagtaaatcgatcttcagcaccctgttcccagg

SEQ ID NO: 2610 - DNA, 23 bp, Homo sapiens
caataatttgtgaaaatggactt

SEQ ID NO: 2611 - DNA, 40 bp, Homo sapiens
ttacttatgagaatattctcgatctgaccttgtgatccac

SEQ ID NO: 2612 - DNA, 27 bp, Homo sapiens
acggaacacgccgccttgccagatgct

SEQ ID NO: 2613 - DNA, 32 bp, Homo sapiens
tgaagacttcgaggaggaggttgagcggactc

SEQ ID NO: 2614 - DNA, 25 bp, Homo sapiens
aacaggttccaatataaactaacta

SEQ ID NO: 2615 - DNA, 40 bp, Homo sapiens
ctgacagtttttatattttcgaatacctgacctcgtgatc

SEQ ID NO: 2616 - DNA, 24 bp, Homo sapiens
atttcagtaaacctacattttgtt

SEQ ID NO: 2617 - DNA, 40 bp, Homo sapiens
cccttccaaccttccccttcgattcatgtcagtgacttgc

SEQ ID NO: 2618 - DNA, 25 bp, Homo sapiens
ggaatgaggcagggtggagcaggtg

SEQ ID NO: 2619 - DNA, 32 bp, Homo sapiens
aggctgatttcgatcacctgctccaccctgac

SEQ ID NO: 2620 - DNA, 23 bp, Homo sapiens
ccacccgcaccgcacaccagttg

SEQ ID NO: 2621 - DNA, 31 bp, Homo sapiens
accatttccccactgctcgactccccagtct

SEQ ID NO: 2622 - DNA, 28 bp, Homo sapiens
gttcaaggagcagtgagggcacccaggg

SEQ ID NO: 2623 - DNA, 32 bp, Homo sapiens
tgccaaactacaggcttattcgaccgcccacc

SEQ ID NO: 2624 - DNA, 25 bp, Homo sapiens
atcgtatctacttccatgactgcat

SEQ ID NO: 2625 - DNA, 40 bp, Homo sapiens
actgtaattgctagatgttcgaactcctgacctcctgatc

SEQ ID NO: 2626 - DNA, 21 bp, Homo sapiens
tttctgttattttctttttga

SEQ ID NO: 2627 - DNA, 40 bp, Homo sapiens
tcccttcaatgatgaggctcgaccaatttcaaatgtatat

SEQ ID NO: 2628 - DNA, 23 bp, Homo sapiens
cccaggctcccttgtccgtctcc

SEQ ID NO: 2629 - DNA, 32 bp, Homo sapiens
tgcaacaggtgcctgaatcgaagatcctcccc

SEQ ID NO: 2630 - DNA, 28 bp, Homo sapiens
gaggaactgaaagatttgggaacaacct

SEQ ID NO: 2631 - DNA, 37 bp, Homo sapiens
tccatcttctcccttgccctctattattttcgatctg

SEQ ID NO: 2632 - DNA, 27 bp, Homo sapiens
gaggtccacagagaggagacacaaggc

SEQ ID NO: 2633 - DNA, 38 bp, Homo sapiens
aggactctgcctacaccttgactcgataaatggtagtg

SEQ ID NO: 2634 - DNA, 24 bp, Homo sapiens
cctggagaggcaggtgagagggca

SEQ ID NO: 2635 - DNA, 40 bp, Homo sapiens
atttttttctttcacacttcgaccccaccccaccacctcc

SEQ ID NO: 2636 - DNA, 28 bp, Homo sapiens
gctgagaggaagaagccagtagagaaga

SEQ ID NO: 2637 - DNA, 39 bp, Homo sapiens
aatgaaaaggttggtcgattcagagaaaatcacagattg

SEQ ID NO: 2638 - DNA, 28 bp, Homo sapiens
gacaggacaaggacccacaggcaccagc

SEQ ID NO: 2639 - DNA, 35 bp, Homo sapiens
acaagggtgagttgtgcgtatttcgacctccttcc

SEQ ID NO: 2640 - DNA, 24 bp, Homo sapiens
gcccaggctcccttgtccgtctcc

SEQ ID NO: 2641 - DNA, 32 bp, Homo sapiens
aataattctcgaagatcctccccacccgcccc

SEQ ID NO: 2642 - DNA, 22 bp, Homo sapiens
tatataaagcaaacaagtttca

SEQ ID NO: 2643 - DNA, 40 bp, Homo sapiens
cacctgactgggagcagctcgagtccagtatttttccaat

SEQ ID NO: 2644 - DNA, 28 bp, Homo sapiens
gcacaggacggagccacagatggttctc

SEQ ID NO: 2645 - DNA, 33 bp, Homo sapiens
tggaagggatggtgtgttctcgaggttgcagag

SEQ ID NO: 2646 - DNA, 22 bp, Homo sapiens
agcaaaaccaaaccatcttttt

SEQ ID NO: 2647 - DNA, 40 bp, Homo sapiens
aggaggtcttctgaatcctcgatgactgtgagaaactttt

SEQ ID NO: 2648 - DNA, 21 bp, Homo sapiens
acggggttccttgtcccactg

SEQ ID NO: 2649 - DNA, 40 bp, Homo sapiens
cccttccaaccttccccttcgaactgctgagtaggaagcg

SEQ ID NO: 2650 - DNA, 22 bp, Homo sapiens
tttttttgagacggagtctcgc

SEQ ID NO: 2651 - DNA, 40 bp, Homo sapiens
acacggcaaccagacacgtcgagaccatcctggctaacac

SEQ ID NO: 2652 - DNA, 40 bp, Homo sapiens
tgagacagggctaggaggtcgatgatcaaaagatcagtaa

SEQ ID NO: 2653 - DNA, 32 bp, Homo sapiens
gtaatcgaacagagaggaacggggcctgtgct

SEQ ID NO: 2654 - DNA, 40 bp, Homo sapiens
ttgttggccatgctggtctcgaccgagaagactcccagga

SEQ ID NO: 2655 - DNA, 32 bp, Homo sapiens
tggaccttctcgaggtggtggtggtgtcaagt

SEQ ID NO: 2656 - DNA, 37 bp, Homo sapiens
tccttcgagctggcaaagcagaatatctttgtgtcag

SEQ ID NO: 2657 - DNA, 38 bp, Homo sapiens
agaattcgaacaacaggtatgaccaatgagagtagcgg

SEQ ID NO: 2658 - DNA, 30 bp, Homo sapiens
aggcagggcatcgagacagagtctccctct

SEQ ID NO: 2659 - DNA, 33 bp, Homo sapiens
aggccttttctcccttccctgcttcgatttaga

SEQ ID NO: 2660 - DNA, 40 bp, Homo sapiens
acgttggccaggctgatctcgagaccagcctgaccaacat

SEQ ID NO: 2661 - DNA, 40 bp, Homo sapiens
gcagagatgcgtggagagtcgaaacatttcttttagagat

SEQ ID NO: 2662 - DNA, 33 bp, Homo sapiens
agagtcaagatgcagatcgaggagtccagaggg

SEQ ID NO: 2663 - DNA, 32 bp, Homo sapiens
gagagtaaggggctcgaggtttaagagcggga

SEQ ID NO: 2664 - DNA, 40 bp, Homo sapiens
atgttggccaggcttgtctcgaaacctcaacaagcaggag

SEQ ID NO: 2665 - DNA, 40 bp, Homo sapiens
aggacttgtctccctaggtcgagtaggtattctgtgtaag

SEQ ID NO: 2666 - DNA, 34 bp, Homo sapiens
accactcgatctcctgggtaacaacgcttccttc

SEQ ID NO: 2667 - DNA, 39 bp, Homo sapiens
tctgaaagtatcttcgaccaaccttttcattccccagtg

SEQ ID NO: 2668 - DNA, 40 bp, Homo sapiens
gacgtaggaagtgggtcttcgaagtgcctctgggagtctt

SEQ ID NO: 2669 - DNA, 40 bp, Homo sapiens
gatgaatggatggatggatcgaacgtgacttcgctaataa

SEQ ID NO: 2670 - DNA, 40 bp, Homo sapiens
ggatgatggatggatggatcgattctgagcccgcggccgc

SEQ ID NO: 2671 - DNA, 40 bp, Homo sapiens
atgttggtcaggttggtttcgaggacaccccggtggtgca

SEQ ID NO: 2672 - DNA, 36 bp, Homo sapiens
taagtcgaaagaatctgagagggcgcagtccttgac

SEQ ID NO: 2673 - DNA, 30 bp, Homo sapiens
tgtgtcgaaggcagccttcctggtcaggct

SEQ ID NO: 2674 - DNA, 37 bp, Homo sapiens
tccctcgacttagaagtcaacaagtatgaagtcaggc

SEQ ID NO: 2675 - DNA, 32 bp, Homo sapiens
gatatcgaggggaagccctcatgtcccaagca

SEQ ID NO: 2676 - DNA, 25 bp, Homo sapiens
tcaggtggatcgatttaaggtgcac

SEQ ID NO: 2677 - DNA, 40 bp, Homo sapiens
cttcctccttccttcctgtcgataaatggaataaaataga

SEQ ID NO: 2678 - DNA, 40 bp, Homo sapiens
ggaggaatggtgtgtgaatcgattgaagtggaagggagtg

SEQ ID NO: 2679 - DNA, 40 bp, Homo sapiens
gaactgagtgtaaagtgttcgaatgtccattaaaggacat

SEQ ID NO: 2680 - DNA, 37 bp, Homo sapiens
tcctgggaacagggtgctgaagatcgatttacttctc

SEQ ID NO: 2681 - DNA, 40 bp, Homo sapiens
gtggatcacaaggtcagatcgagaatattctcataagtaa

SEQ ID NO: 2682 - DNA, 33 bp, Homo sapiens
tccgctcaacctcctcctcgaagtcttcatagc

SEQ ID NO: 2683 - DNA, 40 bp, Homo sapiens
gatcacgaggtcaggtattcgaaaatataaaaactgtcag

SEQ ID NO: 2684 - DNA, 40 bp, Homo sapiens
gcaagtcactgacatgaatcgaaggggaaggttggaaggg

SEQ ID NO: 2685 - DNA, 32 bp, Homo sapiens
agggtggagcaggtgatcgaaatcagcctagg

SEQ ID NO: 2686 - DNA, 31 bp, Homo sapiens
agactggggagtcgagcagtggggaaatggt

SEQ ID NO: 2687 - DNA, 32 bp, Homo sapiens
tgggcggtcgaataagcctgtagtttggcaaa

SEQ ID NO: 2688 - DNA, 40 bp, Homo sapiens
gatcaggaggtcaggagttcgaacatctagcaattacagt

SEQ ID NO: 2689 - DNA, 40 bp, Homo sapiens
atatacatttgaaattggtcgagcctcatcattgaaggga

SEQ ID NO: 2690 - DNA, 32 bp, Homo sapiens
atcttcgattcaggcacctgttgcaagccagg

SEQ ID NO: 2691 - DNA, 37 bp, Homo sapiens
acagatcgaaaataatagagggcaagggagaagatgg

SEQ ID NO: 2692 - DNA, 38 bp, Homo sapiens
acactaccatttatcgagtcaaggtgtaggcagagtcc

SEQ ID NO: 2693 - DNA, 40 bp, Homo sapiens
ggaggtggtggggtggggtcgaagtgtgaaagaaaaaaat

SEQ ID NO: 2694 - DNA, 37 bp, Homo sapiens
tctctgaatcgaccaaccttttcattccccagtgttg

SEQ ID NO: 2695 - DNA, 35 bp, Homo sapiens
aggaaggaggtcgaaatacgcacaactcacccttg

SEQ ID NO: 2696 - DNA, 38 bp, Homo sapiens
aggatcttcgagaattattgggtagtttaatagtcacc

SEQ ID NO: 2697 - DNA, 40 bp, Homo sapiens
attggaaaaatactggactcgagctgctcccagtcaggtg

SEQ ID NO: 2698 - DNA, 33 bp, Homo sapiens
tctgcaacctcgagaacacaccatcccttccag

SEQ ID NO: 2699 - DNA, 40 bp, Homo sapiens
aaaagtttctcacagtcatcgaggattcagaagacctcct

SEQ ID NO: 2700 - DNA, 40 bp, Homo sapiens
cgcttcctactcagcagttcgaaggggaaggttggaaggg

SEQ ID NO: 2701 - DNA, 40 bp, Homo sapiens
gtgttagccaggatggtctcgacgtgtctggttgccgtgt

SEQ ID NO: 2702 - DNA, 60 bp, Homo sapiens; identical to SEQ ID NO: 463

SEQ ID NO: 2703 - DNA, 60 bp, Homo sapiens; identical to SEQ ID NO: 467

SEQ ID NO: 2704 - DNA, 60 bp, Homo sapiens; identical to SEQ ID NO: 481

SEQ ID NO: 2705 - DNA, 60 bp, Homo sapiens; identical to SEQ ID NO: 478

SEQ ID NO: 2706 - DNA, 60 bp, Homo sapiens; identical to SEQ ID NO: 489

SEQ ID NO: 2707 - DNA, 60 bp, Homo sapiens
caggagggtagcccattgggtacctagctcgagttccaggcttagctctggagctgtgtg

SEQ ID NO: 2708 - DNA, 60 bp, Homo sapiens
agctcaggcaatccatgcttggagaaagtcgatttccccattttggccgatggcatcgct

SEQ ID NO: 2709 - DNA, 60 bp, Homo sapiens; identical to SEQ ID NO: 486

SEQ ID NO: 2710 - DNA, 60 bp, Homo sapiens
attttatctccaggtaaaaaggggtgtgtcgaagtgctctgtggtgatgcagcaggttga

SEQ ID NO: 2711 - DNA, 60 bp, Homo sapiens; identical to SEQ ID NO: 485

SEQ ID NO: 2712 - DNA, 60 bp, Homo sapiens; identical to SEQ ID NO: 497

SEQ ID NO: 2713 - DNA, 60 bp, Homo sapiens
gtccgaatcgttgggtttggcggagggctcgataacttagaaaatgtgaatgctagagac

SEQ ID NO: 2714 - DNA, 60 bp, Homo sapiens
aaggcaggcagatcacgaggccaggagttcgatagataatagatagaaatatgcagaaag

SEQ ID NO: 2715 - DNA, 60 bp, Homo sapiens
agttttttcatctgaaagacaaacgaggtcgagctgccgtgtgtcccatagccaccagct

SEQ ID NO: 2716 - DNA, 60 bp, Homo sapiens
tctagctcagggtttgtgaatgcaccaatcgaggtgaactgtggcaatgaaaaccagaaa

SEQ ID NO: 2717 - DNA, 60 bp, Homo sapiens
ctgccgcctaaggttgttgatgtagctctcgaacgtcttgtgtaagaatggttttaatga

SEQ ID NO: 2718 - DNA, 60 bp, Homo sapiens
ggtgagtggcagatagctgggaaaacattcgataaccccaaattttccagcccctcaccc

SEQ ID NO: 2719 - DNA, 60 bp, Homo sapiens
gttcaaatcaagctaggaggcatccgtgtcgaaagaaaaagatacagagagttcctatat

SEQ ID NO: 2720 - DNA, 60 bp, Homo sapiens
gatggcatcagggtacgagggggagacctcgacaccaacatatatctgtgattcactctg

SEQ ID NO: 2721 - DNA, 60 bp, Homo sapiens
caggcaggcagaatgcaccatcaatgaatcgattctgatgacatggttgttcctcctgtt

SEQ ID NO: 2722 - DNA, 60 bp, Homo sapiens
gccggcccatagctcagtcatgtgaacctcgaaaaagaaagaaacatttcaaaatattga

SEQ ID NO: 2723 - DNA, 60 bp, Homo sapiens
tttagctcaaggtttgtgaatgcaccaatcgaacagtccaaataaagtctggaaagaaac

SEQ ID NO: 2724 - DNA, 60 bp, Homo sapiens
cgcagcagttcgcttagggatgcagacgtcgaatagtccactgttactggcttactgctt

SEQ ID NO: 2725 - DNA, 60 bp, Homo sapiens
ggcaggcagatcacttgagctcaggagttcgatatctgtttacttaaccacaaagataac

SEQ ID NO: 2726 - DNA, 60 bp, Homo sapiens
tttagctcaaggtttgtgaatgcaccaatcgatattttacttaaccatagaacaattact

SEQ ID NO: 2727 - DNA, 60 bp, Homo sapiens
tgatctgcccaacaagagagagctgggatcgaatgcaacattcatttactacatgaccat

SEQ ID NO: 2728 - DNA, 60 bp, Homo sapiens
ctatacatcctccaaggtgatgataacttcgagagtgtttttaggaggctggtgctgaca

SEQ ID NO: 2729 - DNA, 60 bp, Homo sapiens
attcataggcacgggtaccaagactacttcgatgctgcattttagaacattccagcaaaa

SEQ ID NO: 2730 - DNA, 60 bp, Homo sapiens
ggcaggcagatcacttgaggtcaagagttcgagtcaaagtgatgctccatgaaaaaagag

SEQ ID NO: 2731 - DNA, 60 bp, Homo sapiens
taacaggtttaaaacaaaaatttaaaactcgagggggcgtagttctgccggggtttcagc

SEQ ID NO: 2732 - DNA, 60 bp, Homo sapiens
tggaaacgggatatcttcatataaaatctcgaaatatcctcttgcagatattacaaaaag

SEQ ID NO: 2733 - DNA, 60 bp, Homo sapiens
agaggtgtcctccgtgggtcaggaagggtcgacaatcatgttgagcaaatgaattaaaaa

SEQ ID NO: 2734 - DNA, 60 bp, Homo sapiens
gccggcccatagctcagtcatgtgaacctcgacagtaggtatttttggaagctcagattt

SEQ ID NO: 2735 - DNA, 60 bp, Homo sapiens
ggcaggaggatcacttgaggccaagaattcgattcgtccaattatgagaacatgctgcac

SEQ ID NO: 2736 - DNA, 60 bp, Homo sapiens
ttacaaaccttgagctagatacagagtgtcgaaatcagcttgaggtcttggggactgagt

SEQ ID NO: 2737 - DNA, 60 bp, Homo sapiens
gttcaaatcaagctaggaggcatccgtgtcgaattttcatggcagccctcttttgatatc

SEQ ID NO: 2738 - DNA, 60 bp, Homo sapiens
cgccaggtttagcgtgaggcgctccaggtcgagtggttgacctggaagcaaaattaaaag

SEQ ID NO: 2739 - DNA, 60 bp, Homo sapiens
tggaaacgggatatcttcatataaaatctcgaaatatcctcttgcagattttacaaaaag

SEQ ID NO: 2740 - DNA, 60 bp, Homo sapiens
gtctcagcctaagagcaagagcttaagttcgagcagtgtggaaccttcgggagctgtgtg

SEQ ID NO: 2741 - DNA, 60 bp, Homo sapiens
gtgggcagatgacttgagcttcaggagttcgagaccagtttggccaacaaagcaaaaccg

SEQ ID NO: 2742 - DNA, 60 bp, Homo sapiens
gtttgacttggagccttctcattgcaggtcgaagtgctctgtggtgatgcagcaggttga

SEQ ID NO: 2743 - DNA, 60 bp, Homo sapiens
gtggtggtacaggtgctatgcttatgactcgagatctccactcagataccaaaatctgta

SEQ ID NO: 2744 - DNA, 60 bp, Homo sapiens
gatggcatcagggtacgagggggagacctcgatgagcagcatgttatttactggctcctg

SEQ ID NO: 2745 - DNA, 60 bp, Homo sapiens
aatgaagtcccaggctttcgtctcacgttcgacagcctaaatctctgaagccgtctcctg

SEQ ID NO: 2746 - DNA, 60 bp, Homo sapiens
taaagctatgcataagactaaggttctgtcgaaccacctttatttactgtatatcagcta

SEQ ID NO: 2747 - DNA, 60 bp, Homo sapiens
cactgcactcatcaataacatcttcaactcgaagtgctctgtggtgatgcagcaggttga

SEQ ID NO: 2748 - DNA, 60 bp, Homo sapiens
ggcaggtggatcacttgagctcaggagttcgaaacccaacataaatgcaaataaataaat

SEQ ID NO: 2749 - DNA, 60 bp, Homo sapiens
ggcaggtggatcacttgaggccacgagttcgagtactcagatatttacttacctactcaa

SEQ ID NO: 2750 - DNA, 60 bp, Homo sapiens
ggcgggaggatcacctaagctcaggagttcgagactagcttggccaacatggtaaaatcc

SEQ ID NO: 2751 - DNA, 60 bp, Homo sapiens
caggagggtagcccattgggtacctagctcgaatgcaggctgttgttggaagatccgtga

SEQ ID NO: 2752 - DNA, 23 bp, Homo sapiens
caagtgtgggggcgagtgttgcg

SEQ ID NO: 2753 - DNA, 23 bp, Homo sapiens
tgcacacccaggacctggaaaaa

SEQ ID NO: 2754 - DNA, 26 bp, Homo sapiens
ccgttgctcaagaaccatccagagtc

SEQ ID NO: 2755 - DNA, 26 bp, Homo sapiens
gcaaacttcaggctgtattgttctcg

SEQ ID NO: 2756 - DNA, 26 bp, Homo sapiens
ttatcccctgtctgacttgttccccg

SEQ ID NO: 2757 - DNA, 26 bp, Homo sapiens
gcgttacatctgttgtgccagcaaaa

SEQ ID NO: 2758 - DNA, 26 bp, Homo sapiens
cagtagttctaaaagagaggatggta

SEQ ID NO: 2759 - DNA, 26 bp, Homo sapiens
gccagtaaaccaaggcttattaggat

SEQ ID NO: 2760 - DNA, 23 bp, Homo sapiens
gcgggagacagagaaaggaaaag

SEQ ID NO: 2761 - DNA, 23 bp, Homo sapiens
caggcagaggtggaccgagaacc

SEQ ID NO: 2762 - DNA, 26 bp, Homo sapiens
cattcccagcaggagggtaaaggctt

SEQ ID NO: 2763 - DNA, 26 bp, Homo sapiens
acaacaacaataacagacaccagggt

SEQ ID NO: 2764 - DNA, 23 bp, Homo sapiens
tatcccctgtctgactcgttccc

SEQ ID NO: 2765 - DNA, 23 bp, Homo sapiens
tttggaatgcggtggcgtctggc

SEQ ID NO: 2766 - DNA, 21 bp, Homo sapiens
tggaaaggactcccaaattca

SEQ ID NO: 2767 - DNA, 21 bp, Homo sapiens
taaaatcagtaatctaagctt

SEQ ID NO: 2768 - DNA, 23 bp, Homo sapiens
gctacatctgctcaaccgtcctg

SEQ ID NO: 2769 - DNA, 23 bp, Homo sapiens
cccacactgccaagcccttacca

SEQ ID NO: 2770 - DNA, 26 bp, Homo sapiens
aacatcagtcaggaaagacaaggttc

SEQ ID NO: 2771 - DNA, 26 bp, Homo sapiens
tggtgtgtcctgggttttgtggagca

SEQ ID NO: 2772 - DNA, 25 bp, Homo sapiens
aaagtgagagaagggtcctgtagca

SEQ ID NO: 2773 - DNA, 25 bp, Homo sapiens
ttcacatgttcagtgtcttcctttt

SEQ ID NO: 2774 - DNA, 26 bp, Homo sapiens
tggctgatgagcgtgtccgcatagtt

SEQ ID NO: 2775 - DNA, 26 bp, Homo sapiens
ctccgaacacaaagcatacataccct

SEQ ID NO: 2776 - DNA, 23 bp, Homo sapiens
aaagtaatatcttcccaatgacc

SEQ ID NO: 2777 - DNA, 23 bp, Homo sapiens
tctgacctcactttctcacctct

SEQ ID NO: 2778 - DNA, 26 bp, Homo sapiens
aagtatgatgtagaaaagccttgagc

SEQ ID NO: 2779 - DNA, 26 bp, Homo sapiens
atggatgggcagtggaaataaggcag

SEQ ID NO: 2780 - DNA, 21 bp, Homo sapiens
ctactctggtgggaccttgga

SEQ ID NO: 2781 - DNA, 21 bp, Homo sapiens
gtctagactacaacacaaagt

SEQ ID NO: 2782 - DNA, 23 bp, Homo sapiens
gggtgggagttgctcacttgttc

SEQ ID NO: 2783 - DNA, 23 bp, Homo sapiens
gcctggcacacggtgggaaacaa

SEQ ID NO: 2784 - DNA, 26 bp, Homo sapiens
cagtagttctaaaagagaggatggta

SEQ ID NO: 2785 - DNA, 26 bp, Homo sapiens
ccaggaaacaggaagagttctcaagc

SEQ ID NO: 2786 - DNA, 23 bp, Homo sapiens
tggaagtgctgaactggagcccc

SEQ ID NO: 2787 - DNA, 23 bp, Homo sapiens
gaggaataggcagagcacagcag

SEQ ID NO: 2788 - DNA, 26 bp, Homo sapiens
gccacttatcactggggacacctgaa

SEQ ID NO: 2789 - DNA, 26 bp, Homo sapiens
acatcattagtttctacattctacaa

SEQ ID NO: 2790 - DNA, 21 bp, Homo sapiens
actccctaaaatcaaacaaca

SEQ ID NO: 2791 - DNA, 21 bp, Homo sapiens
agatgcttccctgttgtatta

SEQ ID NO: 2792 - DNA, 22 bp, Homo sapiens
cctctttggccagagccaaaac

SEQ ID NO: 2793 - DNA, 22 bp, Homo sapiens
cttgatttggttttccttttct

SEQ ID NO: 2794 - DNA, 21 bp, Homo sapiens
attgtaaacacaccaatcggc

SEQ ID NO: 2795 - DNA, 21 bp, Homo sapiens
tccttccttagctgagatccc

SEQ ID NO: 2796 - DNA, 26 bp, Homo sapiens
atggcacctgccgcagctcaggtggg

SEQ ID NO: 2797 - DNA, 26 bp, Homo sapiens
tgtatctcaatggattctctcattat

SEQ ID NO: 2798 - DNA, 26 bp, Homo sapiens
gggtaacttgtagacactgctgcctc

SEQ ID NO: 2799 - DNA, 26 bp, Homo sapiens
gcccactgagaaatagagatgtcttg

SEQ ID NO: 2800 - DNA, 24 bp, Homo sapiens
tgtgtatctagctgctctggtggg

SEQ ID NO: 2801 - DNA, 24 bp, Homo sapiens
tggattggatcatgaagcagaagg

SEQ ID NO: 2802 - DNA, 23 bp, Homo sapiens
aggtggaggtcagtgatgctcgg

SEQ ID NO: 2803 - DNA, 23 bp, Homo sapiens
gaacatccctcacttgcccagtc

SEQ ID NO: 2804 - DNA, 23 bp, Homo sapiens
tggtggttcataggcaggctgag

SEQ ID NO: 2805 - DNA, 23 bp, Homo sapiens
atgatggctgtgcctgggtgctg

SEQ ID NO: 2806 - DNA, 23 bp, Homo sapiens
atggaggctgcccctgtaacaca

SEQ ID NO: 2807 - DNA, 23 bp, Homo sapiens
tgtgttacccctgctcccacgga

SEQ ID NO: 2808 - DNA, 26 bp, Homo sapiens
aaggtatgtgtgtgttgaggggtggg

SEQ ID NO: 2809 - DNA, 26 bp, Homo sapiens
atccgcccacctcggcctcccaaagt

SEQ ID NO: 2810 - DNA, 23 bp, Homo sapiens
ccagctacatcaaaacccaccac

SEQ ID NO: 2811 - DNA, 23 bp, Homo sapiens
tggcaaggaatccacaggttttc

SEQ ID NO: 2812 - DNA, 22 bp, Homo sapiens
tctccaaggcactcacaagtat

SEQ ID NO: 2813 - DNA, 22 bp, Homo sapiens
gaaacgttcatcaggagttgga

SEQ ID NO: 2814 - DNA, 26 bp, Homo sapiens
ttgtgagtgggaagcagtgggatttc

SEQ ID NO: 2815 - DNA, 26 bp, Homo sapiens
gtgttagggctggaaggaacttgttg

SEQ ID NO: 2816 - DNA, 25 bp, Homo sapiens
ctgcacactggagcagggaccttgg

SEQ ID NO: 2817 - DNA, 25 bp, Homo sapiens
accctcatctttggtgtgatgtttg

SEQ ID NO: 2818 - DNA, 26 bp, Homo sapiens
aaggaataggaggctgagtatggtgg

SEQ ID NO: 2819 - DNA, 26 bp, Homo sapiens
ggttcaactcctgaccttgccttcat

SEQ ID NO: 2820 - DNA, 23 bp, Homo sapiens
ggtaacagagtgtggactggtgc

SEQ ID NO: 2821 - DNA, 23 bp, Homo sapiens
tcaggcaggaccaagaccaccca

SEQ ID NO: 2822 - DNA, 23 bp, Homo sapiens
tggaagtgctgaactggagcccc

SEQ ID NO: 2823 - DNA, 23 bp, Homo sapiens
ccagtatcctgactcctgacagc

SEQ ID NO: 2824 - DNA, 23 bp, Homo sapiens
ttgtggaggaagatggctgccgc

SEQ ID NO: 2825 - DNA, 23 bp, Homo sapiens
tatggtggaatggacctgcctgc

SEQ ID NO: 2826 - DNA, 22 bp, Homo sapiens
tctccaaggcactcacaagtat

SEQ ID NO: 2827 - DNA, 22 bp, Homo sapiens
gaaacgttcatcaggagttgga

SEQ ID NO: 2828 - DNA, 23 bp, Homo sapiens
ccagtcttgaccacgcccaccta

SEQ ID NO: 2829 - DNA, 23 bp, Homo sapiens
ttgggcgagattccagcctacac

SEQ ID NO: 2830 - DNA, 24 bp, Homo sapiens
cacttcaattgaaaaaaaaaaaag

SEQ ID NO: 2831 - DNA, 24 bp, Homo sapiens
cgcccggctaagttttctgcagtt

SEQ ID NO: 2832 - DNA, 26 bp, Homo sapiens
gtcctaatccacaagggcatctttct

SEQ ID NO: 2833 - DNA, 26 bp, Homo sapiens
tcaaaggattcgctacttacagcagt

SEQ ID NO: 2834 - DNA, 23 bp, Homo sapiens
actcacaggtggcatttgatggc

SEQ ID NO: 2835 - DNA, 23 bp, Homo sapiens
tcaaaaatattcagagaaaatat

SEQ ID NO: 2836 - DNA, 26 bp, Homo sapiens
tgttatcgctcagccacttatcactg

SEQ ID NO: 2837 - DNA, 26 bp, Homo sapiens
ccactttcctttccttcccaccccag

SEQ ID NO: 2838 - DNA, 23 bp, Homo sapiens
acaatctgctctccaccaccagc

SEQ ID NO: 2839 - DNA, 23 bp, Homo sapiens
gccaaggtcgctggagatgtgag

SEQ ID NO: 2840 - DNA, 23 bp, Homo sapiens
ggtttgtgtggtgagacagtatt

SEQ ID NO: 2841 - DNA, 23 bp, Homo sapiens
aggctggtcttgaactcctaatc

SEQ ID NO: 2842 - DNA, 26 bp, Homo sapiens
gacaccagtgattggatttcaggttc

SEQ ID NO: 2843 - DNA, 26 bp, Homo sapiens
tcaaaggattcgctacttacagcagt

SEQ ID NO: 2844 - DNA, 26 bp, Homo sapiens
cccgtattcagagaccacccacttta

SEQ ID NO: 2845 - DNA, 24 bp, Homo sapiens
tcctgagggcttcattcaaacttg

SEQ ID NO: 2846 - DNA, 26 bp, Homo sapiens
ctgggagtggtagatgtaatgcctgt

SEQ ID NO: 2847 - DNA, 26 bp, Homo sapiens
gcctctaataagtgtggaatcctggg

SEQ ID NO: 2848 - DNA, 23 bp, Homo sapiens
aattcctctcctatgcacacatt

SEQ ID NO: 2849 - DNA, 23 bp, Homo sapiens
tcaagcgattcttctgcctcagc

SEQ ID NO: 2850 - DNA, 24 bp, Homo sapiens
tagcagagggctcctcccgctcat

SEQ ID NO: 2851 - DNA, 24 bp, Homo sapiens
tgggttgtattccacctgctattc

SEQ ID NO: 2852 - DNA, 22 bp, Homo sapiens
gtgggggcgagtgttgcgtgag

SEQ ID NO: 2853 - DNA, 23 bp, Homo sapiens
gcctctgccccactgctcaggag

SEQ ID NO: 2854 - DNA, 28 bp, Homo sapiens
ctggtgaggaaggaggaagagaggaggc

SEQ ID NO: 2855 - DNA, 28 bp, Homo sapiens
gaaaggtgggacaactggaagtgagagc

SEQ ID NO: 2856 - DNA, 28 bp, Homo sapiens
agagagcgggagacagagaaaggaaaag

SEQ ID NO: 2857 - DNA, 28 bp, Homo sapiens
ctcattcccagcaggagggtaaaggctt

SEQ ID NO: 2858 - DNA, 28 bp, Homo sapiens
ctggtgaggaaggaggaagagaggaggc

SEQ ID NO: 2859 - DNA, 22 bp, Homo sapiens
gttatggaccaaatgtctggcc

SEQ ID NO: 2860 - DNA, 28 bp, Homo sapiens
gaggaagtggaggcacaggcggctcaag

SEQ ID NO: 2861 - DNA, 28 bp, Homo sapiens
cctcacagccccattagaagtttatttc

SEQ ID NO: 2862 - DNA, 22 bp, Homo sapiens
aaaaaaataaaaaatagttaat

SEQ ID NO: 2863 - DNA, 23 bp, Homo sapiens
gccaggaagacgcaggagaccgc

SEQ ID NO: 2864 - DNA, 21 bp, Homo sapiens
caagcatgagtgacaaccaat

SEQ ID NO: 2865 - DNA, 28 bp, Homo sapiens
cagtgtcatagagtccaaagttgtaggc

SEQ ID NO: 2866 - DNA, 23 bp, Homo sapiens
tcagggattgtaaatacaccaat

SEQ ID NO: 2867 - DNA, 28 bp, Homo sapiens
cggtgacttcagttgggtggagggtggg

SEQ ID NO: 2868 - DNA, 28 bp, Homo sapiens
gaaaggtgggacaactggaagtgagagc

SEQ ID NO: 2869 - DNA, 27 bp, Homo sapiens
caggaggacggcagggctatggaccta

SEQ ID NO: 2870 - DNA, 22 bp, Homo sapiens
cctgctaggtgggagtcacctg

SEQ ID NO: 2871 - DNA, 24 bp, Homo sapiens
aacattcatgtcaaaactccctaa

SEQ ID NO: 2872 - DNA, 23 bp, Homo sapiens
cctctttggccagagccaaaacc

SEQ ID NO: 2873 - DNA, 22 bp, Homo sapiens
tttatgtctagctcagggattg

SEQ ID NO: 2874 - DNA, 21 bp, Homo sapiens
cagctcaggtggggtgtggtg

SEQ ID NO: 2875 - DNA, 24 bp, Homo sapiens
ggcatcagggtggcagtggcaggc

SEQ ID NO: 2876 - DNA, 22 bp, Homo sapiens
ggggccttggagaacctttatg

SEQ ID NO: 2877 - DNA, 28 bp, Homo sapiens
ggctggagtgggacattggctgggctgt

SEQ ID NO: 2878 - DNA, 28 bp, Homo sapiens
attttggtggttcataggcaggctgagc

SEQ ID NO: 2879 - DNA, 28 bp, Homo sapiens
agcctccctgcccatctgcccctctcat

SEQ ID NO: 2880 - DNA, 22 bp, Homo sapiens
aaagattcatgctctctcaagg

SEQ ID NO: 2881 - DNA, 24 bp, Homo sapiens
gaaaaaaataggtgtgtgaataat

SEQ ID NO: 2882 - DNA, 25 bp, Homo sapiens
gggtggggtgcctggatgcgtgagg

SEQ ID NO: 2883 - DNA, 28 bp, Homo sapiens
cctggatttgaactgccttgtccgtggg

SEQ ID NO: 2884 - DNA, 24 bp, Homo sapiens
gttctcttctgcacactggagcag

SEQ ID NO: 2885 - DNA, 28 bp, Homo sapiens
aaaggaataggaggctgagtatggtggc

SEQ ID NO: 2886 - DNA, 27 bp, Homo sapiens
gctgggtaacagagtgtggactggtgc

SEQ ID NO: 2887 - DNA, 23 bp, Homo sapiens
ggcaaccaggaggacggcagggc

SEQ ID NO: 2888 - DNA, 28 bp, Homo sapiens
ttgtggaggaagatggctgccgcctggg

SEQ ID NO: 2889 - DNA, 25 bp, Homo sapiens
gggtggggtgcctggatgcgtgagg

SEQ ID NO: 2890 - DNA, 23 bp, Homo sapiens
ccccagtcttgaccacgcccacc

SEQ ID NO: 2891 - DNA, 21 bp, Homo sapiens
cactgaattgttcagtttaaa

SEQ ID NO: 2892 - DNA, 28 bp, Homo sapiens
ctgataagaagaggattgtaggactgcc

SEQ ID NO: 2893 - DNA, 23 bp, Homo sapiens
actcaggagggaatgtaaccctc

SEQ ID NO: 2894 - DNA, 23 bp, Homo sapiens
gctcccgggccctgctaggtggg

SEQ ID NO: 2895 - DNA, 28 bp, Homo sapiens
cttcccgctctgctcttttgaggctggc

SEQ ID NO: 2896 - DNA, 22 bp, Homo sapiens
cagtattgaatccaacaaacgg

SEQ ID NO: 2897 - DNA, 28 bp, Homo sapiens
gggctcctctgtgtatctcctctgtctc

SEQ ID NO: 2898 - DNA, 28 bp, Homo sapiens
ccccgcaaaaggcttgaaggagcacccg

SEQ ID NO: 2899 - DNA, 28 bp, Homo sapiens
gtctccacggtctctgctgtctccttgc

SEQ ID NO: 2900 - DNA, 26 bp, Homo sapiens
attcccagcacaaatgattatcttcc

SEQ ID NO: 2901 - DNA, 24 bp, Homo sapiens
taggactaggatttgaacccaggc

SEQ ID NO: 2902 - DNA, 22 bp, Homo sapiens
ggtcatctagtcacatctcata

SEQ ID NO: 2903 - DNA, 40 bp, Homo sapiens
gtttgaaggtgtgtttgttcgaaaaagacttgcatttatg

SEQ ID NO: 2904 - DNA, 28 bp, Homo sapiens
gcaaacttcaggctgtattgttctcgtc

SEQ ID NO: 2905 - DNA, 36 bp, Homo sapiens
agtcccaggctttcgtctcacgttcgaataacaacc

SEQ ID NO: 2906 - DNA, 28 bp, Homo sapiens
ccagcgttacatctgttgtgccagcaaa

SEQ ID NO: 2907 - DNA, 37 bp, Homo sapiens
tcaggcaatccatgcttggagaaagtcgatagatggc

SEQ ID NO: 2908 - DNA, 28 bp, Homo sapiens
gccagtaaaccaaggcttattaggatac

SEQ ID NO: 2909 - DNA, 35 bp, Homo sapiens
agtggcagatagctgggaaaacattcgagccaagc

SEQ ID NO: 2910 - DNA, 24 bp, Homo sapiens
tcaggcagaggtggaccgagaacc

SEQ ID NO: 2911 - DNA, 39 bp, Homo sapiens
agttcagtctagaacataacagaaaatgtcttcgactct

SEQ ID NO: 2912 - DNA, 28 bp, Homo sapiens
gggacttgacagttcatctcattcattc

SEQ ID NO: 2913 - DNA, 32 bp, Homo sapiens
agggtagcccattgggtacctagctcgagttc

SEQ ID NO: 2914 - DNA, 28 bp, Homo sapiens
cctgttttggaatgcggtggcgtctggc

SEQ ID NO: 2915 - DNA, 35 bp, Homo sapiens
tggagaaagtcgatttccccattttggccgatggc

SEQ ID NO: 2916 - DNA, 22 bp, Homo sapiens
ttccgttttaggaaactagaaa

SEQ ID NO: 2917 - DNA, 40 bp, Homo sapiens
ggggccttgagaggtaattcgatcttttcaaagaaccaca

SEQ ID NO: 2918 - DNA, 25 bp, Homo sapiens
ctccccacactgccaagcccttacc

SEQ ID NO: 2919 - DNA, 32 bp, Homo sapiens
tgcatcaccacagagcacttcgacacacccct

SEQ ID NO: 2920 - DNA, 27 bp, Homo sapiens
tggtgtgtcctgggttttgtggagcac

SEQ ID NO: 2921 - DNA, 32 bp, Homo sapiens
agtgcagtcgaggcagtgggagttgaatcctc

SEQ ID NO: 2922 - DNA, 22 bp, Homo sapiens
ttctagtttggacaatgtatga

SEQ ID NO: 2923 - DNA, 40 bp, Homo sapiens
ttgcttgagctcaggagttcgaaaaagaaaagaaaagaac

SEQ ID NO: 2924 - DNA, 28 bp, Homo sapiens
ctactccgaacacaaagcatacataccc

SEQ ID NO: 2925 - DNA, 33 bp, Homo sapiens
tcgttgggtttggcggagggctcgataacttag

SEQ ID NO: 2926 - DNA, 21 bp, Homo sapiens
tttctcacctctagatatgtt

SEQ ID NO: 2927 - DNA, 40 bp, Homo sapiens
gatcacgaggccaggagttcgatagataatagatagaaat

SEQ ID NO: 2928 - DNA, 23 bp, Homo sapiens
gctgcctgctgttcccgtgccag

SEQ ID NO: 2929 - DNA, 32 bp, Homo sapiens
aaagacaaacgaggtcgagctgccgtgtgtcc

SEQ ID NO: 2930 - DNA, 23 bp, Homo sapiens
ttttcaagcagggcttcagcttt

SEQ ID NO: 2931 - DNA, 40 bp, Homo sapiens
ggtttgtgaatgcaccaatcgaggtgaactgtggcaatga

SEQ ID NO: 2932 - DNA, 24 bp, Homo sapiens
tacccgcaagtgtctgtgcctggc

SEQ ID NO: 2933 - DNA, 35 bp, Homo sapiens
tgccgcctaaggttgttgatgtagctctcgaacgt

SEQ ID NO: 2934 - DNA, 28 bp, Homo sapiens
cagaccagtattccgacagcaggcttgc

SEQ ID NO: 2935 - DNA, 37 bp, Homo sapiens
tgagtggcagatagctgggaaaacattcgataacccc

SEQ ID NO: 2936 - DNA, 28 bp, Homo sapiens
ggagggaggaataggcagagcacagcag

SEQ ID NO: 2937 - DNA, 37 bp, Homo sapiens
agttcaaatcaagctaggaggcatccgtgtcgaaaga

SEQ ID NO: 2938 - DNA, 22 bp, Homo sapiens
tacattctacaaggtgaaagac

SEQ ID NO: 2939 - DNA, 40 bp, Homo sapiens
gggtacgagggggagacctcgacaccaacatatatctgtg

SEQ ID NO: 2940 - DNA, 24 bp, Homo sapiens
tgagagatgcttccctgttgtatt

SEQ ID NO: 2941 - DNA, 40 bp, Homo sapiens
gaatgcaccatcaatgaatcgattctgatgacatggttgt

SEQ ID NO: 2942 - DNA, 23 bp, Homo sapiens
agctaccgcgcccagcatccatt

SEQ ID NO: 2943 - DNA, 40 bp, Homo sapiens
agctcagtcatgtgaacctcgaaaaagaaagaaacatttc

SEQ ID NO: 2944 - DNA, 22 bp, Homo sapiens
tttctaagtgagatgtggaaat

SEQ ID NO: 2945 - DNA, 40 bp, Homo sapiens
ggtttgtgaatgcaccaatcgaacagtccaaataaagtct

SEQ ID NO: 2946 - DNA, 21 bp, Homo sapiens
ggaactcaatttttattcagc

SEQ ID NO: 2947 - DNA, 40 bp, Homo sapiens
cgcttagggatgcagacgtcgaatagtccactgttactgg

SEQ ID NO: 2948 - DNA, 28 bp, Homo sapiens
gaggttgagtgacaaatgctgtgctctc

SEQ ID NO: 2949 - DNA, 37 bp, Homo sapiens
agctcaggagttcgatatctgtttacttaaccacaaa

SEQ ID NO: 2950 - DNA, 22 bp, Homo sapiens
actacagaaacataatgactaa

SEQ ID NO: 2951 - DNA, 40 bp, Homo sapiens
ggtttgtgaatgcaccaatcgatattttacttaaccatag

SEQ ID NO: 2952 - DNA, 25 bp, Homo sapiens
ccagcacccagaggcaaactcaccc

SEQ ID NO: 2953 - DNA, 33 bp, Homo sapiens
tctgcccaacaagagagagctgggatcgaatgc

SEQ ID NO: 2954 - DNA, 23 bp, Homo sapiens
gccccaggtgccagcagggtgac

SEQ ID NO: 2955 - DNA, 38 bp, Homo sapiens
actatacatcctccaaggtgatgataacttcgagagtg

SEQ ID NO: 2956 - DNA, 25 bp, Homo sapiens
gactgtgttacccctgctcccacgg

SEQ ID NO: 2957 - DNA, 32 bp, Homo sapiens
aggcacgggtaccaagactacttcgatgctgc

SEQ ID NO: 2958 - DNA, 22 bp, Homo sapiens
tggtcaggctggtctccaactc

SEQ ID NO: 2959 - DNA, 40 bp, Homo sapiens
tcacttgaggtcaagagttcgagtcaaagtgatgctccat

SEQ ID NO: 2960 - DNA, 24 bp, Homo sapiens
tttaggaatgtcaaccattttgcc

SEQ ID NO: 2961 - DNA, 40 bp, Homo sapiens
aaaacaaaaatttaaaactcgagggggcgtagttctgccg

SEQ ID NO: 2962 - DNA, 25 bp, Homo sapiens
ctctctctccctctctttccctctc

SEQ ID NO: 2963 - DNA, 40 bp, Homo sapiens; identical to SEQ ID NO: 1903

SEQ ID NO: 2964 - DNA, 28 bp, Homo sapiens
gacagtgctttctctaaggctgggaacg

SEQ ID NO: 2965 - DNA, 32 bp, Homo sapiens
tcctccgtgggtcaggaagggtcgacaatcat

SEQ ID NO: 2966 - DNA, 24 bp, Homo sapiens
tcgggacagagtggagtcttcccc

SEQ ID NO: 2967 - DNA, 40 bp, Homo sapiens
agctcagtcatgtgaacctcgacagtaggtatttttggaa

SEQ ID NO: 2968 - DNA, 24 bp, Homo sapiens
gccctcttccctgctgcccctgtg

SEQ ID NO: 2969 - DNA, 36 bp, Homo sapiens
aggaggatcacttgaggccaagaattcgattcgtcc

SEQ ID NO: 2970 - DNA, 28 bp, Homo sapiens
gagtcaggcaggaccaagaccacccagc

SEQ ID NO: 2971 - DNA, 39 bp, Homo sapiens
accttgagctagatacagagtgtcgaaatcagcttgagg

SEQ ID NO: 2972 - DNA, 28 bp, Homo sapiens
cattctgctgttgatgggcattgggttg

SEQ ID NO: 2973 - DNA, 33 bp, Homo sapiens
aggaggcatccgtgtcgaattttcatggcagcc

SEQ ID NO: 2974 - DNA, 24 bp, Homo sapiens
gtggaatggacctgcctgcctggc

SEQ ID NO: 2975 - DNA, 31 bp, Homo sapiens
tttagcgtgaggcgctccaggtcgagtggtt

SEQ ID NO: 2976 - DNA, 25 bp, Homo sapiens
ctctctctccctctctttccctctc

SEQ ID NO: 2977 - DNA, 40 bp, Homo sapiens; identical to SEQ ID NO: 1903

SEQ ID NO: 2978 - DNA, 28 bp, Homo sapiens
gcttgggcgagattccagcctacacggg

SEQ ID NO: 2979 - DNA, 34 bp, Homo sapiens
agagcttaagttcgagcagtgtggaaccttcggg

SEQ ID NO: 2980 - DNA, 21 bp, Homo sapiens
ggcatttcgccacgttggcca

SEQ ID NO: 2981 - DNA, 40 bp, Homo sapiens
gacttgagcttcaggagttcgagaccagtttggccaacaa

SEQ ID NO: 2982 - DNA, 25 bp, Homo sapiens
ctccccacactgccaagcccttacc

SEQ ID NO: 2983 - DNA, 32 bp, Homo sapiens
ttgcaggtcgaagtgctctgtggtgatgcagc

SEQ ID NO: 2984 - DNA, 23 bp, Homo sapiens
agaaaatatccacagagttccaa

SEQ ID NO: 2985 - DNA, 40 bp, Homo sapiens
aggtgctatgcttatgactcgagatctccactcagatacc

SEQ ID NO: 2986 - DNA, 23 bp, Homo sapiens
cttgctgggggcttgacttctct

SEQ ID NO: 2987 - DNA, 40 bp, Homo sapiens
gggtacgagggggagacctcgatgagcagcatgttattta

SEQ ID NO: 2988 - DNA, 28 bp, Homo sapiens
catcaccccagcctttgctatcacgggc

SEQ ID NO: 2989 - DNA, 32 bp, Homo sapiens
aagtcccaggctttcgtctcacgttcgacagc

SEQ ID NO: 2990 - DNA, 22 bp, Homo sapiens
ccacctgcctcggcctcccaaa

SEQ ID NO: 2991 - DNA, 40 bp, Homo sapiens
cataagactaaggttctgtcgaaccacctttatttactgt

SEQ ID NO: 2992 - DNA, 26 bp, Homo sapiens
gctccccacactgccaagcccttacc

SEQ ID NO: 2993 - DNA, 34 bp, Homo sapiens
ttcaactcgaagtgctctgtggtgatgcagcagg

SEQ ID NO: 2994 - DNA, 25 bp, Homo sapiens
gggagtggcaggtccaagactaccc

SEQ ID NO: 2995 - DNA, 36 bp, Homo sapiens
agctcaggagttcgaaacccaacataaatgcaaata

SEQ ID NO: 2996 - DNA, 28 bp, Homo sapiens
gagcctctaataagtgtggaatcctggg

SEQ ID NO: 2997 - DNA, 33 bp, Homo sapiens
aggtggatcacttgaggccacgagttcgagtac

SEQ ID NO: 2998 - DNA, 26 bp, Homo sapiens
tgagatggagtttagcttttgttgcc

SEQ ID NO: 2999 - DNA, 40 bp, Homo sapiens
tcacctaagctcaggagttcgagactagcttggccaacat

SEQ ID NO: 3000 - DNA, 24 bp, Homo sapiens
gtgccatggattctgtccttcgga

SEQ ID NO: 3001 - DNA, 40 bp, Homo sapiens
gcccattgggtacctagctcgaatgcaggctgttgttgga

SEQ ID NO: 3002 - DNA, 40 bp, Homo sapiens
cataaatgcaagtctttttcgaacaaacacaccttcaaac

SEQ ID NO: 3003 - DNA, 36 bp, Homo sapiens
tggttgttattcgaacgtgagacgaaagcctgggac

SEQ ID NO: 3004 - DNA, 37 bp, Homo sapiens
tctatcgactttctccaagcatggattgcctgagctc

SEQ ID NO: 3005 - DNA, 35 bp, Homo sapiens
tggctcgaatgttttcccagctatctgccactcac

SEQ ID NO: 3006 - DNA, 39 bp, Homo sapiens
agagtcgaagacattttctgttatgttctagactgaact

SEQ ID NO: 3007 - DNA, 32 bp, Homo sapiens
tggaactcgagctaggtacccaatgggctacc

SEQ ID NO: 3008 - DNA, 35 bp, Homo sapiens
gccatcggccaaaatggggaaatcgactttctcca

SEQ ID NO: 3009 - DNA, 40 bp, Homo sapiens
tgtggttctttgaaaagatcgaattacctctcaaggcccc

SEQ ID NO: 3010 - DNA, 32 bp, Homo sapiens
aggggtgtgtcgaagtgctctgtggtgatgca

SEQ ID NO: 3011 - DNA, 32 bp, Homo sapiens
agaggattcaactcccactgcctcgactgcac

SEQ ID NO: 3012 - DNA, 40 bp, Homo sapiens
gttcttttcttttctttttcgaactcctgagctcaagcaa

SEQ ID NO: 3013 - DNA, 33 bp, Homo sapiens
agttatcgagccctccgccaaacccaacgattc

SEQ ID NO: 3014 - DNA, 40 bp, Homo sapiens
atttctatctattatctatcgaactcctggcctcgtgatc

SEQ ID NO: 3015 - DNA, 32 bp, Homo sapiens
tatgggacacacggcagctcgacctcgtttgt

SEQ ID NO: 3016 - DNA, 40 bp, Homo sapiens
tcattgccacagttcacctcgattggtgcattcacaaacc

SEQ ID NO: 3017 - DNA, 34 bp, Homo sapiens
acgttcgagagctacatcaacaaccttaggcggc

SEQ ID NO: 3018 - DNA, 37 bp, Homo sapiens
ggggttatcgaatgttttcccagctatctgccactca

SEQ ID NO: 3019 - DNA, 36 bp, Homo sapiens
tctttcgacacggatgcctcctagcttgatttgaac

SEQ ID NO: 3020 - DNA, 40 bp, Homo sapiens
cacagatatatgttggtgtcgaggtctccccctcgtaccc

SEQ ID NO: 3021 - DNA, 40 bp, Homo sapiens
acaaccatgtcatcagaatcgattcattgatggtgcattc

SEQ ID NO: 3022 - DNA, 40 bp, Homo sapiens
gaaatgtttctttctttttcgaggttcacatgactgagct

SEQ ID NO: 3023 - DNA, 40 bp, Homo sapiens
agactttatttggactgttcgattggtgcattcacaaacc

SEQ ID NO: 3024 - DNA, 40 bp, Homo sapiens
ccagtaacagtggactattcgacgtctgcatccctaagcg

SEQ ID NO: 3025 - DNA, 39 bp, Homo sapiens
tctttgtggttaagtaaacagatatcgaactcctgagct

SEQ ID NO: 3026 - DNA, 40 bp, Homo sapiens
ctatggttaagtaaaatatcgattggtgcattcacaaacc

SEQ ID NO: 3027 - DNA, 33 bp, Homo sapiens
tgcattcgatcccagctctctcttgttgggcag

SEQ ID NO: 3028 - DNA, 38 bp, Homo sapiens
acactctcgaagttatcatcaccttggaggatgtatag

SEQ ID NO: 3029 - DNA, 32 bp, Homo sapiens
tgcagcatcgaagtagtcttggtacccgtgcc

SEQ ID NO: 3030 - DNA, 40 bp, Homo sapiens
atggagcatcactttgactcgaactcttgacctcaagtga

SEQ ID NO: 3031 - DNA, 40 bp, Homo sapiens
cggcagaactacgccccctcgagttttaaatttttgtttt

SEQ ID NO: 3032 - DNA, 40 bp, Homo sapiens; identical to SEQ ID NO: 1977

SEQ ID NO: 3033 - DNA, 32 bp, Homo sapiens
atgattgtcgacccttcctgacccacggagga

SEQ ID NO: 3034 - DNA, 40 bp, Homo sapiens
ttccaaaaatacctactgtcgaggttcacatgactgagct

SEQ ID NO: 3035 - DNA, 36 bp, Homo sapiens
acgaatcgaattcttggcctcaagtgatcctcctgc

SEQ ID NO: 3036 - DNA, 35 bp, Homo sapiens
agtccccaagacctcaagctgatttcgacactctg

SEQ ID NO: 3037 - DNA, 33 bp, Homo sapiens
agggctgccatgaaaattcgacacggatgcctc

SEQ ID NO: 3038 - DNA, 31 bp, Homo sapiens
aaccactcgacctggagcgcctcacgctaaa

SEQ ID NO: 3039 - DNA, 40 bp, Homo sapiens; identical to SEQ ID NO: 1977

SEQ ID NO: 3040 - DNA, 34 bp, Homo sapiens
tcccgaaggttccacactgctcgaacttaagctc

SEQ ID NO: 3041 - DNA, 40 bp, Homo sapiens
ttgttggccaaactggtctcgaactcctgaagctcaagtc

SEQ ID NO: 3042 - DNA, 32 bp, Homo sapiens
tgctgcatcaccacagagcacttcgacctgca

SEQ ID NO: 3043 - DNA, 40 bp, Homo sapiens
ggtatctgagtggagatctcgagtcataagcatagcacct

SEQ ID NO: 3044 - DNA, 40 bp, Homo sapiens
taaataacatgctgctcatcgaggtctccccctcgtaccc

SEQ ID NO: 3045 - DNA, 32 bp, Homo sapiens
taggctgtcgaacgtgagacgaaagcctggga

SEQ ID NO: 3046 - DNA, 40 bp, Homo sapiens
acagtaaataaaggtggttcgacagaaccttagtcttatg

SEQ ID NO: 3047 - DNA, 35 bp, Homo sapiens
acctgctgcatcaccacagagcacttcgagttgaa

SEQ ID NO: 3048 - DNA, 36 bp, Homo sapiens
atttgcatttatgttgggtttcgaactcctgagctc

SEQ ID NO: 3049 - DNA, 32 bp, Homo sapiens
agtactcgaactcgtggcctcaagtgatccac

SEQ ID NO: 3050 - DNA, 40 bp, Homo sapiens
atgttggccaagctagtctcgaactcctgagcttaggtga

SEQ ID NO: 3051 - DNA, 40 bp, Homo sapiens
tccaacaacagcctgcattcgagctaggtacccaatgggc

SEQ ID NO: 3052 - DNA, 60 bp, Homo sapiens
ggcaggaggattgcttgagttcaggagttcgaacaagatattaagtattagaagaacaaa

SEQ ID NO: 3053 - DNA, 60 bp, Homo sapiens
caccaccagagtagctagatacagagtgtcgattgtcatgttttcactgacaaatatgtc

SEQ ID NO: 3054 - DNA, 60 bp, Homo sapiens
ggcaggcagatcacctgaggtttggagttcgaatgtttctctaatgcatgtcttctttgg

SEQ ID NO: 3055 - DNA, 60 bp, Homo sapiens
gtggtggtacaggtgctatgcttatgactcgagacatgtggagtcaccccatttgcagtg

SEQ ID NO: 3056 - DNA, 60 bp, Homo sapiens
gtgtttcctagttagctcagagcctcagtcgaaggaagcttaggaatatagcttatgtgt

SEQ ID NO: 3057 - DNA, 60 bp, Homo sapiens
agcaggcagatcacttgaggtcagaaattcgacctccttgctcaactgtcttttatccac

SEQ ID NO: 3058 - DNA, 60 bp, Homo sapiens
aggagctggggtgtatgtcaacaatttctcgaactcccaggttcaaacgatcctctcacc

SEQ ID NO: 3059 - DNA, 60 bp, Homo sapiens
aaggagctggtcggtattcccctcgctctcgagtaaaagtggcagaagatggttccatta

SEQ ID NO: 3060 - DNA, 60 bp, Homo sapiens
agggagtagaggaatcgggagccagtgttcgaataatacatctcttcaatattcctgatt

SEQ ID NO: 3061 - DNA, 60 bp, Homo sapiens
agtaatgtcctttagagctgttttctcctcgaagggctggttgtactgcggaggaggaag

SEQ ID NO: 3062 - DNA, 60 bp, Homo sapiens
aaggcaggcagatcactaggtcaggagttcgaaacccacactgcaagtatcaacaggcaa

SEQ ID NO: 3063 - DNA, 60 bp, Homo sapiens
gaggcaggcggatcacgaggtcaatagatcgataggaaacaaacatttgatccgttagta

SEQ ID NO: 3064 - DNA, 60 bp, Homo sapiens
agtggcatgatcatagttcactgcagcctcgatacacattttcaaagtgcttttcaaaaa

SEQ ID NO: 3065 - DNA, 60 bp, Homo sapiens
ggcaggagaattgcttgagcccagaagttcgagttttccccttgctcctaaatcccagtc

SEQ ID NO: 3066 - DNA, 60 bp, Homo sapiens
ggcaggcagatcacttgaggtaaggagttcgaaaattagcagaaaaaagcaagtaaagat

SEQ ID NO: 3067 - DNA, 60 bp, Homo sapiens
ggcaggcggatcacctgagttcaggagttcgagatgacagctaaacataaaatgaaccaa

SEQ ID NO: 3068 - DNA, 60 bp, Homo sapiens
gtgtttcctagttagctcagagcctcagtcgagtcatatttgaatgtctactacatcatc

SEQ ID NO: 3069 - DNA, 60 bp, Homo sapiens
aggcaggcagatcacttgaggttagggttcgaccagcagcaaagacagcaagtattccag

SEQ ID NO: 3070 - DNA, 60 bp, Homo sapiens
gcagtgagccgagatcatgccattatactcgaaacaggaaaaaaagaagtctgcttctga

SEQ ID NO: 3071 - DNA, 60 bp, Homo sapiens
ggcaggaggatcacttgagcctaggaagtcgatgtttcttgaattgttttatttgtgttg

SEQ ID NO: 3072 - DNA, 60 bp, Homo sapiens
aggcaagagaatcacttgaacccgggagtcgagatcagcctgaccaacatgaaaccccgt

SEQ ID NO: 3073 - DNA, 60 bp, Homo sapiens
agcaggcagatcacttgaggtcagaaattcgaaacgaagggctaagcccccctcgcccgg

SEQ ID NO: 3074 - DNA, 60 bp, Homo sapiens
agcaggaggatcacttgaggccagggattcgatcctcaagctaagaataaaaacaccaag

SEQ ID NO: 3075 - DNA, 60 bp, Homo sapiens
caaaggcaaatgcgtcctgtcactctcctcgaggttatatttattctaattattattatc

SEQ ID NO: 3076 - DNA, 60 bp, Homo sapiens
gttaaagtgattctcctgcctcagactctcgatacataaggacttaggaaaaatgataaa

SEQ ID NO: 3077 - DNA, 60 bp, Homo sapiens
gagggagggagataagcttaagaagagttcgagacacttattttggagatggcatcttgc

SEQ ID NO: 3078 - DNA, 60 bp, Homo sapiens
ggcaggtggatcacctgaggtcaggaattcgaaaaaaaaaaaaaaaagaaataaagaaaa

SEQ ID NO: 3079 - DNA, 60 bp, Homo sapiens
gtgtttcctagttagctcagagcctcagtcgaatacacactcattgaaggcccactatgt

SEQ ID NO: 3080 - DNA, 60 bp, Homo sapiens
gaggtgggcagatcacgaggacaggagatcgagcctgtaaacaaaaagcaaacaactact

SEQ ID NO: 3081 - DNA, 60 bp, Homo sapiens
cccacacaatttaaatctgtcttgtacatcgaggtgagagtgagctgagatggcgccact

SEQ ID NO: 3082 - DNA, 60 bp, Homo sapiens
agcaggcagatcatctgaggttgggagttcgaagacacaatttcttgttagtgactctat

SEQ ID NO: 3083 - DNA, 60 bp, Homo sapiens
taaagctatgcataagactaaggttctgtcgatacatatttgtttaatgaacaaacatcc

SEQ ID NO: 3084 - DNA, 60 bp, Homo sapiens
aggtttctccatgttggtcagtctggtctcgaagtcactcctaactcatagaaaatgtgt

SEQ ID NO: 3085 - DNA, 60 bp, Homo sapiens
aggcagcttgtctaggtaaagttccgggtcgaataggcctaagtttgtggaatgatagat

SEQ ID NO: 3086 - DNA, 60 bp, Homo sapiens
aaggcaggtggatcacgaggtcagcagatcgatacataattttacaaaaccaaacacatt

SEQ ID NO: 3087 - DNA, 60 bp, Homo sapiens
ggcaggcagatcacctgaggccaggaattcgaaatactatgctaagattcaacattactc

SEQ ID NO: 3088 - DNA, 60 bp, Homo sapiens
ctgggggtaccgtgatcgggggtaggcctcgaaaggtcattataaggatggagtgagatg

SEQ ID NO: 3089 - DNA, 60 bp, Homo sapiens
tgacacagtcaaacattaacttggtgtatcgaagggctggttgtactgcggaggaggaag

SEQ ID NO: 3090 - DNA, 60 bp, Homo sapiens
tttgcaggtgtgtggttaacactatccttcgacctgtactatctgatggacctctcctac

SEQ ID NO: 3091 - DNA, 60 bp, Homo sapiens
agtggtgccatcatagctcactgcagcctcgaataaaagaaaataaaaagacatgattaa

SEQ ID NO: 3092 - DNA, 60 bp, Homo sapiens
ccaagtgcaccacgtgaacaaaacaatctcgattgggaataactcaccttttttatgcca

SEQ ID NO: 3093 - DNA, 60 bp, Homo sapiens
aggcaggtggatcacttgagctcaggattcgaaaacattaactgaaagaaattggacaca

SEQ ID NO: 3094 - DNA, 60 bp, Homo sapiens
tcagacagatcaggaaaacggaatgacatcgagaaagtggaactggtaagcataggggtc

SEQ ID NO: 3095 - DNA, 60 bp, Homo sapiens
ctacccagcccctggtgggcaggtgagttcgagctgccgtgtgtcccatagccaccagct

SEQ ID NO: 3096 - DNA, 60 bp, Homo sapiens
gtggtcgtacaggtgctatgcttatgactcgagcttgcactccggtacccgacccggcgc

SEQ ID NO: 3097 - DNA, 60 bp, Homo sapiens
gaggctgaggcaagagaattgcttgaactcgattcagcatatgaaggggaagtgatacag

SEQ ID NO: 3098 - DNA, 60 bp, Homo sapiens
ggtgggaggattgcttgagcctgaaaggtcgaaaaataaaaatgaaaataaaaaaaaggc

SEQ ID NO: 3099 - DNA, 60 bp, Homo sapiens
ggcaggaggatcgcttgaacccaggagttcgaaataaaaaaaaaaaaaaaaagaaaagaa

SEQ ID NO: 3100 - DNA, 60 bp, Homo sapiens
ctccagccgcaagaagtacagcagcacctcgatctcactagtaattaggaaaatgaaaat

SEQ ID NO: 3101 - DNA, 60 bp, Homo sapiens
cagcagtgccagcctaccggtgctgcactcgacagaaagcttaagggcaaagatggtttt

SEQ ID NO: 3102 - DNA, 26 bp, Homo sapiens
gctgctcaacaagacacaatgtaagg

SEQ ID NO: 3103 - DNA, 26 bp, Homo sapiens
caccattctttctacctggaatggct

SEQ ID NO: 3104 - DNA, 26 bp, Homo sapiens
ccacccacatcctgctgattggtcca

SEQ ID NO: 3105 - DNA, 26 bp, Homo sapiens
tacacttaagaatttttgtatacatg

SEQ ID NO: 3106 - DNA, 21 bp, Homo sapiens
ctatcggagactctaaatgta

SEQ ID NO: 3107 - DNA, 21 bp, Homo sapiens
acctaaaggctctttaacgta

SEQ ID NO: 3108 - DNA, 23 bp, Homo sapiens
gtaaggaaggagaagatgaacac

SEQ ID NO: 3109 - DNA, 23 bp, Homo sapiens
tcttcctgttcttcctggaggag

SEQ ID NO: 3110 - DNA, 21 bp, Homo sapiens
tggctcgtggctctcactgca

SEQ ID NO: 3111 - DNA, 21 bp, Homo sapiens
tgccttctgtcatacattttg

SEQ ID NO: 3112 - DNA, 24 bp, Homo sapiens
cacagcaggtgggaaaatgtagcc

SEQ ID NO: 3113 - DNA, 23 bp, Homo sapiens
ctggcacatagcaggtggctcgt

SEQ ID NO: 3114 - DNA, 21 bp, Homo sapiens
acacacaagctgacacagcaa

SEQ ID NO: 3115 - DNA, 21 bp, Homo sapiens
ggcaacatagtgagaaccccg

SEQ ID NO: 3116 - DNA, 26 bp, Homo sapiens
atctactctgccgtggatgatgcctc

SEQ ID NO: 3117 - DNA, 26 bp, Homo sapiens
cagcctgactcaatgaccagtaaaga

SEQ ID NO: 3118 - DNA, 26 bp, Homo sapiens
tagagacaaagtagaatggtgggtcc

SEQ ID NO: 3119 - DNA, 26 bp, Homo sapiens
gcggatggatggcttgagtctgaaag

SEQ ID NO: 3120 - DNA, 25 bp, Homo sapiens
ccacggatagctgcagacatccaga

SEQ ID NO: 3121 - DNA, 25 bp, Homo sapiens
ttggtggggctgggggtgctcctga

SEQ ID NO: 3122 - DNA, 25 bp, Homo sapiens
aaataagtaaagacgtaaaccagca

SEQ ID NO: 3123 - DNA, 25 bp, Homo sapiens
tatgtaacaatacatgaactttgag

SEQ ID NO: 3124 - DNA, 26 bp, Homo sapiens
tggtgaaaacctatctcggctgggca

SEQ ID NO: 3125 - DNA, 26 bp, Homo sapiens
gaagtgtgatgtgcctctctttctgt

SEQ ID NO: 3126 - DNA, 26 bp, Homo sapiens
ccagcagacaaatcccagttcaatgc

SEQ ID NO: 3127 - DNA, 26 bp, Homo sapiens
tcacaaagcaggagggagaacaggtt

SEQ ID NO: 3128 - DNA, 26 bp, Homo sapiens
agaaacagtcctgctaacatcacccc

SEQ ID NO: 3129 - DNA, 26 bp, Homo sapiens
ctttcattcaacacccacggaggagt

SEQ ID NO: 3130 - DNA, 25 bp, Homo sapiens
cattctgcaacagccccacacagaa

SEQ ID NO: 3131 - DNA, 25 bp, Homo sapiens
cccaaaatgctgggattacaggcgt

SEQ ID NO: 3132 - DNA, 25 bp, Homo sapiens
aagaaaaagaaaaaaagacacaaat

SEQ ID NO: 3133 - DNA, 25 bp, Homo sapiens
attcatgtaacaacttaattaggga

SEQ ID NO: 3134 - DNA, 26 bp, Homo sapiens
cagactggggtgaagagttcagtcac

SEQ ID NO: 3135 - DNA, 26 bp, Homo sapiens
aggggctgaaatgatggatacctgtg

SEQ ID NO: 3136 - DNA, 26 bp, Homo sapiens
actccgtctcaaaaaaaaagaaaaaa

SEQ ID NO: 3137 - DNA, 26 bp, Homo sapiens
taaaataactgtatttaagggctact

SEQ ID NO: 3138 - DNA, 23 bp, Homo sapiens
ttgggaggctgaggcaggagaat

SEQ ID NO: 3139 - DNA, 26 bp, Homo sapiens
gggagtgagaaagaggaagtatcaaa

SEQ ID NO: 3140 - DNA, 23 bp, Homo sapiens
ggagttcaagaccagcctggagc

SEQ ID NO: 3141 - DNA, 23 bp, Homo sapiens
ttacggttaccgctgccccagcc

SEQ ID NO: 3142 - DNA, 26 bp, Homo sapiens
tacacaggttggaaagacgaagaaaa

SEQ ID NO: 3143 - DNA, 24 bp, Homo sapiens
gagtagttgggattacaggcgggt

SEQ ID NO: 3144 - DNA, 23 bp, Homo sapiens
ctcacgcctgtaatcccagcact

SEQ ID NO: 3145 - DNA, 23 bp, Homo sapiens
cccttccaacccaatcaccccac

SEQ ID NO: 3146 - DNA, 26 bp, Homo sapiens
aaattacaatatacaaattacataca

SEQ ID NO: 3147 - DNA, 26 bp, Homo sapiens
tgtgcctgctgctatgcccaaatgcc

SEQ ID NO: 3148 - DNA, 25 bp, Homo sapiens
ccccaccaccaccctcagccaagcc

SEQ ID NO: 3149 - DNA, 25 bp, Homo sapiens
ggcagctagcaagagtgggggagac

SEQ ID NO: 3150 - DNA, 21 bp, Homo sapiens
actttaataacatctggtaat

SEQ ID NO: 3151 - DNA, 21 bp, Homo sapiens
tccttctctgtaaagaggaac

SEQ ID NO: 3152 - DNA, 24 bp, Homo sapiens
gcaaacggagagtgaaaaagtcat

SEQ ID NO: 3153 - DNA, 24 bp, Homo sapiens
aagtgccctattttcatatcccct

SEQ ID NO: 3154 - DNA, 24 bp, Homo sapiens
ggaagaatttgagggttaaggaga

SEQ ID NO: 3155 - DNA, 24 bp, Homo sapiens
cctcactatttgatcccaggcctg

SEQ ID NO: 3156 - DNA, 26 bp, Homo sapiens
aacgggaaagccacagactggggtga

SEQ ID NO: 3157 - DNA, 26 bp, Homo sapiens
taaccctaggagactccgtgtactac

SEQ ID NO: 3158 - DNA, 26 bp, Homo sapiens
ttgaaatgtgagaaggttgggctggg

SEQ ID NO: 3159 - DNA, 25 bp, Homo sapiens
agaacaagaagtctgaggagagtag

SEQ ID NO: 3160 - DNA, 23 bp, Homo sapiens
gcccagcctctcctcatcagtga

SEQ ID NO: 3161 - DNA, 23 bp, Homo sapiens
gacaccctcaagcacacactcag

SEQ ID NO: 3162 - DNA, 26 bp, Homo sapiens
tattccgagaccattgtgtgtgagtc

SEQ ID NO: 3163 - DNA, 26 bp, Homo sapiens
agggcattctcctaataactgtgatg

SEQ ID NO: 3164 - DNA, 22 bp, Homo sapiens
gatagtgtggcccaagatgcaa

SEQ ID NO: 3165 - DNA, 22 bp, Homo sapiens
cgactcaggtgagctgtttgat

SEQ ID NO: 3166 - DNA, 21 bp, Homo sapiens
gtatgcaatggcacgatctcg

SEQ ID NO: 3167 - DNA, 21 bp, Homo sapiens
aaatgtttggatggttaagtc

SEQ ID NO: 3168 - DNA, 26 bp, Homo sapiens
catagttctcatagaccagggtttgc

SEQ ID NO: 3169 - DNA, 26 bp, Homo sapiens
aagttgtgccaaagccacctgtgcca

SEQ ID NO: 3170 - DNA, 26 bp, Homo sapiens
cccttttctcagcacagcagtggaat

SEQ ID NO: 3171 - DNA, 26 bp, Homo sapiens
aaagacctgatgtatttcctaccagc

SEQ ID NO: 3172 - DNA, 24 bp, Homo sapiens
atgttatcctcttatattccacca

SEQ ID NO: 3173 - DNA, 24 bp, Homo sapiens
gggaaggctctgttacttgtagat

SEQ ID NO: 3174 - DNA, 23 bp, Homo sapiens
ttttctgccaggctctgctgcga

SEQ ID NO: 3175 - DNA, 26 bp, Homo sapiens
gaagagttcacaatacactaagggaa

SEQ ID NO: 3176 - DNA, 21 bp, Homo sapiens
ataaagctccaccttctttat

SEQ ID NO: 3177 - DNA, 21 bp, Homo sapiens
ggggctgggggtgctcctgag

SEQ ID NO: 3178 - DNA, 23 bp, Homo sapiens
gggctggagttgaggcttggttt

SEQ ID NO: 3179 - DNA, 23 bp, Homo sapiens
cagggtctgggaaaggactgggt

SEQ ID NO: 3180 - DNA, 23 bp, Homo sapiens
aatcctcctgcctcagcctcccg

SEQ ID NO: 3181 - DNA, 25 bp, Homo sapiens
tttcttcttccatccccatcattgc

SEQ ID NO: 3182 - DNA, 23 bp, Homo sapiens
tccccacacagacctcctctcct

SEQ ID NO: 3183 - DNA, 23 bp, Homo sapiens
gaaccccaagatggtgaagaggc

SEQ ID NO: 3184 - DNA, 23 bp, Homo sapiens
cccaggcttgtcttcaacttctg

SEQ ID NO: 3185 - DNA, 23 bp, Homo sapiens
ctcctagaaaccattaatctact

SEQ ID NO: 3186 - DNA, 23 bp, Homo sapiens
ggacaaggaggcagagtgacacg

SEQ ID NO: 3187 - DNA, 23 bp, Homo sapiens
ctgtcttcactgctcctctcagg

SEQ ID NO: 3188 - DNA, 23 bp, Homo sapiens
gggcaccctctcctgtttcactc

SEQ ID NO: 3189 - DNA, 23 bp, Homo sapiens
tgggcagtggaaataaggcaggc

SEQ ID NO: 3190 - DNA, 24 bp, Homo sapiens
gcttcattctgtgtaaggaaggag

SEQ ID NO: 3191 - DNA, 24 bp, Homo sapiens
ccccaaacacggaggccccaaggg

SEQ ID NO: 3192 - DNA, 23 bp, Homo sapiens
gctcacacctgtaatcccagcac

SEQ ID NO: 3193 - DNA, 23 bp, Homo sapiens
tcatttttaagcttctaagtaac

SEQ ID NO: 3194 - DNA, 25 bp, Homo sapiens
taatcccagcactttgggaggctga

SEQ ID NO: 3195 - DNA, 25 bp, Homo sapiens
taatataatgttatattaaataaat

SEQ ID NO: 3196 - DNA, 24 bp, Homo sapiens
ttttgaaagaggcaagagaagtgg

SEQ ID NO: 3197 - DNA, 24 bp, Homo sapiens
tttatgaagcacacacgataccag

SEQ ID NO: 3198 - DNA, 26 bp, Homo sapiens
aggatgctctgtctcttgctgtccag

SEQ ID NO: 3199 - DNA, 26 bp, Homo sapiens
gttggctgactgtgaatggtggtaaa

SEQ ID NO: 3200 - DNA, 26 bp, Homo sapiens
accagggcacttgcgggccagctgga

SEQ ID NO: 3201 - DNA, 26 bp, Homo sapiens
ttttcagttataagaagaatactagt

SEQ ID NO: 3202 - DNA, 23 bp, Homo sapiens
ggctatgctgctcaacaagacac

SEQ ID NO: 3203 - DNA, 22 bp, Homo sapiens
acatcctgctgattggtccatt

SEQ ID NO: 3204 - DNA, 21 bp, Homo sapiens
atgtagccatcattcttatgc

SEQ ID NO: 3205 - DNA, 23 bp, Homo sapiens
tgatggcttcattctgtgtaagg

SEQ ID NO: 3206 - DNA, 26 bp, Homo sapiens
tgaagagatccgacatcaagtgccca

SEQ ID NO: 3207 - DNA, 28 bp, Homo sapiens
ggagttacacgatgcctgcttacacctc

SEQ ID NO: 3208 - DNA, 21 bp, Homo sapiens
cacaagctgacacagcaagca

SEQ ID NO: 3209 - DNA, 23 bp, Homo sapiens
gccgccctgctgctgatgctacc

SEQ ID NO: 3210 - DNA, 28 bp, Homo sapiens
ctggtcacagagagacaaatcctgggtg

SEQ ID NO: 3211 - DNA, 24 bp, Homo sapiens
catcttttgaaccacggatagctg

SEQ ID NO: 3212 - DNA, 26 bp, Homo sapiens
cctagtttggggaatttttgagactc

SEQ ID NO: 3213 - DNA, 28 bp, Homo sapiens
gtgaaaacctatctcggctgggcacggg

SEQ ID NO: 3214 - DNA, 28 bp, Homo sapiens
gaccagcagacaaatcccagttcaatgc

SEQ ID NO: 3215 - DNA, 24 bp, Homo sapiens
ctgcggttgggtggcacagggctg

SEQ ID NO: 3216 - DNA, 26 bp, Homo sapiens
gaaatctcattctgcaacagccccac

SEQ ID NO: 3217 - DNA, 24 bp, Homo sapiens
acagagtgagattctgtttcaaaa

SEQ ID NO: 3218 - DNA, 24 bp, Homo sapiens
caacgggaaagccacagactgggg

SEQ ID NO: 3219 - DNA, 22 bp, Homo sapiens
agctaaactccgtctcaaaaaa

SEQ ID NO: 3220 - DNA, 23 bp, Homo sapiens
ttgggaggctgaggcaggtggat

SEQ ID NO: 3221 - DNA, 25 bp, Homo sapiens
aggagttcaagaccagcctggagca

SEQ ID NO: 3222 - DNA, 27 bp, Homo sapiens
gtgaacaaacagacctccagatgtggc

SEQ ID NO: 3223 - DNA, 26 bp, Homo sapiens
gcggctcacgcctgtaatcccagcac

SEQ ID NO: 3224 - DNA, 24 bp, Homo sapiens
ttataatgattttataaatgtact

SEQ ID NO: 3225 - DNA, 25 bp, Homo sapiens
ctcagccaagccacagacatggctg

SEQ ID NO: 3226 - DNA, 26 bp, Homo sapiens
tttgaatttttttttttttttttttt

SEQ ID NO: 3227 - DNA, 24 bp, Homo sapiens
aaatcagcatgttcttaggaggga

SEQ ID NO: 3228 - DNA, 22 bp, Homo sapiens
taggatgtattcataggggatg

SEQ ID NO: 3229 - DNA, 21 bp, Homo sapiens
ctcgtggctctcactgcaacg

SEQ ID NO: 3230 - DNA, 28 bp, Homo sapiens
ttttgaaatgtgagaaggttgggctggg

SEQ ID NO: 3231 - DNA, 28 bp, Homo sapiens
accacccagaacaagccagcacatttgc

SEQ ID NO: 3232 - DNA, 28 bp, Homo sapiens
gttattccgagaccattgtgtgtgagtc

SEQ ID NO: 3233 - DNA, 25 bp, Homo sapiens
tgaatccaacaaacgggaactggat

SEQ ID NO: 3234 - DNA, 21 bp, Homo sapiens
ctgggttcaagcaattctcct

SEQ ID NO: 3235 - DNA, 28 bp, Homo sapiens
atcatagttctcatagaccagggtttgc

SEQ ID NO: 3236 - DNA, 28 bp, Homo sapiens
catacccttttctcagcacagcagtgga

SEQ ID NO: 3237 - DNA, 26 bp, Homo sapiens
cctcattaccaccattccttgttaat

SEQ ID NO: 3238 - DNA, 23 bp, Homo sapiens
tctgccaggctctgctgcgaggc

SEQ ID NO: 3239 - DNA, 25 bp, Homo sapiens
attaagaaacctaataaagctccac

SEQ ID NO: 3240 - DNA, 24 bp, Homo sapiens
gttccgcctacccaggtccacacc

SEQ ID NO: 3241 - DNA, 28 bp, Homo sapiens
ttaggctggtcttgaactcctgggctca

SEQ ID NO: 3242 - DNA, 28 bp, Homo sapiens
gcccaacctcatccccacacagacctcc

SEQ ID NO: 3243 - DNA, 25 bp, Homo sapiens
tgctatgttgcccaggcttgtcttc

SEQ ID NO: 3244 - DNA, 23 bp, Homo sapiens
gcctccaggaaggtgtgggtggc

SEQ ID NO: 3245 - DNA, 28 bp, Homo sapiens
gtggcaggcttcagggtggtaggacagg

SEQ ID NO: 3246 - DNA, 24 bp, Homo sapiens
atgaacacccacaacgcctgaact

SEQ ID NO: 3247 - DNA, 21 bp, Homo sapiens
gtggctcacacctgtaatccc

SEQ ID NO: 3248 - DNA, 22 bp, Homo sapiens
acacctataatcccagcacttt

SEQ ID NO: 3249 - DNA, 27 bp, Homo sapiens
tcccctgcctcatcttcctcacctggc

SEQ ID NO: 3250 - DNA, 28 bp, Homo sapiens
ggacctttagacatcggtggagaacagg

SEQ ID NO: 3251 - DNA, 22 bp, Homo sapiens
ggccagctggagttccaggtga

SEQ ID NO: 3252 - DNA, 23 bp, Homo sapiens
aatctctttgactacaccattct

SEQ ID NO: 3253 - DNA, 22 bp, Homo sapiens
caaacccttcattttttatcct

SEQ ID NO: 3254 - DNA, 21 bp, Homo sapiens
tcattgaaatctaaaattcag

SEQ ID NO: 3255 - DNA, 23 bp, Homo sapiens
tcctttagagcaacaaaaatgga

SEQ ID NO: 3256 - DNA, 26 bp, Homo sapiens
ctttcctagtctatcatgtgtctttt

SEQ ID NO: 3257 - DNA, 26 bp, Homo sapiens
gcccctcacttccgccacactccgtg

SEQ ID NO: 3258 - DNA, 21 bp, Homo sapiens
tgggaggccaaggtgggagga

SEQ ID NO: 3259 - DNA, 28 bp, Homo sapiens
caacagcctgactcaatgaccagtaaag

SEQ ID NO: 3260 - DNA, 27 bp, Homo sapiens
gctgggcggatggatggcttgagtctg

SEQ ID NO: 3261 - DNA, 24 bp, Homo sapiens
ctcgggtgcagagaggagggtttg

SEQ ID NO: 3262 - DNA, 26 bp, Homo sapiens
taattggtactttcatgggccaagaa

SEQ ID NO: 3263 - DNA, 28 bp, Homo sapiens
gctttccacccctacaatcccaagtgtt

SEQ ID NO: 3264 - DNA, 28 bp, Homo sapiens
gagtcacaaagcaggagggagaacaggt

SEQ ID NO: 3265 - DNA, 28 bp, Homo sapiens
gggtggtgaggaaacactaaggagaagg

SEQ ID NO: 3266 - DNA, 26 bp, Homo sapiens
cccggccgactcttttttttcttaat

SEQ ID NO: 3267 - DNA, 24 bp, Homo sapiens
attgaaatatctgcctttatctta

SEQ ID NO: 3268 - DNA, 24 bp, Homo sapiens
caggcaacatcccaggggctgaaa

SEQ ID NO: 3269 - DNA, 22 bp, Homo sapiens
aaaacaacaaaacccataaaat

SEQ ID NO: 3270 - DNA, 28 bp, Homo sapiens
gggagtgagaaagaggaagtatcaaata

SEQ ID NO: 3271 - DNA, 25 bp, Homo sapiens
ctcccgccccgccccgccccggaca

SEQ ID NO: 3272 - DNA, 24 bp, Homo sapiens
cgcataatcaaagccatcagtgac

SEQ ID NO: 3273 - DNA, 24 bp, Homo sapiens
ccccttccaacccaatcaccccac

SEQ ID NO: 3274 - DNA, 24 bp, Homo sapiens
agagctaacattttattctgtgtt

SEQ ID NO: 3275 - DNA, 25 bp, Homo sapiens
gggggcatggacccggccttgtttg

SEQ ID NO: 3276 - DNA, 26 bp, Homo sapiens
aacaatagaaatgacccctcagggta

SEQ ID NO: 3277 - DNA, 24 bp, Homo sapiens
gcctttatgagaaaccaggcatta

SEQ ID NO: 3278 - DNA, 22 bp, Homo sapiens
ggggaagggactctcctggggt

SEQ ID NO: 3279 - DNA, 21 bp, Homo sapiens
ctccgtgtactactgggcaat

SEQ ID NO: 3280 - DNA, 25 bp, Homo sapiens
agaacaagaagtctgaggagagtag

SEQ ID NO: 3281 - DNA, 24 bp, Homo sapiens
cccacccacctccacctgcccacg

SEQ ID NO: 3282 - DNA, 28 bp, Homo sapiens
taagggcattctcctaataactgtgatg

SEQ ID NO: 3283 - DNA, 25 bp, Homo sapiens
cacaagacttctggcttctcaagcc

SEQ ID NO: 3284 - DNA, 21 bp, Homo sapiens
ggaatctgtgacatagcatat

SEQ ID NO: 3285 - DNA, 28 bp, Homo sapiens
tgaagttgtgccaaagccacctgtgcca

SEQ ID NO: 3286 - DNA, 28 bp, Homo sapiens
taaaagacctgatgtatttcctaccagc

SEQ ID NO: 3287 - DNA, 26 bp, Homo sapiens
acttaaagaacagaatagttaagtaa

SEQ ID NO: 3288 - DNA, 26 bp, Homo sapiens
gctcttgtggagaactggagacctta

SEQ ID NO: 3289 - DNA, 25 bp, Homo sapiens
cgggtgcagagaggagggtttggtg

SEQ ID NO: 3290 - DNA, 28 bp, Homo sapiens
ggacctgagtgcgaggagttgtgtgggc

SEQ ID NO: 3291 - DNA, 26 bp, Homo sapiens
ctgtctggctggtgctggtgtctctg

SEQ ID NO: 3292 - DNA, 23 bp, Homo sapiens
gcgggagccagggcagagagact

SEQ ID NO: 3293 - DNA, 25 bp, Homo sapiens
ctagaaaccattaatctactatctg

SEQ ID NO: 3294 - DNA, 28 bp, Homo sapiens
agccacggcagcccctgtaatcaacccg

SEQ ID NO: 3295 - DNA, 23 bp, Homo sapiens
gctgcctgctgttcccgtgccag

SEQ ID NO: 3296 - DNA, 24 bp, Homo sapiens
accccaaacacggaggccccaagg

SEQ ID NO: 3297 - DNA, 21 bp, Homo sapiens
ccatatactcatttttaagct

SEQ ID NO: 3298 - DNA, 22 bp, Homo sapiens
tgataatataatgttatattaa

SEQ ID NO: 3299 - DNA, 28 bp, Homo sapiens
ccttgtttcaccccacacactgacggcg

SEQ ID NO: 3300 - DNA, 28 bp, Homo sapiens
gttggctgactgtgaatggtggtaaaat

SEQ ID NO: 3301 - DNA, 22 bp, Homo sapiens
tactagtaaaaacattacactt

SEQ ID NO: 3302 - DNA, 40 bp, Homo sapiens
ttgcttgagttcaggagttcgaacaagatattaagtatta

SEQ ID NO: 3303 - DNA, 40 bp, Homo sapiens
gtagctagatacagagtgtcgattgtcatgttttcactga

SEQ ID NO: 3304 - DNA, 40 bp, Homo sapiens
tcacctgaggtttggagttcgaatgtttctctaatgcatg

SEQ ID NO: 3305 - DNA, 40 bp, Homo sapiens
aggtgctatgcttatgactcgagacatgtggagtcacccc

SEQ ID NO: 3306 - DNA, 40 bp, Homo sapiens
gttagctcagagcctcagtcgaaggaagcttaggaatata

SEQ ID NO: 3307 - DNA, 33 bp, Homo sapiens
aggtcagaaattcgacctccttgctcaactgtc

SEQ ID NO: 3308 - DNA, 40 bp, Homo sapiens
gtgtatgtcaacaatttctcgaactcccaggttcaaacga

SEQ ID NO: 3309 - DNA, 32 bp, Homo sapiens
agctggtcggtattcccctcgctctcgagtaa

SEQ ID NO: 3310 - DNA, 36 bp, Homo sapiens
agggagtagaggaatcgggagccagtgttcgaataa

SEQ ID NO: 3311 - DNA, 40 bp, Homo sapiens
tttagagctgttttctcctcgaagggctggttgtactgcg

SEQ ID NO: 3312 - DNA, 40 bp, Homo sapiens
gatcactaggtcaggagttcgaaacccacactgcaagtat

SEQ ID NO: 3313 - DNA, 36 bp, Homo sapiens
aggcaggcggatcacgaggtcaatagatcgatagga

SEQ ID NO: 3314 - DNA, 36 bp, Homo sapiens
agttcactgcagcctcgatacacattttcaaagtgc

SEQ ID NO: 3315 - DNA, 33 bp, Homo sapiens
tgagcccagaagttcgagttttccccttgctcc

SEQ ID NO: 3316 - DNA, 40 bp, Homo sapiens
tcacttgaggtaaggagttcgaaaattagcagaaaaaagc

SEQ ID NO: 3317 - DNA, 40 bp, Homo sapiens
tcacctgagttcaggagttcgagatgacagctaaacataa

SEQ ID NO: 3318 - DNA, 40 bp, Homo sapiens
gttagctcagagcctcagtcgagtcatatttgaatgtcta

SEQ ID NO: 3319 - DNA, 40 bp, Homo sapiens
atcacttgaggttagggttcgaccagcagcaaagacagca

SEQ ID NO: 3320 - DNA, 38 bp, Homo sapiens
tgcagtgagccgagatcatgccattatactcgaaacag

SEQ ID NO: 3321 - DNA, 40 bp, Homo sapiens
tcacttgagcctaggaagtcgatgtttcttgaattgtttt

SEQ ID NO: 3322 - DNA, 32 bp, Homo sapiens
atcacttgaacccgggagtcgagatcagcctg

SEQ ID NO: 3323 - DNA, 35 bp, Homo sapiens
tgaggtcagaaattcgaaacgaagggctaagcccc

SEQ ID NO: 3324 - DNA, 40 bp, Homo sapiens
tcacttgaggccagggattcgatcctcaagctaagaataa

SEQ ID NO: 3325 - DNA, 40 bp, Homo sapiens
tgcgtcctgtcactctcctcgaggttatatttattctaat

SEQ ID NO: 3326 - DNA, 40 bp, Homo sapiens
ttctcctgcctcagactctcgatacataaggacttaggaa

SEQ ID NO: 3327 - DNA, 40 bp, Homo sapiens
gataagcttaagaagagttcgagacacttattttggagat

SEQ ID NO: 3328 - DNA, 40 bp, Homo sapiens
tcacctgaggtcaggaattcgaaaaaaaaaaaaaaaagaa

SEQ ID NO: 3329 - DNA, 40 bp, Homo sapiens
gttagctcagagcctcagtcgaatacacactcattgaagg

SEQ ID NO: 3330 - DNA, 32 bp, Homo sapiens
tgggcagatcacgaggacaggagatcgagcct

SEQ ID NO: 3331 - DNA, 33 bp, Homo sapiens
tgtacatcgaggtgagagtgagctgagatggcg

SEQ ID NO: 3332 - DNA, 35 bp, Homo sapiens
aggcagatcatctgaggttgggagttcgaagacac

SEQ ID NO: 3333 - DNA, 40 bp, Homo sapiens
cataagactaaggttctgtcgatacatatttgtttaatga

SEQ ID NO: 3334 - DNA, 40 bp, Homo sapiens
atgttggtcagtctggtctcgaagtcactcctaactcata

SEQ ID NO: 3335 - DNA, 35 bp, Homo sapiens
agcttgtctaggtaaagttccgggtcgaataggcc

SEQ ID NO: 3336 - DNA, 38 bp, Homo sapiens
aaggcaggtggatcacgaggtcagcagatcgatacata

SEQ ID NO: 3337 - DNA, 40 bp, Homo sapiens
tcacctgaggccaggaattcgaaatactatgctaagattc

SEQ ID NO: 3338 - DNA, 35 bp, Homo sapiens
taggcctcgaaaggtcattataaggatggagtgag

SEQ ID NO: 3339 - DNA, 40 bp, Homo sapiens
aaacattaacttggtgtatcgaagggctggttgtactgcg

SEQ ID NO: 3340 - DNA, 37 bp, Homo sapiens
tgcaggtgtgtggttaacactatccttcgacctgtac

SEQ ID NO: 3341 - DNA, 36 bp, Homo sapiens
agtggtgccatcatagctcactgcagcctcgaataa

SEQ ID NO: 3342 - DNA, 39 bp, Homo sapiens
tggccaagtgcaccacgtgaacaaaacaatctcgattgg

SEQ ID NO: 3343 - DNA, 40 bp, Homo sapiens
atcacttgagctcaggattcgaaaacattaactgaaagaa

SEQ ID NO: 3344 - DNA, 39 bp, Homo sapiens
acggaatgacatcgagaaagtggaactggtaagcatagg

SEQ ID NO: 3345 - DNA, 31 bp, Homo sapiens
aggtgagttcgagctgccgtgtgtcccatag

SEQ ID NO: 3346 - DNA, 40 bp, Homo sapiens
aggtgctatgcttatgactcgagcttgcactccggtaccc

SEQ ID NO: 3347 - DNA, 40 bp, Homo sapiens
caagagaattgcttgaactcgattcagcatatgaagggga

SEQ ID NO: 3348 - DNA, 40 bp, Homo sapiens
ttgcttgagcctgaaaggtcgaaaaataaaaatgaaaata

SEQ ID NO: 3349 - DNA, 37 bp, Homo sapiens
aggcaggaggatcgcttgaacccaggagttcgaaata

SEQ ID NO: 3350 - DNA, 32 bp, Homo sapiens
agccgcaagaagtacagcagcacctcgatctc

SEQ ID NO: 3351 - DNA, 40 bp, Homo sapiens
agcctaccggtgctgcactcgacagaaagcttaagggcaa

SEQ ID NO: 3352 - DNA, 40 bp, Homo sapiens
taatacttaatatcttgttcgaactcctgaactcaagcaa

SEQ ID NO: 3353 - DNA, 40 bp, Homo sapiens
tcagtgaaaacatgacaatcgacactctgtatctagctac

SEQ ID NO: 3354 - DNA, 40 bp, Homo sapiens
catgcattagagaaacattcgaactccaaacctcaggtga

SEQ ID NO: 3355 - DNA, 40 bp, Homo sapiens
ggggtgactccacatgtctcgagtcataagcatagcacct

SEQ ID NO: 3356 - DNA, 40 bp, Homo sapiens
tatattcctaagcttccttcgactgaggctctgagctaac

SEQ ID NO: 3357 - DNA, 33 bp, Homo sapiens
agacagttgagcaaggaggtcgaatttctgacc

SEQ ID NO: 3358 - DNA, 40 bp, Homo sapiens
tcgtttgaacctgggagttcgagaaattgttgacatacac

SEQ ID NO: 3359 - DNA, 34 bp, Homo sapiens
accatcttctgccacttttactcgagagcgaggg

SEQ ID NO: 3360 - DNA, 35 bp, Homo sapiens
ttattcgaacactggctcccgattcctctactccc

SEQ ID NO: 3361 - DNA, 40 bp, Homo sapiens
cgcagtacaaccagcccttcgaggagaaaacagctctaaa

SEQ ID NO: 3362 - DNA, 40 bp, Homo sapiens
atacttgcagtgtgggtttcgaactcctgacctagtgatc

SEQ ID NO: 3363 - DNA, 34 bp, Homo sapiens
tcctatcgatctattgacctcgtgatccgcctgc

SEQ ID NO: 3364 - DNA, 36 bp, Homo sapiens
agcactttgaaaatgtgtatcgaggctgcagtgaac

SEQ ID NO: 3365 - DNA, 36 bp, Homo sapiens
aaactcgaacttctgggctcaagcaattctcctgcc

SEQ ID NO: 3366 - DNA, 40 bp, Homo sapiens
gcttttttctgctaattttcgaactccttacctcaagtga

SEQ ID NO: 3367 - DNA, 40 bp, Homo sapiens
ttatgtttagctgtcatctcgaactcctgaactcaggtga

SEQ ID NO: 3368 - DNA, 40 bp, Homo sapiens
tagacattcaaatatgactcgactgaggctctgagctaac

SEQ ID NO: 3369 - DNA, 40 bp, Homo sapiens
tgctgtctttgctgctggtcgaaccctaacctcaagtgat

SEQ ID NO: 3370 - DNA, 38 bp, Homo sapiens
tcctgtttcgagtataatggcatgatctcggctcactg

SEQ ID NO: 3371 - DNA, 40 bp, Homo sapiens
aaaacaattcaagaaacatcgacttcctaggctcaagtga

SEQ ID NO: 3372 - DNA, 32 bp, Homo sapiens
tcaggctgatctcgactcccgggttcaagtga

SEQ ID NO: 3373 - DNA, 38 bp, Homo sapiens
agcccttcgtttcgaatttctgacctcaagtgatctgc

SEQ ID NO: 3374 - DNA, 40 bp, Homo sapiens
ttattcttagcttgaggatcgaatccctggcctcaagtga

SEQ ID NO: 3375 - DNA, 40 bp, Homo sapiens
attagaataaatataacctcgaggagagtgacaggacgca

SEQ ID NO: 3376 - DNA, 40 bp, Homo sapiens
ttcctaagtccttatgtatcgagagtctgaggcaggagaa

SEQ ID NO: 3377 - DNA, 40 bp, Homo sapiens
atctccaaaataagtgtctcgaactcttcttaagcttatc

SEQ ID NO: 3378 - DNA, 40 bp, Homo sapiens
ttcttttttttttttttttcgaattcctgacctcaggtga

SEQ ID NO: 3379 - DNA, 40 bp, Homo sapiens
ccttcaatgagtgtgtattcgactgaggctctgagctaac

SEQ ID NO: 3380 - DNA, 32 bp, Homo sapiens
tacaggctcgatctcctgtcctcgtgatctgc

SEQ ID NO: 3381 - DNA, 34 bp, Homo sapiens
tctcagctcactctcacctcgatgtacaagacag

SEQ ID NO: 3382 - DNA, 35 bp, Homo sapiens
tgtcttcgaactcccaacctcagatgatctgcctg

SEQ ID NO: 3383 - DNA, 40 bp, Homo sapiens
tcattaaacaaatatgtatcgacagaaccttagtcttatg

SEQ ID NO: 3384 - DNA, 40 bp, Homo sapiens
tatgagttaggagtgacttcgagaccagactgaccaacat

SEQ ID NO: 3385 - DNA, 34 bp, Homo sapiens
aggcctattcgacccggaactttacctagacaag

SEQ ID NO: 3386 - DNA, 33 bp, Homo sapiens
tgtatcgatctgctgacctcgtgatccacctgc

SEQ ID NO: 3387 - DNA, 40 bp, Homo sapiens
gaatcttagcatagtatttcgaattcctggcctcaggtga

SEQ ID NO: 3388 - DNA, 36 bp, Homo sapiens
actccatccttataatgacctttcgaggcctacccc

SEQ ID NO: 3389 - DNA, 40 bp, Homo sapiens
cgcagtacaaccagcccttcgatacaccaagttaatgttt

SEQ ID NO: 3390 - DNA, 37 bp, Homo sapiens
agtacaggtcgaaggatagtgttaaccacacacctgc

SEQ ID NO: 3391 - DNA, 35 bp, Homo sapiens
ttattcgaggctgcagtgagctatgatggcaccac

SEQ ID NO: 3392 - DNA, 38 bp, Homo sapiens
tcccaatcgagattgttttgttcacgtggtgcacttgg

SEQ ID NO: 3393 - DNA, 40 bp, Homo sapiens
ttctttcagttaatgttttcgaatcctgagctcaagtgat

SEQ ID NO: 3394 - DNA, 39 bp, Homo sapiens
acccctatgcttaccagttccactttctcgatgtcattc

SEQ ID NO: 3395 - DNA, 31 bp, Homo sapiens
tatgggacacacggcagctcgaactcacctg

SEQ ID NO: 3396 - DNA, 40 bp, Homo sapiens
gggtaccggagtgcaagctcgagtcataagcatagcacct

SEQ ID NO: 3397 - DNA, 40 bp, Homo sapiens
tccccttcatatgctgaatcgagttcaagcaattctcttg

SEQ ID NO: 3398 - DNA, 40 bp, Homo sapiens
tattttcatttttatttttcgacctttcaggctcaagcaa

SEQ ID NO: 3399 - DNA, 35 bp, Homo sapiens
tatttcgaactcctgggttcaagcgatcctcctgc

SEQ ID NO: 3400 - DNA, 32 bp, Homo sapiens
tgagatcgaggtgctgctgtacttcttgcggc

SEQ ID NO: 3401 - DNA, 40 bp, Homo sapiens
ttgcccttaagctttctgtcgagtgcagcaccggtaggct